(19) United States
(12) Patent Application Publication
(10) Pub. No.: US 2004/0009569 A1
(43) Pub. Date: Jan. 15, 2004

(54) Kinase crystal structures and materials and methods for kinase activation

(75) Inventors: David Barford, London (GB); Jing Yang, Middlesex (GB); Brian Arthur Hemmings, Bettingen (CH); Peter David Cron, Basel (CH)

Correspondence Address: GRAY CARY WARE FREDENRICH, 1625 MASSACHUSETTS AVENUE, NW, SUITE 300, WASHINGTON, DC, 20036-2247, US

(73) Assignee: The Cancer Research Institute

(21) Appl. No.: 10/217,555

(22) Filed: Aug. 14, 2002

(30) Foreign Application Priority Data
Aug. 14, 2001 (GB) 0119860.5
May 1, 2002 (GB) 0209985.1

(51) Int. Cl. G06F 19/00; G01N 33/48; G01N 33/50; C12N 9/12

(52) U.S. Cl. 435/194; 702/19

(57) ABSTRACT

Disclosed are crystallisable mutants of protein kinase B/Akt, crystals of these mutants, and X-ray coordinate data for the crystals. Methods of use of the coordinate data for identification of modulators of protein kinase activity and for structural analysis of other protein kinases are provided. Also provided are methods of activating protein kinases, in particular AGC kinases, using peptide or non-peptide mimetics of sequences from protein kinase B/Akt, or other AGC protein kinases such as PRK2.

### FIELD OF THE INVENTION

The present invention relates to the enzyme protein kinase B (PKB/Akt), and in particular its crystal structure and the use of this structure in drug discovery.

### BACKGROUND TO THE INVENTION

Protein kinase B (PKB/Akt) is a component of an intracellular signalling pathway of fundamental importance that functions to exert the effects of growth and survival factors, and which mediates the response to insulin and inflammatory signals (Datta et al., 1999; Brazil and Hemmings, 2001). The enzyme is rapidly activated by phosphorylation following stimulation of phosphoinositide 3-kinase, and generation of the lipid second messenger phosphatidylinositol 3,4,5 trisphosphate [PtdIns(3,4,5)P₃]. Activation of PKB occurs by a multi-step mechanism. PKB is first recruited to the membrane by association with PtdIns(3,4,5)P₃ mediated by its N-terminal pleckstrin homology domain in a process that also induces a conformational change of the protein. In this state, PKB is a substrate for phosphorylation at two regulatory sites by membrane-localised kinases (Meier et al. 1997). PDK1 phosphorylates PKB on a Thr residue (Thr-308 of PKBα, Thr-309 of PKBβ, Thr-305 of PKBγ) within the activation segment, stimulating its activity by 30-fold (Alessi et al., 1996; 1997). A distinct kinase activity, termed PDK2, phosphorylates PKB at a Ser residue of a C-terminal hydrophobic motif (Ser 473 of PKBα, Ser-474 of PKBβ, Ser-472 of PKBγ). Phosphorylation of Ser-474 promotes a 7-10-fold stimulation (Alessi et al., 1996), which is synergistic with pThr-309 so that phosphorylation of both sites results in an ˜300-fold elevation of protein kinase activity. Whereas PDK1 is well characterised, the identity of PDK2 (also designated Ser-473 Kinase) remains controversial.

Activated PKB phosphorylates numerous cytosolic and nuclear proteins to regulate cell metabolism, growth and survival. In the insulin signalling pathway, PKB phosphorylates GSK-3, PFK2 and mTOR, inducing glycogenesis and protein synthesis, and regulates glucose uptake by promoting the translocation of Glut4 to the plasma membrane. Cell survival and transformation are controlled by phosphorylation of BAD, caspase-9, forkhead transcription factors and IKB kinase, promoting proliferation and suppressing cell apoptosis (Datta et al., 1999). A mechanism by which PKB stimulates cell cycle progression is by phosphorylation of the CDK inhibitors p21^{WAFl} and p27^{KiPl}, causing their retention in the cytoplasm (Zhou et al., 2001), whereas in contrast, PKB mediates nuclear localisation of mdm2 and subsequent regulation of the mdm2/p53 pathway (Mayo and Donner, 2001). In humans, the three isoforms of PKB are highly conserved, with a mean sequence identity of 73%, and share the same regulatory phosphorylation sites. However, a splice variant of PKBγ lacks the C-terminal regulatory phosphorylation site, and interestingly, the specific activity of this splice variant, isolated from stimulated cells, is ˜10-fold lower than the full length γ isoform, a value which is consistent with the role of the C-terminal phosphorylation site to stimulate PKB activity (Brodbeck et al., 2001). CTMP is a negative regulator of PKBα, which by binding to the C-terminal region of the protein, suppresses phosphorylation of Thr-308 and Ser-473 (Maira et al., 2001).

PKB plays an important role in the generation of human malignancy. The enzyme is the cellular homologue of v-Akt, an oncogene of the transforming murine leukaemia virus AKT8 isolated from a mouse lymphoma (Staal et al., 1977). Viral-Akt is a fusion of the viral Gag protein with the PKBα sequence (Bellacosa et al., 1991). Myristoylation of the Gag sequence targets v-Akt to the cell membrane, resulting in its constitutive phosphorylation. The genes for the α and β isoforms of PKB are over-expressed and amplified in ovarian, prostate, pancreatic, gastric, and breast tumours (Testa and Bellacosa, 2001). Compelling evidence linking PKB to oncogenesis stems from the elucidation of the mechanism of the PTEN tumour suppressor gene. PTEN is one of the most commonly mutated genes in human cancer and somatic deletions or mutations of PTEN have been identified in glioblastomas, melanoma and prostate cancers, and are associated with increased susceptibility to breast and thyroid tumours (Cantley and Neel, 1999). PTEN negatively regulates the PI-3 kinase/PKB pathway by dephosphorylating PtdIns(3,4,5)P₃ on the D-3 position, and therefore loss of PTEN activity leads to a constitutive cell survival stimulus (Maehama and Dixon., 1998; Myers et al., 1998).

Protein kinase B is a member of the AGC-family of serine/threonine specific protein kinases that also includes PKA, PKC, PDK1 and the p70 and p90 S6-kinases (Coffer and Woodgett, 1991; Jones et al., 1991a). As well as being structurally related, AGC-protein kinases share numerous functional similarities such as activation in response to second messengers and dependence on phosphorylation for activity. Members of the family are phosphorylated on a conserved Thr-residue within their activation segment. In vitro PDK1 is capable of phosphorylating AGC-kinases on this position (Vanhaesebroeck and Alessi, 2000), although recent studies using PDK1 deficient ES cells suggest that PDK1 activity is only necessary for PKB and a subset of other AGC-kinases (Williams et al., 2000). The site of C-terminal regulatory phosphorylation of PKB (Ser-474) is within a hydrophobic activation sequence motif (F-x-x-F-[S/T]-Y), conserved within a large proportion of AGC-kinases (Keranen et al., 1995; Pearson et al., 1995). In PKB, substitution of Ser-474 with Asp mimics Ser-474 phosphorylation (Alessi et al., 1996), and significantly, some atypical PKC isoforms and PRK2 (PKC related kinase-2) have Asp or Glu residues at this position. PKA requires phosphorylation of the activation segment Thr residue (Thr-197) for activity (Yonemoto et al., 1997), although this is a constitutive site of phosphorylation, and unlike other AGC-kinases, is resistant to dephosphorylation by protein phosphatases (Shoji et al., 1979). The hydrophobic motif of PKA is also unusual and comprises the sequence -Phe-Thr-Glu-Phe-350, with Phe-350 corresponding to the C-terminus of the PKA catalytic subunit, and therefore the enzyme lacks a site of regulatory phosphorylation. In the structure of PKA, the motif lies within a surface groove formed in the N-terminal lobe, with the side-chains of the two Phe-residues buried deep into the groove (Knighton et al., 1991a,b; Bossemeyer et al., 1993). Other AGC-kinases are likely to have an equivalent groove, and for PDK1, the groove is thought to allow recognition of specific target kinase substrates via their phosphorylated regulatory segment sequences, although this interaction has been suggested not to be essential for phosphorylation of PKB by PDK1 (Biondi et al., 2000; 2001).

In order to understand the mechanism of activation of PKB by phosphorylation, and as a framework for the rational development of modulators of PKB activity, knowledge of a PKB protein structure would be extremely valuable. Such knowledge would significantly assist the rational design of novel therapeutics for, e.g. the treatment of diabetes, cancer, neurodegeneration and erectile dysfunction, based on PKB modulators.

### DEFINITIONS

In the following by “binding site” we mean a site (such as an atom, a functional group of an amino acid residue or a plurality of such atoms and/or groups) in a PKB binding cavity which may bind to an agent compound such as a candidate modulator (e.g. inhibitor). Depending on the particular molecule in the cavity, sites may exhibit attractive or repulsive binding interactions, brought about by charge, steric considerations and the like.

By “AGC kinase” is meant any protein kinase comprising a sequence which has a sequence identity of equal to or greater than 35% at the amino acid level with residues 37-350 of the catalytic subunit of PKA (Shoji et al., 1983). Determination of percentage sequence identity may be performed with the AMPS package as described by Barton (1994). AGC kinases are also described in detail by Hanks and Hunter, FASEB J. (1995) 9: 576, and Hardie, G. and Hanks, S. (eds) The Protein Kinase Facts Book—Protein Serine Kinases (1995) Academic Press Ltd., London).

By “fitting”, is meant determining by manual, automatic, or semi-automatic means, interactions between one or more atoms of an agent molecule and one or more atoms or binding sites of the PKB, and calculating the extent to which such interactions are stable. Various computer-based methods for fitting are described further herein.

By “root mean square deviation” we mean the square root of the arithmetic mean of the squares of the deviations from the mean.

By a “computer system” we mean the hardware means, software means and data storage means used to analyse atomic coordinate data. The minimum hardware means of the computer-based systems of the present invention comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” we mean any media which can be read and accessed directly by a computer e.g. so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

### DISCLOSURE OF THE INVENTION

The present invention is at least partly based on overcoming several technical hurdles: the present inventors have (i) produced PKBβ crystals of suitable quality for performing X-ray diffraction analyses, (ii) collected X-ray diffraction data from the crystals, (iii) determined the three-dimensional structure of PKBβ, and (iv) identified binding sites on the enzyme which are likely to be involved in the enzymatic reaction.

In order to understand the nature of the mechanism of regulation of PKB by PIP₃, the N-terminal PH domain and phosphorylation of the regulatory Thr and Ser phosphorylation sites, the present inventors have determined the crystal structures of PKBβ and have generated Sf9/baculovirus expression systems for full length PKBα, PKBβ, PKBγ and PDK1 and also a number of modified forms of the three PKB isoforms.

Limited trypsinolysis of full length PKBβ purified from Sf9 cells led to the identification of a protease resistant domain with an N-terminus at Lys-146, which we refer to as ΔPH-PKB. Lys-146 is located within the structurally diverse region linking the pleckstrin homology (PH) and kinase domains of PKB, close to the N-terminus of the corresponding β1-strand of PKA. During the course of the purification, partial cleavage of a C-terminal 3 kDa fragment was observed, suggesting conformational flexibility at the C-terminus of the protein. Human PKBα, PKBβ and PKBγ sequences are structurally diverse within a 12 residue region C-terminal to the conserved PP(D/E) motif (residues 452-454 of PKBβ), preceding the C-terminal hydrophobic motif, and corresponding to the C-terminus of the PKBγ splice variant. Using this information, the present inventors constructed a number of new PKB baculovirus Fastbac entry vectors for the generation of PKB insect cell/baculovirus expression systems, and expressed the α and β-isoforms of PKB as the kinase domain, with an N-terminus at Lys-146 (i.e. lacking the PH domain), with and without the C-terminal 21 residues that includes the hydrophobic regulatory segment. These two kinase domains are termed ΔPH-PKB and ΔPH-PKB-ΔC, respectively.

Thus, using this limited proteolysis, the inventors have defined a stable, compact, crystallisable domain of PKB. The systems used express high levels of protein, that the inventors have purified to homogeneity. Moreover, the inventors have expressed PDK1 using the insect cell/baculovirus system and MAPKAPK2 in the E. coli expression system to enable phosphorylation of PKBβ on Thr309 and Ser474, respectively.

To prepare defined phosphorylated states of PKB, phosphorylation and dephosphorylation reactions were performed using PDK1 (for pThr-309) and the non-specific λ-protein phosphatase, respectively. Distinct phosphorylated states of the protein were resolved using hydrophobic interaction chromatography.

The phosphorylation state of the protein was analysed by Western blots using phospho-specific antibodies, and the stoichiometry and sites of phosphorylation were quantitatively assessed by mass spectroscopic analysis of trypsin-generated peptides of the protein.

The inventors have succeeded in the expression, purification, crystallisation and structure determination of three forms of PKBβ, that differ in their state of phosphorylation.

The three crystal forms of human PKBβ are; (i) pΔPH-PKB-ΔC (residues 146 to 460, phosphorylated in vitro on Thr-309), (ii) ΔPH-PKB-ΔC(residues 146 to 460, not phosphorylated on Thr-309), and (iii) ΔPH-PKBβ (residues 146 to 481, dephosphorylated in vitro)

Two batches of crystals were prepared for crystal form (i), i.e. pΔPH-PKB-ΔC. Sets of coordinate data, having different resolutions, are provided for each of these crystals. thus, data are provided for four crystals in total.

All four crystals of PKB belong to the same space group and have similar cell dimensions. Summary data for the higher resolution pΔPH-PKB-ΔC crystal, as well as the ΔPH-PKB-ΔC and ΔPH-PKB crystals are shown in Table 1.

The two crystal preparations of pΔPH-PKB-ΔC diffract to 2.8 and 2.3 Å resolution, when exposed to synchrotron radiation, whereas ΔPH-PKB-ΔC and ΔPH-PKB diffract to 2.7 Å and 2.5 Å respectively.

The structure of PKB was solved by means of molecular replacement using the ternary complex of mouse PKA (Knighton et al., 1991) as a search object. During initial stages of the refinement, the relative orientations of the N- and C-terminal lobes of the kinase domain were refined, prior to atomic positional refinement (Table 1).

The resultant structures are believed to provide important insights into the structure activity relationships in the full length PKBβ and its highly homologous isoforms (see Brodbeck et al. 1999). For brevity, as used herein, unless the context demands otherwise, the term PKB is used to encompass full or part-length molecules of any of the three isoforms, which may not or may be phosphorylated e.g. ‘PKBβ’ encompasses the full length PKBβ molecule or a truncated form such as ΔPH-PKBβ (residues 146-481) or ΔPH-PKBβ-ΔC (residues 146-460).

In general aspects, the present invention is concerned with identifying or obtaining agent compounds for modulating PKB activity, and in preferred embodiments identifying or obtaining actual agent compounds which are inhibitors or activators. Where methods of identifying or modelling inhibitors are described hereinafter, the skilled person will appreciate that the processes may be applied analogously to other modulators such as activators.

Crystal structure information presented herein is useful in designing potential modulators and modelling them or their potential interaction with PKB binding cavities, for example, the PKB substrate binding cavity, ATP binding site, or other region of interest (e.g. the hydrophobic motif, or regulatory phosphorylation sites), preferably the ATP binding site. Potential modulators may be brought into contact with PKB to test for ability to interact with the PKB binding cavity. Actual modulators may be identified from among potential modulators synthesized following design and model work performed in silico. A modulator identified using the present invention may be formulated into a composition, for instance a composition comprising a pharmaceutically acceptable excipient, and may be used in the manufacture of a medicament for use in a method of treatment. These and other aspects and embodiments of the present invention are discussed below.

In a first aspect, the present invention provides a crystal of PKBβ having a tetragonal space group P4₁2₁2, and unit cell dimensions of a=149.33 Å, b=149.33 Å, c=39.77 Å, or more generally a=149.33±0.5 Å, b=149.33±0.5 Å, c=39.77±0.5 Å, more preferably a=149.33±0.2 Å, b=149.33±0.2 Å, c=39.77±0.2 Å.

The present invention further provides a crystal of PKBβ having a tetragonal space group P4₁2₁2, and unit cell dimensions of a=148.40 Å, b=148.40 Å, c=38.55 Å, or more generally a=148.40±0.5 Å, b=148.40±0.5 Å, c=38.55±0.5 Å, more preferably a=148.40±0.2 Å, b=148.40±0.2 Å, c=38.55±0.2 Å.

The present invention further provides a crystal of PKBβ having a tetragonal space group P4₁2₁2, and unit cell dimensions of a=149.70 Å, b=149.70 Å, c=39.19 Å, or more generally a=149.70±0.5 Å, b=149.70±0.5 Å, c=39.19±0.5 Å, more preferably a=149.70±0.2 Å, b=149.70±0.2 Å, c=39.19±0.2 Å.

The present invention further provides a crystal of PKBβ having a tetragonal space group P4₁2₁2, and unit cell dimensions of a=149.52 Å, b=149.52 Å, c=39.06 Å, or more generally a=149.52±0.5 Å, b=149.52±0.5 Å, c=39.06±0.5 Å, more preferably a=149.52±0.2 Å, b=149.52±0.2 Å, c=39.06±0.2 Å.

Alternatively, or additionally, the crystal may have the three dimensional atomic coordinates of Tables 2 to 5. An advantageous feature of the structural data according to Tables 2 to 5 are that they have a high resolution of from about 2.3 Å to 2.8 Å.

Indeed a further aspect of the invention includes within its scope a crystal of protein kinase Bβ (PKBβ) defined by structural data having a resolution of from about 2.3 Å to 2.8 Å, in particular 2.8 Å, 2.7 Å, 2.5 Å or 2.3 Å.

The coordinates of Tables 2 to 5 provide a measure of atomic location in Angstroms. The coordinates are a relative set of positions that define a shape in three dimensions, so the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 1.5 Å (preferably less than 1.0 Å and more preferably less than 0.5 Å) when superimposed on the coordinates provided for the residue backbone atoms, will generally result in a structure which is substantially the same as the structure of Tables 2 to 5 in terms of both its structural characteristics and usefulness for structure-based analysis, including design of PKBβ modulators.

Likewise the skilled person would understand that changing the number and/or positions of the water molecules in these structures (where shown) will not generally affect the usefulness of the structure for structure-based analysis. Thus for the purposes described herein as being aspects of the present invention, it is within the scope of the invention if: the coordinates are transposed to a different origin and/or axes; the relative atomic positions of the atoms of the structure are varied so that the root mean square deviation of residue backbone atoms is less than 1.5 Å (preferably less than 1.0 Å and more preferably less than 0.5 Å) when superimposed on the coordinates provided in Tables 2 to 5 for the residue backbone atoms. Reference herein to the coordinate data of Tables 2 to 5 thus includes the coordinate data in which one or more individual values of the Tables are varied in this way.

Modifications in the native PKBβ crystal structure due to e.g. mutations, additions, substitutions, and/or deletions of amino acid residues could lead to variations in the PKBβ atomic coordinates and where such modified forms of PKBβ are being investigated, atomic coordinate data of PKBβ modified so that a ligand that bound to one or more binding sites of PKBβ would be expected to bind to the corresponding binding sites of the modified PKBβ are, for the purposes described herein as being aspects of the present invention, also within the scope of the invention. Reference herein to the coordinates of Tables 2 to 5 thus includes the coordinates modified in this way. Preferably, the modified coordinate data define at least one PKBβ binding site.

In a further aspect, the invention provides a method for crystallizing a PKB derivative which comprises producing PKB by recombinant production in a host cell, recovering a PKB derivative from the host cell and growing one or more crystals from the recovered PKB derivative, wherein the PKB derivative is a stable protease-resistant form of PKB. The host cell may be of any suitable cell type, for example a eukaryotic cell host, such as a yeast cell, a mammalian cell, or an insect cell. In a preferred embodiment, the host cell is an insect cell, such as an Sf9 cell.

Typically the derivative lacks all or substantially all of the PH domain. Thus the derivative may be a truncated derivative e.g. truncated to positions 146-460 for PKBβ, or corresponding residues in other isoforms. The derivative may optionally include amino acid residues C-terminal of position 460 in PKBβ or its equivalent, e.g. the C-terminal 21 amino acids of PKBβ.

The method may further comprise the steps of phosphorylating one or more phosphorylatable residues in vitro with a suitable kinase. For example, PDK1 can be used to phosphorylate Thr-309 in vitro. It has been suggested that MAPKAP 2 kinase can be used to phosphorylate Ser-474 of PKBβ/Ser-473 of PKBα (Alessi et al. 1996).

Alternatively, the method may comprise the step of dephosphorylation in vitro, to ensure that any adventitious phosphorylation occurring during expression is removed. Numerous suitable enzymes will be known to the skilled person, e.g. the λ protein phosphatase.

The derivative may be encoded by a vector construct substantially similar to one disclosed herein. The method may include the further step of X-ray diffraction analysis of the obtained crystal.

Thus, the PKBβ produced by crystallising PKBβ (see the detailed description below) is provided as a crystallised protein suitable for X-ray diffraction analysis.

The crystal may be grown by any suitable method, e.g. the under oil batch method as described in the Examples.

The present invention further provides a recombinant polypeptide comprising the catalytic domain of PKB, the N-terminus of said polypeptide corresponding to Lys-146 of human PKBβ. The polypeptide will typically comprise the full kinase domain which may correspond, for example to amino acid residues 144 to 439 of human PKBα, 146 to 440 of human PKBβ, or 143 to 436 of human PKBγ.

In a preferred embodiment the polypeptide comprises amino acids 146 to 460 of human PKBβ, which corresponds to residues 145-459 of PKBα, and 143-456 of PKBγ. It may optionally further comprise the C-terminal region corresponding to amino acids 461 to 481 of human PKBβ or a portion thereof.

Reference to a PKB catalytic domain should be taken to include catalytic domains of mutant PKBs as described below (under ‘Homology Modelling’). The term ‘catalytic domain’ as used herein refers to the structural domain of the protein and should not be interpreted as requiring the polypeptide to have catalytic activity; for example it may contain a mutation which impairs or abrogates activity, e.g. at the active site, but which does not affect the gross structure of the domain.

The present invention further provides a crystallisable composition comprising a recombinant polypeptide as described above.

In a further aspect, the present invention provides nucleic acids encoding the polypeptides as described herein. Thus in these nucleic acids, the sequences encoding the catalytic domain are not contiguous with sequences encoding the PH domain of PKB, preferably not contiguous with sequences coding for any amino acids N-terminal of Lys-146.

The present invention also encompasses a method of making a polypeptide as disclosed, the method including the step of expressing said polypeptide or peptide from nucleic acid encoding it, which in most embodiments will be nucleic acid according to the present invention.

In another aspect, the invention provides a method of analysing a PKBβ-ligand complex comprising the step of employing (i) X-ray crystallographic diffraction data from the PKBβ-ligand complex and (ii) a three-dimensional structure of PKBβ to generate a difference Fourier electron density map of the complex, the three-dimensional structure being defined by atomic coordinate data according to Tables 2 to 5. If the PKBβ-ligand complex is crystallised in a different space group to the crystals described herein, molecular replacement methods may be used instead of difference Fourier methods.

Therefore, in the light of the present disclosure, PKBβ-ligand complexes can be crystallised and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallised PKBβ and the solved structure of un-complexed PKBβ. These maps can then be used to determine whether and where a particular ligand binds to PKBβ and/or changes the conformation of PKBβ.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualisation and model building programs such as O (Jones et al., Acta Crystallography, A47, (1991), 110-119) can be used.

In another aspect, the invention relates to methods of determining three dimensional structures of target kinases of unknown structure by utilising in whole or in part the structural coordinates provided for PKBβ in any one of the data sets provided herein (Tables 2 to 5).

The target kinase will typically be homologous to PKB, such as an AGC family kinase(e.g. SGK) (Hanks and Hunter (1995) FASEB J. 9: 576; Hardie, G. and Hanks, S. (eds) The Protein Kinase Facts Book—Protein Serine Kinases (1995) Academic Press Ltd., London). In particular, it may be an isoform of PKB, such as PKBα or PKBγ. The data provided here relate to the inactive conformation of PKBβ, and so will be useful for determining the structure of the corresponding conformation of other kinases. However, the present invention also extends to the elucidation of the structure of alternative conformations such as active conformations of such target kinases, including PKB, and including the active conformation of PKBβ, or PKB-ligand complexes.

There are three primary ways in which the three-dimensional coordinate data of the present invention can be used to solve other target kinase structures.

Firstly, the three-dimensional coordinate data provided herein for PKB may be aligned with an amino acid sequence of a target kinase to match homologous regions of the amino acid sequences, and a structure determined for the target kinase by homology modelling.

Secondly, the three-dimensional coordinate data of the present invention may be used to align the amino acid sequence of PKB with a known three-dimensional structure of a template kinase, in order to establish a target PKB structure; for example a structure for an alternative PKB conformation, such as an active PKB conformation

Thirdly, the three-dimensional coordinate data of the present invention may be used to assist in interpretation of a set of raw X-ray crystallographic data obtained for a target kinase, in order to establish a structure for the target kinase.

Typically, in each of these alternatives, the target structure will be established by the calculation of a set of three-dimensional coordinate data for some or all of the atoms in the target structure.

Homology Modelling

Thus the invention provides a method of homology modelling comprising the steps of:

(a) aligning a representation of an amino acid sequence of a target kinase of unknown structure with the amino acid sequence of PKBβ to match homologous regions of the amino acid sequences;

(b) modelling the structure of the matched homologous regions of the target kinase on the structure as defined by Tables 2 to 5 of the corresponding regions of PKBβ; and

(c) determining a conformation (e.g. so that favourable interactions are formed within the target kinase and/or so that a low energy conformation is formed) for the target kinase which substantially preserves the structure of said matched homologous regions.

The target kinase will typically be a PKB homologue, such as a member of the AGC kinase family. In particular, such a method may be used to determine the structure of the α isoform, γ isoform, or other isoforms of PKB or of related kinases such as the AGC kinase family.

The term “homologous regions” describes amino acid residues in two sequences that are identical or have similar (e.g. aliphatic, aromatic, polar, negatively charged, or positively charged) side-chain chemical groups. Identical and similar residues in homologous regions are sometimes described as being,respectively “invariant” and “conserved” by those skilled in the art.

Preferably one or all of steps (a) to (c) are performed by computer modelling.

Homology modelling is a technique that is well known to those skilled in the art (see e.g. Greer, Science, Vol. 228, (1985), 1055, and Blundell et al., Eur. J. Biochem, Vol. 172, (1988), 513). By “homology modelling”, is meant the prediction of related kinase structures based either on x-ray crystallographic data or computer-assisted de novo prediction of structure, based upon manipulation of the coordinate data of Tables 2 to 5.

The various in silico modelling techniques described in this section and in the other sections of this application may utilize coordinates from any of the four crystal data sets provided herein, or from any structure calculated by means of those data sets. To avoid unnecessary repetition, reference is made herein to the coordinate data of Tables 2 to 5.

“Homology modelling” extends to target kinases, in particular AGC kinases, which are analogues or homologues of the PKB protein whose structure has been determined in the accompanying examples. It also extends to mutants of PKB protein itself.

In general, comparison of amino acid sequences is accomplished by aligning the amino acid sequence of a polypeptide of a known structure with the amino acid sequence of the polypeptide of unknown structure. Amino acids in the sequences are then compared and groups of amino acids that are homologous are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST and BLAST 2 sequences (provided by the National Center for Biotechnology Information) are widely used in the art for this purpose, and can align homologous regions of two amino acid sequences. These may be used with default parameters to determine the degree of homology between the amino acid sequence of the protein of known structure and other target proteins which are to be modeled.

Analogues are defined as proteins with similar three-dimensional structures and/or functions and little evidence of a common ancestor at a sequence level.

Homologues are defined as proteins with evidence of a common ancestor i.e. likely to be the result of evolutionary divergence and are divided into remote, medium and close sub-divisions based on the degree (usually expressed as a percentage) of sequence identity.

A homologue is defined here as a protein with at least 15% sequence identity or which has at least one functional domain, which is characteristic of PKB. This includes polymorphic forms of PKB.

There are two types of homologue: orthologues and paralogues. Orthologues are defined as homologous genes in different organisms, i.e. the genes share a common ancestor coincident with the speciation event that generated them. Paralogues are defined as homologous genes in the same organism derived from a gene/chromosome/genome duplication, i.e. the common ancestor of the genes occurred since the last speciation event.

A mutant is a kinase characterized by replacement or deletion of at least one amino acid from a wild type AGC kinase, e.g. PKB. Such a mutant may be prepared for example by site-specific mutagenesis, or incorporation of natural or unnatural amino acids.

The present invention contemplates “mutants”, and the application of the methods of the present invention to “mutants”, wherein a “mutant” refers to a polypeptide which is obtained by replacing at least one amino acid residue in a native or synthetic ACG kinase with a different amino acid residue and/or by adding and/or deleting amino acid residues within the native polypeptide or at the N- and/or C-terminus of a polypeptide corresponding to a wild-type kinase and which has substantially the same three-dimensional structure as the kinase from which it is derived. By having substantially the same three-dimensional structure is meant having a set of atomic structure co-ordinates that have a root mean square deviation (r.m.s.d.) of less than or equal to about 2.0 Å when superimposed with the atomic structure co-ordinates of the wild-type kinase from which the mutant is derived when at least about 50% to 100% of the C_{α}atoms of the kinase are included in the superposition. A mutant may have, but need not have, enzymatic or catalytic activity.

To produce homologues or mutants, amino acids present in the said protein can be replaced by other amino acids having similar properties, for example hydrophobicity, hydrophobic moment, antigenicity, propensity to form or break α-helical or β-sheet structures, and so. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, an MBP tag, and epitope tags.

Amino acid substitutions, deletions and additions which do not significantly interfere with the three-dimensional structure of the kinase will depend, in part, on the region of the molecule where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a particular binding site or catalytic residue, in order to provide convenient cloning sites in cDNA encoding the polypeptide, to aid in purification of the polypeptide, etc. Such substitutions, deletions and/or additions which do not substantially alter the three dimensional structure of the wild-type kinase will be apparent to those having skills in the art.

It should be noted that the mutants contemplated herein need not exhibit enzymatic activity. Indeed, amino acid substitutions, additions or deletions that interfere with the catalytic activity of the kinase but which do not significantly alter the three-dimensional structure of the catalytic region are specifically contemplated by the invention. Such crystalline polypeptides, or the atomic structure co-ordinates obtained therefrom, can be used to identify compounds that bind to the protein.

Once the amino acid sequences of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure.

The structures of amino acids located in non-conserved regions may be assigned manually by using standard peptide geometries or by molecular simulation techniques, such as molecular dynamics. The final step in the process is accomplished by refining the entire structure using molecular dynamics and/or energy minimization.

Determination of Alternative Conformations of PKB

The present invention further provides a method for determining three-dimensional atomic coordinate data for a target conformation of a PKB isoform, comprising the steps of:

(a) employing three-dimensional atomic coordinate data of Tables 2 to 5;

(b) employing three-dimensional atomic coordinate data of a template kinase structure, and:

(c) determining three-dimensional atomic coordinate data for said target conformation.

The template kinase will typically be a homologue of PKB, such as an AGC family kinase. For example, the structure of the ternary complex of mouse PKA in its active conformation is known (Knighton et al., 1991a, b), so these coordinates would be suitable for this purpose.

It may be possible to estimate a structure for the target conformation of PKB by the homology modelling procedures outlined above, with reference only to the amino acid sequence of PKB. However, the three-dimensional coordinate data provided in Tables 2 to 5 shows features which cannot be determined from the sequence alone, such as the conformations and orientations of the amino acid side chains in the inactive conformation of PKB. Thus use of the PKB structural data provided here will provide a far more accurate structure for the target conformation than could be obtained simply by use of sequence data.

This aspect of the invention is applicable to the determination of structures for PKB isoforms other than the β isoform, e.g the α and γ isoforms, and mutants thereof as described above, using e.g. the mouse PKA structure as a template.

Structure Solution

In a further aspect, the invention provides a method for determining the structure of a target kinase, which method comprises;

providing the co-ordinates of Tables 2 to 5, and positioning the co-ordinates in the crystal unit cell of said target kinase so as to provide a structure for said target kinase.

In a preferred aspect of this invention the co-ordinates are used to solve the structure of target kinases particularly homologues of PKB, such as AGC family kinases, including, without limitation, NDR, p70 S6K, p90, PKC, etc..

The structures of the human PKB provided can be used to solve the crystal structure of other target AGC kinases including other crystal forms of PKB, mutants, and co-complexes of PKB, where X-ray diffraction data of these target proteins has been generated and requires interpretation in order to provide the structure.

In the case of PKB, this protein may crystallize in more than one crystal form. The structure coordinates of PKB, or portions thereof, as provided by this invention are particularly useful to solve the structure of those other crystal forms of PKB, such as that of the active conformation. They may also be used to solve the structure of PKB mutants or co-complexes, or of the crystalline form of any other protein with significant amino acid sequence homology to any functional domain of PKB, such as an AGC kinase family member.

In the case of other target proteins, particularly the AGC kinases referred to above, the present invention allows the structures of such targets to be obtained more readily where raw X-ray diffraction data is generated.

Thus, where X-ray crystallographic or NMR spectroscopic data is provided for a target kinase of unknown three-dimensional structure, the structure of PKB as defined by Tables 2 to 5 may be used to interpret that data to provide a likely structure for the other kinase by techniques which are well known in the art, e.g. phasing in the case of X-ray crystallography and assisting peak assignments in NMR spectra.

One method that may be employed for these purposes is molecular replacement. In this method, the unknown crystal structure, whether it is another crystal form of PKB, a mutant or co-complex thereof, or the crystal of a target kinase with amino acid sequence homology to any functional domain of PKB, may be determined using any one of the data sets of PKB structure coordinates of this invention as provided herein. This method will provide an accurate structural form for the unknown crystal more quickly and efficiently than attempting to determine such information ab initio.

Examples of computer programs known in the art for performing molecular replacement are CNX (Brunger A. T.; Adams P. D.; Rice L. M., Current Opinion in Structural Biology, Volume 8, Issue 5, October 1998, Pages 606-611 (also commercially available from Accelerys San Diego, Calif.) or AMORE (Navaza, J. (1994). AMoRe: an automated package for molecular replacement. Acta Cryst. ASO, 157-163).

The invention may also be used to assign peaks of NMR spectra of such proteins, by manipulation of the data provided herein.

Computer Systems

In another aspect, the present invention provides systems, particularly a computer system, intended to generate structures and/or perform rational drug design for PKBβ, PKBβ-ligand complexes or PKBβ homologues or mutants, the system containing either (a) atomic coordinate data according to Tables 2 to 5 recorded thereon, said data defining the three-dimensional structure of PKB, or at least selected coordinates thereof; (b) structure factor data for PKB recorded thereon, the structure factor data being derivable from the atomic coordinate data of Tables 2 to 5; (c) a Fourier transform of atomic coordinate data according to Tables 2 to 5, or at least selected coordinates thereof; (d) atomic coordinate data of a target kinase generated by homology modelling of the target based on the data of Tables 2 to 5; (e) atomic coordinate data of a target kinase generated by interpreting X-ray crystallographic data or NMR data by reference to the data of Tables 2 to 5; or (f) structure factor data derivable from the atomic coordinate data of (d) or (e).

The invention also provides such systems containing atomic coordinate data of target kinases wherein such data has been generated according to the methods of the invention described herein based on the starting data provided by Tables 2 to 5.

Such data is useful for a number of purposes, including the generation of structures to analyze the mechanisms of action of kinases, and/or to perform rational drug design of compounds which interact with them, such as modulators of kinase activity, e.g. activators or inhibitors.

In a further aspect, the present invention provides computer readable media with either (a) atomic coordinate data according to Tables 2 to 5 recorded thereon, said data defining the three-dimensional structure of PKB, or at least selected coordinates thereof; (b) structure factor data for PKB recorded thereon, the structure factor data being derivable from the atomic coordinate data of Tables 2 to 5; (c) a Fourier transform of atomic coordinate data according to Tables 2 to 5, or at least selected coordinates thereof; (d) atomic coordinate data of a target kinase generated by homology modelling of the target based on the data of Tables 2 to 5; (e) atomic coordinate data of a target kinase generated by interpreting X-ray crystallographic data or NMR data by reference to the data of Tables 2 to 5; or (f) structure factor data derivable from the atomic coordinate data of (d) or (e).

By providing such computer readable media, the atomic coordinate data can be routinely accessed to model PKB or selected coordinates thereof. For example, RASMOL (Sayle et al., TIBS, Vol. 20, (1995), 374) is a publicly available computer software package which allows access and analysis of atomic coordinate data for structure determination and/or rational drug design.

On the other hand, structure factor data, which are derivable from atomic coordinate data (see e.g. Blundell et al., in Protein Crystallography, Academic Press, New York, London and San Francisco, (1976)), are particularly useful for calculating e.g. difference Fourier electron density maps.

Uses of the Structures of the Invention

In another aspect, the present invention provides methods for modelling the interactions between PKB and modulators of PKB activity. Thus there is provided a method for modelling the interaction between PKB and an agent compound which modulates PKB activity, comprising the steps of:

(a) employing three-dimensional atomic coordinate data according to any one of Tables 2 to 5 to characterise at least one PKBβ binding site;

(b) providing the structure of said agent compound; and

(c) fitting said agent compound to the binding site.

The agent compound may be any compound known to have an effect on PKB activity, such as the peptide activating agents, e.g. PIFtide, described below.

The present invention further provides a method for identifying an agent compound (e.g. an inhibitor) which modulates PKB (e.g. PKBβ) activity, comprising the steps of:

(a) employing three-dimensional atomic coordinate data according to Tables 2 to 5 to characterise at least one PKBβ binding site;

(b) providing the structure of a candidate agent compound;

(c) fitting the candidate agent compound to the binding sites; and

(d) selecting the candidate agent compound.

Preferably a plurality of binding sites are characterised; preferably sufficient binding sites are characterised to define a PKBβ binding cavity and/or the ATP binding site which forms part of the catalytic site.

For ease of reference, and to avoid unnecessary repetition, only the production of modulators of PKB activity is discussed here. However the present invention is considered to apply equally to the identification of modulators of any target enzyme whose structure has been determined by reference to the three-dimensional coordinate data for PKBβ provided herein. For example, the data provided herein may be used to calculate a structure for a related AGC family kinase, such as (without limitation) SGK, p70 S6K, p90 RSK, PKC, and NDR. Accordingly, the present invention extends to the use of such a structure for identification of modulators of that target enzyme.

As discussed above, the data provided herein may enable the calculation of a structure for an active conformation of a PKB enzyme, such as PKBβ. This may prove particularly useful, because the structures described herein show that in the inactive conformation of PKBβ, the adenine moiety of ATP is prevented from binding to the ATP binding site by Phe-294. Since all known small molecule inhibitors of protein kinases are competitive with ATP, and therefore interact with the ATP binding site, an understanding of the PKB residues involved in the interaction with ATP allows the development of specific and potent inhibitors of this kinase. This information may thus be used to develop potent and specific small molecule inhibitors of PKB in a number of ways. PKBβ may be co-crystallised, and/or existing PKBβ crystals may be soaked, for example with known inhibitors of PKB, such as staurosporine, or those discovered in high-throughput screening programmes known to the skilled person. Alternatively, or additionally, rational drug design programmes may make full use of the crystallographic coordinates. These techniques are discussed in more detail below.

However, it may equally be possible to use the data provided herein to identify a modulator which binds to the inactive conformation of PKB and so stabilises it, inhibiting the transition to the active conformation. By analogy, STI571 is an inhibitor which binds to the ATP binding site of Abl tyrosine kinase but stabilises the inactive conformation of the enzyme.

It may be desirable to compare the structures of the inactive and active conformations of the enzyme, in order to identify binding sites present on only one of said conformations. The three-dimensional coordinate data for such a site could then be used to identify a ligand capable of binding selectively to, and stabilising, that conformation.

A plurality (for example two, three or four) of spaced PKBβ binding sites may be characterised and a plurality of respective compounds designed or selected. The agent compound may then be formed by linking the respective compounds into a larger compound which maintains the relative positions and orientations of the respective compounds at the binding sites. The larger compound may be formed as a real molecule or by computer modelling.

In any event, the determination of the three-dimensional structure of PKBβ provides a basis for the identification of new and specific ligands for PKB e.g. PKBβ, and other members of the AGC family of kinases, e.g. NDR, p70 S6K, p90, PKC, etc., for instance by computer modelling.

More specifically, a potential modulator of PKB activity can be examined through the use of computer modelling using a docking program such as GRAM, DOCK, or AUTODOCK (see Walters et al., Drug Discovery Today, Vol.3, No.4, (1998), 160-178, and Dunbrack et al., Folding and Design, 2, (1997), 27-42). This procedure can include computer fitting of candidate inhibitors to PKB to ascertain how well the shape and the chemical structure of the candidate inhibitor will bind to the enzyme.

Also computer-assisted, manual examination of the binding cavity structure of PKBβ may be performed. The use of programs such as GRID (Goodford, J. Med. Chem., 28, (1985), 849-857)—a program that determines probable interaction sites between molecules with various functional groups and the enzyme surface—may also be used to analyse the binding cavity to predict partial structures of inhibiting compounds.

Computer programs can be employed to estimate the attraction, repulsion, and steric hindrance of the two binding partners (e.g. the PKBβ and a candidate inhibitor). Generally the tighter the fit, the fewer the steric hindrances, and the greater the attractive forces, the more potent the potential modulator since these properties are consistent with a tighter binding constant. Furthermore, the more specificity in the design of a potential drug, the more likely it is that the drug will not interact with other proteins as well. This will tend to minimise potential side-effects due to unwanted interactions with other proteins.

In one embodiment a plurality of candidate agent compounds are screened or interrogated for interaction with the binding sites. In one example, step (b) involves providing the structures of the candidate agent compounds, each of which is then fitted in step (c) to computationally screen a database of compounds (such as the Cambridge Structural Database) for interaction with the binding sites. In another example, a 3-D descriptor for the agent compound is derived, the descriptor including e.g. geometric and functional constraints derived from the architecture and chemical nature of the binding cavity. The descriptor may then be used to interrogate the compound database, the identified agent compound being the compound which matches with the features of the descriptor. In effect, the descriptor is a type of virtual pharmacophore.

Having designed or selected possible binding partners, these can then be screened for activity. Consequently, the method preferably comprises the further steps of:

(e) obtaining or synthesising the candidate agent compound; and

(f) contacting the candidate agent compound with PKBβ to determine the ability of the candidate agent compound to interact with PKBβ (or similarly with other homologous isoforms or AGC kinase family members).

In step (f) the candidate agent compound may be contacted with PKBβ in the presence of a substrate, and typically a buffer, to determine the ability of the candidate agent compound to inhibit PKBβ. The buffer will typically contain ATP. The substrate may be e.g. a peptide corresponding to the sequence GRPRTTSFAE, or salts thereof. So, for example, an assay mixture for PKB may be produced which comprises the candidate inhibitor, substrate and buffer.

Instead of, or in addition to, performing e.g. a chemical assay, the method may comprise the further steps of:

(e) obtaining or synthesising the candidate agent compound;

(f) forming a complex of PKB and the candidate agent compound; and

(g) analysing (e.g. by the method of an earlier aspect of the invention) said complex by X-ray crystallography or NMR spectroscopy to determine the ability of the candidate agent compound to interact with PKB.

Detailed structural information can then be obtained about the binding of the agent compound to PKB, and in the light of this information adjustments can be made to the structure or functionality of the compound, e.g. to improve binding to the binding cavity. Steps (e) to (g) may be repeated and re-repeated as necessary. For X-ray crystallographic analysis, the complex may be formed by crystal soak-in methods or co-crystallisation, preferably co-crystallisation.

Greer et al. describes an iterative approach to ligand design based on repeated sequences of computer modelling, protein-ligand complex formation and X-ray crystallographic or NMR spectroscopic analysis. Thus novel thymidylate synthase inhibitor series were designed de novo by Greer et al., and PKB inhibitors may also be designed in the this way. More specifically, using e.g. GRID on the solved 3D structure of PKBβ, a ligand (e.g. a potential inhibitor) for PKB may be designed that complements the functionalities of the PKB binding site(s). The ligand can then be synthesised, formed into a complex with PKB or other AGC family kinase, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al., Medicinal Research Reviews, Vol.16, (1996), 3-50.

As a result of the determination of the PKBβ 3D structure, more purely computational techniques for rational drug design may also be used to design PKB modulators, e.g. activators or inhibitors (for an overview of these techniques see e.g. Walters et al.) . For example, automated ligand-receptor docking programs (discussed e.g. by Jones et al. in Current Opinion in Biotechnology, Vol.6, (1995), 652-656) which require accurate information on the atomic coordinates of target receptors may be used to design potential PKB modulators.

Linked-fragment approaches to drug design also require accurate information on the atomic coordinates of target receptors. The basic idea behind these approaches is to determine (computationally or experimentally) the binding locations of plural ligands to a target molecule, and then construct a molecular scaffold to connect the ligands together in such a way that their relative binding positions are preserved. The connected ligands thus form a potential lead compound that can be further refined using e.g. the iterative technique of Greer et al.. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design PKB inhibitors is made possible by the determination of the PKBβ structure.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al.) it is necessary to know beforehand the protein 3D structure (or at least the protein structure factors). Therefore, determination of the PKBβ structure also allows difference Fourier electron density maps of PKBβ-ligand complexes to be produced, which can greatly assist the process of rational drug design.

The approaches to structure-based drug design described above all require initial identification of possible compounds for interaction with the target bio-molecule (in this case PKB). Sometimes these compounds are known e.g. from the research literature.

Thus the present invention provides methods of identifying mimetics of known modulators of PKB activity. The methods may involve the identification of a binding site for the known modulator. Subsequently, candidate compounds may be fitted to the same binding site in order to identify a compound which will mimic the activity of the known modulator.

For example, the methods described above may be used to model the binding site at which PKB interacts with a known modulator, e.g. an activating agent such as PIFtide, as described elsewhere in this specification. A mimetic of the activating agent may then be designed by fitting candidate compounds to that binding site.

Thus the methods of the present invention for identifying agent compounds which modulate PKB activity may involve fitting a candidate agent compound to a PKB binding site, wherein the binding site has previously been determined to bind a known agent compound as described above.

When no suitable known starting compounds are known, or when novel compounds are wanted, a first stage of the drug design program may involve computer-based in silico screening of compound databases (such as the Cambridge Structural Database) with the aim of identifying compounds which interact with the binding site or sites of the target bio-molecule. Screening selection criteria may be based on pharmacokinetic properties such as metabolic stability and toxicity. However, determination of the PKBβ structure allows the architecture and chemical nature of each PKBβ binding site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

In another aspect, the invention includes a compound which is identified as a modulator of PKB activity by the method of the earlier aspect.

Following identification of a suitable modulator compound, it may be manufactured and/or used in the preparation, i.e. manufacture or formulation, of a composition such as a medicament, pharmaceutical composition or drug. These may be administered to individuals for treatment of an appropriate condition, e.g. inhibitors for use in the treatment of cancers, or activators in the use of diabetes, erectile dysfunction or neurodegeneration.

Thus, the present invention extends in various aspects not only to a modulator as provided by the invention, but also a pharmaceutical composition, medicament, drug or other composition comprising such a modulator e.g. for treatment (which may include preventative treatment) of disease such as cancer; a method comprising administration of such a composition to a patient, e.g. for treatment of disease such as cancer; use of such a modulator in the manufacture of a composition for administration, e.g. for treatment of disease such as cancer; and a method of making a pharmaceutical composition comprising admixing such a modulator with a pharmaceutically acceptable excipient, vehicle or carrier, and optionally other ingredients.

Activation of AGC Kinases

The insights into the mechanism of kinase activation, which the crystal structure of PKB provides, enables the provision of novel methods for activating AGC kinases, and materials for use it hose methods.

The present invention further provides a method of inducing a catalytic domain of an AGC kinase to adopt an active conformation, wherein the AGC kinase in its native form is regulated by phosphorylation of a regulatory phosphorylation site residue in a C-terminal regulatory segment distinct from said catalytic domain, said method comprising the steps of:

(a) providing a polypeptide comprising said catalytic domain, and

(b) forming a non-covalent complex between said polypeptide and an activating agent, wherein contact between said activating agent and said catalytic domain induces said catalytic domain to adopt an active conformation.

The activating agent does not catalyse covalent modification of the polypeptide; in particular, the activating agent is not a kinase and does not phosphorylate the polypeptide. Rather the activating agent interacts with the catalytic domain to induce ordering of the regions of the kinase corresponding to the αB and αC helices and activation segment of PKB. Full activity may also require phosphorylation of a residue in the activation segment corresponding to Thr-309 of human PKBβ. This disorder to order transition forms a hydrophobic surface groove in the N-terminal lobe of the catalytic domain which binds the activating agent. The interaction is believed to be stabilised further by electrostatic interactions between residues of the catalytic domain and one or more negative charges of the activating agent.

The catalytic domain may be that of any AGC kinase which, in its native form, is regulated by phosphorylation of a regulatory phosphorylation site residue in a C-terminal regulatory segment distinct from the catalytic domain. Such phosphorylation typically activates the kinase. Such kinases include, but are not limited to, PKB, PKC, NDR, SGK, and the p70 and p90 S6-kinases and include variants of these kinases which do not possess the regulatory phosphorylation site, such as the splice variant of PKBγ (Brodbeck et al., 2001). However, they do not include kinases which are not regulated by phosphorylation of this sort, such as PKA, PRK2, and PDK1.

By “AGC kinase” is meant any protein kinase which has a sequence identity of equal to or greater than 35% at the amino acid level with residues 37-350 of the catalytic subunit of PKA (Shoji et al., 1983). Determination of percentage sequence identity may be performed with the AMPS package as described by Barton (1994). AGC kinases are also described in detail by Hanks and Hunter FASEB J. (1995) 9: 576 and Hardie, G. and Hanks, S. (eds) The Protein Kinase Facts Book—Protein-Serine Kinases (1995) Academic Press Ltd., London).

Thus the kinases which can be activated by the methods of the present invention possess a regulatory segment distinct from the catalytic domain, which in PKB constitutes the portion of the protein C-terminal of the catalytic domain. Thus the term ‘C-terminal regulatory segment’ signifies only that this portion of the polypeptide is located C-terminal of the catalytic domain, and does not imply that any portion of the regulatory segment need form the C-terminus of the polypeptide. In a preferred embodiment, the C-terminal regulatory segment corresponds to amino acid residues 440 to 480 of PKBα, 441 to 481 of PKBβ, 438 to 479 of PKBγ, or corresponding residues in other kinases.

The regulatory segment contains a hydrophobic motif at least four amino acids and typically six amino acid residues in length, which typically contains the sequence FXXF, e.g. FXXFXY/F, although the kinase NDR has the sequence FXXY at this position. Here, and throughout this specification, X represents any amino acid. The regulatory segment further comprises a regulatory phosphorylation site, which typically lies within the hydrophobic motif, e.g. Ser-473 of PKBα, Ser-474 of PKBβ, Ser 472 of PKBγ. For example, PKBα, β and γ all have the sequence FPQFSY within their regulatory segment.

The term ‘catalytic domain’ as used herein refers to a protein domain which when folded has a particular characteristic structure, and not necessarily to a domain having any particular catalytic activity. Thus the catalytic domain may contain a mutation which impairs or abrogates activity, e.g. substitution or deletion of one or more amino acid residues at the active site, but which does not affect the gross structure of the folded domain.

The minimum catalytic domain of a given kinase is the minimum polypeptide sequence from that kinase which will fold stably into the appropriate conformation when expressed independently, and may correspond, for example to amino acid residues 144 to 439 of human PKBα, 146 to 440 of human PKBβ, or 143 to 436 of human PKBγ (see FIG. 7—all references made herein to numbering of residues of PKBα or β refer to the human PKB sequences as shown in FIG. 7).

Catalytic domains of other target AGC kinases may be identified by alignment of the target sequences with that of PKBβ.

In general, comparison of amino acid sequences is accomplished by aligning the amino acid sequence of a polypeptide of a known structure with the amino acid sequence of the polypeptide of unknown structure. Amino acids in the sequences are then compared and groups of amino acids that are homologous are grouped together. This method detects conserved regions of the polypeptides and accounts for amino acid insertions or deletions.

Homology between amino acid sequences can be determined using commercially available algorithms. The programs BLAST, gapped BLAST, BLASTN, PSI-BLAST and BLAST 2 sequences (provided by the National Center for Biotechnology Information) are widely used in the art for this purpose, and can align homologous regions of two amino acid sequences. These may be used with default parameters to determine the degree of homology between the amino acid sequence of the protein of known structure and those of target proteins.

The polypeptide may consist solely or essentially of the catalytic domain in isolated form, e.g. a recombinant single domain. Alternatively the polypeptide may contain further domains of the AGC kinase, fusion partners, epitope tags, etc. For example, the catalytic domain may be contiguous with all or part of one or more further domains found in the native wild-type form of the enzyme, such as a pleckstrin homology (PH) domain or the C-terminal regulatory segment of PKB.

In preferred embodiments, the catalytic domain is from an isoform of PKB, e.g. from the α, β or γ isoforms of PKB.

The catalytic domain may be provided in phosphorylated form, e.g. in the activation segment of the catalytic domain. For example, in a preferred embodiment the catalytic domain is from PKB and is provided phosphorylated at Thr-308 (PKBα), Thr-309 (PKBβ) or Thr-305 (PKBγ). In alternative embodiments where the catalytic domain is derived from another AGC kinase, it may be phosphorylated at the corresponding position.

The methods of the present invention may further comprise the steps of phosphorylating one or more phosphorylatable residues of the catalytic domain in vitro with a suitable kinase. For example, PDK1 can be used to phosphorylate Thr-309 in vitro, while it has been suggested that MAPKAP 2 kinase can be used to phosphorylate Ser-474 (Alessi et al., 1996).

Additionally or alternatively, the methods of the present invention may comprise the step of dephosphorylation in vitro, to ensure that any adventitious phosphorylation occurring during expression is removed. The skilled person will be aware of numerous suitable enzymes for this purpose, e.g. the λ protein phosphatase.

The activating agent may be a peptide. The peptide comprises an activation motif which is primarily responsible for mediating interaction with the catalytic domain. The activation motif may comprise a sequence derived from the native C-terminal regulatory segment of the same AGC kinase as the catalytic domain, or from the native C-terminal regulatory segment of a different AGC kinase, or may be a modified or mutated variant of either. Alternatively, the activation motif may be a synthetic sequence which does not occur naturally in an AGC kinase but which can activate the relevant catalytic domain in vitro, e.g. as described below.

The activation motif may comprise a hydrophobic motif. The hydrophobic motif is typically at least four amino acids in length, e.g. four, five or six amino acids in length, of which at least two amino acids, preferably at least three amino acids, are hydrophobic amino acids, preferably aromatic amino acids (e.g. phenylalanine, tyrosine). Preferably the hydrophobic motif comprises the sequence BXXB, where B represents an aromatic amino acid, e.g. tyrosine of phenylalanine and X is any amino acid. Thus in any sequence for an activating agent set out herein, it will be understood that phenylalanine can be replaced by tyrosine.

In a preferred embodiment, the hydrophobic motif comprises the sequence FXXF, YXXF, YXXY, FXXFX(Y/F), YXXFX(Y/F), or YXXYX(Y/F). In preferred embodiments, the hydrophobic motif comprises the sequence FXXFX(Y/F).

The activation motif preferably comprises an amino acid residue which carries a negative electrostatic charge at physiological pH. This amino acid may be located within, adjacent to or near (e.g. within one, two, three, four or five amino acids of) the hydrophobic motif, e.g. within the FXXF motif, or C-terminal of the FXXF motif, e.g. within one, two, three, four or five amino acids of the FXXF motif. The activation motif may comprise two such amino acids. In certain embodiments, one such amino acid may be located within the FXXF motif, and one may lie C terminal thereof, preferably one amino acid C-terminal thereof.

Preferably the activation motif comprises the sequence FXXFX′, FXXFX′(F/Y), FXX′FX′, or FXX′FX′(F/Y); YXXFX′, YXXFX′(F/Y), YXX′FX′, or YXX′FX′(F/Y); FXXYX′, FXXYX′(F/Y), FXX′YX′, or FXX′YX′(F/Y); YXXYX′, YXXYX′(F/Y), YXX′YX′, or YXX′YX′(F/Y); where X′ represents an amino acid residue which carries a negative charge at physiological pH. This may be a naturally ionisable acidic amino acid, such as aspartic acid or glutamic acid. Alternatively, X′ may be charged as a result of chemical derivatisation or enzymatic modification, e.g. it may be a phosphorylated amino acid residue, such as phosphoserine or phosphothreonine. Thus, particularly when X′ is phosphoserine or phosphothreonine, X′ may carry more than one negative charge at physiological pH.

In preferred embodiments, the activation motif comprises the sequence FXXFX′, FXXFX′(F/Y), FXX′FX′, or FXX′FX′(F/Y).

In preferred embodiments the activation motif is derived from the regulatory segment of PKB or PRK2. Preferably the activation motif comprises the sequence FPQFpSY (where pS is phosphoserine), FPQFDY or FRDFDY. For example, the activating agent may comprise the whole or part of one of the sequences GLLELDQRTHFPQFpSYSASIRE, GLLELDQRTHFPQFDYSASIRE and REPRILSEEEQEMFRDFDYIADWC (PIFtide—Biondi et al., 2000).

Activation of an AGC kinase according to the present invention may be performed in vivo or in vitro.

When performed in vitro, the methods of the present invention may be used, inter alia, to generate an active conformation of an AGC kinase catalytic domain for the purposes of structural analysis. Thus the present invention further provides a method of determining a structure for an active conformation of a catalytic domain of an AGC kinase, wherein the AGC kinase in its native form is regulated by phosphorylation of a regulatory phosphorylation site residue in a C-terminal regulatory segment, said method comprising the steps of inducing the catalytic domain of the AGC kinase to adopt an active conformation by any of the methods described herein.

The method may further comprise the step of obtaining a data set for said active conformation from which a structure can be calculated, and may additionally involve the step of calculating a structure therefor.

In preferred embodiments, especially where the active conformation is to be crystallised, a stable protease-resistant form of the catalytic domain is used, preferably in recombinant form. The catalytic domain may be a PKB catalytic domain, which may lack all or substantially all of the PH domain, e.g. corresponding to residues 1 to 139, 1 to 140, 1 to 141, 1 to 142, 1 to 143, 1 to 144, or 1 to 145 of human PKBβ, or their corresponding residues in other isoforms. In a preferred embodiment, the catalytic domain lacks residues corresponding to residues 1 to 145 of human PKBβ.

Additionally or alternatively the catalytic domain may be truncated at the C-terminus, e.g. lacking amino acid residues C-terminal of position 440 in PKBβ or its equivalent. In one embodiment, the catalytic domain lacks amino acid residues C-terminal of position 460 in PKBβ or its equivalent e.g. the C-terminal 21 amino acids of PKBβ. Thus it may be a truncated derivative of PKB, e.g. truncated to positions 146-460 for PKBβ, or corresponding residues in other isoforms.

The structure may be determined by any suitable method, e.g. X-ray crystallography or NMR. Thus the method may further comprise the step of crystallising the catalytic domain of the kinase in its active conformation.

The method may include the further step of X-ray diffraction analysis of the obtained crystal.

Alternatively, the methods of the present invention may be applied in assays for assessing the ability of a candidate agent to modulating the activity of an AGC kinase.

Thus the present invention further provides a method of assessing the ability of a candidate compound to modulate the catalytic activity of an AGC kinase, which in its native form is regulated by phosphorylation of a regulatory phosphorylation site residue in a C-terminal regulatory segment, comprising the steps of

(a) providing a polypeptide comprising a catalytic domain of said kinase,

(b) forming a non-covalent complex between said polypeptide and an activating agent, wherein contact between said activating agent and said catalytic domain induces said catalytic domain to adopt an active conformation, and

(c) contacting said non-covalent complex with said candidate agent.

The method may further comprise the step of measuring the effect of the candidate agent on the AGC kinase activity.

Preferably, the AGC kinase is phosphorylated at a position corresponding to Thr-309 of human PKBβ.

The methods may be used to identify modulators, such as inhibitors or activators of AGC kinases. Suitable methods for measuring the effect of candidate compounds on AGC kinase activity will be well known to the skilled person. For example, the activity of PKB can be assayed by monitoring phosphorylation of an appropriate substrate, e.g. the peptide Crosstide, as described in the Examples.

In a further aspect, the present invention provides a non-covalent complex between a catalytic domain of an AGC kinase, which in its native form is regulated by phosphorylation of a regulatory phosphorylation site residue in a C-terminal regulatory segment, and an activating agent, wherein said catalytic domain is in an active conformation, i.e. the regions of the catalytic domain corresponding to the αB and αC helices and activation segment of PKB are in an ordered conformation.

It will be clear from the above disclosure that a catalytic domain of an AGC kinase may also be induced to adopt an active conformation if covalently linked to an activating agent such as those described above.

Thus, the present invention also provides a method of inducing a catalytic domain of an AGC kinase to adopt an active conformation, wherein the AGC kinase in its native form is regulated by phosphorylation of a regulatory phosphorylation site residue in a C-terminal regulatory segment distinct from said catalytic domain, said method comprising the steps of:

(a) providing a polypeptide comprising said catalytic domain, and

(b) covalently joining said polypeptide to an activating agent, wherein contact between said activating agent and said catalytic domain induces said catalytic domain to adopt an active conformation.

Typically the polypeptide lacks some or all of a C-terminal regulatory domain prior to step (b). In preferred embodiments the polypeptide lacks the relevant regulatory phosphorylation site prior to step (b).

Preferably the activating agent is a peptide comprising an activation motif as described above, e.g. the peptide GLLELDQRTHFPQFDYSASIRE or REPRILSEEEQEMFRDFDYIADWC (PIFtide).

Ligation of a peptide to a polypeptide may be achieved by native chemical ligation, by protein splicing, or may be catalysed by a heterologous enzyme. Methods for carrying out such ligations are reviewed in Cotton, G. J. and Muir, T. W. (1999) Chemistry and Biology 6(9): R247-R256.

In preferred embodiments a phosphopeptide derived from the C-terminal regulatory segment of an AGC kinase is ligated to the catalytic domain. Preferably, the phosphopeptide is derived from the same AGC kinase as the catalytic domain. Thus this technique enables the active phosphorylated form of the enzyme to be mimicked without needing to phosphorylate the whole enzyme. This may be particularly useful where the kinase responsible for phosphorylation in vivo has not been conclusively identified.

In a preferred embodiment a polypeptide comprising a PKB catalytic domain is ligated to a peptide comprising the whole or part of the sequence GLLELDQRTHFPQFpSYSASIRE.

In a yet further aspect, the present invention provides a method of determining a structure for an active conformation of a catalytic domain of an AGC kinase, wherein the AGC kinase in its native form is regulated by phosphorylation of a regulatory phosphorylation site residue in a C-terminal regulatory segment distinct from said catalytic domain, said method comprising the steps of:

(a) providing a mutant AGC kinase protein comprising a catalytic domain and a C-terminal regulatory segment distinct from said catalytic domain, the protein further comprising a mutation which enhances the interaction between said regulatory segment and said catalytic domain relative to the wild type enzyme, such that an active conformation is induced in said catalytic domain, and

(b) obtaining a data set for said mutant protein from which a structure can be calculated.

The mutation enhances interaction between the regulatory segment (as described above) and the catalytic domain, such as to enable ordering of the regions of the kinase corresponding to the activation segment and αB and αC helices of PKB, without phosphorylation of a regulatory phosphorylation site in the C-terminal regulatory segment. The mutation may comprise one or more amino acid insertions, deletions or substitutions in the C-terminal regulatory segment, preferably in or around the hydrophobic motif, or in the catalytic domain, or in both C-terminal and catalytic domains. Alternatively, the mutation may involve the insertion or substitution of a number of contiguous residues of the C-terminal regulatory segment, e.g. with the corresponding residues from a second AGC kinase. Such a mutant AGC kinase may be considered to be a chimeric kinase.

Preferably, the C-terminal regulatory segment is mutated so that its interaction with the wild-type catalytic domain is enhanced. Preferably the mutation is made in or around the hydrohobic motif of the C-terminal regulatory segment, i.e. the region corresponding to the sequence FPQFSY of PKBβ (amino acid residues 470-475). The mutation may comprise substitution, deletion or insertion of one or more amino acids, e.g. 2, 3, 4, 5, 6, 7, 8, 9, 10, 15, or 20 amino acids. In preferred embodiments the regulatory phosphorylation site is mutated.

In preferred embodiments, the mutation comprises the introduction into the C-terminal regulatory segment of a residue which carries an electrostatic charge at physiological pH, preferably a negative electrostatic charge, e.g. aspartic acid or glutamic acid.

In a preferred embodiment the amino acid residue which would be phosphorylated to activate the wild-type enzyme (e.g. the residue corresponding to Ser-474 of PKBβ) is mutated to a residue which carries a negative electrostatic charge at physiological pH, e.g. aspartic acid or glutamic acid. For example, where the AGC kinase is PKBβ, the mutation may involve alteration of the sequence FPQFSY to FPQFDY.

In other embodiments, the mutation may involve the substitution of a number of contiguous residues of the C-terminal regulatory segment, e.g. with the corresponding residues from a second AGC kinase. Thus the sequence FPQFSY of PKBβ may be replaced by the sequence FRDFDY from PRK2. The chimera may contain further sequences from the second kinase, e.g. one or more of the flanking residues in the sequence GLLELDQRTHFPQFDYSASIRE from PKBβ may be replaced by one or more corresponding residues of the sequence REPRILSEEEQEMFRDFDYIADWC from PRK2 (PIFtide).

Additionally or alternatively, the catalytic domain may be mutated to enhance its interaction with the wild-type C-terminal regulatory segment, or with a mutated C-terminal regulatory segment. Thus the catalytic domain may be mutated in or around the binding groove which interacts with the C terminal regulatory segment. For example, polar or charged residues (e.g. serine, threonine, aspartic acid, glutamic acid, lysine, etc.) may be mutated to more hydrophobic residues (e.g. phenylalanine, tyrosine, etc.), or hydrophobic residues replaced by more hydrophobic or larger hydrophobic residues, in order to enhance the interaction between the catalytic domain and the hydrophobic motif of the regulatory segment.

Possible target residues include V194 and V198 of PKBβ. These may, for example, be replaced by the corresponding residues of the hydrophobic groove from PKA. This is capable of binding the regulatory segment of PKA without phosphorylation, which implies that the hydrophobic interactions involved are stronger than are seen in PKB. Thus possible substitutions include V194I and V198L.

Additionally or alternatively, substitutions may be made which enhance the binding of the catalytic domain to a negative charge of the regulatory segment, e.g. incorporating further positive charges. A possible target residue is S201; therefore a possible substitution is S201K.

Alternatively, both catalytic domain and C-terminal regulatory segment may be mutated in order to enhance the affinity between them. Mutants may be prepared for example, by site-specific mutagenesis, or incorporation of natural or unnatural amino acids.

In preferred embodiments, especially where the mutant AGC kinase is to be crystallised, a stable protease-resistant form of the catalytic domain truncated at the N-terminus is used. The kinase may lack some or all of the wild-type residues upstream of the catalytic domain, e.g. corresponding to all or substantially all of the PH domain of PKB, e.g. corresponding to residues 1 to 139, 1 to 140, 1 to 141, 1 to 142, 1 to 143, 1 to 144, 1 to 145, 1 to 146, 1 to 147, 1 to 148, 1 to 149 or 1 to 150 of human PKBβ, or their corresponding residues in other isoforms. In a preferred embodiment the kinase lacks residues corresponding to residues 1 to 145 of PKBβ.

In a further aspect, the present invention provides a mutant AGC kinase protein, wherein the AGC kinase in its native form is regulated by phosphorylation of a regulatory phosphorylation site residue in a C-terminal regulatory segment, said mutant AGC kinase protein comprising a catalytic domain and a C-terminal regulatory segment distinct from said catalytic domain, and having an N-terminus corresponding to residue 139, 140, 141, 142, 143, 144, 145, 146, 147, 148, 149 or 150 of PKBβ, or their corresponding residues in other isoforms, the mutant AGC kinase protein comprising a mutation which enhances the interaction between said regulatory segment and said catalytic domain relative to the wild type enzyme, such that an active conformation is induced in said catalytic domain.

The mutation enhances interaction between the regulatory segment and the catalytic domain, such as to enable ordering of the regions of the kinase corresponding to the activation segment and αB and αC helices of PKB, without phosphorylation of the regulatory segment, and may have any of the characteristics described above.

In a preferred embodiment the kinase has an N-terminus corresponding to residue 146 of PKBβ.

In a further aspect, the present invention provides nucleic acids encoding the mutant AGC kinase polypeptides as described herein.

Throughout this specification, where nucleic acids are referred to, they may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

Nucleic acid according to the present invention may be polynucleotides or oligonucleotides, and may include cDNA, RNA, genomic DNA (gDNA) and modified nucleic acids or nucleic acid analogs.

Where a nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby by each nucleotide is base paired to its counterpart i.e. G to C, and A to T or U.

The nucleic acids of the present invention may differ from any specific sequences recited or referred to herein by a change which is one or more of addition, insertion, deletion and substitution of one or more nucleotides of the sequences shown, e.g. 1, 2, 3, 4, 5, 6, 7, 8, 9, 10, 15, 20, 25, 30, 40, 50 or more nucleotides. Preferably the reading frame is maintained. Changes to a nucleotide sequence may result in an amino acid change at the protein level, or not, as determined by the degeneracy of the genetic code.

Nucleic acids of the present invention may be provided as part of a vector, and also provided by the present invention is a vector comprising nucleic acid as described herein, particularly vectors from which the polypeptide can be expressed under appropriate conditions, and a host cell containing any such vector or nucleic acid.

‘Vector’ is defined to include, inter alia, any virus, plasmid, cosmid, or phage vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, insect, yeast or fungal cells).

A vector including nucleic acid according to the present invention need not include a promoter or other regulatory sequence, particularly if the vector is to be used to introduce the nucleic acid into cells for recombination into the genome.

Preferably a nucleic acid sequence of the present invention in the vector is under the control of, and operably linked to, an appropriate promoter or other regulatory elements for transcription in a host cell such as a microbial, e.g. bacterial, or yeast cell, or an insect or mammalian cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell

By “promoter” is meant a sequence of nucleotides from which transcription may be initiated of DNA operably linked downstream (i.e. in the 3′ direction on the sense strand of double-stranded DNA).

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment, the promoter is an inducible proimoter The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus these aspects of the Invention provide a gene construct, preferably a replicable vector, comprising a promoter (optionally inducible) operably linked to a nucleotide sequence provided by the present invention.

Preferably the vector is capable of providing expression in an insect cell, such as an Sf9 cell, especially where the expressed product is to be crystallised. The polypeptide may be encoded by a vector construct substantially similar to those disclosed herein.

The present invention also encompasses method of making peptides or polypeptides as disclosed, the method including the step of expressing said polypeptide or peptide from nucleic acid encoding it, which in most embodiments will be nucleic acid according to the present invention. This may conveniently be achieved by growing a host cell containing such a vector in culture under appropriate conditions which cause or allow expression of the polypeptide. Polypeptides and peptides may also be expressed in in vitro systems, such as reticulocyte lysates, as will be appreciated by the skilled person.

Systems for cloning and expression of a polypeptide in a variety of different host cells are well known. Suitable host cells include bacteria, eukaryotic cells such as mammalian and yeast, and baculovirus-based insect expression systems. Mammalian cell lines available in the art for expression of a heterologous polypeptide include Chinese hamster ovary cells, HeLa cells, baby hamster kidney cells, COS cells and many others.

Although certain specific amino acid sequences are referred to herein, e.g. in the context of peptides capable of activating AGC kinases, it will be appreciated that similar sequences having functionally insignificant changes are equally appropriate for practising the present invention. Therefore amino acids present in the said sequences can be replaced by other amino acids having similar properties, for example hydrophobicity, hydrophobic moment, antigenicity, propensity to form or break α-helical or β-sheet structures, and so. Substitutional variants of a protein are those in which at least one amino acid in the protein sequence has been removed and a different residue inserted in its place. Amino acid substitutions are typically of single residues but may be clustered depending on functional constraints e.g. at a crystal contact. Preferably amino acid substitutions will comprise conservative amino acid substitutions. Insertional amino acid variants are those in which one or more amino acids are introduced. This can be amino-terminal and/or carboxy-terminal fusion as well as intrasequence. Examples of amino-terminal and/or carboxy-terminal fusions are affinity tags, maltose binding protein (MBP) tags, and epitope tags.

Amino acid substitutions, deletions and additions which do not significantly interfere with three-dimensional structure will depend, in part, on the region of the molecule where the substitution, addition or deletion occurs. In highly variable regions of the molecule, non-conservative substitutions as well as conservative substitutions may be tolerated without significantly disrupting the three-dimensional structure of the molecule. In highly conserved regions, or regions containing significant secondary structure, conservative amino acid substitutions are preferred.

Conservative amino acid substitutions are well-known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

In some instances, it may be particularly advantageous or convenient to substitute, delete and/or add amino acid residues to a particular binding site or catalytic residue, in order to provide convenient cloning sites in cDNA encoding the polypeptide, to aid in purification of the polypeptide, etc. Such substitutions, deletions and/or additions which do not substantially alter the three dimensional structure of the wild-type kinase will be apparent to those having skills in the art.

Particular embodiments of the invention will now be described, by way of example only, with reference to the accompanying drawings.

### BRIEF DESCRIPTION OF THE DRAWINGS

FIG. 1 shows a comparison of PKB and PKA structures, with ribbon representations of PKA (A) and PKB (B). PKA and PKB were superimposed onto their C-terminal lobes. Phe 294 of the DFG motif of PKB occupies a site equivalent to the adenine pocket of the nucleotide binding site of PKA. (C) Stereo view of a superimposition of PKA and PKB to show different relative orientations of their N- and C-terminal lobes. Conformational differences in C-lobe are localised to the activation segment and αF/αG loop. Figure drawn using BOBSCRIPT (Esnouf, 1997) and RASTER3D (Merit and Murphy, 1994)

FIG. 2 shows the structure of the N-terminal Lobe:

(A) Flexibility of αB- and αC-helices. 2Fo-Fc electron density map contoured at 1σ of a portion of the N-terminal lobe of pΔPH-PKB-ΔC (β3, β4, β5-strands, βB- and βC-helices). Electron density for the β-sheet is well resolved, whereas the αB- and αC-helices are disordered. The main-chain of the N-terminal lobe and hydrophobic motif of PKA is shown superimposed onto PKB.

(B and C) Role of hydrophobic motif to order the αB- and αC-helices and link to activation segment. (B) Interactions of hydrophobic motif of PKA with the β3, β, β5-strands and αB- and αC-helices of the N-terminal lobe. Phe 347 and Phe 350 are buried by hydrophobic residues. Glu 349 and C-terminal carboxylate form hydrogen bonds with basic residues of the αC-helix. (C) Disorder of the αB- and αC-helices of PKB is correlated with absence of bound hydrophobic motif. In (B) bracketed residues corresponds to PKB numbering.

FIG. 3. Role of αC-helix to regulate conformation of PKA and PKB and structure of activation segment and DFG motif. (A) αC-helix stabilises an active state of PKA by interaction with pThr 197 of the activation segment via His 87, and Phe 185 of the DFG motif via Ile 93 and Leu 94. (B) In PKB, disorder of the αC-helix prevents His 196 from interacting with pThr 309. Loss of interactions with Phe 294 of the DFG motif binds within the nucleotide-binding site of ATP.

FIG. 4 shows a multiple sequence alignment of the catalytic domains and C-terminal regulatory segments of various AGC-family protein kinases. Invariant residues are shown with dark shading and conserved residues with light shading. The position of critical functional residues are indicated with a dark arrow and numbered according to PKA residues. PKB Thr 309 and Ser 474 phosphorylation sites are indicated. The conserved AGC-kinase hydrophobic motif is shown and mutated residues of PKB that influence PIFtide activation (FIG. 7B) are indicated by light arrows. Figure drawn using ALSCRIPT (Barton, 1993).

FIG. 5 illustrates the activation of PKB by hydrophobic motif peptides and complex formation between PKB and PIFtide.

(A) Dose response curve for the activation of ΔPH-PKB-ΔC by various synthetic 23 residue peptides derived from the PKB regulatory segment. ●: PKB HM-P has a phosphoserine residue at position 474; ▴: PKB HM-D has aspartate at position 474; ∘: has an unphosphorylated serine residue at position 474 and so corresponds to the wild type sequence.

(B) Dose response curve for the activation of (p) ΔPH-PKB-ΔC by PIFtide a synthetic 24 residue peptide encompassing the PRK2 HM motif. ●: PIFtide and pΔPH-PKB-ΔC, ▴: PIFtide and ΔPH-PKB-ΔC, ∘: mutant PIFtide (D>A) and pΔPH-PKB-ΔC. PIFtide can bind to ΔPH-PKB-ΔC but cannot activate it in the absence of Thr-309 phosphorylation.

(C) Isothermal titration calorimetry measurements of the binding of PIFtide to pΔPH-PKB-ΔC (left) and ΔPH-PKB-ΔC (right). Upper panel, raw data of the titration of PIFtide into pΔPH-PKB-ΔC. Lower panel, integrated heats of injections, corrected for the heat of dilution, with the solid line corresponding to the best fit of the data using the MicroCal software.

FIG. 6 shows that conserved residues of the hydrophobic motif, and residues of the N-lobe of PKB, are required for PIFtide and PKB HM-peptide mediated stimulation of PKB kinase activity. Mutations of conserved hydrophobic motif residues of PIFtide and PKB HM-peptide reduce or eliminate their potential to activate ΔPH-PKB-ΔC phosphorylated on Thr 309. Mutations of hydrophobic and electrostatic residues of the ΔPH-PKB-ΔC N-lobe hydrophobic groove reduces the stimulation of PKB activity by 130 μM PIFtide. The position of mutated residues on PKA and PKB (R202D, V194A-V198A and L225A) are shown in FIG. 4.

FIG. 7 is a comparison of the amino acid sequences of human PKBα, PKBβ and PKBγ. The PH and catalytic domains are shown boxed, and are connected by the linker domain. The GXXGXG ATP binding site, the catalytic lysine residue, and the regulatory phosphorylation sites are shown in bold type.

### DETAILED DESCRIPTION OF THE INVENTION

Limited trypsinolysis of full length PKBβ purified from Sf9 cells led the present inventors to the identification of a protease resistant domain with an N-terminus at Lys-146, referred to as ΔPH-PKB. Lys-146 is located within the structurally diverse region linking the pleckstrin homology (PH) and kinase domains of PKB, close to the N-terminus of the corresponding β1-strand of PKA. During the course of the purification, partial cleavage of a C-terminal 3 kDa fragment was observed, suggesting conformational flexibility at the C-terminus of the protein. Human PKBα, PKBβ and PKBγ sequences are structurally diverse within a 12 residue region C-terminal to the conserved PP(D/E) motif (residues 452-454 of PKBβ), preceding the C-terminal hydrophobic motif, and corresponding to the C-terminus of the PKBβ splice variant.

Using this information, the present inventors constructed a number of new PKB baculovirus Fastbac entry vectors for the generation of PKB insect cell/baculovirus expression systems, and expressed the β and γ-isoforms of PKB as the kinase domain, with an N-terminus at Lys-146 (i.e. lacking the PH domain), with and without the C-terminal 21 residues that includes the hydrophobic regulatory segment. These two kinase domains are termed ΔPH-PKB and ΔPKB-ΔC, respectively.

These expression systems express high levels of protein, which have been purified to homogeneity. Moreover, PDK1 has been expressed using the insect cell/baculovirus system and MAPKAPK2 in the E. coli expression system to enable phosphorylation of PKBβ on Thr309 and Ser474, respectively.

To prepare defined phosphorylated states of PKB, phosphorylation and dephosphorylation reactions were performed using PDK1 (for pThr-309) and the non-specific λ-protein phosphatase, respectively. Distinct phosphorylated states of the protein were resolved using hydrophobic interaction chromatography.

The phosphorylation state of the protein was analysed by Western blots using phospho-specific antibodies, and the stoichiometry and sites of phosphorylation were quantitatively assessed by mass spectroscopic analysis of trypsin-generated peptides of the protein.

Crystals were successfully obtained for the PKBβ derivatives, and structures determined for ΔPH-PKBβ-ΔC, pΔPH-PKBβ-ΔC and ΔPH-PKBβ by X-ray crystallographic techniques. High resolution structures were obtained, apparently showing the catalytic domain of PKBβ in the inactive conformation.

The phosphorylation state of the protein was analysed by Western blots using phospho-specific antibodies, and the stoichiometry and sites of phosphorylation were quantitatively assessed by mass spectroscopic analysis of trypsin-generated peptides of the protein. The three crystal forms of human PKBβ are; (i) pΔPH-PKB-ΔC, phosphorylated in vitro on Thr-309, (ii) ΔPH-PKB-ΔC, not phosphorylated on Thr-309, and (iii) ΔPH-PKB-β, dephosphorylated in vitro.

Two different batches of crystals, having different resolution, were produced for crystal form (i), i.e. pΔPH-PKB-ΔC.

### RESULTS

Each of the crystals belonged to the tetragonal space group P4₁2₁2, and accommodated one molecule of PKB per asymmetric unit with cell parameters as follows:

pΔPH-PKB-ΔC (first batch): a=149.33 Å, b=149.33 Å, c=39.77 Å;

pΔPH-PKB-ΔC (second batch): a=148.40 Å, b=148.40 Å, c=38.55 Å;

ΔPH-PKB-ΔC: a=149.70 Å, b=149.70 Å, c=39.19 Å;

ΔPH-PKB: a=149.52 Å, b=149.52 Å, c=39.06.

Resolution was determined to be 2.8 Å for the first batch and 2.3 Å for the second batch of pΔPH-PKB-ΔC crystals, 2.7 Å for ΔPH-PKB-ΔC and 2.5 Å for ΔPH-PKB.

The current refined R-factor:

(Σ|F₀−F_{c}|/Σ|F₀|, where F₀=observed amplitude, and F_{c}=calculated amplitude) is as follows:

pΔPH-PKBβ-ΔC (second batch): 0.237 to 2.3 Å resolution.

ΔPH-PKBβ-ΔC: 0.238 to 2.7 Å resolution.

ΔPH-PKBβ: 0.254 to 2.5 Å resolution.

More detailed information about the data collection and refinement statistics are provided for all crystals except the first batch of pΔPH-PKB-ΔC is provided in Table 1 below.

TABLE 1
Crystallographic Data Collection and Refinement Statistics

| Protein | pΔPH-PKBβ-ΔC | ΔPH-PKBβ-ΔC | ΔPH-PKBβ |
|---|---|---|---|
| Amino acid residues | 146-460 | 146-460 | 146-481 |
| Phosphorylation | Thr-309 | — | — |
| Space group (Z) | P4₁212 (1) | P4₁212 (1) | P4₁212 (1) |
| Cell parameters a | 148.40 | 149.70 | 149.52 |
| (Å) | | | |
| c (Å) | 38.55 | 39.19 | 39.06 |
| X-ray source | ID14eh4 ESRF | ID14eh4 ESRF | ID14eh4 ESRF |
| Resolution (Å) | 2.3 | 2.7 | 2.5 |
| Observations (N) | 113 677 | 50 875 | 92 809 |
| Unique (N) | 18 905 | 12 147 | 16 090 |
| Completeness (%) | 96.1 | 94.2 | 99.7 |
| ^{a}R_{sym} | 0.050 (0.243) | 0.065 (0.236) | 0.057 |
| (0.255) | | | |
| I/σI | 21.0 | 18.0 | 14.8 |
| Refinement | | | |
| Resolution range | 35-2.3 | 35-2.75 | 35-2.6 |
| (Å) | | | |
| Reflections used (N) | 17 576 | 10 320 | 14 317 |
| R_{free} set (N) (%) | 1398 (7.1) | 1199 (9.9) | 1598 (10.0) |
| ^{b}R_{cryst}/R_{free} | 0.237/0.309 | 0.238/0.30 | 0.254/0.314 |
| Protein atoms (N) | 2 198 | 2 198 | 2198 |
| Solvent atoms (N) | 154 | 27 | 125 |
| r.m.s.d. bond angles | 1.54 | 1.57 | 1.53 |
| (°) | | | |
| r.m.s.d. bond | 0.0105 | 0.0112 | 0.0104 |
| lengths (Å) | | | |

Values in parentheses are for the highest shell ^{a}R_{sym}=ΣₕΣⱼ|<I(h)>-I (h)ⱼ|/ΣₕΣⱼ<I(h)>, where <I(h)> is the mean intensity of symmetry-equivalent reflections.

^{b}R_{cryst/free}=Σ||F_{obs}|−|F_{calc}||/Σ|F_{obs}|, where F_{obs} and F_{calc} are the observed and calculated structure factors, respectively.

Root-mean-square deviations relate to the Engh and Huber parameters.

Coordinate data for the four crystals is provided at the end of this specification as follows:

Table 2: pΔPH-PKBβ-ΔC (first batch)

Table 3: pΔPH-PKBβ-ΔC (second batch)

Table 4: ΔPH-PKBβ-ΔC

Table 5: ΔPH-PKBβ

Overall Description of the Structure and Comparison with PKA

The structure of pΔPH-PKB-ΔC is essentially identical to those of ΔPH-PKB-ΔC and ΔPH-PKB (rms deviations of 0.3 Å and 0.4 Å, respectively), and this similarity to inactive forms of PKB, together with features of the structure, indicates that the crystallisation conditions favoured the inactive conformation of pΔPH-PKB-ΔC. Because of the higher resolution of the pΔPH-PKB-ΔC crystal structure, most of the discussion is focussed on this form.

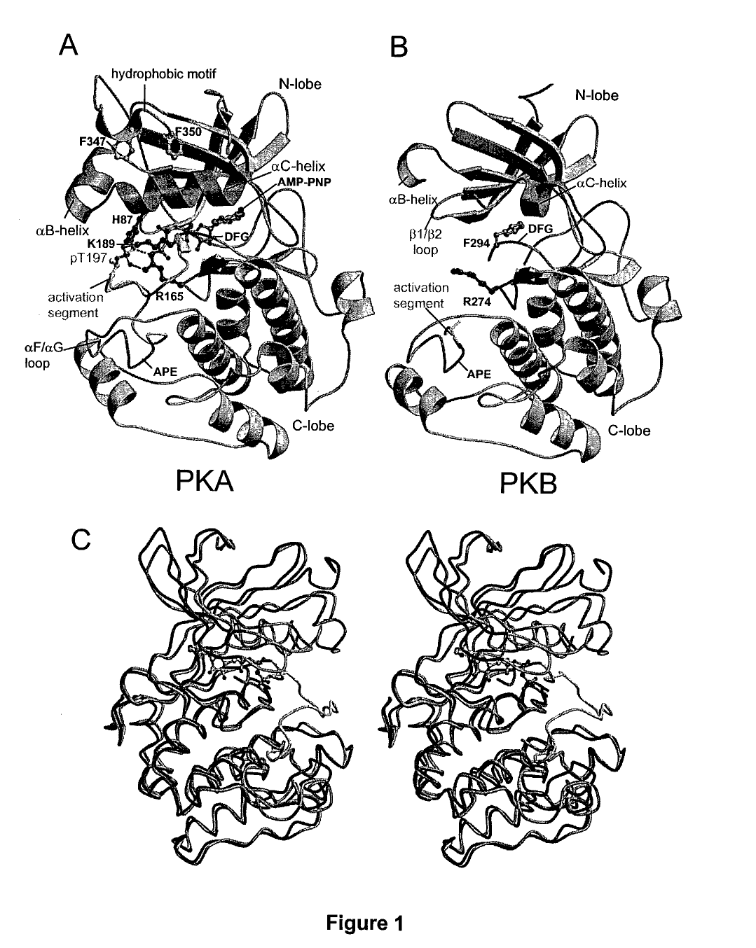

The structure of pΔPH-PKBβ-ΔC (residues 146-460) resembles the catalytic domain of other protein kinases (reviewed by Johnson et al., 1996). In particular, it resembles that of the catalytic subunit of PKA (FIG. 1). The PKB molecule is organised into an N-terminal and C-terminal lobe, with the N-terminal lobe (residues 146-233) formed from a 5-membered β-sheet and flanking α-helix, αA (equivalent to αC of PKA). The C-terminal lobe (residues 234-450) is predominantly α-helical and is joined to the N-terminal lobe via a single polypeptide chain connection.

The catalytic site of PKB is situated at the interface of the N and C-terminal lobes and is formed from residues of the catalytic loop (residues 274-282), and the activation segment (residues 304-312) of the C-terminal lobe, together with the ATP binding site and the αA helix of the N-terminal lobe. The ATP binding site consists of a hydrophobic pocket formed by residues (Val158, Val166) that interact with the adenine ring of the nucleotide, and a more hydrophilic region that interacts with the ribose ring and phosphate groups. By analogy with other protein kinases (Hubbard, 1997), the activation segment provides the binding site for the peptide substrate, orientating the substrate amino acid towards the phosphates of the ATP.

The catalytic mechanism of all protein kinases is similar and involves a phosphoryl transfer reaction from the γ-phosphate group of the ATP onto the hydroxyl group of the substrate amino acid residue. The reaction commences with the nucleophilic attack by the hydroxyl group of the substrate amino acid residues onto the γ-phosphate of ATP. A catalytic base in PKBβ, Asp265, facilitates this attack by increasing the nucleophilicity of the substrate hydroxyl group. The phosphate moieties of ATP are coordinated by the glycine rich loop and Lys181 of the N-terminal lobe and by a Mg²⁺ ion that interacts with Asp293 of the protein kinase C-terminal lobe.

PKA and PKB share essentially the same secondary structure topology, except that in PKB there is no counterpart to the αA-helix of PKA, and some of the structural elements of PKB are disordered. The architecture of PKA consists of an N-terminal lobe based on a 5-stranded β-sheet, with two α-helices (the αB- and αC-helices), and a larger, mainly α-helical C-terminal lobe, containing the activation segment. The catalytic site for ATP is located at the interface of the two lobes, whereas the substrate peptide-binding site is within the C-lobe, centred on the activation segment.

The inactive state of PKB differs in structure from the catalytically active form of PKA in a number of respects that are important for the regulation of PKB by multi-site phosphorylation. These differences involve the overall juxtaposition of the N- and C-lobes of the kinase, and structural disorder of the αB- and αC-helices of the N-lobe, activation segment of the C-lobe, and C-terminal regulatory segment. When superimposed, equivalent Cα-atoms of PKB and the ternary complex of PKA differ by an rms deviation of 2.3 Å (FIG. 1C). This deviation is larger than the expected value of 1.2 Å for a pair of proteins with 43% sequence identity (Cothia and Lesk, 1986), and results from differences in the relative orientations of the N- and C-terminal lobes of PKA and PKB. When superimposed individually, differences in conformation between the equivalent N- and C-lobes of PKA and PKB are seen to be localised to the β1-strand and αC-helix in the N-lobe, and DFG motif and αF/αG loop in the C-lobe.

PKA adopts open and closed conformational states resulting from relative rotations of the N- and C-lobes that are associated with various substrate-PKA complexes, with the ternary-PKA complex adopting a closed state, and the apo and binary complexes being more open. However, the relative position of the N- and C-lobes of PKB, do not resemble any of the various PKA-ligand complexes. Compared with the PKA-ternary complex, the N-lobe of PKB is rotated by 20° relative to its C-lobe, causing catalytic site residues from the two lobes to be misaligned.

Comparison of a number of activated protein kinase structures indicates that their N- and C-terminal lobes adopt similar relative configurations. For example, the Cα-Cα distance of two residues (Val-57 and Leu-173) that span the nucleotide-binding site in the PKA ternary complex is 12.9 Å, the same as that between equivalent residues of the phosphorylated insulin receptor kinase (Hubbard, 1997). In contrast, for PKB this distance is 15.2 Å.

Structural Disorder in PKB

In addition to variations in their overall bilobal configuration, the structures of PKB and PKA differ in other respects that are significant for the reduced catalytic activity of unphosphorylated and mono-phosphorylated forms of PKB. In the inactive PKB structures, three inter-related regions of the polypeptide chain are disordered; (i) the αB- and αC-helices of the N-terminal lobe, (ii) the activation segment between the invariant DFG and APE motifs, and (iii) the C-terminal regulatory segment in ΔPH-PKB. Concerted disorder to order transitions of these regions, linked to a conformational change of the activation segment DFG motif, and reorganisation of the N- and C-lobes, are required to generate a catalytically active protein kinase on phosphorylation of Thr-309 and Ser-474.

Although the structures of the three disordered regions are interdependent, each region is described in turn, before discussing the biological implications that these regions are disordered. This analysis is greatly assisted by the ability to compare the structural differences between an inactive PKB molecule with that of the related active PKA catalytic subunit, the latter serving as a model for an active phosphorylated form of PKB.

Flexibility of the αB- and αC-helices

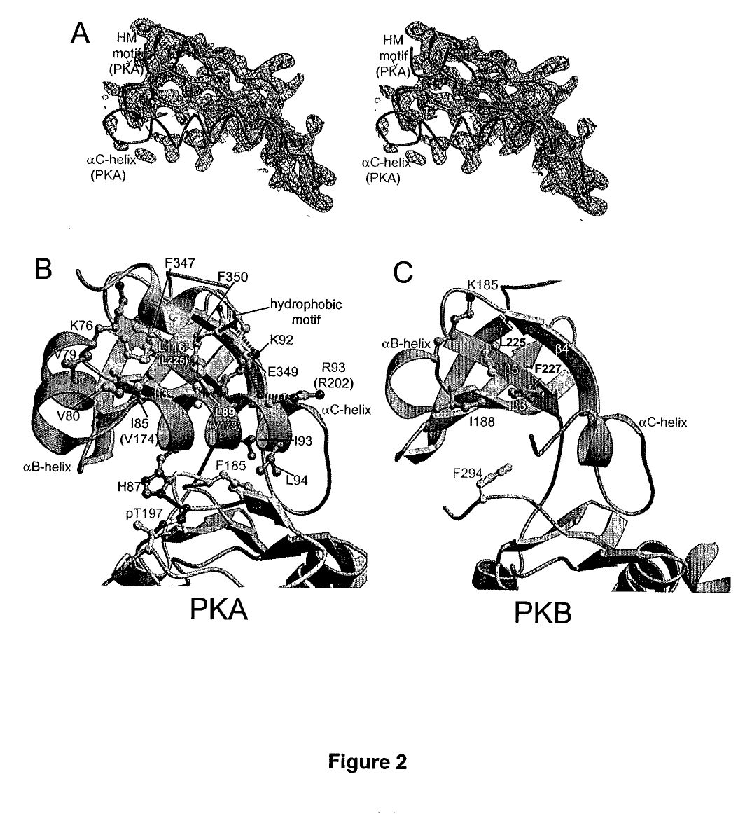

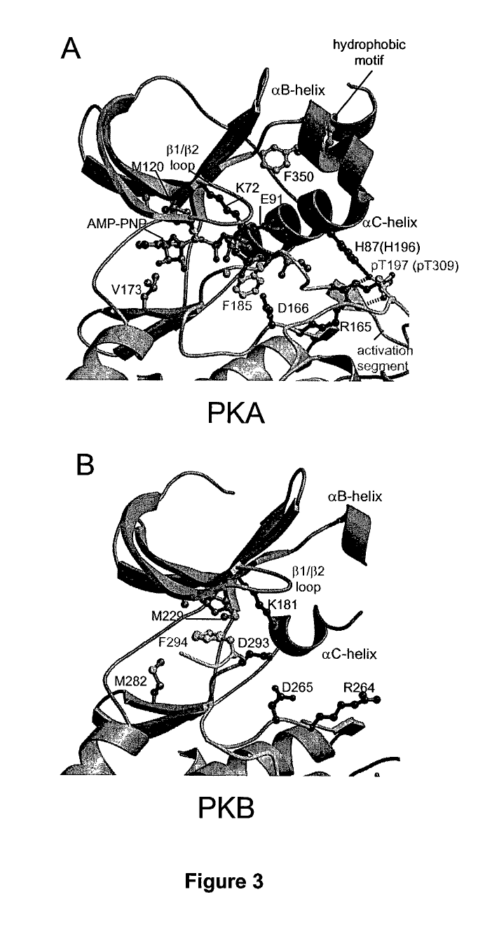

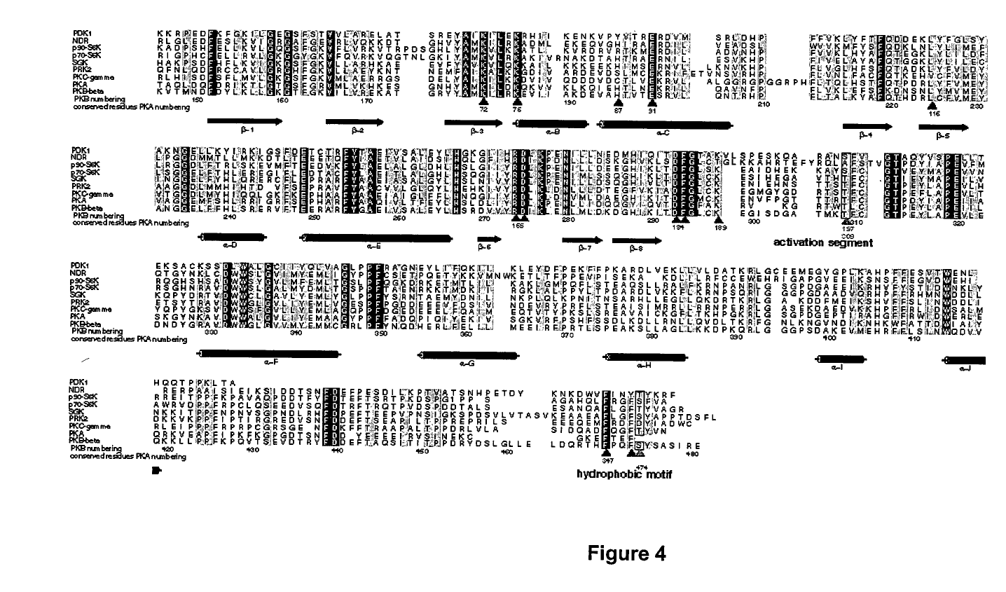

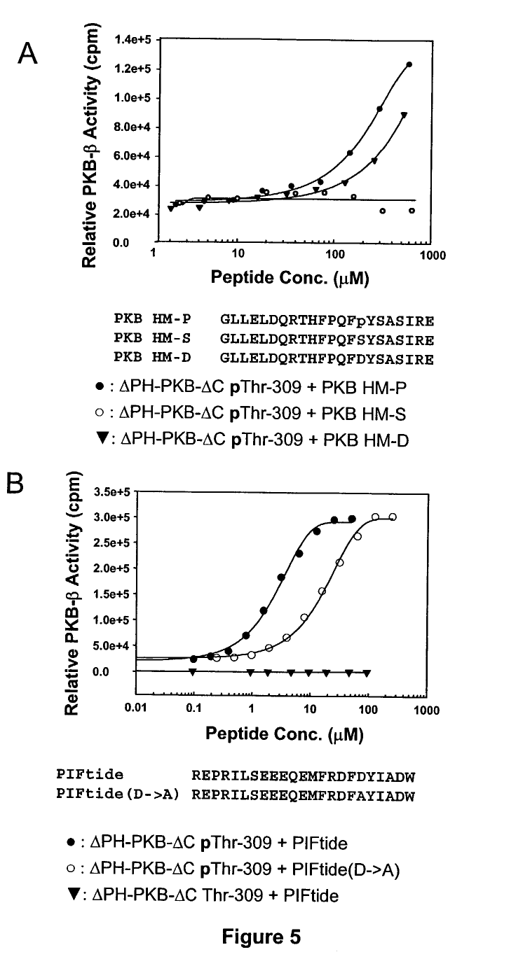

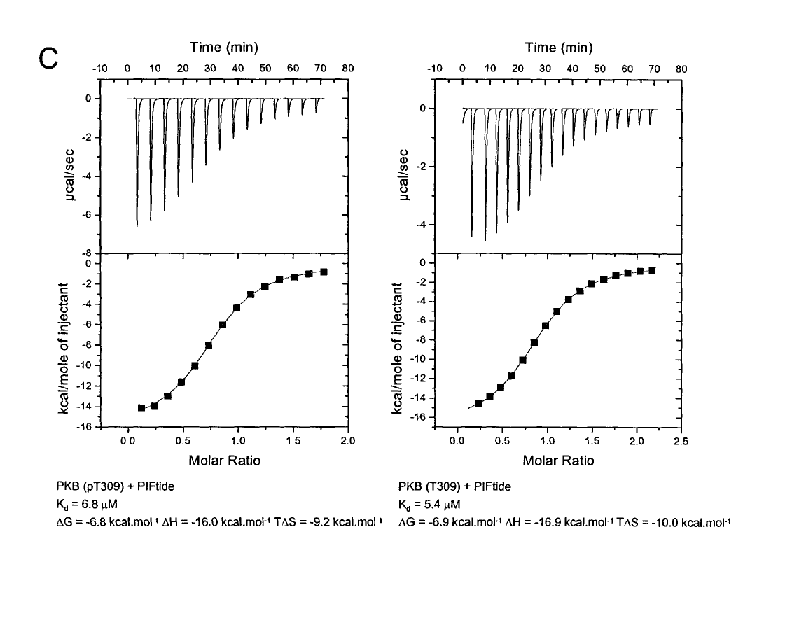

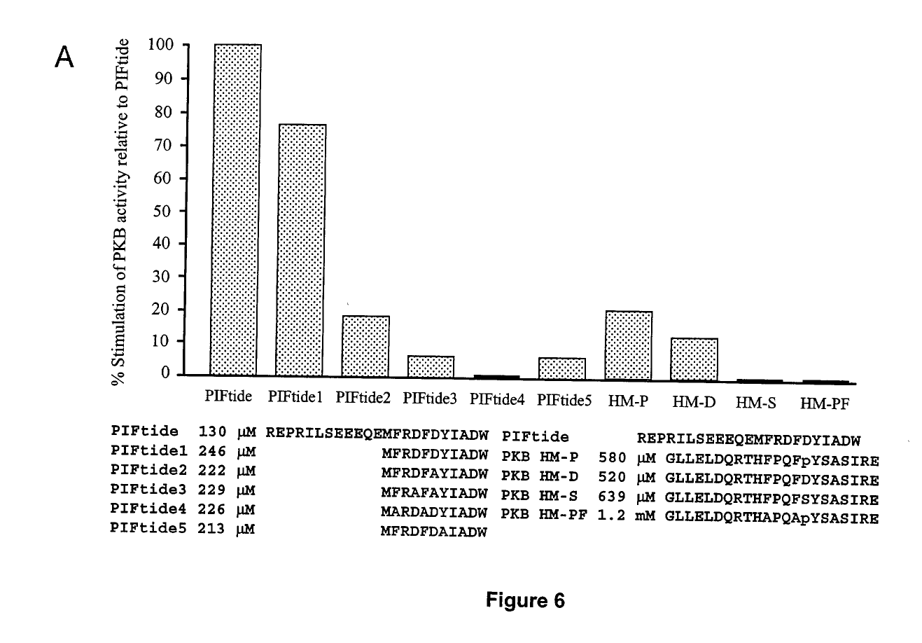

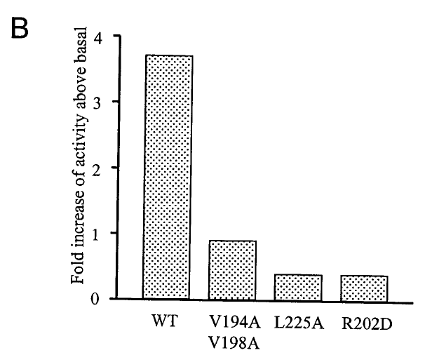

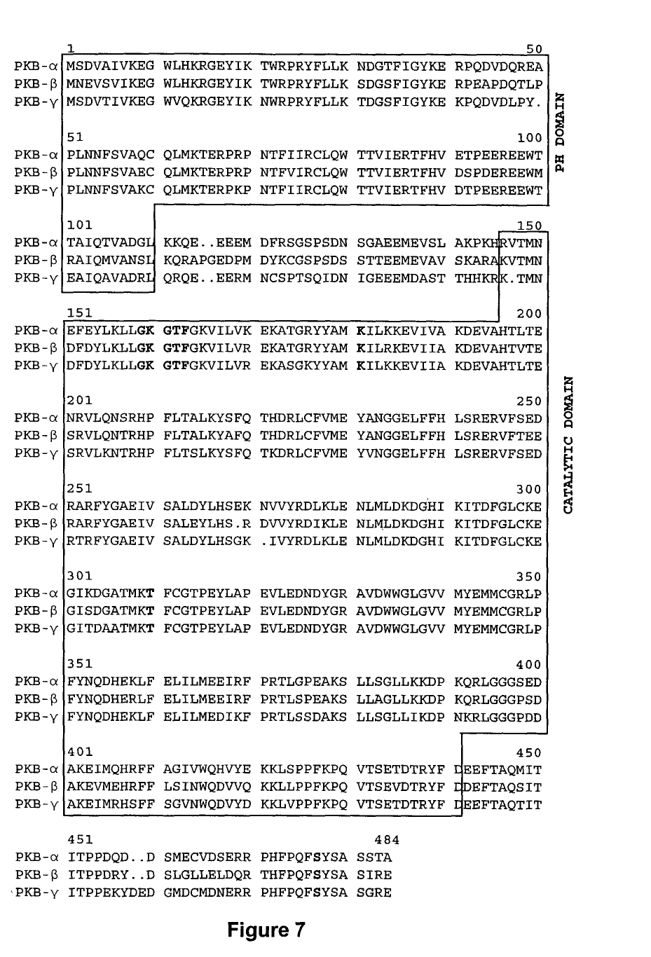

Within the N-terminal lobe of PKB, the β-sheet is well ordered, however, residues Ala-189 to Thr-207, equivalent to the αB-helix and the majority of the αC-helix of PKA, are highly mobile, as judged by disorder in the weighted 2Fo-Fc electron density, and composite simulated annealing omit maps, and analysis of the atomic temperature-factors (FIG. 2A). Specifically, for all crystal forms, there is no visible electron density to account for residues Ala-189 to Thr-197, whose counterparts in PKA form the C-terminus and N-terminus of the αB- and αC-helices, respectively.

Electron density corresponding to the main-chain of the remaining residues of the αC-helix is fragmented, and the side-chains of these residues are disordered. The short αB-helix, which connects the αC-helix with the central β3-strand of the β-sheet, is unique to the AGC-protein kinases, and causes the N-terminus of the αC-helix to be displaced from the β4/β5-strands of the β-sheet (FIG. 1, 2). As a consequence, the αC-helix packs less tightly against the hydrophobic side-chains of the β-sheet, compared with other protein kinases, and, significantly a deep surface groove is created at the interface between the αB/αC-helices and β-sheet. In PKA this groove permits interactions between the N-terminal lobe and C-terminal hydrophobic motif.

The importance of the conserved αC-helix for both catalytic and regulatory functions has been demonstrated for many protein kinases. An invariant glutamate residue located near the N-terminus of the helix, (Glu-91 of PKA, Glu-200 of PKB), is responsible for its catalytic function by forming a hydrogen bond with an invariant lysine side-chain; Lys-72 of PKA (FIG. 3). Lys-72 in turn coordinates the β-phosphate of ATP in active protein kinases.

In addition, because the αC-helix is responsible for the major interfacial contacts between the N- and C-lobes, particularly via its interactions with the DFG motif of the activation segment, it plays a role both in aligning catalytic and substrate-peptide binding residues of the C-terminal lobe, and in governing the overall juxtaposition of the N- and C-lobes.

Motion of the αC-helix represents a general mechanism for the modulation of kinase catalytic activity, and the integration of diverse regulatory signals. For example, the position of the αC-helix of CDK2 is shifted to an active conformation on the association of the monomeric CDK2 subunit with cyclin A (Jeffrey et al., 1995), and similar changes in the αC-helix are observed on activation of the insulin receptor kinase and ERK2 on phosphorylation of their activation segments (Hubbard 1997; Canagarajah et al., 1997), and in the Src- and Eph-family tyrosine kinases (Sicheri et al., 1997, Xu et al., 1997; Wybenga-Groot et al., 2001). Significantly, in many protein kinases that are regulated by phosphorylation of the activation segment, the αC-helix provides a basic residue to contact the phosphate group of the phospho-amino acid, hence coordinating the relative positions of the αC-helix with the activation segment, and the N- and C-terminal lobes. In PKA, the basic residue is His-87 at the N-terminus of the αC-helix, which contacts pThr-197 of the activation segment (FIG. 2, 3). In the inactive state of PKB, His-196 and Glu-200 of the αC-helix (equivalent to His-87 and Glu-91 of PKA) are disordered, and contacts between Glu-200 and Lys-181 (Lys-72 of PKA), and those between His-196 and pThr-309, are not formed (FIG. 3).

Disorder of the αC-helix contributes to an inactive state of PKB for two reasons. First, the side-chain of Lys-181 is not properly positioned, and second, there are associated changes in the structure of the activation segment, and relative disposition of the N- and C-terminal lobes. As described below, disorder of the αB- and αC-helices of PKB is coupled to the disorder of its non-phosphorylated C-terminal regulatory segment.

Role of the C-terminal Regulatory Segment

A distinctive structural feature of PKA, not usually observed in other protein kinases, is the interaction of the extreme C-terminus of the protein with its N-terminal lobe. In PKA, the polypeptide chain emerges from the C-terminal lobe and extends along the entire length of the bi-lobal structure. At the tip of the N-lobe, the chain forms a reverse turn, allowing the extreme C-terminal eight residues of PKA to lie within an amphipathic/hydrophobic groove on the surface of the N-lobe (FIG. 1A, 2B). Importantly, these interactions are mediated by residues of the C-terminal hydrophobic motif, which contact the surface groove formed by residues of the αB- and αC-helices, and the β5-strand of the N-lobe. The dominant interactions at the interface involve those between the side chains of the two phenylalanine residues of the hydrophobic motif, Phe-347 and Phe-350, which protrude into a pocket formed by hydrophobic residues of the N-lobe (FIG. 2). Specifically, the phenyl-ring of Phe-347 is extensively buried by the side-chains of five amino acids: Lys-76, Val-79 and Val-80 of the αB-helix, Ile-85 of the αC-helix, and Leu-116 of the β5-strand, whereas the side-chain of Phe-350 contacts Leu-89 and Lys-92 of the αC-helix, and Leu-116 and Met-118 of the β5-strand (FIG. 2B). At one end of the channel, two adjacent basic residues of the αC-helix form salt-bridge interactions with two carboxylate groups of the hydrophobic motif.

In all three crystal structures of PKB, residues corresponding to the regions of the αB- and αC-helices of PKA that interact with the hydrophobic motif, are disordered, and this probably results from loss of interactions with the hydrophobic motif of PKB (FIG. 2A, C). In the crystal structures of ΔPH-PKB-ΔC, the 21 residues C-terminal to Ser-460 were removed from the expression construct, and therefore potential interactions between the hydrophobic motif and the N-lobe are not possible. Moreover, in these structures, electron density for residues C-terminal to Asp-441 is not visible, suggesting that they are conformational disordered. However, in the ΔPH-PKB structure, which contains a non-phosphorylated hydrophobic motif, and therefore retains the potential to interact with the N-lobe, we are also unable to detect visible electron density for residues C-terminal to Asp-441, indicating that the C-terminal 40 residues, including the hydrophobic motif, are mobile.

Conformation of the Activation Segment and Nucleotide Binding Site

The activation segment is central to the regulation and catalytic activity of protein kinases (Johnson et al., 1996). In the structure of active protein kinases, the activation segment contributes to the correct conformation of the catalytic site and ATP-binding residues, and participates in peptide-substrate recognition and specificity. By functioning as a link between the N- and C-lobes, conformational changes of the activation segment, resulting from regulatory phosphorylation, and/or modulator subunits, are coupled to global changes in kinase structure. In all three crystal forms of PKB, a contiguous region of the activation segment (residues 297 to 312) located between the invariant DFG and APE motifs, and including (p)Thr-309, is disordered. There is no electron density visible for these residues in either the 2Fo-Fc, or the simulated annealling omit maps. It was determined that pΔPH-PKB-ΔC was phosphorylated on Thr-309 by quantitative mass spectroscopic analysis of a tryptic digest of the protein. Moreover, it was confirmed that the protein forming the pΔPH-PKB-ΔC crystal was phosphorylated by Western blot analysis of a dissolved crystal using pThr-309-specific antibodies.

In the inactive PKB structures, residues of the DFG sequence are ordered, but adopt a different conformation from their counterparts in PKA, functioning to inhibit PKB by disrupting the nucleotide-binding site (FIG. 3). The DFG motif of activated protein kinases is important because its Asp residue (Asp-184 of PKA) coordinates the Mg²⁺ ion responsible for contacting the β- and γ-phosphates of ATP. In PKB, the side-chain of Asp-293 (equivalent to Asp-184 of PKA) is directed away from the ATP binding site (FIG. 3B). This structural change is accompanied by a shift in the positions of Phe-294 and Gly-295 of the DFG motif, and main-chain of Leu-296, towards the glycine-rich β1-β2 nucleotide-binding loop of the N-lobe. Motion of the DFG-motif residues is accommodated by a change in the relative orientation of the N- and C-lobes of PKB, compared with PKA, to avoid their clash with the β1-strand of the N-lobe. Relative to the conformation of the equivalent Phe-185 residue of PKA, the phenyl ring of Phe-294 is displaced by as much as 10 Å, and is situated within the hydrophobic adenine-binding pocket for ATP. This structural feature of PKB is similar to the inactive state of IRK where the Phe residue of the DFG motif blocks the nucleotide-binding. site by mimicking the ATP adenine ring (Hubbard et al., 1994). Thus, in PKB, the ATP binding site is disrupted both because the Lys-181 and Asp-293, residues responsible for coordinating the phosphate groups, are displaced, and because ATP is sterically hindered from binding by Phe-294. In PKA, and in the structures of other activated protein kinases, Phe-185 of the DFG motif packs deep into the interface between the two lobes, and forms intimate contacts with hydrophobic residues of the αC-helix of the N-lobe. These interactions serve to stabilise the relative positions of the αC-helix and activation segment. The altered conformation of Phe-294 of PKB is correlated with the relative dispositions of its N- and C-lobes, and the disorder of the αC-helix.

Crystal structures of protein kinase-peptide substrate complexes indicate that a common function of the activation segment is to coordinate the peptide-substrate with the correct geometry to allow phosphorylation of the incoming hydroxyl-group of a Ser/Thr or Tyr residue (Knighton et al., 1991b, Bossemeyer, 1993, Hubbard, 1997; Lowe et al., 1997). In PKA, the P+1 region of the activation segment, immediately C-terminus to pThr-197, contributes to peptide binding. The conservation of the P+1 region amongst AGC-kinases, suggests that in the phosphorylated active state of a PKB-substrate complex, similar peptide-protein interactions will exist. Disorder of the activation segment of PKB in both the unphosphorylated and mono-phosphorylated (pThr-309) states will preclude interactions with protein substrates.

PKB Peptide Substrate Specificity

The substrate specificity of PKB is known from an analysis of physiological PKB phosphorylation sites, and from an oriented peptide library screen (Obata et al., 2000). PKB only phosphorylates peptides with an arginine at the P-3 position and also strongly prefers substrates with an Arg residue at P-5 and with large hydrophobic residues at P+1. The structural basis for this substrate specificity can be rationalised by comparing the ternary PKA complex with our structure of PKB including the activation segment modelled on that of PKA. Optimal peptide substrates of PKA are related, although not identical, to those of PKB and other AGC-kinases. In the ternary PKA structure, PKI has the sequence T-G-R-R-N-A-I-H, with Ala at P-0. Arg at P-3 forms a salt bridge to Glu-127 (Knighton et al., 1991b; Bossemeyer et al., 1993), and because this residue is also conserved in PKB and phosphorylase kinase (where it contacts an Arg at P-3, Lowe et al., 1997), it is likely that the equivalent interaction will be formed in PKB-peptide complexes. Interestingly, the side-chain of Tyr-330 of PKA that is directed towards the Arg P-3 residue is a glutamate in PKB, possibly enhancing the affinity for a peptide with an Arg at P-3. Unlike PKB, PKA does not have a preference for an Arg at P-5, and in the PKA structure, Arg-133 is in close proximity to the Thr side-chain at P-5 of PKI. In PKB, however, Arg-133 is replaced with a serine, and this less bulky residue would accommodate a potential interaction between the peptide Arg residue at P-5 and Glu-342 of PKB. Finally, PKB prefers bulky hydrophobic residues at P+1, in contrast to PKA which is only able to accommodate smaller aliphatic residues. This P+1 hydrophobic site is larger in PKB because the side-chain of Phe-359 lacks the hydroxyl group of the equivalent Tyr-247 residue of PKA.

Mechanism of PKB Activation by Phosphorylation

The crystal structures of PKB, combined with an analysis of the structural differences between PKB and an activated conformation of PKA, provides a framework for understanding the mechanism of activation of PKB by phosphorylation of Thr-309 and Ser-474. Central to the conversion to the activated state on phosphorylation, are concerted disorder to order transitions of the αB- and αC-helices, activation segment, and C-terminal regulatory segment, all of which are linked to conformational changes of the DFG motif and re orientation of the N- and C-lobes to relieve steric hindrance to ATP binding, and to align catalytic site residues. Because the known structures of activated protein kinases all share the same overall features, including juxtaposition of catalytic site, and ATP and peptide binding residues, we can assume that phosphorylation of PKB converts the enzyme into a conformation similar to that of PKA phosphorylated on Thr-197. However, what distinguishes PKB from PKA, is the requirement for phosphorylation of both the C-terminal regulatory segment and the activation segment, to activate the kinase maximally. The role of Thr-309 phosphorylation will be similar to activation segment phosphorylation of PKA, CDK2 and ERK2, namely to coordinate contacts between the activation segment and other structural elements of the protein kinase, specifically, (i) the αC-helix of the N-lobe, (ii) a conserved arginine residue immediately preceding the catalytic Asp residue (Arg-165 and Asp-166, respectively of PKA), and (iii) a basic residue of the activation segment situated close to the Asp of the DFG motif (Lys-189 of PKA). Conservation of the three basic residues of PKA that contact the phosphate group of pThr-197 in all PKB-isoforms, suggests that the equivalent charge neutralisation observed in PKA will occur in the active state of PKB. In all three crystal forms of PKB, which represent low and partially active forms of the enzyme, and includes pΔPH-PKB-ΔC phosphorylated on Thr-309, the activation segment is disordered, and the enzyme adopts an inactive conformation. Thus phosphorylation of Thr-309 alone is not sufficient to order the activation segment and promote an active state of the enzyme; additional phosphorylation of Ser-474 is required. The hydrophobic motif of PKA is not regulated by phosphorylation, and in the PKA crystal structure lies within a surface hydrophobic groove formed by residues whose counterparts in the αB- and αC-helices of the inactive states of PKB are disordered. The finding that the C-terminal regulatory segment, comprising the unphosphorylated hydrophobic motif of ΔPH-PKB was disordered, suggests that activation by Ser-474 phosphorylation is caused by the concomitant ordering of the regulatory segment and αB- and αC-helices mediated by the interaction of the motif with the induced N-terminal lobe surface groove. Ordering of the αC-helix will induce global changes in the PKB conformation by facilitating interactions between the residues of the αC-helix and critical regions of the molecule. These interactions include those between Lys-181 and Glu-200, and two αC-helix-activation segment interactions; His-196 and pThr-309, and hydrophobic contacts with Phe-294 of the DFG motif. Reconfiguration of the activation segment allows the correct alignment of catalytic site and substrate binding residues. Consistent with this model of activation by ordering of the regulatory segment induced by Ser-474 phosphorylation, previous studies of PKA suggested that an ordered hydrophobic motif is important for enzyme activity and stability.

Replacing the conserved Phe residues of the motif with alanines, reduces catalytic activity to only 0.5% of the wild-type enzyme, and leads to decreased thermal stability (Etchebehere et al., 1997).

Allosteric Activation of pThr 309-PKB by Hydrophobic Motif Peptides

To test the model that Ser 474 phosphorylation promotes an interaction between the hydrophobic motif and the induced hydrophobic groove of the N-terminal lobe, thereby causing an allosteric activation of the kinase, the ability of peptides modelled on the hydrophobic motif of PKB to activate the enzyme via an intermolecular association with the N-terminal lobe was assessed.

First, it was shown that towards Crosstide, a peptide-substrate derived from the PKB phosphorylation site of GSK-3, the unphosphorylated form of ΔPH-PKB-ΔC has no significant catalytic activity, whereas its Thr 309 phosphorylated counterpart was active. Addition of a peptide modelled on the phosphorylated hydrophobic motif of PKBβ (HM-P, residues 460-481), activated pΔPH-PKB-ΔC, with the stimulation reaching a maximum of 4-fold at 0.6 mM, the highest concentration of HM-P peptide achievable in our assay (FIG. 5A). Significantly, this 4-fold stimulation of PKB by HM-P peptide is lower than the 7-10 fold stimulation of PKB by Ser 474 phosphorylation (Alessi et al., 1996). Analysis of the concentration-dependent activation of PKB by HM-P (FIG. 5A) revealed that the binding sites for HM-P on ΔPH-PKB-ΔC were not fully titrated even at a peptide concentration of 0.6 mM, suggesting that higher concentrations of HM-P are necessary to fully stimulate PKB activity. The modest activation of PKB by HM-P peptide suggests a relatively low affinity of peptide for the PKB N-terminal lobe. An equivalent HM-peptide with an Asp substitution of Ser 474 was also capable of activating pΔPH-PKB-ΔC, consistent with studies showing that Asp mimics Ser 474 phosphorylation (Alessi et al., 1996). However, the maximum activation by this peptide was only 3-fold because of the lower affinity towards ΔPH-PKB-ΔC than the HM-P peptide (FIG. 5A). Finally, as expected, the unphosphorylated HM-peptide did not stimulate PKB activity. It was also found that the phosphorylated HM-peptide did not further activate ΔPH-PKB phosphorylated on both Thr 309 and Ser 474. Furthermore, HM-P peptide was unable to activate ΔPH-PKB-ΔC with unphosphorylated Thr 309, in agreement with earlier findings that growth factor stimulation fails to activate T308A mutants of PKBα (Bellacosa et al., 1998) indicating an essential role of Thr 308/309 phosphorylation for PKB activity.

Phosphorylation of a Ser or Thr residue within the hydrophobic motif is a conserved feature of the activation of varied AGC-kinases, including PKC (Keranen et al., 1995) and the p70 and p90 S6-kinases (Pearson et al., 1995; Frodin et al., 2000). However, in some PKC isoforms, and in the PKC related kinase, PRK2, the site of Ser/Thr phosphorylation is replaced with either an Asp or Glu residue, suggesting that in these kinases, the hydrophobic motif will be constitutively activated, similarly to PKA, because of a permanent negative charge at this site. The C-terminal region of PRK2 that encompasses the carboxy-terminal hydrophobic motif was previously shown by Alessi and colleagues to interact tightly with the AGC-family kinase PDK1 (Balendran et al., 1999). PIFtide, a peptide representing the C-terminal 24 residues of PRK2, including its hydrophobic motif, was observed to stimulate PDK1 activity by four-fold (Biondi et al., 2000). Remarkably, PIFtide was found here to activate pΔPH-PKB-ΔC by 15-fold, substantially more strongly than the activation achieved by the phosphorylated HM-peptide. Analysis of the concentration dependence of pΔPH-PKB-ΔC activation by PIFtide, revealed that the peptide binds the kinase with high affinity, resulting in a maximum and saturable activation at 20 μM and a corresponding EC₅₀ value of 3 μM (FIG. 5B). Significantly, the specific activity of pΔPH-PKB-ΔC maximally activated by PIFtide was 350 nmol/min/mg, essentially identical to the specific activity of ΔPH-PKB phosphorylated on both Thr 309 and Ser 474. These specific activity data indicate that the stimulation of pΔPH-PKB-ΔC by an intermolecular association with PIFtide is equivalent to Ser 474 phosphorylation and the resultant intramolecular association between the N-lobe of PKB and phosphorylated HM and furthermore suggests that an analysis of PKB-PIFtide interactions will provide insights concerning the mechanism of activation by Ser 474 phosphorylation. PIFtide promotes a 5-fold activation of ΔPH-PKB phosphorylated on Thr 309 to a specific activity similar to that of pΔPH-PKB-ΔC. The lower level of stimulation relative to the 15-fold observed for pΔPH-PKB-ΔC can be explained by the partial phosphorylation of Ser-474 on pΔPH-PKB purified from Sf9 cells.

Using isothermal titration calorimetry, the affinity between PIFtide and both pΔPH-PKB-ΔC and ΔPH-PKB-ΔC was determined (FIG. 5C). Firstly, we found that the equilibrium dissociation constant defining the interaction between PIFtide and pΔPH-PKB-ΔC was 6 μM, essentially identical to the EC₅₀ value for the activation of pΔPH-PKB-ΔC by PIFtide (FIG. 5B). This result suggests that the association of PIFtide to PKB correlates with the activation of the kinase. Secondly, it was found that the interaction of PIFtide with ΔPH-PKB-ΔC is driven by a large negative enthalpy change (ΔH of −16.0 kcal.mol⁻¹) that compensates the energetically unfavourable decrease in entropy (TΔS of −9.2 kcal.mol⁻¹). The observed large decrease in entropy is not generally typical of protein-peptide interactions, for example SH2-domain-phosphotyrosine peptide complexes (Ladbury et al., 1996), and is consistent with an ordering of both the protein, presumably the αB- and αC-helices of the N-lobe, and peptide on complex formation. Although PIFtide does not stimulate the activity of ΔPH-PKB-ΔC (FIG. 5B), ITC data revealing a dissociation constant of 5.5 μM indicated that PIFtide interacts with this form of the enzyme as strongly as it does to phosphorylated ΔPH-PKB-ΔC, further emphasising the crucial role of Thr 309 phosphorylation for PKB activity (FIG. 5C).

The finding that PIFtide interacts with PKB with high affinity provided a model system for testing the notion that the essential role of Ser 474 phosphorylation is to promote the association of the hydrophobic motif with the N-lobe of PKB. The residue of PIFtide equivalent to Ser 474 of PKB is an Asp, which presumably mimics a phosphorylated Ser 474 residue. To assess the importance of this residue for the ability of PIFtide to activate PKB, the concentration dependent activation of pΔPH-PKB-ΔC by PIFtide with an Ala residue substituting for the Asp was determined. Although higher concentrations of this mutant PIFtide(D->A) are required to activate pΔPH-PKB-ΔC than wild type PIFtide, suggesting a lower affinity, the maximal activation of the kinase achieved by saturating concentrations of the mutant peptide is identical to that of the wild type peptide (FIG. 5B). The estimated EC₅₀ value for PIFtide(D->A) is 30 μM, indicating a 10-fold lower affinity than PIFtide. ITC experiments also revealed an approximately 25-fold lower affinity between PIFtide(D->A) and pΔPH-PKB-ΔC relative to PIFtide. Thus, these experiments demonstrate an important concept that the PIFtide-induced conformational change of pΔPH-PKB-ΔC that results when PIFtide interacts with the kinase, and which leads to a maximal stimulation of the kinase activity, does not require a negatively charged residue at the equivalent of Ser 474 of the hydrophobic motif. The major role of a negative charge at this site is to increase the association of PIFtide with the PKB N-lobe, and that other residues, particularly the conserved Phe residues of the FxxF motif (see below), are more critical for promoting the conformational change of the protein.

Because of the low affinity between pΔPH-PKB-ΔC and the PKB HM peptides, it was not possible to determine a K_{D} value defining their interaction with PKB using ITC. However, by assuming that the association between pΔPH-PKB-ΔC and the PKB HM-peptides is an equilibrium process and that at saturating concentrations of peptide, the activation of pΔPH-PKB-ΔC will be similar to that induced by PIFtide, the data in FIG. 5A were used to estimate the EC₅₀ constants for the phosphorylated and S474D HM-peptides to be 2.3 mM and 3.6 mM, respectively, an affinity ˜1000-fold lower than for PIFtide.

Mutagenesis of the Hydrophobic Motif and N-lobe Hydrophobic Groove

By assessing the ability of modified PIFtide and HM peptides to activate pΔPH-PKB-ΔC, the role of conserved residues of the hydrophobic motif (HM) to induce the active conformation of PKB was delineated. These experiments used an 11-residue peptide encompassing the six-residue hydrophobic motif of PIFtide (PIFtide1, .FIG. 6A) that essentially recapitulates the activation of pΔPH-PKB-ΔC observed for the 24-residue PIFtide. The slightly lower activation suggests that residues of PIFtide N-terminal to the HM contribute to high affinity PKB interactions. The PKB activities were determined at PIFtide concentrations ranging from 210-250 μM, where wild-type PIFtide fully activates PKB (FIGS. 5B, 6A). While all conserved residues of the HM motif contribute to PKB activation, significantly, the two phenylalanine residues of the motif are essential for HM-induced activation. Ala substitutions of these residues in both PIFtide and the phosphorylated PKB HM-peptide, completely eliminated the potential of these peptide to stimulate PKB, even at PKB HM-peptide concentrations of 1.2 mM (FIG. 6A). A similar essential role for the equivalent Phe residues has been proposed for PKA where Ala substitutions lower the thermal stability, and virtually abolishes the catalytic activity of the enzyme (Etchebehere et al., 1997). Mutation of either the conserved Tyr residue or of both Asp residues of the PIFtide motif showed that these residues also contribute to the stimulatory affect of PIFtide on PKB activity (FIG. 6A). PIFtide activates PKB by interacting with, and simultaneously stabilising the activated conformation of PKB. Therefore, the lower stimulatory effect of mutant PIFtide and PKB peptides most likely results from a reduced affinity for the activated conformation of PKB, however, because mutant PIFtide peptides have either low or no activity even at >200 μM, we were unable to determine EC₅₀ values for their activation of PKB.

The crucial role of the conserved Phe residues of the hydrophobic motif to promote PIFtide and PKB HM-peptide mediated stimulation of PKB, and for the activity of PKA, suggests that they stabilise the active state of both PKB and PKA by a related structural mechanism. To test the notion that a hydrophobic groove is induced in PKB to engage the hydrophobic motif, and activate the kinase, a series of His tagged pΔPH-PKB-ΔC hydrophobic groove mutants were prepared and their responsiveness to PIFtide assessed. PKB mutants were transiently expressed in HEK cells, phosphorylated in vitro with PDK1, and purified using Ni-NTA agarose. SDS-PAGE and western blot analysis of the purified fractions revealed that wild type and mutant proteins were expressed to similar levels, and that the enzyme was quantitatively isolated in a phosphorylated state. Moreover, the basal kinase activities of wild type and mutant proteins were similar, indicating that the mutations did not disrupt the overall structure of the protein. Wild type PKB prepared using this procedure was stimulated ˜5-fold by 130 μM PIFtide (FIG. 6B). The slightly lower activation probably results from incomplete Thr 309 phosphorylation, and consequently the PKB HM-peptide did not elicit measurable activation. The substitution of hydrophobic groove residues significantly reduced, but did not completely abolish the potential of PIFtide to stimulate PKB (FIG. 6B). Mutation of two αC-helix residues, Val 194 and Val 198 (Ile 85 and Leu 89 of PKA), reduced PIFtide activation to only 25% of wild type, whereas a Leu 225 mutant of the β-5 strand (Leu 116 of PKA) caused almost a complete loss of responsiveness to PIFtide (FIGS. 2, 4, 6B).

Electrostatic interactions are important in defining high affinity PIFtide and PKB HM peptide associations with PKB (FIG. 5B), and form the basis for the increased affinity of the HM for the N-lobe and subsequent activation of PKB by Ser 474 phosphorylation. Examination of the PKA and PKB crystal structures suggests that Arg 202 of the αC-helix is likely to be important in mediating contacts to pSer 474 and the corresponding Asp residue of PIFtide. The equivalent residue of PKA, Arg 93, which is also conserved in PKC and PRK2, forms a water-mediated salt bridge to the carboxylate group of Glu 349 (FIG. 2). A charge reversal at this site (R202D) almost eliminates the ability of 130 μM PIFtide to activate PKB (FIG. 6B), consistent with the notion that Arg 202 forms electrostatic contacts with PIFtide. However, analogous to the finding that at high concentrations, the PIFtide(D->A) mutant could activate PKB maximally (FIG. 5B), the R202D PKB mutant was more responsive to higher concentrations of the peptide.

Conservation of the Hydrophobic Motif Groove in AGC-Protein Kinases

The role of a phosphorylated hydrophobic motif to activate PKB that is described here, is probably applicable to other AGC-protein kinases that are regulated via dual phosphorylation of an activation segment residue and a hydrophobic motif residue, for example PKC, the p70 and p90 S6 kinases and SGK (Parekh et al., 2000; Pearson et al., 1995; Frodin et al., 2000; Kobayashi and Cohen, 1999). A disorder-order transition of PKC induced by phosphorylation is implied by the resistance of the fully phosphorylated, but not partially phosphorylated forms of PKC, to protein phosphatases, and their enhanced resistance to temperature-induced denaturation (Bornancin and Parker, 1997). Substitutions of the Phe residues of the hydrophobic motif of PKA lowers its thermal stability, and virtually abolishes its catalytic activity (Etchebehere et al., 1997). The conservation of the hydrophobic motif of AGC-kinases is correlated with the invariance of the residues equivalent to Lys-76 and Leu-116 of PKA that would be predicted to form the base of the hydrophobic groove in a number of diverse AGC-kinases, including PKA, PKB, PKC, p70-S6K, p90-S6K, SGK, NDR and PDK1. Alessi and colleagues have shown that the hydrophobic motif of PIFtide determines the ability of this peptide to bind to the N-lobe, hence activating PDK1 (Balendran et al., 1999), and the presence of PIFtide greatly increases the thermal stability of PDK1 (Biondi et al., 2000). By analogy to PKB, we suggest that the activation of PDK1 by PIFtide involves a disorder-order transition of the αB- and αC-helices, and consequent global conformational changes.

Structure-Based Drug Design

Determination of the 3D structure of PKBβ provides important information about the binding sites of PKBβ, particularly when comparisons were made with similar enzymes. This information may then be used for rational design of PKBβ inhibitors, e.g. by computational techniques which identify possible binding ligands for the binding sites, by enabling linked-fragment approaches to drug design, and by enabling the identification and location of bound ligands using X-ray crystallographic analysis.

Since all known small molecule inhibitors of protein kinases are competitive with ATP, and therefore interact with the ATP binding site, an understanding of the PKB residues involved in the interaction with ATP enables the development of specific and potent inhibitors of this kinase. This information may thus be used to develop potent and specific small molecule inhibitors of PKB in a number of ways. PKBβ may be co-crystallised, and/or existing PKBβ crystals may be soaked, with known inhibitors of PKB, including staurosporin, and those discovered in high-throughput screening programmes known to the skilled person. Alternatively, or additionally, rational drug design programmes may make full use of the crystallographic coordinates.

Discussion and Implications for Other AGC Kinases

This study presents a model for the regulation of PKB by hydrophobic motif phosphorylation. The data indicates that the role of HM phosphorylation is to induce an ordered N-terminal lobe as a result of an increased affinity between the hydrophobic motif and the hydrophobic groove. Ordering of the αC-helix transmits a structural change to the activation segment and re-orients the N- and C-lobes. In the inactive PKB crystal structures residues of the αB- and αC-helices are disordered. Consistent with a disorder-to-order transition, the interaction of PIFtide with PKB is accompanied by a large negative entropy change. Mutation of key hydrophobic residues of the N-lobe groove and hydrophobic motif either reduce or eliminate the ability of PIFtide to activate PKB. Using PIFtide as a model system shows that the role of a negative charge within the HM (e.g. PKB Ser 474 phosphorylation) is to increase the affinity of the HM for the N-lobe.

In the context of the PKB kinase domain, phosphorylation of Ser 474 will increase the ability of the HM to interact with the N-lobe via an intramolecular association. However, because PIFtide(D->A) had only 10-fold lower affinity for PKB relative to PIFtide (FIG. 5B), it is likely that the unphosphorylated HM of PKB will still retain a weak affinity for the N-lobe. It can therefore be rationalised why PKB mono-phosphorylated on Thr 309 has between 7-10-fold lower activity than doubly phosphorylated PKB.

Disorder to order transitions of the αC-helix as a result of phosphorylation represents a previously unrecognised mechanism for the stimulation of protein kinase activity. However, there is evidence that other AGC-kinases undergo similar transitions, modulated by the hydrophobic motif. For example, phosphorylation of the HM of PKC increases its resistance to temperature-induced denaturation (Bornancin and Parker, 1997) and the Phe residues of the PKA HM motif are critical for its stability and catalytic activity (Etchebehere et al., 1997). The conservation of the hydrophobic motif of AGC-kinases is correlated with the invariance of the residues equivalent to Lys 76 and Leu 116 of PKA predicted to form the base of the hydrophobic groove in a number of diverse AGC-kinases, (FIG. 4). Uniquely amongst AGC-kinases, PDK1 lacks a C-terminal hydrophobic motif, although its N-terminal lobe hydrophobic groove is proposed to interact with PIFtide (Biondi et al., 2000). Similarly to the findings with PKB, high affinity interactions between PIFtide and PDK1 required the conserved aromatic and Asp residues of the hydrophobic motif of the peptide (Balendran et al., 1999), and were disrupted by substitutions of PDK1 HM groove residues (Biondi et al., 2000).

The affinity of the HM-P peptide for PKB that is not phosphorylated on Ser 474 is ˜1000-fold lower than that of PIFtide, and is reminiscent of the low affinity of the tyrosine phosphorylated C-terminus of Src for its own SH2 domain, compared with optimal phosphotyrosine binding sequences (Bradshaw et al., 1998). The covalent attachment of the phosphorylated hydrophobic motif to the PKB kinase domain will greatly increase its effective concentrations presumably in excess of the EC₅₀ value estimated for the activation of PKB by the HM-P peptide. However, a modest mutual affinity may be important for two reasons. First, in order for phosphorylation of the HM to be capable of modulating its affinity for the N-lobe, the affinity of the unphosphorylated HM for the N-lobe must be sufficiently low that it is not constitutively associated with the N-lobe. For example, a substitution of PIFtide(D->A) for the PKB HM motif would render PKB fully active and therefore unresponsive to HM phosphorylation. Second, it allows modulator proteins to gain access either to the hydrophobic groove or the phosphorylated motif, or for protein phosphatases to dephosphorylate pSer 474. Whether the activation of PKB by PIFtide reflects a biologically significant regulatory mechanism for stimulation of PKB by a modulator protein that interacts with the N-lobe is unknown. However, the affinity of PIFtide for PKB may provide insight concerning the nature of the PDK2 enzyme responsible for phosphorylating Ser 474. A possible candidate for this enzyme is a kinase that interacts with the hydrophobic binding groove of PKB, perhaps via a sequence similar to the hydrophobic motif of PKB or PIFtide.

### MATERIALS AND METHODS

The Genebank accession numbers for the PKB isoforms are as follows:

α gi 190827 (m63167); β gi 178325 ( m95936); γgi 4757578 (af124141)

Expression of ΔPH-PKBβ-ΔC (Residues 146-460) and ΔPH-PKBβ (Residues 146-481)

Generation of recombinant baculovirus using the GIBCO/Life Sciences Bacmid system was performed using standard procedures.

For ΔPH-PKBβ-ΔC, 3 PCR reactions were set up as follows:

| | | | |
|---|---|---|---|
| 36.5 | μl | H₂O | |
| 5 | μl | 10x pfu buffer | |
| 5 | μl | dNTPs | 0.2 mM |
| 1 | μl | 5′ Primer 36117 | 60 pmols |
| 1 | μl | 3′ Primer 36508 | 60 pmols |
| 1 | μl | PfastBacHTa ΔPH PKBβ2851a | (170 ng) |
| 50 | μl | total + 2.5 u pfu | |

Pfu polymerase and buffer were purchased from Promega (M7741). All PCR reactions were performed in a Perkin Elmer Geneamp PCR system 9700.

| | |
|---|---|
| PCR conditions | 60 s at 95° C., |
| then 15 cycles: | 60 s at 95° C. |
| | 120 s at annealing temperature 62° C. |
| | 180 s at 72° C. |

Primers were:

| | |
|---|---|
| 36117: | GCC ATG GAT CCG AAA GTG ACC ATG AAT GAC TTC |
| | (5′ BamHI) |
| 36508: | GGG GGT ACC TCA GAG GCT GTC ATA GCG GTC AGG |
| | (3′ KpnI) |

For ΔPH-PKBβ, 3 PCR reactions were set up as follows:

| | | |
|---|---|---|
| 37 μl | H₂O | |
| 5 μl | 10x pfu buffer | |
| 5 μl | dNTPs | 2.5 mM |
| 1 μl | 5′ Primer 36117 | 70 pmols |
| 1 μl | 3′ Primer 28585 | 54 pmols |
| 1 μl | PFaStBaCHTa.ΔPH PKBβ 2702b | (200 ng) |
| 50 μl | total + 2.5 u pfu | |

Pfu polymerase and buffer were purchased from Promega (M7741). All PCR reactions were performed in a Perkin Elmer Geneamp PCR system 9700

| | |
|---|---|
| PCR conditions | 60 s at 95° C., |
| then 15 cycles: | 60 s at 95° C. |
| | 120 s at annealing temperature 66° C. |
| | 180 s at 72° C. |

Primers were:

36117:
GCC ATG GAT CCG AAA GTG ACC ATG AAT GAC TTC
(5′ BamHI)
28585:
GGG GGT ACC TCA CTC GCG CAT GCT GGC CGA GTA GG
(3′ KpnI)

All PCR fragments were pooled and purified using the Qiagen PCR purification kit (28106) and digested with the appropriate restriction enzymes and subcloned into the pFastBacHTa (10584-027) vector from Gibco BRL life technologies.

Ligation mixes were used to transform E. coli XL1 blue (Stratagene) and colonies containing recombinant DNA were grown up for miniprep DNA analysis. Miniprep was prepared using Qiagen miniprep kit 27106. All expression constructs were fully sequenced on an Applied Biosystems 3700 automated sequencer.

Insect cells (density ˜2.0×10⁶ cells/ml, total volume of 2.7 L; 5.4×10⁹ Sf9 cells), grown in a culture of GIBCO/Life Sciences supplemented Sf900II medium were infected at a moiety of infection of 2 and grown for 72 hours prior to harvesting.

Purification of ΔPH-PKBβ-ΔC (Residues 146-460) and ΔPH-PKBβ (Residues 146-481)

All procedures were performed at 4° C.

1. Cell lysis: Insect cells were lysed in a Q-sepharose buffer A (25 mM Tris.HCl, [pH 7.5], 25 mM NaCl, 25 mM NaF, 25 mM β-glycerophosphate, 0.1% (v/v) β-mercaptoethanol, 2 mM benzamidine, 0.2 mM PMSF, 10% (v/v) glycerol, 1 μg/ml of DNAase.

2. Q-sepharose, anion exchange chromatography: The lysate was cleared by centrifugation and loaded onto a 50 mL Q-sepharose column equilibrated in buffer A. The column was washed in 200 mL of buffer A and PKB was eluted using 100 mL of buffer A+1 M NaCl.

3. Ni-NTA affinity chromatography: The pH of the eluate was raised to 8.0 using a 1 M of Tris.HCl (pH 9.2) and this sample was loaded onto a Ni-NTA agarose column containing 10 mL of Ni-NTA agarose resin that had been pre-equilibrated in buffer B: 20 mM imidazole, 20 mM Tris.HCl (pH 8.0), 25 mM NaF, 25 mM β-glycerophosphate, 500 mM NaCl, 0.1% (v/v) β-mercaptoethanol, 2 mM benzamidine, 0.2 mM PMSF. The column was washed and the protein was eluted using buffer B+300 mM imidazole. EDTA and DTT to final concentrations of 0.5 mM and 2 mM, respectively, were added immediately to the eluted protein. Phosphorylation reactions (see below) were performed after this step.

4. Phenyl TSK hydrophobic interaction chromatography: The protein was brought to the appropriate concentration of ammonium sulphate and loaded onto a phenyl TSK column equilibrated in buffer C: 50 mM Tris.HCl (pH 7.5), 100 mM NaCl, 2 mM DTT, 2 mM benzamidine, 0.2 mM PMSF, with the same concentration of ammonium sulphate as the protein solution. The column was washed and PKB was eluted using a linear gradient developed to a buffer D consisting of 50 mM Tris.HCl (pH 7.5), 100 mM NaCl, 15% (v/v) glycerol, 2 mM DTT, 2 mM benzamidine, 0.2 mM PMSF.

Concentrations of ammonium sulphate used were as follows:

| | |
|---|---|
| pΔPH-PKBβ-ΔC | 1.23 M ammonium sulphate |
| ΔPH-PKBβ-ΔC | 0.63-0.68 M ammonium sulphate |
| ΔPH-PKBβ | 0.82-0.86 M ammonium sulphate |

Following this HIC step, those proteins which were to be dephosphorylated were treated with λ protein phosphatase, as described below.

5. Tev protease cleavage. The 6×His affinity tag was removed by cleavage using Tev (tobacco etch virus) protease. Tev protease was added to PKB from step 4 and this solution was dialysed over 14 hr into buffer E: 50 mM Tris.HCl (pH 8.0), 100 mM NaCl, 5 mM DTT.

6. To remove Tev protease (as well as PDK1 and λ protein phosphatase, where appropriate) from PKB after cleavage of the His-tag from PKB, the solution of Tev protease and PKB were dialysed into buffer B: 20 mM imidazole, 20 mM Tris.HCl (pH 8.0), 25 mM NaF, 25 mM β-glycerophosphate, 500 mM NaCl, 0.1% (v/v) β-mercaptoethanol, 2 mM benzamidine, 0.2 mM PMSF and loaded onto a Ni-NTA agarose column. Cleaved PKB was recovered in the flow through.

7. Q-sepharose, anion exchange chromatography. The PKB collected in step 6 was dialysed into Q-sepharose buffer F: 25 mM Tris.HCl (pH 7.5), 25 mM NaCl, 5% (v/v) glycerol, 0.5 mM EDTA, 2 mM DTT, 0.2 mM PMSF. The column was washed in the above buffer and the protein was eluted by developing a shallow gradient to buffer F+0.5 M NaCl.

8. Size exclusion chromatography. The protein from step 7 was concentrated to <2 mL and loaded onto an S75 gel filtration column equilibrated in buffer G: 10 mM Tris.HCl (pH 7.5), 100 mM NaCl, 2 mM DTT.

Expression of PDK1

Recombinant PDK1, for phosphorylation of ΔPH-PKβ-ΔC, was expressed from recombinant baculovirus generated by standard procedures.

3 PCR reactions were set up as follows using pCMV5.myc PDK1 fl-1 (Pullen et al., 1998) as a template:

| | | |
|---|---|---|
| 23 μl | H₂O | |
| 5 μl | 10x Taq buffer | |
| 10 μl | Q-solution (5x) | |
| 5 μl | dNTPs | 0.25 mM |
| 4 μl | 5′ Primer 30665 | 60 pmols |
| 1 μl | 3′ Primer 22777 | 60 pmols |
| 2 μl | pCMV5.myc PDK1 fl-1 | (200 ng) |
| 50 μl | total + 2.5 u pfu | |

Taq polymerase, Q-solution and buffer were purchased from Qiagen 201203. All PCR reactions were performed in a Perkin Elmer Geneamp PCR system 9700

| | |
|---|---|
| PCR conditions | 60 s at 94° C., |
| Then 5 cycles: | 30 s at 94° C. |
| | 4 min at 72° C. |
| then 5 cycles: | 30s at 94° C. |
| | 4 min at 70° C. |
| then 20 cycles: | 30s at 94° C. |
| | 4 min at 68° C. |

Primers

| | | |
|---|---|---|
| 30665 | CCT GCT AGC ACG GCC AGG ACC ACC AGC CAG CTG TAT GAC | NheI |
| 22777 | CCC GAA TTC TCA CTG CAC AGC GGC GTC CGG GTG GC | EcoRI |

All PCR fragments were pooled and purified using the Qiagen PCR purification kit (28106) and digested with the appropriate restriction enzymes and subcloned into the vectors indicated below. The PCR fragment was subcloned into pRSETA as a NheI/KpnI fragment, subsequently released as a NdeI/KpnI fragment and subcloned into pFastBac1 (10360-014 from Gibco BRL life technologies) between the BamHI and KpnI sites using a BamHI-NdeI linker.

All PCR fragments were pooled and purified using the Qiagen PCR purification kit (28106) and digested with the appropriate restriction enzymes and subcloned into pFastBac1 (10360-014) from Gibco BRL life technologies to yield pFastbac1. His PDK1-c(full length aa 1-556).

Ligation mixes were used to transform E. coli XL1 blue (Stratagene) and colonies containing recombinant DNA were grown up for miniprep analysis.

Miniprep was prepared using Qiagen miniprep kit 27106. All expression constructs were fully sequenced on Applied Biosystems 3700.

Insect cells (density ˜2.0×10⁶ cells/ml, total volume of 2.7 L; 5.4×10⁹ Sf9 cells), grown in a culture of GIBCO/Life Sciences supplemented Sf900II medium were infected at a moiety of infection of 2 and grown for 72 hours prior to harvesting.

Purification of PDK1

PDK1 was purified by following steps 1, 2, 3 5 and 6 described above, as for recombinant PKB.

Phosphorylation of ΔPH-PKBβ-ΔC (Residues 146-460) and ΔPH-PKBβ (Residues 146-481) on Thr309 Using PDK1

PKB from step 3 above was dialysed into a buffer containing 50 mM Tris.HCl (pH 7.5), 100 mM NaCl, 5 mM DTT. MgCl₂ and ATP were added to a final concentration of 5 mM. PDK1 was added and the mixture was incubated at 4° C. for 14 hrs and at 20° C. for 2 hrs. PDK1 was removed from phosphorylated PKB by Ni-NTA agarose. The PKB-PDK1 solution was dialysed into buffer B (step 3) and loaded onto Ni-NTA agarose and eluted as described in step 3. The phosphorylated PKB was further purified using steps 4-8 above.

Dephosphorylation of ΔPH-PKBβ (Residues 146-481) Using λ Protein Phosphatase

ΔPH-PKBβ (residues 146-481) was dialysed into the following buffer: 50 mM Tris.HCl (pH 7.5), 150 mM NaCl, 2 mM MnCl₂, 5 mM DTT, λ protein phosphatase was added at a ratio of 1 mg of λ protein phosphatase to 8 mg of ΔPH-PKBβ. ΔPH-PKBβ was incubated in these conditions at 20° C. for 2 h. Simultaneously, TEV protease was added to cleave the N-terminal His tag. After 2 h ΔPH-PKBβ was dialysed into buffer B (step 3) and loaded onto a Ni-NTA column to remove λ phosphatase and TEV. PKB was collected in the flow through. The protein was further purified using Q-sepharose and gel filtration chromatography (steps 7 and 8).

Crystallisation of pΔPH-PKBβ-ΔC (Residues 146-460)—First Batch.

The protein was concentrated to 10 mg/ml and AMPPNP/MgCl₂ was added to a final concentration of 5 mM. Crystals were grown using the under-oil batch method. A small volume of protein (3 μl) was mixed with an equal volume of crystallisation buffer: 30% (w/v) polyethylene glycol 4000, 0.2 M lithium sulphate, 0.1 M Tris.HCl (pH 7.5), 5 mM DTT, within individual wells of a 72 well polystyrene tray (Nunc) and immersed under 5 ml of silicone oil. The trays were incubated at 20° C. and crystals appeared within a few days and grew to a maximum size of 0.1 mm×0.1 mm×0.5 mm in a week. The crystals exhibited a rod-like rectangular morphology.

Crystallisation of pΔPH-PKB-ΔC (Second Batch), ΔPH-PKB-ΔC, and ΔPH-PKB.

The protein was concentrated to 10 mg/ml and AMP-PNP/MgCl₂ was added to a final concentration of 5 mM. Crystals were grown using the under-oil batch method. A small volume of protein (1 μl) was mixed with an equal volume of crystallisation buffer: 30% (w/v) polyethylene glycol 4000, 0.2 M lithium sulphate, 0.1 M Tris.HCl (pH 8.5), 5 mM DTT, within individual wells of a 72 well polystyrene tray and immersed under silicone oil and incubated at 20° C.

Data Collection and Structure Determination

Preparation of crystals for X-ray data collection: Crystals were harvested from the crystallisation trays and incubated in a cryoprotection buffer consisting of 18% (w/v) polyethylene glycol 4000, 120 mM lithium sulphate, 60 mM Tris.HCl (pH 7.5), 15% (v/v) polyethylene glycol 400, 5 mM AMPPNP/MgCl₂ for 20 secs, prior to mounting the crystals in a ryan loop, and freezing in a nitrogen gas stream at 100 K. X-ray diffraction data were collected at the SRS, Daresbury, UK and at the European Synchrotron Radiation Facility, Grenoble, France.

Data were collected and these were analysed and processed using the HKL (Otwinowski and Minor, 1997) and CCP4 (CCP4, 1994) program suites. The structure was solved by means of molecular replacement using the coordinates of the ternary complex of the catalytic subunit of murine PKA as a search model (Knighton et al., 1991) with the program CNS (Brünger et al., 1998). The atomic structure was refined using rigid body and least squares refinement with CNS. Model building and analysis was done using O (Jones et al., 1991).

Protein Kinase B Assay

PKB was assayed essentially as described by Andjelkovic et al. (1999) with 30 μM Crosstide (GRPRTSSAEG) as substrate except the protein kinase A inhibitor peptide was not added to the reactions. For peptide stimulation experiments the various peptides were dissolved in water and added to the kinase assay mix prior to adding the PKB protein. Peptides were synthesized by Franz Fischer at the FMI or purchased from Neosystem, Strasburg, France.

Peptides used were:

| | |
|---|---|
| PKB HM-P | GLLELDQRTHFPQFpSYSASIRB |
| PKB HM-D | GLLELDQRTHFPQFDYSASIRE |
| PKB HM-S | GLLELDQRTHFPQFSYSASIRE |
| PKB HM-PF | GLLELDQRTHABQApSYSASIRE |
| PIFtide | REPRILSEEEQEMFRDFDYIADW |
| PIFtide (D->A) | REPRILSEEEQEMFRDFAYIADW |
| PIFtide1 | MFRDFDYIADW |
| PIFtide2 | MFRDFAYIADW |
| PTFtide3 | MFRAFAYIADW |
| PIFtide4 | MARDADYIADW |
| PIFtide5 | MFRDFDATADW |

pS is used to indicate phosphoserine.

All experiments were performed in either duplicate or triplicate.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. The references in the above text and listed below are incorporated by reference.

### REFERENCES

Alessi, D. R. (2001) Discovery of PDK1, one of the missing links in insulin signal transduction. Biochem Soc. Trans 29; 1-14

Alessi, D. R., Andjelkovic, M., Caudwell B. F., Cron, P, Morrice, N., Cohen, P. and Hemmings, B. A. (1996) Mechanism of activation of protein kinase B by insulin and IGF1. EMBO J. 15, 6541-6551

Alessi, D. R., Deak, M., Casamayor, A., Caudwell, F. B., Morrice, N., Norman, D. G., Gaffney, P., Reese, C. B., MacDougall, C. N., Harbison, D., Ashworth, A., and Bownes, M. (1997b) 3-Phosphoinositide-dependent protein kinase-1 (PDK1): structural and functional homology with the Drosophila DSTPK61 kinase. Curr Biol. 7, 776-789.

Alessi, D. R., James, S. R., Downes, C. P., Holmes, A. B., Gaffney, P. R., Reese, C. B., and Cohen, P. (1997a) Characterization of a 3-phosphoinositide-dependent protein kinase which phosphorylates and activates protein kinase Balpha. Curr. Biol. 7, 261-269.

Altomare, D. A., Wan, M., Dubeau, L., Sacmbia, G., Masciullo, V. (1995) Molecular alterations of the Akt2 oncogene in ovarian and breast carcinomas. Int. J. Cancer 64; 280-285

Altschul, S. F., Madden, T. L., Schaffer, A. A., Zhang, Z., Miller, W. and Lipman, D. J. (1997). Gapped BLAST and PSI-BLAST: a new generation of protein database search programs. Nucleic Acids Res., 25 3389-3402.

Andjelkovic, M., Alessi, D. R., Meier, R., Fernandez, A., Lamb, N. J. C., Frech, M., Cron, P., Lucocq, J. M., Hemmings, B. A. (1997) Role of translocation in the activation and function of protein kinase B. J. Biol. Chem. 272; 31515-31524

Andjelkovic, M., Maira, S. M., Cron, P., Parker, P. J., Hemmings, B. A. (1999) Domain swapping used to investigate the mechanism of protein kinase B regulation by 3-phosphoinositide-dependent protein kinase 1 and Ser473 kinase. Mol. Cell. Biol. 19; 5061-5072

Balendran, A., Casamayor, A., Deak, M., Paterson, A., Gaffney, P., Currie, R., Downes, C. P., Alessi, D. R. (1999) PDK1 acquires PDK2 activity in the presence of a synthetic peptide derived from the carboxy terminus of PRK2. Curr. Biol 9; 393-404

Barton, G. J. (1993). ALSCRIPT: a tool to format multiple sequence alignments. Protein Eng., 6, 37-40.

Barton, G. J. (1994). The AMPS package for multiple sequence alignment. Methods Mol. Biol., 25, 327-347.

Belham, C., Wu, S., Avruch, J. (1999) Intracellular signalling: PDK1—a kinase at the hub of things. Curr. Biol 9; R93-96

Bellacosa, A., Testa, J. R., Staal, S. P., and Tsichlis, P. N. (1991) A retroviral oncogene, akt, encoding a serine-threonine kinase containing an SH2-like region. Science 254, 274-277.

Bellacosa, A., Chan, T. O., Ahmed, N. N., Datta, K., Malstrom, S., Stokoe, D., McCormick, F., Feng, J. and Tsichlis, P. (1998). Akt activation by growth factors is a multiple-step process: the role of the PH domain. Oncogene, 17, 313-325.

Biondi, R. M., Cheung, P. C., Casamayor, A., Deak, M., Currie, R. A. and Alessi, D. R. (2000). Identification of a pocket in the PDK1 kinase domain that interacts with PIF and the C-terminal residues of PKA. EMBO J., 19, 979-988.

Biondi, R. M., Kieloch, A., Currie, R. A., Deak, M. and Alessi, D. R. (2001). The PIF-binding pocket in PDK1 is essential for activation of S6K and SGK, but not PKB. EMBO J., 20, 4380-4390.

Bornancin, F. and Parker, P. J. (1997). Phosphorylation of protein kinase C alpha on serine 657 controls the accumulation of active enzyme and contributes to its phosphatase-resistant state. J. Biol. Chem., 272, 3544-3549.

Bossemeyer, D., Engh, R. A., Kinzel, V., Ponstingl, H. and Huber, R. (1993). Phosphotransferase and substrate binding mechanism of the cAMP-dependent protein kinase catalytic subunit from porcine heart as deduced from the 2.0 Å structure of the complex with Mn²⁺ adenylyl imidodiphosphate and inhibitor peptide PKI (5-24). EMBO J., 12, 849-859.

Bradshaw, J. M., Grucza, R. A., Ladbury, J. E. and Waksman, G. (1998). Probing the “two-pronged plug two-holed socket” model for the mechanism of binding of the Src SH2 domain to phosphotyrosyl peptides: a thermodynamic study. Biochemistry, 37, 9083-9090.

Brändén, C. -I. (1980). Relation between structure and function of alpha-beta-proteins. Quarterly Rev. of Biophys. 13, 317-338.

Brazil, D. P. and Hemmings, B. A. (2001) Ten years of PKB signaling: A hard Akt to follow. Trends Biochem. Sci. 26, 657 - 664.

Brodbeck, D., Cron, P., and Hemmings, B. A. (1999) A human protein kinase Bγ with regulatory phosphorylation sites in the activation loop and in the C-terminal hydrophobic domain. J. Biol. Chem., 274, 9133-9136

Brodbeck, D., Hill, M. M. and Hemmings, B. A. (2001). Two splice variants of protein kinase B gamma have different regulatory capacity depending on the presence or absence of the regulatory phosphorylation site serine 472 in the carboxyl-terminal hydrophobic domain. J. Biol. Chem., 276, 29550-29558.

Brünger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., Warren, G. L. (1998). Crystallography and NMR system: A new software suite for macromolecular structure determination. Acta Crystallogr D Biol Crystallogr 54, 905-921

Brünger, A. T., Kurian, J., and Karplus, M. (1987). Crystallographic R factor refinement by molecular dynamics. Science 235, 458-560.

Canagarajah, B. J., Khokhlatchev, A., Cobb, M. H. and Goldsmith, E. J. (1997). Activation mechanism of the MAP kinase ERK2 by dual phosphorylation. Cell, 90, 859-869.

Cantley, L. C. and Neel, B. G. (1999). New insights into tumor suppression: PTEN suppresses tumor formation by restraining the phosphoinositide 3-kinase/AKT pathway. Proc. Natl. Acad. Sci. USA., 96, 4240-4245.

CCP4-Collaborative Computational Project 4. (1994) The CCP4 Suite: Programs for Protein Crystallography. Acta Crystallographica D50, 760-763.

Cheng, J. Q., Godwin, A. K., Bellacosa, A., Taguchi, T., Franke, T. F., Hamilton, T. C., Tsichlis, P. N., and Testa, J. R. (1992) AKT2, a putative oncogene encoding a member of a subfamily of protein-serine/threonine kinases, is amplified in human ovarian carcinomas. Proc. Natl. Acad. Sci. U S A. 89, 9267-9271.

Cheng, J. Q., Ruggeri, B., Klein, W. M., Sonoda, G., Altomare, D. A., Watson, D. K., and Testa, J. R. (1996) Amplification of AKT2 in human pancreatic cells and inhibition of AKT2 expression and tumorigenicity by antisense RNA. Proc. Natl. Acad. Sci. U S A. 93, 3636-3641.

Chothia, C. and Lesk, A. M. (1986). The relation between the divergence of sequence and structure in proteins. EMBO J., 5, 823-826.

Coffer, P. J., and Woodgett, J. R. (1991) Molecular cloning and characterisation of a novel putative protein-serine kinase related to the cAMP-dependent and protein kinase C families. Eur. J. Biochem. 201, 475-481.

Datta, S. R., Brunet, A. and Greenberg, M. E. (1999) Cellular survival: a play in three Akts. Genes and Development 13, 2903-2927

Doublie, S. (1997). Preparation of Selenomethyionyl Proteins for Phase Determination. Methods in Enzymology 276, 523-530.

Esnouf, R. M. (1997). An extensively modified version of MOLSCRIPT that includes greatly enhanced coloring capabilities. J. Mol. Graph. Model., 15, 132-134.

Etchebehere, L. C., Van Bemmelen, M. X., Anjard, C., Traincard, F., Assemat, K., Reymond, C. and Veron, M. (1997). The catalytic subunit of Dictyostelium cAMP-dependent protein kinase—role of the N-terminal domain and of the C-terminal residues in catalytic activity and stability. Eur. J. Biochem., 248, 820-826.

Evans, P. R. (1997). Scaling of MAD data. In Recent Advances in Phasing (ed. K. S. Wilson, G. Davies, A. W. Ashton and S. Bailey), pp. 97-102. Council for the Central Laboratory of the Research Councils Daresbury Laboratory, Daresbury, UK.

Filippa, N., Sable, C. L., Hemmings, B. A., Van Obberghen, E. (2000) Effect of phosphoinositide-dependent kinase 1 on protein kinase B translocation and its subsequent activation. Mol Cell Biol. 20(15);5712-21.

Frodin, M., Jensen, C. J., Merienne, K. and Gammeltoft, S. (2000). A phosphoserine-regulated docking site in the protein kinase RSK2 that recruits and activates PDK1. EMBO J., 19, 2924-34.

Galetic, I., Andjelkovic, M., Meier, R., Brodbeck, D., Park J. and Hemmings, B. A. (1999) Mechanism of protein kinase B activation by Insulin/IGF1 revealed by specific inhibitors of phosphoinositide 3-kinase-significance for diabetes and cancer. Pharmacology and Therapeutics, Academic Press, 82, 409-425

Hendrickson W. A. (1991). Determination of macromolecular structures from anomalous diffraction of synchrotron radiation. Science 254, 51-58.

Hill, M. M. and Hemmings, B. A. (2002). Analysis of protein kinase B/Akt. Methods Enzymol. 345, 448-463.

Holm, L. and Sander, C. (1998). Touring protein fold space with Dali/FSSP. Nucleic Acids Research 26, 316-319.

Hubbard, S. R.(1997). Crystal structure of the activated insulin receptor tyrosine kinase in complex with peptide substrate and ATP analog. EMBO J., 16, 5572-5581.

Hubbard, S. R., Wei, L., Ellis, L. and Hendrickson, W. A. (1994). Crystal structure of the tyrosine kinase domain of the human insulin receptor. Nature, 372, 746-754.

Jeffrey, P. D., Russo, A. A., Polyak, K., Gibbs, E., Hurwitz, J., Massague, J. and Pavletich, N. P. (1995). Mechanism of CDK activation revealed by the structure of a cyclinA-CDK2 complex. Nature, 376, 313-320.

Johnson, L. N., Noble, M. E., Owen, D. J. (1996). Active and inactive protein kinases: structural basis for regulation. Cell, 85, 149-158.

Jones, P. F., Jakubowicz, T., Pitossi, F. J., Maurer, F. and Hemmings, B. A. (1991a) Molecular cloning and identification of a novel serine/threonine protein kinase (PKB). Proc. Natl. Acad. Sci. USA 88, 4171-4175.

Jones, T. A., Zou, J. Y., Cowan, S. W. and Kjeldgaard, M. (1991). Improved methods for building protein models in electron density maps and the location of errors in these models. Acta Crystallographica, A47, 110-119.

Jones, T. A., Zou, J. Y., Cowan, S. W. and Kjeldgaard, M. (1991b) Acta Crystallographica, Sect. A 50, 157-60.

Kallen, R. G. & Jencks. (1966). The mechanism of the condensation of formaldehyde with tetrahydrofolic acid. Journal of Biological Chemistry 241, 5851-5863.

Keranen, L. M., Dutil, E. M. and Newton, A. C. (1995). Protein kinase C is regulated in vivo by three functionally distinct phosphorylations. Curr. Biol., 5, 1394-1403.

Knighton, D. R., Kan, C. C., Howland, E., Janson, C. A., Hostomska, Z., Welsh, K. M. and Matthews, D. A. (1994). Structure of and kinetic channelling in bifunctional dihydrofolate reductase-thymidylate synthase. Nat. Struct. Biol. 1, 186-194.

Knighton, D. R., Zheng, J. H., Ten Eyck, L. F., Ashford, V. A., Xuong, N. H., Taylor, S. S. and Sowadski, J. M. (1991a). Crystal structure of the catalytic subunit of cyclic adenosine monophosphate-dependent protein kinase. Science, 253, 407-414.

Knighton, D. R., Zheng, J. H., Ten Eyck, L. F., Xuong, N. H., Taylor, S. S. and Sowadski, J. M. (1991b). Structure of a peptide inhibitor bound to the catalytic subunit of cyclic adenosine monophosphate-dependent protein kinase. Science, 253, 414-420.

Kobayashi, T. and Cohen, P. (1999). Activation of serum- and glucocorticoid-regulated protein kinase by agonists that activate phosphatidylinositide 3-kinase is mediated by 3-phosphoinositide-dependent protein kinase-1 (PDK1) and PDK2. Biochem. J., 339, 319-328.

La Fortelle, E. de and Bricogne, G. (1997). Maximum-likelihood heavy-atom parameter refinement for multiple isomorphous replacement and multiwavelength anomalous diffraction methods. Methods in Enzymology 276, 472-494.

Laskowski, R. A. (1993). PROCHECK: a program to check the stereochemical quality of preotein structures. J. Appl. Cryst. 26, 283-291.

Leslie, A. G. W. (1992). In Joint CCP4 and EESF-EACMB Newsletter on Protein Crystallography, vol. 26, Warrington, Daresbury Laboratory.

Li, J., Yen, C., Liaw, D., Podsypanina, K., Bose, S., Wang, S. I., Puc, J., Miliaresis, C., Rodgers, L., McCombie, R., Bigner, S. H., Giovanella, B. C., Ittmann, M., Tycko, B., Hibshoosh, H., Wigler, M. H., Parsons, R. (1997) PTEN, a putative protein tyrosine phosphatase gene mutated in human brain, breast, and prostate cancer. Science 275(5308): 1943-1947

Lowe, E. D., Noble, M. E., Skamnaki, V. T., Oikonomakos, N. G., Owen, D. J. and Johnson, L. N. (1997). The crystal structure of a phosphorylase kinase peptide substrate complex: kinase substrate recognition. EMBO J., 16, 6646-6658

Maehama T., and Dixon J. E. (1998) The tumor suppressor, PTEN/MMAC1, dephosphorylates the lipid second messenger, phosphatidylinositol 3,4,5-trisphosphate. J. Biol. Chem. 273, 13375-13378.

Maehama, T., Taylor, G. S., Dixon, J. E. (2001) PTEN and mytubularin: novel phosphoinositide phosphatase. Annu. Rev. Biochem 70; 247-279

Maira, S. M., Galetic, I., Brazil, D. P., Kaech, S., Ingley, E., Thelen, M. and Hemmings, B. A. (2001). Carboxyl-terminal modulator protein (CTMP), a negative regulator of PKB/Akt and v-Akt at the plasma membrane. Science, 294, 374-380.

Mayo, L. D. and Donner, D. B. (2001). A phosphatidylinositol 3-kinase/Akt pathway promotes translocation of Mdm2 from the cytoplasm to the nucleus. Proc. Natl. Acad. Sci. USA., 98, 11598-11603.

Meier, R., Alessi, D. R., Cron, P., Andjelkovic, M., and Hemmings, B. A. (1997). Mitogenic activation, phosphorylation and nuclear translocation of protein kinase B beta. J. Biol. Chem., 272, 30491-7.

Merit, E. A. and Murphy, M. E. P. (1994). Raster3D Version 2.0. A program for photorealistic molecular graphics. Acta Crystrallogr. D., 50, 869-873.

Miwa, W., Yasuda, J., Murakami, Y., Yashima, K., Sugamo, K. Sekine, T., Kono, A., Egawa, S., Yamaguchi, K. Hyayshizaki, Y. and Sekiya, T. (1996) Isolation of DNA sequences amplified at chromosome 19q13.1-q13.2 including the Akt2 locus in human pancreatic cancer. Biochem. Biophys. Res. Commun. 225, 968-974.

Murshudov, G. N., Vagin, A. A. and Dodson, E. J. (1997). Refinement of macromolecular structures by the maximum-likelihood method. Acta Crystallographica, 1997 D53, 240-255.

Myers, M. P., Pass, I., Batty, I. H., Van der Kaay, J., Stolarov, J. P., Hemmings, B. A., Wigler, M. H., Downes, C. P., and Tonks, N. K. (1998)

The lipid phosphatase activity of PTEN is critical for its tumor suppressor function. Proc. Natl. Acad. Sci. USA 95, 13513-13518.

Nicholls, A., Sharp, K. A. and Honig, B. (1991). Protein folding and association: insights from the interfacial and thermodynamic properties of hydrocarbons. Proteins, 11, 281-296.

Obata, T., Yaffe, M. B., Leparc, G. G., Piro, E. T., Maegawa, H., Kashiwagi, A., Kikkawa, R. and Cantley, L. C. (2000). Peptide and protein library screening defines optimal substrate motifs for AKT/PKB. J. Biol. Chem., 275, 36108-36115.

Otwinowski, Z. and Minor, W. (1997) Processing of X-ray Diffraction Data Collected in Oscillation Mode. Methods in Enzymology, 276, 307-326.

Parekh, D. B., Ziegler, W. and Parker, P. J. (2000). Multiple pathways control protein kinase C phosphorylation. EMBO J., 19, 496-503.

Pearson, R. B., Dennis, P. B., Han, J. W., Williamson, N. A., Kozma, S. C., Wettenhall, R. E. and Thomas, G. (1995). The principal target of rapamycin-induced p70s6k inactivation is a novel phosphorylation site within a conserved hydrophobic domain. EMBO J. 14, 5279-5287.

Pflugrath, J. W. (1999). The finer things in X-ray diffraction data collection. Acta Crystallographica D55, 1718-1725.

Pullen, N., Dennis, P. B., Andjelkovic, M., Dufner, A., Kozma, S. C., Hemmings, B. A. and Thomas, G. (1998). Science, 279, 707-710.

Scarsdale, J. N., Kazanina, G., Radaev, S., Schirch, V., and Wright, H. T. (1999). Crystal structure of rabbit cytosolic serine hydroxymethyltransferase at 2.8 Å resolution: mechanistic implications. Biochemistry 38, 8347-8358.

Schirch, V., and Strong, W. B. (1989) Interaction of folylpolyglutamates with enzymes in one-carbon metabolism. Arch. Biochem. Biophys. 269, 371-380.

Shevchenko, A., Wilm, M., Vorm, O. and Mann, M. (1996). Mass spectrometric sequencing of proteins silver-stained polyacrylamide gel. Anal. Chem. 68, 850-858.

Shoji, S., Titani, K., Demaille, J. G. and Fischer, E. H. (1979). Sequence of two phosphorylated sites in the catalytic subunit of bovine cardiac muscle adenosine 3′:5′-monophosphate-dependent protein kinase. J. Biol. Chem., 254, 6211-6214.

Shoji, S., Ericsson, L. H., Walsh, K. A., Fischer, E. H., and Titani, K. (1983). Amino acid sequence of the catalytic subunit of bovine type II adenosine cyclic 3′,5′-phosphate dependent protein kinase. Biochemistry, 19, 3702-9.

Sicheri, F., Moarefi, I. and Kuriyan, J. (1997). Crystal structure of the Src family tyrosine kinase Hck. Nature, 385, 602-609.

Staal, S. P. (1987) Molecular cloning of the akt oncogene and its human homologues AKT1 and AKT2, amplification of AKT1 in a primary human gastric adenocarcinoma. Proc. Natl. Acad. Sci. U S A. 84, 5034-5037.

Staal, S. P., Hartley, J. W. and Rowe, W. P. (1977). Isolation of transforming murine leukemia viruses from mice with a high incidence of spontaneous lymphoma. Proc. Natl. Acad. Sci. USA, 74, 3065-3067.

Stokoe, D., Stephens, L. R., Copeland, T., Gaffney, P. R., Reese, C. B., Painter, G. F., Holmes, A. B., McCormick, F., and Hawkins, P. T. (1997) Dual role of phosphatidylinositol-3,4,5-trisphosphate in the activation of protein kinase B. Science 277, 567-570.

Teller, J. H., Powers, S. G. and Snell, E. E. (1976). Ketopantoate hydroxymethyltransferase. I. Purification and role in pantothenate biosynthesis. J. Biol. Chem. 251, 3780-3785.

Testa, R. and Bellacosa, A. AKT plays a central role in tumorigenesis. Proc. Natl. Acad. Sci. USA., 98, 10983-10985.

Thompson, J. D., Higgins, D. G. and Gibson, T. J. (1994). CLUSTAL W: improving the sensitivity of progressive multiple sequence alignment through sequence weighting, positions-specific gap penalties and weight matrix choice. Nucleic Acids Research 22, 4673-4680.

Vanhaesebroeck, B. and Alessi, D. R. (2000). The PI3K-PDK1 connection: more than just a road to PKB. Biochem. J., 346, 561-576.

Weeks, C. M., Detitta, G. T., Hauptman, H. A., Thuman, P. and Miller, R. (1994). Structure solution by minimal function phase refinement and Fourier filtering II. Implementation and applications. Acta Crystallographica A50, 210-220.

Williams, M. R., Arthur, J. S., Balendran, A., van der Kaay, J., Poli, V., Cohen, P. and Alessi D. R. (2000). The role of 3-phosphoinositide-dependent protein kinase 1 in activating AGC kinases defined in embryonic stem cells. Curr. Biol., 10, 439-448.

Wilm, M. and Mann, M. (1996). Analytical properties of the nanoelectrospray ion source. Anal. Chem., 68, 1-8.

Wybenga-Groot, L. E., Baskin, B., Ong, S. H., Tong, J., Pawson, T. and Sicheri, F. (2001). Structural basis for autoinhibition of the Ephb2 receptor tyrosine kinase by the unphosphorylated juxtamembrane region. Cell, 106, 745-757

Xu, W., Harrison, S. C. and Eck, M. J. (1997). Three-dimensional structure of the tyrosine kinase c-Src. Nature, 385, 595-602

Yonemoto, W., McGlone, M. L., Grant, B. and Taylor, S. S. (1997). Autophosphorylation of the catalytic subunit of cAMP-dependent protein kinase in Escherichia coli. Protein Eng., 10, 915-925.

Zhou, B. P., Liao, Y., Xia, W., Spohn, B., Lee, M. H. and Hung, M. C. (2001). Cytoplasmic localization of p21Cip1/WAF1 by Akt-induced phosphorylation in HER-2/neu-overexpressing cells. Nat. Cell Biol., 3, 245-252.

TABLE 2
Coordinate data for pΔPH-PKBβ-ΔC (first batch)

REMARK coordinates from restrained individual B-factor refinement
REMARK refinement resolution: 500.0-2.8 A
REMARK starting r = 0.3586 free_r = 0.4460
REMARK final r = 0.3498 free_r = 0.4274
REMARK B rmsd for bonded mainchain atoms = 1.346 target = 1.5
REMARK B rmsd for bonded sidechain atoms = 1.595 target = 2.0
REMARK B rmsd for angle mainchain atoms = 2.356 target = 2.0
REMARK B rmsd for angle sidechain atoms = 2.450 target = 2.5
REMARK rweight = 0.1000 (with wa = 7.46871)
REMARK target = mlf steps = 30
REMARK sg = P4 (1) 2 (1) 2 a = 149.33 b = 149.33 c = 39.371 alpha = 90 beta = 90
gamma = 90
REMARK parameter file 1: CNS_TOPPAR: protein_rep.param
REMARK molecular structure file: generate_easy.mtf
REMARK input coordinates: anneal_1.pdb
REMARK reflection file = pkb12-free.fob
REMARK ncs = none
REMARK B-correction resolution: 6.0-2.8
REMARK initial B-factor correction applied to fobs:
REMARK B11 = −3.842 B22 = −3.842 B33 = 7.684
REMARK B12 = 0.000 B13 = 0.000 B23 = 0.000
REMARK B-factor correction applied to coordinate array B: −3.876
REMARK bulk solvent: density level = 0.312838 e/A\hat{ }3, B-factor = 24.4035 A\hat{ }2
REMARK reflections with |Fobs|/sigma_F < 0.0 rejected
REMARK reflections with |Fobs| > 10000 * rms (Fobs) rejected
REMARK theoretical total number of refl. in resol. range: 11510 (100.0%)
REMARK number of unobserved reflections (no entry or |F| = 0): 116 (1.0%)
REMARK number of reflections rejected: 0 (0.0%)
REMARK total number of reflections used: 11394 (99.0%)
REMARK number of reflections in working set: 10537 (91.5%)
REMARK number of reflections in test set: 857 (7.4%)
CRYST1 149.330 149.330 39.371 90.00 90.00 90.00 P 41 21 2
REMARK FILENAME = “bindividual.pdb”
REMARK DATE: 08-Aug-01 09:23:49
REMARK VERSION: 1.0

| | | | | | | | | | | | |
|---|---|---|---|---|---|---|---|---|---|---|---|
| ATOM | 1 | CB | MET | A | 149 | −33.122 | 75.969 | −56.453 | 1.00 | 41.57 | A |
| ATOM | 2 | CG | MET | A | 149 | −31.773 | 76.431 | −55.956 | 1.00 | 39.81 | A |
| ATOM | 3 | SD | MET | A | 149 | −30.713 | 77.256 | −57.141 | 1.00 | 32.34 | A |
| ATOM | 4 | CE | MET | A | 149 | −29.260 | 76.531 | −56.686 | 1.00 | 39.03 | A |
| ATOM | 5 | C | MET | A | 149 | −35.447 | 76.466 | −57.048 | 1.00 | 48.00 | A |
| ATOM | 6 | O | MET | A | 149 | −35.763 | 76.754 | −58.196 | 1.00 | 49.15 | A |
| ATOM | 7 | N | MET | A | 149 | −34.473 | 77.511 | −55.030 | 1.00 | 44.08 | A |
| ATOM | 8 | CA | MET | A | 149 | −34.207 | 77.047 | −56.423 | 1.00 | 45.80 | A |
| ATOM | 9 | N | ASN | A | 150 | −36.141 | 75.634 | −56.279 | 1.00 | 50.82 | A |
| ATOM | 10 | CA | ASN | A | 150 | −37.346 | 74.969 | −56.742 | 1.00 | 52.03 | A |
| ATOM | 11 | CB | ASN | A | 150 | −37.762 | 73.906 | −55.729 | 1.00 | 52.70 | A |
| ATOM | 12 | CG | ASN | A | 150 | −38.459 | 72.725 | −56.374 | 1.00 | 54.27 | A |
| ATOM | 13 | OD1 | ASN | A | 150 | −38.811 | 71.759 | −55.703 | 1.00 | 56.34 | A |
| ATOM | 14 | ND2 | ASN | A | 150 | −38.660 | 72.796 | −57.686 | 1.00 | 53.74 | A |
| ATOM | 15 | C | ASN | A | 150 | −38.499 | 75.938 | −57.007 | 1.00 | 52.68 | A |
| ATOM | 16 | O | ASN | A | 150 | −39.639 | 75.522 | −57.181 | 1.00 | 55.34 | A |
| ATOM | 17 | N | ASP | A | 151 | −38.206 | 77.231 | −57.029 | 1.00 | 51.91 | A |
| ATOM | 18 | CA | ASP | A | 151 | −39.229 | 78.220 | −57.328 | 1.00 | 51.33 | A |
| ATOM | 19 | CB | ASP | A | 151 | −39.183 | 79.384 | −56.328 | 1.00 | 52.09 | A |
| ATOM | 20 | CG | ASP | A | 151 | −39.604 | 78.973 | −54.930 | 1.00 | 51.99 | A |
| ATOM | 21 | OD1 | ASP | A | 151 | −40.425 | 78.044 | −54.831 | 1.00 | 53.76 | A |
| ATOM | 22 | OD2 | ASP | A | 151 | −39.133 | 79.583 | −53.940 | 1.00 | 50.00 | A |
| ATOM | 23 | C | ASP | A | 151 | −38.956 | 78.744 | −58.738 | 1.00 | 50.85 | A |
| ATOM | 24 | O | ASP | A | 151 | −39.765 | 79.468 | −59.316 | 1.00 | 50.83 | A |
| ATOM | 25 | N | PHE | A | 152 | −37.815 | 78.350 | −59.293 | 1.00 | 50.55 | A |
| ATOM | 26 | CA | PHE | A | 152 | −37.415 | 78.805 | −60.619 | 1.00 | 51.05 | A |
| ATOM | 27 | CB | PHE | A | 152 | −36.128 | 79.643 | −60.533 | 1.00 | 50.53 | A |
| ATOM | 28 | CG | PHE | A | 152 | −36.095 | 80.596 | −59.380 | 1.00 | 49.74 | A |
| ATOM | 29 | CD1 | PHE | A | 152 | −36.885 | 81.730 | −59.374 | 1.00 | 48.38 | A |
| ATOM | 30 | CD2 | PHE | A | 152 | −35.288 | 80.332 | −58.277 | 1.00 | 51.27 | A |
| ATOM | 31 | CE1 | PHE | A | 152 | −36.876 | 82.589 | −58.284 | 1.00 | 49.17 | A |
| ATOM | 32 | CE2 | PHE | A | 152 | −35.268 | 81.182 | −57.181 | 1.00 | 49.87 | A |
| ATOM | 33 | CZ | PHE | A | 152 | −36.062 | 82.309 | −57.182 | 1.00 | 49.50 | A |
| ATOM | 34 | C | PHE | A | 152 | −37.170 | 77.696 | −61.638 | 1.00 | 50.08 | A |
| ATOM | 35 | O | PHE | A | 152 | −36.805 | 76.573 | −61.287 | 1.00 | 50.15 | A |
| ATOM | 36 | N | ASP | A | 153 | −37.358 | 78.059 | −62.906 | 1.00 | 49.19 | A |
| ATOM | 37 | CA | ASP | A | 153 | −37.138 | 77.185 | −64.050 | 1.00 | 47.34 | A |
| ATOM | 38 | CB | ASP | A | 153 | −38.375 | 77.153 | −64.946 | 1.00 | 46.91 | A |
| ATOM | 39 | CG | ASP | A | 153 | −39.232 | 75.934 | −64.696 | 1.00 | 47.34 | A |
| ATOM | 40 | OD1 | ASP | A | 153 | −38.695 | 74.822 | −64.883 | 1.00 | 47.91 | A |
| ATOM | 41 | OD2 | ASP | A | 153 | −40.419 | 76.075 | −64.308 | 1.00 | 46.26 | A |
| ATOM | 42 | C | ASP | A | 153 | −35.956 | 77.758 | −64.817 | 1.00 | 47.18 | A |
| ATOM | 43 | O | ASP | A | 153 | −35.804 | 78.972 | −64.914 | 1.00 | 48.16 | A |
| ATOM | 44 | N | TYR | A | 154 | −35.118 | 76.884 | −65.353 | 1.00 | 45.64 | A |
| ATOM | 45 | CA | TYR | A | 154 | −33.937 | 77.306 | −66.088 | 1.00 | 43.40 | A |
| ATOM | 46 | CB | TYR | A | 154 | −32.808 | 76.329 | −65.799 | 1.00 | 43.14 | A |
| ATOM | 47 | CG | TYR | A | 154 | −31.640 | 76.382 | −66.752 | 0.00 | 40.46 | A |
| ATOM | 48 | CD1 | TYR | A | 154 | −30.746 | 77.454 | −66.755 | 1.00 | 41.17 | A |
| ATOM | 49 | CE1 | TYR | A | 154 | −29.610 | 77.429 | −67.559 | 1.00 | 39.92 | A |
| ATOM | 50 | CD2 | TYR | A | 154 | −31.373 | 75.309 | −67.586 | 1.00 | 40.00 | A |
| ATOM | 51 | CE2 | TYR | A | 154 | −30.247 | 75.283 | −68.380 | 1.00 | 39.95 | A |
| ATOM | 52 | CZ | TYR | A | 154 | −29.370 | 76.333 | −68.355 | 0.00 | 39.99 | A |
| ATOM | 53 | OH | TYR | A | 154 | −28.211 | 76.231 | −69.061 | 1.00 | 39.63 | A |
| ATOM | 54 | C | TYR | A | 154 | −34.179 | 77.395 | −67.580 | 1.00 | 43.66 | A |
| ATOM | 55 | O | TYR | A | 154 | −34.638 | 76.439 | −68.191 | 1.00 | 44.55 | A |
| ATOM | 56 | N | LEU | A | 155 | −33.855 | 78.545 | −68.162 | 1.00 | 44.30 | A |
| ATOM | 57 | CA | LEU | A | 155 | −34.044 | 78.773 | −69.592 | 1.00 | 44.60 | A |
| ATOM | 58 | CB | LEU | A | 155 | −34.840 | 80.063 | −69.823 | 1.00 | 43.16 | A |
| ATOM | 59 | CG | LEU | A | 155 | −36.155 | 80.228 | −69.054 | 1.00 | 40.74 | A |
| ATOM | 60 | CD1 | LEU | A | 155 | −36.986 | 81.300 | −69.725 | 1.00 | 40.57 | A |
| ATOM | 61 | CD2 | LEU | A | 155 | −36.920 | 78.926 | −69.029 | 1.00 | 38.72 | A |
| ATOM | 62 | C | LEU | A | 155 | −32.720 | 78.833 | −70.351 | 1.00 | 45.07 | A |
| ATOM | 63 | O | LEU | A | 155 | −32.524 | 78.097 | −71.315 | 1.00 | 44.89 | A |
| ATOM | 64 | N | LYS | A | 156 | −31.813 | 79.711 | −69.942 | 1.00 | 46.46 | A |
| ATOM | 65 | CA | LYS | A | 156 | −30.515 | 79.774 | −70.617 | 1.00 | 47.97 | A |
| ATOM | 66 | CB | LYS | A | 156 | −30.614 | 80.583 | −71.918 | 1.00 | 48.50 | A |
| ATOM | 67 | CG | LYS | A | 156 | −31.049 | 82.031 | −71.766 | 1.00 | 50.02 | A |
| ATOM | 68 | CD | LYS | A | 156 | −30.694 | 82.860 | −73.009 | 1.00 | 50.68 | A |
| ATOM | 69 | CE | LYS | A | 156 | −31.286 | 82.290 | −74.301 | 1.00 | 50.37 | A |
| ATOM | 70 | NZ | LYS | A | 156 | −30.608 | 81.038 | −74.771 | 1.00 | 49.95 | A |
| ATOM | 71 | C | LYS | A | 156 | −29.386 | 80.338 | −69.768 | 1.00 | 47.79 | A |
| ATOM | 72 | O | LYS | A | 156 | −29.605 | 80.795 | −68.645 | 1.00 | 46.87 | A |
| ATOM | 73 | N | LEU | A | 157 | −28.168 | 80.279 | −70.298 | 1.00 | 47.58 | A |
| ATOM | 74 | CA | LEU | A | 157 | −27.035 | 80.847 | −69.581 | 1.00 | 47.46 | A |
| ATOM | 75 | CB | LEU | A | 157 | −25.714 | 80.138 | −69.897 | 1.00 | 47.94 | A |
| ATOM | 76 | CG | LEU | A | 157 | −24.586 | 80.672 | −68.989 | 1.00 | 47.41 | A |
| ATOM | 77 | CD1 | LEU | A | 157 | −24.787 | 80.142 | −67.583 | 1.00 | 46.53 | A |
| ATOM | 78 | CD2 | LEU | A | 157 | −23.210 | 80.264 | −69.507 | 1.00 | 47.40 | A |
| ATOM | 79 | C | LEU | A | 157 | −26.929 | 82.278 | −70.050 | 1.00 | 47.03 | A |
| ATOM | 80 | O | LEU | A | 157 | −27.089 | 82.556 | −71.234 | 1.00 | 46.03 | A |
| ATOM | 81 | N | LEU | A | 158 | −26.678 | 83.186 | −69.118 | 1.00 | 47.66 | A |
| ATOM | 82 | CA | LEU | A | 158 | −26.543 | 84.588 | −69.453 | 1.00 | 48.86 | A |
| ATOM | 83 | CB | LEU | A | 158 | −27.611 | 85.417 | −68.728 | 1.00 | 48.65 | A |
| ATOM | 84 | CG | LEU | A | 158 | −28.816 | 85.910 | −69.541 | 1.00 | 48.13 | A |
| ATOM | 85 | CD1 | LEU | A | 158 | −29.828 | 86.568 | −68.627 | 1.00 | 47.43 | A |
| ATOM | 86 | CD2 | LEU | A | 158 | −28.351 | 86.900 | −70.587 | 1.00 | 47.99 | A |
| ATOM | 87 | C | LEU | A | 158 | −25.149 | 85.075 | −69.088 | 1.00 | 50.00 | A |
| ATOM | 88 | O | LEU | A | 158 | −24.672 | 86.056 | −69.637 | 1.00 | 51.27 | A |
| ATOM | 89 | N | GLY | A | 159 | −24.485 | 84.382 | −68.174 | 1.00 | 51.99 | A |
| ATOM | 90 | CA | GLY | A | 159 | −23.148 | 84.804 | −67.788 | 1.00 | 54.26 | A |
| ATOM | 91 | C | GLY | A | 159 | −22.406 | 83.946 | −66.772 | 1.00 | 55.56 | A |
| ATOM | 92 | O | GLY | A | 159 | −22.954 | 83.537 | −65.752 | 1.00 | 54.77 | A |
| ATOM | 93 | N | LYS | A | 160 | −21.142 | 83.673 | −67.060 | 1.00 | 56.79 | A |
| ATOM | 94 | CA | LYS | A | 160 | −20.318 | 82.881 | −66.161 | 1.00 | 58.77 | A |
| ATOM | 95 | CB | LYS | A | 160 | −20.105 | 81.468 | −66.722 | 1.00 | 59.06 | A |
| ATOM | 96 | CG | LYS | A | 160 | −21.388 | 80.619 | −66.769 | 1.00 | 60.55 | A |
| ATOM | 97 | CD | LYS | A | 160 | −21.137 | 79.170 | −67.282 | 1.00 | 60.33 | A |
| ATOM | 98 | CE | LYS | A | 160 | −22.308 | 78.198 | −66.944 | 1.00 | 58.20 | A |
| ATOM | 99 | NZ | LYS | A | 160 | −22.193 | 76.854 | −67.615 | 1.00 | 56.57 | A |
| ATOM | 100 | C | LYS | A | 160 | −18.980 | 83.576 | −65.942 | 1.00 | 59.57 | A |
| ATOM | 101 | O | LYS | A | 160 | −18.602 | 84.484 | −66.679 | 1.00 | 58.96 | A |
| ATOM | 102 | N | GLY | A | 161 | −18.271 | 83.155 | −64.907 | 1.00 | 60.56 | A |
| ATOM | 103 | CA | GLY | A | 161 | −16.987 | 83.759 | −64.612 | 1.00 | 61.18 | A |
| ATOM | 104 | C | GLY | A | 161 | −16.312 | 83.045 | −63.463 | 1.00 | 60.79 | A |
| ATOM | 105 | O | GLY | A | 161 | −16.654 | 81.903 | −63.144 | 1.00 | 60.23 | A |
| ATOM | 106 | N | THR | A | 162 | −15.346 | 83.716 | −62.846 | 1.00 | 60.89 | A |
| ATOM | 107 | CA | THR | A | 162 | −14.638 | 83.129 | −61.724 | 1.00 | 61.12 | A |
| ATOM | 108 | CB | THR | A | 162 | −13.312 | 83.839 | −61.475 | 1.00 | 61.60 | A |
| ATOM | 109 | OG1 | THR | A | 162 | −12.567 | 83.870 | −62.698 | 1.00 | 62.30 | A |
| ATOM | 110 | CG2 | THR | A | 162 | −12.502 | 83.097 | −60.403 | 1.00 | 61.02 | A |
| ATOM | 111 | C | THR | A | 162 | −15.498 | 83.253 | −60.483 | 1.00 | 60.80 | A |
| ATOM | 112 | O | THR | A | 162 | −15.849 | 82.247 | −59.855 | 1.00 | 60.74 | A |
| ATOM | 113 | N | PHE | A | 163 | −15.841 | 84.492 | −60.139 | 1.00 | 60.04 | A |
| ATOM | 114 | CA | PHE | A | 163 | −16.668 | 84.749 | −58.966 | 1.00 | 59.38 | A |
| ATOM | 115 | CB | PHE | A | 163 | −16.376 | 86.138 | −58.388 | 1.00 | 59.76 | A |
| ATOM | 116 | CG | PHE | A | 163 | −15.082 | 86.225 | −57.631 | 1.00 | 59.50 | A |
| ATOM | 117 | CD1 | PHE | A | 163 | −14.949 | 87.111 | −56.572 | 1.00 | 59.24 | A |
| ATOM | 118 | CD2 | PHE | A | 163 | −13.992 | 85.444 | −57.989 | 1.00 | 59.94 | A |
| ATOM | 119 | CE1 | PHE | A | 163 | −13.761 | 87.223 | −55.883 | 1.00 | 59.00 | A |
| ATOM | 120 | CE2 | PHE | A | 163 | −12.793 | 85.550 | −57.304 | 1.00 | 60.58 | A |
| ATOM | 121 | CZ | PHE | A | 163 | −12.679 | 86.444 | −56.246 | 1.00 | 59.90 | A |
| ATOM | 122 | C | PHE | A | 163 | −18.164 | 84.623 | −59.228 | 1.00 | 58.25 | A |
| ATOM | 123 | O | PHE | A | 163 | −18.913 | 85.592 | −59.086 | 1.00 | 57.44 | A |
| ATOM | 124 | N | GLY | A | 164 | −18.594 | 83.424 | −59.611 | 1.00 | 56.59 | A |
| ATOM | 125 | CA | GLY | A | 164 | −20.004 | 83.207 | −59.857 | 1.00 | 53.58 | A |
| ATOM | 126 | C | GLY | A | 164 | −20.407 | 82.857 | −61.271 | 1.00 | 50.76 | A |
| ATOM | 127 | O | GLY | A | 164 | −19.651 | 82.255 | −62.025 | 1.00 | 50.72 | A |
| ATOM | 128 | N | LYS | A | 165 | −21.630 | 83.244 | −61.610 | 1.00 | 48.90 | A |
| ATOM | 129 | CA | LYS | A | 165 | −22.213 | 82.996 | −62.918 | 1.00 | 47.11 | A |
| ATOM | 130 | CB | LYS | A | 165 | −22.289 | 81.501 | −63.176 | 1.00 | 46.63 | A |
| ATOM | 131 | CG | LYS | A | 165 | −23.392 | 80.810 | −62.389 | 1.00 | 46.17 | A |
| ATOM | 132 | CD | LYS | A | 165 | −23.317 | 79.301 | −62.504 | 1.00 | 44.60 | A |
| ATOM | 133 | CE | LYS | A | 165 | −23.176 | 78.907 | −63.939 | 1.00 | 43.03 | A |
| ATOM | 134 | NZ | LYS | A | 165 | −24.096 | 79.748 | −64.741 | 1.00 | 41.76 | A |
| ATOM | 135 | C | LYS | A | 165 | −23.626 | 83.559 | −62.842 | 1.00 | 46.26 | A |
| ATOM | 136 | O | LYS | A | 165 | −24.181 | 83.676 | −61.750 | 1.00 | 47.67 | A |
| ATOM | 137 | N | VAL | A | 166 | −24.206 | 83.907 | −63.984 | 1.00 | 43.14 | A |
| ATOM | 138 | CA | VAL | A | 166 | −25.561 | 84.449 | −64.018 | 1.00 | 40.38 | A |
| ATOM | 139 | CB | VAL | A | 166 | −25.569 | 85.950 | −64.489 | 1.00 | 40.92 | A |
| ATOM | 140 | CG1 | VAL | A | 166 | −26.995 | 86.420 | −64.769 | 1.00 | 39.28 | A |
| ATOM | 141 | CG2 | VAL | A | 166 | −24.934 | 86.843 | −63.414 | 1.00 | 39.18 | A |
| ATOM | 142 | C | VAL | A | 166 | −26.416 | 83.604 | −64.948 | 1.00 | 37.94 | A |
| ATOM | 143 | O | VAL | A | 166 | −25.972 | 83.204 | −66.013 | 1.00 | 36.79 | A |
| ATOM | 144 | N | ILE | A | 167 | −27.644 | 83.329 | −64.526 | 1.00 | 36.83 | A |
| ATOM | 145 | CA | ILE | A | 167 | −28.570 | 82.523 | −65.312 | 1.00 | 35.64 | A |
| ATOM | 146 | CB | ILE | A | 167 | −28.961 | 81.213 | −64.575 | 1.00 | 35.37 | A |
| ATOM | 147 | CG2 | ILE | A | 167 | −29.423 | 80.176 | −65.560 | 1.00 | 34.94 | A |
| ATOM | 148 | CG1 | ILE | A | 167 | −27.780 | 80.613 | −63.835 | 1.00 | 34.93 | A |
| ATOM | 149 | CD1 | ILE | A | 167 | −28.167 | 79.382 | −63.055 | 1.00 | 33.89 | A |
| ATOM | 150 | C | ILE | A | 167 | −29.889 | 83.247 | −65.596 | 1.00 | 35.24 | A |
| ATOM | 151 | O | ILE | A | 167 | −30.280 | 84.178 | −64.901 | 1.00 | 33.88 | A |
| ATOM | 152 | N | LEU | A | 168 | −30.570 | 82.795 | −66.637 | 1.00 | 35.11 | A |
| ATOM | 153 | CA | LEU | A | 168 | −31.877 | 83.314 | −66.987 | 1.00 | 35.36 | A |
| ATOM | 154 | CB | LEU | A | 168 | −31.996 | 83.528 | −68.494 | 1.00 | 34.75 | A |
| ATOM | 155 | CG | LEU | A | 168 | −33.366 | 83.975 | −69.004 | 1.00 | 34.38 | A |
| ATOM | 156 | CD1 | LEU | A | 168 | −33.812 | 85.260 | −68.347 | 1.00 | 32.36 | A |
| ATOM | 157 | CD2 | LEU | A | 168 | −33.271 | 84.153 | −70.500 | 1.00 | 35.39 | A |
| ATOM | 158 | C | LEU | A | 168 | −32.836 | 82.209 | −66.524 | 1.00 | 35.95 | A |
| ATOM | 159 | O | LEU | A | 168 | −32.777 | 81.079 | −67.006 | 1.00 | 35.39 | A |
| ATOM | 160 | N | VAL | A | 169 | −33.701 | 82.541 | −65.573 | 1.00 | 36.20 | A |
| ATOM | 161 | CA | VAL | A | 169 | −34.644 | 81.586 | −65.014 | 1.00 | 35.94 | A |
| ATOM | 162 | CB | VAL | A | 169 | −34.192 | 81.153 | −63.590 | 1.00 | 35.90 | A |
| ATOM | 163 | CG1 | VAL | A | 169 | −32.738 | 80.690 | −63.625 | 1.00 | 34.70 | A |
| ATOM | 164 | CG2 | VAL | A | 169 | −34.341 | 82.321 | −62.594 | 1.00 | 33.08 | A |
| ATOM | 165 | C | VAL | A | 169 | −36.036 | 82.205 | −64.929 | 1.00 | 36.81 | A |
| ATOM | 166 | O | VAL | A | 169 | −36.240 | 83.336 | −65.348 | 1.00 | 36.80 | A |
| ATOM | 167 | N | ARG | A | 170 | −36.989 | 81.452 | −64.391 | 1.00 | 38.62 | A |
| ATOM | 168 | CA | ARG | A | 170 | −38.356 | 81.932 | −64.222 | 1.00 | 41.59 | A |
| ATOM | 169 | CB | ARG | A | 170 | −39.313 | 81.175 | −65.123 | 1.00 | 41.05 | A |
| ATOM | 170 | CG | ARG | A | 170 | −39.163 | 81.373 | −66.607 | 1.00 | 41.93 | A |
| ATOM | 171 | CD | ARG | A | 170 | −40.378 | 80.720 | −67.252 | 1.00 | 41.73 | A |
| ATOM | 172 | NE | ARG | A | 170 | −40.214 | 80.387 | −68.662 | 1.00 | 41.15 | A |
| ATOM | 173 | CZ | ARG | A | 170 | −40.900 | 79.413 | −69.250 | 1.00 | 40.13 | A |
| ATOM | 174 | NH1 | ARG | A | 170 | −41.759 | 78.714 | −68.520 | 1.00 | 38.96 | A |
| ATOM | 175 | NH2 | ARG | A | 170 | −40.753 | 79.153 | −70.546 | 1.00 | 38.13 | A |
| ATOM | 176 | C | ARG | A | 170 | −38.800 | 81.686 | −62.783 | 1.00 | 44.40 | A |
| ATOM | 177 | O | ARG | A | 170 | −38.002 | 81.285 | −61.942 | 1.00 | 46.61 | A |
| ATOM | 178 | N | GLU | A | 171 | −40.077 | 81.928 | −62.506 | 1.00 | 45.48 | A |
| ATOM | 179 | CA | GLU | A | 171 | −40.640 | 81.682 | −61.182 | 1.00 | 46.88 | A |
| ATOM | 180 | CB | GLU | A | 171 | −41.083 | 82.992 | −60.535 | 1.00 | 45.05 | A |
| ATOM | 181 | CG | GLU | A | 171 | −41.852 | 82.821 | −59.234 | 1.00 | 44.74 | A |
| ATOM | 182 | CD | GLU | A | 171 | −41.591 | 83.945 | −58.253 | 1.00 | 43.50 | A |
| ATOM | 183 | OE1 | GLU | A | 171 | −41.502 | 85.109 | −58.697 | 1.00 | 42.40 | A |
| ATOM | 184 | OE2 | GLU | A | 171 | −41.480 | 83.663 | −57.040 | 1.00 | 41.55 | A |
| ATOM | 185 | C | GLU | A | 171 | −41.829 | 80.754 | −61.416 | 1.00 | 48.84 | A |
| ATOM | 186 | O | GLU | A | 171 | −42.913 | 81.204 | −61.766 | 1.00 | 50.30 | A |
| ATOM | 187 | N | LYS | A | 172 | −41.606 | 79.457 | −61.233 | 1.00 | 50.01 | A |
| ATOM | 188 | CA | LYS | A | 172 | −42.616 | 78.423 | −61.470 | 1.00 | 52.21 | A |
| ATOM | 189 | CB | LYS | A | 172 | −42.217 | 77.130 | −60.747 | 1.00 | 52.80 | A |
| ATOM | 190 | CG | LYS | A | 172 | −41.128 | 76.319 | −61.447 | 1.00 | 52.82 | A |
| ATOM | 191 | CD | LYS | A | 172 | −40.664 | 75.147 | −60.592 | 1.00 | 52.49 | A |
| ATOM | 192 | CE | LYS | A | 172 | −39.542 | 74.371 | −61.272 | 1.00 | 53.23 | A |
| ATOM | 193 | NZ | LYS | A | 172 | −39.027 | 73.244 | −60.432 | 1.00 | 54.40 | A |
| ATOM | 194 | C | LYS | A | 172 | −44.087 | 78.707 | −61.177 | 1.00 | 53.85 | A |
| ATOM | 195 | O | LYS | A | 172 | −44.947 | 77.914 | −61.566 | 1.00 | 54.79 | A |
| ATOM | 196 | N | ALA | A | 173 | −44.392 | 79.816 | −60.510 | 1.00 | 54.56 | A |
| ATOM | 197 | CA | ALA | A | 173 | −45.782 | 80.127 | −60.183 | 1.00 | 54.27 | A |
| ATOM | 198 | CB | ALA | A | 173 | −45.997 | 80.000 | −58.678 | 1.00 | 53.96 | A |
| ATOM | 199 | C | ALA | A | 173 | −46.231 | 81.504 | −60.655 | 1.00 | 54.12 | A |
| ATOM | 200 | O | ALA | A | 173 | −47.410 | 81.836 | −60.576 | 1.00 | 53.43 | A |
| ATOM | 201 | N | THR | A | 174 | −45.292 | 82.295 | −61.161 | 1.00 | 53.53 | A |
| ATOM | 202 | CA | THR | A | 174 | −45.604 | 83.640 | −61.617 | 1.00 | 51.68 | A |
| ATOM | 203 | CB | THR | A | 174 | −44.958 | 84.692 | −60.695 | 1.00 | 50.61 | A |
| ATOM | 204 | OG1 | THR | A | 174 | −43.533 | 84.639 | −60.833 | 0.00 | 51.12 | A |
| ATOM | 205 | CG2 | THR | A | 174 | −45.331 | 84.428 | −59.243 | 0.00 | 51.12 | A |
| ATOM | 206 | C | THR | A | 174 | −45.162 | 83.925 | −63.048 | 1.00 | 51.48 | A |
| ATOM | 207 | O | THR | A | 174 | −45.578 | 84.918 | −63.630 | 1.00 | 52.45 | A |
| ATOM | 208 | N | GLY | A | 175 | −44.318 | 83.070 | −63.618 | 1.00 | 50.81 | A |
| ATOM | 209 | CA | GLY | A | 175 | −43.863 | 83.300 | −64.981 | 1.00 | 50.84 | A |
| ATOM | 210 | C | GLY | A | 175 | −43.141 | 84.624 | −65.200 | 1.00 | 51.27 | A |
| ATOM | 211 | O | GLY | A | 175 | −42.827 | 84.990 | −66.327 | 1.00 | 51.55 | A |
| ATOM | 212 | N | ARG | A | 176 | −42.883 | 85.350 | −64.121 | 1.00 | 51.58 | A |
| ATOM | 213 | CA | ARG | A | 176 | −42.181 | 86.624 | −64.189 | 1.00 | 52.10 | A |
| ATOM | 214 | CB | ARG | A | 176 | −42.433 | 87.390 | −62.882 | 1.00 | 52.63 | A |
| ATOM | 215 | CG | ARG | A | 176 | −41.724 | 88.736 | −62.732 | 1.00 | 52.82 | A |
| ATOM | 216 | CD | ARG | A | 176 | −42.157 | 89.432 | −61.428 | 1.00 | 52.17 | A |
| ATOM | 217 | NE | ARG | A | 176 | −43.182 | 90.451 | −61.659 | 1.00 | 51.02 | A |
| ATOM | 218 | CZ | ARG | A | 176 | −43.923 | 91.009 | −60.703 | 1.00 | 50.91 | A |
| ATOM | 219 | NH1 | ARG | A | 176 | −43.770 | 90.648 | −59.436 | 1.00 | 50.44 | A |
| ATOM | 220 | NH2 | ARG | A | 176 | −44.813 | 91.940 | −61.016 | 1.00 | 48.56 | A |
| ATOM | 221 | C | ARG | A | 176 | −40.688 | 86.310 | −64.384 | 1.00 | 52.83 | A |
| ATOM | 222 | O | ARG | A | 176 | −40.210 | 85.275 | −63.913 | 1.00 | 53.10 | A |
| ATOM | 223 | N | TYR | A | 177 | −39.955 | 87.188 | −65.077 | 1.00 | 52.76 | A |
| ATOM | 224 | CA | TYR | A | 177 | −38.527 | 86.956 | −65.339 | 1.00 | 50.54 | A |
| ATOM | 225 | CB | TYR | A | 177 | −38.167 | 87.324 | −66.772 | 1.00 | 48.93 | A |
| ATOM | 226 | CG | TYR | A | 177 | −38.699 | 86.366 | −67.789 | 1.00 | 48.99 | A |
| ATOM | 227 | CD1 | TYR | A | 177 | −39.671 | 86.768 | −68.689 | 1.00 | 49.82 | A |
| ATOM | 228 | CE1 | TYR | A | 177 | −40.198 | 85.893 | −69.622 | 1.00 | 49.26 | A |
| ATOM | 229 | CD2 | TYR | A | 177 | −38.250 | 85.050 | −67.845 | 1.00 | 49.34 | A |
| ATOM | 230 | CE2 | TYR | A | 177 | −38.768 | 84.161 | −68.779 | 1.00 | 47.95 | A |
| ATOM | 231 | CZ | TYR | A | 177 | −39.746 | 84.599 | −69.661 | 1.00 | 47.44 | A |
| ATOM | 232 | OH | TYR | A | 177 | −40.294 | 83.757 | −70.583 | 1.00 | 46.98 | A |
| ATOM | 233 | C | TYR | A | 177 | −37.515 | 87.632 | −64.433 | 1.00 | 49.87 | A |
| ATOM | 234 | O | TYR | A | 177 | −37.745 | 88.714 | −63.899 | 1.00 | 49.64 | A |
| ATOM | 235 | N | TYR | A | 178 | −36.376 | 86.963 | −64.288 | 1.00 | 49.12 | A |
| ATOM | 236 | CA | TYR | A | 178 | −35.271 | 87.436 | −63.473 | 1.00 | 47.33 | A |
| ATOM | 237 | CB | TYR | A | 178 | −35.451 | 87.049 | −62.003 | 1.00 | 49.56 | A |
| ATOM | 238 | CG | TYR | A | 178 | −36.827 | 87.256 | −61.432 | 1.00 | 51.23 | A |
| ATOM | 239 | CD1 | TYR | A | 178 | −37.860 | 86.370 | −61.723 | 1.00 | 53.31 | A |
| ATOM | 240 | CE1 | TYR | A | 178 | −39.120 | 86.545 | −61.190 | 1.00 | 53.33 | A |
| ATOM | 241 | CD2 | TYR | A | 178 | −37.096 | 88.323 | −60.594 | 1.00 | 50.67 | A |
| ATOM | 242 | CE2 | TYR | A | 178 | −38.347 | 88.504 | −60.059 | 1.00 | 51.62 | A |
| ATOM | 243 | CZ | TYR | A | 178 | −39.357 | 87.612 | −60.353 | 1.00 | 52.88 | A |
| ATOM | 244 | OH | TYR | A | 178 | −40.599 | 87.757 | −59.772 | 1.00 | 55.62 | A |
| ATOM | 245 | C | TYR | A | 178 | −33.980 | 86.796 | −63.960 | 1.00 | 45.27 | A |
| ATOM | 246 | O | TYR | A | 178 | −33.989 | 85.747 | −64.602 | 1.00 | 42.18 | A |
| ATOM | 247 | N | ALA | A | 179 | −32.872 | 87.446 | −63.630 | 1.00 | 43.82 | A |
| ATOM | 248 | CA | ALA | A | 179 | −31.547 | 86.975 | −63.984 | 1.00 | 43.28 | A |
| ATOM | 249 | CB | ALA | A | 179 | −30.752 | 88.104 | −64.626 | 1.00 | 43.85 | A |
| ATOM | 250 | C | ALA | A | 179 | −30.914 | 86.558 | −62.667 | 1.00 | 42.16 | A |
| ATOM | 251 | O | ALA | A | 179 | −30.592 | 87.410 | −61.850 | 1.00 | 43.31 | A |
| ATOM | 252 | N | MET | A | 180 | −30.751 | 85.253 | −62.458 | 1.00 | 40.86 | A |
| ATOM | 253 | CA | MET | A | 180 | −30.185 | 84.733 | −61.211 | 1.00 | 40.26 | A |
| ATOM | 254 | CB | MET | A | 180 | −30.832 | 83.391 | −60.863 | 1.00 | 39.84 | A |
| ATOM | 255 | CG | MET | A | 180 | −29.922 | 82.436 | −60.109 | 1.00 | 38.62 | A |
| ATOM | 256 | SD | MET | A | 180 | −30.775 | 81.338 | −58.971 | 1.00 | 38.70 | A |
| ATOM | 257 | CE | MET | A | 180 | −31.920 | 80.493 | −60.037 | 1.00 | 35.83 | A |
| ATOM | 258 | C | MET | A | 180 | −28.668 | 84.591 | −61.145 | 1.00 | 39.94 | A |
| ATOM | 259 | O | MET | A | 180 | −28.055 | 83.835 | −61.899 | 1.00 | 39.52 | A |
| ATOM | 260 | N | LYS | A | 181 | −28.071 | 85.315 | −60.209 | 1.00 | 38.57 | A |
| ATOM | 261 | CA | LYS | A | 181 | −26.631 | 85.285 | −60.034 | 1.00 | 38.78 | A |
| ATOM | 262 | CB | LYS | A | 181 | −26.117 | 86.667 | −59.612 | 1.00 | 41.30 | A |
| ATOM | 263 | CG | LYS | A | 181 | −24.597 | 86.768 | −59.501 | 1.00 | 41.79 | A |
| ATOM | 264 | CD | LYS | A | 181 | −24.173 | 87.958 | −58.652 | 1.00 | 42.77 | A |
| ATOM | 265 | CE | LYS | A | 181 | −24.683 | 89.247 | −59.228 | 1.00 | 44.50 | A |
| ATOM | 266 | NZ | LYS | A | 181 | −24.047 | 89.504 | −60.543 | 1.00 | 46.45 | A |
| ATOM | 267 | C | LYS | A | 181 | −26.259 | 84.264 | −58.977 | 1.00 | 37.44 | A |
| ATOM | 268 | O | LYS | A | 181 | −26.646 | 84.390 | −57.825 | 1.00 | 36.11 | A |
| ATOM | 269 | N | ILE | A | 182 | −25.506 | 83.252 | −59.383 | 1.00 | 36.93 | A |
| ATOM | 270 | CA | ILE | A | 182 | −25.071 | 82.214 | −58.462 | 1.00 | 37.57 | A |
| ATOM | 271 | CB | ILE | A | 182 | −25.491 | 80.787 | −58.939 | 1.00 | 36.60 | A |
| ATOM | 272 | CG2 | ILE | A | 182 | −25.537 | 79.838 | −57.756 | 1.00 | 33.85 | A |
| ATOM | 273 | CG1 | ILE | A | 182 | −26.874 | 80.826 | −59.595 | 1.00 | 36.23 | A |
| ATOM | 274 | CD1 | ILE | A | 182 | −27.435 | 79.471 | −59.945 | 1.00 | 34.13 | A |
| ATOM | 275 | C | ILE | A | 182 | −23.551 | 82.263 | −58.353 | 1.00 | 38.41 | A |
| ATOM | 276 | O | ILE | A | 182 | −22.841 | 81.945 | −59.297 | 1.00 | 38.20 | A |
| ATOM | 277 | N | LEU | A | 183 | −23.059 | 82.696 | −57.204 | 1.00 | 39.83 | A |
| ATOM | 278 | CA | LEU | A | 183 | −21.629 | 82.762 | −56.976 | 1.00 | 42.27 | A |
| ATOM | 279 | CB | LEU | A | 183 | −21.254 | 84.014 | −56.186 | 1.00 | 40.30 | A |
| ATOM | 280 | CG | LEU | A | 183 | −22.203 | 85.202 | −56.116 | 1.00 | 37.57 | A |
| ATOM | 281 | CD1 | LEU | A | 183 | −21.705 | 86.182 | −55.077 | 1.00 | 35.14 | A |
| ATOM | 282 | CD2 | LEU | A | 183 | −22.295 | 85.847 | −57.465 | 1.00 | 36.20 | A |
| ATOM | 283 | C | LEU | A | 183 | −21.366 | 81.555 | −56.102 | 1.00 | 45.16 | A |
| ATOM | 284 | O | LEU | A | 183 | −22.288 | 81.054 | −55.468 | 1.00 | 45.96 | A |
| ATOM | 285 | N | ARG | A | 184 | −20.120 | 81.098 | −56.050 | 1.00 | 48.36 | A |
| ATOM | 286 | CA | ARG | A | 184 | −19.781 | 79.949 | −55.218 | 1.00 | 51.03 | A |
| ATOM | 287 | CB | ARG | A | 184 | −18.692 | 79.116 | −55.899 | 1.00 | 51.26 | A |
| ATOM | 288 | CG | ARG | A | 184 | −19.112 | 78.584 | −57.249 | 1.00 | 52.11 | A |
| ATOM | 289 | CD | ARG | A | 184 | −17.927 | 78.342 | −58.170 | 1.00 | 53.15 | A |
| ATOM | 290 | NE | ARG | A | 184 | −18.382 | 78.278 | −59.562 | 1.00 | 54.88 | A |
| ATOM | 291 | CZ | ARG | A | 184 | −18.953 | 77.210 | −60.127 | 1.00 | 54.67 | A |
| ATOM | 292 | NH1 | ARG | A | 184 | −19.133 | 76.093 | −59.423 | 1.00 | 53.05 | A |
| ATOM | 293 | NH2 | ARG | A | 184 | −19.364 | 77.269 | −61.393 | 1.00 | 53.05 | A |
| ATOM | 294 | C | ARG | A | 184 | −19.326 | 80.370 | −53.815 | 1.00 | 51.71 | A |
| ATOM | 295 | O | ARG | A | 184 | −18.136 | 80.490 | −53.544 | 1.00 | 51.78 | A |
| ATOM | 296 | N | LYS | A | 185 | −20.291 | 80.612 | −52.934 | 1.00 | 53.52 | A |
| ATOM | 297 | CA | LYS | A | 185 | −20.010 | 80.998 | −51.553 | 1.00 | 55.78 | A |
| ATOM | 298 | CB | LYS | A | 185 | −21.175 | 80.593 | −50.651 | 1.00 | 54.52 | A |
| ATOM | 299 | CG | LYS | A | 185 | −20.866 | 80.567 | −49.166 | 1.00 | 51.97 | A |
| ATOM | 300 | CD | LYS | A | 185 | −21.797 | 79.603 | −48.424 | 1.00 | 50.42 | A |
| ATOM | 301 | CE | LYS | A | 185 | −23.268 | 79.917 | −48.670 | 1.00 | 48.41 | A |
| ATOM | 302 | NZ | LYS | A | 185 | −24.191 | 79.115 | −47.811 | 1.00 | 45.97 | A |
| ATOM | 303 | C | LYS | A | 185 | −18.776 | 80.238 | −51.131 | 1.00 | 57.59 | A |
| ATOM | 304 | O | LYS | A | 185 | −17.785 | 80.820 | −50.685 | 1.00 | 57.98 | A |
| ATOM | 305 | N | GLU | A | 186 | −18.869 | 78.922 | −51.302 | 1.00 | 59.79 | A |
| ATOM | 306 | CA | GLU | A | 186 | −17.813 | 77.971 | −50.972 | 1.00 | 60.74 | A |
| ATOM | 307 | CB | GLU | A | 186 | −18.065 | 76.650 | −51.705 | 1.00 | 58.76 | A |
| ATOM | 308 | CG | GLU | A | 186 | −17.102 | 75.543 | −51.345 | 1.00 | 58.03 | A |
| ATOM | 309 | CD | GLU | A | 186 | −17.312 | 75.026 | −49.939 | 0.00 | 57.11 | A |
| ATOM | 310 | OE1 | GLU | A | 186 | −16.537 | 74.150 | −49.501 | 0.00 | 57.26 | A |
| ATOM | 311 | OE2 | GLU | A | 186 | −18.257 | 75.494 | −49.273 | 1.00 | 55.43 | A |
| ATOM | 312 | C | GLU | A | 186 | −16.431 | 78.495 | −51.344 | 1.00 | 62.32 | A |
| ATOM | 313 | O | GLU | A | 186 | −15.778 | 79.193 | −50.560 | 1.00 | 61.63 | A |
| ATOM | 314 | N | VAL | A | 187 | −15.992 | 78.163 | −52.551 | 1.00 | 63.85 | A |
| ATOM | 315 | CA | VAL | A | 187 | −14.678 | 78.587 | −52.992 | 1.00 | 64.76 | A |
| ATOM | 316 | CB | VAL | A | 187 | −14.340 | 78.038 | −54.416 | 1.00 | 65.26 | A |
| ATOM | 317 | CG1 | VAL | A | 187 | −14.061 | 76.522 | −54.325 | 1.00 | 63.82 | A |
| ATOM | 318 | CG2 | VAL | A | 187 | −15.482 | 78.315 | −55.395 | 1.00 | 64.58 | A |
| ATOM | 319 | C | VAL | A | 187 | −14.471 | 80.091 | −52.932 | 1.00 | 65.28 | A |
| ATOM | 320 | O | VAL | A | 187 | −13.347 | 80.546 | −53.070 | 1.00 | 65.80 | A |
| ATOM | 321 | N | ILE | A | 188 | −15.534 | 80.861 | −52.703 | 1.00 | 65.89 | A |
| ATOM | 322 | CA | ILE | A | 188 | −15.391 | 82.318 | −52.598 | 1.00 | 66.91 | A |
| ATOM | 323 | CB | ILE | A | 188 | −16.698 | 83.064 | −52.972 | 1.00 | 67.17 | A |
| ATOM | 324 | CG2 | ILE | A | 188 | −16.913 | 84.267 | −52.049 | 1.00 | 66.41 | A |
| ATOM | 325 | CG1 | ILE | A | 188 | −16.628 | 83.512 | −54.441 | 1.00 | 67.59 | A |
| ATOM | 326 | CD1 | ILE | A | 188 | −15.415 | 84.421 | −54.792 | 1.00 | 65.98 | A |
| ATOM | 327 | C | ILE | A | 188 | −14.932 | 82.764 | −51.202 | 1.00 | 67.30 | A |
| ATOM | 328 | O | ILE | A | 188 | −14.443 | 83.889 | −51.033 | 1.00 | 67.01 | A |
| ATOM | 329 | N | ILE | A | 189 | −15.104 | 81.886 | −50.210 | 1.00 | 66.94 | A |
| ATOM | 330 | CA | ILE | A | 189 | −14.660 | 82.166 | −48.846 | 1.00 | 65.50 | A |
| ATOM | 331 | CB | ILE | A | 189 | −15.014 | 81.031 | −47.868 | 1.00 | 63.89 | A |
| ATOM | 332 | CG2 | ILE | A | 189 | −14.313 | 81.254 | −46.547 | 1.00 | 62.84 | A |
| ATOM | 333 | CG1 | ILE | A | 189 | −16.520 | 80.936 | −47.666 | 1.00 | 63.52 | A |
| ATOM | 334 | CD1 | ILE | A | 189 | −16.924 | 79.812 | −46.705 | 1.00 | 62.06 | A |
| ATOM | 335 | C | ILE | A | 189 | −13.147 | 82.178 | −48.942 | 1.00 | 66.75 | A |
| ATOM | 336 | O | ILE | A | 189 | −12.465 | 82.909 | −48.222 | 1.00 | 66.52 | A |
| ATOM | 337 | N | ALA | A | 190 | −12.645 | 81.345 | −49.855 | 1.00 | 68.03 | A |
| ATOM | 338 | CA | ALA | A | 190 | −11.214 | 81.183 | −50.089 | 1.00 | 69.25 | A |
| ATOM | 339 | CB | ALA | A | 190 | −10.869 | 79.696 | −50.108 | 1.00 | 69.48 | A |
| ATOM | 340 | C | ALA | A | 190 | −10.720 | 81.847 | −51.375 | 1.00 | 69.13 | A |
| ATOM | 341 | O | ALA | A | 190 | −9.862 | 81.297 | −52.075 | 1.00 | 68.80 | A |
| ATOM | 342 | N | LYS | A | 191 | −11.257 | 83.023 | −51.684 | 1.00 | 68.08 | A |
| ATOM | 343 | CA | LYS | A | 191 | −10.851 | 83.747 | −52.881 | 1.00 | 68.08 | A |
| ATOM | 344 | CB | LYS | A | 191 | −11.950 | 83.681 | −53.945 | 1.00 | 67.83 | A |
| ATOM | 345 | CG | LYS | A | 191 | −11.939 | 82.365 | −54.717 | 1.00 | 66.64 | A |
| ATOM | 346 | CD | LYS | A | 191 | −13.278 | 82.051 | −55.359 | 1.00 | 64.94 | A |
| ATOM | 347 | CE | LYS | A | 191 | −13.264 | 80.658 | −55.981 | 1.00 | 63.39 | A |
| ATOM | 348 | NZ | LYS | A | 191 | −14.459 | 80.412 | −56.855 | 1.00 | 62.03 | A |
| ATOM | 349 | C | LYS | A | 191 | −10.562 | 85.172 | −52.480 | 1.00 | 67.99 | A |
| ATOM | 350 | O | LYS | A | 191 | −10.608 | 86.105 | −53.287 | 1.00 | 68.39 | A |
| ATOM | 351 | N | ASP | A | 192 | −10.271 | 85.316 | −51.196 | 1.00 | 68.39 | A |
| ATOM | 352 | CA | ASP | A | 192 | −9.939 | 86.593 | −50.596 | 1.00 | 68.75 | A |
| ATOM | 353 | CB | ASP | A | 192 | −8.516 | 86.968 | −50.983 | 1.00 | 69.38 | A |
| ATOM | 354 | CG | ASP | A | 192 | −7.527 | 85.928 | −50.533 | 1.00 | 69.87 | A |
| ATOM | 355 | OD1 | ASP | A | 192 | −7.744 | 85.385 | −49.420 | 1.00 | 69.31 | A |
| ATOM | 356 | OD2 | ASP | A | 192 | −6.552 | 85.657 | −51.274 | 1.00 | 70.22 | A |
| ATOM | 357 | C | ASP | A | 192 | −10.869 | 87.763 | −50.844 | 1.00 | 67.98 | A |
| ATOM | 358 | O | ASP | A | 192 | −10.509 | 88.900 | −50.553 | 1.00 | 69.16 | A |
| ATOM | 359 | N | GLU | A | 193 | −12.059 | 87.504 | −51.372 | 1.00 | 66.63 | A |
| ATOM | 360 | CA | GLU | A | 193 | −13.007 | 88.585 | −51.593 | 1.00 | 65.37 | A |
| ATOM | 361 | CB | GLU | A | 193 | −13.360 | 88.726 | −53.074 | 1.00 | 62.79 | A |
| ATOM | 362 | CG | GLU | A | 193 | −12.398 | 89.623 | −53.832 | 1.00 | 59.71 | A |
| ATOM | 363 | CD | GLU | A | 193 | −13.106 | 90.528 | −54.823 | 1.00 | 58.50 | A |
| ATOM | 364 | OE1 | GLU | A | 193 | −12.811 | 90.413 | −56.036 | 1.00 | 57.60 | A |
| ATOM | 365 | OE2 | GLU | A | 193 | −13.951 | 91.354 | −54.383 | 1.00 | 54.02 | A |
| ATOM | 366 | C | GLU | A | 193 | −14.267 | 88.396 | −50.763 | 1.00 | 65.86 | A |
| ATOM | 367 | O | GLU | A | 193 | −15.384 | 88.491 | −51.264 | 1.00 | 65.15 | A |
| ATOM | 368 | N | VAL | A | 194 | −14.071 | 88.129 | −49.477 | 1.00 | 66.94 | A |
| ATOM | 369 | CA | VAL | A | 194 | −15.191 | 87.950 | −48.570 | 1.00 | 67.58 | A |
| ATOM | 370 | CB | VAL | A | 194 | −14.710 | 87.716 | −47.105 | 1.00 | 67.57 | A |
| ATOM | 371 | CG1 | VAL | A | 194 | −15.321 | 86.417 | −46.551 | 1.00 | 65.78 | A |
| ATOM | 372 | CG2 | VAL | A | 194 | −13.184 | 87.664 | −47.053 | 1.00 | 66.34 | A |
| ATOM | 373 | C | VAL | A | 194 | −16.091 | 89.190 | −48.618 | 1.00 | 68.02 | A |
| ATOM | 374 | O | VAL | A | 194 | −16.796 | 89.404 | −49.602 | 1.00 | 67.33 | A |
| ATOM | 375 | N | ALA | A | 195 | −16.041 | 90.010 | −47.570 | 1.00 | 68.20 | A |
| ATOM | 376 | CA | ALA | A | 195 | −16.872 | 91.216 | −47.467 | 1.00 | 69.06 | A |
| ATOM | 377 | CB | ALA | A | 195 | −16.359 | 92.100 | −46.311 | 1.00 | 69.49 | A |
| ATOM | 378 | C | ALA | A | 195 | −17.057 | 92.071 | −48.734 | 1.00 | 68.86 | A |
| ATOM | 379 | O | ALA | A | 195 | −18.164 | 92.554 | −49.002 | 1.00 | 68.15 | A |
| ATOM | 380 | N | HIS | A | 196 | −15.987 | 92.276 | −49.500 | 1.00 | 68.70 | A |
| ATOM | 381 | CA | HIS | A | 196 | −16.071 | 93.083 | −50.719 | 1.00 | 69.07 | A |
| ATOM | 382 | CB | HIS | A | 196 | −14.693 | 93.198 | −51.371 | 1.00 | 71.05 | A |
| ATOM | 383 | CG | HIS | A | 196 | −13.725 | 94.056 | −50.615 | 1.00 | 72.47 | A |
| ATOM | 384 | CD2 | HIS | A | 196 | −12.651 | 93.731 | −49.856 | 1.00 | 72.84 | A |
| ATOM | 385 | ND1 | HIS | A | 196 | −13.798 | 95.432 | −50.607 | 1.00 | 72.65 | A |
| ATOM | 386 | CE1 | HIS | A | 196 | −12.810 | 95.918 | −49.877 | 1.00 | 72.90 | A |
| ATOM | 387 | NE2 | HIS | A | 196 | −12.099 | 94.907 | −49.410 | 1.00 | 73.11 | A |
| ATOM | 388 | C | HIS | A | 196 | −17.039 | 92.460 | −51.721 | 1.00 | 68.13 | A |
| ATOM | 389 | O | HIS | A | 196 | −17.380 | 93.071 | −52.734 | 1.00 | 66.34 | A |
| ATOM | 390 | N | THR | A | 197 | −17.456 | 91.230 | −51.426 | 1.00 | 67.28 | A |
| ATOM | 391 | CA | THR | A | 197 | −18.374 | 90.467 | −52.265 | 1.00 | 66.08 | A |
| ATOM | 392 | CB | THR | A | 197 | −17.713 | 89.150 | −52.738 | 1.00 | 66.62 | A |
| ATOM | 393 | OG1 | THR | A | 197 | −16.453 | 89.451 | −53.346 | 1.00 | 67.62 | A |
| ATOM | 394 | CG2 | THR | A | 197 | −18.574 | 88.437 | −53.761 | 1.00 | 67.49 | A |
| ATOM | 395 | C | THR | A | 197 | −19.622 | 90.161 | −51.445 | 1.00 | 65.19 | A |
| ATOM | 396 | O | THR | A | 197 | −20.541 | 89.482 | −51.899 | 1.00 | 63.58 | A |
| ATOM | 397 | N | VAL | A | 198 | −19.627 | 90.659 | −50.214 | 1.00 | 65.19 | A |
| ATOM | 398 | CA | VAL | A | 198 | −20.763 | 90.499 | −49.319 | 1.00 | 64.81 | A |
| ATOM | 399 | CB | VAL | A | 198 | −20.336 | 90.441 | −47.835 | 1.00 | 63.54 | A |
| ATOM | 400 | CG1 | VAL | A | 198 | −21.557 | 90.500 | −46.952 | 1.00 | 62.91 | A |
| ATOM | 401 | CG2 | VAL | A | 198 | −19.575 | 89.163 | −47.548 | 1.00 | 62.42 | A |
| ATOM | 402 | C | VAL | A | 198 | −21.571 | 91.768 | −49.540 | 1.00 | 66.34 | A |
| ATOM | 403 | O | VAL | A | 198 | −22.708 | 91.721 | −50.017 | 1.00 | 66.25 | A |
| ATOM | 404 | N | THR | A | 199 | −20.952 | 92.902 | −49.213 | 1.00 | 66.72 | A |
| ATOM | 405 | CA | THR | A | 199 | −21.572 | 94.216 | −49.367 | 1.00 | 66.80 | A |
| ATOM | 406 | CB | THR | A | 199 | −20.503 | 95.310 | −49.471 | 1.00 | 66.00 | A |
| ATOM | 407 | OG1 | THR | A | 199 | −19.378 | 94.957 | −48.654 | 1.00 | 63.44 | A |
| ATOM | 408 | CG2 | THR | A | 199 | −21.067 | 96.632 | −48.986 | 1.00 | 65.21 | A |
| ATOM | 409 | C | THR | A | 199 | −22.445 | 94.240 | −50.620 | 1.00 | 67.92 | A |
| ATOM | 410 | O | THR | A | 199 | −23.625 | 94.612 | −50.567 | 1.00 | 67.59 | A |
| ATOM | 411 | N | GLU | A | 200 | −21.856 | 93.833 | −51.743 | 1.00 | 68.85 | A |
| ATOM | 412 | CA | GLU | A | 200 | −22.571 | 93.756 | −53.017 | 1.00 | 69.27 | A |
| ATOM | 413 | CB | GLU | A | 200 | −21.880 | 92.753 | −53.960 | 1.00 | 69.46 | A |
| ATOM | 414 | CG | GLU | A | 200 | −22.757 | 92.311 | −55.133 | 1.00 | 69.97 | A |
| ATOM | 415 | CD | GLU | A | 200 | −22.830 | 90.792 | −55.307 | 1.00 | 70.34 | A |
| ATOM | 416 | OE1 | GLU | A | 200 | −23.198 | 90.079 | −54.345 | 1.00 | 69.12 | A |
| ATOM | 417 | OE2 | GLU | A | 200 | −22.532 | 90.314 | −56.423 | 1.00 | 71.08 | A |
| ATOM | 418 | C | GLU | A | 200 | −23.998 | 93.282 | −52.751 | 1.00 | 68.62 | A |
| ATOM | 419 | O | GLU | A | 200 | −24.949 | 93.756 | −53.365 | 1.00 | 67.85 | A |
| ATOM | 420 | N | SER | A | 201 | −24.125 | 92.337 | −51.826 | 1.00 | 68.47 | A |
| ATOM | 421 | CA | SER | A | 201 | −25.416 | 91.773 | −51.467 | 1.00 | 69.83 | A |
| ATOM | 422 | CB | SER | A | 201 | −25.237 | 90.415 | −50.799 | 1.00 | 69.46 | A |
| ATOM | 423 | OG | SER | A | 201 | −26.427 | 90.062 | −50.105 | 1.00 | 69.45 | A |
| ATOM | 424 | C | SER | A | 201 | −26.235 | 92.635 | −50.527 | 1.00 | 70.17 | A |
| ATOM | 425 | O | SER | A | 201 | −27.455 | 92.759 | −50.688 | 1.00 | 71.51 | A |
| ATOM | 426 | N | ARG | A | 202 | −25.562 | 93.199 | −49.527 | 1.00 | 69.38 | A |
| ATOM | 427 | CA | ARG | A | 202 | −26.223 | 94.021 | −48.528 | 1.00 | 68.27 | A |
| ATOM | 428 | CB | ARG | A | 202 | −25.198 | 94.516 | −47.504 | 1.00 | 69.10 | A |
| ATOM | 429 | CG | ARG | A | 202 | −24.504 | 93.396 | −46.732 | 1.00 | 69.21 | A |
| ATOM | 430 | CD | ARG | A | 202 | −25.412 | 92.780 | −45.679 | 1.00 | 69.20 | A |
| ATOM | 431 | NE | ARG | A | 202 | −24.941 | 91.464 | −45.254 | 1.00 | 70.05 | A |
| ATOM | 432 | CZ | ARG | A | 202 | −23.829 | 91.235 | −44.562 | 1.00 | 70.53 | A |
| ATOM | 433 | NH1 | ARG | A | 202 | −23.049 | 92.242 | −44.195 | 1.00 | 71.39 | A |
| ATOM | 434 | NH2 | ARG | A | 202 | −23.487 | 89.990 | −44.247 | 1.00 | 70.02 | A |
| ATOM | 435 | C | ARG | A | 202 | −26.947 | 95.203 | −49.159 | 1.00 | 67.39 | A |
| ATOM | 436 | O | ARG | A | 202 | −28.042 | 95.583 | −48.731 | 1.00 | 66.26 | A |
| ATOM | 437 | N | VAL | A | 203 | −26.363 | 95.770 | −50.202 | 1.00 | 66.06 | A |
| ATOM | 438 | CA | VAL | A | 203 | −27.003 | 96.922 | −50.806 | 1.00 | 65.47 | A |
| ATOM | 439 | CB | VAL | A | 203 | −26.018 | 98.085 | −50.835 | 1.00 | 63.69 | A |
| ATOM | 440 | CG1 | VAL | A | 203 | −26.775 | 99.408 | −51.003 | 1.00 | 62.35 | A |
| ATOM | 441 | CG2 | VAL | A | 203 | −25.232 | 98.071 | −49.536 | 1.00 | 61.10 | A |
| ATOM | 442 | C | VAL | A | 203 | −27.669 | 96.702 | −52.174 | 1.00 | 65.54 | A |
| ATOM | 443 | O | VAL | A | 203 | −28.628 | 97.400 | −52.508 | 1.00 | 66.36 | A |
| ATOM | 444 | N | LEU | A | 204 | −27.184 | 95.745 | −52.958 | 1.00 | 64.28 | A |
| ATOM | 445 | CA | LEU | A | 204 | −27.826 | 95.461 | −54.231 | 1.00 | 63.96 | A |
| ATOM | 446 | CB | LEU | A | 204 | −27.281 | 94.146 | −54.815 | 1.00 | 66.28 | A |
| ATOM | 447 | CG | LEU | A | 204 | −28.031 | 93.292 | −55.859 | 1.00 | 67.36 | A |
| ATOM | 448 | CD1 | LEU | A | 204 | −28.358 | 94.077 | −57.133 | 1.00 | 66.35 | A |
| ATOM | 449 | CD2 | LEU | A | 204 | −27.159 | 92.087 | −56.188 | 1.00 | 67.07 | A |
| ATOM | 450 | C | LEU | A | 204 | −29.328 | 95.341 | −53.949 | 1.00 | 63.04 | A |
| ATOM | 451 | O | LEU | A | 204 | −30.157 | 95.596 | −54.825 | 1.00 | 62.93 | A |
| ATOM | 452 | N | GLN | A | 205 | −29.670 | 94.973 | −52.713 | 1.00 | 62.12 | A |
| ATOM | 453 | CA | GLN | A | 205 | −31.075 | 94.823 | −52.312 | 1.00 | 60.78 | A |
| ATOM | 454 | CB | GLN | A | 205 | −31.217 | 93.700 | −51.268 | 1.00 | 61.18 | A |
| ATOM | 455 | CG | GLN | A | 205 | −31.141 | 94.128 | −49.805 | 1.00 | 61.58 | A |
| ATOM | 456 | CD | GLN | A | 205 | −32.497 | 94.033 | −49.100 | 1.00 | 61.72 | A |
| ATOM | 457 | OE1 | GLN | A | 205 | −32.625 | 94.380 | −47.917 | 1.00 | 60.53 | A |
| ATOM | 458 | NE2 | GLN | A | 205 | −33.514 | 93.561 | −49.826 | 1.00 | 59.94 | A |
| ATOM | 459 | C | GLN | A | 205 | −31.710 | 96.121 | −51.786 | 1.00 | 59.08 | A |
| ATOM | 460 | O | GLN | A | 205 | −32.916 | 96.298 | −51.884 | 1.00 | 58.87 | A |
| ATOM | 461 | N | ASN | A | 206 | −30.892 | 97.013 | −51.231 | 1.00 | 57.04 | A |
| ATOM | 462 | CA | ASN | A | 206 | −31.356 | 98.303 | −50.714 | 1.00 | 55.33 | A |
| ATOM | 463 | CB | ASN | A | 206 | −30.452 | 98.773 | −49.574 | 1.00 | 55.76 | A |
| ATOM | 464 | CG | ASN | A | 206 | −30.886 | 98.252 | −48.221 | 1.00 | 54.49 | A |
| ATOM | 465 | OD1 | ASN | A | 206 | −31.579 | 98.941 | −47.460 | 1.00 | 52.73 | A |
| ATOM | 466 | ND2 | ASN | A | 206 | −30.479 | 97.031 | −47.911 | 1.00 | 54.14 | A |
| ATOM | 467 | C | ASN | A | 206 | −31.272 | 99.318 | −51.852 | 1.00 | 53.75 | A |
| ATOM | 468 | O | ASN | A | 206 | −32.126 | 100.202 | −52.011 | 1.00 | 53.90 | A |
| ATOM | 469 | N | THR | A | 207 | −30.196 | 99.192 | −52.616 | 1.00 | 51.29 | A |
| ATOM | 470 | CA | THR | A | 207 | −29.940 | 100.037 | −53.763 | 1.00 | 48.01 | A |
| ATOM | 471 | CB | THR | A | 207 | −28.630 | 99.591 | −54.464 | 1.00 | 45.91 | A |
| ATOM | 472 | OG1 | THR | A | 207 | −27.533 | 100.326 | −53.910 | 1.00 | 44.17 | A |
| ATOM | 473 | CG2 | THR | A | 207 | −28.691 | 99.791 | −55.964 | 1.00 | 44.96 | A |
| ATOM | 474 | C | THR | A | 207 | −31.127 | 99.883 | −54.687 | 1.00 | 47.31 | A |
| ATOM | 475 | O | THR | A | 207 | −31.213 | 98.919 | −55.444 | 1.00 | 48.96 | A |
| ATOM | 476 | N | ARG | A | 208 | −32.065 | 100.816 | −54.597 | 1.00 | 45.32 | A |
| ATOM | 477 | CA | ARG | A | 208 | −33.247 | 100.764 | −55.443 | 1.00 | 44.91 | A |
| ATOM | 478 | CB | ARG | A | 208 | −34.480 | 100.402 | −54.607 | 1.00 | 44.62 | A |
| ATOM | 479 | CG | ARG | A | 208 | −34.496 | 98.948 | −54.097 | 1.00 | 46.51 | A |
| ATOM | 480 | CD | ARG | A | 208 | −35.468 | 98.036 | −54.867 | 1.00 | 47.18 | A |
| ATOM | 481 | NE | ARG | A | 208 | −35.664 | 96.753 | −54.178 | 1.00 | 48.87 | A |
| ATOM | 482 | GZ | ARG | A | 208 | −36.486 | 95.773 | −54.579 | 1.00 | 49.41 | A |
| ATOM | 483 | NH1 | ARG | A | 208 | −37.226 | 95.900 | −55.682 | 1.00 | 47.51 | A |
| ATOM | 484 | NH2 | ARG | A | 208 | −36.543 | 94.636 | −53.888 | 1.00 | 47.45 | A |
| ATOM | 485 | C | ARG | A | 208 | −33.469 | 102.075 | −56.204 | 1.00 | 43.71 | A |
| ATOM | 486 | O | ARG | A | 208 | −33.923 | 103.079 | −55.653 | 1.00 | 45.29 | A |
| ATOM | 487 | N | HIS | A | 209 | −33.140 | 102.050 | −57.488 | 1.00 | 41.05 | A |
| ATOM | 488 | CA | HIS | A | 209 | −33.291 | 103.213 | −58.333 | 1.00 | 37.67 | A |
| ATOM | 489 | CB | HIS | A | 209 | −31.922 | 103.786 | −58.624 | 1.00 | 36.08 | A |
| ATOM | 490 | CG | HIS | A | 209 | −31.957 | 105.160 | −59.199 | 1.00 | 36.05 | A |
| ATOM | 491 | CD2 | HIS | A | 209 | −31.571 | 106.348 | −58.682 | 1.00 | 36.46 | A |
| ATOM | 492 | ND1 | HIS | A | 209 | −32.424 | 105.423 | −60.464 | 1.00 | 35.06 | A |
| ATOM | 493 | CE1 | HIS | A | 209 | −32.321 | 106.716 | −60.706 | 1.00 | 38.25 | A |
| ATOM | 494 | NE2 | HIS | A | 209 | −31.805 | 107.299 | −59.641 | 1.00 | 37.40 | A |
| ATOM | 495 | C | HIS | A | 209 | −33.968 | 102.781 | −59.619 | 1.00 | 36.00 | A |
| ATOM | 496 | O | HIS | A | 209 | −33.756 | 101.663 | −60.073 | 1.00 | 36.98 | A |
| ATOM | 497 | N | PRO | A | 210 | −34.807 | 103.653 | −60.213 | 1.00 | 34.34 | A |
| ATOM | 498 | CD | PRO | A | 210 | −35.258 | 104.936 | −59.647 | 1.00 | 34.21 | A |
| ATOM | 499 | CA | PRO | A | 210 | −35.535 | 103.384 | −61.456 | 1.00 | 32.85 | A |
| ATOM | 500 | CB | PRO | A | 210 | −36.115 | 104.744 | −61.802 | 1.00 | 31.55 | A |
| ATOM | 501 | CG | PRO | A | 210 | −36.480 | 105.244 | −60.480 | 1.00 | 31.88 | A |
| ATOM | 502 | C | PRO | A | 210 | −34.743 | 102.785 | −62.606 | 1.00 | 31.89 | A |
| ATOM | 503 | O | PRO | A | 210 | −35.293 | 102.044 | −63.411 | 1.00 | 31.56 | A |
| ATOM | 504 | N | PHE | A | 211 | −33.457 | 103.100 | −62.679 | 1.00 | 30.98 | A |
| ATOM | 505 | CA | PHE | A | 211 | −32.616 | 102.590 | −63.750 | 1.00 | 30.28 | A |
| ATOM | 506 | CB | PHE | A | 211 | −31.855 | 103.747 | −64.390 | 1.00 | 29.32 | A |
| ATOM | 507 | CG | PHE | A | 211 | −32.743 | 104.900 | −64.784 | 1.00 | 27.81 | A |
| ATOM | 508 | CD1 | PHE | A | 211 | −33.840 | 104.697 | −65.616 | 1.00 | 26.67 | A |
| ATOM | 509 | CD2 | PHE | A | 211 | −32.511 | 106.179 | −64.288 | 1.00 | 26.15 | A |
| ATOM | 510 | CE1 | PHE | A | 211 | −34.675 | 105.743 | −65.930 | 1.00 | 25.33 | A |
| ATOM | 511 | CE2 | PHE | A | 211 | −33.351 | 107.227 | −64.606 | 1.00 | 23.91 | A |
| ATOM | 512 | CZ | PHE | A | 211 | −34.426 | 107.014 | −65.419 | 1.00 | 23.11 | A |
| ATOM | 513 | C | PHE | A | 211 | −31.652 | 101.506 | −63.291 | 1.00 | 30.43 | A |
| ATOM | 514 | O | PHE | A | 211 | −31.164 | 100.735 | −64.099 | 1.00 | 30.84 | A |
| ATOM | 515 | N | LEU | A | 212 | −31.377 | 101.452 | −61.992 | 1.00 | 30.56 | A |
| ATOM | 516 | CA | LEU | A | 212 | −30.484 | 100.437 | −61.450 | 1.00 | 31.26 | A |
| ATOM | 517 | CB | LEU | A | 212 | −29.911 | 100.876 | −60.098 | 1.00 | 31.17 | A |
| ATOM | 518 | CG | LEU | A | 212 | −28.721 | 101.844 | −60.235 | 1.00 | 31.98 | A |
| ATOM | 519 | CD1 | LEU | A | 212 | −28.476 | 102.553 | −58.930 | 1.00 | 30.89 | A |
| ATOM | 520 | CD2 | LEU | A | 212 | −27.471 | 101.084 | −60.703 | 1.00 | 29.63 | A |
| ATOM | 521 | C | LEU | A | 212 | −31.313 | 99.191 | −61.305 | 1.00 | 30.74 | A |
| ATOM | 522 | O | LEU | A | 212 | −32.472 | 99.280 | −60.931 | 1.00 | 31.25 | A |
| ATOM | 523 | N | THR | A | 213 | −30.735 | 98.035 | −61.615 | 1.00 | 30.78 | A |
| ATOM | 524 | CA | THR | A | 213 | −31.471 | 96.785 | −61.544 | 1.00 | 31.45 | A |
| ATOM | 525 | CB | THR | A | 213 | −30.670 | 95.657 | −62.167 | 1.00 | 31.33 | A |
| ATOM | 526 | OG1 | THR | A | 213 | −30.131 | 96.097 | −63.416 | 1.00 | 30.27 | A |
| ATOM | 527 | CG2 | THR | A | 213 | −31.568 | 94.452 | −62.416 | 1.00 | 31.83 | A |
| ATOM | 528 | C | THR | A | 213 | −31.836 | 96.414 | −60.118 | 1.00 | 32.46 | A |
| ATOM | 529 | O | THR | A | 213 | −30.978 | 96.374 | −59.236 | 1.00 | 33.33 | A |
| ATOM | 530 | N | ALA | A | 214 | −33.114 | 96.136 | −59.894 | 1.00 | 33.86 | A |
| ATOM | 531 | CA | ALA | A | 214 | −33.589 | 95.805 | −58.558 | 1.00 | 35.77 | A |
| ATOM | 532 | CB | ALA | A | 214 | −35.061 | 96.188 | −58.400 | 1.00 | 35.44 | A |
| ATOM | 533 | C | ALA | A | 214 | −33.409 | 94.353 | −58.243 | 1.00 | 37.09 | A |
| ATOM | 534 | O | ALA | A | 214 | −33.470 | 93.509 | −59.119 | 1.00 | 38.14 | A |
| ATOM | 535 | N | LEU | A | 215 | −33.183 | 94.077 | −56.972 | 1.00 | 39.49 | A |
| ATOM | 536 | CA | LEU | A | 215 | −33.010 | 92.722 | −56.486 | 1.00 | 42.45 | A |
| ATOM | 537 | CB | LEU | A | 215 | −31.897 | 92.697 | −55.446 | 1.00 | 42.72 | A |
| ATOM | 538 | CG | LEU | A | 215 | −31.888 | 91.514 | −54.492 | 1.00 | 43.40 | A |
| ATOM | 539 | CD1 | LEU | A | 215 | −32.051 | 90.237 | −55.274 | 1.00 | 45.35 | A |
| ATOM | 540 | CD2 | LEU | A | 215 | −30.590 | 91.511 | −53.709 | 1.00 | 43.94 | A |
| ATOM | 541 | C | LEU | A | 215 | −34.321 | 92.304 | −55.850 | 1.00 | 44.19 | A |
| ATOM | 542 | O | LEU | A | 215 | −34.812 | 92.989 | −54.952 | 1.00 | 44.78 | A |
| ATOM | 543 | N | LYS | A | 216 | −34.900 | 91.201 | −56.315 | 1.00 | 46.18 | A |
| ATOM | 544 | CA | LYS | A | 216 | −36.166 | 90.735 | −55.751 | 1.00 | 48.67 | A |
| ATOM | 545 | CB | LYS | A | 216 | −36.859 | 89.819 | −56.746 | 1.00 | 48.50 | A |
| ATOM | 546 | CG | LYS | A | 216 | −37.205 | 90.520 | −58.031 | 1.00 | 51.05 | A |
| ATOM | 547 | CD | LYS | A | 216 | −38.413 | 91.434 | −57.869 | 1.00 | 51.75 | A |
| ATOM | 548 | CE | LYS | A | 216 | −38.779 | 92.107 | −59.191 | 1.00 | 50.82 | A |
| ATOM | 549 | NZ | LYS | A | 216 | −40.056 | 92.858 | −59.069 | 1.00 | 49.52 | A |
| ATOM | 550 | C | LYS | A | 216 | −35.950 | 90.021 | −54.408 | 1.00 | 49.81 | A |
| ATOM | 551 | O | LYS | A | 216 | −36.675 | 90.257 | −53.431 | 1.00 | 49.27 | A |
| ATOM | 552 | N | TYR | A | 217 | −34.941 | 89.157 | −54.369 | 1.00 | 50.73 | A |
| ATOM | 553 | CA | TYR | A | 217 | −34.588 | 88.430 | −53.163 | 1.00 | 52.36 | A |
| ATOM | 554 | CB | TYR | A | 217 | −35.766 | 87.590 | −52.652 | 1.00 | 54.56 | A |
| ATOM | 555 | CG | TYR | A | 217 | −36.528 | 86.787 | −53.683 | 1.00 | 55.56 | A |
| ATOM | 556 | CD1 | TYR | A | 217 | −37.831 | 86.359 | −53.416 | 1.00 | 57.36 | A |
| ATOM | 557 | CE1 | TYR | A | 217 | −38.559 | 85.610 | −54.340 | 1.00 | 57.83 | A |
| ATOM | 558 | CD2 | TYR | A | 217 | −35.964 | 86.445 | −54.907 | 1.00 | 55.19 | A |
| ATOM | 559 | CE2 | TYR | A | 217 | −36.684 | 85.694 | −55.844 | 1.00 | 56.62 | A |
| ATOM | 560 | CZ | TYR | A | 217 | −37.982 | 85.279 | −55.551 | 1.00 | 57.77 | A |
| ATOM | 561 | OH | TYR | A | 217 | −38.708 | 84.525 | −56.452 | 1.00 | 57.86 | A |
| ATOM | 562 | C | TYR | A | 217 | −33.369 | 87.554 | −53.353 | 1.00 | 52.04 | A |
| ATOM | 563 | O | TYR | A | 217 | −33.127 | 87.033 | −54.432 | 1.00 | 52.56 | A |
| ATOM | 564 | N | ALA | A | 218 | −32.592 | 87.414 | −52.290 | 1.00 | 51.08 | A |
| ATOM | 565 | CA | ALA | A | 218 | −31.393 | 86.600 | −52.331 | 1.00 | 50.68 | A |
| ATOM | 566 | CB | ALA | A | 218 | −30.199 | 87.469 | −52.087 | 1.00 | 51.45 | A |
| ATOM | 567 | C | ALA | A | 218 | −31.465 | 85.512 | −51.269 | 1.00 | 50.45 | A |
| ATOM | 568 | O | ALA | A | 218 | −31.645 | 85.807 | −50.096 | 1.00 | 50.53 | A |
| ATOM | 569 | N | PHE | A | 219 | −31.337 | 84.255 | −51.671 | 1.00 | 50.36 | A |
| ATOM | 570 | CA | PHE | A | 219 | −31.373 | 83.184 | −50.695 | 1.00 | 51.82 | A |
| ATOM | 571 | CB | PHE | A | 219 | −32.354 | 82.086 | −51.119 | 1.00 | 53.27 | A |
| ATOM | 572 | CG | PHE | A | 219 | −32.181 | 81.606 | −52.527 | 1.00 | 54.08 | A |
| ATOM | 573 | CD1 | PHE | A | 219 | −32.609 | 82.379 | −53.595 | 1.00 | 55.73 | A |
| ATOM | 574 | CD2 | PHE | A | 219 | −31.638 | 80.353 | −52.781 | 1.00 | 54.53 | A |
| ATOM | 575 | CE1 | PHE | A | 219 | −32.507 | 81.907 | −54.903 | 1.00 | 57.06 | A |
| ATOM | 576 | CE2 | PHE | A | 219 | −31.529 | 79.872 | −54.072 | 1.00 | 55.44 | A |
| ATOM | 577 | CZ | PHE | A | 219 | −31.966 | 80.648 | −55.141 | 1.00 | 56.54 | A |
| ATOM | 578 | C | PHE | A | 219 | −29.994 | 82.586 | −50.389 | 1.00 | 52.37 | A |
| ATOM | 579 | O | PHE | A | 219 | −28.991 | 83.302 | −50.375 | 1.00 | 52.39 | A |
| ATOM | 580 | N | GLN | A | 220 | −29.942 | 81.284 | −50.129 | 1.00 | 53.26 | A |
| ATOM | 581 | CA | GLN | A | 220 | −28.673 | 80.639 | −49.801 | 1.00 | 54.94 | A |
| ATOM | 582 | CB | GLN | A | 220 | −28.453 | 80.665 | −48.282 | 1.00 | 56.53 | A |
| ATOM | 583 | CG | GLN | A | 220 | −28.704 | 82.003 | −47.585 | 1.00 | 59.25 | A |
| ATOM | 584 | CD | GLN | A | 220 | −27.470 | 82.902 | −47.524 | 1.00 | 60.67 | A |
| ATOM | 585 | OE1 | GLN | A | 220 | −26.378 | 82.461 | −47.101 | 1.00 | 60.21 | A |
| ATOM | 586 | NE2 | GLN | A | 220 | −27.637 | 84.179 | −47.931 | 1.00 | 57.63 | A |
| ATOM | 587 | C | GLN | A | 220 | −28.559 | 79.183 | −50.271 | 1.00 | 54.81 | A |
| ATOM | 588 | O | GLN | A | 220 | −29.509 | 78.590 | −50.763 | 1.00 | 54.81 | A |
| ATOM | 589 | N | THR | A | 221 | −27.364 | 78.632 | −50.090 | 1.00 | 54.74 | A |
| ATOM | 590 | CA | THR | A | 221 | −27.000 | 77.252 | −50.417 | 1.00 | 54.03 | A |
| ATOM | 591 | CB | THR | A | 221 | −26.771 | 77.043 | −51.972 | 1.00 | 54.42 | A |
| ATOM | 592 | OG1 | THR | A | 221 | −27.944 | 76.463 | −52.550 | 1.00 | 53.92 | A |
| ATOM | 593 | CG2 | THR | A | 221 | −25.577 | 76.110 | −52.279 | 1.00 | 52.97 | A |
| ATOM | 594 | C | THR | A | 221 | −25.686 | 77.081 | −49.661 | 1.00 | 53.41 | A |
| ATOM | 595 | O | THR | A | 221 | −24.745 | 77.813 | −49.943 | 1.00 | 54.69 | A |
| ATOM | 596 | N | HIS | A | 222 | −25.632 | 76.169 | −48.684 | 1.00 | 51.78 | A |
| ATOM | 597 | CA | HIS | A | 222 | −24.404 | 75.911 | −47.898 | 1.00 | 50.07 | A |
| ATOM | 598 | CB | HIS | A | 222 | −24.414 | 74.515 | −47.252 | 1.00 | 53.13 | A |
| ATOM | 599 | CG | HIS | A | 222 | −24.788 | 74.504 | −45.804 | 1.00 | 55.35 | A |
| ATOM | 600 | CD2 | HIS | A | 222 | −25.233 | 73.501 | −45.009 | 1.00 | 56.31 | A |
| ATOM | 601 | ND1 | HIS | A | 222 | −24.654 | 75.608 | −44.990 | 1.00 | 55.97 | A |
| ATOM | 602 | CE1 | HIS | A | 222 | −24.999 | 75.286 | −43.755 | 1.00 | 56.19 | A |
| ATOM | 603 | NE2 | HIS | A | 222 | −25.353 | 74.013 | −43.740 | 1.00 | 57.22 | A |
| ATOM | 604 | C | HIS | A | 222 | −23.171 | 75.973 | −48.780 | 1.00 | 46.06 | A |
| ATOM | 605 | O | HIS | A | 222 | −22.049 | 75.997 | −48.296 | 1.00 | 44.34 | A |
| ATOM | 606 | N | ASP | A | 223 | −23.401 | 76.003 | −50.081 | 1.00 | 42.83 | A |
| ATOM | 607 | CA | ASP | A | 223 | −22.333 | 76.036 | −51.046 | 1.00 | 40.84 | A |
| ATOM | 608 | CB | ASP | A | 223 | −22.418 | 74.743 | −51.854 | 1.00 | 42.11 | A |
| ATOM | 609 | CG | ASP | A | 223 | −21.231 | 74.530 | −52.736 | 1.00 | 41.53 | A |
| ATOM | 610 | OD1 | ASP | A | 223 | −21.373 | 74.719 | −53.971 | 1.00 | 41.16 | A |
| ATOM | 611 | OD2 | ASP | A | 223 | −20.165 | 74.178 | −52.179 | 1.00 | 40.46 | A |
| ATOM | 612 | C | ASP | A | 223 | −22.328 | 77.262 | −51.979 | 1.00 | 39.06 | A |
| ATOM | 613 | O | ASP | A | 223 | −21.336 | 77.511 | −52.652 | 1.00 | 38.32 | A |
| ATOM | 614 | N | ARG | A | 224 | −23.414 | 78.033 | −52.024 | 1.00 | 37.35 | A |
| ATOM | 615 | CA | ARG | A | 224 | −23.466 | 79.193 | −52.916 | 1.00 | 35.45 | A |
| ATOM | 616 | CB | ARG | A | 224 | −23.831 | 78.755 | −54.333 | 1.00 | 34.31 | A |
| ATOM | 617 | CG | ARG | A | 224 | −22.897 | 77.708 | −54.855 | 1.00 | 32.43 | A |
| ATOM | 618 | CD | ARG | A | 224 | −23.154 | 77.302 | −56.261 | 1.00 | 30.11 | A |
| ATOM | 619 | NE | ARG | A | 224 | −22.237 | 76.212 | −56.568 | 1.00 | 29.45 | A |
| ATOM | 620 | CZ | ARG | A | 224 | −21.945 | 75.788 | −57.787 | 1.00 | 29.32 | A |
| ATOM | 621 | NH1 | ARG | A | 224 | −22.489 | 76.350 | −58.863 | 1.00 | 28.21 | A |
| ATOM | 622 | NH2 | ARG | A | 224 | −21.090 | 74.798 | −57.925 | 1.00 | 29.74 | A |
| ATOM | 623 | C | ARG | A | 224 | −24.415 | 80.283 | −52.496 | 1.00 | 34.18 | A |
| ATOM | 624 | O | ARG | A | 224 | −25.244 | 80.082 | −51.630 | 1.00 | 34.36 | A |
| ATOM | 625 | N | LEU | A | 225 | −24.274 | 81.447 | −53.121 | 1.00 | 34.49 | A |
| ATOM | 626 | CA | LEU | A | 225 | −25.127 | 82.603 | −52.834 | 1.00 | 34.86 | A |
| ATOM | 627 | CB | LEU | A | 225 | −24.279 | 83.820 | −52.415 | 1.00 | 34.45 | A |
| ATOM | 628 | CG | LEU | A | 225 | −23.428 | 83.696 | −51.143 | 1.00 | 34.20 | A |
| ATOM | 629 | CD1 | LEU | A | 225 | −22.561 | 84.903 | −50.996 | 1.00 | 32.85 | A |
| ATOM | 630 | CD2 | LEU | A | 225 | −24.315 | 83.543 | −49.922 | 1.00 | 33.81 | A |
| ATOM | 631 | C | LEU | A | 225 | −25.924 | 82.928 | −54.095 | 1.00 | 34.83 | A |
| ATOM | 632 | O | LEU | A | 225 | −25.350 | 83.164 | −55.155 | 1.00 | 34.55 | A |
| ATOM | 633 | N | CYS | A | 226 | −27.245 | 82.935 | −53.974 | 1.00 | 34.97 | A |
| ATOM | 634 | CA | CYS | A | 226 | −28.097 | 83.198 | −55.116 | 1.00 | 35.79 | A |
| ATOM | 635 | CB | CYS | A | 226 | −29.102 | 82.064 | −55.285 | 1.00 | 36.99 | A |
| ATOM | 636 | SG | CYS | A | 226 | −28.374 | 80.414 | −55.213 | 1.00 | 40.23 | A |
| ATOM | 637 | C | CYS | A | 226 | −28.846 | 84.514 | −55.030 | 1.00 | 35.90 | A |
| ATOM | 638 | O | CYS | A | 226 | −29.587 | 84.768 | −54.083 | 1.00 | 36.05 | A |
| ATOM | 639 | N | PHE | A | 227 | −28.653 | 85.335 | −56.055 | 1.00 | 35.75 | A |
| ATOM | 640 | CA | PHE | A | 227 | −29.294 | 86.635 | −56.165 | 1.00 | 33.68 | A |
| ATOM | 641 | CB | PHE | A | 227 | −28.240 | 87.746 | −56.292 | 1.00 | 32.87 | A |
| ATOM | 642 | CG | PHE | A | 227 | −27.259 | 87.775 | −55.161 | 1.00 | 30.58 | A |
| ATOM | 643 | CD1 | PHE | A | 227 | −25.943 | 87.419 | −55.359 | 1.00 | 30.80 | A |
| ATOM | 644 | CD2 | PHE | A | 227 | −27.672 | 88.101 | −53.880 | 1.00 | 28.54 | A |
| ATOM | 645 | CE1 | PHE | A | 227 | −25.064 | 87.387 | −54.299 | 1.00 | 30.44 | A |
| ATOM | 646 | CE2 | PHE | A | 227 | −26.801 | 88.069 | −52.824 | 1.00 | 26.58 | A |
| ATOM | 647 | CZ | PHE | A | 227 | −25.498 | 87.710 | −53.031 | 1.00 | 29.42 | A |
| ATOM | 648 | C | PHE | A | 227 | −30.179 | 86.640 | −57.393 | 1.00 | 33.39 | A |
| ATOM | 649 | O | PHE | A | 227 | −29.693 | 86.616 | −58.522 | 1.00 | 33.00 | A |
| ATOM | 650 | N | VAL | A | 228 | −31.483 | 86.649 | −57.170 | 1.00 | 32.74 | A |
| ATOM | 651 | CA | VAL | A | 228 | −32.427 | 86.688 | −58.273 | 1.00 | 32.72 | A |
| ATOM | 652 | CB | VAL | A | 228 | −33.711 | 85.891 | −57.933 | 1.00 | 30.83 | A |
| ATOM | 653 | CG1 | VAL | A | 228 | −34.583 | 85.757 | −59.152 | 1.00 | 28.11 | A |
| ATOM | 654 | CG2 | VAL | A | 228 | −33.336 | 84.513 | −57.403 | 1.00 | 28.93 | A |
| ATOM | 655 | C | VAL | A | 228 | −32.735 | 88.168 | −58.478 | 1.00 | 34.22 | A |
| ATOM | 656 | O | VAL | A | 228 | −33.300 | 88.816 | −57.602 | 1.00 | 34.89 | A |
| ATOM | 657 | N | MET | A | 229 | −32.333 | 88.700 | −59.630 | 1.00 | 35.98 | A |
| ATOM | 658 | CA | MET | A | 229 | −32.533 | 90.115 | −59.956 | 1.00 | 37.87 | A |
| ATOM | 659 | CB | MET | A | 229 | −31.196 | 90.736 | −60.380 | 1.00 | 38.28 | A |
| ATOM | 660 | CG | MET | A | 229 | −30.046 | 90.401 | −59.456 | 1.00 | 41.11 | A |
| ATOM | 661 | SD | MET | A | 229 | −28.403 | 90.651 | −60.191 | 1.00 | 47.29 | A |
| ATOM | 662 | CE | MET | A | 229 | −28.058 | 89.073 | −60.903 | 1.00 | 43.74 | A |
| ATOM | 663 | C | MET | A | 229 | −33.561 | 90.338 | −61.072 | 1.00 | 37.89 | A |
| ATOM | 664 | O | MET | A | 229 | −33.741 | 89.495 | −61.942 | 1.00 | 37.51 | A |
| ATOM | 665 | N | GLU | A | 230 | −34.235 | 91.482 | −61.047 | 1.00 | 39.52 | A |
| ATOM | 666 | CA | GLU | A | 230 | −35.203 | 91.792 | −62.087 | 1.00 | 41.39 | A |
| ATOM | 667 | CB | GLU | A | 230 | −35.859 | 93.151 | −61.847 | 1.00 | 42.98 | A |
| ATOM | 668 | CG | GLU | A | 230 | −34.856 | 94.304 | −61.836 | 1.00 | 48.22 | A |
| ATOM | 669 | CD | GLU | A | 230 | −35.389 | 95.606 | −62.449 | 1.00 | 52.74 | A |
| ATOM | 670 | OE1 | GLU | A | 230 | −35.741 | 95.615 | −63.659 | 1.00 | 52.91 | A |
| ATOM | 671 | OE2 | GLU | A | 230 | −35.441 | 96.624 | −61.717 | 1.00 | 53.57 | A |
| ATOM | 672 | C | GLU | A | 230 | −34.388 | 91.852 | −63.366 | 1.00 | 42.15 | A |
| ATOM | 673 | O | GLU | A | 230 | −33.413 | 92.604 | −63.456 | 1.00 | 41.51 | A |
| ATOM | 674 | N | TYR | A | 231 | −34.776 | 91.048 | −64.349 | 1.00 | 43.05 | A |
| ATOM | 675 | CA | TYR | A | 231 | −34.081 | 91.021 | −65.632 | 1.00 | 43.46 | A |
| ATOM | 676 | CB | TYR | A | 231 | −33.912 | 89.554 | −66.066 | 1.00 | 44.83 | A |
| ATOM | 677 | CG | TYR | A | 231 | −34.060 | 89.274 | −67.538 | 1.00 | 47.32 | A |
| ATOM | 678 | CD1 | TYR | A | 231 | −33.036 | 89.556 | −68.442 | 1.00 | 47.92 | A |
| ATOM | 679 | CE1 | TYR | A | 231 | −33.201 | 89.330 | −69.810 | 1.00 | 48.98 | A |
| ATOM | 680 | CD2 | TYR | A | 231 | −35.247 | 88.754 | −68.033 | 1.00 | 49.05 | A |
| ATOM | 681 | CE2 | TYR | A | 231 | −35.425 | 88.525 | −69.389 | 1.00 | 51.23 | A |
| ATOM | 682 | CZ | TYR | A | 231 | −34.409 | 88.816 | −70.277 | 1.00 | 50.72 | A |
| ATOM | 683 | OH | TYR | A | 231 | −34.647 | 88.622 | −71.624 | 1.00 | 52.16 | A |
| ATOM | 684 | C | TYR | A | 231 | −34.802 | 91.854 | −66.705 | 1.00 | 42.58 | A |
| ATOM | 685 | O | TYR | A | 231 | −35.984 | 91.646 | −66.976 | 1.00 | 42.89 | A |
| ATOM | 686 | N | ALA | A | 232 | −34.088 | 92.814 | −67.290 | 1.00 | 42.00 | A |
| ATOM | 687 | CA | ALA | A | 232 | −34.637 | 93.653 | −68.356 | 1.00 | 41.81 | A |
| ATOM | 688 | CB | ALA | A | 232 | −33.527 | 94.416 | −69.054 | 1.00 | 41.02 | A |
| ATOM | 689 | C | ALA | A | 232 | −35.344 | 92.749 | −69.351 | 1.00 | 42.16 | A |
| ATOM | 690 | O | ALA | A | 232 | −35.270 | 91.537 | −69.244 | 1.00 | 43.74 | A |
| ATOM | 691 | N | ASN | A | 233 | −36.008 | 93.323 | −70.343 | 1.00 | 43.23 | A |
| ATOM | 692 | CA | ASN | A | 233 | −36.753 | 92.492 | −71.288 | 1.00 | 43.75 | A |
| ATOM | 693 | CB | ASN | A | 233 | −38.202 | 92.344 | −70.796 | 1.00 | 45.51 | A |
| ATOM | 694 | CG | ASN | A | 233 | −38.853 | 91.048 | −71.237 | 1.00 | 46.48 | A |
| ATOM | 695 | OD1 | ASN | A | 233 | −38.324 | 89.961 | −71.018 | 1.00 | 48.18 | A |
| ATOM | 696 | ND2 | ASN | A | 233 | −40.025 | 91.160 | −71.840 | 1.00 | 47.41 | A |
| ATOM | 697 | C | ASN | A | 233 | −36.751 | 93.075 | −72.683 | 1.00 | 42.66 | A |
| ATOM | 698 | O | ASN | A | 233 | −37.114 | 92.398 | −73.642 | 1.00 | 43.49 | A |
| ATOM | 699 | N | GLY | A | 234 | −36.343 | 94.331 | −72.795 | 1.00 | 41.69 | A |
| ATOM | 700 | CA | GLY | A | 234 | −36.326 | 94.961 | −74.094 | 1.00 | 41.95 | A |
| ATOM | 701 | C | GLY | A | 234 | −34.963 | 95.268 | −74.676 | 1.00 | 41.82 | A |
| ATOM | 702 | O | GLY | A | 234 | −34.661 | 96.423 | −74.955 | 1.00 | 41.22 | A |
| ATOM | 703 | N | GLY | A | 235 | −34.125 | 94.250 | −74.834 | 1.00 | 42.45 | A |
| ATOM | 704 | CA | GLY | A | 235 | −32.826 | 94.469 | −75.442 | 1.00 | 44.17 | A |
| ATOM | 705 | C | GLY | A | 235 | −31.569 | 94.772 | −74.641 | 1.00 | 45.65 | A |
| ATOM | 706 | O | GLY | A | 235 | −31.596 | 94.997 | −73.433 | 1.00 | 45.42 | A |
| ATOM | 707 | N | GLU | A | 236 | −30.452 | 94.788 | −75.370 | 1.00 | 46.13 | A |
| ATOM | 708 | CA | GLU | A | 236 | −29.123 | 95.041 | −74.831 | 1.00 | 45.84 | A |
| ATOM | 709 | CB | GLU | A | 236 | −28.246 | 93.821 | −75.073 | 1.00 | 46.16 | A |
| ATOM | 710 | CG | GLU | A | 236 | −29.012 | 92.502 | −75.061 | 1.00 | 49.28 | A |
| ATOM | 711 | CD | GLU | A | 236 | −29.170 | 91.927 | −73.661 | 1.00 | 52.42 | A |
| ATOM | 712 | OE1 | GLU | A | 236 | −28.220 | 91.263 | −73.170 | 1.00 | 53.46 | A |
| ATOM | 713 | OE2 | GLU | A | 236 | −30.240 | 92.150 | −73.050 | 1.00 | 52.08 | A |
| ATOM | 714 | C | GLU | A | 236 | −28.543 | 96.224 | −75.582 | 1.00 | 45.41 | A |
| ATOM | 715 | O | GLU | A | 236 | −28.093 | 96.081 | −76.707 | 1.00 | 45.48 | A |
| ATOM | 716 | N | LEU | A | 237 | −28.557 | 97.395 | −74.966 | 1.00 | 46.49 | A |
| ATOM | 717 | CA | LEU | A | 237 | −28.030 | 98.593 | −75.600 | 1.00 | 46.92 | A |
| ATOM | 718 | CB | LEU | A | 237 | −27.730 | 99.635 | −74.539 | 1.00 | 46.39 | A |
| ATOM | 719 | CG | LEU | A | 237 | −27.505 | 101.045 | −75.072 | 1.00 | 49.33 | A |
| ATOM | 720 | CD1 | LEU | A | 237 | −27.954 | 102.038 | −74.010 | 1.00 | 50.02 | A |
| ATOM | 721 | CD2 | LEU | A | 237 | −26.036 | 101.251 | −75.459 | 1.00 | 50.55 | A |
| ATOM | 722 | C | LEU | A | 237 | −26.781 | 98.312 | −76.435 | 1.00 | 48.20 | A |
| ATOM | 723 | O | LEU | A | 237 | −26.615 | 98.854 | −77.519 | 1.00 | 49.31 | A |
| ATOM | 724 | N | PHE | A | 238 | −25.901 | 97.455 | −75.944 | 1.00 | 50.29 | A |
| ATOM | 725 | CA | PHE | A | 238 | −24.702 | 97.138 | −76.699 | 1.00 | 51.06 | A |
| ATOM | 726 | CB | PHE | A | 238 | −23.923 | 96.005 | −76.042 | 1.00 | 52.21 | A |
| ATOM | 727 | CG | PHE | A | 238 | −22.663 | 95.653 | −76.776 | 1.00 | 55.02 | A |
| ATOM | 728 | CD1 | PHE | A | 238 | −21.471 | 96.325 | −76.507 | 1.00 | 56.03 | A |
| ATOM | 729 | CD2 | PHE | A | 238 | −22.678 | 94.694 | −77.785 | 1.00 | 54.88 | A |
| ATOM | 730 | CE1 | PHE | A | 238 | −20.317 | 96.050 | −77.231 | 1.00 | 56.01 | A |
| ATOM | 731 | CE2 | PHE | A | 238 | −21.528 | 94.412 | −78.518 | 1.00 | 55.49 | A |
| ATOM | 732 | CZ | PHE | A | 238 | −20.347 | 95.092 | −78.240 | 1.00 | 56.77 | A |
| ATOM | 733 | C | PHE | A | 238 | −25.088 | 96.714 | −78.111 | 1.00 | 51.64 | A |
| ATOM | 734 | O | PHE | A | 238 | −24.487 | 97.167 | −79.086 | 1.00 | 51.78 | A |
| ATOM | 735 | N | PHE | A | 239 | −26.086 | 95.838 | −78.214 | 1.00 | 51.05 | A |
| ATOM | 736 | CA | PHE | A | 239 | −26.541 | 95.355 | −79.508 | 1.00 | 50.34 | A |
| ATOM | 737 | CB | PHE | A | 239 | −27.068 | 93.930 | −79.400 | 1.00 | 53.30 | A |
| ATOM | 738 | CG | PHE | A | 239 | −25.976 | 92.886 | −79.378 | 1.00 | 55.81 | A |
| ATOM | 739 | CD1 | PHE | A | 239 | −26.073 | 91.773 | −78.543 | 1.00 | 55.46 | A |
| ATOM | 740 | CD2 | PHE | A | 239 | −24.833 | 93.040 | −80.171 | 1.00 | 55.37 | A |
| ATOM | 741 | CE1 | PHE | A | 239 | −25.057 | 90.842 | −78.493 | 1.00 | 56.34 | A |
| ATOM | 742 | CE2 | PHE | A | 239 | −23.817 | 92.118 | −80.130 | 1.00 | 56.41 | A |
| ATOM | 743 | CZ | PHE | A | 239 | −23.919 | 91.008 | −79.286 | 1.00 | 57.26 | A |
| ATOM | 744 | C | PHE | A | 239 | −27.579 | 96.245 | −80.138 | 1.00 | 49.66 | A |
| ATOM | 745 | O | PHE | A | 239 | −27.938 | 96.051 | −81.297 | 1.00 | 50.84 | A |
| ATOM | 746 | N | HIS | A | 240 | −28.074 | 97.216 | −79.375 | 1.00 | 47.54 | A |
| ATOM | 747 | CA | HIS | A | 240 | −29.033 | 98.174 | −79.905 | 1.00 | 43.90 | A |
| ATOM | 748 | CB | HIS | A | 240 | −29.819 | 98.842 | −78.786 | 1.00 | 42.16 | A |
| ATOM | 749 | CG | HIS | A | 240 | −30.954 | 98.016 | −78.283 | 1.00 | 41.30 | A |
| ATOM | 750 | CD2 | HIS | A | 240 | −31.186 | 97.465 | −77.070 | 1.00 | 41.33 | A |
| ATOM | 751 | ND1 | HIS | A | 240 | −32.008 | 97.641 | −79.085 | 1.00 | 41.24 | A |
| ATOM | 752 | CE1 | HIS | A | 240 | −32.840 | 96.889 | −78.389 | 1.00 | 41.90 | A |
| ATOM | 753 | NE2 | HIS | A | 240 | −32.365 | 96.767 | −77.163 | 1.00 | 40.71 | A |
| ATOM | 754 | C | HIS | A | 240 | −28.202 | 99.209 | −80.643 | 1.00 | 43.45 | A |
| ATOM | 755 | O | HIS | A | 240 | −28.709 | 99.953 | −81.469 | 1.00 | 45.30 | A |
| ATOM | 756 | N | LEU | A | 241 | −26.914 | 99.244 | −80.334 | 1.00 | 41.54 | A |
| ATOM | 757 | CA | LEU | A | 241 | −25.995 | 100.161 | −80.985 | 1.00 | 41.67 | A |
| ATOM | 758 | CB | LEU | A | 241 | −24.853 | 100.506 | −80.041 | 1.00 | 39.70 | A |
| ATOM | 759 | CG | LEU | A | 241 | −23.861 | 101.533 | −80.562 | 1.00 | 39.41 | A |
| ATOM | 760 | CD1 | LEU | A | 241 | −24.539 | 102.894 | −80.572 | 1.00 | 38.38 | A |
| ATOM | 761 | CD2 | LEU | A | 241 | −22.608 | 101.545 | −79.684 | 1.00 | 37.59 | A |
| ATOM | 762 | C | LEU | A | 241 | −25.427 | 99.488 | −82.240 | 1.00 | 43.22 | A |
| ATOM | 763 | O | LEU | A | 241 | −25.372 | 100.083 | −83.309 | 1.00 | 44.67 | A |
| ATOM | 764 | N | SER | A | 242 | −25.005 | 98.239 | −82.094 | 1.00 | 42.62 | A |
| ATOM | 765 | CA | SER | A | 242 | −24.444 | 97.459 | −83.183 | 1.00 | 41.93 | A |
| ATOM | 766 | CB | SER | A | 242 | −24.279 | 96.028 | −82.716 | 1.00 | 42.54 | A |
| ATOM | 767 | OG | SER | A | 242 | −25.475 | 95.622 | −82.079 | 1.00 | 44.02 | A |
| ATOM | 768 | C | SER | A | 242 | −25.302 | 97.475 | −84.442 | 1.00 | 42.68 | A |
| ATOM | 769 | O | SER | A | 242 | −24.777 | 97.353 | −85.552 | 1.00 | 44.22 | A |
| ATOM | 770 | N | ARG | A | 243 | −26.619 | 97.590 | −84.279 | 1.00 | 42.79 | A |
| ATOM | 771 | CA | ARG | A | 243 | −27.517 | 97.634 | −85.434 | 1.00 | 41.20 | A |
| ATOM | 772 | CB | ARG | A | 243 | −28.949 | 97.291 | −85.045 | 1.00 | 40.35 | A |
| ATOM | 773 | CG | ARG | A | 243 | −29.162 | 95.943 | −84.408 | 1.00 | 41.66 | A |
| ATOM | 774 | CD | ARG | A | 243 | −30.664 | 95.623 | −84.346 | 1.00 | 42.58 | A |
| ATOM | 775 | NE | ARG | A | 243 | −31.505 | 96.805 | −84.111 | 1.00 | 43.70 | A |
| ATOM | 776 | CZ | ARG | A | 243 | −31.561 | 97.501 | −82.974 | 1.00 | 44.52 | A |
| ATOM | 777 | NH1 | ARG | A | 243 | −30.825 | 97.147 | −81.928 | 1.00 | 44.45 | A |
| ATOM | 778 | NH2 | ARG | A | 243 | −32.345 | 98.572 | −82.890 | 1.00 | 44.14 | A |
| ATOM | 779 | C | ARG | A | 243 | −27.540 | 99.030 | −86.037 | 1.00 | 40.60 | A |
| ATOM | 780 | O | ARG | A | 243 | −27.837 | 99.205 | −87.213 | 1.00 | 40.76 | A |
| ATOM | 781 | N | GLU | A | 244 | −27.218 | 100.027 | −85.227 | 1.00 | 39.12 | A |
| ATOM | 782 | CA | GLU | A | 244 | −27.255 | 101.401 | −85.682 | 1.00 | 38.02 | A |
| ATOM | 783 | CB | GLU | A | 244 | −28.285 | 102.148 | −84.861 | 1.00 | 37.26 | A |
| ATOM | 784 | CG | GLU | A | 244 | −29.634 | 101.496 | −84.936 | 1.00 | 34.08 | A |
| ATOM | 785 | CD | GLU | A | 244 | −30.661 | 102.241 | −84.156 | 1.00 | 32.02 | A |
| ATOM | 786 | OE1 | GLU | A | 244 | −30.710 | 102.072 | −82.922 | 1.00 | 30.18 | A |
| ATOM | 787 | OE2 | GLU | A | 244 | −31.414 | 103.004 | −84.787 | 1.00 | 31.62 | A |
| ATOM | 788 | C | GLU | A | 244 | −25.938 | 102.114 | −85.581 | 1.00 | 38.67 | A |
| ATOM | 789 | O | GLU | A | 244 | −25.848 | 103.296 | −85.872 | 1.00 | 38.98 | A |
| ATOM | 790 | N | ARG | A | 245 | −24.914 | 101.369 | −85.195 | 1.00 | 39.02 | A |
| ATOM | 791 | CA | ARG | A | 245 | −23.561 | 101.884 | −84.990 | 1.00 | 38.77 | A |
| ATOM | 792 | CB | ARG | A | 245 | −22.731 | 101.873 | −86.282 | 1.00 | 38.30 | A |
| ATOM | 793 | CG | ARG | A | 245 | −23.443 | 102.207 | −87.559 | 1.00 | 39.67 | A |
| ATOM | 794 | CD | ARG | A | 245 | −24.332 | 101.049 | −88.001 | 1.00 | 41.10 | A |
| ATOM | 795 | NE | ARG | A | 245 | −24.271 | 100.822 | −89.443 | 1.00 | 41.51 | A |
| ATOM | 796 | CZ | ARG | A | 245 | −23.488 | 99.920 | −90.022 | 1.00 | 42.11 | A |
| ATOM | 797 | NH1 | ARG | A | 245 | −22.697 | 99.151 | −89.288 | 1.00 | 43.11 | A |
| ATOM | 798 | NH2 | ARG | A | 245 | −23.503 | 99.777 | −91.339 | 1.00 | 42.47 | A |
| ATOM | 799 | C | ARG | A | 245 | −23.419 | 103.246 | −84.334 | 1.00 | 37.39 | A |
| ATOM | 800 | O | ARG | A | 245 | −22.472 | 103.450 | −83.597 | 1.00 | 39.64 | A |
| ATOM | 801 | N | VAL | A | 246 | −24.323 | 104.179 | −84.579 | 1.00 | 34.06 | A |
| ATOM | 802 | CA | VAL | A | 246 | −24.168 | 105.475 | −83.961 | 1.00 | 33.86 | A |
| ATOM | 803 | CB | VAL | A | 246 | −23.364 | 106.425 | −84.860 | 1.00 | 33.87 | A |
| ATOM | 804 | CG1 | VAL | A | 246 | −22.924 | 107.607 | −84.069 | 1.00 | 34.38 | A |
| ATOM | 805 | CG2 | VAL | A | 246 | −22.160 | 105.728 | −85.426 | 1.00 | 34.12 | A |
| ATOM | 806 | C | VAL | A | 246 | −25.471 | 106.166 | −83.586 | 1.00 | 34.38 | A |
| ATOM | 807 | O | VAL | A | 246 | −26.060 | 106.891 | −84.381 | 1.00 | 33.67 | A |
| ATOM | 808 | N | PHE | A | 247 | −25.910 | 105.942 | −82.354 | 1.00 | 34.78 | A |
| ATOM | 809 | CA | PHE | A | 247 | −27.119 | 106.553 | −81.849 | 1.00 | 32.44 | A |
| ATOM | 810 | CB | PHE | A | 247 | −27.241 | 106.295 | −80.351 | 1.00 | 31.96 | A |
| ATOM | 811 | CG | PHE | A | 247 | −27.582 | 104.878 | −79.995 | 1.00 | 32.09 | A |
| ATOM | 812 | CD1 | PHE | A | 247 | −28.481 | 104.149 | −80.761 | 1.00 | 31.38 | A |
| ATOM | 813 | CD2 | PHE | A | 247 | −27.068 | 104.298 | −78.840 | 1.00 | 33.87 | A |
| ATOM | 814 | CE1 | PHE | A | 247 | −28.865 | 102.867 | −80.378 | 1.00 | 32.81 | A |
| ATOM | 815 | CE2 | PHE | A | 247 | −27.448 | 103.015 | −78.453 | 1.00 | 33.44 | A |
| ATOM | 816 | CZ | PHE | A | 247 | −28.350 | 102.301 | −79.224 | 1.00 | 32.59 | A |
| ATOM | 817 | C | PHE | A | 247 | −27.012 | 108.049 | −82.097 | 1.00 | 32.71 | A |
| ATOM | 818 | O | PHE | A | 247 | −25.931 | 108.557 | −82.393 | 1.00 | 30.31 | A |
| ATOM | 819 | N | THR | A | 248 | −28.132 | 108.751 | −81.970 | 1.00 | 34.47 | A |
| ATOM | 820 | CA | THR | A | 248 | −28.166 | 110.199 | −82.170 | 1.00 | 35.62 | A |
| ATOM | 821 | CB | THR | A | 248 | −29.602 | 110.694 | −82.415 | 1.00 | 38.60 | A |
| ATOM | 822 | OG1 | THR | A | 248 | −30.311 | 109.742 | −83.225 | 1.00 | 40.72 | A |
| ATOM | 823 | CG2 | THR | A | 248 | −29.578 | 112.050 | −83.110 | 1.00 | 37.55 | A |
| ATOM | 824 | C | THR | A | 248 | −27.661 | 110.887 | −80.911 | 1.00 | 34.79 | A |
| ATOM | 825 | O | THR | A | 248 | −27.903 | 110.410 | −79.803 | 1.00 | 34.05 | A |
| ATOM | 826 | N | GLU | A | 249 | −26.972 | 112.008 | −81.081 | 1.00 | 34.51 | A |
| ATOM | 827 | CA | GLU | A | 249 | −26.449 | 112.760 | −79.944 | 1.00 | 35.98 | A |
| ATOM | 828 | CB | GLU | A | 249 | −25.923 | 114.117 | −80.405 | 1.00 | 34.13 | A |
| ATOM | 829 | CG | GLU | A | 249 | −24.432 | 114.183 | −80.652 | 1.00 | 31.70 | A |
| ATOM | 830 | CD | GLU | A | 249 | −23.931 | 115.615 | −80.719 | 1.00 | 30.48 | A |
| ATOM | 831 | OE1 | GLU | A | 249 | −24.105 | 116.350 | −79.724 | 1.00 | 26.58 | A |
| ATOM | 832 | OE2 | GLU | A | 249 | −23.366 | 116.000 | −81.763 | 1.00 | 27.76 | A |
| ATOM | 833 | C | GLU | A | 249 | −27.524 | 112.972 | −78.870 | 1.00 | 39.20 | A |
| ATOM | 834 | O | GLU | A | 249 | −27.241 | 113.423 | −77.755 | 1.00 | 39.80 | A |
| ATOM | 835 | N | GLU | A | 250 | −28.759 | 112.645 | −79.231 | 1.00 | 41.58 | A |
| ATOM | 836 | CA | GLU | A | 250 | −29.912 | 112.753 | −78.352 | 1.00 | 43.56 | A |
| ATOM | 837 | CB | GLU | A | 250 | −31.063 | 113.352 | −79.116 | 1.00 | 45.82 | A |
| ATOM | 838 | CG | GLU | A | 250 | −30.930 | 114.817 | −79.244 | 1.00 | 48.09 | A |
| ATOM | 839 | CD | GLU | A | 250 | −31.169 | 115.469 | −77.926 | 1.00 | 47.68 | A |
| ATOM | 840 | OE1 | GLU | A | 250 | −32.320 | 115.377 | −77.455 | 1.00 | 46.05 | A |
| ATOM | 841 | OE2 | GLU | A | 250 | −30.209 | 116.041 | −77.365 | 1.00 | 47.24 | A |
| ATOM | 842 | C | GLU | A | 250 | −30.278 | 111.364 | −77.915 | 1.00 | 44.41 | A |
| ATOM | 843 | O | GLU | A | 250 | −30.681 | 111.135 | −76.779 | 1.00 | 45.34 | A |
| ATOM | 844 | N | ARG | A | 251 | −30.134 | 110.442 | −78.858 | 1.00 | 45.42 | A |
| ATOM | 845 | CA | ARG | A | 251 | −30.394 | 109.027 | −78.643 | 1.00 | 45.39 | A |
| ATOM | 846 | CB | ARG | A | 251 | −30.031 | 108.252 | −79.891 | 1.00 | 44.94 | A |
| ATOM | 847 | CG | ARG | A | 251 | −31.130 | 107.370 | −80.395 | 1.00 | 45.93 | A |
| ATOM | 848 | CD | ARG | A | 251 | −31.338 | 106.185 | −79.512 | 1.00 | 43.02 | A |
| ATOM | 849 | NE | ARG | A | 251 | −31.711 | 105.064 | −80.352 | 1.00 | 45.83 | A |
| ATOM | 850 | CZ | ARG | A | 251 | −31.936 | 103.834 | −79.910 | 1.00 | 48.35 | A |
| ATOM | 851 | NH1 | ARG | A | 251 | −31.825 | 103.562 | −78.619 | 1.00 | 48.46 | A |
| ATOM | 852 | NH2 | ARG | A | 251 | −32.262 | 102.870 | −80.764 | 1.00 | 49.08 | A |
| ATOM | 853 | C | ARG | A | 251 | −29.514 | 108.568 | −77.497 | 1.00 | 44.99 | A |
| ATOM | 854 | O | ARG | A | 251 | −29.847 | 107.624 | −76.782 | 1.00 | 45.06 | A |
| ATOM | 855 | N | ALA | A | 252 | −28.368 | 109.229 | −77.350 | 1.00 | 42.22 | A |
| ATOM | 856 | CA | ALA | A | 252 | −27.462 | 108.928 | −76.263 | 1.00 | 40.76 | A |
| ATOM | 857 | CB | ALA | A | 252 | −26.030 | 109.281 | −76.648 | 1.00 | 39.88 | A |
| ATOM | 858 | C | ALA | A | 252 | −27.946 | 109.809 | −75.123 | 1.00 | 40.70 | A |
| ATOM | 859 | O | ALA | A | 252 | −28.134 | 109.344 | −74.008 | 1.00 | 41.22 | A |
| ATOM | 860 | N | ARG | A | 253 | −28.167 | 111.085 | −75.429 | 1.00 | 40.48 | A |
| ATOM | 861 | CA | ARG | A | 253 | −28.637 | 112.057 | −74.453 | 1.00 | 38.59 | A |
| ATOM | 862 | CB | ARG | A | 253 | −29.344 | 113.219 | −75.173 | 1.00 | 37.81 | A |
| ATOM | 863 | CG | ARG | A | 253 | −29.896 | 114.356 | −74.281 | 1.00 | 34.71 | A |
| ATOM | 864 | CD | ARG | A | 253 | −28.965 | 115.572 | −74.226 | 1.00 | 30.13 | A |
| ATOM | 865 | NE | ARG | A | 253 | −28.727 | 116.108 | −75.559 | 1.00 | 29.06 | A |
| ATOM | 866 | CZ | ARG | A | 253 | −27.731 | 116.928 | −75.880 | 1.00 | 29.79 | A |
| ATOM | 867 | NH1 | ARG | A | 253 | −26.879 | 117.325 | −74.955 | 1.00 | 25.42 | A |
| ATOM | 868 | NH2 | ARG | A | 253 | −27.551 | 117.304 | −77.142 | 1.00 | 27.32 | A |
| ATOM | 869 | C | ARG | A | 253 | −29.598 | 111.377 | −73.482 | 1.00 | 38.51 | A |
| ATOM | 870 | O | ARG | A | 253 | −29.452 | 111.502 | −72.263 | 1.00 | 38.00 | A |
| ATOM | 871 | N | PHE | A | 254 | −30.563 | 110.640 | −74.018 | 1.00 | 37.52 | A |
| ATOM | 872 | CA | PHE | A | 254 | −31.544 | 109.961 | −73.170 | 1.00 | 38.28 | A |
| ATOM | 873 | CB | PHE | A | 254 | −32.545 | 109.183 | −74.021 | 1.00 | 40.43 | A |
| ATOM | 874 | CG | PHE | A | 254 | −33.726 | 108.671 | −73.252 | 1.00 | 42.97 | A |
| ATOM | 875 | CD1 | PHE | A | 254 | −34.612 | 109.557 | −72.646 | 1.00 | 44.15 | A |
| ATOM | 876 | CD2 | PHE | A | 254 | −33.980 | 107.308 | −73.169 | 1.00 | 44.39 | A |
| ATOM | 877 | CE1 | PHE | A | 254 | −35.739 | 109.096 | −71.969 | 1.00 | 44.57 | A |
| ATOM | 878 | CE2 | PHE | A | 254 | −35.105 | 106.834 | −72.494 | 1.00 | 46.74 | A |
| ATOM | 879 | CZ | PHE | A | 254 | −35.991 | 107.734 | −71.894 | 1.00 | 46.36 | A |
| ATOM | 880 | C | PHE | A | 254 | −30.876 | 109.007 | −72.197 | 1.00 | 37.59 | A |
| ATOM | 881 | O | PHE | A | 254 | −30.984 | 109.172 | −70.983 | 1.00 | 38.31 | A |
| ATOM | 882 | N | TYR | A | 255 | −30.187 | 108.009 | −72.744 | 1.00 | 36.49 | A |
| ATOM | 883 | CA | TYR | A | 255 | −29.481 | 107.008 | −71.955 | 1.00 | 34.51 | A |
| ATOM | 884 | CB | TYR | A | 255 | −28.764 | 106.031 | −72.878 | 1.00 | 34.38 | A |
| ATOM | 885 | CG | TYR | A | 255 | −29.697 | 105.399 | −73.867 | 1.00 | 36.38 | A |
| ATOM | 886 | CD1 | TYR | A | 255 | −30.861 | 104.782 | −73.440 | 1.00 | 36.86 | A |
| ATOM | 887 | CE1 | TYR | A | 255 | −31.754 | 104.249 | −74.341 | 1.00 | 37.89 | A |
| ATOM | 888 | CD2 | TYR | A | 255 | −29.445 | 105.457 | −75.231 | 1.00 | 36.21 | A |
| ATOM | 889 | CE2 | TYR | A | 255 | −30.336 | 104.922 | −76.143 | 1.00 | 37.02 | A |
| ATOM | 890 | CZ | TYR | A | 255 | −31.489 | 104.326 | −75.691 | 1.00 | 36.90 | A |
| ATOM | 891 | OH | TYR | A | 255 | −32.414 | 103.838 | −76.571 | 1.00 | 37.31 | A |
| ATOM | 892 | C | TYR | A | 255 | −28.470 | 107.639 | −71.025 | 1.00 | 33.28 | A |
| ATOM | 893 | O | TYR | A | 255 | −28.510 | 107.436 | −69.821 | 1.00 | 33.03 | A |
| ATOM | 894 | N | GLY | A | 256 | −27.560 | 108.404 | −71.596 | 1.00 | 32.52 | A |
| ATOM | 895 | CA | GLY | A | 256 | −26.547 | 109.041 | −70.792 | 1.00 | 33.15 | A |
| ATOM | 896 | C | GLY | A | 256 | −27.176 | 109.791 | −69.652 | 1.00 | 33.67 | A |
| ATOM | 897 | O | GLY | A | 256 | −26.544 | 110.007 | −68.619 | 1.00 | 34.79 | A |
| ATOM | 898 | N | ALA | A | 257 | −28.424 | 110.199 | −69.835 | 1.00 | 32.91 | A |
| ATOM | 899 | CA | ALA | A | 257 | −29.116 | 110.919 | −68.781 | 1.00 | 33.05 | A |
| ATOM | 900 | CB | ALA | A | 257 | −30.291 | 111.691 | −69.347 | 1.00 | 32.88 | A |
| ATOM | 901 | C | ALA | A | 257 | −29.585 | 109.942 | −67.711 | 1.00 | 31.96 | A |
| ATOM | 902 | O | ALA | A | 257 | −29.381 | 110.159 | −66.529 | 1.00 | 31.69 | A |
| ATOM | 903 | N | GLU | A | 258 | −30.207 | 108.855 | −68.126 | 1.00 | 30.83 | A |
| ATOM | 904 | CA | GLU | A | 258 | −30.673 | 107.880 | −67.164 | 1.00 | 32.74 | A |
| ATOM | 905 | CB | GLU | A | 258 | −31.424 | 106.772 | −67.888 | 1.00 | 32.87 | A |
| ATOM | 906 | CG | GLU | A | 258 | −32.516 | 107.302 | −68.790 | 1.00 | 34.21 | A |
| ATOM | 907 | CD | GLU | A | 258 | −33.530 | 106.249 | −69.116 | 1.00 | 35.49 | A |
| ATOM | 908 | OE1 | GLU | A | 258 | −33.111 | 105.169 | −69.560 | 1.00 | 36.86 | A |
| ATOM | 909 | OE2 | GLU | A | 258 | −34.739 | 106.497 | −68.926 | 1.00 | 34.07 | A |
| ATOM | 910 | C | GLU | A | 258 | −29.539 | 107.291 | −66.319 | 1.00 | 33.81 | A |
| ATOM | 911 | O | GLU | A | 258 | −29.720 | 107.023 | −65.129 | 1.00 | 34.65 | A |
| ATOM | 912 | N | ILE | A | 259 | −28.371 | 107.105 | −66.925 | 1.00 | 33.33 | A |
| ATOM | 913 | CA | ILE | A | 259 | −27.229 | 106.552 | −66.207 | 1.00 | 32.69 | A |
| ATOM | 914 | CB | ILE | A | 259 | −26.091 | 106.159 | −67.168 | 1.00 | 32.27 | A |
| ATOM | 915 | CG2 | ILE | A | 259 | −24.926 | 105.569 | −66.378 | 1.00 | 31.18 | A |
| ATOM | 916 | CG1 | ILE | A | 259 | −26.602 | 105.158 | −68.200 | 1.00 | 29.40 | A |
| ATOM | 917 | CD1 | ILE | A | 259 | −25.626 | 104.896 | −69.254 | 1.00 | 29.61 | A |
| ATOM | 918 | C | ILE | A | 259 | −26.671 | 107.532 | −65.183 | 1.00 | 32.05 | A |
| ATOM | 919 | O | ILE | A | 259 | −26.199 | 107.126 | −64.121 | 1.00 | 31.97 | A |
| ATOM | 920 | N | VAL | A | 260 | −26.711 | 108.818 | −65.523 | 1.00 | 32.02 | A |
| ATOM | 921 | CA | VAL | A | 260 | −26.229 | 109.888 | −64.631 | 1.00 | 32.08 | A |
| ATOM | 922 | CB | VAL | A | 260 | −26.093 | 111.249 | −65.384 | 1.00 | 29.83 | A |
| ATOM | 923 | CG1 | VAL | A | 260 | −25.724 | 112.352 | −64.411 | 1.00 | 25.00 | A |
| ATOM | 924 | CG2 | VAL | A | 260 | −25.056 | 111.139 | −66.485 | 1.00 | 28.32 | A |
| ATOM | 925 | C | VAL | A | 260 | −27.179 | 110.096 | −63.439 | 1.00 | 31.55 | A |
| ATOM | 926 | O | VAL | A | 260 | −26.763 | 110.558 | −62.389 | 1.00 | 32.28 | A |
| ATOM | 927 | N | SER | A | 261 | −28.451 | 109.757 | −63.612 | 1.00 | 29.94 | A |
| ATOM | 928 | CA | SER | A | 261 | −29.424 | 109.900 | −62.545 | 1.00 | 30.84 | A |
| ATOM | 929 | CB | SER | A | 261 | −30.833 | 109.632 | −63.084 | 1.00 | 31.81 | A |
| ATOM | 930 | OG | SER | A | 261 | −31.828 | 110.009 | −62.151 | 1.00 | 30.44 | A |
| ATOM | 931 | C | SER | A | 261 | −29.079 | 108.858 | −61.487 | 1.00 | 31.10 | A |
| ATOM | 932 | O | SER | A | 261 | −29.088 | 109.130 | −60.291 | 1.00 | 31.07 | A |
| ATOM | 933 | N | ALA | A | 262 | −28.767 | 107.657 | −61.964 | 1.00 | 30.84 | A |
| ATOM | 934 | CA | ALA | A | 262 | −28.416 | 106.533 | −61.112 | 1.00 | 27.97 | A |
| ATOM | 935 | CB | ALA | A | 262 | −28.404 | 105.262 | −61.935 | 1.00 | 28.46 | A |
| ATOM | 936 | C | ALA | A | 262 | −27.078 | 106.707 | −60.415 | 1.00 | 25.94 | A |
| ATOM | 937 | O | ALA | A | 262 | −26.823 | 106.079 | −59.405 | 1.00 | 26.90 | A |
| ATOM | 938 | N | LEU | A | 263 | −26.227 | 107.564 | −60.949 | 1.00 | 23.62 | A |
| ATOM | 939 | CA | LEU | A | 263 | −24.919 | 107.773 | −60.366 | 1.00 | 24.02 | A |
| ATOM | 940 | CB | LEU | A | 263 | −23.887 | 108.048 | −61.452 | 1.00 | 23.45 | A |
| ATOM | 941 | CG | LEU | A | 263 | −23.570 | 106.878 | −62.377 | 1.00 | 22.05 | A |
| ATOM | 942 | CD1 | LEU | A | 263 | −22.324 | 107.208 | −63.170 | 1.00 | 21.25 | A |
| ATOM | 943 | CD2 | LEU | A | 263 | −23.388 | 105.621 | −61.558 | 1.00 | 20.13 | A |
| ATOM | 944 | C | LEU | A | 263 | −24.846 | 108.865 | −59.336 | 1.00 | 26.44 | A |
| ATOM | 945 | O | LEU | A | 263 | −23.991 | 108.814 | −58.460 | 1.00 | 26.99 | A |
| ATOM | 946 | N | GLU | A | 264 | −25.709 | 109.873 | −59.453 | 1.00 | 28.99 | A |
| ATOM | 947 | CA | GLU | A | 264 | −25.732 | 110.948 | −58.462 | 1.00 | 28.77 | A |
| ATOM | 948 | CB | GLU | A | 264 | −26.614 | 112.103 | −58.931 | 1.00 | 28.24 | A |
| ATOM | 949 | CG | GLU | A | 264 | −26.605 | 113.264 | −57.955 | 1.00 | 31.63 | A |
| ATOM | 950 | CD | GLU | A | 264 | −27.616 | 114.338 | −58.274 | 1.00 | 32.30 | A |
| ATOM | 951 | OE1 | GLU | A | 264 | −28.801 | 113.996 | −58.454 | 1.00 | 32.81 | A |
| ATOM | 952 | OE2 | GLU | A | 264 | −27.225 | 115.524 | −58.322 | 1.00 | 33.53 | A |
| ATOM | 953 | C | GLU | A | 264 | −26.336 | 110.291 | −57.215 | 1.00 | 28.46 | A |
| ATOM | 954 | O | GLU | A | 264 | −25.952 | 110.568 | −56.079 | 1.00 | 26.27 | A |
| ATOM | 955 | N | TYR | A | 265 | −27.271 | 109.384 | −57.479 | 1.00 | 27.85 | A |
| ATOM | 956 | CA | TYR | A | 265 | −27.963 | 108.612 | −56.463 | 1.00 | 29.34 | A |
| ATOM | 957 | CB | TYR | A | 265 | −29.099 | 107.820 | −57.100 | 1.00 | 29.72 | A |
| ATOM | 958 | CG | TYR | A | 265 | −29.598 | 106.710 | −56.216 | 1.00 | 33.41 | A |
| ATOM | 959 | CD1 | TYR | A | 265 | −30.425 | 106.970 | −55.129 | 1.00 | 34.71 | A |
| ATOM | 960 | CE1 | TYR | A | 265 | −30.841 | 105.945 | −54.286 | 1.00 | 35.99 | A |
| ATOM | 961 | CD2 | TYR | A | 265 | −29.201 | 105.396 | −56.435 | 1.00 | 37.60 | A |
| ATOM | 962 | CE2 | TYR | A | 265 | −29.614 | 104.362 | −55.591 | 1.00 | 37.88 | A |
| ATOM | 963 | CZ | TYR | A | 265 | −30.429 | 104.648 | −54.526 | 1.00 | 37.05 | A |
| ATOM | 964 | OH | TYR | A | 265 | −30.823 | 103.630 | −53.703 | 1.00 | 39.24 | A |
| ATOM | 965 | C | TYR | A | 265 | −27.035 | 107.635 | −55.743 | 1.00 | 29.17 | A |
| ATOM | 966 | O | TYR | A | 265 | −27.024 | 107.578 | −54.505 | 1.00 | 29.69 | A |
| ATOM | 967 | N | LEU | A | 266 | −26.293 | 106.846 | −56.522 | 1.00 | 26.54 | A |
| ATOM | 968 | CA | LEU | A | 266 | −25.369 | 105.862 | −55.966 | 1.00 | 24.17 | A |
| ATOM | 969 | CB | LEU | A | 266 | −24.856 | 104.908 | −57.038 | 1.00 | 22.32 | A |
| ATOM | 970 | CG | LEU | A | 266 | −25.724 | 103.728 | −57.431 | 1.00 | 22.72 | A |
| ATOM | 971 | CD1 | LEU | A | 266 | −25.048 | 102.968 | −58.546 | 1.00 | 20.84 | A |
| ATOM | 972 | CD2 | LEU | A | 266 | −25.954 | 102.847 | −56.231 | 1.00 | 22.60 | A |
| ATOM | 973 | C | LEU | A | 266 | −24.181 | 106.497 | −55.305 | 1.00 | 23.04 | A |
| ATOM | 974 | O | LEU | A | 266 | −23.651 | 105.963 | −54.357 | 1.00 | 21.67 | A |
| ATOM | 975 | N | HIS | A | 267 | −23.753 | 107.634 | −55.819 | 1.00 | 22.48 | A |
| ATOM | 976 | CA | HIS | A | 267 | −22.609 | 108.322 | −55.245 | 1.00 | 23.97 | A |
| ATOM | 977 | CB | HIS | A | 267 | −22.023 | 109.324 | −56.259 | 1.00 | 23.04 | A |
| ATOM | 978 | CG | HIS | A | 267 | −21.332 | 108.680 | −57.429 | 1.00 | 23.99 | A |
| ATOM | 979 | CD2 | HIS | A | 267 | −21.208 | 107.379 | −57.788 | 1.00 | 22.90 | A |
| ATOM | 980 | ND1 | HIS | A | 267 | −20.618 | 109.401 | −58.361 | 1.00 | 24.12 | A |
| ATOM | 981 | CE1 | HIS | A | 267 | −20.080 | 108.573 | −59.238 | 1.00 | 22.93 | A |
| ATOM | 982 | NE2 | HIS | A | 267 | −20.423 | 107.343 | −58.912 | 1.00 | 22.95 | A |
| ATOM | 983 | C | HIS | A | 267 | −22.973 | 109.042 | −53.951 | 1.00 | 24.92 | A |
| ATOM | 984 | O | HIS | A | 267 | −22.099 | 109.497 | −53.234 | 1.00 | 24.71 | A |
| ATOM | 985 | N | SER | A | 268 | −24.268 | 109.153 | −53.663 | 1.00 | 28.48 | A |
| ATOM | 986 | CA | SER | A | 268 | −24.722 | 109.840 | −52.462 | 1.00 | 30.48 | A |
| ATOM | 987 | CB | SER | A | 268 | −26.092 | 110.533 | −52.674 | 1.00 | 33.19 | A |
| ATOM | 988 | OG | SER | A | 268 | −27.135 | 109.656 | −53.107 | 1.00 | 36.18 | A |
| ATOM | 989 | C | SER | A | 268 | −24.784 | 108.852 | −51.331 | 1.00 | 30.30 | A |
| ATOM | 990 | O | SER | A | 268 | −24.966 | 109.222 | −50.182 | 1.00 | 30.83 | A |
| ATOM | 991 | N | ARG | A | 269 | −24.629 | 107.583 | −51.663 | 1.00 | 31.41 | A |
| ATOM | 992 | CA | ARG | A | 269 | −24.609 | 106.546 | −50.644 | 1.00 | 34.82 | A |
| ATOM | 993 | CB | ARG | A | 269 | −25.417 | 105.312 | −51.075 | 1.00 | 36.65 | A |
| ATOM | 994 | CG | ARG | A | 269 | −26.912 | 105.562 | −51.284 | 1.00 | 40.81 | A |
| ATOM | 995 | CD | ARG | A | 269 | −27.670 | 104.245 | −51.506 | 1.00 | 43.17 | A |
| ATOM | 996 | NE | ARG | A | 269 | −29.030 | 104.277 | −50.952 | 1.00 | 46.03 | A |
| ATOM | 997 | CZ | ARG | A | 269 | −29.900 | 103.270 | −51.031 | 1.00 | 46.64 | A |
| ATOM | 998 | NH1 | ARG | A | 269 | −29.554 | 102.139 | −51.640 | 1.00 | 47.74 | A |
| ATOM | 999 | NH2 | ARG | A | 269 | −31.123 | 103.400 | −50.527 | 1.00 | 45.34 | A |
| ATOM | 1000 | C | ARG | A | 269 | −23.149 | 106.145 | −50.432 | 1.00 | 35.15 | A |
| ATOM | 1001 | O | ARG | A | 269 | −22.853 | 105.332 | −49.561 | 1.00 | 37.64 | A |
| ATOM | 1002 | N | ASP | A | 270 | −22.246 | 106.730 | −51.223 | 1.00 | 33.55 | A |
| ATOM | 1003 | CA | ASP | A | 270 | −20.821 | 106.422 | −51.153 | 1.00 | 32.07 | A |
| ATOM | 1004 | CB | ASP | A | 270 | −20.355 | 106.364 | −49.702 | 1.00 | 34.09 | A |
| ATOM | 1005 | CG | ASP | A | 270 | −19.942 | 107.706 | −49.187 | 1.00 | 38.64 | A |
| ATOM | 1006 | OD1 | ASP | A | 270 | −18.759 | 108.086 | −49.337 | 1.00 | 39.23 | A |
| ATOM | 1007 | OD2 | ASP | A | 270 | −20.814 | 108.403 | −48.642 | 1.00 | 44.04 | A |
| ATOM | 1008 | C | ASP | A | 270 | −20.539 | 105.091 | −51.838 | 1.00 | 31.42 | A |
| ATOM | 1009 | O | ASP | A | 270 | −19.536 | 104.430 | −51.561 | 1.00 | 31.81 | A |
| ATOM | 1010 | N | VAL | A | 271 | −21.437 | 104.712 | −52.737 | 1.00 | 28.12 | A |
| ATOM | 1011 | CA | VAL | A | 271 | −21.330 | 103.472 | −53.483 | 1.00 | 25.52 | A |
| ATOM | 1012 | CB | VAL | A | 271 | −22.697 | 102.855 | −53.702 | 1.00 | 24.60 | A |
| ATOM | 1013 | CG1 | VAL | A | 271 | −22.565 | 101.626 | −54.554 | 1.00 | 21.84 | A |
| ATOM | 1014 | CG2 | VAL | A | 271 | −23.357 | 102.562 | −52.375 | 1.00 | 23.57 | A |
| ATOM | 1015 | C | VAL | A | 271 | −20.732 | 103.689 | −54.859 | 1.00 | 25.86 | A |
| ATOM | 1016 | O | VAL | A | 271 | −21.446 | 104.006 | −55.786 | 1.00 | 27.74 | A |
| ATOM | 1017 | N | VAL | A | 272 | −19.423 | 103.536 | −55.006 | 1.00 | 26.27 | A |
| ATOM | 1018 | CA | VAL | A | 272 | −18.828 | 103.707 | −56.324 | 1.00 | 25.25 | A |
| ATOM | 1019 | CB | VAL | A | 272 | −17.305 | 103.688 | −56.268 | 1.00 | 24.89 | A |
| ATOM | 1020 | CG1 | VAL | A | 272 | −16.739 | 103.513 | −57.665 | 1.00 | 23.43 | A |
| ATOM | 1021 | CG2 | VAL | A | 272 | −16.807 | 104.980 | −55.637 | 1.00 | 19.68 | A |
| ATOM | 1022 | C | VAL | A | 272 | −19.328 | 102.512 | −57.084 | 1.00 | 25.49 | A |
| ATOM | 1023 | O | VAL | A | 272 | −19.458 | 101.451 | −56.502 | 1.00 | 27.02 | A |
| ATOM | 1024 | N | TYR | A | 273 | −19.631 | 102.671 | −58.367 | 1.00 | 26.39 | A |
| ATOM | 1025 | CA | TYR | A | 273 | −20.154 | 101.542 | −59.138 | 1.00 | 25.65 | A |
| ATOM | 1026 | CB | TYR | A | 273 | −21.158 | 102.026 | −60.188 | 1.00 | 21.46 | A |
| ATOM | 1027 | CG | TYR | A | 273 | −22.014 | 100.907 | −60.728 | 1.00 | 19.34 | A |
| ATOM | 1028 | CD1 | TYR | A | 273 | −22.989 | 100.320 | −59.945 | 1.00 | 15.37 | A |
| ATOM | 1029 | CE1 | TYR | A | 273 | −23.734 | 99.269 | −60.414 | 1.00 | 15.86 | A |
| ATOM | 1030 | CD2 | TYR | A | 273 | −21.814 | 100.407 | −62.008 | 1.00 | 19.42 | A |
| ATOM | 1031 | CE2 | TYR | A | 273 | −22.565 | 99.351 | −62.490 | 1.00 | 17.46 | A |
| ATOM | 1032 | CZ | TYR | A | 273 | −23.518 | 98.786 | −61.688 | 1.00 | 17.18 | A |
| ATOM | 1033 | OH | TYR | A | 273 | −24.255 | 97.719 | −62.147 | 1.00 | 20.93 | A |
| ATOM | 1034 | C | TYR | A | 273 | −19.097 | 100.658 | −59.804 | 1.00 | 25.88 | A |
| ATOM | 1035 | O | TYR | A | 273 | −19.187 | 99.444 | −59.731 | 1.00 | 25.15 | A |
| ATOM | 1036 | N | ARG | A | 274 | −18.114 | 101.264 | −60.460 | 1.00 | 27.43 | A |
| ATOM | 1037 | CA | ARG | A | 274 | −17.043 | 100.507 | −61.114 | 1.00 | 31.42 | A |
| ATOM | 1038 | CB | ARG | A | 274 | −16.427 | 99.499 | −60.129 | 1.00 | 31.33 | A |
| ATOM | 1039 | CG | ARG | A | 274 | −15.718 | 100.127 | −58.964 | 1.00 | 35.00 | A |
| ATOM | 1040 | CD | ARG | A | 274 | −15.394 | 99.118 | −57.887 | 1.00 | 36.47 | A |
| ATOM | 1041 | NE | ARG | A | 274 | −14.480 | 98.077 | −58.343 | 1.00 | 38.77 | A |
| ATOM | 1042 | CZ | ARG | A | 274 | −13.792 | 97.278 | −57.530 | 1.00 | 39.74 | A |
| ATOM | 1043 | NH1 | ARG | A | 274 | −13.908 | 97.404 | −56.213 | 1.00 | 40.96 | A |
| ATOM | 1044 | NH2 | ARG | A | 274 | −12.995 | 96.344 | −58.032 | 1.00 | 39.62 | A |
| ATOM | 1045 | C | ARG | A | 274 | −17.391 | 99.751 | −62.406 | 1.00 | 31.91 | A |
| ATOM | 1046 | O | ARG | A | 274 | −16.522 | 99.520 | −63.233 | 1.00 | 32.08 | A |
| ATOM | 1047 | N | ASP | A | 275 | −18.646 | 99.367 | −62.586 | 1.00 | 33.64 | A |
| ATOM | 1048 | CA | ASP | A | 275 | −19.000 | 98.608 | −63.769 | 1.00 | 35.02 | A |
| ATOM | 1049 | CB | ASP | A | 275 | −19.600 | 97.268 | −63.353 | 1.00 | 37.76 | A |
| ATOM | 1050 | CG | ASP | A | 275 | −18.573 | 96.347 | −62.731 | 1.00 | 40.90 | A |
| ATOM | 1051 | OD1 | ASP | A | 275 | −18.009 | 96.702 | −61.675 | 1.00 | 40.31 | A |
| ATOM | 1052 | OD2 | ASP | A | 275 | −18.328 | 95.265 | −63.305 | 1.00 | 44.05 | A |
| ATOM | 1053 | C | ASP | A | 275 | −19.895 | 99.263 | −64.803 | 1.00 | 35.21 | A |
| ATOM | 1054 | O | ASP | A | 275 | −20.629 | 98.570 | −65.509 | 1.00 | 34.29 | A |
| ATOM | 1055 | N | ILE | A | 276 | −19.831 | 100.588 | −64.906 | 1.00 | 34.89 | A |
| ATOM | 1056 | CA | ILE | A | 276 | −20.630 | 101.285 | −65.900 | 1.00 | 35.45 | A |
| ATOM | 1057 | CB | ILE | A | 276 | −20.863 | 102.772 | −65.539 | 1.00 | 35.25 | A |
| ATOM | 1058 | CG2 | ILE | A | 276 | −21.467 | 103.525 | −66.746 | 1.00 | 32.67 | A |
| ATOM | 1059 | CG1 | ILE | A | 276 | −21.824 | 102.860 | −64.345 | 1.00 | 34.05 | A |
| ATOM | 1060 | CD1 | ILE | A | 276 | −22.086 | 104.228 | −63.910 | 1.00 | 33.07 | A |
| ATOM | 1061 | C | ILE | A | 276 | −19.893 | 101.179 | −67.217 | 1.00 | 36.58 | A |
| ATOM | 1062 | O | ILE | A | 276 | −18.872 | 101.828 | −67.432 | 1.00 | 35.65 | A |
| ATOM | 1063 | N | LYS | A | 277 | −20.434 | 100.325 | −68.082 | 1.00 | 37.98 | A |
| ATOM | 1064 | CA | LYS | A | 277 | −19.880 | 100.038 | −69.397 | 1.00 | 39.08 | A |
| ATOM | 1065 | CB | LYS | A | 277 | −18.713 | 99.046 | −69.263 | 1.00 | 38.92 | A |
| ATOM | 1066 | CG | LYS | A | 277 | −19.095 | 97.761 | −68.545 | 1.00 | 40.30 | A |
| ATOM | 1067 | CD | LYS | A | 277 | −17.986 | 96.729 | −68.543 | 1.00 | 41.04 | A |
| ATOM | 1068 | CE | LYS | A | 277 | −18.491 | 95.371 | −68.026 | 1.00 | 42.56 | A |
| ATOM | 1069 | NZ | LYS | A | 277 | −17.534 | 94.248 | −68.274 | 1.00 | 40.96 | A |
| ATOM | 1070 | C | LYS | A | 277 | −21.015 | 99.433 | −70.231 | 1.00 | 39.64 | A |
| ATOM | 1071 | O | LYS | A | 277 | −22.017 | 98.980 | −69.679 | 1.00 | 40.48 | A |
| ATOM | 1072 | N | LEU | A | 278 | −20.834 | 99.418 | −71.549 | 1.00 | 39.22 | A |
| ATOM | 1073 | CA | LEU | A | 278 | −21.822 | 98.940 | −72.519 | 1.00 | 38.63 | A |
| ATOM | 1074 | CB | LEU | A | 278 | −21.212 | 98.943 | −73.901 | 1.00 | 38.08 | A |
| ATOM | 1075 | CG | LEU | A | 278 | −21.805 | 99.976 | −74.820 | 1.00 | 37.92 | A |
| ATOM | 1076 | CD1 | LEU | A | 278 | −20.695 | 100.904 | −75.240 | 1.00 | 38.72 | A |
| ATOM | 1077 | CD2 | LEU | A | 278 | −22.462 | 99.305 | −75.995 | 1.00 | 37.44 | A |
| ATOM | 1078 | C | LEU | A | 278 | −22.538 | 97.622 | −72.370 | 1.00 | 39.89 | A |
| ATOM | 1079 | O | LEU | A | 278 | −23.660 | 97.470 | −72.879 | 1.00 | 39.13 | A |
| ATOM | 1080 | N | GLU | A | 279 | −21.890 | 96.648 | −71.736 | 1.00 | 41.04 | A |
| ATOM | 1081 | CA | GLU | A | 279 | −22.527 | 95.346 | −71.561 | 1.00 | 40.42 | A |
| ATOM | 1082 | CB | GLU | A | 279 | −21.476 | 94.246 | −71.473 | 1.00 | 42.80 | A |
| ATOM | 1083 | CG | GLU | A | 279 | −21.726 | 93.134 | −72.469 | 1.00 | 45.57 | A |
| ATOM | 1084 | CD | GLU | A | 279 | −22.036 | 93.680 | −73.858 | 1.00 | 47.03 | A |
| ATOM | 1085 | OE1 | GLU | A | 279 | −21.250 | 94.536 | −74.339 | 1.00 | 46.93 | A |
| ATOM | 1086 | OE2 | GLU | A | 279 | −23.054 | 93.254 | −74.458 | 1.00 | 44.67 | A |
| ATOM | 1087 | C | GLU | A | 279 | −23.410 | 95.357 | −70.330 | 1.00 | 37.93 | A |
| ATOM | 1088 | O | GLU | A | 279 | −24.316 | 94.552 | −70.183 | 1.00 | 37.80 | A |
| ATOM | 1089 | N | ASN | A | 280 | −23.145 | 96.304 | −69.453 | 1.00 | 36.16 | A |
| ATOM | 1090 | CA | ASN | A | 280 | −23.936 | 96.443 | −68.255 | 1.00 | 36.11 | A |
| ATOM | 1091 | CB | ASN | A | 280 | −23.133 | 97.186 | −67.182 | 1.00 | 36.30 | A |
| ATOM | 1092 | CG | ASN | A | 280 | −22.218 | 96.269 | −66.404 | 1.00 | 34.57 | A |
| ATOM | 1093 | OD1 | ASN | A | 280 | −22.579 | 95.786 | −65.345 | 1.00 | 35.55 | A |
| ATOM | 1094 | ND2 | ASN | A | 280 | −21.039 | 96.014 | −66.936 | 1.00 | 33.27 | A |
| ATOM | 1095 | C | ASN | A | 280 | −25.222 | 97.190 | −68.562 | 1.00 | 34.76 | A |
| ATOM | 1096 | O | ASN | A | 280 | −26.061 | 97.338 | −67.696 | 1.00 | 34.70 | A |
| ATOM | 1097 | N | LEU | A | 281 | −25.375 | 97.636 | −69.805 | 1.00 | 35.35 | A |
| ATOM | 1098 | CA | LEU | A | 281 | −26.556 | 98.398 | −70.236 | 1.00 | 35.38 | A |
| ATOM | 1099 | CB | LEU | A | 281 | −26.106 | 99.657 | −70.991 | 1.00 | 34.23 | A |
| ATOM | 1100 | CG | LEU | A | 281 | −25.228 | 100.592 | −70.156 | 1.00 | 35.27 | A |
| ATOM | 1101 | CD1 | LEU | A | 281 | −24.698 | 101.766 | −70.982 | 1.00 | 35.67 | A |
| ATOM | 1102 | CD2 | LEU | A | 281 | −26.049 | 101.091 | −68.981 | 1.00 | 37.36 | A |
| ATOM | 1103 | C | LEU | A | 281 | −27.577 | 97.630 | −71.089 | 1.00 | 35.68 | A |
| ATOM | 1104 | O | LEU | A | 281 | −27.251 | 97.072 | −72.135 | 1.00 | 34.57 | A |
| ATOM | 1105 | N | MET | A | 282 | −28.816 | 97.603 | −70.611 | 1.00 | 36.34 | A |
| ATOM | 1106 | CA | MET | A | 282 | −29.921 | 96.940 | −71.298 | 1.00 | 37.12 | A |
| ATOM | 1107 | CB | MET | A | 282 | −30.408 | 95.722 | −70.509 | 1.00 | 37.66 | A |
| ATOM | 1108 | CG | MET | A | 282 | −29.473 | 94.530 | −70.524 | 1.00 | 39.07 | A |
| ATOM | 1109 | SD | MET | A | 282 | −30.340 | 92.945 | −70.189 | 1.00 | 42.02 | A |
| ATOM | 1110 | CE | MET | A | 282 | −30.039 | 92.718 | −68.422 | 1.00 | 40.51 | A |
| ATOM | 1111 | C | MET | A | 282 | −31.058 | 97.957 | −71.415 | 1.00 | 37.98 | A |
| ATOM | 1112 | O | MET | A | 282 | −30.856 | 99.145 | −71.141 | 1.00 | 37.86 | A |
| ATOM | 1113 | N | LEU | A | 283 | −32.242 | 97.492 | −71.815 | 1.00 | 37.15 | A |
| ATOM | 1114 | CA | LEU | A | 283 | −33.425 | 98.346 | −71.965 | 1.00 | 36.88 | A |
| ATOM | 1115 | CB | LEU | A | 283 | −33.565 | 98.838 | −73.412 | 1.00 | 32.01 | A |
| ATOM | 1116 | CG | LEU | A | 283 | −32.473 | 99.715 | −74.031 | 1.00 | 27.90 | A |
| ATOM | 1117 | CD1 | LEU | A | 283 | −32.773 | 99.914 | −75.493 | 1.00 | 27.78 | A |
| ATOM | 1118 | CD2 | LEU | A | 283 | −32.399 | 101.042 | −73.334 | 1.00 | 25.20 | A |
| ATOM | 1119 | C | LEU | A | 283 | −34.663 | 97.535 | −71.584 | 1.00 | 39.75 | A |
| ATOM | 1120 | O | LEU | A | 283 | −34.795 | 96.380 | −71.991 | 1.00 | 39.81 | A |
| ATOM | 1121 | N | ASP | A | 284 | −35.570 | 98.128 | −70.806 | 1.00 | 41.87 | A |
| ATOM | 1122 | CA | ASP | A | 284 | −36.772 | 97.415 | −70.391 | 1.00 | 43.84 | A |
| ATOM | 1123 | CB | ASP | A | 284 | −37.252 | 97.893 | −69.012 | 1.00 | 46.24 | A |
| ATOM | 1124 | CG | ASP | A | 284 | −37.389 | 99.410 | −68.909 | 1.00 | 48.50 | A |
| ATOM | 1125 | OD1 | ASP | A | 284 | −37.751 | 100.054 | −69.917 | 1.00 | 46.51 | A |
| ATOM | 1126 | OD2 | ASP | A | 284 | −37.152 | 99.945 | −67.796 | 1.00 | 48.37 | A |
| ATOM | 1127 | C | ASP | A | 284 | −37.897 | 97.539 | −71.403 | 1.00 | 45.15 | A |
| ATOM | 1128 | O | ASP | A | 284 | −37.818 | 98.344 | −72.327 | 1.00 | 44.64 | A |
| ATOM | 1129 | N | LYS | A | 285 | −38.940 | 96.733 | −71.224 | 1.00 | 46.02 | A |
| ATOM | 1130 | CA | LYS | A | 285 | −40.086 | 96.734 | −72.132 | 1.00 | 46.07 | A |
| ATOM | 1131 | CB | LYS | A | 285 | −41.341 | 96.168 | −71.436 | 1.00 | 45.21 | A |
| ATOM | 1132 | CG | LYS | A | 285 | −41.546 | 94.643 | −71.546 | 1.00 | 45.28 | A |
| ATOM | 1133 | CD | LYS | A | 285 | −42.992 | 94.262 | −71.158 | 1.00 | 44.25 | A |
| ATOM | 1134 | CE | LYS | A | 285 | −43.288 | 92.751 | −71.190 | 1.00 | 41.92 | A |
| ATOM | 1135 | NZ | LYS | A | 285 | −42.787 | 92.018 | −69.994 | 1.00 | 39.57 | A |
| ATOM | 1136 | C | LYS | A | 285 | −40.430 | 98.087 | −72.753 | 1.00 | 45.07 | A |
| ATOM | 1137 | O | LYS | A | 285 | −40.823 | 98.134 | −73.914 | 1.00 | 45.90 | A |
| ATOM | 1138 | N | ASP | A | 286 | −40.262 | 99.180 | −72.008 | 1.00 | 43.75 | A |
| ATOM | 1139 | CA | ASP | A | 286 | −40.609 | 100.516 | −72.517 | 1.00 | 42.99 | A |
| ATOM | 1140 | CB | ASP | A | 286 | −41.235 | 101.371 | −71.400 | 1.00 | 41.55 | A |
| ATOM | 1141 | CG | ASP | A | 286 | −42.373 | 100.665 | −70.677 | 1.00 | 42.05 | A |
| ATOM | 1142 | OD1 | ASP | A | 286 | −43.357 | 100.275 | −71.346 | 1.00 | 42.14 | A |
| ATOM | 1143 | OD2 | ASP | A | 286 | −42.285 | 100.500 | −69.432 | 1.00 | 42.02 | A |
| ATOM | 1144 | C | ASP | A | 286 | −39.520 | 101.357 | −73.187 | 1.00 | 42.82 | A |
| ATOM | 1145 | O | ASP | A | 286 | −39.827 | 102.405 | −73.745 | 1.00 | 44.03 | A |
| ATOM | 1146 | N | GLY | A | 287 | −38.262 | 100.929 | −73.127 | 1.00 | 43.61 | A |
| ATOM | 1147 | CA | GLY | A | 287 | −37.184 | 101.706 | −73.734 | 1.00 | 41.35 | A |
| ATOM | 1148 | C | GLY | A | 287 | −36.272 | 102.442 | −72.747 | 1.00 | 39.84 | A |
| ATOM | 1149 | O | GLY | A | 287 | −35.457 | 103.286 | −73.135 | 1.00 | 39.78 | A |
| ATOM | 1150 | N | HIS | A | 288 | −36.400 | 102.122 | −71.464 | 1.00 | 38.40 | A |
| ATOM | 1151 | CA | HIS | A | 288 | −35.584 | 102.741 | −70.422 | 1.00 | 36.35 | A |
| ATOM | 1152 | CB | HIS | A | 288 | −36.444 | 102.987 | −69.184 | 1.00 | 35.85 | A |
| ATOM | 1153 | CG | HIS | A | 288 | −37.360 | 104.157 | −69.310 | 1.00 | 36.47 | A |
| ATOM | 1154 | CD2 | HIS | A | 288 | −38.708 | 104.229 | −69.385 | 1.00 | 36.42 | A |
| ATOM | 1155 | ND1 | HIS | A | 288 | −36.902 | 105.455 | −69.399 | 1.00 | 36.41 | A |
| ATOM | 1156 | CE1 | HIS | A | 288 | −37.929 | 106.272 | −69.524 | 1.00 | 37.52 | A |
| ATOM | 1157 | NE2 | HIS | A | 288 | −39.038 | 105.552 | −69.519 | 1.00 | 36.25 | A |
| ATOM | 1158 | C | HIS | A | 288 | −34.360 | 101.891 | −70.041 | 1.00 | 34.72 | A |
| ATOM | 1159 | O | HIS | A | 288 | −34.477 | 100.695 | −69.811 | 1.00 | 34.41 | A |
| ATOM | 1160 | N | ILE | A | 289 | −33.193 | 102.522 | −69.966 | 1.00 | 32.41 | A |
| ATOM | 1161 | CA | ILE | A | 289 | −31.955 | 101.829 | −69.610 | 1.00 | 31.73 | A |
| ATOM | 1162 | CB | ILE | A | 289 | −30.742 | 102.782 | −69.607 | 1.00 | 30.57 | A |
| ATOM | 1163 | CG2 | ILE | A | 289 | −30.056 | 102.742 | −68.229 | 1.00 | 28.30 | A |
| ATOM | 1164 | CG1 | ILE | A | 289 | −29.764 | 102.401 | −70.731 | 1.00 | 29.20 | A |
| ATOM | 1165 | CD1 | ILE | A | 289 | −28.489 | 103.216 | −70.756 | 1.00 | 26.28 | A |
| ATOM | 1166 | C | ILE | A | 289 | −32.018 | 101.214 | −68.227 | 1.00 | 31.91 | A |
| ATOM | 1167 | O | ILE | A | 289 | −32.737 | 101.702 | −67.359 | 1.00 | 33.58 | A |
| ATOM | 1168 | N | LYS | A | 290 | −31.229 | 100.159 | −68.036 | 1.00 | 31.19 | A |
| ATOM | 1169 | CA | LYS | A | 290 | −31.117 | 99.430 | −66.766 | 1.00 | 29.97 | A |
| ATOM | 1170 | CB | LYS | A | 290 | −31.891 | 98.123 | −66.807 | 1.00 | 27.48 | A |
| ATOM | 1171 | CG | LYS | A | 290 | −33.361 | 98.218 | −66.597 | 1.00 | 26.64 | A |
| ATOM | 1172 | CD | LYS | A | 290 | −33.736 | 97.567 | −65.294 | 1.00 | 25.08 | A |
| ATOM | 1173 | CE | LYS | A | 290 | −33.432 | 98.474 | −64.158 | 1.00 | 25.34 | A |
| ATOM | 1174 | NZ | LYS | A | 290 | −34.477 | 99.510 | −64.077 | 1.00 | 24.39 | A |
| ATOM | 1175 | C | LYS | A | 290 | −29.654 | 99.071 | −66.561 | 1.00 | 30.57 | A |
| ATOM | 1176 | O | LYS | A | 290 | −29.158 | 98.168 | −67.223 | 1.00 | 31.66 | A |
| ATOM | 1177 | N | ILE | A | 291 | −28.963 | 99.781 | −65.673 | 1.00 | 30.15 | A |
| ATOM | 1178 | CA | ILE | A | 291 | −27.571 | 99.498 | −65.392 | 1.00 | 28.64 | A |
| ATOM | 1179 | CB | ILE | A | 291 | −26.997 | 100.504 | −64.407 | 1.00 | 28.60 | A |
| ATOM | 1180 | CG2 | ILE | A | 291 | −25.578 | 100.110 | −64.034 | 1.00 | 28.92 | A |
| ATOM | 1181 | CG1 | ILE | A | 291 | −27.006 | 101.904 | −65.027 | 1.00 | 28.24 | A |
| ATOM | 1182 | CD1 | ILE | A | 291 | −26.860 | 103.033 | −64.008 | 1.00 | 26.43 | A |
| ATOM | 1183 | C | ILE | A | 291 | −27.678 | 98.147 | −64.741 | 1.00 | 30.07 | A |
| ATOM | 1184 | O | ILE | A | 291 | −28.471 | 97.965 | −63.846 | 1.00 | 30.32 | A |
| ATOM | 1185 | N | THR | A | 292 | −26.885 | 97.187 | −65.182 | 1.00 | 32.81 | A |
| ATOM | 1186 | CA | THR | A | 292 | −27.014 | 95.853 | −64.634 | 1.00 | 35.39 | A |
| ATOM | 1187 | CB | THR | A | 292 | −27.744 | 94.972 | −65.680 | 1.00 | 32.81 | A |
| ATOM | 1188 | OG1 | THR | A | 292 | −28.197 | 93.761 | −65.082 | 1.00 | 32.18 | A |
| ATOM | 1189 | CG2 | THR | A | 292 | −26.853 | 94.673 | −66.820 | 1.00 | 30.94 | A |
| ATOM | 1190 | C | THR | A | 292 | −25.708 | 95.200 | −64.159 | 1.00 | 39.46 | A |
| ATOM | 1191 | O | THR | A | 292 | −24.771 | 95.876 | −63.728 | 1.00 | 38.51 | A |
| ATOM | 1192 | N | ASP | A | 293 | −25.682 | 93.874 | −64.205 | 1.00 | 45.81 | A |
| ATOM | 1193 | CA | ASP | A | 293 | −24.532 | 93.068 | −63.798 | 1.00 | 51.59 | A |
| ATOM | 1194 | CB | ASP | A | 293 | −23.490 | 93.056 | −64.919 | 1.00 | 53.04 | A |
| ATOM | 1195 | CG | ASP | A | 293 | −22.571 | 91.843 | −64.855 | 1.00 | 54.41 | A |
| ATOM | 1196 | OD1 | ASP | A | 293 | −23.097 | 90.707 | −64.705 | 1.00 | 54.78 | A |
| ATOM | 1197 | OD2 | ASP | A | 293 | −21.334 | 92.036 | −64.970 | 1.00 | 53.19 | A |
| ATOM | 1198 | C | ASP | A | 293 | −23.898 | 93.549 | −62.501 | 1.00 | 54.71 | A |
| ATOM | 1199 | O | ASP | A | 293 | −23.116 | 94.502 | −62.498 | 1.00 | 55.17 | A |
| ATOM | 1200 | N | PHE | A | 294 | −24.243 | 92.878 | −61.403 | 1.00 | 58.22 | A |
| ATOM | 1201 | CA | PHE | A | 294 | −23.717 | 93.220 | −60.091 | 1.00 | 62.47 | A |
| ATOM | 1202 | CB | PHE | A | 294 | −24.815 | 93.107 | −59.036 | 1.00 | 64.72 | A |
| ATOM | 1203 | CG | PHE | A | 294 | −25.456 | 94.417 | −58.659 | 1.00 | 67.85 | A |
| ATOM | 1204 | CD1 | PHE | A | 294 | −26.323 | 95.070 | −59.531 | 1.00 | 68.79 | A |
| ATOM | 1205 | CD2 | PHE | A | 294 | −25.229 | 94.975 | −57.403 | 1.00 | 68.87 | A |
| ATOM | 1206 | CE1 | PHE | A | 294 | −26.962 | 96.254 | −59.154 | 1.00 | 69.30 | A |
| ATOM | 1207 | CE2 | PHE | A | 294 | −25.866 | 96.162 | −57.018 | 1.00 | 69.41 | A |
| ATOM | 1208 | CZ | PHE | A | 294 | −26.733 | 96.799 | −57.896 | 1.00 | 68.81 | A |
| ATOM | 1209 | C | PHE | A | 294 | −22.562 | 92.303 | −59.684 | 1.00 | 64.77 | A |
| ATOM | 1210 | O | PHE | A | 294 | −22.782 | 91.163 | −59.264 | 1.00 | 65.50 | A |
| ATOM | 1211 | N | GLY | A | 295 | −21.332 | 92.797 | −59.788 | 1.00 | 65.64 | A |
| ATOM | 1212 | CA | GLY | A | 295 | −20.202 | 91.979 | −59.399 | 1.00 | 66.77 | A |
| ATOM | 1213 | C | GLY | A | 295 | −19.081 | 92.748 | −58.732 | 1.00 | 68.04 | A |
| ATOM | 1214 | O | GLY | A | 295 | −18.553 | 92.326 | −57.706 | 1.00 | 67.55 | A |
| ATOM | 1215 | N | LEU | A | 296 | −18.722 | 93.887 | −59.315 | 1.00 | 69.24 | A |
| ATOM | 1216 | CA | LEU | A | 296 | −17.636 | 94.714 | −58.796 | 1.00 | 69.80 | A |
| ATOM | 1217 | CB | LEU | A | 296 | −16.683 | 95.040 | −59.941 | 1.00 | 68.99 | A |
| ATOM | 1218 | CG | LEU | A | 296 | −16.702 | 93.978 | −61.051 | 1.00 | 69.63 | A |
| ATOM | 1219 | CD1 | LEU | A | 296 | −15.744 | 94.376 | −62.158 | 1.00 | 69.49 | A |
| ATOM | 1220 | CD2 | LEU | A | 296 | −16.321 | 92.612 | −60.487 | 1.00 | 69.24 | A |
| ATOM | 1221 | C | LEU | A | 296 | −18.121 | 96.008 | −58.118 | 1.00 | 70.84 | A |
| ATOM | 1222 | O | LEU | A | 296 | −17.974 | 97.103 | −58.663 | 1.00 | 71.52 | A |
| ATOM | 1223 | N | CYS | A | 297 | −18.698 | 95.870 | −56.927 | 1.00 | 70.50 | A |
| ATOM | 1224 | CA | CYS | A | 297 | −19.182 | 97.014 | −56.166 | 1.00 | 70.35 | A |
| ATOM | 1225 | CB | CYS | A | 297 | −20.617 | 97.346 | −56.562 | 1.00 | 70.19 | A |
| ATOM | 1226 | SG | CYS | A | 297 | −20.722 | 98.245 | −58.113 | 1.00 | 70.43 | A |
| ATOM | 1227 | C | CYS | A | 297 | −19.100 | 96.805 | −54.653 | 1.00 | 70.28 | A |
| ATOM | 1228 | O | CYS | A | 297 | −18.912 | 95.692 | −54.166 | 1.00 | 70.03 | A |
| ATOM | 1229 | N | LYS | A | 298 | −19.261 | 97.894 | −53.913 | 1.00 | 70.13 | A |
| ATOM | 1230 | CA | LYS | A | 298 | −19.186 | 97.847 | −52.464 | 1.00 | 69.38 | A |
| ATOM | 1231 | CB | LYS | A | 298 | −17.792 | 97.391 | −52.041 | 1.00 | 68.44 | A |
| ATOM | 1232 | CG | LYS | A | 298 | −17.634 | 97.221 | −50.548 | 1.00 | 69.24 | A |
| ATOM | 1233 | CD | LYS | A | 298 | −16.175 | 97.100 | −50.148 | 1.00 | 68.56 | A |
| ATOM | 1234 | CE | LYS | A | 298 | −16.016 | 97.147 | −48.632 | 1.00 | 66.43 | A |
| ATOM | 1235 | NZ | LYS | A | 298 | −14.603 | 97.390 | −48.238 | 1.00 | 63.63 | A |
| ATOM | 1236 | C | LYS | A | 298 | −19.460 | 99.239 | −51.905 | 1.00 | 69.46 | A |
| ATOM | 1237 | O | LYS | A | 298 | −19.586 | 100.205 | −52.656 | 1.00 | 69.37 | A |
| ATOM | 1238 | N | GLU | A | 299 | −19.541 | 99.339 | −50.586 | 1.00 | 69.37 | A |
| ATOM | 1239 | CA | GLU | A | 299 | −19.781 | 100.617 | −49.935 | 1.00 | 70.45 | A |
| ATOM | 1240 | CB | GLU | A | 299 | −20.687 | 100.423 | −48.735 | 1.00 | 69.62 | A |
| ATOM | 1241 | CG | GLU | A | 299 | −20.036 | 99.732 | −47.552 | 1.00 | 67.24 | A |
| ATOM | 1242 | CD | GLU | A | 299 | −21.060 | 99.321 | −46.522 | 1.00 | 64.27 | A |
| ATOM | 1243 | OE1 | GLU | A | 299 | −20.764 | 99.388 | −45.313 | 1.00 | 62.87 | A |
| ATOM | 1244 | OE2 | GLU | A | 299 | −22.168 | 98.925 | −46.930 | 1.00 | 61.93 | A |
| ATOM | 1245 | C | GLU | A | 299 | −18.468 | 101.244 | −49.470 | 1.00 | 72.35 | A |
| ATOM | 1246 | O | GLU | A | 299 | −18.463 | 102.257 | −48.759 | 1.00 | 72.33 | A |
| ATOM | 1247 | N | VAL | A | 302 | −17.355 | 100.625 | −49.854 | 1.00 | 73.46 | A |
| ATOM | 1248 | CA | VAL | A | 302 | −16.046 | 101.143 | −49.489 | 1.00 | 73.43 | A |
| ATOM | 1249 | CB | VAL | A | 302 | −14.911 | 100.262 | −50.077 | 1.00 | 72.36 | A |
| ATOM | 1250 | CG1 | VAL | A | 302 | −14.996 | 100.251 | −51.586 | 1.00 | 70.53 | A |
| ATOM | 1251 | CG2 | VAL | A | 302 | −13.546 | 100.759 | −49.594 | 1.00 | 71.14 | A |
| ATOM | 1252 | C | VAL | A | 302 | −15.960 | 102.566 | −50.037 | 1.00 | 74.40 | A |
| ATOM | 1253 | O | VAL | A | 302 | −16.201 | 102.805 | −51.226 | 1.00 | 74.16 | A |
| ATOM | 1254 | N | ALA | A | 303 | −15.640 | 103.505 | −49.152 | 1.00 | 75.32 | A |
| ATOM | 1255 | CA | ALA | A | 303 | −15.534 | 104.914 | −49.509 | 1.00 | 76.08 | A |
| ATOM | 1256 | CB | ALA | A | 303 | −16.714 | 105.682 | −48.918 | 1.00 | 75.56 | A |
| ATOM | 1257 | C | ALA | A | 303 | −14.221 | 105.530 | −49.022 | 1.00 | 76.47 | A |
| ATOM | 1258 | O | ALA | A | 303 | −13.932 | 106.698 | −49.305 | 1.00 | 76.25 | A |
| ATOM | 1259 | N | GLY | A | 304 | −13.435 | 104.746 | −48.285 | 1.00 | 76.58 | A |
| ATOM | 1260 | CA | GLY | A | 304 | −12.169 | 105.235 | −47.767 | 1.00 | 76.19 | A |
| ATOM | 1261 | C | GLY | A | 304 | −11.184 | 105.537 | −48.877 | 1.00 | 76.64 | A |
| ATOM | 1262 | O | GLY | A | 304 | −10.771 | 106.687 | −49.059 | 1.00 | 76.62 | A |
| ATOM | 1263 | N | ALA | A | 305 | −10.812 | 104.495 | −49.620 | 1.00 | 76.61 | A |
| ATOM | 1264 | CA | ALA | A | 305 | −9.869 | 104.604 | −50.735 | 1.00 | 75.64 | A |
| ATOM | 1265 | CB | ALA | A | 305 | −8.622 | 105.396 | −50.308 | 1.00 | 75.16 | A |
| ATOM | 1266 | C | ALA | A | 305 | −9.459 | 103.219 | −51.240 | 1.00 | 75.04 | A |
| ATOM | 1267 | O | ALA | A | 305 | −8.422 | 102.690 | −50.847 | 1.00 | 74.28 | A |
| ATOM | 1268 | N | THR | A | 306 | −10.280 | 102.644 | −52.113 | 1.00 | 74.75 | A |
| ATOM | 1269 | CA | THR | A | 306 | −10.014 | 101.332 | −52.697 | 1.00 | 73.89 | A |
| ATOM | 1270 | CB | THR | A | 306 | −11.309 | 100.478 | −52.749 | 1.00 | 73.35 | A |
| ATOM | 1271 | OG1 | THR | A | 306 | −11.048 | 99.229 | −53.402 | 1.00 | 73.02 | A |
| ATOM | 1272 | OG2 | THR | A | 306 | −12.404 | 101.218 | −53.494 | 1.00 | 72.10 | A |
| ATOM | 1273 | C | THR | A | 306 | −9.455 | 101.520 | −54.114 | 1.00 | 74.01 | A |
| ATOM | 1274 | O | THR | A | 306 | −9.854 | 102.445 | −54.828 | 1.00 | 73.10 | A |
| ATOM | 1275 | N | MET | A | 307 | −8.530 | 100.648 | −54.513 | 1.00 | 74.07 | A |
| ATOM | 1276 | CA | MET | A | 307 | −7.901 | 100.726 | −55.831 | 1.00 | 74.12 | A |
| ATOM | 1277 | CB | MET | A | 307 | −6.632 | 101.565 | −55.712 | 1.00 | 73.12 | A |
| ATOM | 1278 | CG | MET | A | 307 | −5.784 | 101.217 | −54.489 | 1.00 | 70.93 | A |
| ATOM | 1279 | SD | MET | A | 307 | −4.511 | 102.458 | −54.104 | 1.00 | 68.51 | A |
| ATOM | 1280 | CE | MET | A | 307 | −4.504 | 102.352 | −52.251 | 1.00 | 66.70 | A |
| ATOM | 1281 | C | MET | A | 307 | −7.585 | 99.334 | −56.409 | 1.00 | 75.82 | A |
| ATOM | 1282 | O | MET | A | 307 | −6.649 | 99.165 | −57.209 | 1.00 | 74.61 | A |
| ATOM | 1283 | N | THR | A | 309 | −8.399 | 98.360 | −55.993 | 1.00 | 77.31 | A |
| ATOM | 1284 | CA | THR | A | 309 | −8.314 | 96.947 | −56.383 | 1.00 | 78.49 | A |
| ATOM | 1285 | CB | THR | A | 309 | −9.661 | 96.244 | −56.056 | 1.00 | 78.84 | A |
| ATOM | 1286 | OG1 | THR | A | 309 | −10.008 | 96.517 | −54.694 | 1.00 | 79.62 | A |
| ATOM | 1287 | CG2 | THR | A | 309 | −9.572 | 94.733 | −56.246 | 1.00 | 78.86 | A |
| ATOM | 1288 | C | THR | A | 309 | −7.959 | 96.679 | −57.852 | 1.00 | 78.75 | A |
| ATOM | 1289 | O | THR | A | 309 | −7.735 | 97.604 | −58.634 | 1.00 | 78.84 | A |
| ATOM | 1290 | N | PHE | A | 310 | −7.898 | 95.400 | −58.213 | 1.00 | 79.13 | A |
| ATOM | 1291 | CA | PHE | A | 310 | −7.588 | 94.997 | −59.579 | 1.00 | 79.15 | A |
| ATOM | 1292 | CB | PHE | A | 310 | −6.231 | 94.252 | −59.643 | 1.00 | 77.42 | A |
| ATOM | 1293 | CG | PHE | A | 310 | −5.022 | 95.139 | −59.420 | 1.00 | 75.20 | A |
| ATOM | 1294 | CD1 | PHE | A | 310 | −4.671 | 95.569 | −58.140 | 1.00 | 74.27 | A |
| ATOM | 1295 | CD2 | PHE | A | 310 | −4.255 | 95.577 | −60.500 | 1.00 | 73.64 | A |
| ATOM | 1296 | CE1 | PHE | A | 310 | −3.563 | 96.428 | −57.938 | 1.00 | 71.91 | A |
| ATOM | 1297 | CE2 | PHE | A | 310 | −3.151 | 96.434 | −60.311 | 1.00 | 71.10 | A |
| ATOM | 1298 | CZ | PHE | A | 310 | −2.810 | 96.862 | −59.026 | 1.00 | 69.93 | A |
| ATOM | 1299 | C | PHE | A | 310 | −8.696 | 94.109 | −60.174 | 1.00 | 80.19 | A |
| ATOM | 1300 | O | PHE | A | 310 | −8.958 | 93.002 | −59.690 | 1.00 | 80.39 | A |
| ATOM | 1301 | N | CYS | A | 311 | −9.343 | 94.635 | −61.217 | 1.00 | 80.36 | A |
| ATOM | 1302 | CA | CYS | A | 311 | −10.408 | 93.978 | −61.994 | 1.00 | 79.60 | A |
| ATOM | 1303 | CB | CYS | A | 311 | −11.609 | 93.541 | −61.131 | 1.00 | 80.92 | A |
| ATOM | 1304 | SG | CYS | A | 311 | −12.941 | 92.618 | −62.081 | 1.00 | 81.68 | A |
| ATOM | 1305 | C | CYS | A | 311 | −10.862 | 95.035 | −62.996 | 1.00 | 78.12 | A |
| ATOM | 1306 | O | CYS | A | 311 | −11.009 | 96.210 | −62.637 | 1.00 | 77.53 | A |
| ATOM | 1307 | N | GLY | A | 312 | −11.084 | 94.633 | −64.247 | 1.00 | 76.21 | A |
| ATOM | 1308 | CA | GLY | A | 312 | −11.486 | 95.616 | −65.232 | 1.00 | 72.84 | A |
| ATOM | 1309 | C | GLY | A | 312 | −12.188 | 95.222 | −66.517 | 1.00 | 70.38 | A |
| ATOM | 1310 | O | GLY | A | 312 | −12.515 | 94.070 | −66.778 | 1.00 | 69.42 | A |
| ATOM | 1311 | N | THR | A | 313 | −12.420 | 96.248 | −67.321 | 1.00 | 68.96 | A |
| ATOM | 1312 | CA | THR | A | 313 | −13.082 | 96.131 | −68.607 | 1.00 | 68.24 | A |
| ATOM | 1313 | CB | THR | A | 313 | −14.458 | 96.839 | −68.579 | 1.00 | 67.34 | A |
| ATOM | 1314 | OG1 | THR | A | 313 | −15.344 | 96.121 | −67.712 | 1.00 | 65.56 | A |
| ATOM | 1315 | CG2 | THR | A | 313 | −15.053 | 96.919 | −69.981 | 1.00 | 67.08 | A |
| ATOM | 1316 | C | THR | A | 313 | −12.198 | 96.781 | −69.680 | 1.00 | 67.17 | A |
| ATOM | 1317 | O | THR | A | 313 | −12.183 | 98.004 | −69.839 | 1.00 | 66.98 | A |
| ATOM | 1318 | N | PRO | A | 314 | −11.468 | 95.958 | −70.438 | 1.00 | 65.72 | A |
| ATOM | 1319 | CD | PRO | A | 314 | −11.843 | 94.553 | −70.688 | 1.00 | 67.12 | A |
| ATOM | 1320 | CA | PRO | A | 314 | −10.583 | 96.436 | −71.491 | 1.00 | 65.27 | A |
| ATOM | 1321 | CB | PRO | A | 314 | −11.033 | 95.607 | −72.677 | 1.00 | 65.64 | A |
| ATOM | 1322 | CG | PRO | A | 314 | −11.198 | 94.260 | −72.048 | 1.00 | 67.64 | A |
| ATOM | 1323 | C | PRO | A | 314 | −10.618 | 97.937 | −71.764 | 1.00 | 63.03 | A |
| ATOM | 1324 | O | PRO | A | 314 | −10.238 | 98.754 | −70.921 | 1.00 | 62.60 | A |
| ATOM | 1325 | N | GLU | A | 315 | −11.076 | 98.280 | −72.960 | 1.00 | 61.49 | A |
| ATOM | 1326 | CA | GLU | A | 315 | −11.192 | 99.665 | −73.436 | 1.00 | 58.28 | A |
| ATOM | 1327 | CB | GLU | A | 315 | −11.868 | 99.660 | −74.815 | 1.00 | 56.91 | A |
| ATOM | 1328 | CG | GLU | A | 315 | −11.520 | 98.451 | −75.708 | 1.00 | 56.55 | A |
| ATOM | 1329 | CD | GLU | A | 315 | −11.871 | 97.082 | −75.090 | 1.00 | 55.97 | A |
| ATOM | 1330 | OE1 | GLU | A | 315 | −12.845 | 96.976 | −74.315 | 1.00 | 54.66 | A |
| ATOM | 1331 | OE2 | GLU | A | 315 | −11.177 | 96.096 | −75.401 | 1.00 | 54.71 | A |
| ATOM | 1332 | C | GLU | A | 315 | −11.975 | 100.605 | −72.493 | 1.00 | 55.25 | A |
| ATOM | 1333 | O | GLU | A | 315 | −12.296 | 101.726 | −72.871 | 1.00 | 53.46 | A |
| ATOM | 1334 | N | TYR | A | 316 | −12.258 | 100.147 | −71.274 | 1.00 | 53.16 | A |
| ATOM | 1335 | CA | TYR | A | 316 | −13.021 | 100.924 | −70.298 | 1.00 | 51.85 | A |
| ATOM | 1336 | CB | TYR | A | 316 | −14.293 | 100.167 | −69.887 | 1.00 | 54.53 | A |
| ATOM | 1337 | CG | TYR | A | 316 | −15.527 | 100.441 | −70.719 | 1.00 | 56.84 | A |
| ATOM | 1338 | CD1 | TYR | A | 316 | −15.838 | 99.647 | −71.833 | 1.00 | 56.81 | A |
| ATOM | 1339 | CE1 | TYR | A | 316 | −16.980 | 99.894 | −72.596 | 1.00 | 56.47 | A |
| ATOM | 1340 | CD2 | TYR | A | 316 | −16.395 | 101.492 | −70.389 | 1.00 | 56.21 | A |
| ATOM | 1341 | CE2 | TYR | A | 316 | −17.539 | 101.744 | −71.151 | 1.00 | 55.90 | A |
| ATOM | 1342 | CZ | TYR | A | 316 | −17.821 | 100.943 | −72.253 | 1.00 | 55.67 | A |
| ATOM | 1343 | OH | TYR | A | 316 | −18.921 | 101.204 | −73.037 | 1.00 | 56.04 | A |
| ATOM | 1344 | C | TYR | A | 316 | −12.309 | 101.329 | −69.003 | 1.00 | 47.86 | A |
| ATOM | 1345 | O | TYR | A | 316 | −12.642 | 102.357 | −68.421 | 1.00 | 48.81 | A |
| ATOM | 1346 | N | LEU | A | 317 | −11.370 | 100.525 | −68.528 | 1.00 | 41.96 | A |
| ATOM | 1347 | CA | LEU | A | 317 | −10.702 | 100.868 | −67.283 | 1.00 | 38.08 | A |
| ATOM | 1348 | CB | LEU | A | 317 | −9.550 | 99.891 | −67.001 | 1.00 | 36.93 | A |
| ATOM | 1349 | CG | LEU | A | 317 | −9.941 | 98.607 | −66.256 | 1.00 | 34.10 | A |
| ATOM | 1350 | CD1 | LEU | A | 317 | −8.820 | 98.231 | −65.299 | 1.00 | 33.28 | A |
| ATOM | 1351 | CD2 | LEU | A | 317 | −11.216 | 98.815 | −65.471 | 1.00 | 32.46 | A |
| ATOM | 1352 | C | LEU | A | 317 | −10.212 | 102.319 | −67.224 | 1.00 | 35.05 | A |
| ATOM | 1353 | O | LEU | A | 317 | −9.776 | 102.871 | −68.223 | 1.00 | 33.58 | A |
| ATOM | 1354 | N | ALA | A | 318 | −10.304 | 102.927 | −66.044 | 1.00 | 32.34 | A |
| ATOM | 1355 | CA | ALA | A | 318 | −9.905 | 104.310 | −65.854 | 1.00 | 34.07 | A |
| ATOM | 1356 | CB | ALA | A | 318 | −10.747 | 104.933 | −64.748 | 1.00 | 32.53 | A |
| ATOM | 1357 | C | ALA | A | 318 | −8.410 | 104.471 | −65.543 | 1.00 | 35.01 | A |
| ATOM | 1358 | O | ALA | A | 318 | −7.841 | 103.700 | −64.773 | 1.00 | 37.39 | A |
| ATOM | 1359 | N | PRO | A | 319 | −7.755 | 105.487 | −66.129 | 1.00 | 33.94 | A |
| ATOM | 1360 | CD | PRO | A | 319 | −8.257 | 106.556 | −67.005 | 1.00 | 34.01 | A |
| ATOM | 1361 | CA | PRO | A | 319 | −6.329 | 105.665 | −65.857 | 1.00 | 33.57 | A |
| ATOM | 1362 | CB | PRO | A | 319 | −6.020 | 107.053 | −66.434 | 1.00 | 33.33 | A |
| ATOM | 1363 | CG | PRO | A | 319 | −7.367 | 107.700 | −66.611 | 1.00 | 34.75 | A |
| ATOM | 1364 | C | PRO | A | 319 | −5.924 | 105.520 | −64.404 | 1.00 | 34.03 | A |
| ATOM | 1365 | O | PRO | A | 319 | −5.033 | 104.739 | −64.095 | 1.00 | 34.93 | A |
| ATOM | 1366 | N | GLU | A | 320 | −6.571 | 106.254 | −63.506 | 1.00 | 35.74 | A |
| ATOM | 1367 | CA | GLU | A | 320 | −6.216 | 106.168 | −62.089 | 1.00 | 36.67 | A |
| ATOM | 1368 | CB | GLU | A | 320 | −7.027 | 107.166 | −61.243 | 1.00 | 36.20 | A |
| ATOM | 1369 | CG | GLU | A | 320 | −8.520 | 106.843 | −61.147 | 1.00 | 36.13 | A |
| ATOM | 1370 | CD | GLU | A | 320 | −9.344 | 107.541 | −62.200 | 1.00 | 36.06 | A |
| ATOM | 1371 | OE1 | GLU | A | 320 | −8.959 | 107.535 | −63.383 | 1.00 | 36.56 | A |
| ATOM | 1372 | OE2 | GLU | A | 320 | −10.390 | 108.096 | −61.839 | 1.00 | 35.80 | A |
| ATOM | 1373 | C | GLU | A | 320 | −6.443 | 104.740 | −61.576 | 1.00 | 37.87 | A |
| ATOM | 1374 | O | GLU | A | 320 | −5.766 | 104.289 | −60.646 | 1.00 | 38.06 | A |
| ATOM | 1375 | N | VAL | A | 321 | −7.399 | 104.030 | −62.169 | 1.00 | 36.31 | A |
| ATOM | 1376 | CA | VAL | A | 321 | −7.638 | 102.667 | −61.755 | 1.00 | 36.60 | A |
| ATOM | 1377 | CB | VAL | A | 321 | −8.908 | 102.086 | −62.396 | 1.00 | 36.12 | A |
| ATOM | 1378 | CG1 | VAL | A | 321 | −8.998 | 100.595 | −62.123 | 1.00 | 33.41 | A |
| ATOM | 1379 | CG2 | VAL | A | 321 | −10.136 | 102.787 | −61.827 | 1.00 | 35.14 | A |
| ATOM | 1380 | C | VAL | A | 321 | −6.431 | 101.884 | −62.229 | 1.00 | 38.65 | A |
| ATOM | 1381 | O | VAL | A | 321 | −6.037 | 100.902 | −61.605 | 1.00 | 41.18 | A |
| ATOM | 1382 | N | LEU | A | 322 | −5.821 | 102.335 | −63.322 | 1.00 | 39.36 | A |
| ATOM | 1383 | CA | LEU | A | 322 | −4.654 | 101.649 | −63.880 | 1.00 | 39.61 | A |
| ATOM | 1384 | CB | LEU | A | 322 | −4.520 | 101.940 | −65.381 | 1.00 | 37.36 | A |
| ATOM | 1385 | CG | LEU | A | 322 | −5.685 | 101.511 | −66.276 | 1.00 | 35.40 | A |
| ATOM | 1386 | CD1 | LEU | A | 322 | −5.399 | 101.809 | −67.755 | 1.00 | 32.58 | A |
| ATOM | 1387 | CD2 | LEU | A | 322 | −5.922 | 100.042 | −66.051 | 1.00 | 33.55 | A |
| ATOM | 1388 | C | LEU | A | 322 | −3.348 | 102.000 | −63.186 | 1.00 | 40.65 | A |
| ATOM | 1389 | O | LEU | A | 322 | −2.602 | 101.124 | −62.768 | 1.00 | 40.96 | A |
| ATOM | 1390 | N | GLU | A | 323 | −3.065 | 103.287 | −63.071 | 1.00 | 42.76 | A |
| ATOM | 1391 | CA | GLU | A | 323 | −1.831 | 103.736 | −62.434 | 1.00 | 44.83 | A |
| ATOM | 1392 | CB | GLU | A | 323 | −1.688 | 105.257 | −62.624 | 1.00 | 44.50 | A |
| ATOM | 1393 | CG | GLU | A | 323 | −0.305 | 105.846 | −62.381 | 1.00 | 45.98 | A |
| ATOM | 1394 | CD | GLU | A | 323 | −0.173 | 107.299 | −62.892 | 1.00 | 47.67 | A |
| ATOM | 1395 | OE1 | GLU | A | 323 | −1.042 | 108.160 | −62.586 | 1.00 | 47.60 | A |
| ATOM | 1396 | OE2 | GLU | A | 323 | 0.818 | 107.592 | −63.595 | 1.00 | 46.81 | A |
| ATOM | 1397 | C | GLU | A | 323 | −1.906 | 103.364 | −60.958 | 1.00 | 46.00 | A |
| ATOM | 1398 | O | GLU | A | 323 | −1.192 | 103.917 | −60.140 | 1.00 | 46.72 | A |
| ATOM | 1399 | N | ASP | A | 324 | −2.784 | 102.417 | −60.634 | 1.00 | 47.83 | A |
| ATOM | 1400 | CA | ASP | A | 324 | −2.994 | 101.950 | −59.261 | 1.00 | 49.37 | A |
| ATOM | 1401 | CB | ASP | A | 324 | −1.683 | 101.529 | −58.600 | 1.00 | 51.34 | A |
| ATOM | 1402 | CG | ASP | A | 324 | −0.872 | 100.577 | −59.450 | 1.00 | 51.23 | A |
| ATOM | 1403 | OD1 | ASP | A | 324 | −1.174 | 99.348 | −59.467 | 1.00 | 49.81 | A |
| ATOM | 1404 | OD2 | ASP | A | 324 | 0.073 | 101.078 | −60.093 | 1.00 | 50.14 | A |
| ATOM | 1405 | C | ASP | A | 324 | −3.590 | 103.048 | −58.426 | 1.00 | 49.80 | A |
| ATOM | 1406 | O | ASP | A | 324 | −4.109 | 102.787 | −57.346 | 1.00 | 48.34 | A |
| ATOM | 1407 | N | ASN | A | 325 | −3.480 | 104.276 | −58.923 | 1.00 | 52.04 | A |
| ATOM | 1408 | CA | ASN | A | 325 | −4.002 | 105.450 | −58.234 | 1.00 | 56.14 | A |
| ATOM | 1409 | CB | ASN | A | 325 | −4.011 | 106.655 | −59.169 | 1.00 | 58.77 | A |
| ATOM | 1410 | CG | ASN | A | 325 | −2.976 | 107.692 | −58.789 | 1.00 | 60.00 | A |
| ATOM | 1411 | OD1 | ASN | A | 325 | −1.765 | 107.401 | −58.726 | 1.00 | 59.63 | A |
| ATOM | 1412 | ND2 | ASN | A | 325 | −3.446 | 108.917 | −58.525 | 1.00 | 59.01 | A |
| ATOM | 1413 | C | ASN | A | 325 | −5.404 | 105.200 | −57.713 | 1.00 | 57.52 | A |
| ATOM | 1414 | O | ASN | A | 325 | −6.342 | 105.001 | −58.480 | 1.00 | 56.92 | A |
| ATOM | 1415 | N | ASP | A | 326 | −5.532 | 105.214 | −56.392 | 1.00 | 58.30 | A |
| ATOM | 1416 | CA | ASP | A | 326 | −6.806 | 104.958 | −55.745 | 1.00 | 57.19 | A |
| ATOM | 1417 | CB | ASP | A | 326 | −6.778 | 105.439 | −54.293 | 1.00 | 57.07 | A |
| ATOM | 1418 | CG | ASP | A | 326 | −7.366 | 104.422 | −53.343 | 1.00 | 56.28 | A |
| ATOM | 1419 | OD1 | ASP | A | 326 | −8.436 | 103.856 | −53.675 | 1.00 | 54.26 | A |
| ATOM | 1420 | OD2 | ASP | A | 326 | −6.772 | 104.197 | −52.269 | 1.00 | 56.03 | A |
| ATOM | 1421 | C | ASP | A | 326 | −7.923 | 105.640 | −56.496 | 1.00 | 55.86 | A |
| ATOM | 1422 | O | ASP | A | 326 | −7.730 | 106.702 | −57.079 | 1.00 | 56.11 | A |
| ATOM | 1423 | N | TYR | A | 327 | −9.092 | 105.018 | −56.481 | 1.00 | 55.34 | A |
| ATOM | 1424 | CA | TYR | A | 327 | −10.236 | 105.574 | −57.178 | 1.00 | 54.96 | A |
| ATOM | 1425 | CB | TYR | A | 327 | −10.660 | 104.630 | −58.300 | 1.00 | 55.94 | A |
| ATOM | 1426 | CG | TYR | A | 327 | −11.246 | 103.330 | −57.820 | 1.00 | 55.51 | A |
| ATOM | 1427 | CD1 | TYR | A | 327 | −12.449 | 103.299 | −57.126 | 1.00 | 54.72 | A |
| ATOM | 1428 | CE1 | TYR | A | 327 | −12.989 | 102.114 | −56.698 | 1.00 | 56.28 | A |
| ATOM | 1429 | CD2 | TYR | A | 327 | −10.601 | 102.131 | −58.071 | 1.00 | 56.68 | A |
| ATOM | 1430 | CE2 | TYR | A | 327 | −11.134 | 100.933 | −57.646 | 1.00 | 57.44 | A |
| ATOM | 1431 | CZ | TYR | A | 327 | −12.326 | 100.930 | −56.958 | 1.00 | 57.63 | A |
| ATOM | 1432 | OH | TYR | A | 327 | −12.835 | 99.734 | −56.501 | 1.00 | 59.73 | A |
| ATOM | 1433 | C | TYR | A | 327 | −11.439 | 105.889 | −56.284 | 1.00 | 53.95 | A |
| ATOM | 1434 | O | TYR | A | 327 | −11.480 | 105.544 | −55.100 | 1.00 | 55.45 | A |
| ATOM | 1435 | N | GLY | A | 328 | −12.422 | 106.547 | −56.875 | 1.00 | 50.71 | A |
| ATOM | 1436 | CA | GLY | A | 328 | −13.612 | 106.912 | −56.141 | 1.00 | 48.37 | A |
| ATOM | 1437 | C | GLY | A | 328 | −14.716 | 107.059 | −57.150 | 1.00 | 46.26 | A |
| ATOM | 1438 | O | GLY | A | 328 | −14.754 | 106.292 | −58.111 | 1.00 | 46.78 | A |
| ATOM | 1439 | N | ARG | A | 329 | −15.591 | 108.044 | −56.966 | 1.00 | 41.48 | A |
| ATOM | 1440 | CA | ARG | A | 329 | −16.682 | 108.219 | −57.903 | 1.00 | 37.24 | A |
| ATOM | 1441 | CB | ARG | A | 329 | −17.587 | 109.383 | −57.513 | 1.00 | 37.63 | A |
| ATOM | 1442 | CG | ARG | A | 329 | −17.764 | 109.646 | −56.057 | 1.00 | 36.06 | A |
| ATOM | 1443 | CD | ARG | A | 329 | −16.844 | 110.758 | −55.629 | 1.00 | 35.26 | A |
| ATOM | 1444 | NE | ARG | A | 329 | −16.817 | 111.840 | −56.601 | 1.00 | 33.75 | A |
| ATOM | 1445 | CZ | ARG | A | 329 | −16.106 | 112.955 | −56.456 | 1.00 | 35.19 | A |
| ATOM | 1446 | NH1 | ARG | A | 329 | −15.364 | 113.145 | −55.374 | 1.00 | 34.20 | A |
| ATOM | 1447 | NH2 | ARG | A | 329 | −16.121 | 113.882 | −57.400 | 1.00 | 36.43 | A |
| ATOM | 1448 | C | ARG | A | 329 | −16.149 | 108.487 | −59.298 | 1.00 | 35.05 | A |
| ATOM | 1449 | O | ARG | A | 329 | −16.646 | 107.927 | −60.263 | 1.00 | 36.56 | A |
| ATOM | 1450 | N | ALA | A | 330 | −15.136 | 109.340 | −59.393 | 1.00 | 31.90 | A |
| ATOM | 1451 | CA | ALA | A | 330 | −14.550 | 109.723 | −60.669 | 1.00 | 30.63 | A |
| ATOM | 1452 | CB | ALA | A | 330 | −13.257 | 110.459 | −60.441 | 1.00 | 30.72 | A |
| ATOM | 1453 | C | ALA | A | 330 | −14.336 | 108.644 | −61.715 | 1.00 | 29.98 | A |
| ATOM | 1454 | O | ALA | A | 330 | −14.145 | 108.974 | −62.867 | 1.00 | 31.64 | A |
| ATOM | 1455 | N | VAL | A | 331 | −14.342 | 107.366 | −61.352 | 1.00 | 29.53 | A |
| ATOM | 1456 | CA | VAL | A | 331 | −14.169 | 106.318 | −62.367 | 1.00 | 27.58 | A |
| ATOM | 1457 | CB | VAL | A | 331 | −13.489 | 105.011 | −61.808 | 1.00 | 26.39 | A |
| ATOM | 1458 | CG1 | VAL | A | 331 | −12.252 | 105.376 | −61.037 | 1.00 | 26.09 | A |
| ATOM | 1459 | CG2 | VAL | A | 331 | −14.454 | 104.199 | −60.955 | 1.00 | 23.49 | A |
| ATOM | 1460 | C | VAL | A | 331 | −15.529 | 105.954 | −62.962 | 1.00 | 26.82 | A |
| ATOM | 1461 | O | VAL | A | 331 | −15.608 | 105.294 | −63.999 | 1.00 | 28.10 | A |
| ATOM | 1462 | N | ASP | A | 332 | −16.596 | 106.380 | −62.296 | 1.00 | 23.56 | A |
| ATOM | 1463 | CA | ASP | A | 332 | −17.939 | 106.120 | −62.777 | 1.00 | 24.79 | A |
| ATOM | 1464 | CB | ASP | A | 332 | −18.950 | 106.312 | −61.647 | 1.00 | 25.46 | A |
| ATOM | 1465 | CG | ASP | A | 332 | −19.033 | 105.123 | −60.729 | 1.00 | 27.62 | A |
| ATOM | 1466 | OD1 | ASP | A | 332 | −19.285 | 104.018 | −61.215 | 1.00 | 30.62 | A |
| ATOM | 1467 | OD2 | ASP | A | 332 | −18.854 | 105.276 | −59.513 | 1.00 | 29.68 | A |
| ATOM | 1468 | C | ASP | A | 332 | −18.221 | 107.115 | −63.897 | 1.00 | 25.01 | A |
| ATOM | 1469 | O | ASP | A | 332 | −18.965 | 106.854 | −64.833 | 1.00 | 26.25 | A |
| ATOM | 1470 | N | TRP | A | 333 | −17.594 | 108.266 | −63.777 | 1.00 | 24.60 | A |
| ATOM | 1471 | CA | TRP | A | 333 | −17.748 | 109.336 | −64.722 | 1.00 | 23.25 | A |
| ATOM | 1472 | CB | TRP | A | 333 | −17.236 | 110.606 | −64.076 | 1.00 | 25.29 | A |
| ATOM | 1473 | CG | TRP | A | 333 | −18.122 | 111.016 | −62.956 | 1.00 | 26.30 | A |
| ATOM | 1474 | CD2 | TRP | A | 333 | −19.558 | 111.047 | −62.975 | 1.00 | 26.84 | A |
| ATOM | 1475 | CE2 | TRP | A | 333 | −19.979 | 111.535 | −61.730 | 1.00 | 27.00 | A |
| ATOM | 1476 | CE3 | TRP | A | 333 | −20.530 | 110.712 | −63.934 | 1.00 | 27.23 | A |
| ATOM | 1477 | CD1 | TRP | A | 333 | −17.745 | 111.465 | −61.736 | 1.00 | 27.59 | A |
| ATOM | 1478 | NE1 | TRP | A | 333 | −18.854 | 111.784 | −60.988 | 1.00 | 29.34 | A |
| ATOM | 1479 | CZ2 | TRP | A | 333 | −21.330 | 111.698 | −61.412 | 1.00 | 25.21 | A |
| ATOM | 1480 | CZ3 | TRP | A | 333 | −21.867 | 110.875 | −63.616 | 1.00 | 24.16 | A |
| ATOM | 1481 | CH2 | TRP | A | 333 | −22.254 | 111.366 | −62.365 | 1.00 | 23.45 | A |
| ATOM | 1482 | C | TRP | A | 333 | −17.004 | 109.000 | −65.965 | 1.00 | 23.53 | A |
| ATOM | 1483 | O | TRP | A | 333 | −17.494 | 109.228 | −67.063 | 1.00 | 23.81 | A |
| ATOM | 1484 | N | TRP | A | 334 | −15.807 | 108.452 | −65.772 | 1.00 | 24.61 | A |
| ATOM | 1485 | CA | TRP | A | 334 | −14.938 | 107.993 | −66.857 | 1.00 | 22.69 | A |
| ATOM | 1486 | CB | TRP | A | 334 | −13.617 | 107.483 | −66.258 | 1.00 | 23.94 | A |
| ATOM | 1487 | CG | TRP | A | 334 | −12.796 | 106.588 | −67.171 | 1.00 | 27.74 | A |
| ATOM | 1488 | CD2 | TRP | A | 334 | −11.743 | 106.998 | −68.049 | 1.00 | 28.58 | A |
| ATOM | 1489 | CE2 | TRP | A | 334 | −11.309 | 105.852 | −68.742 | 1.00 | 27.50 | A |
| ATOM | 1490 | CE3 | TRP | A | 334 | −11.125 | 108.229 | −68.321 | 1.00 | 29.45 | A |
| ATOM | 1491 | CD1 | TRP | A | 334 | −12.945 | 105.233 | −67.366 | 1.00 | 24.94 | A |
| ATOM | 1492 | NE1 | TRP | A | 334 | −12.059 | 104.793 | −68.302 | 1.00 | 25.22 | A |
| ATOM | 1493 | CZ2 | TEP | A | 334 | −10.288 | 105.900 | −69.686 | 1.00 | 28.75 | A |
| ATOM | 1494 | CZ3 | TRP | A | 334 | −10.109 | 108.274 | −69.259 | 1.00 | 28.55 | A |
| ATOM | 1495 | CH2 | TRP | A | 334 | −9.703 | 107.120 | −69.930 | 1.00 | 29.40 | A |
| ATOM | 1496 | C | TRP | A | 334 | −15.682 | 106.872 | −67.607 | 1.00 | 20.19 | A |
| ATOM | 1497 | O | TRP | A | 334 | −15.746 | 106.845 | −68.824 | 1.00 | 17.98 | A |
| ATOM | 1498 | N | GLY | A | 335 | −16.265 | 105.956 | −66.855 | 1.00 | 18.67 | A |
| ATOM | 1499 | CA | GLY | A | 335 | −17.004 | 104.878 | −67.467 | 1.00 | 20.33 | A |
| ATOM | 1500 | C | GLY | A | 335 | −18.069 | 105.391 | −68.400 | 1.00 | 21.63 | A |
| ATOM | 1501 | O | GLY | A | 335 | −18.236 | 104.862 | −69.489 | 1.00 | 23.51 | A |
| ATOM | 1502 | N | LEU | A | 336 | −18.787 | 106.421 | −67.963 | 1.00 | 23.99 | A |
| ATOM | 1503 | CA | LEU | A | 336 | −19.863 | 107.049 | −68.726 | 1.00 | 24.84 | A |
| ATOM | 1504 | CB | LEU | A | 336 | −20.535 | 108.121 | −67.873 | 1.00 | 24.67 | A |
| ATOM | 1505 | CG | LEU | A | 336 | −21.839 | 108.729 | −68.394 | 1.00 | 25.55 | A |
| ATOM | 1506 | CD1 | LEU | A | 336 | −22.988 | 107.739 | −68.170 | 1.00 | 25.24 | A |
| ATOM | 1507 | CD2 | LEU | A | 336 | −22.103 | 110.052 | −67.685 | 1.00 | 23.57 | A |
| ATOM | 1508 | C | LEU | A | 336 | −19.363 | 107.687 | −70.018 | 1.00 | 26.87 | A |
| ATOM | 1509 | O | LEU | A | 336 | −19.949 | 107.499 | −71.078 | 1.00 | 27.17 | A |
| ATOM | 1510 | N | GLY | A | 337 | −18.288 | 108.458 | −69.911 | 1.00 | 27.61 | A |
| ATOM | 1511 | CA | GLY | A | 337 | −17.717 | 109.106 | −71.073 | 1.00 | 29.23 | A |
| ATOM | 1512 | C | GLY | A | 337 | −17.258 | 108.141 | −72.149 | 1.00 | 29.61 | A |
| ATOM | 1513 | O | GLY | A | 337 | −17.309 | 108.459 | −73.331 | 1.00 | 30.14 | A |
| ATOM | 1514 | N | VAL | A | 338 | −16.790 | 106.966 | −71.737 | 1.00 | 30.37 | A |
| ATOM | 1515 | CA | VAL | A | 338 | −16.342 | 105.942 | −72.679 | 1.00 | 30.86 | A |
| ATOM | 1516 | CB | VAL | A | 338 | −15.590 | 104.784 | −71.960 | 1.00 | 31.23 | A |
| ATOM | 1517 | CG1 | VAL | A | 338 | −15.493 | 103.583 | −72.862 | 1.00 | 29.09 | A |
| ATOM | 1518 | CG2 | VAL | A | 338 | −14.181 | 105.224 | −71.591 | 1.00 | 30.70 | A |
| ATOM | 1519 | C | VAL | A | 338 | −17.579 | 105.403 | −73.372 | 1.00 | 30.82 | A |
| ATOM | 1520 | O | VAL | A | 338 | −17.595 | 105.235 | −74.584 | 1.00 | 31.75 | A |
| ATOM | 1521 | N | VAL | A | 339 | −18.625 | 105.152 | −72.596 | 1.00 | 31.86 | A |
| ATOM | 1522 | CA | VAL | A | 339 | −19.879 | 104.661 | −73.149 | 1.00 | 34.51 | A |
| ATOM | 1523 | CB | VAL | A | 339 | −20.935 | 104.438 | −72.036 | 1.00 | 34.77 | A |
| ATOM | 1524 | CG1 | VAL | A | 339 | −22.165 | 103.763 | −72.607 | 1.00 | 33.66 | A |
| ATOM | 1525 | CG2 | VAL | A | 339 | −20.354 | 103.587 | −70.938 | 1.00 | 34.82 | A |
| ATOM | 1526 | C | VAL | A | 339 | −20.420 | 105.697 | −74.146 | 1.00 | 36.29 | A |
| ATOM | 1527 | O | VAL | A | 339 | −20.900 | 105.353 | −75.217 | 1.00 | 36.95 | A |
| ATOM | 1528 | N | MET | A | 340 | −20.329 | 106.972 | −73.792 | 1.00 | 37.21 | A |
| ATOM | 1529 | CA | MET | A | 340 | −20.818 | 108.025 | −74.668 | 1.00 | 38.32 | A |
| ATOM | 1530 | CB | MET | A | 340 | −20.970 | 109.322 | −73.907 | 1.00 | 37.63 | A |
| ATOM | 1531 | CG | MET | A | 340 | −22.325 | 109.906 | −74.068 | 1.00 | 38.53 | A |
| ATOM | 1532 | SD | MET | A | 340 | −23.123 | 109.959 | −72.510 | 1.00 | 39.47 | A |
| ATOM | 1533 | CE | MET | A | 340 | −21.792 | 110.826 | −71.582 | 1.00 | 38.30 | A |
| ATOM | 1534 | C | MET | A | 340 | −19.911 | 108.265 | −75.855 | 1.00 | 38.66 | A |
| ATOM | 1535 | O | MET | A | 340 | −20.297 | 108.919 | −76.820 | 1.00 | 39.35 | A |
| ATOM | 1536 | N | TYR | A | 341 | −18.690 | 107.761 | −75.780 | 1.00 | 37.91 | A |
| ATOM | 1537 | CA | TYR | A | 341 | −17.792 | 107.937 | −76.902 | 1.00 | 37.35 | A |
| ATOM | 1538 | CB | TYR | A | 341 | −16.362 | 107.567 | −76.548 | 1.00 | 35.13 | A |
| ATOM | 1539 | CG | TYR | A | 341 | −15.423 | 107.906 | −77.662 | 1.00 | 34.49 | A |
| ATOM | 1540 | CD1 | TYR | A | 341 | −15.518 | 107.270 | −78.899 | 1.00 | 33.81 | A |
| ATOM | 1541 | CE1 | TYR | A | 341 | −14.680 | 107.618 | −79.950 | 1.00 | 35.18 | A |
| ATOM | 1542 | CD2 | TYR | A | 341 | −14.464 | 108.900 | −77.501 | 1.00 | 35.70 | A |
| ATOM | 1543 | CE2 | TYR | A | 341 | −13.615 | 109.261 | −78.547 | 1.00 | 36.06 | A |
| ATOM | 1544 | CZ | TYR | A | 341 | −13.726 | 108.618 | −79.768 | 1.00 | 35.95 | A |
| ATOM | 1545 | OH | TYR | A | 341 | −12.888 | 108.981 | −80.797 | 1.00 | 36.52 | A |
| ATOM | 1546 | C | TYR | A | 341 | −18.285 | 106.987 | −77.959 | 1.00 | 37.38 | A |
| ATOM | 1547 | O | TYR | A | 341 | −18.443 | 107.347 | −79.118 | 1.00 | 38.53 | A |
| ATOM | 1548 | N | GLU | A | 342 | −18.531 | 105.760 | −77.532 | 1.00 | 35.93 | A |
| ATOM | 1549 | CA | GLU | A | 342 | −19.002 | 104.728 | −78.416 | 1.00 | 35.30 | A |
| ATOM | 1550 | CB | GLU | A | 342 | −18.995 | 103.402 | −77.648 | 1.00 | 36.37 | A |
| ATOM | 1551 | CG | GLU | A | 342 | −19.528 | 102.192 | −78.390 | 1.00 | 38.72 | A |
| ATOM | 1552 | CD | GLU | A | 342 | −18.825 | 100.898 | −77.973 | 1.00 | 42.11 | A |
| ATOM | 1553 | OE1 | GLU | A | 342 | −18.517 | 100.709 | −76.769 | 1.00 | 40.90 | A |
| ATOM | 1554 | OE2 | GLU | A | 342 | −18.582 | 100.058 | −78.862 | 1.00 | 44.17 | A |
| ATOM | 1555 | C | GLU | A | 342 | −20.390 | 105.064 | −78.958 | 1.00 | 34.62 | A |
| ATOM | 1556 | O | GLU | A | 342 | −20.609 | 105.087 | −80.167 | 1.00 | 33.33 | A |
| ATOM | 1557 | N | MET | A | 343 | −21.319 | 105.363 | −78.062 | 1.00 | 34.80 | A |
| ATOM | 1558 | CA | MET | A | 343 | −22.686 | 105.655 | −78.459 | 1.00 | 33.07 | A |
| ATOM | 1559 | CB | MET | A | 343 | −23.496 | 106.155 | −77.262 | 1.00 | 32.99 | A |
| ATOM | 1560 | CG | MET | A | 343 | −23.962 | 105.088 | −76.286 | 1.00 | 31.45 | A |
| ATOM | 1561 | SD | MET | A | 343 | −25.158 | 105.770 | −75.085 | 1.00 | 34.71 | A |
| ATOM | 1562 | CE | MET | A | 343 | −26.066 | 104.347 | −74.637 | 1.00 | 30.72 | A |
| ATOM | 1563 | C | MET | A | 343 | −22.823 | 106.656 | −79.593 | 1.00 | 33.70 | A |
| ATOM | 1564 | O | MET | A | 343 | −23.675 | 106.486 | −80.453 | 1.00 | 34.72 | A |
| ATOM | 1565 | N | MET | A | 344 | −21.989 | 107.693 | −79.604 | 1.00 | 32.31 | A |
| ATOM | 1566 | CA | MET | A | 344 | −22.080 | 108.728 | −80.631 | 1.00 | 31.25 | A |
| ATOM | 1567 | CB | MET | A | 344 | −21.996 | 110.091 | −79.960 | 1.00 | 30.36 | A |
| ATOM | 1568 | CG | MET | A | 344 | −22.881 | 110.196 | −78.727 | 1.00 | 30.78 | A |
| ATOM | 1569 | SD | MET | A | 344 | −23.399 | 111.855 | −78.320 | 1.00 | 33.40 | A |
| ATOM | 1570 | CE | MET | A | 344 | −22.242 | 112.378 | −77.081 | 1.00 | 27.81 | A |
| ATOM | 1571 | C | MET | A | 344 | −21.040 | 108.639 | −81.744 | 1.00 | 33.24 | A |
| ATOM | 1572 | O | MET | A | 344 | −21.198 | 109.244 | −82.811 | 1.00 | 33.06 | A |
| ATOM | 1573 | N | CYS | A | 345 | −19.970 | 107.886 | −81.491 | 1.00 | 33.58 | A |
| ATOM | 1574 | CA | CYS | A | 345 | −18.914 | 107.721 | −82.465 | 1.00 | 30.35 | A |
| ATOM | 1575 | CB | CYS | A | 345 | −17.570 | 108.071 | −81.847 | 1.00 | 29.51 | A |
| ATOM | 1576 | SG | CYS | A | 345 | −17.464 | 109.713 | −81.103 | 1.00 | 28.81 | A |
| ATOM | 1577 | C | CYS | A | 345 | −18.890 | 106.298 | −82.982 | 1.00 | 31.60 | A |
| ATOM | 1578 | O | CYS | A | 345 | −17.977 | 105.926 | −83.699 | 1.00 | 32.43 | A |
| ATOM | 1579 | N | GLY | A | 346 | −19.895 | 105.507 | −82.612 | 1.00 | 32.93 | A |
| ATOM | 1580 | CA | GLY | A | 346 | −20.002 | 104.127 | −83.068 | 1.00 | 34.64 | A |
| ATOM | 1581 | C | GLY | A | 346 | −18.800 | 103.236 | −82.826 | 1.00 | 37.57 | A |
| ATOM | 1582 | O | GLY | A | 346 | −18.863 | 102.007 | −82.976 | 1.00 | 37.29 | A |
| ATOM | 1583 | N | ARG | A | 347 | −17.705 | 103.865 | −82.417 | 1.00 | 38.88 | A |
| ATOM | 1584 | CA | ARG | A | 347 | −16.456 | 103.177 | −82.176 | 1.00 | 40.60 | A |
| ATOM | 1585 | CB | ARG | A | 347 | −15.498 | 103.503 | −83.330 | 1.00 | 42.28 | A |
| ATOM | 1586 | CG | ARG | A | 347 | −14.947 | 104.928 | −83.268 | 1.00 | 45.81 | A |
| ATOM | 1587 | CD | ARG | A | 347 | −14.730 | 105.556 | −84.635 | 1.00 | 46.25 | A |
| ATOM | 1588 | NE | ARG | A | 347 | −13.805 | 106.693 | −84.559 | 1.00 | 48.33 | A |
| ATOM | 1589 | CZ | ARG | A | 347 | −13.709 | 107.659 | −85.474 | 1.00 | 49.83 | A |
| ATOM | 1590 | NH1 | ARG | A | 347 | −14.493 | 107.647 | −86.544 | 1.00 | 48.95 | A |
| ATOM | 1591 | NH2 | ARG | A | 347 | −12.799 | 108.624 | −85.343 | 1.00 | 48.33 | A |
| ATOM | 1592 | C | ARG | A | 347 | −15.868 | 103.641 | −80.843 | 1.00 | 40.34 | A |
| ATOM | 1593 | O | ARG | A | 347 | −16.241 | 104.695 | −80.326 | 1.00 | 38.20 | A |
| ATOM | 1594 | N | LEU | A | 348 | −14.955 | 102.848 | −80.287 | 1.00 | 40.05 | A |
| ATOM | 1595 | CA | LEU | A | 348 | −14.311 | 103.206 | −79.032 | 1.00 | 40.55 | A |
| ATOM | 1596 | CB | LEU | A | 348 | −13.954 | 101.952 | −78.246 | 1.00 | 37.02 | A |
| ATOM | 1597 | CG | LEU | A | 348 | −15.058 | 101.388 | −77.357 | 1.00 | 34.76 | A |
| ATOM | 1598 | CD1 | LEU | A | 348 | −14.617 | 100.083 | −76.756 | 1.00 | 33.49 | A |
| ATOM | 1599 | CD2 | LEU | A | 348 | −15.381 | 102.376 | −76.264 | 1.00 | 33.75 | A |
| ATOM | 1600 | C | LEU | A | 348 | −13.062 | 104.017 | −79.338 | 1.00 | 42.53 | A |
| ATOM | 1601 | O | LEU | A | 348 | −12.307 | 103.680 | −80.239 | 1.00 | 42.44 | A |
| ATOM | 1602 | N | PRO | A | 349 | −12.817 | 105.088 | −78.572 | 1.00 | 45.77 | A |
| ATOM | 1603 | CD | PRO | A | 349 | −13.370 | 105.301 | −77.223 | 1.00 | 45.24 | A |
| ATOM | 1604 | CA | PRO | A | 349 | −11.639 | 105.940 | −78.801 | 1.00 | 49.88 | A |
| ATOM | 1605 | CB | PRO | A | 349 | −11.524 | 106.746 | −77.497 | 1.00 | 48.25 | A |
| ATOM | 1606 | CG | PRO | A | 349 | −12.201 | 105.884 | −76.485 | 1.00 | 46.99 | A |
| ATOM | 1607 | C | PRO | A | 349 | −10.373 | 105.153 | −79.140 | 1.00 | 53.33 | A |
| ATOM | 1608 | O | PRO | A | 349 | −9.553 | 105.585 | −79.951 | 1.00 | 53.67 | A |
| ATOM | 1609 | N | PHE | A | 350 | −10.228 | 103.994 | −78.507 | 1.00 | 57.43 | A |
| ATOM | 1610 | CA | PHE | A | 350 | −9.094 | 103.113 | −78.736 | 1.00 | 61.40 | A |
| ATOM | 1611 | CB | PHE | A | 350 | −8.243 | 102.938 | −77.470 | 1.00 | 60.86 | A |
| ATOM | 1612 | CG | PHE | A | 350 | −7.579 | 104.197 | −76.986 | 1.00 | 59.81 | A |
| ATOM | 1613 | CD1 | PHE | A | 350 | −7.126 | 104.286 | −75.669 | 1.00 | 59.25 | A |
| ATOM | 1614 | CD2 | PHE | A | 350 | −7.408 | 105.285 | −77.821 | 1.00 | 58.02 | A |
| ATOM | 1615 | CE1 | PHE | A | 350 | −6.525 | 105.437 | −75.197 | 1.00 | 57.00 | A |
| ATOM | 1616 | CE2 | PHE | A | 350 | −6.806 | 106.441 | −77.352 | 1.00 | 57.83 | A |
| ATOM | 1617 | CZ | PHE | A | 350 | −6.365 | 106.514 | −76.038 | 1.00 | 57.19 | A |
| ATOM | 1618 | C | PHE | A | 350 | −9.715 | 101.773 | −79.088 | 1.00 | 64.90 | A |
| ATOM | 1619 | O | PHE | A | 350 | −10.342 | 101.117 | −78.229 | 1.00 | 64.17 | A |
| ATOM | 1620 | N | TYR | A | 351 | −9.550 | 101.386 | −80.351 | 1.00 | 66.85 | A |
| ATOM | 1621 | CA | TYR | A | 351 | −10.071 | 100.123 | −80.857 | 1.00 | 68.83 | A |
| ATOM | 1622 | CB | TYR | A | 351 | −11.390 | 100.355 | −81.605 | 1.00 | 68.47 | A |
| ATOM | 1623 | CG | TYR | A | 351 | −11.241 | 100.959 | −82.989 | 1.00 | 68.33 | A |
| ATOM | 1624 | CD1 | TYR | A | 351 | −11.138 | 100.140 | −84.116 | 1.00 | 68.03 | A |
| ATOM | 1625 | CE1 | TYR | A | 351 | −11.017 | 100.680 | −85.394 | 1.00 | 67.30 | A |
| ATOM | 1626 | CD2 | TYR | A | 351 | −11.213 | 102.346 | −83.176 | 1.00 | 67.76 | A |
| ATOM | 1627 | CE2 | TYR | A | 351 | −11.091 | 102.904 | −84.454 | 1.00 | 66.51 | A |
| ATOM | 1628 | CZ | TYR | A | 351 | −10.997 | 102.061 | −85.562 | 1.00 | 67.80 | A |
| ATOM | 1629 | OH | TYR | A | 351 | −10.914 | 102.580 | −86.843 | 1.00 | 66.85 | A |
| ATOM | 1630 | C | TYR | A | 351 | −9.016 | 99.575 | −81.804 | 1.00 | 70.67 | A |
| ATOM | 1631 | O | TYR | A | 351 | −8.425 | 100.338 | −82.586 | 1.00 | 71.18 | A |
| ATOM | 1632 | N | ASN | A | 352 | −8.770 | 98.266 | −81.736 | 1.00 | 71.80 | A |
| ATOM | 1633 | CA | ASN | A | 352 | −7.764 | 97.642 | −82.603 | 1.00 | 72.00 | A |
| ATOM | 1634 | CB | ASN | A | 352 | −6.362 | 97.727 | −81.978 | 1.00 | 69.26 | A |
| ATOM | 1635 | CG | ASN | A | 352 | −5.705 | 99.066 | −82.185 | 1.00 | 64.92 | A |
| ATOM | 1636 | OD1 | ASN | A | 352 | −5.858 | 99.696 | −83.241 | 1.00 | 62.88 | A |
| ATOM | 1637 | ND2 | ASN | A | 352 | −4.948 | 99.509 | −81.180 | 1.00 | 62.64 | A |
| ATOM | 1638 | C | ASN | A | 352 | −7.976 | 96.188 | −82.986 | 1.00 | 72.98 | A |
| ATOM | 1639 | O | ASN | A | 352 | −9.091 | 95.663 | −83.025 | 1.00 | 73.91 | A |
| ATOM | 1640 | N | GLN | A | 353 | −6.838 | 95.565 | −83.269 | 1.00 | 73.41 | A |
| ATOM | 1641 | CA | GLN | A | 353 | −6.731 | 94.177 | −83.662 | 1.00 | 73.01 | A |
| ATOM | 1642 | CB | GLN | A | 353 | −6.023 | 94.072 | −85.024 | 1.00 | 72.45 | A |
| ATOM | 1643 | CG | GLN | A | 353 | −6.593 | 94.959 | −86.145 | 1.00 | 72.29 | A |
| ATOM | 1644 | CD | GLN | A | 353 | −5.589 | 95.194 | −87.289 | 1.00 | 71.25 | A |
| ATOM | 1645 | OE1 | GLN | A | 353 | −4.896 | 94.260 | −87.733 | 1.00 | 71.23 | A |
| ATOM | 1646 | NE2 | GLN | A | 353 | −5.515 | 96.442 | −87.777 | 1.00 | 69.34 | A |
| ATOM | 1647 | C | GLN | A | 353 | −5.858 | 93.537 | −82.583 | 1.00 | 72.33 | A |
| ATOM | 1648 | O | GLN | A | 353 | −5.051 | 92.654 | −82.879 | 1.00 | 73.66 | A |
| ATOM | 1649 | N | ASP | A | 354 | −6.007 | 93.999 | −81.340 | 1.00 | 70.59 | A |
| ATOM | 1650 | CA | ASP | A | 354 | −5.224 | 93.461 | −80.223 | 1.00 | 69.03 | A |
| ATOM | 1651 | CB | ASP | A | 354 | −3.727 | 93.585 | −80.532 | 1.00 | 67.78 | A |
| ATOM | 1652 | CG | ASP | A | 354 | −3.032 | 92.236 | −80.646 | 1.00 | 67.35 | A |
| ATOM | 1653 | OD1 | ASP | A | 354 | −3.608 | 91.310 | −81.264 | 1.00 | 67.99 | A |
| ATOM | 1654 | OD2 | ASP | A | 354 | −1.899 | 92.110 | −80.129 | 1.00 | 67.47 | A |
| ATOM | 1655 | C | ASP | A | 354 | −5.517 | 94.127 | −78.875 | 1.00 | 68.09 | A |
| ATOM | 1656 | O | ASP | A | 354 | −5.403 | 95.351 | −78.732 | 1.00 | 67.53 | A |
| ATOM | 1657 | N | HIS | A | 355 | −5.892 | 93.314 | −77.889 | 1.00 | 67.65 | A |
| ATOM | 1658 | CA | HIS | A | 355 | −6.182 | 93.816 | −76.546 | 1.00 | 67.60 | A |
| ATOM | 1659 | CB | HIS | A | 355 | −6.742 | 92.703 | −75.652 | 1.00 | 67.55 | A |
| ATOM | 1660 | CG | HIS | A | 355 | −8.210 | 92.460 | −75.826 | 1.00 | 67.24 | A |
| ATOM | 1661 | CD2 | HIS | A | 355 | −9.261 | 92.746 | −75.018 | 1.00 | 67.09 | A |
| ATOM | 1662 | ND1 | HIS | A | 355 | −8.745 | 91.871 | −76.954 | 1.00 | 67.46 | A |
| ATOM | 1663 | CE1 | HIS | A | 355 | −10.062 | 91.807 | −76.834 | 1.00 | 66.78 | A |
| ATOM | 1664 | NE2 | HIS | A | 355 | −10.400 | 92.330 | −75.668 | 1.00 | 67.42 | A |
| ATOM | 1665 | C | HIS | A | 355 | −4.908 | 94.375 | −75.920 | 1.00 | 67.46 | A |
| ATOM | 1666 | O | HIS | A | 355 | −4.875 | 95.532 | −75.501 | 1.00 | 67.39 | A |
| ATOM | 1667 | N | GLU | A | 356 | −3.864 | 93.547 | −75.856 | 1.00 | 67.04 | A |
| ATOM | 1668 | CA | GLU | A | 356 | −2.578 | 93.967 | −75.295 | 1.00 | 65.33 | A |
| ATOM | 1669 | CB | GLU | A | 356 | −1.613 | 92.765 | −75.183 | 1.00 | 63.15 | A |
| ATOM | 1670 | CG | GLU | A | 356 | −2.224 | 91.511 | −74.508 | 1.00 | 58.60 | A |
| ATOM | 1671 | CD | GLU | A | 356 | −1.198 | 90.420 | −74.123 | 1.00 | 56.10 | A |
| ATOM | 1672 | OE1 | GLU | A | 356 | −0.308 | 90.081 | −74.937 | 1.00 | 52.87 | A |
| ATOM | 1673 | OE2 | GLU | A | 356 | −1.305 | 89.879 | −72.998 | 1.00 | 53.91 | A |
| ATOM | 1674 | C | GLU | A | 356 | −2.007 | 95.040 | −76.234 | 1.00 | 64.93 | A |
| ATOM | 1675 | O | GLU | A | 356 | −1.082 | 94.783 | −77.006 | 1.00 | 63.85 | A |
| ATOM | 1676 | N | ARG | A | 357 | −2.582 | 96.241 | −76.151 | 1.00 | 63.08 | A |
| ATOM | 1677 | CA | ARG | A | 357 | −2.200 | 97.376 | −76.986 | 1.00 | 60.83 | A |
| ATOM | 1678 | CB | ARG | A | 357 | −3.046 | 97.388 | −78.259 | 1.00 | 60.63 | A |
| ATOM | 1679 | CG | ARG | A | 357 | −2.385 | 96.856 | −79.505 | 1.00 | 61.39 | A |
| ATOM | 1680 | CD | ARG | A | 357 | −3.360 | 96.895 | −80.679 | 1.00 | 60.19 | A |
| ATOM | 1681 | NE | ARG | A | 357 | −2.867 | 96.093 | −81.792 | 1.00 | 58.45 | A |
| ATOM | 1682 | CZ | ARG | A | 357 | −1.765 | 96.376 | −82.474 | 1.00 | 58.35 | A |
| ATOM | 1683 | NH1 | ARG | A | 357 | −1.054 | 97.456 | −82.147 | 1.00 | 57.33 | A |
| ATOM | 1684 | NH2 | ARG | A | 357 | −1.363 | 95.573 | −83.469 | 1.00 | 57.73 | A |
| ATOM | 1685 | C | ARG | A | 357 | −2.440 | 98.710 | −76.297 | 1.00 | 60.25 | A |
| ATOM | 1686 | O | ARG | A | 357 | −1.628 | 99.633 | −76.395 | 1.00 | 58.35 | A |
| ATOM | 1687 | N | LEU | A | 358 | −3.581 | 98.790 | −75.611 | 1.00 | 58.90 | A |
| ATOM | 1688 | CA | LEU | A | 358 | −4.036 | 100.014 | −74.958 | 1.00 | 56.98 | A |
| ATOM | 1689 | CB | LEU | A | 358 | −5.533 | 100.218 | −75.271 | 1.00 | 56.75 | A |
| ATOM | 1690 | CG | LEU | A | 358 | −6.615 | 99.341 | −74.605 | 1.00 | 54.50 | A |
| ATOM | 1691 | CD1 | LEU | A | 358 | −7.954 | 99.621 | −75.256 | 1.00 | 53.65 | A |
| ATOM | 1692 | CD2 | LEU | A | 358 | −6.270 | 97.859 | −74.722 | 1.00 | 52.82 | A |
| ATOM | 1693 | C | LEU | A | 358 | −3.820 | 100.168 | −73.462 | 1.00 | 56.02 | A |
| ATOM | 1694 | O | LEU | A | 358 | −4.244 | 101.161 | −72.884 | 1.00 | 55.19 | A |
| ATOM | 1695 | N | PHE | A | 359 | −3.162 | 99.203 | −72.835 | 1.00 | 56.21 | A |
| ATOM | 1696 | CA | PHE | A | 359 | −2.913 | 99.265 | −71.389 | 1.00 | 55.48 | A |
| ATOM | 1697 | CB | PHE | A | 359 | −2.473 | 97.885 | −70.871 | 1.00 | 58.44 | A |
| ATOM | 1698 | CG | PHE | A | 359 | −3.595 | 96.876 | −70.784 | 1.00 | 60.94 | A |
| ATOM | 1699 | CD1 | PHE | A | 359 | −4.440 | 96.648 | −71.874 | 1.00 | 62.04 | A |
| ATOM | 1700 | CD2 | PHE | A | 359 | −3.803 | 96.151 | −69.610 | 1.00 | 61.26 | A |
| ATOM | 1701 | CE1 | PHE | A | 359 | −5.482 | 95.713 | −71.793 | 1.00 | 62.67 | A |
| ATOM | 1702 | CE2 | PHE | A | 359 | −4.841 | 95.213 | −69.518 | 1.00 | 62.59 | A |
| ATOM | 1703 | CZ | PHE | A | 359 | −5.682 | 94.993 | −70.611 | 1.00 | 62.70 | A |
| ATOM | 1704 | C | PHE | A | 359 | −1.878 | 100.317 | −71.001 | 1.00 | 52.84 | A |
| ATOM | 1705 | O | PHE | A | 359 | −1.333 | 100.284 | −69.900 | 1.00 | 51.58 | A |
| ATOM | 1706 | N | GLU | A | 360 | −1.615 | 101.234 | −71.929 | 1.00 | 50.81 | A |
| ATOM | 1707 | CA | GLU | A | 360 | −0.659 | 102.329 | −71.754 | 1.00 | 48.44 | A |
| ATOM | 1708 | CB | GLU | A | 360 | 0.788 | 101.846 | −71.930 | 1.00 | 48.67 | A |
| ATOM | 1709 | CG | GLU | A | 360 | 0.945 | 100.450 | −72.495 | 1.00 | 48.17 | A |
| ATOM | 1710 | CD | GLU | A | 360 | 1.092 | 100.454 | −73.981 | 1.00 | 48.83 | A |
| ATOM | 1711 | OE1 | GLU | A | 360 | 0.429 | 99.636 | −74.642 | 1.00 | 51.50 | A |
| ATOM | 1712 | OE2 | GLU | A | 360 | 1.877 | 101.271 | −74.492 | 1.00 | 49.43 | A |
| ATOM | 1713 | C | GLU | A | 360 | −0.969 | 103.409 | −72.776 | 1.00 | 46.64 | A |
| ATOM | 1714 | O | GLU | A | 360 | −0.224 | 104.377 | −72.931 | 1.00 | 46.72 | A |
| ATOM | 1715 | N | LEU | A | 361 | −2.082 | 103.205 | −73.474 | 1.00 | 44.35 | A |
| ATOM | 1716 | CA | LEU | A | 361 | −2.595 | 104.126 | −74.475 | 1.00 | 41.50 | A |
| ATOM | 1717 | CB | LEU | A | 361 | −3.310 | 103.345 | −75.591 | 1.00 | 39.48 | A |
| ATOM | 1718 | CG | LEU | A | 361 | −2.713 | 103.296 | −77.003 | 1.00 | 36.25 | A |
| ATOM | 1719 | CD1 | LEU | A | 361 | −3.603 | 102.547 | −77.958 | 1.00 | 33.77 | A |
| ATOM | 1720 | CD2 | LEU | A | 361 | −2.562 | 104.677 | −77.505 | 1.00 | 34.78 | A |
| ATOM | 1721 | C | LEU | A | 361 | −3.596 | 105.007 | −73.725 | 1.00 | 41.11 | A |
| ATOM | 1722 | O | LEU | A | 361 | −3.715 | 106.194 | −73.984 | 1.00 | 41.16 | A |
| ATOM | 1723 | N | ILE | A | 362 | −4.306 | 104.397 | −72.783 | 1.00 | 40.61 | A |
| ATOM | 1724 | CA | ILE | A | 362 | −5.297 | 105.083 | −71.958 | 1.00 | 41.06 | A |
| ATOM | 1725 | CB | ILE | A | 362 | −6.197 | 104.061 | −71.214 | 1.00 | 39.14 | A |
| ATOM | 1726 | CG2 | ILE | A | 362 | −7.057 | 104.764 | −70.201 | 1.00 | 36.58 | A |
| ATOM | 1727 | CG1 | ILE | A | 362 | −7.049 | 103.284 | −72.213 | 1.00 | 36.16 | A |
| ATOM | 1728 | CD1 | ILE | A | 362 | −7.828 | 102.166 | −71.600 | 1.00 | 33.30 | A |
| ATOM | 1729 | C | ILE | A | 362 | −4.643 | 105.988 | −70.908 | 1.00 | 42.37 | A |
| ATOM | 1730 | O | ILE | A | 362 | −5.263 | 106.927 | −70.417 | 1.00 | 42.48 | A |
| ATOM | 1731 | N | LEU | A | 363 | −3.390 | 105.705 | −70.567 | 1.00 | 43.15 | A |
| ATOM | 1732 | CA | LEU | A | 363 | −2.682 | 106.483 | −69.560 | 1.00 | 41.88 | A |
| ATOM | 1733 | CB | LEU | A | 363 | −1.797 | 105.558 | −68.726 | 1.00 | 39.50 | A |
| ATOM | 1734 | CG | LEU | A | 363 | −2.484 | 104.738 | −67.624 | 1.00 | 38.79 | A |
| ATOM | 1735 | CD1 | LEU | A | 363 | −1.611 | 103.558 | −67.226 | 1.00 | 37.68 | A |
| ATOM | 1736 | CD2 | LEU | A | 363 | −2.779 | 105.622 | −66.417 | 1.00 | 37.32 | A |
| ATOM | 1737 | C | LEU | A | 363 | −1.850 | 107.642 | −70.088 | 1.00 | 43.44 | A |
| ATOM | 1738 | O | LEU | A | 363 | −1.427 | 108.494 | −69.313 | 1.00 | 43.50 | A |
| ATOM | 1739 | N | MET | A | 364 | −1.621 | 107.688 | −71.399 | 1.00 | 44.79 | A |
| ATOM | 1740 | CA | MET | A | 364 | −0.816 | 108.759 | −71.993 | 1.00 | 45.39 | A |
| ATOM | 1741 | CB | MET | A | 364 | 0.651 | 108.325 | −72.064 | 1.00 | 44.16 | A |
| ATOM | 1742 | CG | MET | A | 364 | 1.254 | 107.978 | −70.722 | 1.00 | 42.09 | A |
| ATOM | 1743 | SD | MET | A | 364 | 2.816 | 107.070 | −70.812 | 1.00 | 40.78 | A |
| ATOM | 1744 | CE | MET | A | 364 | 2.370 | 105.550 | −70.025 | 1.00 | 40.87 | A |
| ATOM | 1745 | C | MET | A | 364 | −1.252 | 109.230 | −73.383 | 1.00 | 46.56 | A |
| ATOM | 1746 | O | MET | A | 364 | −0.596 | 110.084 | −73.970 | 1.00 | 47.17 | A |
| ATOM | 1747 | N | GLU | A | 365 | −2.338 | 108.680 | −73.919 | 1.00 | 46.18 | A |
| ATOM | 1748 | CA | GLU | A | 365 | −2.794 | 109.096 | −75.242 | 1.00 | 46.62 | A |
| ATOM | 1749 | CB | GLU | A | 365 | −3.069 | 107.900 | −76.142 | 1.00 | 45.43 | A |
| ATOM | 1750 | CG | CLD | A | 365 | −1.974 | 107.628 | −77.148 | 1.00 | 45.51 | A |
| ATOM | 1751 | CD | GLU | A | 365 | −1.858 | 108.665 | −78.259 | 1.00 | 43.01 | A |
| ATOM | 1752 | OE1 | GLU | A | 365 | −1.281 | 109.756 | −78.042 | 1.00 | 42.64 | A |
| ATOM | 1753 | OE2 | GLU | A | 365 | −2.339 | 108.369 | −79.363 | 1.00 | 41.76 | A |
| ATOM | 1754 | C | GLU | A | 365 | −4.011 | 109.988 | −75.293 | 1.00 | 47.27 | A |
| ATOM | 1755 | O | GLU | A | 365 | −4.920 | 109.902 | −74.474 | 1.00 | 47.46 | A |
| ATOM | 1756 | N | GLU | A | 366 | −4.009 | 110.828 | −76.314 | 1.00 | 48.61 | A |
| ATOM | 1757 | CA | GLU | A | 366 | −5.065 | 111.778 | −76.574 | 1.00 | 50.19 | A |
| ATOM | 1758 | CB | GLU | A | 366 | −4.557 | 112.821 | −77.571 | 1.00 | 52.21 | A |
| ATOM | 1759 | CG | GLU | A | 366 | −3.059 | 113.108 | −77.469 | 1.00 | 52.47 | A |
| ATOM | 1760 | CD | GLU | A | 366 | −2.496 | 113.737 | −78.737 | 0.00 | 51.77 | A |
| ATOM | 1761 | OE1 | GLU | A | 366 | −2.503 | 113.069 | −79.789 | 1.00 | 50.96 | A |
| ATOM | 1762 | OE2 | GLU | A | 366 | −2.048 | 114.900 | −78.691 | 1.00 | 52.02 | A |
| ATOM | 1763 | C | GLU | A | 366 | −6.262 | 111.048 | −77.177 | 1.00 | 50.93 | A |
| ATOM | 1764 | O | GLU | A | 366 | −6.117 | 110.270 | −78.119 | 1.00 | 51.55 | A |
| ATOM | 1765 | N | ILE | A | 367 | −7.443 | 111.287 | −76.624 | 1.00 | 50.06 | A |
| ATOM | 1766 | CA | ILE | A | 367 | −8.644 | 110.672 | −77.160 | 1.00 | 49.58 | A |
| ATOM | 1767 | CB | ILE | A | 367 | −9.635 | 110.286 | −76.030 | 1.00 | 48.32 | A |
| ATOM | 1768 | CG2 | ILE | A | 367 | −11.040 | 110.168 | −76.575 | 1.00 | 48.47 | A |
| ATOM | 1769 | CG1 | ILE | A | 367 | −9.184 | 108.955 | −75.405 | 1.00 | 48.00 | A |
| ATOM | 1770 | CD1 | ILE | A | 367 | −9.969 | 108.474 | −74.187 | 1.00 | 45.21 | A |
| ATOM | 1771 | C | ILE | A | 367 | −9.239 | 111.699 | −78.124 | 1.00 | 49.83 | A |
| ATOM | 1772 | O | ILE | A | 367 | −9.702 | 112.767 | −77.715 | 1.00 | 50.38 | A |
| ATOM | 1773 | N | ARG | A | 368 | −9.171 | 111.377 | −79.415 | 1.00 | 49.10 | A |
| ATOM | 1774 | CA | ARG | A | 368 | −9.669 | 112.250 | −80.461 | 1.00 | 47.08 | A |
| ATOM | 1775 | CB | ARG | A | 368 | −8.817 | 112.101 | −81.720 | 1.00 | 48.41 | A |
| ATOM | 1776 | CG | ARG | A | 368 | −7.376 | 112.542 | −81.539 | 1.00 | 50.42 | A |
| ATOM | 1777 | CD | ARG | A | 368 | −6.605 | 112.475 | −82.844 | 0.00 | 52.02 | A |
| ATOM | 1778 | NE | ARG | A | 368 | −7.160 | 113.376 | −83.850 | 0.00 | 53.60 | A |
| ATOM | 1779 | CZ | ARG | A | 368 | −6.650 | 113.543 | −85.065 | 0.00 | 54.42 | A |
| ATOM | 1780 | NH1 | ARG | A | 368 | −5.568 | 112.868 | −85.430 | 0.00 | 54.95 | A |
| ATOM | 1781 | NH2 | ARG | A | 368 | −7.220 | 114.384 | −85.916 | 0.00 | 54.95 | A |
| ATOM | 1782 | C | ARG | A | 368 | −11.112 | 111.935 | −80.780 | 1.00 | 45.04 | A |
| ATOM | 1783 | O | ARG | A | 368 | −11.563 | 110.812 | −80.597 | 1.00 | 43.92 | A |
| ATOM | 1784 | N | PHE | A | 369 | −11.828 | 112.946 | −81.262 | 1.00 | 44.17 | A |
| ATOM | 1785 | CA | PHE | A | 369 | −13.237 | 112.822 | −81.616 | 1.00 | 42.41 | A |
| ATOM | 1786 | CB | PHE | A | 369 | −14.062 | 113.787 | −80.758 | 1.00 | 41.47 | A |
| ATOM | 1787 | CG | PHE | A | 369 | −13.627 | 113.835 | −79.325 | 1.00 | 41.31 | A |
| ATOM | 1788 | CD1 | PHE | A | 369 | −14.081 | 112.893 | −78.408 | 1.00 | 40.46 | A |
| ATOM | 1789 | CD2 | PHE | A | 369 | −12.694 | 114.773 | −78.905 | 1.00 | 40.29 | A |
| ATOM | 1790 | CE1 | PHE | A | 369 | −13.604 | 112.890 | −77.105 | 1.00 | 39.25 | A |
| ATOM | 1791 | CE2 | PHE | A | 369 | −12.213 | 114.770 | −77.597 | 1.00 | 38.36 | A |
| ATOM | 1792 | CZ | PHE | A | 369 | −12.666 | 113.834 | −76.705 | 1.00 | 38.31 | A |
| ATOM | 1793 | C | PHE | A | 369 | −13.447 | 113.156 | −83.097 | 1.00 | 42.29 | A |
| ATOM | 1794 | O | PHE | A | 369 | −12.685 | 113.916 | −83.701 | 1.00 | 42.06 | A |
| ATOM | 1795 | N | PRO | A | 370 | −14.475 | 112.571 | −83.713 | 1.00 | 42.63 | A |
| ATOM | 1796 | CD | PRO | A | 370 | −15.369 | 111.488 | −83.269 | 1.00 | 41.99 | A |
| ATOM | 1797 | CA | PRO | A | 370 | −14.689 | 112.892 | −85.120 | 1.00 | 43.64 | A |
| ATOM | 1798 | CB | PRO | A | 370 | −15.960 | 112.126 | −85.447 | 1.00 | 42.72 | A |
| ATOM | 1799 | CG | PRO | A | 370 | −15.832 | 110.907 | −84.576 | 1.00 | 42.44 | A |
| ATOM | 1800 | C | PRO | A | 370 | −14.878 | 114.395 | −85.258 | 1.00 | 45.92 | A |
| ATOM | 1801 | O | PRO | A | 370 | −14.827 | 115.127 | −84.277 | 1.00 | 45.82 | A |
| ATOM | 1802 | N | ARG | A | 371 | −15.083 | 114.861 | −86.481 | 1.00 | 49.54 | A |
| ATOM | 1803 | CA | ARG | A | 371 | −15.319 | 116.282 | −86.712 | 1.00 | 50.65 | A |
| ATOM | 1804 | CB | ARG | A | 371 | −14.985 | 116.649 | −88.161 | 1.00 | 51.72 | A |
| ATOM | 1805 | CG | ARG | A | 371 | −14.061 | 115.656 | −88.873 | 1.00 | 52.39 | A |
| ATOM | 1806 | CD | ARG | A | 371 | −13.701 | 116.170 | −90.271 | 1.00 | 54.02 | A |
| ATOM | 1807 | NE | ARG | A | 371 | −13.275 | 115.105 | −91.175 | 1.00 | 54.49 | A |
| ATOM | 1808 | CZ | ARG | A | 371 | −14.101 | 114.351 | −91.903 | 1.00 | 53.61 | A |
| ATOM | 1809 | NH1 | ARG | A | 371 | −15.420 | 114.539 | −91.850 | 1.00 | 52.26 | A |
| ATOM | 1810 | NH2 | ARG | A | 371 | −13.600 | 113.398 | −92.679 | 1.00 | 52.07 | A |
| ATOM | 1811 | C | ARG | A | 371 | −16.817 | 116.447 | −86.456 | 1.00 | 51.39 | A |
| ATOM | 1812 | O | ARG | A | 371 | −17.257 | 117.411 | −85.828 | 1.00 | 51.37 | A |
| ATOM | 1813 | N | THR | A | 372 | −17.570 | 115.459 | −86.941 | 1.00 | 50.88 | A |
| ATOM | 1814 | CA | THR | A | 372 | −19.023 | 115.356 | −86.818 | 1.00 | 50.83 | A |
| ATOM | 1815 | CB | THR | A | 372 | −19.507 | 114.071 | −87.438 | 1.00 | 50.23 | A |
| ATOM | 1816 | OG1 | THR | A | 372 | −18.401 | 113.414 | −88.068 | 1.00 | 49.03 | A |
| ATOM | 1817 | CG2 | THR | A | 372 | −20.627 | 114.346 | −88.417 | 1.00 | 49.67 | A |
| ATOM | 1818 | C | THR | A | 372 | −19.545 | 115.311 | −85.382 | 1.00 | 51.79 | A |
| ATOM | 1819 | O | THR | A | 372 | −20.655 | 114.818 | −85.136 | 1.00 | 51.82 | A |
| ATOM | 1820 | N | LEU | A | 373 | −18.753 | 115.791 | −84.434 | 1.00 | 50.84 | A |
| ATOM | 1821 | CA | LEU | A | 373 | −19.169 | 115.762 | −83.047 | 1.00 | 48.92 | A |
| ATOM | 1822 | CB | LEU | A | 373 | −18.029 | 115.247 | −82.166 | 1.00 | 47.97 | A |
| ATOM | 1823 | CG | LEU | A | 373 | −18.446 | 114.519 | −80.889 | 1.00 | 46.19 | A |
| ATOM | 1824 | CD1 | LEU | A | 373 | −19.127 | 113.208 | −81.242 | 1.00 | 45.21 | A |
| ATOM | 1825 | CD2 | LEU | A | 373 | −17.224 | 114.266 | −80.038 | 1.00 | 47.17 | A |
| ATOM | 1826 | C | LEU | A | 373 | −19.540 | 117.166 | −82.647 | 1.00 | 48.09 | A |
| ATOM | 1827 | O | LEU | A | 373 | −18.676 | 118.040 | −82.602 | 1.00 | 48.56 | A |
| ATOM | 1828 | N | SER | A | 374 | −20.823 | 117.379 | −82.367 | 1.00 | 47.93 | A |
| ATOM | 1829 | CA | SER | A | 374 | −21.308 | 118.691 | −81.967 | 1.00 | 49.03 | A |
| ATOM | 1830 | CB | SER | A | 374 | −22.617 | 118.581 | −81.193 | 1.00 | 47.65 | A |
| ATOM | 1831 | OG | SER | A | 374 | −22.382 | 118.167 | −79.858 | 1.00 | 46.63 | A |
| ATOM | 1832 | C | SER | A | 374 | −20.268 | 119.332 | −81.073 | 1.00 | 50.48 | A |
| ATOM | 1833 | O | SER | A | 374 | −19.558 | 118.647 | −80.341 | 1.00 | 50.96 | A |
| ATOM | 1834 | N | PRO | A | 375 | −20.152 | 120.662 | −81.130 | 1.00 | 51.38 | A |
| ATOM | 1835 | CD | PRO | A | 375 | −20.942 | 121.628 | −81.915 | 1.00 | 50.30 | A |
| ATOM | 1836 | CA | PRO | A | 375 | −19.165 | 121.337 | −80.284 | 1.00 | 51.78 | A |
| ATOM | 1837 | CB | PRO | A | 375 | −19.373 | 122.815 | −80.622 | 1.00 | 51.95 | A |
| ATOM | 1838 | CG | PRO | A | 375 | −20.824 | 122.876 | −81.081 | 1.00 | 51.37 | A |
| ATOM | 1839 | C | PRO | A | 375 | −19.372 | 121.021 | −78.804 | 1.00 | 51.12 | A |
| ATOM | 1840 | O | PRO | A | 375 | −18.418 | 120.716 | −78.087 | 1.00 | 50.68 | A |
| ATOM | 1841 | N | GLU | A | 376 | −20.622 | 121.077 | −78.355 | 1.00 | 50.44 | A |
| ATOM | 1842 | CA | GLU | A | 376 | −20.940 | 120.796 | −76.959 | 1.00 | 50.98 | A |
| ATOM | 1843 | CB | GLU | A | 376 | −22.413 | 121.107 | −76.682 | 1.00 | 51.55 | A |
| ATOM | 1844 | CG | GLU | A | 376 | −22.744 | 122.572 | −76.933 | 1.00 | 56.21 | A |
| ATOM | 1845 | CD | GLU | A | 376 | −24.226 | 122.900 | −76.787 | 1.00 | 57.71 | A |
| ATOM | 1846 | OE1 | GLU | A | 376 | −25.057 | 122.171 | −77.405 | 1.00 | 58.41 | A |
| ATOM | 1847 | OE2 | GLU | A | 376 | −24.554 | 123.889 | −76.069 | 1.00 | 57.40 | A |
| ATOM | 1848 | C | GLU | A | 376 | −20.611 | 119.359 | −76.571 | 1.00 | 50.65 | A |
| ATOM | 1849 | O | GLU | A | 376 | −19.955 | 119.130 | −75.555 | 1.00 | 50.30 | A |
| ATOM | 1850 | N | ALA | A | 377 | −21.047 | 118.399 | −77.388 | 1.00 | 50.04 | A |
| ATOM | 1851 | CA | ALA | A | 377 | −20.799 | 116.978 | −77.131 | 1.00 | 49.23 | A |
| ATOM | 1852 | CB | ALA | A | 377 | −21.682 | 116.138 | −78.033 | 1.00 | 49.05 | A |
| ATOM | 1853 | C | ALA | A | 377 | −19.323 | 116.557 | −77.285 | 1.00 | 49.02 | A |
| ATOM | 1854 | O | ALA | A | 377 | −18.966 | 115.396 | −77.094 | 1.00 | 47.67 | A |
| ATOM | 1855 | N | LYS | A | 378 | −18.470 | 117.511 | −77.630 | 1.00 | 49.27 | A |
| ATOM | 1856 | CA | LYS | A | 378 | −17.048 | 117.247 | −77.785 | 1.00 | 49.40 | A |
| ATOM | 1857 | CB | LYS | A | 378 | −16.505 | 117.963 | −79.026 | 1.00 | 50.09 | A |
| ATOM | 1858 | CG | LYS | A | 378 | −15.056 | 117.632 | −79.344 | 1.00 | 51.44 | A |
| ATOM | 1859 | CD | LYS | A | 378 | −14.620 | 118.251 | −80.660 | 1.00 | 52.50 | A |
| ATOM | 1860 | CE | LYS | A | 378 | −13.164 | 117.926 | −80.974 | 1.00 | 52.00 | A |
| ATOM | 1861 | NZ | LYS | A | 378 | −12.726 | 118.550 | −82.250 | 1.00 | 49.98 | A |
| ATOM | 1862 | C | LYS | A | 378 | −16.380 | 117.777 | −76.528 | 1.00 | 48.68 | A |
| ATOM | 1863 | O | LYS | A | 378 | −15.265 | 117.393 | −76.185 | 1.00 | 48.48 | A |
| ATOM | 1864 | N | SER | A | 379 | −17.092 | 118.670 | −75.847 | 1.00 | 48.49 | A |
| ATOM | 1865 | CA | SER | A | 379 | −16.625 | 119.267 | −74.599 | 1.00 | 46.75 | A |
| ATOM | 1866 | CB | SER | A | 379 | −17.388 | 120.554 | −74.309 | 1.00 | 44.14 | A |
| ATOM | 1867 | OG | SER | A | 379 | −16.817 | 121.223 | −73.209 | 1.00 | 39.06 | A |
| ATOM | 1868 | C | SER | A | 379 | −16.911 | 118.249 | −73.505 | 1.00 | 46.36 | A |
| ATOM | 1869 | O | SER | A | 379 | −16.113 | 118.060 | −72.580 | 1.00 | 44.88 | A |
| ATOM | 1870 | N | LEU | A | 380 | −18.066 | 117.597 | −73.635 | 1.00 | 45.53 | A |
| ATOM | 1871 | CA | LEU | A | 380 | −18.493 | 116.565 | −72.706 | 1.00 | 44.33 | A |
| ATOM | 1872 | CB | LEU | A | 380 | −19.820 | 115.966 | −73.162 | 1.00 | 42.39 | A |
| ATOM | 1873 | CG | LEU | A | 380 | −20.356 | 114.907 | −72.204 | 1.00 | 41.20 | A |
| ATOM | 1874 | CD1 | LEU | A | 380 | −20.698 | 115.566 | −70.863 | 1.00 | 39.88 | A |
| ATOM | 1875 | CD2 | LEU | A | 380 | −21.565 | 114.222 | −72.809 | 1.00 | 40.47 | A |
| ATOM | 1876 | C | LEU | A | 380 | −17.449 | 115.460 | −72.664 | 1.00 | 44.77 | A |
| ATOM | 1877 | O | LEU | A | 380 | −16.691 | 115.337 | −71.701 | 1.00 | 44.52 | A |
| ATOM | 1878 | N | LEU | A | 381 | −17.433 | 114.658 | −73.729 | 1.00 | 44.80 | A |
| ATOM | 1879 | CA | LEU | A | 381 | −16.505 | 113.533 | −73.883 | 1.00 | 44.13 | A |
| ATOM | 1880 | CB | LEU | A | 381 | −16.516 | 113.052 | −75.336 | 1.00 | 43.54 | A |
| ATOM | 1881 | CG | LEU | A | 381 | −17.851 | 112.475 | −75.809 | 1.00 | 44.44 | A |
| ATOM | 1882 | CD1 | LEU | A | 381 | −17.940 | 112.490 | −77.315 | 1.00 | 42.79 | A |
| ATOM | 1883 | CD2 | LEU | A | 381 | −17.999 | 111.072 | −75.263 | 1.00 | 45.30 | A |
| ATOM | 1884 | C | LEU | A | 381 | −15.081 | 113.881 | −73.476 | 1.00 | 43.00 | A |
| ATOM | 1885 | O | LEU | A | 381 | −14.385 | 113.081 | −72.865 | 1.00 | 42.58 | A |
| ATOM | 1886 | N | ALA | A | 382 | −14.651 | 115.083 | −73.817 | 1.00 | 42.31 | A |
| ATOM | 1887 | CA | ALA | A | 382 | −13.312 | 115.504 | −73.483 | 1.00 | 41.97 | A |
| ATOM | 1888 | CB | ALA | A | 382 | −12.993 | 116.797 | −74.195 | 1.00 | 42.13 | A |
| ATOM | 1889 | C | ALA | A | 382 | −13.168 | 115.683 | −71.985 | 1.00 | 42.03 | A |
| ATOM | 1890 | O | ALA | A | 382 | −12.075 | 115.557 | −71.449 | 1.00 | 42.73 | A |
| ATOM | 1891 | N | GLY | A | 383 | −14.278 | 115.975 | −71.314 | 1.00 | 42.56 | A |
| ATOM | 1892 | CA | GLY | A | 383 | −14.254 | 116.199 | −69.876 | 1.00 | 41.18 | A |
| ATOM | 1893 | C | GLY | A | 383 | −14.493 | 114.964 | −69.032 | 1.00 | 39.86 | A |
| ATOM | 1894 | O | GLY | A | 383 | −13.974 | 114.861 | −67.920 | 1.00 | 37.66 | A |
| ATOM | 1895 | N | LEU | A | 384 | −15.299 | 114.042 | −69.556 | 1.00 | 38.27 | A |
| ATOM | 1896 | CA | LEU | A | 384 | −15.593 | 112.799 | −68.866 | 1.00 | 38.05 | A |
| ATOM | 1897 | CB | LEU | A | 384 | −16.868 | 112.163 | −69.410 | 1.00 | 36.40 | A |
| ATOM | 1898 | CG | LEU | A | 384 | −18.208 | 112.776 | −68.988 | 1.00 | 36.16 | A |
| ATOM | 1899 | CD1 | LEU | A | 384 | −19.354 | 112.028 | −69.666 | 1.00 | 35.25 | A |
| ATOM | 1900 | CD2 | LEU | A | 384 | −18.353 | 112.711 | −67.471 | 1.00 | 33.07 | A |
| ATOM | 1901 | C | LEU | A | 384 | −14.435 | 111.837 | −69.053 | 1.00 | 38.84 | A |
| ATOM | 1902 | O | LEU | A | 384 | −14.378 | 110.793 | −68.413 | 1.00 | 41.26 | A |
| ATOM | 1903 | N | LEU | A | 385 | −13.507 | 112.199 | −69.933 | 1.00 | 38.54 | A |
| ATOM | 1904 | CA | LEU | A | 385 | −12.338 | 111.375 | −70.213 | 1.00 | 37.80 | A |
| ATOM | 1905 | CB | LEU | A | 385 | −12.260 | 111.037 | −71.699 | 1.00 | 34.89 | A |
| ATOM | 1906 | CG | LEU | A | 385 | −13.354 | 110.156 | −72.283 | 1.00 | 34.69 | A |
| ATOM | 1907 | CD1 | LEU | A | 385 | −13.139 | 110.037 | −73.769 | 1.00 | 36.09 | A |
| ATOM | 1908 | CD2 | LEU | A | 385 | −13.327 | 108.785 | −71.646 | 1.00 | 35.52 | A |
| ATOM | 1909 | C | LEU | A | 385 | −11.024 | 112.015 | −69.797 | 1.00 | 39.02 | A |
| ATOM | 1910 | O | LEU | A | 385 | −9.970 | 111.437 | −70.031 | 1.00 | 39.50 | A |
| ATOM | 1911 | N | LYS | A | 386 | −11.068 | 113.204 | −69.199 | 1.00 | 41.43 | A |
| ATOM | 1912 | CA | LYS | A | 386 | −9.831 | 113.851 | −68.778 | 1.00 | 42.54 | A |
| ATOM | 1913 | CB | LYS | A | 386 | −10.099 | 115.209 | −68.122 | 1.00 | 42.65 | A |
| ATOM | 1914 | CG | LYS | A | 386 | −10.494 | 116.285 | −69.131 | 1.00 | 45.71 | A |
| ATOM | 1915 | CD | LYS | A | 386 | −10.054 | 117.685 | −68.694 | 1.00 | 48.93 | A |
| ATOM | 1916 | CE | LYS | A | 386 | −10.357 | 118.770 | −69.756 | 1.00 | 48.50 | A |
| ATOM | 1917 | NZ | LYS | A | 386 | −11.811 | 119.053 | −69.985 | 1.00 | 47.77 | A |
| ATOM | 1918 | C | LYS | A | 386 | −9.158 | 112.902 | −67.815 | 1.00 | 42.73 | A |
| ATOM | 1919 | O | LYS | A | 386 | −9.711 | 112.575 | −66.768 | 1.00 | 42.71 | A |
| ATOM | 1920 | N | LYS | A | 387 | −7.969 | 112.448 | −68.197 | 1.00 | 42.54 | A |
| ATOM | 1921 | CA | LYS | A | 387 | −7.199 | 111.491 | −67.414 | 1.00 | 43.21 | A |
| ATOM | 1922 | CB | LYS | A | 387 | −5.786 | 111.387 | −67.975 | 1.00 | 43.49 | A |
| ATOM | 1923 | CG | LYS | A | 387 | −5.747 | 111.162 | −69.476 | 1.00 | 41.60 | A |
| ATOM | 1924 | CD | LYS | A | 387 | −6.243 | 109.800 | −69.888 | 1.00 | 40.78 | A |
| ATOM | 1925 | CE | LYS | A | 387 | −6.094 | 109.644 | −71.390 | 1.00 | 41.72 | A |
| ATOM | 1926 | NZ | LYS | A | 387 | −4.809 | 110.242 | −71.861 | 1.00 | 38.93 | A |
| ATOM | 1927 | C | LYS | A | 387 | −7.141 | 111.766 | −65.922 | 1.00 | 43.35 | A |
| ATOM | 1928 | O | LYS | A | 387 | −7.398 | 110.867 | −65.123 | 1.00 | 44.75 | A |
| ATOM | 1929 | N | ASP | A | 388 | −6.789 | 112.989 | −65.540 | 1.00 | 43.54 | A |
| ATOM | 1930 | CA | ASP | A | 388 | −6.726 | 113.335 | −64.125 | 1.00 | 44.15 | A |
| ATOM | 1931 | CB | ASP | A | 388 | −6.209 | 114.757 | −63.951 | 1.00 | 43.62 | A |
| ATOM | 1932 | CG | ASP | A | 388 | −5.635 | 115.005 | −62.576 | 1.00 | 43.61 | A |
| ATOM | 1933 | OD1 | ASP | A | 388 | −6.302 | 114.690 | −61.570 | 1.00 | 42.68 | A |
| ATOM | 1934 | OD2 | ASP | A | 388 | −4.507 | 115.530 | −62.505 | 1.00 | 44.42 | A |
| ATOM | 1935 | C | ASP | A | 388 | −8.155 | 113.247 | −63.614 | 1.00 | 45.46 | A |
| ATOM | 1936 | O | ASP | A | 388 | −9.056 | 113.825 | −64.212 | 1.00 | 46.61 | A |
| ATOM | 1937 | N | PRO | A | 389 | −8.391 | 112.531 | −62.500 | 1.00 | 46.10 | A |
| ATOM | 1938 | CD | PRO | A | 389 | −7.468 | 111.951 | −61.506 | 1.00 | 44.92 | A |
| ATOM | 1939 | CA | PRO | A | 389 | −9.767 | 112.448 | −62.023 | 1.00 | 46.39 | A |
| ATOM | 1940 | CB | PRO | A | 389 | −9.688 | 111.364 | −60.965 | 1.00 | 44.37 | A |
| ATOM | 1941 | CG | PRO | A | 389 | −8.381 | 111.657 | −60.336 | 1.00 | 45.26 | A |
| ATOM | 1942 | C | PRO | A | 389 | −10.209 | 113.783 | −61.460 | 1.00 | 47.88 | A |
| ATOM | 1943 | O | PRO | A | 389 | −11.277 | 114.278 | −61.799 | 1.00 | 48.62 | A |
| ATOM | 1944 | N | LYS | A | 390 | −9.365 | 114.368 | −60.617 | 1.00 | 48.64 | A |
| ATOM | 1945 | CA | LYS | A | 390 | −9.655 | 115.649 | −59.991 | 1.00 | 49.76 | A |
| ATOM | 1946 | CB | LYS | A | 390 | −8.406 | 116.176 | −59.279 | 1.00 | 51.91 | A |
| ATOM | 1947 | CG | LYS | A | 390 | −8.097 | 115.471 | −57.957 | 1.00 | 51.28 | A |
| ATOM | 1948 | CD | LYS | A | 390 | −6.665 | 115.718 | −57.521 | 1.00 | 51.94 | A |
| ATOM | 1949 | CE | LYS | A | 390 | −6.346 | 114.994 | −56.220 | 1.00 | 52.72 | A |
| ATOM | 1950 | NZ | LYS | A | 390 | −4.869 | 114.972 | −55.948 | 1.00 | 52.27 | A |
| ATOM | 1951 | C | LYS | A | 390 | −10.136 | 116.661 | −61.010 | 1.00 | 50.10 | A |
| ATOM | 1952 | O | LYS | A | 390 | −10.845 | 117.610 | −60.674 | 1.00 | 50.03 | A |
| ATOM | 1953 | N | GLN | A | 391 | −9.756 | 116.441 | −62.262 | 1.00 | 50.17 | A |
| ATOM | 1954 | CA | GLN | A | 391 | −10.136 | 117.324 | −63.354 | 1.00 | 49.69 | A |
| ATOM | 1955 | CB | GLN | A | 391 | −8.929 | 117.548 | −64.261 | 1.00 | 49.67 | A |
| ATOM | 1956 | CG | GLN | A | 391 | −8.031 | 118.674 | −63.801 | 0.00 | 49.20 | A |
| ATOM | 1957 | CD | GLN | A | 391 | −6.719 | 118.695 | −64.543 | 0.00 | 48.71 | A |
| ATOM | 1958 | OE1 | GLN | A | 391 | −6.678 | 118.462 | −65.758 | 1.00 | 47.00 | A |
| ATOM | 1959 | NE2 | GLN | A | 391 | −5.634 | 118.984 | −63.821 | 1.00 | 48.83 | A |
| ATOM | 1960 | C | GLN | A | 391 | −11.332 | 116.834 | −64.176 | 1.00 | 49.45 | A |
| ATOM | 1961 | O | GLN | A | 391 | −12.016 | 117.630 | −64.820 | 1.00 | 50.24 | A |
| ATOM | 1962 | N | ARG | A | 392 | −11.589 | 115.530 | −64.138 | 1.00 | 48.09 | A |
| ATOM | 1963 | CA | ARG | A | 392 | −12.695 | 114.929 | −64.878 | 1.00 | 47.20 | A |
| ATOM | 1964 | CB | ARG | A | 392 | −12.757 | 113.441 | −64.539 | 1.00 | 46.04 | A |
| ATOM | 1965 | CG | ARG | A | 392 | −13.458 | 112.560 | −65.561 | 1.00 | 45.97 | A |
| ATOM | 1966 | CD | ARG | A | 392 | −13.185 | 111.099 | −65.259 | 1.00 | 44.98 | A |
| ATOM | 1967 | NE | ARG | A | 392 | −11.764 | 110.884 | −64.995 | 1.00 | 44.47 | A |
| ATOM | 1968 | CZ | ARG | A | 392 | −11.241 | 109.756 | −64.527 | 1.00 | 43.95 | A |
| ATOM | 1969 | NH1 | ARG | A | 392 | −12.014 | 108.717 | −64.266 | 1.00 | 43.76 | A |
| ATOM | 1970 | NH2 | ARG | A | 392 | −9.939 | 109.671 | −64.311 | 1.00 | 43.88 | A |
| ATOM | 1971 | C | ARG | A | 392 | −14.026 | 115.606 | −64.538 | 1.00 | 46.76 | A |
| ATOM | 1972 | O | ARG | A | 392 | −14.095 | 116.386 | −63.588 | 1.00 | 47.44 | A |
| ATOM | 1973 | N | LEU | A | 393 | −15.070 | 115.332 | −65.328 | 1.00 | 46.23 | A |
| ATOM | 1974 | CA | LEU | A | 393 | −16.414 | 115.883 | −65.074 | 1.00 | 44.99 | A |
| ATOM | 1975 | CB | LEU | A | 393 | −17.281 | 115.834 | −66.326 | 1.00 | 43.19 | A |
| ATOM | 1976 | CG | LEU | A | 393 | −17.276 | 117.067 | −67.208 | 1.00 | 41.49 | A |
| ATOM | 1977 | CD1 | LEU | A | 393 | −18.281 | 116.891 | −68.328 | 1.00 | 40.13 | A |
| ATOM | 1978 | CD2 | LEU | A | 393 | −17.620 | 118.270 | −66.361 | 1.00 | 42.02 | A |
| ATOM | 1979 | C | LEU | A | 393 | −17.080 | 115.042 | −63.994 | 1.00 | 45.79 | A |
| ATOM | 1980 | O | LEU | A | 393 | −17.734 | 114.046 | −64.276 | 1.00 | 45.67 | A |
| ATOM | 1981 | N | GLY | A | 394 | −16.920 | 115.468 | −62.753 | 1.00 | 46.54 | A |
| ATOM | 1982 | CA | GLY | A | 394 | −17.449 | 114.716 | −61.639 | 1.00 | 46.91 | A |
| ATOM | 1983 | C | GLY | A | 394 | −16.221 | 114.425 | −60.803 | 1.00 | 47.02 | A |
| ATOM | 1984 | O | GLY | A | 394 | −16.155 | 113.465 | −60.048 | 1.00 | 46.57 | A |
| ATOM | 1985 | N | GLY | A | 395 | −15.219 | 115.270 | −60.988 | 1.00 | 47.22 | A |
| ATOM | 1986 | CA | GLY | A | 395 | −13.994 | 115.139 | −60.241 | 1.00 | 49.50 | A |
| ATOM | 1987 | C | GLY | A | 395 | −14.081 | 116.170 | −59.144 | 1.00 | 50.85 | A |
| ATOM | 1988 | O | GLY | A | 395 | −13.344 | 116.118 | −58.156 | 1.00 | 53.00 | A |
| ATOM | 1989 | N | GLY | A | 396 | −15.000 | 117.113 | −59.329 | 1.00 | 50.83 | A |
| ATOM | 1990 | CA | GLY | A | 396 | −15.201 | 118.162 | −58.349 | 1.00 | 50.44 | A |
| ATOM | 1991 | C | GLY | A | 396 | −16.000 | 117.677 | −57.153 | 1.00 | 50.19 | A |
| ATOM | 1992 | O | GLY | A | 396 | −16.692 | 116.656 | −57.235 | 1.00 | 50.51 | A |
| ATOM | 1993 | N | PRO | A | 397 | −15.937 | 118.405 | −56.026 | 1.00 | 49.00 | A |
| ATOM | 1994 | CD | PRO | A | 397 | −15.287 | 119.722 | −55.909 | 0.00 | 48.69 | A |
| ATOM | 1995 | CA | PRO | A | 397 | −16.645 | 118.071 | −54.788 | 1.00 | 48.22 | A |
| ATOM | 1996 | CB | PRO | A | 397 | −16.269 | 119.224 | −53.860 | 1.00 | 47.31 | A |
| ATOM | 1997 | CG | PRO | A | 397 | −16.083 | 120.366 | −54.804 | 0.00 | 48.31 | A |
| ATOM | 1998 | C | PRO | A | 397 | −18.159 | 117.906 | −54.938 | 1.00 | 47.36 | A |
| ATOM | 1999 | O | PRO | A | 397 | −18.786 | 117.199 | −54.149 | 1.00 | 47.61 | A |
| ATOM | 2000 | N | SER | A | 398 | −18.731 | 118.545 | −55.961 | 1.00 | 45.23 | A |
| ATOM | 2001 | CA | SER | A | 398 | −20.169 | 118.495 | −56.227 | 1.00 | 42.61 | A |
| ATOM | 2002 | CB | SER | A | 398 | −20.608 | 119.771 | −56.919 | 1.00 | 43.28 | A |
| ATOM | 2003 | OG | SER | A | 398 | −19.939 | 119.905 | −58.155 | 1.00 | 44.50 | A |
| ATOM | 2004 | C | SER | A | 398 | −20.621 | 117.311 | −57.071 | 1.00 | 41.14 | A |
| ATOM | 2005 | O | SER | A | 398 | −21.785 | 117.227 | −57.439 | 1.00 | 39.80 | A |
| ATOM | 2006 | N | ASP | A | 399 | −19.695 | 116.410 | −57.386 | 1.00 | 39.71 | A |
| ATOM | 2007 | CA | ASP | A | 399 | −19.991 | 115.210 | −58.166 | 1.00 | 38.56 | A |
| ATOM | 2008 | CB | ASP | A | 399 | −20.703 | 114.168 | −57.286 | 1.00 | 38.01 | A |
| ATOM | 2009 | CG | ASP | A | 399 | −20.402 | 112.733 | −57.711 | 1.00 | 38.48 | A |
| ATOM | 2010 | OD1 | ASP | A | 399 | −21.315 | 112.038 | −58.193 | 1.00 | 38.28 | A |
| ATOM | 2011 | OD2 | ASP | A | 399 | −19.244 | 112.289 | −57.560 | 1.00 | 38.15 | A |
| ATOM | 2012 | C | ASP | A | 399 | −20.799 | 115.446 | −59.446 | 1.00 | 37.24 | A |
| ATOM | 2013 | O | ASP | A | 399 | −20.451 | 116.293 | −60.249 | 1.00 | 37.13 | A |
| ATOM | 2014 | N | ALA | A | 400 | −21.878 | 114.699 | −59.629 | 1.00 | 34.67 | A |
| ATOM | 2015 | CA | ALA | A | 400 | −22.684 | 114.804 | −60.837 | 1.00 | 35.08 | A |
| ATOM | 2016 | CB | ALA | A | 400 | −23.905 | 113.910 | −60.709 | 1.00 | 33.43 | A |
| ATOM | 2017 | C | ALA | A | 400 | −23.106 | 116.209 | −61.287 | 1.00 | 36.56 | A |
| ATOM | 2018 | O | ALA | A | 400 | −23.312 | 116.444 | −62.480 | 1.00 | 36.63 | A |
| ATOM | 2019 | N | LYS | A | 401 | −23.234 | 117.142 | −60.348 | 1.00 | 35.88 | A |
| ATOM | 2020 | CA | LYS | A | 401 | −23.645 | 118.494 | −60.698 | 1.00 | 34.95 | A |
| ATOM | 2021 | CB | LYS | A | 401 | −23.628 | 119.397 | −59.472 | 1.00 | 32.50 | A |
| ATOM | 2022 | CG | LYS | A | 401 | −24.708 | 119.078 | −58.484 | 1.00 | 31.43 | A |
| ATOM | 2023 | CD | LYS | A | 401 | −24.507 | 119.886 | −57.246 | 1.00 | 28.22 | A |
| ATOM | 2024 | CE | LYS | A | 401 | −25.278 | 119.318 | −56.089 | 1.00 | 27.81 | A |
| ATOM | 2025 | NZ | LYS | A | 401 | −24.727 | 119.867 | −54.830 | 1.00 | 25.14 | A |
| ATOM | 2026 | C | LYS | A | 401 | −22.791 | 119.106 | −61.785 | 1.00 | 34.81 | A |
| ATOM | 2027 | O | LYS | A | 401 | −23.160 | 120.125 | −62.363 | 1.00 | 36.29 | A |
| ATOM | 2028 | N | GLU | A | 402 | −21.650 | 118.492 | −62.063 | 1.00 | 34.06 | A |
| ATOM | 2029 | CA | GLU | A | 402 | −20.768 | 118.993 | −63.104 | 1.00 | 35.31 | A |
| ATOM | 2030 | CB | GLU | A | 402 | −19.320 | 118.574 | −62.844 | 1.00 | 35.15 | A |
| ATOM | 2031 | CG | GLU | A | 402 | −18.942 | 118.522 | −61.390 | 1.00 | 35.78 | A |
| ATOM | 2032 | CD | GLU | A | 402 | −17.607 | 119.159 | −61.097 | 1.00 | 36.09 | A |
| ATOM | 2033 | OE1 | GLU | A | 402 | −16.573 | 118.719 | −61.635 | 1.00 | 35.51 | A |
| ATOM | 2034 | OE2 | GLU | A | 402 | −17.594 | 120.114 | −60.302 | 1.00 | 37.27 | A |
| ATOM | 2035 | C | GLU | A | 402 | −21.225 | 118.403 | −64.425 | 1.00 | 36.16 | A |
| ATOM | 2036 | O | GLU | A | 402 | −21.097 | 119.024 | −65.472 | 1.00 | 37.83 | A |
| ATOM | 2037 | N | VAL | A | 403 | −21.775 | 117.196 | −64.351 | 1.00 | 36.13 | A |
| ATOM | 2038 | CA | VAL | A | 403 | −22.243 | 116.465 | −65.520 | 1.00 | 35.96 | A |
| ATOM | 2039 | CB | VAL | A | 403 | −22.195 | 114.948 | −65.240 | 1.00 | 32.70 | A |
| ATOM | 2040 | CG1 | VAL | A | 403 | −22.532 | 114.164 | −66.468 | 1.00 | 30.74 | A |
| ATOM | 2041 | CG2 | VAL | A | 403 | −20.826 | 114.569 | −64.768 | 1.00 | 32.27 | A |
| ATOM | 2042 | C | VAL | A | 403 | −23.652 | 116.873 | −65.932 | 1.00 | 38.49 | A |
| ATOM | 2043 | O | VAL | A | 403 | −23.971 | 116.923 | −67.122 | 1.00 | 40.23 | A |
| ATOM | 2044 | N | MET | A | 404 | −24.482 | 117.180 | −64.938 | 1.00 | 40.98 | A |
| ATOM | 2045 | CA | MET | A | 404 | −25.873 | 117.589 | −65.145 | 1.00 | 40.56 | A |
| ATOM | 2046 | CB | MET | A | 404 | −26.629 | 117.556 | −63.825 | 1.00 | 38.94 | A |
| ATOM | 2047 | CG | MET | A | 404 | −26.923 | 116.169 | −63.340 | 1.00 | 38.99 | A |
| ATOM | 2048 | SD | MET | A | 404 | −27.808 | 116.211 | −61.823 | 1.00 | 36.34 | A |
| ATOM | 2049 | CE | MET | A | 404 | −26.589 | 116.901 | −60.805 | 1.00 | 37.24 | A |
| ATOM | 2050 | C | MET | A | 404 | −26.001 | 118.974 | −65.720 | 1.00 | 41.72 | A |
| ATOM | 2051 | O | MET | A | 404 | −26.781 | 119.205 | −66.635 | 1.00 | 42.27 | A |
| ATOM | 2052 | N | GLU | A | 405 | −25.240 | 119.900 | −65.160 | 1.00 | 43.19 | A |
| ATOM | 2053 | CA | GLU | A | 405 | −25.278 | 121.277 | −65.608 | 1.00 | 45.78 | A |
| ATOM | 2054 | CB | GLU | A | 405 | −25.012 | 122.207 | −64.425 | 1.00 | 46.91 | A |
| ATOM | 2055 | CG | GLU | A | 405 | −26.226 | 122.431 | −63.550 | 1.00 | 48.97 | A |
| ATOM | 2056 | CD | GLU | A | 405 | −25.867 | 122.541 | −62.086 | 1.00 | 48.81 | A |
| ATOM | 2057 | OE1 | GLU | A | 405 | −24.936 | 123.307 | −61.764 | 1.00 | 47.03 | A |
| ATOM | 2058 | OE2 | GLU | A | 405 | −26.521 | 121.858 | −61.261 | 1.00 | 50.16 | A |
| ATOM | 2059 | C | GLU | A | 405 | −24.292 | 121.563 | −66.726 | 1.00 | 46.65 | A |
| ATOM | 2060 | O | GLU | A | 405 | −24.001 | 122.724 | −67.019 | 1.00 | 47.70 | A |
| ATOM | 2061 | N | HIS | A | 406 | −23.779 | 120.510 | −67.353 | 1.00 | 46.55 | A |
| ATOM | 2062 | CA | HIS | A | 406 | −22.834 | 120.687 | −68.448 | 1.00 | 46.72 | A |
| ATOM | 2063 | CB | HIS | A | 406 | −22.058 | 119.398 | −68.737 | 1.00 | 44.99 | A |
| ATOM | 2064 | CG | HIS | A | 406 | −21.113 | 119.517 | −69.895 | 1.00 | 42.90 | A |
| ATOM | 2065 | CD2 | HIS | A | 406 | −19.763 | 119.607 | −69.940 | 1.00 | 41.46 | A |
| ATOM | 2066 | ND1 | HIS | A | 406 | −21.543 | 119.590 | −71.203 | 1.00 | 41.37 | A |
| ATOM | 2067 | CE1 | HIS | A | 406 | −20.500 | 119.720 | −72.002 | 1.00 | 41.87 | A |
| ATOM | 2068 | NE2 | HIS | A | 406 | −19.408 | 119.733 | −71.261 | 1.00 | 42.52 | A |
| ATOM | 2069 | C | HIS | A | 406 | −23.583 | 121.111 | −69.695 | 1.00 | 46.62 | A |
| ATOM | 2070 | O | HIS | A | 406 | −24.686 | 120.636 | −69.949 | 1.00 | 46.24 | A |
| ATOM | 2071 | N | ARG | A | 407 | −22.967 | 122.006 | −70.463 | 1.00 | 47.11 | A |
| ATOM | 2072 | CA | ARG | A | 407 | −23.545 | 122.528 | −71.697 | 1.00 | 47.66 | A |
| ATOM | 2073 | CB | ARG | A | 407 | −22.448 | 123.141 | −72.580 | 1.00 | 49.51 | A |
| ATOM | 2074 | CG | ARG | A | 407 | −21.572 | 124.152 | −71.875 | 1.00 | 52.53 | A |
| ATOM | 2075 | CD | ARG | A | 407 | −22.414 | 125.173 | −71.129 | 1.00 | 53.78 | A |
| ATOM | 2076 | NE | ARG | A | 407 | −21.612 | 126.235 | −70.527 | 1.00 | 55.27 | A |
| ATOM | 2077 | CZ | ARG | A | 407 | −20.951 | 127.167 | −71.217 | 1.00 | 55.91 | A |
| ATOM | 2078 | NH1 | ARG | A | 407 | −20.980 | 127.175 | −72.551 | 1.00 | 55.67 | A |
| ATOM | 2079 | NH2 | ARG | A | 407 | −20.283 | 128.118 | −70.568 | 1.00 | 56.67 | A |
| ATOM | 2080 | C | ARG | A | 407 | −24.275 | 121.459 | −72.492 | 1.00 | 45.82 | A |
| ATOM | 2081 | O | ARG | A | 407 | −25.354 | 121.696 | −73.032 | 1.00 | 44.38 | A |
| ATOM | 2082 | N | PHE | A | 408 | −23.673 | 120.281 | −72.569 | 1.00 | 44.29 | A |
| ATOM | 2083 | CA | PHE | A | 408 | −24.280 | 119.203 | −73.315 | 1.00 | 42.94 | A |
| ATOM | 2084 | CB | PHE | A | 408 | −23.402 | 117.949 | −73.288 | 1.00 | 41.05 | A |
| ATOM | 2085 | CG | PHE | A | 408 | −23.975 | 116.812 | −74.078 | 1.00 | 39.45 | A |
| ATOM | 2086 | CD1 | PHE | A | 408 | −23.995 | 116.858 | −75.461 | 1.00 | 37.39 | A |
| ATOM | 2087 | CD2 | PHE | A | 408 | −24.567 | 115.725 | −73.432 | 1.00 | 38.00 | A |
| ATOM | 2088 | CE1 | PHE | A | 408 | −24.600 | 115.842 | −76.189 | 1.00 | 36.63 | A |
| ATOM | 2089 | CE2 | PHE | A | 408 | −25.174 | 114.705 | −74.153 | 1.00 | 36.63 | A |
| ATOM | 2090 | CZ | PHE | A | 408 | −25.191 | 114.763 | −75.536 | 1.00 | 35.56 | A |
| ATOM | 2091 | C | PHE | A | 408 | −25.636 | 118.899 | −72.723 | 1.00 | 42.17 | A |
| ATOM | 2092 | O | PHE | A | 408 | −26.639 | 118.901 | −73.418 | 1.00 | 43.14 | A |
| ATOM | 2093 | N | PHE | A | 409 | −25.665 | 118.647 | −71.428 | 1.00 | 41.88 | A |
| ATOM | 2094 | CA | PHE | A | 409 | −26.913 | 118.345 | −70.761 | 1.00 | 41.98 | A |
| ATOM | 2095 | CB | PHE | A | 409 | −26.622 | 117.576 | −69.481 | 1.00 | 39.76 | A |
| ATOM | 2096 | CG | PHE | A | 409 | −26.320 | 116.133 | −69.714 | 1.00 | 37.85 | A |
| ATOM | 2097 | CD1 | PHE | A | 409 | −27.350 | 115.243 | −70.033 | 1.00 | 36.86 | A |
| ATOM | 2098 | CD2 | PHE | A | 409 | −25.015 | 115.664 | −69.644 | 1.00 | 35.28 | A |
| ATOM | 2099 | CE1 | PHE | A | 409 | −27.090 | 113.911 | −70.279 | 1.00 | 35.98 | A |
| ATOM | 2100 | CE2 | PHE | A | 409 | −24.735 | 114.334 | −69.888 | 1.00 | 35.27 | A |
| ATOM | 2101 | CZ | PHE | A | 409 | −25.776 | 113.448 | −70.208 | 1.00 | 35.85 | A |
| ATOM | 2102 | C | PHE | A | 409 | −27.770 | 119.579 | −70.475 | 1.00 | 42.86 | A |
| ATOM | 2103 | O | PHE | A | 409 | −28.807 | 119.478 | −69.837 | 1.00 | 43.83 | A |
| ATOM | 2104 | N | LEU | A | 410 | −27.330 | 120.733 | −70.962 | 1.00 | 43.73 | A |
| ATOM | 2105 | CA | LEU | A | 410 | −28.047 | 121.994 | −70.801 | 1.00 | 44.49 | A |
| ATOM | 2106 | CB | LEU | A | 410 | −27.252 | 123.123 | −71.496 | 1.00 | 45.19 | A |
| ATOM | 2107 | CG | LEU | A | 410 | −26.668 | 124.403 | −70.845 | 1.00 | 45.14 | A |
| ATOM | 2108 | CD1 | LEU | A | 410 | −27.740 | 125.483 | −70.722 | 1.00 | 43.41 | A |
| ATOM | 2109 | CD2 | LEU | A | 410 | −26.024 | 124.074 | −69.499 | 1.00 | 43.54 | A |
| ATOM | 2110 | C | LEU | A | 410 | −29.440 | 121.853 | −71.450 | 1.00 | 44.57 | A |
| ATOM | 2111 | O | LEU | A | 410 | −30.097 | 122.847 | −71.756 | 1.00 | 45.04 | A |
| ATOM | 2112 | N | SER | A | 411 | −29.878 | 120.613 | −71.651 | 1.00 | 42.73 | A |
| ATOM | 2113 | CA | SER | A | 411 | −31.168 | 120.315 | −72.269 | 1.00 | 41.24 | A |
| ATOM | 2114 | CB | SER | A | 411 | −30.933 | 119.605 | −73.610 | 1.00 | 41.27 | A |
| ATOM | 2115 | OG | SER | A | 411 | −31.886 | 118.564 | −73.836 | 1.00 | 36.52 | A |
| ATOM | 2116 | C | SER | A | 411 | −32.125 | 119.458 | −71.432 | 1.00 | 41.48 | A |
| ATOM | 2117 | O | SER | A | 411 | −33.326 | 119.435 | −71.697 | 1.00 | 41.51 | A |
| ATOM | 2118 | N | ILE | A | 412 | −31.610 | 118.747 | −70.431 | 1.00 | 39.95 | A |
| ATOM | 2119 | CA | ILE | A | 412 | −32.470 | 117.874 | −69.636 | 1.00 | 37.71 | A |
| ATOM | 2120 | CB | ILE | A | 412 | −31.795 | 116.483 | −69.412 | 1.00 | 37.46 | A |
| ATOM | 2121 | CG2 | ILE | A | 412 | −32.770 | 115.531 | −68.725 | 1.00 | 36.37 | A |
| ATOM | 2122 | CG1 | ILE | A | 412 | −31.325 | 115.893 | −70.751 | 1.00 | 35.25 | A |
| ATOM | 2123 | CD1 | ILE | A | 412 | −32.228 | 114.820 | −71.358 | 1.00 | 31.16 | A |
| ATOM | 2124 | C | ILE | A | 412 | −32.929 | 118.405 | −68.277 | 1.00 | 36.84 | A |
| ATOM | 2125 | O | ILE | A | 412 | −32.188 | 119.093 | −67.562 | 1.00 | 34.95 | A |
| ATOM | 2126 | N | ASN | A | 413 | −34.176 | 118.068 | −67.953 | 1.00 | 37.15 | A |
| ATOM | 2127 | CA | ASN | A | 413 | −34.822 | 118.417 | −66.695 | 1.00 | 38.79 | A |
| ATOM | 2128 | CB | ASN | A | 413 | −36.339 | 118.546 | −66.895 | 1.00 | 39.70 | A |
| ATOM | 2129 | CG | ASN | A | 413 | −37.090 | 118.765 | −65.589 | 1.00 | 41.29 | A |
| ATOM | 2130 | OD1 | ASN | A | 413 | −36.704 | 119.602 | −64.772 | 1.00 | 45.08 | A |
| ATOM | 2131 | ND2 | ASN | A | 413 | −38.168 | 118.021 | −65.390 | 1.00 | 34.66 | A |
| ATOM | 2132 | C | ASN | A | 413 | −34.506 | 117.218 | −65.817 | 1.00 | 39.23 | A |
| ATOM | 2133 | O | ASN | A | 413 | −35.165 | 116.185 | −65.898 | 1.00 | 38.70 | A |
| ATOM | 2134 | N | TRP | A | 414 | −33.482 | 117.365 | −64.986 | 1.00 | 40.55 | A |
| ATOM | 2135 | CA | TRP | A | 414 | −33.016 | 116.295 | −64.116 | 1.00 | 41.65 | A |
| ATOM | 2136 | CB | TRP | A | 414 | −31.706 | 116.715 | −63.454 | 1.00 | 43.22 | A |
| ATOM | 2137 | CG | TRP | A | 414 | −30.563 | 116.487 | −64.384 | 1.00 | 48.09 | A |
| ATOM | 2138 | CD2 | TRP | A | 414 | −30.184 | 115.233 | −64.973 | 1.00 | 49.22 | A |
| ATOM | 2139 | CE2 | TRP | A | 414 | −29.105 | 115.492 | −65.845 | 1.00 | 49.82 | A |
| ATOM | 2140 | CE3 | TRP | A | 414 | −30.658 | 113.916 | −64.850 | 1.00 | 48.90 | A |
| ATOM | 2141 | CD1 | TRP | A | 414 | −29.722 | 117.424 | −64.904 | 1.00 | 49.09 | A |
| ATOM | 2142 | NE1 | TRP | A | 414 | −28.843 | 116.833 | −65.786 | 1.00 | 50.48 | A |
| ATOM | 2143 | CZ2 | TRP | A | 414 | −28.489 | 114.482 | −66.593 | 1.00 | 50.23 | A |
| ATOM | 2144 | CZ3 | TRP | A | 414 | −30.051 | 112.913 | −65.693 | 1.00 | 48.84 | A |
| ATOM | 2145 | CH2 | TRP | A | 414 | −28.975 | 113.203 | −66.455 | 1.00 | 50.52 | A |
| ATOM | 2146 | C | TRP | A | 414 | −33.972 | 115.759 | −63.083 | 1.00 | 41.45 | A |
| ATOM | 2147 | O | TRP | A | 414 | −33.559 | 115.107 | −62.129 | 1.00 | 42.26 | A |
| ATOM | 2148 | N | GLN | A | 415 | −35.256 | 115.996 | −63.296 | 1.00 | 40.99 | A |
| ATOM | 2149 | CA | GLN | A | 415 | −36.266 | 115.538 | −62.367 | 1.00 | 40.33 | A |
| ATOM | 2150 | CB | GLN | A | 415 | −36.904 | 116.746 | −61.672 | 1.00 | 38.84 | A |
| ATOM | 2151 | CG | GLN | A | 415 | −38.182 | 116.447 | −60.900 | 1.00 | 35.87 | A |
| ATOM | 2152 | CD | GLN | A | 415 | −38.694 | 117.651 | −60.136 | 1.00 | 33.90 | A |
| ATOM | 2153 | OE1 | GLN | A | 415 | −39.843 | 117.687 | −59.726 | 1.00 | 31.97 | A |
| ATOM | 2154 | NE2 | GLN | A | 415 | −37.832 | 118.636 | −59.930 | 1.00 | 31.91 | A |
| ATOM | 2155 | C | GLN | A | 415 | −37.309 | 114.707 | −63.087 | 1.00 | 40.59 | A |
| ATOM | 2156 | O | GLN | A | 415 | −38.042 | 113.967 | −62.459 | 1.00 | 40.18 | A |
| ATOM | 2157 | N | ASP | A | 416 | −37.382 | 114.840 | −64.408 | 1.00 | 41.53 | A |
| ATOM | 2158 | CA | ASP | A | 416 | −38.335 | 114.064 | −65.197 | 1.00 | 43.23 | A |
| ATOM | 2159 | CB | ASP | A | 416 | −38.539 | 114.661 | −66.595 | 1.00 | 44.45 | A |
| ATOM | 2160 | CG | ASP | A | 416 | −39.248 | 116.003 | −66.568 | 1.00 | 44.39 | A |
| ATOM | 2161 | OD1 | ASP | A | 416 | −40.321 | 116.080 | −65.939 | 1.00 | 45.82 | A |
| ATOM | 2162 | OD2 | ASP | A | 416 | −38.737 | 116.970 | −67.172 | 1.00 | 41.69 | A |
| ATOM | 2163 | C | ASP | A | 416 | −37.679 | 112.720 | −65.345 | 1.00 | 43.96 | A |
| ATOM | 2164 | O | ASP | A | 416 | −38.322 | 111.682 | −65.251 | 1.00 | 44.10 | A |
| ATOM | 2165 | N | VAL | A | 417 | −36.374 | 112.775 | −65.579 | 1.00 | 45.55 | A |
| ATOM | 2166 | CA | VAL | A | 417 | −35.536 | 111.599 | −65.751 | 1.00 | 45.69 | A |
| ATOM | 2167 | CB | VAL | A | 417 | −34.050 | 111.954 | −65.583 | 1.00 | 45.81 | A |
| ATOM | 2168 | CG1 | VAL | A | 417 | −33.196 | 110.740 | −65.882 | 1.00 | 45.88 | A |
| ATOM | 2169 | CG2 | VAL | A | 417 | −33.678 | 113.116 | −66.491 | 1.00 | 46.39 | A |
| ATOM | 2170 | C | VAL | A | 417 | −35.881 | 110.565 | −64.707 | 1.00 | 45.58 | A |
| ATOM | 2171 | O | VAL | A | 417 | −36.372 | 109.480 | −65.018 | 1.00 | 46.46 | A |
| ATOM | 2172 | N | VAL | A | 418 | −35.626 | 110.926 | −63.458 | 1.00 | 44.65 | A |
| ATOM | 2173 | CA | VAL | A | 418 | −35.876 | 110.055 | −62.328 | 1.00 | 45.15 | A |
| ATOM | 2174 | CB | VAL | A | 418 | −35.600 | 110.812 | −61.018 | 1.00 | 45.58 | A |
| ATOM | 2175 | CG1 | VAL | A | 418 | −35.572 | 109.864 | −59.863 | 1.00 | 44.96 | A |
| ATOM | 2176 | CG2 | VAL | A | 418 | −34.263 | 111.530 | −61.118 | 1.00 | 46.99 | A |
| ATOM | 2177 | C | VAL | A | 418 | −37.289 | 109.485 | −62.321 | 1.00 | 45.12 | A |
| ATOM | 2178 | O | VAL | A | 418 | −37.522 | 108.419 | −61.762 | 1.00 | 44.65 | A |
| ATOM | 2179 | N | GLN | A | 419 | −38.226 | 110.182 | −62.956 | 1.00 | 46.35 | A |
| ATOM | 2180 | CA | GLN | A | 419 | −39.617 | 109.728 | −63.011 | 1.00 | 47.06 | A |
| ATOM | 2181 | CB | GLN | A | 419 | −40.553 | 110.925 | −62.835 | 1.00 | 48.45 | A |
| ATOM | 2182 | CG | GLN | A | 419 | −42.015 | 110.669 | −63.216 | 1.00 | 52.74 | A |
| ATOM | 2183 | CD | GLN | A | 419 | −42.708 | 109.632 | −62.355 | 1.00 | 54.02 | A |
| ATOM | 2184 | OE1 | GLN | A | 419 | −42.256 | 108.489 | −62.248 | 1.00 | 54.06 | A |
| ATOM | 2185 | NE2 | GLN | A | 419 | −43.823 | 110.024 | −61.743 | 1.00 | 53.61 | A |
| ATOM | 2186 | C | GLN | A | 419 | −39.981 | 108.959 | −64.289 | 1.00 | 46.21 | A |
| ATOM | 2187 | O | GLN | A | 419 | −41.106 | 108.470 | −64.434 | 1.00 | 44.30 | A |
| ATOM | 2188 | N | LYS | A | 420 | −39.018 | 108.818 | −65.196 | 1.00 | 46.01 | A |
| ATOM | 2189 | CA | LYS | A | 420 | −39.259 | 108.128 | −66.457 | 1.00 | 44.73 | A |
| ATOM | 2190 | CB | LYS | A | 420 | −39.751 | 106.700 | −66.205 | 1.00 | 43.74 | A |
| ATOM | 2191 | CG | LYS | A | 420 | −38.701 | 105.746 | −65.664 | 1.00 | 40.89 | A |
| ATOM | 2192 | CD | LYS | A | 420 | −39.279 | 104.355 | −65.407 | 1.00 | 37.22 | A |
| ATOM | 2193 | CE | LYS | A | 420 | −38.152 | 103.355 | −65.173 | 1.00 | 40.17 | A |
| ATOM | 2194 | NZ | LYS | A | 420 | −38.566 | 101.984 | −64.759 | 1.00 | 36.48 | A |
| ATOM | 2195 | C | LYS | A | 420 | −40.316 | 108.916 | −67.226 | 1.00 | 44.79 | A |
| ATOM | 2196 | O | LYS | A | 420 | −41.038 | 108.364 | −68.058 | 1.00 | 43.94 | A |
| ATOM | 2197 | N | LYS | A | 421 | −40.400 | 110.211 | −66.915 | 1.00 | 45.23 | A |
| ATOM | 2198 | CA | LYS | A | 421 | −41.337 | 111.137 | −67.549 | 1.00 | 46.15 | A |
| ATOM | 2199 | CB | LYS | A | 421 | −41.333 | 112.495 | −66.836 | 1.00 | 44.83 | A |
| ATOM | 2200 | CG | LYS | A | 421 | −42.157 | 112.575 | −65.569 | 1.00 | 44.08 | A |
| ATOM | 2201 | CD | LYS | A | 421 | −42.246 | 114.006 | −65.045 | 1.00 | 42.19 | A |
| ATOM | 2202 | CE | LYS | A | 421 | −42.888 | 114.928 | −66.075 | 1.00 | 43.13 | A |
| ATOM | 2203 | NZ | LYS | A | 421 | −43.266 | 116.261 | −65.535 | 1.00 | 39.02 | A |
| ATOM | 2204 | C | LYS | A | 421 | −40.898 | 111.359 | −68.983 | 1.00 | 47.72 | A |
| ATOM | 2205 | O | LYS | A | 421 | −41.692 | 111.727 | −69.847 | 1.00 | 47.61 | A |
| ATOM | 2206 | N | LEU | A | 422 | −39.609 | 111.154 | −69.214 | 1.00 | 48.81 | A |
| ATOM | 2207 | CA | LEU | A | 422 | −39.038 | 111.327 | −70.529 | 1.00 | 50.06 | A |
| ATOM | 2208 | CB | LEU | A | 422 | −37.517 | 111.262 | −70.433 | 1.00 | 50.15 | A |
| ATOM | 2209 | CG | LEU | A | 422 | −36.915 | 112.248 | −69.432 | 1.00 | 50.34 | A |
| ATOM | 2210 | CD1 | LEU | A | 422 | −35.404 | 112.051 | −69.336 | 1.00 | 51.35 | A |
| ATOM | 2211 | CD2 | LEU | A | 422 | −37.240 | 113.660 | −69.864 | 1.00 | 49.35 | A |
| ATOM | 2212 | C | LEU | A | 422 | −39.565 | 110.230 | −71.441 | 1.00 | 50.97 | A |
| ATOM | 2213 | O | LEU | A | 422 | −39.895 | 109.139 | −70.987 | 1.00 | 50.98 | A |
| ATOM | 2214 | N | LEU | A | 423 | −39.661 | 110.528 | −72.729 | 1.00 | 52.60 | A |
| ATOM | 2215 | CA | LEU | A | 423 | −40.145 | 109.553 | −73.686 | 1.00 | 54.55 | A |
| ATOM | 2216 | CB | LEU | A | 423 | −41.081 | 110.226 | −74.697 | 1.00 | 53.81 | A |
| ATOM | 2217 | CG | LEU | A | 423 | −41.938 | 111.411 | −74.210 | 0.00 | 52.10 | A |
| ATOM | 2218 | CD1 | LEU | A | 423 | −42.644 | 112.026 | −75.416 | 1.00 | 51.16 | A |
| ATOM | 2219 | CD2 | LEU | A | 423 | −42.955 | 110.983 | −73.136 | 1.00 | 50.78 | A |
| ATOM | 2220 | C | LEU | A | 423 | −38.912 | 108.987 | −74.393 | 1.00 | 56.58 | A |
| ATOM | 2221 | O | LEU | A | 423 | −38.191 | 109.712 | −75.082 | 1.00 | 56.59 | A |
| ATOM | 2222 | N | PRO | A | 424 | −38.650 | 107.682 | −74.212 | 1.00 | 56.63 | A |
| ATOM | 2223 | CD | PRO | A | 424 | −39.513 | 106.735 | −73.485 | 1.00 | 56.01 | A |
| ATOM | 2224 | CA | PRO | A | 424 | −37.513 | 106.983 | −74.812 | 1.00 | 58.90 | A |
| ATOM | 2225 | CB | PRO | A | 424 | −37.642 | 105.576 | −74.239 | 1.00 | 58.09 | A |
| ATOM | 2226 | CG | PRO | A | 424 | −39.110 | 105.411 | −74.070 | 1.00 | 56.04 | A |
| ATOM | 2227 | C | PRO | A | 424 | −37.530 | 106.997 | −76.341 | 1.00 | 59.64 | A |
| ATOM | 2228 | O | PRO | A | 424 | −38.451 | 106.479 | −76.972 | 1.00 | 58.94 | A |
| ATOM | 2229 | N | PRO | A | 425 | −36.498 | 107.588 | −76.956 | 1.00 | 59.31 | A |
| ATOM | 2230 | CD | PRO | A | 425 | −35.304 | 108.166 | −76.316 | 1.00 | 60.68 | A |
| ATOM | 2231 | CA | PRO | A | 425 | −36.397 | 107.671 | −78.413 | 1.00 | 59.90 | A |
| ATOM | 2232 | CB | PRO | A | 425 | −35.212 | 108.602 | −78.617 | 1.00 | 59.98 | A |
| ATOM | 2233 | CG | PRO | A | 425 | −34.329 | 108.239 | −77.470 | 1.00 | 61.61 | A |
| ATOM | 2234 | C | PRO | A | 425 | −36.187 | 106.312 | −79.061 | 1.00 | 59.43 | A |
| ATOM | 2235 | O | PRO | A | 425 | −35.296 | 106.137 | −79.886 | 1.00 | 60.86 | A |
| ATOM | 2236 | N | PHE | A | 426 | −37.018 | 105.356 | −78.679 | 1.00 | 56.79 | A |
| ATOM | 2237 | CA | PHE | A | 426 | −36.948 | 104.012 | −79.209 | 1.00 | 56.12 | A |
| ATOM | 2238 | CB | PHE | A | 426 | −35.493 | 103.551 | −79.343 | 1.00 | 55.18 | A |
| ATOM | 2239 | CG | PHE | A | 426 | −35.347 | 102.114 | −79.752 | 1.00 | 55.15 | A |
| ATOM | 2240 | CD1 | PHE | A | 426 | −35.822 | 101.672 | −80.976 | 1.00 | 55.09 | A |
| ATOM | 2241 | CD2 | PHE | A | 426 | −34.763 | 101.190 | −78.890 | 1.00 | 56.47 | A |
| ATOM | 2242 | CE1 | PHE | A | 426 | −35.724 | 100.337 | −81.331 | 1.00 | 56.49 | A |
| ATOM | 2243 | CE2 | PHE | A | 426 | −34.660 | 99.841 | −79.239 | 1.00 | 56.52 | A |
| ATOM | 2244 | CZ | PHE | A | 426 | −35.141 | 99.417 | −80.459 | 1.00 | 57.05 | A |
| ATOM | 2245 | C | PHE | A | 426 | −37.683 | 103.121 | −78.233 | 1.00 | 57.34 | A |
| ATOM | 2246 | O | PHE | A | 426 | −37.504 | 103.240 | −77.022 | 1.00 | 57.59 | A |
| ATOM | 2247 | N | LYS | A | 427 | −38.524 | 102.240 | −78.756 | 1.00 | 57.52 | A |
| ATOM | 2248 | CA | LYS | A | 427 | −39.274 | 101.335 | −77.911 | 1.00 | 58.08 | A |
| ATOM | 2249 | CB | LYS | A | 427 | −40.709 | 101.840 | −77.724 | 1.00 | 58.05 | A |
| ATOM | 2250 | CG | LYS | A | 427 | −40.887 | 102.717 | −76.488 | 1.00 | 58.16 | A |
| ATOM | 2251 | CD | LYS | A | 427 | −41.882 | 103.837 | −76.718 | 1.00 | 59.46 | A |
| ATOM | 2252 | CE | LYS | A | 427 | −41.451 | 104.719 | −77.893 | 1.00 | 58.92 | A |
| ATOM | 2253 | NZ | LYS | A | 427 | −42.563 | 105.617 | −78.305 | 1.00 | 59.73 | A |
| ATOM | 2254 | C | LYS | A | 427 | −39.278 | 99.922 | −78.452 | 1.00 | 58.72 | A |
| ATOM | 2255 | O | LYS | A | 427 | −39.852 | 99.644 | −79.498 | 1.00 | 58.34 | A |
| ATOM | 2256 | N | PRO | A | 428 | −38.610 | 99.009 | −77.740 | 1.00 | 60.79 | A |
| ATOM | 2257 | CD | PRO | A | 428 | −37.753 | 99.258 | −76.565 | 1.00 | 60.29 | A |
| ATOM | 2258 | CA | PRO | A | 428 | −38.539 | 97.609 | −78.150 | 1.00 | 62.03 | A |
| ATOM | 2259 | CB | PRO | A | 428 | −37.862 | 96.956 | −76.959 | 1.00 | 62.09 | A |
| ATOM | 2260 | CG | PRO | A | 428 | −36.890 | 98.029 | −76.533 | 1.00 | 60.55 | A |
| ATOM | 2261 | C | PRO | A | 428 | −39.936 | 97.071 | −78.427 | 1.00 | 63.87 | A |
| ATOM | 2262 | O | PRO | A | 428 | −40.861 | 97.313 | −77.661 | 1.00 | 64.43 | A |
| ATOM | 2263 | N | GLN | A | 429 | −40.074 | 96.347 | −79.531 | 1.00 | 65.72 | A |
| ATOM | 2264 | CA | GLN | A | 429 | −41.354 | 95.791 | −79.964 | 1.00 | 68.28 | A |
| ATOM | 2265 | CB | GLN | A | 429 | −41.251 | 95.441 | −81.452 | 1.00 | 69.22 | A |
| ATOM | 2266 | CG | GLN | A | 429 | −40.835 | 96.635 | −82.304 | 0.00 | 71.03 | A |
| ATOM | 2267 | CD | GLN | A | 429 | −40.636 | 96.292 | −83.767 | 0.00 | 71.23 | A |
| ATOM | 2268 | OE1 | GLN | A | 429 | −41.590 | 95.928 | −84.471 | 1.00 | 71.28 | A |
| ATOM | 2269 | NE2 | GLN | A | 429 | −39.389 | 96.410 | −84.237 | 1.00 | 70.79 | A |
| ATOM | 2270 | C | GLN | A | 429 | −41.886 | 94.597 | −79.157 | 1.00 | 67.62 | A |
| ATOM | 2271 | O | GLN | A | 429 | −41.126 | 93.865 | −78.528 | 1.00 | 66.77 | A |
| ATOM | 2272 | N | VAL | A | 430 | −43.205 | 94.414 | −79.189 | 0.00 | 68.55 | A |
| ATOM | 2273 | CA | VAL | A | 430 | −43.866 | 93.336 | −78.451 | 0.00 | 69.19 | A |
| ATOM | 2274 | CB | VAL | A | 430 | −44.684 | 93.921 | −77.262 | 0.00 | 68.71 | A |
| ATOM | 2275 | CG1 | VAL | A | 430 | −45.493 | 92.823 | −76.576 | 1.00 | 67.68 | A |
| ATOM | 2276 | CG2 | VAL | A | 430 | −43.746 | 94.605 | −76.265 | 1.00 | 68.75 | A |
| ATOM | 2277 | C | VAL | A | 430 | −44.807 | 92.498 | −79.327 | 0.00 | 68.52 | A |
| ATOM | 2278 | O | VAL | A | 430 | −45.689 | 93.040 | −79.993 | 0.00 | 68.45 | A |
| ATOM | 2279 | N | THR | A | 431 | −44.615 | 91.179 | −79.323 | 0.00 | 69.17 | A |
| ATOM | 2280 | CA | THR | A | 431 | −45.464 | 90.275 | −80.101 | 0.00 | 70.45 | A |
| ATOM | 2281 | CB | THR | A | 431 | −44.661 | 89.502 | −81.194 | 0.00 | 70.12 | A |
| ATOM | 2282 | OG1 | THR | A | 431 | −45.511 | 89.259 | −82.329 | 1.00 | 68.50 | A |
| ATOM | 2283 | CG2 | THR | A | 431 | −44.140 | 88.141 | −80.646 | 1.00 | 69.39 | A |
| ATOM | 2284 | C | THR | A | 431 | −46.101 | 89.259 | −79.157 | 0.00 | 71.64 | A |
| ATOM | 2285 | O | THR | A | 431 | −47.094 | 88.614 | −79.497 | 0.00 | 70.36 | A |
| ATOM | 2286 | N | SER | A | 432 | −45.518 | 89.129 | −77.968 | 0.00 | 72.76 | A |
| ATOM | 2287 | CA | SER | A | 432 | −46.000 | 88.189 | −76.959 | 0.00 | 73.70 | A |
| ATOM | 2288 | CB | SER | A | 432 | −45.953 | 86.760 | −77.503 | 1.00 | 75.55 | A |
| ATOM | 2289 | OG | SER | A | 432 | −46.054 | 85.810 | −76.448 | 1.00 | 76.12 | A |
| ATOM | 2290 | C | SER | A | 432 | −45.160 | 88.243 | −75.691 | 0.00 | 72.92 | A |
| ATOM | 2291 | O | SER | A | 432 | −43.938 | 88.381 | −75.758 | 0.00 | 73.90 | A |
| ATOM | 2292 | N | GLU | A | 433 | −45.810 | 88.134 | −74.535 | 0.00 | 73.20 | A |
| ATOM | 2293 | CA | GLU | A | 433 | −45.075 | 88.129 | −73.276 | 0.00 | 73.44 | A |
| ATOM | 2294 | CB | GLU | A | 433 | −46.005 | 87.883 | −72.085 | 0.00 | 73.69 | A |
| ATOM | 2295 | CG | GLU | A | 433 | −46.768 | 89.101 | −71.588 | 0.00 | 71.40 | A |
| ATOM | 2296 | CD | GLU | A | 433 | −48.201 | 89.140 | −72.078 | 0.00 | 69.94 | A |
| ATOM | 2297 | OE1 | GLU | A | 433 | −49.096 | 89.443 | −71.259 | 1.00 | 67.25 | A |
| ATOM | 2298 | OE2 | GLU | A | 433 | −48.436 | 88.878 | −73.276 | 1.00 | 68.23 | A |
| ATOM | 2299 | C | GLU | A | 433 | −44.105 | 86.966 | −73.396 | 0.00 | 74.79 | A |
| ATOM | 2300 | O | GLU | A | 433 | −44.311 | 86.078 | −74.225 | 0.00 | 74.19 | A |
| ATOM | 2301 | N | VAL | A | 434 | −43.055 | 86.959 | −72.583 | 0.00 | 74.23 | A |
| ATOM | 2302 | CA | VAL | A | 434 | −42.088 | 85.872 | −72.654 | 1.00 | 75.04 | A |
| ATOM | 2303 | CB | VAL | A | 434 | −42.806 | 84.505 | −72.478 | 1.00 | 74.65 | A |
| ATOM | 2304 | CG1 | VAL | A | 434 | −41.824 | 83.354 | −72.690 | 1.00 | 74.90 | A |
| ATOM | 2305 | CG2 | VAL | A | 434 | −43.451 | 84.434 | −71.096 | 1.00 | 73.65 | A |
| ATOM | 2306 | C | VAL | A | 434 | −41.432 | 85.946 | −74.038 | 1.00 | 74.75 | A |
| ATOM | 2307 | O | VAL | A | 434 | −41.941 | 85.382 | −75.011 | 1.00 | 76.78 | A |
| ATOM | 2308 | N | ASP | A | 435 | −40.310 | 86.655 | −74.119 | 0.00 | 73.96 | A |
| ATOM | 2309 | CA | ASP | A | 435 | −39.590 | 86.828 | −75.378 | 1.00 | 72.40 | A |
| ATOM | 2310 | CB | ASP | A | 435 | −40.485 | 87.560 | −76.387 | 1.00 | 72.75 | A |
| ATOM | 2311 | CG | ASP | A | 435 | −39.739 | 87.981 | −77.647 | 1.00 | 71.98 | A |
| ATOM | 2312 | OD1 | ASP | A | 435 | −40.394 | 88.478 | −78.589 | 1.00 | 71.33 | A |
| ATOM | 2313 | OD2 | ASP | A | 435 | −38.504 | 87.821 | −77.700 | 1.00 | 71.98 | A |
| ATOM | 2314 | C | ASP | A | 435 | −38.326 | 87.640 | −75.131 | 1.00 | 72.65 | A |
| ATOM | 2315 | O | ASP | A | 435 | −38.389 | 88.715 | −74.532 | 1.00 | 73.50 | A |
| ATOM | 2316 | N | THR | A | 436 | −37.183 | 87.134 | −75.596 | 1.00 | 71.32 | A |
| ATOM | 2317 | CA | THR | A | 436 | −35.903 | 87.827 | −75.412 | 1.00 | 70.22 | A |
| ATOM | 2318 | CB | THR | A | 436 | −34.970 | 87.038 | −74.452 | 1.00 | 68.79 | A |
| ATOM | 2319 | OG1 | THR | A | 436 | −34.423 | 85.906 | −75.139 | 1.00 | 67.56 | A |
| ATOM | 2320 | CG2 | THR | A | 436 | −35.745 | 86.552 | −73.226 | 1.00 | 66.02 | A |
| ATOM | 2321 | C | THR | A | 436 | −35.204 | 87.980 | −76.763 | 1.00 | 69.55 | A |
| ATOM | 2322 | O | THR | A | 436 | −33.978 | 88.034 | −76.841 | 1.00 | 69.24 | A |
| ATOM | 2323 | N | ARG | A | 437 | −36.025 | 88.061 | −77.808 | 1.00 | 69.77 | A |
| ATOM | 2324 | CA | ARG | A | 437 | −35.631 | 88.177 | −79.218 | 1.00 | 68.79 | A |
| ATOM | 2325 | CB | ARG | A | 437 | −36.911 | 88.401 | −80.046 | 1.00 | 68.01 | A |
| ATOM | 2326 | CG | ARG | A | 437 | −36.828 | 88.059 | −81.534 | 1.00 | 66.69 | A |
| ATOM | 2327 | CD | ARG | A | 437 | −38.218 | 87.811 | −82.127 | 1.00 | 65.27 | A |
| ATOM | 2328 | NE | ARG | A | 437 | −38.843 | 86.610 | −81.541 | 1.00 | 63.62 | A |
| ATOM | 2329 | CZ | ARG | A | 437 | −39.925 | 85.995 | −82.029 | 1.00 | 59.93 | A |
| ATOM | 2330 | NH1 | ARG | A | 437 | −40.534 | 86.456 | −83.122 | 1.00 | 58.41 | A |
| ATOM | 2331 | NH2 | ARG | A | 437 | −40.373 | 84.904 | −81.436 | 1.00 | 56.18 | A |
| ATOM | 2332 | C | ARG | A | 437 | −34.562 | 89.216 | −79.601 | 1.00 | 67.75 | A |
| ATOM | 2333 | O | ARG | A | 437 | −34.003 | 89.142 | −80.696 | 1.00 | 66.59 | A |
| ATOM | 2334 | N | TYR | A | 438 | −34.276 | 90.171 | −78.714 | 1.00 | 68.22 | A |
| ATOM | 2335 | CA | TYR | A | 438 | −33.270 | 91.218 | −78.984 | 1.00 | 67.16 | A |
| ATOM | 2336 | CB | TYR | A | 438 | −33.614 | 92.517 | −78.236 | 1.00 | 65.63 | A |
| ATOM | 2337 | CG | TYR | A | 438 | −34.994 | 93.048 | −78.517 | 1.00 | 64.03 | A |
| ATOM | 2338 | CD1 | TYR | A | 438 | −35.954 | 93.098 | −77.513 | 1.00 | 63.19 | A |
| ATOM | 2339 | CE1 | TYR | A | 438 | −37.246 | 93.527 | −77.772 | 1.00 | 63.50 | A |
| ATOM | 2340 | CD2 | TYR | A | 438 | −35.357 | 93.453 | −79.803 | 1.00 | 64.73 | A |
| ATOM | 2341 | CE2 | TYR | A | 438 | −36.652 | 93.892 | −80.083 | 1.00 | 64.79 | A |
| ATOM | 2342 | CZ | TYR | A | 438 | −37.593 | 93.926 | −79.059 | 1.00 | 64.10 | A |
| ATOM | 2343 | OH | TYR | A | 438 | −38.871 | 94.372 | −79.323 | 1.00 | 64.05 | A |
| ATOM | 2344 | C | TYR | A | 438 | −31.865 | 90.793 | −78.572 | 1.00 | 67.56 | A |
| ATOM | 2345 | O | TYR | A | 438 | −30.917 | 91.567 | −78.664 | 1.00 | 66.70 | A |
| ATOM | 2346 | N | PHE | A | 439 | −31.739 | 89.556 | −78.119 | 1.00 | 68.41 | A |
| ATOM | 2347 | CA | PHE | A | 439 | −30.464 | 89.033 | −77.663 | 1.00 | 69.60 | A |
| ATOM | 2348 | CB | PHE | A | 439 | −30.712 | 88.179 | −76.414 | 1.00 | 68.48 | A |
| ATOM | 2349 | CG | PHE | A | 439 | −31.331 | 88.957 | −75.259 | 1.00 | 67.67 | A |
| ATOM | 2350 | CD1 | PHE | A | 439 | −32.211 | 90.016 | −75.495 | 1.00 | 66.74 | A |
| ATOM | 2351 | CD2 | PHE | A | 439 | −31.043 | 88.621 | −73.939 | 1.00 | 66.90 | A |
| ATOM | 2352 | CE1 | PHE | A | 439 | −32.792 | 90.724 | −74.440 | 1.00 | 66.23 | A |
| ATOM | 2353 | CE2 | PHE | A | 439 | −31.619 | 89.321 | −72.879 | 1.00 | 65.64 | A |
| ATOM | 2354 | CZ | PHE | A | 439 | −32.495 | 90.376 | −73.133 | 1.00 | 65.83 | A |
| ATOM | 2355 | C | PHE | A | 439 | −29.715 | 88.264 | −78.762 | 1.00 | 70.52 | A |
| ATOM | 2356 | O | PHE | A | 439 | −29.234 | 88.880 | −79.720 | 1.00 | 70.33 | A |
| ATOM | 2357 | N | ASP | A | 440 | −29.614 | 86.940 | −78.653 | 1.00 | 71.72 | A |
| ATOM | 2358 | CA | ASP | A | 440 | −28.889 | 86.189 | −79.687 | 1.00 | 73.06 | A |
| ATOM | 2359 | CB | ASP | A | 440 | −27.404 | 86.606 | −79.640 | 1.00 | 71.34 | A |
| ATOM | 2360 | CG | ASP | A | 440 | −26.661 | 86.333 | −80.935 | 1.00 | 68.74 | A |
| ATOM | 2361 | OD1 | ASP | A | 440 | −25.527 | 86.844 | −81.076 | 1.00 | 66.33 | A |
| ATOM | 2362 | OD2 | ASP | A | 440 | −27.200 | 85.614 | −81.800 | 1.00 | 67.42 | A |
| ATOM | 2363 | C | ASP | A | 440 | −29.028 | 84.651 | −79.599 | 1.00 | 74.04 | A |
| ATOM | 2364 | O | ASP | A | 440 | −29.455 | 84.098 | −78.572 | 1.00 | 73.71 | A |
| ATOM | 2365 | N | ASP | A | 441 | −28.668 | 83.981 | −80.697 | 1.00 | 74.91 | A |
| ATOM | 2366 | CA | ASP | A | 441 | −28.726 | 82.522 | −80.798 | 1.00 | 75.49 | A |
| ATOM | 2367 | CB | ASP | A | 441 | −28.896 | 82.091 | −82.257 | 1.00 | 75.66 | A |
| ATOM | 2368 | CG | ASP | A | 441 | −30.107 | 82.719 | −82.922 | 1.00 | 76.58 | A |
| ATOM | 2369 | OD1 | ASP | A | 441 | −30.384 | 82.355 | −84.090 | 1.00 | 76.57 | A |
| ATOM | 2370 | OD2 | ASP | A | 441 | −30.773 | 83.572 | −82.286 | 1.00 | 75.89 | A |
| ATOM | 2371 | C | ASP | A | 441 | −27.444 | 81.891 | −80.266 | 1.00 | 75.57 | A |
| ATOM | 2372 | O | ASP | A | 441 | −26.484 | 81.715 | −81.014 | 1.00 | 75.00 | A |
| ATOM | 2373 | N | ALA | A | 442 | −27.430 | 81.546 | −78.983 | 1.00 | 75.85 | A |
| ATOM | 2374 | CA | ALA | A | 442 | −26.255 | 80.927 | −78.365 | 1.00 | 76.13 | A |
| ATOM | 2375 | CB | ALA | A | 442 | −25.298 | 82.008 | −77.856 | 1.00 | 74.63 | A |
| ATOM | 2376 | C | ALA | A | 442 | −26.643 | 79.975 | −77.224 | 1.00 | 76.57 | A |
| ATOM | 2377 | O | ALA | A | 442 | −27.666 | 79.270 | −77.377 | 1.00 | 76.87 | A |
| ATOM | 2378 | OXT | ALA | A | 442 | −25.920 | 79.929 | −76.203 | 1.00 | 75.20 | A |

TABLE 3
Coordinate data for pΔPH-PKBβ-ΔC (second batch)

REMARK coordinates from minimization refinement
REMARK refinement resolution: 500.0-2.3 A
REMARK starting r = 0.2431 free_r = 0.3043
REMARK final r = 0.2374 free_r = 0.3087
REMARK rmsd bonds = 0.010503 rmsd angles = 1.53593
REMARK wa = 4.5
REMARK target = mlf cycles = 1 steps = 80
REMARK sg = P4 (1) 2 (1) 2 a = 148.405 b = 148.405 c = 38.547 alpha = 90 beta = 90
gamma = 90
REMARK parameter file 1: CNS_TOPPAR: protein_rep.param
REMARK parameter file 2: CNS_TOPPAR: water_rep.param
REMARK molecular structure file: water_pick.mtf
REMARK input coordinates: water_pick.pdb
REMARK reflection file = pkb-33-all-new.fob
REMARK ncs = none
REMARK B-correction resolution: 6.0-2.3
REMARK initial B-factor correction applied to fobs:
REMARK B11 = −8.472 B22 = −8.472 B33 = 16.944
REMARK B12 = 0.000 B13 = 0.000 B23 = 0.000
REMARK B-factor correction applied to coordinate array B: −1.018
REMARK bulk solvent: density level = 0.335739 e/A\hat{ }3, B-factor = 54.1491 A\hat{ }2
REMARK reflections with |Fobs|/sigma_F < 0.0 rejected
REMARK reflections with |Fobs| > 10000 * rms (Fobs) rejected
REMARK theoretical total number of refl. in resol. range: 19801 (100.0%)
REMARK number of unobserved reflections (no entry or |F| =0): 827 (4.2%)
REMARK number of reflections rejected: 0 (0.0%)
REMARK total number of reflections used: 18974 (95.8%)
REMARK number of reflections in working set: 17576 (88.8%)
REMARK number of reflections in test set: 1398 (7.1%)
CRYST1 148.405 148.405 38.547 90.00 90.00 90.00 P 41 21 2
REMARK FILENAME = “minimize.pdb”
REMARK DATE: 23-Nov-01 19:15:46
REMARK VERSION: 1.1

| | | | | | | | | | | | |
|---|---|---|---|---|---|---|---|---|---|---|---|
| ATOM | 1 | CB | ALA | | 146 | −37.642 | 81.084 | −46.450 | 1.00 | 75.24 | |
| ATOM | 2 | C | ALA | | 146 | −38.383 | 81.091 | −48.848 | 1.00 | 74.66 | |
| ATOM | 3 | O | ALA | | 146 | −39.041 | 80.113 | −49.229 | 1.00 | 76.10 | |
| ATOM | 4 | N | ALA | | 146 | −40.033 | 81.409 | −47.060 | 1.00 | 74.47 | |
| ATOM | 5 | CA | ALA | | 146 | −38.626 | 81.691 | −47.470 | 1.00 | 74.54 | |
| ATOM | 6 | N | ALA | | 147 | −37.448 | 81.673 | −49.600 | 1.00 | 72.95 | |
| ATOM | 7 | CA | ALA | | 147 | −37.135 | 81.164 | −50.931 | 1.00 | 69.87 | |
| ATOM | 8 | CB | ALA | | 147 | −36.605 | 82.281 | −51.822 | 1.00 | 69.59 | |
| ATOM | 9 | C | ALA | | 147 | −36.107 | 80.051 | −50.824 | 1.00 | 67.47 | |
| ATOM | 10 | O | ALA | | 147 | −35.286 | 80.029 | −49.909 | 1.00 | 66.70 | |
| ATOM | 11 | N | THR | | 148 | −36.166 | 79.129 | −51.773 | 1.00 | 65.70 | |
| ATOM | 12 | CA | THR | | 148 | −35.249 | 77.999 | −51.824 | 1.00 | 63.28 | |
| ATOM | 13 | CB | THR | | 148 | −35.790 | 76.808 | −51.001 | 1.00 | 63.55 | |
| ATOM | 14 | OG1 | THR | | 148 | −35.170 | 76.818 | −49.709 | 1.00 | 64.84 | |
| ATOM | 15 | CG2 | THR | | 148 | −35.527 | 75.477 | −51.687 | 1.00 | 62.78 | |
| ATOM | 16 | C | THR | | 148 | −35.048 | 77.579 | −53.268 | 1.00 | 62.60 | |
| ATOM | 17 | O | THR | | 148 | −35.898 | 77.841 | −54.128 | 1.00 | 61.96 | |
| ATOM | 18 | N | MET | | 149 | −33.921 | 76.927 | −53.527 | 1.00 | 60.64 | |
| ATOM | 19 | CA | MET | | 149 | −33.597 | 76.474 | −54.865 | 1.00 | 59.94 | |
| ATOM | 20 | CB | MET | | 149 | −32.461 | 75.462 | −54.816 | 1.00 | 59.38 | |
| ATOM | 21 | CG | MET | | 149 | −31.139 | 76.109 | −54.570 | 1.00 | 59.25 | |
| ATOM | 22 | SD | MET | | 149 | −30.742 | 77.184 | −55.938 | 1.00 | 56.44 | |
| ATOM | 23 | CE | MET | | 149 | −28.996 | 76.720 | −56.112 | 1.00 | 58.07 | |
| ATOM | 24 | C | MET | | 149 | −34.762 | 75.864 | −55.594 | 1.00 | 60.31 | |
| ATOM | 25 | O | MET | | 149 | −34.903 | 76.031 | −56.805 | 1.00 | 61.48 | |
| ATOM | 26 | N | ASN | | 150 | −35.606 | 75.156 | −54.857 | 1.00 | 62.05 | |
| ATOM | 27 | CA | ASN | | 150 | −36.745 | 74.498 | −55.470 | 1.00 | 62.17 | |
| ATOM | 28 | CB | ASN | | 150 | −37.275 | 73.404 | −54.549 | 1.00 | 64.49 | |
| ATOM | 29 | CG | ASN | | 150 | −38.268 | 72.507 | −55.245 | 1.00 | 66.43 | |
| ATOM | 30 | OD1 | ASN | | 150 | −39.189 | 71.991 | −54.619 | 1.00 | 69.01 | |
| ATOM | 31 | ND2 | ASN | | 150 | −38.091 | 72.317 | −56.554 | 1.00 | 66.61 | |
| ATOM | 32 | C | ASN | | 150 | −37.868 | 75.444 | −55.873 | 1.00 | 60.81 | |
| ATOM | 33 | O | ASN | | 150 | −38.844 | 75.028 | −56.478 | 1.00 | 61.24 | |
| ATOM | 34 | N | ASP | | 151 | −37.731 | 76.718 | −55.546 | 1.00 | 59.88 | |
| ATOM | 35 | CA | ASP | | 151 | −38.738 | 77.700 | −55.931 | 1.00 | 60.47 | |
| ATOM | 36 | CB | ASP | | 151 | −38.676 | 78.926 | −55.002 | 1.00 | 62.92 | |
| ATOM | 37 | CG | ASP | | 151 | −39.337 | 78.690 | −53.655 | 1.00 | 64.34 | |
| ATOM | 38 | OD1 | ASP | | 151 | −40.502 | 78.241 | −53.661 | 1.00 | 67.09 | |
| ATOM | 39 | OD2 | ASP | | 151 | −38.711 | 78.969 | −52.602 | 1.00 | 63.82 | |
| ATOM | 40 | C | ASP | | 151 | −38.518 | 78.188 | −57.374 | 1.00 | 60.17 | |
| ATOM | 41 | O | ASP | | 151 | −39.284 | 79.009 | −57.884 | 1.00 | 57.73 | |
| ATOM | 42 | N | PHE | | 152 | −37.483 | 77.692 | −58.047 | 1.00 | 60.29 | |
| ATOM | 43 | CA | PHE | | 152 | −37.210 | 78.195 | −59.388 | 1.00 | 59.56 | |
| ATOM | 44 | CB | PHE | | 152 | −36.050 | 79.196 | −59.305 | 1.00 | 56.60 | |
| ATOM | 45 | CG | PHE | | 152 | −36.232 | 80.251 | −58.245 | 1.00 | 53.19 | |
| ATOM | 46 | CD1 | PHE | | 152 | −37.003 | 81.385 | −58.494 | 1.00 | 52.09 | |
| ATOM | 47 | CD2 | PHE | | 152 | −35.653 | 80.097 | −56.984 | 1.00 | 50.65 | |
| ATOM | 48 | CE1 | PHE | | 152 | −37.199 | 82.363 | −57.495 | 1.00 | 48.55 | |
| ATOM | 49 | CE2 | PHE | | 152 | −35.841 | 81.061 | −55.983 | 1.00 | 50.10 | |
| ATOM | 50 | CZ | PHE | | 152 | −36.620 | 82.201 | −56.244 | 1.00 | 47.82 | |
| ATOM | 51 | C | PHE | | 152 | −36.923 | 77.183 | −60.493 | 1.00 | 60.88 | |
| ATOM | 52 | O | PHE | | 152 | −36.413 | 76.086 | −60.246 | 1.00 | 62.22 | |
| ATOM | 53 | N | ASP | | 153 | −37.268 | 77.572 | −61.721 | 1.00 | 62.21 | |
| ATOM | 54 | CA | ASP | | 153 | −37.032 | 76.753 | −62.904 | 1.00 | 62.60 | |
| ATOM | 55 | CB | ASP | | 153 | −38.188 | 76.860 | −63.902 | 1.00 | 66.09 | |
| ATOM | 56 | CG | ASP | | 153 | −39.500 | 76.340 | −63.349 | 1.00 | 69.91 | |
| ATOM | 57 | OD1 | ASP | | 153 | −39.570 | 75.140 | −62.990 | 1.00 | 71.36 | |
| ATOM | 58 | OD2 | ASP | | 153 | −40.468 | 77.135 | −63.284 | 1.00 | 72.40 | |
| ATOM | 59 | C | ASP | | 153 | −35.798 | 77.320 | −63.574 | 1.00 | 61.47 | |
| ATOM | 60 | O | ASP | | 153 | −35.665 | 78.536 | −63.706 | 1.00 | 60.46 | |
| ATOM | 61 | N | TYR | | 154 | −34.897 | 76.447 | −63.997 | 1.00 | 60.59 | |
| ATOM | 62 | CA | TYR | | 154 | −33.692 | 76.894 | −64.676 | 1.00 | 59.06 | |
| ATOM | 63 | CB | TYR | | 154 | −32.619 | 75.826 | −64.608 | 1.00 | 58.21 | |
| ATOM | 64 | CG | TYR | | 154 | −31.473 | 76.086 | −65.557 | 1.00 | 58.30 | |
| ATOM | 65 | CD1 | TYR | | 154 | −30.431 | 76.936 | −65.209 | 1.00 | 57.17 | |
| ATOM | 66 | CE1 | TYR | | 154 | −29.362 | 77.135 | −66.064 | 1.00 | 57.83 | |
| ATOM | 67 | CD2 | TYR | | 154 | −31.422 | 75.453 | −66.794 | 1.00 | 57.54 | |
| ATOM | 68 | CE2 | TYR | | 154 | −30.367 | 75.640 | −67.652 | 1.00 | 58.09 | |
| ATOM | 69 | CZ | TYR | | 154 | −29.332 | 76.475 | −67.287 | 1.00 | 59.85 | |
| ATOM | 70 | OH | TYR | | 154 | −28.245 | 76.592 | −68.129 | 1.00 | 61.95 | |
| ATOM | 71 | C | TYR | | 154 | −33.948 | 77.204 | −66.145 | 1.00 | 58.79 | |
| ATOM | 72 | O | TYR | | 154 | −34.448 | 76.354 | −66.878 | 1.00 | 60.40 | |
| ATOM | 73 | N | LEU | | 155 | −33.588 | 78.410 | −66.581 | 1.00 | 58.11 | |
| ATOM | 74 | CA | LEU | | 155 | −33.763 | 78.784 | −67.980 | 1.00 | 55.79 | |
| ATOM | 75 | CB | LEU | | 155 | −34.390 | 80.163 | −68.073 | 1.00 | 55.60 | |
| ATOM | 76 | CG | LEU | | 155 | −35.886 | 79.883 | −67.996 | 1.00 | 56.32 | |
| ATOM | 77 | CD1 | LEU | | 155 | −36.631 | 81.037 | −67.446 | 1.00 | 54.90 | |
| ATOM | 78 | CD2 | LEU | | 155 | −36.381 | 79.522 | −69.392 | 1.00 | 57.99 | |
| ATOM | 79 | C | LEU | | 155 | −32.456 | 78.723 | −68.736 | 1.00 | 53.65 | |
| ATOM | 80 | O | LEU | | 155 | −32.336 | 77.986 | −69.701 | 1.00 | 52.78 | |
| ATOM | 81 | N | LYS | | 156 | −31.463 | 79.482 | −68.301 | 1.00 | 53.65 | |
| ATOM | 82 | CA | LYS | | 156 | −30.170 | 79.435 | −68.972 | 1.00 | 54.02 | |
| ATOM | 83 | CB | LYS | | 156 | −30.243 | 80.086 | −70.357 | 1.00 | 55.68 | |
| ATOM | 84 | CG | LYS | | 156 | −30.412 | 81.588 | −70.335 | 1.00 | 58.53 | |
| ATOM | 85 | CD | LYS | | 156 | −30.300 | 82.164 | −71.738 | 1.00 | 60.28 | |
| ATOM | 86 | CE | LYS | | 156 | −29.978 | 83.651 | −71.688 | 1.00 | 60.98 | |
| ATOM | 87 | NZ | LYS | | 156 | −29.678 | 84.179 | −73.055 | 1.00 | 63.58 | |
| ATOM | 88 | C | LYS | | 156 | −29.094 | 80.119 | −68.164 | 1.00 | 53.71 | |
| ATOM | 89 | O | LYS | | 156 | −29.366 | 80.790 | −67.165 | 1.00 | 52.50 | |
| ATOM | 90 | N | LEU | | 157 | −27.860 | 79.948 | −68.605 | 1.00 | 54.56 | |
| ATOM | 91 | CA | LEU | | 157 | −26.746 | 80.571 | −67.921 | 1.00 | 56.09 | |
| ATOM | 92 | CB | LEU | | 157 | −25.455 | 79.797 | −68.185 | 1.00 | 56.31 | |
| ATOM | 93 | CG | LEU | | 157 | −24.186 | 80.379 | −67.559 | 1.00 | 55.75 | |
| ATOM | 94 | CD1 | LEU | | 157 | −24.387 | 80.695 | −66.079 | 1.00 | 54.49 | |
| ATOM | 95 | CD2 | LEU | | 157 | −23.077 | 79.379 | −67.753 | 1.00 | 58.24 | |
| ATOM | 96 | C | LEU | | 157 | −26.574 | 82.002 | −68.400 | 1.00 | 57.27 | |
| ATOM | 97 | O | LEU | | 157 | −26.436 | 82.246 | −69.592 | 1.00 | 57.09 | |
| ATOM | 98 | N | LEU | | 158 | −26.588 | 82.949 | −67.472 | 1.00 | 58.72 | |
| ATOM | 99 | CA | LEU | | 158 | −26.406 | 84.339 | −67.847 | 1.00 | 61.37 | |
| ATOM | 100 | CB | LEU | | 158 | −27.269 | 85.246 | −66.975 | 1.00 | 59.04 | |
| ATOM | 101 | CG | LEU | | 158 | −28.732 | 85.225 | −67.410 | 1.00 | 56.77 | |
| ATOM | 102 | CD1 | LEU | | 158 | −29.581 | 86.052 | −66.461 | 1.00 | 57.48 | |
| ATOM | 103 | CD2 | LEU | | 158 | −28.829 | 85.772 | −68.832 | 1.00 | 56.10 | |
| ATOM | 104 | C | LEU | | 158 | −24.942 | 84.697 | −67.713 | 1.00 | 63.74 | |
| ATOM | 105 | O | LEU | | 158 | −24.428 | 85.520 | −68.458 | 1.00 | 63.88 | |
| ATOM | 106 | N | GLY | | 159 | −24.264 | 84.056 | −66.772 | 1.00 | 67.31 | |
| ATOM | 107 | CA | GLY | | 159 | −22.853 | 84.332 | −66.589 | 1.00 | 71.22 | |
| ATOM | 108 | C | GLY | | 159 | −22.125 | 83.436 | −65.602 | 1.00 | 73.75 | |
| ATOM | 109 | O | GLY | | 159 | −22.663 | 83.050 | −64.564 | 1.00 | 73.51 | |
| ATOM | 110 | N | LYS | | 160 | −20.891 | 83.092 | −65.938 | 1.00 | 76.64 | |
| ATOM | 111 | CA | LYS | | 160 | −20.070 | 82.274 | −65.060 | 1.00 | 80.27 | |
| ATOM | 112 | CB | LYS | | 160 | −19.468 | 81.096 | −65.826 | 1.00 | 80.27 | |
| ATOM | 113 | CG | LYS | | 160 | −18.685 | 80.125 | −64.949 | 1.00 | 82.00 | |
| ATOM | 114 | CD | LYS | | 160 | −18.167 | 78.934 | −65.758 | 1.00 | 83.36 | |
| ATOM | 115 | CE | LYS | | 160 | −17.410 | 77.928 | −64.888 | 1.00 | 82.96 | |
| ATOM | 116 | NZ | LYS | | 160 | −16.799 | 76.854 | −65.724 | 1.00 | 81.28 | |
| ATOM | 117 | C | LYS | | 160 | −18.968 | 83.214 | −64.580 | 1.00 | 82.51 | |
| ATOM | 118 | O | LYS | | 160 | −18.846 | 84.334 | −65.081 | 1.00 | 83.54 | |
| ATOM | 119 | N | GLY | | 161 | −18.168 | 82.782 | −63.616 | 1.00 | 84.63 | |
| ATOM | 120 | CA | GLY | | 161 | −17.112 | 83.655 | −63.135 | 1.00 | 86.72 | |
| ATOM | 121 | C | GLY | | 161 | −16.292 | 83.081 | −61.999 | 1.00 | 87.97 | |
| ATOM | 122 | O | GLY | | 161 | −16.583 | 81.992 | −61.489 | 1.00 | 88.29 | |
| ATOM | 123 | N | THR | | 162 | −15.262 | 83.824 | −61.604 | 1.00 | 88.34 | |
| ATOM | 124 | CA | THR | | 162 | −14.376 | 83.406 | −60.525 | 1.00 | 88.46 | |
| ATOM | 125 | CB | THR | | 162 | −13.221 | 84.393 | −60.385 | 1.00 | 89.84 | |
| ATOM | 126 | OG1 | THR | | 162 | −12.916 | 84.938 | −61.678 | 1.00 | 90.96 | |
| ATOM | 127 | CG2 | THR | | 162 | −11.985 | 83.690 | −59.827 | 1.00 | 90.13 | |
| ATOM | 128 | C | THR | | 162 | −15.170 | 83.376 | −59.224 | 1.00 | 87.43 | |
| ATOM | 129 | O | THR | | 162 | −15.015 | 82.476 | −58.397 | 1.00 | 87.84 | |
| ATOM | 130 | N | PHE | | 163 | −16.025 | 84.381 | −59.074 | 1.00 | 85.83 | |
| ATOM | 131 | CA | PHE | | 163 | −16.904 | 84.562 | −57.923 | 1.00 | 84.10 | |
| ATOM | 132 | CB | PHE | | 163 | −17.533 | 85.938 | −58.047 | 1.00 | 84.86 | |
| ATOM | 133 | CG | PHE | | 163 | −18.142 | 86.187 | −59.403 | 1.00 | 85.76 | |
| ATOM | 134 | CD1 | PHE | | 163 | −19.508 | 85.984 | −59.622 | 1.00 | 85.73 | |
| ATOM | 135 | CD2 | PHE | | 163 | −17.342 | 86.594 | −60.475 | 1.00 | 86.08 | |
| ATOM | 136 | CE1 | PHE | | 163 | −20.074 | 86.184 | −60.888 | 1.00 | 86.00 | |
| ATOM | 137 | CE2 | PHE | | 163 | −17.894 | 86.796 | −61.745 | 1.00 | 86.68 | |
| ATOM | 138 | CZ | PHE | | 163 | −19.265 | 86.591 | −61.951 | 1.00 | 86.71 | |
| ATOM | 139 | C | PHE | | 163 | −18.017 | 83.494 | −57.849 | 1.00 | 82.58 | |
| ATOM | 140 | O | PHE | | 163 | −18.457 | 83.100 | −56.764 | 1.00 | 82.36 | |
| ATOM | 141 | N | GLY | | 164 | −18.471 | 83.037 | −59.012 | 1.00 | 79.86 | |
| ATOM | 142 | CA | GLY | | 164 | −19.530 | 82.049 | −59.056 | 1.00 | 76.17 | |
| ATOM | 143 | C | GLY | | 164 | −20.227 | 82.093 | −60.402 | 1.00 | 73.69 | |
| ATOM | 144 | O | GLY | | 164 | −19.574 | 82.109 | −61.448 | 1.00 | 74.26 | |
| ATOM | 145 | N | LYS | | 165 | −21.553 | 82.133 | −60.389 | 1.00 | 69.87 | |
| ATOM | 146 | CA | LYS | | 165 | −22.312 | 82.154 | −61.632 | 1.00 | 65.10 | |
| ATOM | 147 | CB | LYS | | 165 | −22.612 | 80.709 | −62.064 | 1.00 | 66.05 | |
| ATOM | 148 | CG | LYS | | 165 | −23.469 | 79.912 | −61.074 | 1.00 | 66.47 | |
| ATOM | 149 | CD | LYS | | 165 | −23.502 | 78.419 | −61.418 | 1.00 | 68.61 | |
| ATOM | 150 | CE | LYS | | 165 | −22.228 | 77.699 | −60.963 | 1.00 | 69.56 | |
| ATOM | 151 | NZ | LYS | | 165 | −22.080 | 76.323 | −61.531 | 1.00 | 69.28 | |
| ATOM | 152 | C | LYS | | 165 | −23.619 | 82.933 | −61.498 | 1.00 | 62.02 | |
| ATOM | 153 | O | LYS | | 165 | −24.043 | 83.269 | −60.388 | 1.00 | 59.79 | |
| ATOM | 154 | N | VAL | | 166 | −24.248 | 83.224 | −62.636 | 1.00 | 58.85 | |
| ATOM | 155 | CA | VAL | | 166 | −25.520 | 83.936 | −62.661 | 1.00 | 56.27 | |
| ATOM | 156 | CB | VAL | | 166 | −25.372 | 85.413 | −63.112 | 1.00 | 55.61 | |
| ATOM | 157 | CG1 | VAL | | 166 | −26.754 | 86.063 | −63.228 | 1.00 | 53.77 | |
| ATOM | 158 | CG2 | VAL | | 166 | −24.515 | 86.197 | −62.095 | 1.00 | 54.35 | |
| ATOM | 159 | C | VAL | | 166 | −26.407 | 83.192 | −63.637 | 1.00 | 56.59 | |
| ATOM | 160 | O | VAL | | 166 | −26.075 | 83.022 | −64.815 | 1.00 | 54.74 | |
| ATOM | 161 | N | ILE | | 167 | −27.540 | 82.743 | −63.120 | 1.00 | 55.92 | |
| ATOM | 162 | CA | ILE | | 167 | −28.480 | 81.965 | −63.898 | 1.00 | 55.67 | |
| ATOM | 163 | CB | ILE | | 167 | −28.842 | 80.645 | −63.158 | 1.00 | 55.84 | |
| ATOM | 164 | CG2 | ILE | | 167 | −29.868 | 79.876 | −63.947 | 1.00 | 57.49 | |
| ATOM | 165 | CG1 | ILE | | 167 | −27.588 | 79.795 | −62.911 | 1.00 | 57.15 | |
| ATOM | 166 | CD1 | ILE | | 167 | −26.852 | 79.362 | −64.166 | 1.00 | 55.95 | |
| ATOM | 167 | C | ILE | | 167 | −29.760 | 82.732 | −64.108 | 1.00 | 54.34 | |
| ATOM | 168 | O | ILE | | 167 | −30.172 | 83.522 | −63.263 | 1.00 | 54.22 | |
| ATOM | 169 | N | LEU | | 168 | −30.388 | 82.487 | −65.244 | 1.00 | 53.57 | |
| ATOM | 170 | CA | LEU | | 168 | −31.652 | 83.110 | −65.553 | 1.00 | 54.21 | |
| ATOM | 171 | CB | LEU | | 168 | −31.786 | 83.307 | −67.068 | 1.00 | 53.94 | |
| ATOM | 172 | CG | LEU | | 168 | −33.073 | 83.985 | −67.557 | 1.00 | 54.53 | |
| ATOM | 173 | CD1 | LEU | | 168 | −33.264 | 85.300 | −66.829 | 1.00 | 52.96 | |
| ATOM | 174 | CD2 | LEU | | 168 | −33.015 | 84.203 | −69.064 | 1.00 | 53.85 | |
| ATOM | 175 | C | LEU | | 168 | −32.682 | 82.106 | −65.058 | 1.00 | 54.62 | |
| ATOM | 176 | O | LEU | | 168 | −32.767 | 80.996 | −65.579 | 1.00 | 55.68 | |
| ATOM | 177 | N | VAL | | 169 | −33.458 | 82.479 | −64.048 | 1.00 | 55.53 | |
| ATOM | 178 | CA | VAL | | 169 | −34.471 | 81.562 | −63.510 | 1.00 | 56.35 | |
| ATOM | 179 | CB | VAL | | 169 | −34.188 | 81.185 | −62.028 | 1.00 | 55.31 | |
| ATOM | 180 | CG1 | VAL | | 169 | −32.849 | 80.448 | −61.907 | 1.00 | 52.24 | |
| ATOM | 181 | CG2 | VAL | | 169 | −34.199 | 82.439 | −61.167 | 1.00 | 50.35 | |
| ATOM | 182 | C | VAL | | 169 | −35.885 | 82.114 | −63.537 | 1.00 | 58.43 | |
| ATOM | 183 | O | VAL | | 169 | −36.111 | 83.292 | −63.802 | 1.00 | 57.56 | |
| ATOM | 184 | N | ARG | | 170 | −36.842 | 81.248 | −63.243 | 1.00 | 61.25 | |
| ATOM | 185 | CA | ARG | | 170 | −38.222 | 81.675 | −63.186 | 1.00 | 63.27 | |
| ATOM | 186 | CB | ARG | | 170 | −39.033 | 81.161 | −64.371 | 1.00 | 65.66 | |
| ATOM | 187 | CG | ARG | | 170 | −40.430 | 81.795 | −64.434 | 1.00 | 68.73 | |
| ATOM | 188 | CD | ARG | | 170 | −41.401 | 80.882 | −65.136 | 1.00 | 71.75 | |
| ATOM | 189 | NE | ARG | | 170 | −40.715 | 80.173 | −66.203 | 1.00 | 74.18 | |
| ATOM | 190 | CZ | ARG | | 170 | −40.838 | 78.870 | −66.411 | 1.00 | 76.99 | |
| ATOM | 191 | NH1 | ARG | | 170 | −41.629 | 78.148 | −65.620 | 1.00 | 78.12 | |
| ATOM | 192 | NH2 | ARG | | 170 | −40.158 | 78.287 | −67.393 | 1.00 | 77.33 | |
| ATOM | 193 | C | ARG | | 170 | −38.839 | 81.145 | −61.920 | 1.00 | 63.88 | |
| ATOM | 194 | O | ARG | | 170 | −38.826 | 79.943 | −61.681 | 1.00 | 62.83 | |
| ATOM | 195 | N | GLU | | 171 | −39.368 | 82.058 | −61.117 | 1.00 | 65.09 | |
| ATOM | 196 | CA | GLU | | 171 | −40.030 | 81.724 | −59.868 | 1.00 | 66.62 | |
| ATOM | 197 | CB | GLU | | 171 | −40.279 | 83.011 | −59.069 | 1.00 | 66.66 | |
| ATOM | 198 | CG | GLU | | 171 | −41.025 | 82.839 | −57.743 | 1.00 | 68.07 | |
| ATOM | 199 | CD | GLU | | 171 | −41.262 | 84.170 | −57.029 | 1.00 | 68.86 | |
| ATOM | 200 | OE1 | GLU | | 171 | −41.790 | 85.098 | −57.677 | 1.00 | 69.85 | |
| ATOM | 201 | OE2 | GLU | | 171 | −40.930 | 84.300 | −55.831 | 1.00 | 67.30 | |
| ATOM | 202 | C | GLU | | 171 | −41.356 | 81.047 | −60.215 | 1.00 | 67.69 | |
| ATOM | 203 | O | GLU | | 171 | −42.271 | 81.695 | −60.708 | 1.00 | 67.97 | |
| ATOM | 204 | N | LYS | | 172 | −41.449 | 79.744 | −59.966 | 1.00 | 68.76 | |
| ATOM | 205 | CA | LYS | | 172 | −42.663 | 78.980 | −60.253 | 1.00 | 69.57 | |
| ATOM | 206 | CB | LYS | | 172 | −42.498 | 77.547 | −59.751 | 1.00 | 68.09 | |
| ATOM | 207 | CG | LYS | | 172 | −41.690 | 76.689 | −60.690 | 1.00 | 69.02 | |
| ATOM | 208 | CD | LYS | | 172 | −41.285 | 75.359 | −60.075 | 1.00 | 68.14 | |
| ATOM | 209 | CE | LYS | | 172 | −40.239 | 75.554 | −58.997 | 1.00 | 69.50 | |
| ATOM | 210 | NZ | LYS | | 172 | −39.596 | 74.269 | −58.604 | 1.00 | 68.90 | |
| ATOM | 211 | C | LYS | | 172 | −43.947 | 79.574 | −59.669 | 1.00 | 70.67 | |
| ATOM | 212 | O | LYS | | 172 | −45.036 | 79.401 | −60.221 | 1.00 | 70.71 | |
| ATOM | 213 | N | ALA | | 173 | −43.818 | 80.284 | −58.557 | 1.00 | 72.24 | |
| ATOM | 214 | CA | ALA | | 173 | −44.975 | 80.877 | −57.895 | 1.00 | 73.96 | |
| ATOM | 215 | CB | ALA | | 173 | −44.610 | 81.276 | −56.461 | 1.00 | 74.11 | |
| ATOM | 216 | C | ALA | | 173 | −45.557 | 82.077 | −58.631 | 1.00 | 74.82 | |
| ATOM | 217 | O | ALA | | 173 | −46.768 | 82.284 | −58.619 | 1.00 | 75.00 | |
| ATOM | 218 | N | THR | | 174 | −44.707 | 82.862 | −59.280 | 1.00 | 75.03 | |
| ATOM | 219 | CA | THR | | 174 | −45.194 | 84.042 | −59.968 | 1.00 | 74.48 | |
| ATOM | 220 | CB | THR | | 174 | −44.458 | 85.293 | −59.481 | 1.00 | 74.95 | |
| ATOM | 221 | OG1 | THR | | 174 | −43.072 | 85.187 | −59.829 | 1.00 | 75.75 | |
| ATOM | 222 | CG2 | THR | | 174 | −44.589 | 85.434 | −57.969 | 1.00 | 74.21 | |
| ATOM | 223 | C | THR | | 174 | −45.031 | 83.962 | −61.467 | 1.00 | 74.15 | |
| ATOM | 224 | O | THR | | 174 | −45.641 | 84.736 | −62.200 | 1.00 | 75.49 | |
| ATOM | 225 | N | GLY | | 175 | −44.217 | 83.023 | −61.929 | 1.00 | 73.18 | |
| ATOM | 226 | CA | GLY | | 175 | −43.975 | 82.911 | −63.355 | 1.00 | 70.33 | |
| ATOM | 227 | C | GLY | | 175 | −43.052 | 84.045 | −63.763 | 1.00 | 69.05 | |
| ATOM | 228 | O | GLY | | 175 | −42.743 | 84.212 | −64.937 | 1.00 | 69.75 | |
| ATOM | 229 | N | ARG | | 176 | −42.610 | 84.823 | −62.779 | 1.00 | 67.92 | |
| ATOM | 230 | CA | ARG | | 176 | −41.719 | 85.960 | −62.999 | 1.00 | 68.93 | |
| ATOM | 231 | CB | ARG | | 176 | −41.817 | 86.915 | −61.811 | 1.00 | 71.59 | |
| ATOM | 232 | CG | ARG | | 176 | −43.150 | 87.625 | −61.671 | 1.00 | 75.57 | |
| ATOM | 233 | CD | ARG | | 176 | −43.366 | 88.603 | −62.812 | 1.00 | 79.75 | |
| ATOM | 234 | NE | ARG | | 176 | −44.674 | 89.255 | −62.739 | 1.00 | 84.37 | |
| ATOM | 235 | CZ | ARG | | 176 | −45.225 | 89.958 | −63.730 | 1.00 | 86.47 | |
| ATOM | 236 | NH1 | ARG | | 176 | −44.583 | 90.108 | −64.886 | 1.00 | 87.26 | |
| ATOM | 237 | NH2 | ARG | | 176 | −46.425 | 90.508 | −63.568 | 1.00 | 87.79 | |
| ATOM | 238 | C | ARG | | 176 | −40.242 | 85.576 | −63.212 | 1.00 | 67.74 | |
| ATOM | 239 | O | ARG | | 176 | −39.745 | 84.623 | −62.620 | 1.00 | 67.72 | |
| ATOM | 240 | N | TYR | | 177 | −39.537 | 86.337 | −64.044 | 1.00 | 66.26 | |
| ATOM | 241 | CA | TYR | | 177 | −38.132 | 86.061 | −64.319 | 1.00 | 63.67 | |
| ATOM | 242 | CB | TYR | | 177 | −37.806 | 86.317 | −65.784 | 1.00 | 64.16 | |
| ATOM | 243 | CG | TYR | | 177 | −38.470 | 85.356 | −66.723 | 1.00 | 64.07 | |
| ATOM | 244 | CD1 | TYR | | 177 | −39.806 | 85.502 | −67.075 | 1.00 | 64.56 | |
| ATOM | 245 | CE1 | TYR | | 177 | −40.433 | 84.590 | −67.920 | 1.00 | 65.12 | |
| ATOM | 246 | CD2 | TYR | | 177 | −37.776 | 84.284 | −67.235 | 1.00 | 64.52 | |
| ATOM | 247 | CE2 | TYR | | 177 | −38.395 | 83.372 | −68.076 | 1.00 | 66.54 | |
| ATOM | 248 | CZ | TYR | | 177 | −39.718 | 83.528 | −68.411 | 1.00 | 65.13 | |
| ATOM | 249 | OH | TYR | | 177 | −40.316 | 82.598 | −69.220 | 1.00 | 63.95 | |
| ATOM | 250 | C | TYR | | 177 | −37.160 | 86.854 | −63.476 | 1.00 | 62.79 | |
| ATOM | 251 | O | TYR | | 177 | −37.361 | 88.039 | −63.198 | 1.00 | 62.55 | |
| ATOM | 252 | N | TYR | | 178 | −36.092 | 86.179 | −63.074 | 1.00 | 61.19 | |
| ATOM | 253 | CA | TYR | | 178 | −35.052 | 86.798 | −62.277 | 1.00 | 60.06 | |
| ATOM | 254 | CB | TYR | | 178 | −35.244 | 86.527 | −60.782 | 1.00 | 61.97 | |
| ATOM | 255 | CG | TYR | | 178 | −36.565 | 86.987 | −60.216 | 1.00 | 63.36 | |
| ATOM | 256 | CD1 | TYR | | 178 | −37.740 | 86.288 | −60.491 | 1.00 | 62.38 | |
| ATOM | 257 | CE1 | TYR | | 178 | −38.958 | 86.708 | −59.985 | 1.00 | 63.50 | |
| ATOM | 258 | CD2 | TYR | | 178 | −36.642 | 88.129 | −59.411 | 1.00 | 63.43 | |
| ATOM | 259 | CE2 | TYR | | 178 | −37.866 | 88.563 | −58.897 | 1.00 | 64.13 | |
| ATOM | 260 | CZ | TYR | | 178 | −39.017 | 87.843 | −59.194 | 1.00 | 64.76 | |
| ATOM | 261 | OH | TYR | | 178 | −40.235 | 88.258 | −58.713 | 1.00 | 67.74 | |
| ATOM | 262 | C | TYR | | 178 | −33.714 | 86.237 | −62.675 | 1.00 | 58.06 | |
| ATOM | 263 | O | TYR | | 178 | −33.610 | 85.228 | −63.370 | 1.00 | 56.48 | |
| ATOM | 264 | N | ALA | | 179 | −32.685 | 86.926 | −62.232 | 1.00 | 57.38 | |
| ATOM | 265 | CA | ALA | | 179 | −31.340 | 86.484 | −62.473 | 1.00 | 56.62 | |
| ATOM | 266 | CB | ALA | | 179 | −30.493 | 87.626 | −62.991 | 1.00 | 56.58 | |
| ATOM | 267 | C | ALA | | 179 | −30.876 | 86.055 | −61.087 | 1.00 | 55.18 | |
| ATOM | 268 | O | ALA | | 179 | −31.051 | 86.789 | −60.112 | 1.00 | 53.35 | |
| ATOM | 269 | N | MET | | 180 | −30.325 | 84.851 | −60.999 | 1.00 | 55.43 | |
| ATOM | 270 | CA | MET | | 180 | −29.838 | 84.332 | −59.724 | 1.00 | 55.24 | |
| ATOM | 271 | CB | MET | | 180 | −30.410 | 82.940 | −59.446 | 1.00 | 53.54 | |
| ATOM | 272 | CG | MET | | 180 | −30.001 | 82.356 | −58.100 | 1.00 | 54.72 | |
| ATOM | 273 | SD | MET | | 180 | −30.551 | 80.638 | −57.860 | 1.00 | 54.86 | |
| ATOM | 274 | CE | MET | | 180 | −32.238 | 80.891 | −57.326 | 1.00 | 52.28 | |
| ATOM | 275 | C | MET | | 180 | −28.323 | 84.263 | −59.741 | 1.00 | 55.40 | |
| ATOM | 276 | O | MET | | 180 | −27.718 | 83.596 | −60.585 | 1.00 | 55.12 | |
| ATOM | 277 | N | LYS | | 181 | −27.727 | 84.983 | −58.804 | 1.00 | 56.75 | |
| ATOM | 278 | CA | LYS | | 181 | −26.287 | 85.039 | −58.657 | 1.00 | 58.97 | |
| ATOM | 279 | CB | LYS | | 181 | −25.882 | 86.432 | −58.186 | 1.00 | 60.57 | |
| ATOM | 280 | CG | LYS | | 181 | −24.399 | 86.712 | −58.255 | 1.00 | 66.33 | |
| ATOM | 281 | CD | LYS | | 181 | −24.120 | 88.222 | −58.086 | 1.00 | 70.83 | |
| ATOM | 282 | CE | LYS | | 181 | −22.663 | 88.570 | −58.410 | 1.00 | 72.71 | |
| ATOM | 283 | NZ | LYS | | 181 | −22.345 | 90.004 | −58.147 | 1.00 | 73.68 | |
| ATOM | 284 | C | LYS | | 181 | −25.929 | 83.990 | −57.611 | 1.00 | 58.69 | |
| ATOM | 285 | O | LYS | | 181 | −26.406 | 84.048 | −56.475 | 1.00 | 58.27 | |
| ATOM | 286 | N | ILE | | 182 | −25.105 | 83.027 | −58.011 | 1.00 | 59.91 | |
| ATOM | 287 | CA | ILE | | 182 | −24.678 | 81.933 | −57.133 | 1.00 | 61.37 | |
| ATOM | 288 | CB | ILE | | 182 | −24.906 | 80.567 | −57.818 | 1.00 | 60.20 | |
| ATOM | 289 | CG2 | ILE | | 182 | −24.475 | 79.443 | −56.906 | 1.00 | 60.60 | |
| ATOM | 290 | CG1 | ILE | | 182 | −26.385 | 80.407 | −58.156 | 1.00 | 59.22 | |
| ATOM | 291 | CD1 | ILE | | 182 | −26.687 | 79.161 | −58.947 | 1.00 | 57.26 | |
| ATOM | 292 | C | ILE | | 182 | −23.209 | 82.068 | −56.745 | 1.00 | 62.16 | |
| ATOM | 293 | O | ILE | | 182 | −22.309 | 81.687 | −57.495 | 1.00 | 61.70 | |
| ATOM | 294 | N | LEU | | 183 | −22.980 | 82.614 | −55.559 | 1.00 | 64.42 | |
| ATOM | 295 | CA | LEU | | 183 | −21.632 | 82.828 | −55.067 | 1.00 | 68.23 | |
| ATOM | 296 | CB | LEU | | 183 | −21.565 | 84.124 | −54.266 | 1.00 | 67.68 | |
| ATOM | 297 | CG | LEU | | 183 | −21.808 | 85.409 | −55.041 | 1.00 | 68.73 | |
| ATOM | 298 | CD1 | LEU | | 183 | −21.638 | 86.584 | −54.098 | 1.00 | 68.65 | |
| ATOM | 299 | CD2 | LEU | | 183 | −20.835 | 85.500 | −56.220 | 1.00 | 68.73 | |
| ATOM | 300 | C | LEU | | 183 | −21.101 | 81.716 | −54.189 | 1.00 | 70.99 | |
| ATOM | 301 | O | LEU | | 183 | −21.739 | 81.339 | −53.201 | 1.00 | 70.76 | |
| ATOM | 302 | N | ARG | | 184 | −19.924 | 81.210 | −54.543 | 1.00 | 73.48 | |
| ATOM | 303 | CA | ARG | | 184 | −19.283 | 80.175 | −53.753 | 1.00 | 76.54 | |
| ATOM | 304 | CB | ARG | | 184 | −17.967 | 79.743 | −54.397 | 1.00 | 76.87 | |
| ATOM | 305 | CG | ARG | | 184 | −18.041 | 79.414 | −55.881 | 1.00 | 79.16 | |
| ATOM | 306 | CD | ARG | | 184 | −18.383 | 77.946 | −56.135 | 1.00 | 80.34 | |
| ATOM | 307 | NE | ARG | | 184 | −18.284 | 77.570 | −57.554 | 1.00 | 82.37 | |
| ATOM | 308 | CZ | ARG | | 184 | −18.920 | 78.183 | −58.558 | 1.00 | 83.04 | |
| ATOM | 309 | NH1 | ARG | | 184 | −19.718 | 79.228 | −58.331 | 1.00 | 82.96 | |
| ATOM | 310 | NH2 | ARG | | 184 | −18.777 | 77.738 | −59.800 | 1.00 | 82.81 | |
| ATOM | 311 | C | ARG | | 184 | −18.976 | 80.844 | −52.414 | 1.00 | 78.39 | |
| ATOM | 312 | O | ARG | | 184 | −18.497 | 81.977 | −52.381 | 1.00 | 77.88 | |
| ATOM | 313 | N | LYS | | 185 | −19.286 | 80.172 | −51.311 | 1.00 | 81.21 | |
| ATOM | 314 | CA | LYS | | 185 | −18.979 | 80.726 | −49.997 | 1.00 | 83.85 | |
| ATOM | 315 | CB | LYS | | 185 | −19.663 | 79.918 | −48.886 | 1.00 | 82.70 | |
| ATOM | 316 | CG | LYS | | 185 | −21.127 | 80.273 | −48.646 | 1.00 | 82.88 | |
| ATOM | 317 | CD | LYS | | 185 | −21.737 | 79.414 | −47.532 | 1.00 | 82.13 | |
| ATOM | 318 | CE | LYS | | 185 | −23.202 | 79.768 | −47.259 | 1.00 | 80.52 | |
| ATOM | 319 | NZ | LYS | | 185 | −23.902 | 78.757 | −46.406 | 1.00 | 79.61 | |
| ATOM | 320 | C | LYS | | 185 | −17.468 | 80.569 | −49.903 | 1.00 | 86.31 | |
| ATOM | 321 | O | LYS | | 185 | −16.755 | 81.439 | −49.398 | 1.00 | 86.24 | |
| ATOM | 322 | N | GLU | | 186 | −17.004 | 79.436 | −50.425 | 1.00 | 88.89 | |
| ATOM | 323 | CA | GLU | | 186 | −15.595 | 79.076 | −50.457 | 1.00 | 91.04 | |
| ATOM | 324 | CB | GLU | | 186 | −15.429 | 77.838 | −51.331 | 1.00 | 91.80 | |
| ATOM | 325 | CG | GLU | | 186 | −14.030 | 77.303 | −51.396 | 1.00 | 93.01 | |
| ATOM | 326 | CD | GLU | | 186 | −13.991 | 75.916 | −51.987 | 1.00 | 93.49 | |
| ATOM | 327 | OE1 | GLU | | 186 | −14.374 | 75.754 | −53.165 | 1.00 | 92.38 | |
| ATOM | 328 | OE2 | GLU | | 186 | −13.583 | 74.983 | −51.262 | 1.00 | 94.32 | |
| ATOM | 329 | C | GLU | | 186 | −14.759 | 80.226 | −51.003 | 1.00 | 92.26 | |
| ATOM | 330 | O | GLU | | 186 | −13.808 | 80.676 | −50.361 | 1.00 | 93.52 | |
| ATOM | 331 | N | VAL | | 187 | −15.125 | 80.700 | −52.191 | 1.00 | 93.66 | |
| ATOM | 332 | CA | VAL | | 187 | −14.430 | 81.810 | −52.836 | 1.00 | 94.85 | |
| ATOM | 333 | CB | VAL | | 187 | −15.129 | 82.202 | −54.165 | 1.00 | 95.02 | |
| ATOM | 334 | CG1 | VAL | | 187 | −14.700 | 83.605 | −54.597 | 1.00 | 94.99 | |
| ATOM | 335 | CG2 | VAL | | 187 | −14.792 | 81.182 | −55.247 | 1.00 | 94.32 | |
| ATOM | 336 | C | VAL | | 187 | −14.363 | 83.036 | −51.927 | 1.00 | 95.77 | |
| ATOM | 337 | O | VAL | | 187 | −13.284 | 83.575 | −51.691 | 1.00 | 96.57 | |
| ATOM | 338 | N | ILE | | 188 | −15.516 | 83.471 | −51.423 | 1.00 | 96.66 | |
| ATOM | 339 | CA | ILE | | 188 | −15.588 | 84.634 | −50.539 | 1.00 | 97.25 | |
| ATOM | 340 | CB | ILE | | 188 | −17.052 | 85.166 | −50.409 | 1.00 | 97.70 | |
| ATOM | 341 | CG2 | ILE | | 188 | −17.997 | 84.034 | −50.014 | 1.00 | 98.38 | |
| ATOM | 342 | CG1 | ILE | | 188 | −17.108 | 86.302 | −49.381 | 1.00 | 97.42 | |
| ATOM | 343 | CD1 | ILE | | 188 | −18.504 | 86.790 | −49.062 | 1.00 | 96.61 | |
| ATOM | 344 | C | ILE | | 188 | −15.061 | 84.294 | −49.146 | 1.00 | 97.74 | |
| ATOM | 345 | O | ILE | | 188 | −14.751 | 85.183 | −48.345 | 1.00 | 98.04 | |
| ATOM | 346 | N | ALA | | 198 | −18.217 | 91.398 | −48.874 | 1.00 | 112.30 | |
| ATOM | 347 | CA | ALA | | 198 | −19.344 | 92.061 | −48.228 | 1.00 | 112.68 | |
| ATOM | 348 | CB | ALA | | 198 | −18.944 | 92.535 | −46.839 | 1.00 | 111.86 | |
| ATOM | 349 | C | ALA | | 198 | −19.841 | 93.242 | −49.057 | 1.00 | 113.24 | |
| ATOM | 350 | O | ALA | | 198 | −21.048 | 93.456 | −49.184 | 1.00 | 112.83 | |
| ATOM | 351 | N | ALA | | 199 | −18.908 | 94.005 | −49.622 | 1.00 | 114.02 | |
| ATOM | 352 | CA | ALA | | 199 | −19.255 | 95.170 | −50.438 | 1.00 | 114.16 | |
| ATOM | 353 | CB | ALA | | 199 | −17.989 | 95.795 | −51.030 | 1.00 | 113.26 | |
| ATOM | 354 | C | ALA | | 199 | −20.221 | 94.796 | −51.559 | 1.00 | 114.23 | |
| ATOM | 355 | O | ALA | | 199 | −20.779 | 95.669 | −52.224 | 1.00 | 114.58 | |
| ATOM | 356 | N | ALA | | 200 | −20.418 | 93.496 | −51.759 | 1.00 | 114.26 | |
| ATOM | 357 | CA | ALA | | 200 | −21.309 | 93.003 | −52.804 | 1.00 | 114.40 | |
| ATOM | 358 | CB | ALA | | 200 | −20.589 | 91.932 | −53.633 | 1.00 | 114.28 | |
| ATOM | 359 | C | ALA | | 200 | −22.625 | 92.445 | −52.247 | 1.00 | 114.21 | |
| ATOM | 360 | O | ALA | | 200 | −23.589 | 92.244 | −52.988 | 1.00 | 114.12 | |
| ATOM | 361 | N | ALA | | 201 | −22.667 | 92.205 | −50.940 | 1.00 | 113.54 | |
| ATOM | 362 | CA | ALA | | 201 | −23.863 | 91.664 | −50.305 | 1.00 | 112.74 | |
| ATOM | 363 | CB | ALA | | 201 | −23.510 | 90.409 | −49.513 | 1.00 | 112.58 | |
| ATOM | 364 | C | ALA | | 201 | −24.482 | 92.706 | −49.388 | 1.00 | 112.67 | |
| ATOM | 365 | O | ALA | | 201 | −23.796 | 93.623 | −48.936 | 1.00 | 113.41 | |
| ATOM | 366 | N | ALA | | 202 | −25.777 | 92.553 | −49.112 | 1.00 | 111.66 | |
| ATOM | 367 | CA | ALA | | 202 | −26.520 | 93.475 | −48.253 | 1.00 | 109.46 | |
| ATOM | 368 | CB | ALA | | 202 | −25.917 | 93.503 | −46.845 | 1.00 | 109.54 | |
| ATOM | 369 | C | ALA | | 202 | −26.524 | 94.873 | −48.860 | 1.00 | 107.99 | |
| ATOM | 370 | O | ALA | | 202 | −27.585 | 95.470 | −49.053 | 1.00 | 107.70 | |
| ATOM | 371 | N | ALA | | 203 | −25.332 | 95.386 | −49.163 | 1.00 | 106.33 | |
| ATOM | 372 | CA | ALA | | 203 | −25.189 | 96.711 | −49.759 | 1.00 | 104.82 | |
| ATOM | 373 | CB | ALA | | 203 | −23.759 | 96.915 | −50.279 | 1.00 | 104.06 | |
| ATOM | 374 | C | ALA | | 203 | −26.185 | 96.789 | −50.902 | 1.00 | 103.73 | |
| ATOM | 375 | O | ALA | | 203 | −27.143 | 97.558 | −50.849 | 1.00 | 103.87 | |
| ATOM | 376 | N | ALA | | 204 | −25.959 | 95.972 | −51.925 | 1.00 | 102.19 | |
| ATOM | 377 | CA | ALA | | 204 | −26.843 | 95.929 | −53.078 | 1.00 | 100.05 | |
| ATOM | 378 | CB | ALA | | 204 | −26.277 | 94.995 | −54.148 | 1.00 | 100.32 | |
| ATOM | 379 | C | ALA | | 204 | −28.210 | 95.440 | −52.628 | 1.00 | 98.98 | |
| ATOM | 380 | O | ALA | | 204 | −29.234 | 95.845 | −53.185 | 1.00 | 99.48 | |
| ATOM | 381 | N | ALA | | 205 | −28.219 | 94.575 | −51.612 | 1.00 | 96.81 | |
| ATOM | 382 | CA | ALA | | 205 | −29.459 | 94.016 | −51.079 | 1.00 | 93.77 | |
| ATOM | 383 | CB | ALA | | 205 | −29.171 | 93.174 | −49.847 | 1.00 | 93.47 | |
| ATOM | 384 | C | ALA | | 205 | −30.442 | 95.122 | −50.730 | 1.00 | 91.99 | |
| ATOM | 385 | O | ALA | | 205 | −31.663 | 94.956 | −50.847 | 1.00 | 92.30 | |
| ATOM | 386 | N | ALA | | 206 | −29.911 | 96.260 | −50.306 | 1.00 | 88.38 | |
| ATOM | 387 | CA | ALA | | 206 | −30.778 | 97.360 | −49.948 | 1.00 | 84.60 | |
| ATOM | 388 | CB | ALA | | 206 | −30.428 | 97.858 | −48.568 | 1.00 | 85.09 | |
| ATOM | 389 | C | ALA | | 206 | −30.771 | 98.511 | −50.947 | 1.00 | 81.63 | |
| ATOM | 390 | O | ALA | | 206 | −31.811 | 99.146 | −51.150 | 1.00 | 82.52 | |
| ATOM | 391 | N | THR | | 207 | −29.633 | 98.789 | −51.583 | 1.00 | 77.52 | |
| ATOM | 392 | CA | THR | | 207 | −29.610 | 99.901 | −52.537 | 1.00 | 73.18 | |
| ATOM | 393 | CB | THR | | 207 | −28.264 | 100.054 | −53.262 | 1.00 | 73.37 | |
| ATOM | 394 | OG1 | THR | | 207 | −27.940 | 98.830 | −53.934 | 1.00 | 73.63 | |
| ATOM | 395 | CG2 | THR | | 207 | −27.166 | 100.443 | −52.273 | 1.00 | 71.60 | |
| ATOM | 396 | C | THR | | 207 | −30.703 | 99.705 | −53.559 | 1.00 | 69.95 | |
| ATOM | 397 | O | THR | | 207 | −30.618 | 98.872 | −54.460 | 1.00 | 71.26 | |
| ATOM | 398 | N | ARG | | 208 | −31.752 | 100.482 | −53.382 | 1.00 | 66.22 | |
| ATOM | 399 | CA | ARG | | 208 | −32.903 | 100.424 | −54.249 | 1.00 | 62.60 | |
| ATOM | 400 | CB | ARG | | 208 | −34.161 | 100.336 | −53.392 | 1.00 | 65.54 | |
| ATOM | 401 | CG | ARG | | 208 | −35.441 | 100.224 | −54.168 | 1.00 | 71.05 | |
| ATOM | 402 | CD | ARG | | 208 | −35.830 | 98.778 | −54.390 | 1.00 | 74.90 | |
| ATOM | 403 | NE | ARG | | 208 | −37.066 | 98.710 | −55.161 | 1.00 | 78.98 | |
| ATOM | 404 | CZ | ARG | | 208 | −37.885 | 97.664 | −55.192 | 1.00 | 80.71 | |
| ATOM | 405 | NH1 | ARG | | 208 | −37.608 | 96.571 | −54.486 | 1.00 | 81.00 | |
| ATOM | 406 | NH2 | ARG | | 208 | −38.993 | 97.722 | −55.926 | 1.00 | 81.70 | |
| ATOM | 407 | C | ARG | | 208 | −32.904 | 101.707 | −55.061 | 1.00 | 57.41 | |
| ATOM | 408 | O | ARG | | 208 | −32.743 | 102.789 | −54.512 | 1.00 | 57.88 | |
| ATOM | 409 | N | HIS | | 209 | −33.075 | 101.592 | −56.368 | 1.00 | 51.12 | |
| ATOM | 410 | CA | HIS | | 209 | −33.107 | 102.765 | −57.211 | 1.00 | 44.06 | |
| ATOM | 411 | CB | HIS | | 209 | −31.703 | 103.287 | −57.410 | 1.00 | 40.97 | |
| ATOM | 412 | CG | HIS | | 209 | −31.619 | 104.544 | −58.206 | 1.00 | 39.71 | |
| ATOM | 413 | CD2 | HIS | | 209 | −31.229 | 105.796 | −57.852 | 1.00 | 40.44 | |
| ATOM | 414 | ND1 | HIS | | 209 | −31.847 | 104.582 | −59.566 | 1.00 | 38.76 | |
| ATOM | 415 | CE1 | HIS | | 209 | −31.588 | 105.797 | −60.017 | 1.00 | 39.10 | |
| ATOM | 416 | NE2 | HIS | | 209 | −31.209 | 106.552 | −58.999 | 1.00 | 38.77 | |
| ATOM | 417 | C | HIS | | 209 | −33.705 | 102.334 | −58.518 | 1.00 | 43.50 | |
| ATOM | 418 | O | HIS | | 209 | −33.585 | 101.188 | −58.906 | 1.00 | 42.53 | |
| ATOM | 419 | N | PRO | | 210 | −34.404 | 103.253 | −59.200 | 1.00 | 43.01 | |
| ATOM | 420 | CD | PRO | | 210 | −34.750 | 104.591 | −58.677 | 1.00 | 39.86 | |
| ATOM | 421 | CA | PRO | | 210 | −35.051 | 103.018 | −60.486 | 1.00 | 41.13 | |
| ATOM | 422 | CB | PRO | | 210 | −35.417 | 104.427 | −60.947 | 1.00 | 39.59 | |
| ATOM | 423 | CG | PRO | | 210 | −35.777 | 105.085 | −59.669 | 1.00 | 39.43 | |
| ATOM | 424 | C | PRO | | 210 | −34.169 | 102.333 | −61.507 | 1.00 | 41.97 | |
| ATOM | 425 | O | PRO | | 210 | −34.638 | 101.461 | −62.245 | 1.00 | 41.73 | |
| ATOM | 426 | N | PHE | | 211 | −32.908 | 102.736 | −61.574 | 1.00 | 40.52 | |
| ATOM | 427 | CA | PHE | | 211 | −32.040 | 102.155 | −62.569 | 1.00 | 43.90 | |
| ATOM | 428 | CB | PHE | | 211 | −31.304 | 103.275 | −63.278 | 1.00 | 44.90 | |
| ATOM | 429 | CG | PHE | | 211 | −32.213 | 104.384 | −63.724 | 1.00 | 47.06 | |
| ATOM | 430 | CD1 | PHE | | 211 | −33.322 | 104.107 | −64.509 | 1.00 | 45.32 | |
| ATOM | 431 | CD2 | PHE | | 211 | −31.998 | 105.697 | −63.301 | 1.00 | 46.48 | |
| ATOM | 432 | CE1 | PHE | | 211 | −34.209 | 105.119 | −64.862 | 1.00 | 47.55 | |
| ATOM | 433 | CE2 | PHE | | 211 | −32.876 | 106.707 | −63.647 | 1.00 | 44.53 | |
| ATOM | 434 | CZ | PHE | | 211 | −33.985 | 106.418 | −64.427 | 1.00 | 44.53 | |
| ATOM | 435 | C | PHE | | 211 | −31.077 | 101.097 | −62.053 | 1.00 | 45.03 | |
| ATOM | 436 | O | PHE | | 211 | −30.200 | 100.625 | −62.781 | 1.00 | 45.88 | |
| ATOM | 437 | N | LEU | | 212 | −31.234 | 100.716 | −60.796 | 1.00 | 45.41 | |
| ATOM | 438 | CA | LEU | | 212 | −30.387 | 99.677 | −60.259 | 1.00 | 46.32 | |
| ATOM | 439 | CB | LEU | | 212 | −29.823 | 100.097 | −58.906 | 1.00 | 45.21 | |
| ATOM | 440 | CG | LEU | | 212 | −28.689 | 101.126 | −58.969 | 1.00 | 46.08 | |
| ATOM | 441 | CD1 | LEU | | 212 | −28.195 | 101.433 | −57.580 | 1.00 | 42.17 | |
| ATOM | 442 | CD2 | LEU | | 212 | −27.561 | 100.575 | −59.816 | 1.00 | 44.69 | |
| ATOM | 443 | C | LEU | | 212 | −31.204 | 98.398 | −60.139 | 1.00 | 47.20 | |
| ATOM | 444 | O | LEU | | 212 | −32.345 | 98.419 | −59.696 | 1.00 | 46.29 | |
| ATOM | 445 | N | THR | | 213 | −30.628 | 97.293 | −60.589 | 1.00 | 49.03 | |
| ATOM | 446 | CA | THR | | 213 | −31.271 | 95.989 | −60.508 | 1.00 | 50.07 | |
| ATOM | 447 | CB | THR | | 213 | −30.291 | 94.895 | −60.952 | 1.00 | 52.26 | |
| ATOM | 448 | OG1 | THR | | 213 | −29.718 | 95.257 | −62.215 | 1.00 | 52.98 | |
| ATOM | 449 | CG2 | THR | | 213 | −31.006 | 93.559 | −61.087 | 1.00 | 52.62 | |
| ATOM | 450 | C | THR | | 213 | −31.638 | 95.746 | −59.039 | 1.00 | 49.24 | |
| ATOM | 451 | O | THR | | 213 | −30.822 | 95.962 | −58.148 | 1.00 | 49.43 | |
| ATOM | 452 | N | ALA | | 214 | −32.851 | 95.291 | −58.770 | 1.00 | 49.28 | |
| ATOM | 453 | CA | ALA | | 214 | −33.247 | 95.076 | −57.383 | 1.00 | 50.03 | |
| ATOM | 454 | CB | ALA | | 214 | −34.693 | 95.471 | −57.191 | 1.00 | 49.20 | |
| ATOM | 455 | C | ALA | | 214 | −33.050 | 93.654 | −56.913 | 1.00 | 50.38 | |
| ATOM | 456 | O | ALA | | 214 | −33.210 | 92.709 | −57.681 | 1.00 | 50.97 | |
| ATOM | 457 | N | LEU | | 215 | −32.709 | 93.515 | −55.639 | 1.00 | 51.62 | |
| ATOM | 458 | CA | LEU | | 215 | −32.495 | 92.209 | −55.018 | 1.00 | 53.41 | |
| ATOM | 459 | CB | LEU | | 215 | −31.604 | 92.336 | −53.794 | 1.00 | 52.18 | |
| ATOM | 460 | CG | LEU | | 215 | −31.541 | 91.025 | −53.023 | 1.00 | 55.10 | |
| ATOM | 461 | CD1 | LEU | | 215 | −30.601 | 90.071 | −53.751 | 1.00 | 53.62 | |
| ATOM | 462 | CD2 | LEU | | 215 | −31.083 | 91.272 | −51.598 | 1.00 | 52.91 | |
| ATOM | 463 | C | LEU | | 215 | −33.839 | 91.727 | −54.552 | 1.00 | 55.19 | |
| ATOM | 464 | O | LEU | | 215 | −34.366 | 92.261 | −53.601 | 1.00 | 55.13 | |
| ATOM | 465 | N | LYS | | 216 | −34.405 | 90.729 | −55.213 | 1.00 | 58.13 | |
| ATOM | 466 | CA | LYS | | 216 | −35.706 | 90.231 | −54.799 | 1.00 | 60.30 | |
| ATOM | 467 | CB | LYS | | 216 | −36.348 | 89.456 | −55.932 | 1.00 | 61.60 | |
| ATOM | 468 | CG | LYS | | 216 | −37.769 | 89.099 | −55.658 | 1.00 | 65.28 | |
| ATOM | 469 | CD | LYS | | 216 | −38.609 | 90.335 | −55.480 | 1.00 | 66.80 | |
| ATOM | 470 | CE | LYS | | 216 | −40.077 | 89.974 | −55.595 | 1.00 | 69.41 | |
| ATOM | 471 | NZ | LYS | | 216 | −40.406 | 88.781 | −54.766 | 1.00 | 71.09 | |
| ATOM | 472 | C | LYS | | 216 | −35.574 | 89.356 | −53.544 | 1.00 | 60.92 | |
| ATOM | 473 | O | LYS | | 216 | −36.178 | 89.657 | −52.515 | 1.00 | 61.66 | |
| ATOM | 474 | N | TYR | | 217 | −34.798 | 88.279 | −53.617 | 1.00 | 60.52 | |
| ATOM | 475 | CA | TYR | | 217 | −34.590 | 87.440 | −52.437 | 1.00 | 61.22 | |
| ATOM | 476 | CB | TYR | | 217 | −35.256 | 86.070 | −52.539 | 1.00 | 59.98 | |
| ATOM | 477 | CG | TYR | | 217 | −36.693 | 86.089 | −52.944 | 1.00 | 60.82 | |
| ATOM | 478 | CD1 | TYR | | 217 | −37.054 | 85.919 | −54.272 | 1.00 | 58.85 | |
| ATOM | 479 | CE1 | TYR | | 217 | −38.383 | 85.948 | −54.657 | 1.00 | 62.01 | |
| ATOM | 480 | CD2 | TYR | | 217 | −37.697 | 86.290 | −51.997 | 1.00 | 60.67 | |
| ATOM | 481 | CE2 | TYR | | 217 | −39.037 | 86.317 | −52.370 | 1.00 | 60.66 | |
| ATOM | 482 | CZ | TYR | | 217 | −39.372 | 86.144 | −53.698 | 1.00 | 62.10 | |
| ATOM | 483 | OH | TYR | | 217 | −40.692 | 86.126 | −54.072 | 1.00 | 64.17 | |
| ATOM | 484 | C | TYR | | 217 | −33.120 | 87.178 | −52.269 | 1.00 | 61.56 | |
| ATOM | 485 | O | TYR | | 217 | −32.366 | 87.197 | −53.242 | 1.00 | 62.12 | |
| ATOM | 486 | N | ALA | | 218 | −32.733 | 86.923 | −51.026 | 1.00 | 62.02 | |
| ATOM | 487 | CA | ALA | | 218 | −31.363 | 86.595 | −50.676 | 1.00 | 63.28 | |
| ATOM | 488 | CB | ALA | | 218 | −30.697 | 87.751 | −49.955 | 1.00 | 61.87 | |
| ATOM | 489 | C | ALA | | 218 | −31.412 | 85.384 | −49.759 | 1.00 | 63.85 | |
| ATOM | 490 | O | ALA | | 218 | −31.782 | 85.504 | −48.589 | 1.00 | 64.79 | |
| ATOM | 491 | N | PHE | | 219 | −31.070 | 84.216 | −50.293 | 1.00 | 64.05 | |
| ATOM | 492 | CA | PHE | | 219 | −31.041 | 83.008 | −49.483 | 1.00 | 63.40 | |
| ATOM | 493 | CB | PHE | | 219 | −32.173 | 82.039 | −49.860 | 1.00 | 61.42 | |
| ATOM | 494 | CG | PHE | | 219 | −31.980 | 81.343 | −51.169 | 1.00 | 61.44 | |
| ATOM | 495 | CD1 | PHE | | 219 | −32.354 | 81.948 | −52.355 | 1.00 | 60.16 | |
| ATOM | 496 | CD2 | PHE | | 219 | −31.418 | 80.072 | −51.218 | 1.00 | 60.74 | |
| ATOM | 497 | CE1 | PHE | | 219 | −32.173 | 81.296 | −53.577 | 1.00 | 60.41 | |
| ATOM | 498 | CE2 | PHE | | 219 | −31.234 | 79.413 | −52.433 | 1.00 | 61.58 | |
| ATOM | 499 | CZ | PHE | | 219 | −31.611 | 80.024 | −53.614 | 1.00 | 60.89 | |
| ATOM | 500 | C | PHE | | 219 | −29.683 | 82.348 | −49.672 | 1.00 | 65.35 | |
| ATOM | 501 | O | PHE | | 219 | −28.892 | 82.758 | −50.530 | 1.00 | 65.66 | |
| ATOM | 502 | N | GLN | | 220 | −29.396 | 81.343 | −48.857 | 1.00 | 66.57 | |
| ATOM | 503 | CA | GLN | | 220 | −28.125 | 80.660 | −48.971 | 1.00 | 68.74 | |
| ATOM | 504 | CB | GLN | | 220 | −27.131 | 81.209 | −47.943 | 1.00 | 72.76 | |
| ATOM | 505 | CG | GLN | | 220 | −27.559 | 81.061 | −46.486 | 1.00 | 76.92 | |
| ATOM | 506 | CD | GLN | | 220 | −26.375 | 81.159 | −45.524 | 1.00 | 80.40 | |
| ATOM | 507 | OE1 | GLN | | 220 | −25.585 | 82.111 | −45.581 | 1.00 | 82.71 | |
| ATOM | 508 | NE2 | GLN | | 220 | −26.246 | 80.171 | −44.636 | 1.00 | 81.00 | |
| ATOM | 509 | C | GLN | | 220 | −28.248 | 79.160 | −48.796 | 1.00 | 67.66 | |
| ATOM | 510 | O | GLN | | 220 | −29.077 | 78.683 | −48.027 | 1.00 | 66.84 | |
| ATOM | 511 | N | THR | | 221 | −27.427 | 78.421 | −49.536 | 1.00 | 66.83 | |
| ATOM | 512 | CA | THR | | 221 | −27.409 | 76.975 | −49.436 | 1.00 | 64.35 | |
| ATOM | 513 | CB | THR | | 221 | −27.016 | 76.297 | −50.778 | 1.00 | 62.50 | |
| ATOM | 514 | OG1 | THR | | 221 | −25.718 | 76.730 | −51.192 | 1.00 | 59.24 | |
| ATOM | 515 | CG2 | THR | | 221 | −28.007 | 76.645 | −51.856 | 1.00 | 61.99 | |
| ATOM | 516 | C | THR | | 221 | −26.316 | 76.768 | −48.409 | 1.00 | 65.53 | |
| ATOM | 517 | O | THR | | 221 | −25.758 | 77.743 | −47.912 | 1.00 | 66.18 | |
| ATOM | 518 | N | HIS | | 222 | −26.009 | 75.523 | −48.073 | 1.00 | 66.98 | |
| ATOM | 519 | CA | HIS | | 222 | −24.955 | 75.265 | −47.099 | 1.00 | 68.87 | |
| ATOM | 520 | CB | HIS | | 222 | −25.074 | 73.831 | −46.556 | 1.00 | 69.00 | |
| ATOM | 521 | CG | HIS | | 222 | −23.844 | 72.999 | −46.767 | 1.00 | 69.74 | |
| ATOM | 522 | CD2 | HIS | | 222 | −22.959 | 72.471 | −45.886 | 1.00 | 69.08 | |
| ATOM | 523 | ND1 | HIS | | 222 | −23.378 | 72.670 | −48.022 | 1.00 | 68.15 | |
| ATOM | 524 | CE1 | HIS | | 222 | −22.259 | 71.979 | −47.905 | 1.00 | 69.85 | |
| ATOM | 525 | NE2 | HIS | | 222 | −21.982 | 71.843 | −46.619 | 1.00 | 70.10 | |
| ATOM | 526 | C | HIS | | 222 | −23.604 | 75.455 | −47.795 | 1.00 | 69.04 | |
| ATOM | 527 | O | HIS | | 222 | −22.539 | 75.431 | −47.173 | 1.00 | 70.34 | |
| ATOM | 528 | N | ASP | | 223 | −23.657 | 75.658 | −49.098 | 1.00 | 67.92 | |
| ATOM | 529 | CA | ASP | | 223 | −22.445 | 75.808 | −49.878 | 1.00 | 67.17 | |
| ATOM | 530 | CB | ASP | | 223 | −22.479 | 74.806 | −51.043 | 1.00 | 67.71 | |
| ATOM | 531 | CG | ASP | | 223 | −21.118 | 74.538 | −51.619 | 1.00 | 66.74 | |
| ATOM | 532 | OD1 | ASP | | 223 | −21.034 | 74.122 | −52.800 | 1.00 | 67.60 | |
| ATOM | 533 | OD2 | ASP | | 223 | −20.136 | 74.729 | −50.880 | 1.00 | 66.85 | |
| ATOM | 534 | C | ASP | | 223 | −22.257 | 77.210 | −50.444 | 1.00 | 66.50 | |
| ATOM | 535 | O | ASP | | 223 | −21.161 | 77.768 | −50.389 | 1.00 | 66.77 | |
| ATOM | 536 | N | ARG | | 224 | −23.334 | 77.781 | −50.974 | 1.00 | 64.39 | |
| ATOM | 537 | CA | ARG | | 224 | −23.239 | 79.070 | −51.625 | 1.00 | 62.77 | |
| ATOM | 538 | CB | ARG | | 224 | −23.387 | 78.831 | −53.118 | 1.00 | 61.74 | |
| ATOM | 539 | CG | ARG | | 224 | −22.711 | 77.528 | −53.515 | 1.00 | 60.66 | |
| ATOM | 540 | CD | ARG | | 224 | −22.760 | 77.254 | −54.970 | 1.00 | 60.28 | |
| ATOM | 541 | NE | ARG | | 224 | −21.849 | 76.175 | −55.315 | 1.00 | 62.03 | |
| ATOM | 542 | CZ | ARG | | 224 | −21.550 | 75.828 | −56.559 | 1.00 | 63.12 | |
| ATOM | 543 | NH1 | ARG | | 224 | −22.097 | 76.472 | −57.581 | 1.00 | 64.62 | |
| ATOM | 544 | NH2 | ARG | | 224 | −20.683 | 74.853 | −56.790 | 1.00 | 65.79 | |
| ATOM | 545 | C | ARG | | 224 | −24.230 | 80.106 | −51.161 | 1.00 | 63.89 | |
| ATOM | 546 | O | ARG | | 224 | −25.103 | 79.828 | −50.332 | 1.00 | 65.57 | |
| ATOM | 547 | N | LEU | | 225 | −24.070 | 81.316 | −51.690 | 1.00 | 64.38 | |
| ATOM | 548 | CA | LEU | | 225 | −24.958 | 82.430 | −51.375 | 1.00 | 64.19 | |
| ATOM | 549 | CB | LEU | | 225 | −24.155 | 83.675 | −51.006 | 1.00 | 64.97 | |
| ATOM | 550 | CG | LEU | | 225 | −23.191 | 83.535 | −49.825 | 1.00 | 65.82 | |
| ATOM | 551 | CD1 | LEU | | 225 | −22.392 | 84.820 | −49.678 | 1.00 | 66.26 | |
| ATOM | 552 | CD2 | LEU | | 225 | −23.964 | 83.235 | −48.543 | 1.00 | 65.86 | |
| ATOM | 553 | C | LEU | | 225 | −25.776 | 82.692 | −52.632 | 1.00 | 63.53 | |
| ATOM | 554 | O | LEU | | 225 | −25.238 | 82.692 | −53.748 | 1.00 | 63.44 | |
| ATOM | 555 | N | CYS | | 226 | −27.075 | 82.900 | −52.461 | 1.00 | 62.16 | |
| ATOM | 556 | CA | CYS | | 226 | −27.925 | 83.132 | −53.613 | 1.00 | 61.86 | |
| ATOM | 557 | CB | CYS | | 226 | −28.878 | 81.962 | −53.805 | 1.00 | 61.51 | |
| ATOM | 558 | SG | CYS | | 226 | −27.987 | 80.479 | −54.257 | 1.00 | 67.89 | |
| ATOM | 559 | C | CYS | | 226 | −28.695 | 84.432 | −53.568 | 1.00 | 60.26 | |
| ATOM | 560 | O | CYS | | 226 | −29.355 | 84.766 | −52.586 | 1.00 | 60.82 | |
| ATOM | 561 | N | PHE | | 227 | −28.592 | 85.167 | −54.663 | 1.00 | 58.36 | |
| ATOM | 562 | CA | PHE | | 227 | −29.264 | 86.438 | −54.782 | 1.00 | 56.02 | |
| ATOM | 563 | CB | PHE | | 227 | −28.227 | 87.539 | −54.846 | 1.00 | 55.72 | |
| ATOM | 564 | CG | PHE | | 227 | −27.247 | 87.499 | −53.708 | 1.00 | 55.63 | |
| ATOM | 565 | CD1 | PHE | | 227 | −26.140 | 86.659 | −53.757 | 1.00 | 56.15 | |
| ATOM | 566 | CD2 | PHE | | 227 | −27.424 | 88.304 | −52.594 | 1.00 | 54.88 | |
| ATOM | 567 | CE1 | PHE | | 227 | −25.220 | 86.628 | −52.715 | 1.00 | 55.00 | |
| ATOM | 568 | CE2 | PHE | | 227 | −26.507 | 88.279 | −51.545 | 1.00 | 56.51 | |
| ATOM | 569 | CZ | PHE | | 227 | −25.403 | 87.441 | −51.609 | 1.00 | 55.60 | |
| ATOM | 570 | C | PHE | | 227 | −30.134 | 86.431 | −56.016 | 1.00 | 54.03 | |
| ATOM | 571 | O | PHE | | 227 | −29.658 | 86.369 | −57.142 | 1.00 | 52.89 | |
| ATOM | 572 | N | VAL | | 228 | −31.429 | 86.449 | −55.781 | 1.00 | 52.44 | |
| ATOM | 573 | CA | VAL | | 228 | −32.372 | 86.445 | −56.865 | 1.00 | 52.04 | |
| ATOM | 574 | CB | VAL | | 228 | −33.651 | 85.726 | −56.445 | 1.00 | 51.79 | |
| ATOM | 575 | CG1 | VAL | | 228 | −34.632 | 85.715 | −57.578 | 1.00 | 51.78 | |
| ATOM | 576 | CG2 | VAL | | 228 | −33.314 | 84.310 | −56.017 | 1.00 | 53.18 | |
| ATOM | 577 | C | VAL | | 228 | −32.649 | 87.911 | −57.110 | 1.00 | 52.53 | |
| ATOM | 578 | O | VAL | | 228 | −33.119 | 88.616 | −56.212 | 1.00 | 51.60 | |
| ATOM | 579 | N | MET | | 229 | −32.333 | 88.391 | −58.307 | 1.00 | 53.03 | |
| ATOM | 580 | CA | MET | | 229 | −32.569 | 89.799 | −58.592 | 1.00 | 53.34 | |
| ATOM | 581 | CB | MET | | 229 | −31.242 | 90.536 | −58.712 | 1.00 | 56.21 | |
| ATOM | 582 | CG | MET | | 229 | −30.322 | 90.228 | −57.557 | 1.00 | 61.27 | |
| ATOM | 583 | SD | MET | | 229 | −28.661 | 90.744 | −57.952 | 1.00 | 73.99 | |
| ATOM | 584 | CE | MET | | 229 | −28.454 | 90.022 | −59.617 | 1.00 | 68.07 | |
| ATOM | 585 | C | MET | | 229 | −33.409 | 89.997 | −59.828 | 1.00 | 51.78 | |
| ATOM | 586 | O | MET | | 229 | −33.460 | 89.146 | −60.715 | 1.00 | 50.70 | |
| ATOM | 587 | N | GLU | | 230 | −34.087 | 91.131 | −59.864 | 1.00 | 53.04 | |
| ATOM | 588 | CA | GLU | | 230 | −34.952 | 91.462 | −60.979 | 1.00 | 54.78 | |
| ATOM | 589 | CB | GLU | | 230 | −35.549 | 92.868 | −60.782 | 1.00 | 57.38 | |
| ATOM | 590 | CG | GLU | | 230 | −34.583 | 93.987 | −61.149 | 1.00 | 61.76 | |
| ATOM | 591 | CD | GLU | | 230 | −35.195 | 95.382 | −61.083 | 1.00 | 62.98 | |
| ATOM | 592 | OE1 | GLU | | 230 | −36.266 | 95.603 | −61.690 | 1.00 | 64.36 | |
| ATOM | 593 | OE2 | GLU | | 230 | −34.586 | 96.260 | −60.431 | 1.00 | 64.01 | |
| ATOM | 594 | C | GLU | | 230 | −34.168 | 91.400 | −62.293 | 1.00 | 52.88 | |
| ATOM | 595 | O | GLU | | 230 | −32.988 | 91.775 | −62.352 | 1.00 | 48.54 | |
| ATOM | 596 | N | TYR | | 231 | −34.831 | 90.918 | −63.338 | 1.00 | 52.60 | |
| ATOM | 597 | CA | TYR | | 231 | −34.205 | 90.826 | −64.637 | 1.00 | 53.84 | |
| ATOM | 598 | CB | TYR | | 231 | −34.105 | 89.368 | −65.092 | 1.00 | 54.55 | |
| ATOM | 599 | CG | TYR | | 231 | −33.307 | 89.190 | −66.376 | 1.00 | 56.70 | |
| ATOM | 600 | CD1 | TYR | | 231 | −31.947 | 89.516 | −66.431 | 1.00 | 55.49 | |
| ATOM | 601 | CE1 | TYR | | 231 | −31.205 | 89.327 | −67.608 | 1.00 | 57.10 | |
| ATOM | 602 | CD2 | TYR | | 231 | −33.909 | 88.678 | −67.530 | 1.00 | 58.25 | |
| ATOM | 603 | CE2 | TYR | | 231 | −33.181 | 88.487 | −68.706 | 1.00 | 57.41 | |
| ATOM | 604 | CZ | TYR | | 231 | −31.832 | 88.808 | −68.745 | 1.00 | 57.81 | |
| ATOM | 605 | OH | TYR | | 231 | −31.115 | 88.598 | −69.918 | 1.00 | 59.03 | |
| ATOM | 606 | C | TYR | | 231 | −34.929 | 91.625 | −65.717 | 1.00 | 54.12 | |
| ATOM | 607 | O | TYR | | 231 | −36.075 | 91.332 | −66.069 | 1.00 | 55.07 | |
| ATOM | 608 | N | ALA | | 232 | −34.230 | 92.623 | −66.248 | 1.00 | 53.26 | |
| ATOM | 609 | CA | ALA | | 232 | −34.739 | 93.472 | −67.319 | 1.00 | 54.44 | |
| ATOM | 610 | CB | ALA | | 232 | −33.718 | 94.546 | −67.640 | 1.00 | 51.99 | |
| ATOM | 611 | C | ALA | | 232 | −34.965 | 92.599 | −68.542 | 1.00 | 55.27 | |
| ATOM | 612 | O | ALA | | 232 | −34.065 | 91.856 | −68.943 | 1.00 | 56.46 | |
| ATOM | 613 | N | ASN | | 233 | −36.146 | 92.682 | −69.146 | 1.00 | 55.12 | |
| ATOM | 614 | CA | ASN | | 233 | −36.401 | 91.856 | −70.318 | 1.00 | 55.88 | |
| ATOM | 615 | CB | ASN | | 233 | −37.672 | 91.025 | −70.113 | 1.00 | 58.18 | |
| ATOM | 616 | CG | ASN | | 233 | −38.899 | 91.879 | −69.978 | 1.00 | 56.33 | |
| ATOM | 617 | OD1 | ASN | | 233 | −39.520 | 92.223 | −70.963 | 1.00 | 56.92 | |
| ATOM | 618 | ND2 | ASN | | 233 | −39.239 | 92.247 | −68.747 | 1.00 | 58.52 | |
| ATOM | 619 | C | ASN | | 233 | −36.486 | 92.633 | −71.625 | 1.00 | 55.79 | |
| ATOM | 620 | O | ASN | | 233 | −37.086 | 92.167 | −72.589 | 1.00 | 56.57 | |
| ATOM | 621 | N | GLY | | 234 | −35.879 | 93.816 | −71.654 | 1.00 | 55.96 | |
| ATOM | 622 | CA | GLY | | 234 | −35.874 | 94.619 | −72.865 | 1.00 | 54.78 | |
| ATOM | 623 | C | GLY | | 234 | −34.549 | 94.503 | −73.616 | 1.00 | 55.90 | |
| ATOM | 624 | O | GLY | | 234 | −34.223 | 95.348 | −74.462 | 1.00 | 55.67 | |
| ATOM | 625 | N | GLY | | 235 | −33.773 | 93.461 | −73.307 | 1.00 | 53.62 | |
| ATOM | 626 | CA | GLY | | 235 | −32.505 | 93.266 | −73.981 | 1.00 | 50.27 | |
| ATOM | 627 | C | GLY | | 235 | −31.361 | 94.058 | −73.399 | 1.00 | 49.88 | |
| ATOM | 628 | O | GLY | | 235 | −31.562 | 94.983 | −72.611 | 1.00 | 48.36 | |
| ATOM | 629 | N | GLU | | 236 | −30.147 | 93.692 | −73.789 | 1.00 | 50.89 | |
| ATOM | 630 | CA | GLU | | 236 | −28.952 | 94.368 | −73.296 | 1.00 | 52.45 | |
| ATOM | 631 | CB | GLU | | 236 | −27.805 | 93.369 | −73.108 | 1.00 | 55.02 | |
| ATOM | 632 | CG | GLU | | 236 | −27.858 | 92.179 | −74.044 | 1.00 | 61.22 | |
| ATOM | 633 | CD | GLU | | 236 | −28.458 | 90.912 | −73.408 | 1.00 | 65.08 | |
| ATOM | 634 | OE1 | GLU | | 236 | −29.513 | 90.428 | −73.896 | 1.00 | 67.00 | |
| ATOM | 635 | OE2 | GLU | | 236 | −27.863 | 90.397 | −72.432 | 1.00 | 66.21 | |
| ATOM | 636 | C | GLU | | 236 | −28.508 | 95.484 | −74.217 | 1.00 | 51.78 | |
| ATOM | 637 | O | GLU | | 236 | −28.652 | 95.395 | −75.439 | 1.00 | 50.86 | |
| ATOM | 638 | N | LEU | | 237 | −27.964 | 96.538 | −73.619 | 1.00 | 52.51 | |
| ATOM | 639 | CA | LEU | | 237 | −27.508 | 97.679 | −74.380 | 1.00 | 54.32 | |
| ATOM | 640 | CB | LEU | | 237 | −26.891 | 98.726 | −73.467 | 1.00 | 53.44 | |
| ATOM | 641 | CG | LEU | | 237 | −27.775 | 99.944 | −73.202 | 1.00 | 53.88 | |
| ATOM | 642 | CD1 | LEU | | 237 | −26.850 | 101.115 | −72.909 | 1.00 | 53.26 | |
| ATOM | 643 | CD2 | LEU | | 237 | −28.676 | 100.274 | −74.409 | 1.00 | 51.94 | |
| ATOM | 644 | C | LEU | | 237 | −26.523 | 97.319 | −75.475 | 1.00 | 55.07 | |
| ATOM | 645 | O | LEU | | 237 | −26.622 | 97.824 | −76.591 | 1.00 | 56.10 | |
| ATOM | 646 | N | PHE | | 238 | −25.566 | 96.460 | −75.158 | 1.00 | 57.14 | |
| ATOM | 647 | CA | PHE | | 238 | −24.589 | 96.048 | −76.151 | 1.00 | 58.66 | |
| ATOM | 648 | CB | PHE | | 238 | −23.763 | 94.887 | −75.608 | 1.00 | 63.00 | |
| ATOM | 649 | CG | PHE | | 238 | −23.122 | 94.064 | −76.680 | 1.00 | 68.23 | |
| ATOM | 650 | CD1 | PHE | | 238 | −22.068 | 94.585 | −77.441 | 1.00 | 70.20 | |
| ATOM | 651 | CD2 | PHE | | 238 | −23.608 | 92.791 | −76.978 | 1.00 | 69.62 | |
| ATOM | 652 | CE1 | PHE | | 238 | −21.504 | 93.854 | −78.487 | 1.00 | 71.02 | |
| ATOM | 653 | CE2 | PHE | | 238 | −23.057 | 92.044 | −78.023 | 1.00 | 71.85 | |
| ATOM | 654 | CZ | PHE | | 238 | −22.000 | 92.577 | −78.782 | 1.00 | 72.24 | |
| ATOM | 655 | C | PHE | | 238 | −25.326 | 95.627 | −77.433 | 1.00 | 58.33 | |
| ATOM | 656 | O | PHE | | 238 | −25.061 | 96.144 | −78.523 | 1.00 | 58.19 | |
| ATOM | 657 | N | PHE | | 239 | −26.272 | 94.706 | −77.301 | 1.00 | 57.11 | |
| ATOM | 658 | CA | PHE | | 239 | −27.031 | 94.249 | −78.458 | 1.00 | 56.89 | |
| ATOM | 659 | CB | PHE | | 239 | −28.108 | 93.239 | −78.014 | 1.00 | 59.51 | |
| ATOM | 660 | CG | PHE | | 239 | −29.150 | 92.922 | −79.082 | 1.00 | 62.72 | |
| ATOM | 661 | CD1 | PHE | | 239 | −28.826 | 92.140 | −80.199 | 1.00 | 63.57 | |
| ATOM | 662 | CD2 | PHE | | 239 | −30.464 | 93.412 | −78.966 | 1.00 | 63.29 | |
| ATOM | 663 | CE1 | PHE | | 239 | −29.796 | 91.849 | −81.188 | 1.00 | 63.48 | |
| ATOM | 664 | CE2 | PHE | | 239 | −31.439 | 93.130 | −79.945 | 1.00 | 64.34 | |
| ATOM | 665 | CZ | PHE | | 239 | −31.102 | 92.346 | −81.058 | 1.00 | 64.06 | |
| ATOM | 666 | C | PHE | | 239 | −27.684 | 95.393 | −79.256 | 1.00 | 55.15 | |
| ATOM | 667 | O | PHE | | 239 | −27.582 | 95.416 | −80.488 | 1.00 | 55.59 | |
| ATOM | 668 | N | HIS | | 240 | −28.363 | 96.316 | −78.562 | 1.00 | 52.53 | |
| ATOM | 669 | CA | HIS | | 240 | −29.043 | 97.447 | −79.209 | 1.00 | 50.62 | |
| ATOM | 670 | CB | HIS | | 240 | −29.870 | 98.241 | −78.191 | 1.00 | 49.40 | |
| ATOM | 671 | CG | HIS | | 240 | −31.047 | 97.493 | −77.660 | 1.00 | 44.72 | |
| ATOM | 672 | CD2 | HIS | | 240 | −31.345 | 97.072 | −76.409 | 1.00 | 46.34 | |
| ATOM | 673 | ND1 | HIS | | 240 | −32.058 | 97.032 | −78.472 | 1.00 | 44.43 | |
| ATOM | 674 | CE1 | HIS | | 240 | −32.928 | 96.351 | −77.748 | 1.00 | 44.08 | |
| ATOM | 675 | NE2 | HIS | | 240 | −32.519 | 96.359 | −76.492 | 1.00 | 48.10 | |
| ATOM | 676 | C | HIS | | 240 | −28.031 | 98.379 | −79.861 | 1.00 | 51.18 | |
| ATOM | 677 | O | HIS | | 240 | −28.158 | 98.753 | −81.018 | 1.00 | 51.15 | |
| ATOM | 678 | N | LEU | | 241 | −27.018 | 98.759 | −79.103 | 1.00 | 51.15 | |
| ATOM | 679 | CA | LEU | | 241 | −25.996 | 99.615 | −79.645 | 1.00 | 50.61 | |
| ATOM | 680 | CB | LEU | | 241 | −24.833 | 99.747 | −78.664 | 1.00 | 45.73 | |
| ATOM | 681 | CG | LEU | | 241 | −23.839 | 100.798 | −79.168 | 1.00 | 44.27 | |
| ATOM | 682 | CD1 | LEU | | 241 | −24.560 | 102.140 | −79.451 | 1.00 | 42.06 | |
| ATOM | 683 | CD2 | LEU | | 241 | −22.726 | 100.964 | −78.142 | 1.00 | 43.07 | |
| ATOM | 684 | C | LEU | | 241 | −25.477 | 99.044 | −80.958 | 1.00 | 53.54 | |
| ATOM | 685 | O | LEU | | 241 | −25.604 | 99.673 | −82.016 | 1.00 | 54.97 | |
| ATOM | 686 | N | SER | | 242 | −24.898 | 97.847 | −80.890 | 1.00 | 56.14 | |
| ATOM | 687 | CA | SER | | 242 | −24.335 | 97.214 | −82.075 | 1.00 | 58.25 | |
| ATOM | 688 | CB | SER | | 242 | −23.868 | 95.804 | −81.746 | 1.00 | 57.13 | |
| ATOM | 689 | OG | SER | | 242 | −24.967 | 94.935 | −81.719 | 1.00 | 59.66 | |
| ATOM | 690 | C | SER | | 242 | −25.301 | 97.178 | −83.257 | 1.00 | 59.57 | |
| ATOM | 691 | O | SER | | 242 | −24.866 | 97.190 | −84.401 | 1.00 | 60.46 | |
| ATOM | 692 | N | ARG | | 243 | −26.606 | 97.143 | −82.998 | 1.00 | 61.59 | |
| ATOM | 693 | CA | ARG | | 243 | −27.563 | 97.134 | −84.099 | 1.00 | 64.36 | |
| ATOM | 694 | CB | ARG | | 243 | −28.916 | 96.585 | −83.651 | 1.00 | 66.54 | |
| ATOM | 695 | CG | ARG | | 243 | −28.887 | 95.112 | −83.217 | 1.00 | 71.68 | |
| ATOM | 696 | CD | ARG | | 243 | −30.302 | 94.528 | −83.134 | 1.00 | 73.05 | |
| ATOM | 697 | NE | ARG | | 243 | −30.893 | 94.274 | −84.450 | 1.00 | 76.62 | |
| ATOM | 698 | CZ | ARG | | 243 | −32.146 | 93.875 | −84.642 | 1.00 | 76.35 | |
| ATOM | 699 | NH1 | ARG | | 243 | −32.942 | 93.688 | −83.598 | 1.00 | 76.98 | |
| ATOM | 700 | NH2 | ARG | | 243 | −32.600 | 93.656 | −85.873 | 1.00 | 74.86 | |
| ATOM | 701 | C | ARG | | 243 | −27.774 | 98.525 | −84.679 | 1.00 | 65.42 | |
| ATOM | 702 | O | ARG | | 243 | −28.709 | 98.744 | −85.441 | 1.00 | 66.61 | |
| ATOM | 703 | N | GLU | | 244 | −26.911 | 99.468 | −84.318 | 1.00 | 64.38 | |
| ATOM | 704 | CA | GLU | | 244 | −27.042 | 100.833 | −84.807 | 1.00 | 64.41 | |
| ATOM | 705 | CB | GLU | | 244 | −27.862 | 101.674 | −83.828 | 1.00 | 65.97 | |
| ATOM | 706 | CG | GLU | | 244 | −29.054 | 100.950 | −83.237 | 1.00 | 69.18 | |
| ATOM | 707 | CD | GLU | | 244 | −30.265 | 101.847 | −83.078 | 1.00 | 72.03 | |
| ATOM | 708 | OE1 | GLU | | 244 | −30.913 | 101.795 | −82.005 | 1.00 | 74.67 | |
| ATOM | 709 | OE2 | GLU | | 244 | −30.574 | 102.598 | −84.034 | 1.00 | 73.95 | |
| ATOM | 710 | C | GLU | | 244 | −25.668 | 101.457 | −84.943 | 1.00 | 63.51 | |
| ATOM | 711 | O | GLU | | 244 | −25.534 | 102.582 | −85.435 | 1.00 | 62.57 | |
| ATOM | 712 | N | ARG | | 245 | −24.661 | 100.708 | −84.495 | 1.00 | 61.39 | |
| ATOM | 713 | CA | ARG | | 245 | −23.260 | 101.120 | −84.508 | 1.00 | 60.43 | |
| ATOM | 714 | CB | ARG | | 245 | −22.809 | 101.543 | −85.912 | 1.00 | 61.28 | |
| ATOM | 715 | CG | ARG | | 245 | −21.581 | 102.453 | −85.911 | 1.00 | 64.32 | |
| ATOM | 716 | CD | ARG | | 245 | −20.242 | 101.748 | −85.697 | 1.00 | 67.77 | |
| ATOM | 717 | NE | ARG | | 245 | −19.673 | 101.274 | −86.959 | 1.00 | 71.50 | |
| ATOM | 718 | CZ | ARG | | 245 | −19.872 | 100.058 | −87.470 | 1.00 | 73.03 | |
| ATOM | 719 | NH1 | ARG | | 245 | −20.622 | 99.165 | −86.825 | 1.00 | 72.40 | |
| ATOM | 720 | NH2 | ARG | | 245 | −19.340 | 99.741 | −88.647 | 1.00 | 72.90 | |
| ATOM | 721 | C | ARG | | 245 | −23.058 | 102.260 | −83.524 | 1.00 | 58.48 | |
| ATOM | 722 | O | ARG | | 245 | −22.064 | 102.301 | −82.801 | 1.00 | 58.83 | |
| ATOM | 723 | N | VAL | | 246 | −23.998 | 103.196 | −83.513 | 1.00 | 56.29 | |
| ATOM | 724 | CA | VAL | | 246 | −23.924 | 104.309 | −82.591 | 1.00 | 54.40 | |
| ATOM | 725 | CB | VAL | | 246 | −23.131 | 105.506 | −83.150 | 1.00 | 54.63 | |
| ATOM | 726 | CG1 | VAL | | 246 | −22.577 | 106.312 | −82.016 | 1.00 | 55.47 | |
| ATOM | 727 | CG2 | VAL | | 246 | −22.008 | 105.054 | −84.036 | 1.00 | 57.36 | |
| ATOM | 728 | C | VAL | | 246 | −25.320 | 104.799 | −82.247 | 1.00 | 52.78 | |
| ATOM | 729 | O | VAL | | 246 | −26.304 | 104.487 | −82.920 | 1.00 | 49.96 | |
| ATOM | 730 | N | PHE | | 247 | −25.379 | 105.550 | −81.164 | 1.00 | 50.53 | |
| ATOM | 731 | CA | PHE | | 247 | −26.614 | 106.118 | −80.690 | 1.00 | 50.80 | |
| ATOM | 732 | CB | PHE | | 247 | −26.774 | 105.906 | −79.180 | 1.00 | 49.27 | |
| ATOM | 733 | CG | PHE | | 247 | −27.254 | 104.559 | −78.792 | 1.00 | 46.81 | |
| ATOM | 734 | CD1 | PHE | | 247 | −27.982 | 103.784 | −79.673 | 1.00 | 48.42 | |
| ATOM | 735 | CD2 | PHE | | 247 | −27.037 | 104.092 | −77.512 | 1.00 | 46.55 | |
| ATOM | 736 | CE1 | PHE | | 247 | −28.493 | 102.556 | −79.277 | 1.00 | 47.03 | |
| ATOM | 737 | CE2 | PHE | | 247 | −27.544 | 102.863 | −77.103 | 1.00 | 47.99 | |
| ATOM | 738 | CZ | PHE | | 247 | −28.275 | 102.095 | −77.992 | 1.00 | 47.33 | |
| ATOM | 739 | C | PHE | | 247 | −26.542 | 107.623 | −80.918 | 1.00 | 51.95 | |
| ATOM | 740 | O | PHE | | 247 | −25.461 | 108.223 | −80.874 | 1.00 | 50.17 | |
| ATOM | 741 | N | THR | | 248 | −27.703 | 108.224 | −81.138 | 1.00 | 52.44 | |
| ATOM | 742 | CA | THR | | 248 | −27.809 | 109.669 | −81.297 | 1.00 | 55.16 | |
| ATOM | 743 | CB | THR | | 248 | −29.254 | 110.050 | −81.510 | 1.00 | 55.99 | |
| ATOM | 744 | OG1 | THR | | 248 | −29.546 | 109.992 | −82.910 | 1.00 | 58.05 | |
| ATOM | 745 | CG2 | THR | | 248 | −29.539 | 111.419 | −80.939 | 1.00 | 58.66 | |
| ATOM | 746 | C | THR | | 248 | −27.317 | 110.312 | −80.007 | 1.00 | 53.97 | |
| ATOM | 747 | O | THR | | 248 | −27.478 | 109.745 | −78.940 | 1.00 | 53.89 | |
| ATOM | 748 | N | GLU | | 249 | −26.710 | 111.484 | −80.093 | 1.00 | 54.54 | |
| ATOM | 749 | CA | GLU | | 249 | −26.229 | 112.116 | −78.881 | 1.00 | 56.50 | |
| ATOM | 750 | CB | GLU | | 249 | −25.561 | 113.442 | −79.195 | 1.00 | 55.39 | |
| ATOM | 751 | CG | GLU | | 249 | −24.268 | 113.279 | −79.920 | 1.00 | 58.70 | |
| ATOM | 752 | CD | GLU | | 249 | −23.445 | 114.543 | −79.909 | 1.00 | 60.05 | |
| ATOM | 753 | OE1 | GLU | | 249 | −23.380 | 115.180 | −78.841 | 1.00 | 62.42 | |
| ATOM | 754 | OE2 | GLU | | 249 | −22.852 | 114.895 | −80.951 | 1.00 | 60.76 | |
| ATOM | 755 | C | GLU | | 249 | −27.350 | 112.319 | −77.874 | 1.00 | 57.15 | |
| ATOM | 756 | O | GLU | | 249 | −27.111 | 112.420 | −76.678 | 1.00 | 57.59 | |
| ATOM | 757 | N | GLU | | 250 | −28.579 | 112.378 | −78.344 | 1.00 | 57.29 | |
| ATOM | 758 | CA | GLU | | 250 | −29.665 | 112.558 | −77.408 | 1.00 | 59.02 | |
| ATOM | 759 | CB | GLU | | 250 | −30.881 | 113.184 | −78.083 | 1.00 | 60.37 | |
| ATOM | 760 | CG | GLU | | 250 | −30.920 | 114.707 | −77.961 | 1.00 | 63.15 | |
| ATOM | 761 | CD | GLU | | 250 | −30.865 | 115.198 | −76.505 | 1.00 | 63.92 | |
| ATOM | 762 | OE1 | GLU | | 250 | −29.747 | 115.503 | −76.015 | 1.00 | 62.09 | |
| ATOM | 763 | OE2 | GLU | | 250 | −31.942 | 115.268 | −75.853 | 1.00 | 63.53 | |
| ATOM | 764 | C | GLU | | 250 | −30.043 | 111.237 | −76.777 | 1.00 | 58.15 | |
| ATOM | 765 | O | GLU | | 250 | −30.572 | 111.210 | −75.665 | 1.00 | 59.23 | |
| ATOM | 766 | N | ARG | | 251 | −29.794 | 110.147 | −77.492 | 1.00 | 55.14 | |
| ATOM | 767 | CA | ARG | | 251 | −30.075 | 108.829 | −76.956 | 1.00 | 51.68 | |
| ATOM | 768 | CB | ARG | | 251 | −29.975 | 107.771 | −78.046 | 1.00 | 54.80 | |
| ATOM | 769 | CG | ARG | | 251 | −31.236 | 106.944 | −78.221 | 1.00 | 55.47 | |
| ATOM | 770 | CD | ARG | | 251 | −30.932 | 105.468 | −78.046 | 1.00 | 57.40 | |
| ATOM | 771 | NE | ARG | | 251 | −31.620 | 104.635 | −79.030 | 1.00 | 60.32 | |
| ATOM | 772 | CZ | ARG | | 251 | −31.667 | 103.303 | −78.996 | 1.00 | 61.07 | |
| ATOM | 773 | NH1 | ARG | | 251 | −31.069 | 102.631 | −78.023 | 1.00 | 61.67 | |
| ATOM | 774 | NH2 | ARG | | 251 | −32.309 | 102.637 | −79.944 | 1.00 | 61.79 | |
| ATOM | 775 | C | ARG | | 251 | −28.985 | 108.612 | −75.914 | 1.00 | 48.74 | |
| ATOM | 776 | O | ARG | | 251 | −29.267 | 108.281 | −74.770 | 1.00 | 48.32 | |
| ATOM | 777 | N | ALA | | 252 | −27.734 | 108.817 | −76.296 | 1.00 | 46.02 | |
| ATOM | 778 | CA | ALA | | 252 | −26.654 | 108.642 | −75.333 | 1.00 | 46.47 | |
| ATOM | 779 | CB | ALA | | 252 | −25.376 | 109.340 | −75.800 | 1.00 | 44.07 | |
| ATOM | 780 | C | ALA | | 252 | −27.183 | 109.324 | −74.089 | 1.00 | 45.73 | |
| ATOM | 781 | O | ALA | | 252 | −27.374 | 108.699 | −73.044 | 1.00 | 47.17 | |
| ATOM | 782 | N | ALA | | 253 | −27.421 | 110.622 | −74.240 | 1.00 | 43.78 | |
| ATOM | 783 | CA | ALA | | 253 | −27.948 | 111.491 | −73.199 | 1.00 | 38.33 | |
| ATOM | 784 | CB | ALA | | 253 | −28.493 | 112.720 | −73.840 | 1.00 | 37.71 | |
| ATOM | 785 | C | ALA | | 253 | −29.034 | 110.815 | −72.396 | 1.00 | 35.92 | |
| ATOM | 786 | O | ALA | | 253 | −28.969 | 110.708 | −71.181 | 1.00 | 37.83 | |
| ATOM | 787 | N | PHE | | 254 | −30.054 | 110.336 | −73.065 | 1.00 | 33.51 | |
| ATOM | 788 | CA | PHE | | 254 | −31.092 | 109.718 | −72.304 | 1.00 | 35.15 | |
| ATOM | 789 | CB | PHE | | 254 | −32.128 | 109.129 | −73.202 | 1.00 | 36.77 | |
| ATOM | 790 | CG | PHE | | 254 | −33.197 | 108.427 | −72.455 | 1.00 | 43.38 | |
| ATOM | 791 | CD1 | PHE | | 254 | −34.253 | 109.143 | −71.903 | 1.00 | 42.17 | |
| ATOM | 792 | CD2 | PHE | | 254 | −33.144 | 107.041 | −72.287 | 1.00 | 44.22 | |
| ATOM | 793 | CE1 | PHE | | 254 | −35.261 | 108.493 | −71.192 | 1.00 | 44.09 | |
| ATOM | 794 | CE2 | PHE | | 254 | −34.140 | 106.367 | −71.581 | 1.00 | 46.95 | |
| ATOM | 795 | CZ | PHE | | 254 | −35.212 | 107.098 | −71.028 | 1.00 | 46.80 | |
| ATOM | 796 | C | PHE | | 254 | −30.555 | 108.618 | −71.398 | 1.00 | 38.34 | |
| ATOM | 797 | O | PHE | | 254 | −30.915 | 108.574 | −70.227 | 1.00 | 39.59 | |
| ATOM | 798 | N | TYR | | 255 | −29.710 | 107.726 | −71.932 | 1.00 | 38.81 | |
| ATOM | 799 | CA | TYR | | 255 | −29.138 | 106.622 | −71.128 | 1.00 | 37.31 | |
| ATOM | 800 | CB | TYR | | 255 | −28.457 | 105.555 | −72.016 | 1.00 | 33.21 | |
| ATOM | 801 | CG | TYR | | 255 | −29.437 | 104.850 | −72.934 | 1.00 | 32.18 | |
| ATOM | 802 | CD1 | TYR | | 255 | −30.628 | 104.364 | −72.445 | 1.00 | 31.36 | |
| ATOM | 803 | CE1 | TYR | | 255 | −31.548 | 103.785 | −73.283 | 1.00 | 34.88 | |
| ATOM | 804 | CD2 | TYR | | 255 | −29.191 | 104.726 | −74.299 | 1.00 | 32.34 | |
| ATOM | 805 | CE2 | TYR | | 255 | −30.117 | 104.144 | −75.144 | 1.00 | 29.63 | |
| ATOM | 806 | CZ | TYR | | 255 | −31.286 | 103.681 | −74.627 | 1.00 | 31.05 | |
| ATOM | 807 | OH | TYR | | 255 | −32.218 | 103.100 | −75.431 | 1.00 | 34.60 | |
| ATOM | 808 | C | TYR | | 255 | −28.145 | 107.113 | −70.096 | 1.00 | 36.47 | |
| ATOM | 809 | O | TYR | | 255 | −28.168 | 106.687 | −68.950 | 1.00 | 40.42 | |
| ATOM | 810 | N | GLY | | 256 | −27.278 | 108.028 | −70.479 | 1.00 | 35.21 | |
| ATOM | 811 | CA | GLY | | 256 | −26.302 | 108.482 | −69.521 | 1.00 | 33.66 | |
| ATOM | 812 | C | GLY | | 256 | −26.908 | 109.108 | −68.291 | 1.00 | 38.33 | |
| ATOM | 813 | O | GLY | | 256 | −26.409 | 108.899 | −67.172 | 1.00 | 41.37 | |
| ATOM | 814 | N | ALA | | 257 | −27.970 | 109.894 | −68.477 | 1.00 | 36.89 | |
| ATOM | 815 | CA | ALA | | 257 | −28.599 | 110.570 | −67.354 | 1.00 | 34.76 | |
| ATOM | 816 | CB | ALA | | 257 | −29.688 | 111.565 | −67.862 | 1.00 | 36.88 | |
| ATOM | 817 | C | ALA | | 257 | −29.205 | 109.567 | −66.361 | 1.00 | 33.00 | |
| ATOM | 818 | O | ALA | | 257 | −29.072 | 109.749 | −65.170 | 1.00 | 35.08 | |
| ATOM | 819 | N | GLU | | 258 | −29.864 | 108.520 | −66.830 | 1.00 | 33.02 | |
| ATOM | 820 | CA | GLU | | 258 | −30.424 | 107.563 | −65.896 | 1.00 | 36.92 | |
| ATOM | 821 | CB | GLU | | 258 | −31.248 | 106.489 | −66.627 | 1.00 | 38.50 | |
| ATOM | 822 | CG | GLU | | 258 | −32.366 | 107.082 | −67.499 | 1.00 | 38.85 | |
| ATOM | 823 | CD | GLU | | 258 | −33.535 | 106.114 | −67.769 | 1.00 | 44.40 | |
| ATOM | 824 | OE1 | GLU | | 258 | −33.271 | 104.923 | −68.061 | 1.00 | 45.03 | |
| ATOM | 825 | OE2 | GLU | | 258 | −34.728 | 106.540 | −67.698 | 1.00 | 43.40 | |
| ATOM | 826 | C | GLU | | 258 | −29.249 | 106.949 | −65.099 | 1.00 | 39.65 | |
| ATOM | 827 | O | GLU | | 258 | −29.373 | 106.663 | −63.893 | 1.00 | 43.01 | |
| ATOM | 828 | N | ILE | | 259 | −28.097 | 106.815 | −65.749 | 1.00 | 36.89 | |
| ATOM | 829 | CA | ILE | | 259 | −26.929 | 106.268 | −65.085 | 1.00 | 36.09 | |
| ATOM | 830 | CB | ILE | | 259 | −25.794 | 105.895 | −66.118 | 1.00 | 35.08 | |
| ATOM | 831 | CG2 | ILE | | 259 | −24.466 | 105.704 | −65.416 | 1.00 | 36.36 | |
| ATOM | 832 | CG1 | ILE | | 259 | −26.170 | 104.624 | −66.868 | 1.00 | 33.56 | |
| ATOM | 833 | CD1 | ILE | | 259 | −25.203 | 104.279 | −68.021 | 1.00 | 36.34 | |
| ATOM | 834 | C | ILE | | 259 | −26.414 | 107.273 | −64.079 | 1.00 | 35.91 | |
| ATOM | 835 | O | ILE | | 259 | −26.079 | 106.918 | −62.944 | 1.00 | 36.63 | |
| ATOM | 836 | N | VAL | | 260 | −26.335 | 108.535 | −64.475 | 1.00 | 36.67 | |
| ATOM | 837 | CA | VAL | | 260 | −25.849 | 109.551 | −63.537 | 1.00 | 34.46 | |
| ATOM | 838 | CB | VAL | | 260 | −25.840 | 110.964 | −64.156 | 1.00 | 32.53 | |
| ATOM | 839 | CG1 | VAL | | 260 | −25.309 | 111.951 | −63.137 | 1.00 | 30.91 | |
| ATOM | 840 | CG2 | VAL | | 260 | −24.933 | 110.996 | −65.353 | 1.00 | 26.79 | |
| ATOM | 841 | C | VAL | | 260 | −26.694 | 109.594 | −62.279 | 1.00 | 33.98 | |
| ATOM | 842 | O | VAL | | 260 | −26.186 | 109.803 | −61.164 | 1.00 | 33.13 | |
| ATOM | 843 | N | SER | | 261 | −27.988 | 109.371 | −62.473 | 1.00 | 35.21 | |
| ATOM | 844 | CA | SER | | 261 | −28.961 | 109.363 | −61.375 | 1.00 | 36.44 | |
| ATOM | 845 | CB | SER | | 261 | −30.367 | 109.154 | −61.957 | 1.00 | 34.12 | |
| ATOM | 846 | OG | SER | | 261 | −31.335 | 109.138 | −60.941 | 1.00 | 37.38 | |
| ATOM | 847 | C | SER | | 261 | −28.618 | 108.261 | −60.351 | 1.00 | 36.60 | |
| ATOM | 848 | O | SER | | 261 | −28.585 | 108.499 | −59.123 | 1.00 | 34.41 | |
| ATOM | 849 | N | ALA | | 262 | −28.356 | 107.061 | −60.875 | 1.00 | 36.24 | |
| ATOM | 850 | CA | ALA | | 262 | −27.988 | 105.898 | −60.055 | 1.00 | 36.35 | |
| ATOM | 851 | CB | ALA | | 262 | −27.822 | 104.679 | −60.928 | 1.00 | 37.72 | |
| ATOM | 852 | C | ALA | | 262 | −26.701 | 106.178 | −59.317 | 1.00 | 35.05 | |
| ATOM | 853 | O | ALA | | 262 | −26.626 | 106.041 | −58.106 | 1.00 | 39.79 | |
| ATOM | 854 | N | LEU | | 263 | −25.687 | 106.591 | −60.049 | 1.00 | 36.70 | |
| ATOM | 855 | CA | LEU | | 263 | −24.406 | 106.889 | −59.433 | 1.00 | 37.91 | |
| ATOM | 856 | CB | LEU | | 263 | −23.385 | 107.255 | −60.522 | 1.00 | 35.37 | |
| ATOM | 857 | CG | LEU | | 263 | −22.971 | 106.076 | −61.423 | 1.00 | 38.91 | |
| ATOM | 858 | CD1 | LEU | | 263 | −21.907 | 106.537 | −62.445 | 1.00 | 33.32 | |
| ATOM | 859 | CD2 | LEU | | 263 | −22.422 | 104.934 | −60.572 | 1.00 | 34.51 | |
| ATOM | 860 | C | LEU | | 263 | −24.468 | 107.978 | −58.348 | 1.00 | 38.63 | |
| ATOM | 861 | O | LEU | | 263 | −23.790 | 107.870 | −57.313 | 1.00 | 40.82 | |
| ATOM | 862 | N | GLU | | 264 | −25.243 | 109.033 | −58.585 | 1.00 | 39.29 | |
| ATOM | 863 | CA | GLU | | 264 | −25.380 | 110.108 | −57.591 | 1.00 | 39.52 | |
| ATOM | 864 | CB | GLU | | 264 | −26.394 | 111.162 | −58.051 | 1.00 | 43.65 | |
| ATOM | 865 | CG | GLU | | 264 | −26.881 | 112.048 | −56.899 | 1.00 | 49.73 | |
| ATOM | 866 | CD | GLU | | 264 | −27.767 | 113.216 | −57.346 | 1.00 | 52.93 | |
| ATOM | 867 | OE1 | GLU | | 264 | −28.758 | 113.004 | −58.078 | 1.00 | 54.86 | |
| ATOM | 868 | OE2 | GLU | | 264 | −27.472 | 114.356 | −56.941 | 1.00 | 54.54 | |
| ATOM | 869 | C | GLU | | 264 | −25.906 | 109.485 | −56.305 | 1.00 | 37.10 | |
| ATOM | 870 | O | GLU | | 264 | −25.491 | 109.830 | −55.197 | 1.00 | 32.98 | |
| ATOM | 871 | N | TYR | | 265 | −26.834 | 108.555 | −56.493 | 1.00 | 37.40 | |
| ATOM | 872 | CA | TYR | | 265 | −27.465 | 107.861 | −55.406 | 1.00 | 37.88 | |
| ATOM | 873 | CB | TYR | | 265 | −28.609 | 106.993 | −55.918 | 1.00 | 43.44 | |
| ATOM | 874 | CG | TYR | | 265 | −29.424 | 106.466 | −54.768 | 1.00 | 48.31 | |
| ATOM | 875 | CD1 | TYR | | 265 | −30.166 | 107.340 | −53.982 | 1.00 | 53.35 | |
| ATOM | 876 | CE1 | TYR | | 265 | −30.876 | 106.899 | −52.863 | 1.00 | 55.97 | |
| ATOM | 877 | CD2 | TYR | | 265 | −29.412 | 105.124 | −54.416 | 1.00 | 50.34 | |
| ATOM | 878 | CE2 | TYR | | 265 | −30.130 | 104.668 | −53.296 | 1.00 | 53.43 | |
| ATOM | 879 | CZ | TYR | | 265 | −30.861 | 105.572 | −52.525 | 1.00 | 55.06 | |
| ATOM | 880 | OH | TYR | | 265 | −31.602 | 105.189 | −51.416 | 1.00 | 59.22 | |
| ATOM | 881 | C | TYR | | 265 | −26.479 | 106.984 | −54.663 | 1.00 | 38.40 | |
| ATOM | 882 | O | TYR | | 265 | −26.371 | 107.069 | −53.440 | 1.00 | 38.68 | |
| ATOM | 883 | N | LEU | | 266 | −25.765 | 106.129 | −55.397 | 1.00 | 36.66 | |
| ATOM | 884 | CA | LEU | | 266 | −24.800 | 105.241 | −54.756 | 1.00 | 34.81 | |
| ATOM | 885 | CB | LEU | | 266 | −24.179 | 104.280 | −55.778 | 1.00 | 33.68 | |
| ATOM | 886 | CG | LEU | | 266 | −25.120 | 103.207 | −56.359 | 1.00 | 34.62 | |
| ATOM | 887 | CD1 | LEU | | 266 | −24.512 | 102.522 | −57.548 | 1.00 | 33.12 | |
| ATOM | 888 | CD2 | LEU | | 266 | −25.439 | 102.176 | −55.286 | 1.00 | 35.25 | |
| ATOM | 889 | C | LEU | | 266 | −23.731 | 106.057 | −54.047 | 1.00 | 32.09 | |
| ATOM | 890 | O | LEU | | 266 | −23.404 | 105.779 | −52.915 | 1.00 | 28.47 | |
| ATOM | 891 | N | HIS | | 267 | −23.227 | 107.105 | −54.678 | 1.00 | 34.14 | |
| ATOM | 892 | CA | HIS | | 267 | −22.190 | 107.871 | −54.008 | 1.00 | 36.06 | |
| ATOM | 893 | CB | HIS | | 267 | −21.556 | 108.895 | −54.954 | 1.00 | 37.44 | |
| ATOM | 894 | CG | HIS | | 267 | −20.822 | 108.265 | −56.099 | 1.00 | 39.63 | |
| ATOM | 895 | CD2 | HIS | | 267 | −20.728 | 106.970 | −56.484 | 1.00 | 37.04 | |
| ATOM | 896 | ND1 | HIS | | 267 | −20.123 | 108.993 | −57.037 | 1.00 | 39.82 | |
| ATOM | 897 | CE1 | HIS | | 267 | −19.637 | 108.173 | −57.954 | 1.00 | 37.38 | |
| ATOM | 898 | NE2 | HIS | | 267 | −19.993 | 106.941 | −57.641 | 1.00 | 36.45 | |
| ATOM | 899 | C | HIS | | 267 | −22.690 | 108.547 | −52.758 | 1.00 | 39.21 | |
| ATOM | 900 | O | HIS | | 267 | −21.916 | 108.743 | −51.818 | 1.00 | 40.44 | |
| ATOM | 901 | N | SER | | 268 | −23.972 | 108.902 | −52.718 | 1.00 | 39.67 | |
| ATOM | 902 | CA | SER | | 268 | −24.492 | 109.539 | −51.514 | 1.00 | 42.53 | |
| ATOM | 903 | CB | SER | | 268 | −25.934 | 110.007 | −51.709 | 1.00 | 42.69 | |
| ATOM | 904 | OG | SER | | 268 | −26.823 | 108.908 | −51.645 | 1.00 | 47.00 | |
| ATOM | 905 | C | SER | | 268 | −24.432 | 108.559 | −50.342 | 1.00 | 42.62 | |
| ATOM | 906 | O | SER | | 268 | −24.422 | 108.976 | −49.196 | 1.00 | 42.09 | |
| ATOM | 907 | N | ARG | | 269 | −24.413 | 107.262 | −50.640 | 1.00 | 45.09 | |
| ATOM | 908 | CA | ARG | | 269 | −24.327 | 106.224 | −49.626 | 1.00 | 48.53 | |
| ATOM | 909 | CB | ARG | | 269 | −25.195 | 105.029 | −50.013 | 1.00 | 51.99 | |
| ATOM | 910 | CG | ARG | | 269 | −26.672 | 105.309 | −50.120 | 1.00 | 57.10 | |
| ATOM | 911 | CD | ARG | | 269 | −27.251 | 105.588 | −48.746 | 1.00 | 62.32 | |
| ATOM | 912 | NE | ARG | | 269 | −28.707 | 105.763 | −48.740 | 1.00 | 67.05 | |
| ATOM | 913 | CZ | ARG | | 269 | −29.373 | 106.699 | −49.424 | 1.00 | 68.97 | |
| ATOM | 914 | NH1 | ARG | | 269 | −28.730 | 107.566 | −50.201 | 1.00 | 69.91 | |
| ATOM | 915 | NH2 | ARG | | 269 | −30.691 | 106.794 | −49.302 | 1.00 | 69.33 | |
| ATOM | 916 | C | ARG | | 269 | −22.877 | 105.734 | −49.505 | 1.00 | 50.09 | |
| ATOM | 917 | O | ARG | | 269 | −22.649 | 104.611 | −49.060 | 1.00 | 49.03 | |
| ATOM | 918 | N | ASP | | 270 | −21.915 | 106.558 | −49.924 | 1.00 | 50.68 | |
| ATOM | 919 | CA | ASP | | 270 | −20.492 | 106.206 | −49.871 | 1.00 | 52.09 | |
| ATOM | 920 | CB | ASP | | 270 | −20.010 | 106.164 | −48.417 | 1.00 | 57.80 | |
| ATOM | 921 | CG | ASP | | 270 | −18.635 | 106.833 | −48.217 | 1.00 | 63.46 | |
| ATOM | 922 | OD1 | ASP | | 270 | −17.708 | 106.571 | −49.030 | 1.00 | 64.87 | |
| ATOM | 923 | OD2 | ASP | | 270 | −18.480 | 107.611 | −47.232 | 1.00 | 66.66 | |
| ATOM | 924 | C | ASP | | 270 | −20.218 | 104.845 | −50.539 | 1.00 | 50.49 | |
| ATOM | 925 | O | ASP | | 270 | −19.346 | 104.084 | −50.116 | 1.00 | 51.23 | |
| ATOM | 926 | N | VAL | | 271 | −20.985 | 104.542 | −51.574 | 1.00 | 45.87 | |
| ATOM | 927 | CA | VAL | | 271 | −20.831 | 103.304 | −52.300 | 1.00 | 44.41 | |
| ATOM | 928 | CB | VAL | | 271 | −22.163 | 102.633 | −52.559 | 1.00 | 42.09 | |
| ATOM | 929 | CG1 | VAL | | 271 | −21.989 | 101.524 | −53.568 | 1.00 | 40.36 | |
| ATOM | 930 | CG2 | VAL | | 271 | −22.721 | 102.114 | −51.276 | 1.00 | 43.19 | |
| ATOM | 931 | C | VAL | | 271 | −20.213 | 103.557 | −53.666 | 1.00 | 46.59 | |
| ATOM | 932 | O | VAL | | 271 | −20.705 | 104.400 | −54.427 | 1.00 | 45.80 | |
| ATOM | 933 | N | VAL | | 272 | −19.150 | 102.810 | −53.974 | 1.00 | 45.19 | |
| ATOM | 934 | CA | VAL | | 272 | −18.469 | 102.930 | −55.253 | 1.00 | 42.21 | |
| ATOM | 935 | CB | VAL | | 272 | −16.957 | 102.802 | −55.093 | 1.00 | 40.51 | |
| ATOM | 936 | CG1 | VAL | | 272 | −16.313 | 102.730 | −56.476 | 1.00 | 42.91 | |
| ATOM | 937 | CG2 | VAL | | 272 | −16.405 | 104.001 | −54.326 | 1.00 | 38.71 | |
| ATOM | 938 | C | VAL | | 272 | −18.983 | 101.788 | −56.100 | 1.00 | 41.41 | |
| ATOM | 939 | O | VAL | | 272 | −18.947 | 100.644 | −55.682 | 1.00 | 42.62 | |
| ATOM | 940 | N | TYR | | 273 | −19.464 | 102.059 | −57.295 | 1.00 | 40.02 | |
| ATOM | 941 | CA | TYR | | 273 | −19.997 | 100.938 | −58.038 | 1.00 | 40.54 | |
| ATOM | 942 | CB | TYR | | 273 | −20.982 | 101.428 | −59.092 | 1.00 | 37.31 | |
| ATOM | 943 | CG | TYR | | 273 | −21.854 | 100.323 | −59.594 | 1.00 | 33.65 | |
| ATOM | 944 | CD1 | TYR | | 273 | −22.811 | 99.729 | −58.770 | 1.00 | 36.03 | |
| ATOM | 945 | CE1 | TYR | | 273 | −23.602 | 98.665 | −59.234 | 1.00 | 37.71 | |
| ATOM | 946 | CD2 | TYR | | 273 | −21.702 | 99.842 | −60.872 | 1.00 | 34.61 | |
| ATOM | 947 | CE2 | TYR | | 273 | −22.479 | 98.794 | −61.345 | 1.00 | 38.18 | |
| ATOM | 948 | CZ | TYR | | 273 | −23.422 | 98.210 | −60.528 | 1.00 | 36.31 | |
| ATOM | 949 | OH | TYR | | 273 | −24.167 | 97.177 | −61.040 | 1.00 | 40.78 | |
| ATOM | 950 | C | TYR | | 273 | −18.962 | 100.001 | −58.695 | 1.00 | 41.21 | |
| ATOM | 951 | O | TYR | | 273 | −19.210 | 98.799 | −58.812 | 1.00 | 39.04 | |
| ATOM | 952 | N | ARG | | 274 | −17.849 | 100.567 | −59.151 | 1.00 | 40.96 | |
| ATOM | 953 | CA | ARG | | 274 | −16.772 | 99.824 | −59.812 | 1.00 | 44.77 | |
| ATOM | 954 | CB | ARG | | 274 | −16.198 | 98.756 | −58.871 | 1.00 | 44.32 | |
| ATOM | 955 | CG | ARG | | 274 | −15.573 | 99.267 | −57.569 | 1.00 | 45.68 | |
| ATOM | 956 | CD | ARG | | 274 | −15.067 | 98.053 | −56.759 | 1.00 | 47.47 | |
| ATOM | 957 | NE | ARG | | 274 | −14.406 | 98.397 | −55.497 | 1.00 | 52.46 | |
| ATOM | 958 | CZ | ARG | | 274 | −13.355 | 97.739 | −55.002 | 1.00 | 51.71 | |
| ATOM | 959 | NH1 | ARG | | 274 | −12.839 | 96.710 | −55.666 | 1.00 | 50.62 | |
| ATOM | 960 | NH2 | ARG | | 274 | −12.827 | 98.099 | −53.838 | 1.00 | 49.56 | |
| ATOM | 961 | C | ARG | | 274 | −17.114 | 99.136 | −61.139 | 1.00 | 45.03 | |
| ATOM | 962 | O | ARG | | 274 | −16.225 | 98.858 | −61.933 | 1.00 | 45.38 | |
| ATOM | 963 | N | ASP | | 275 | −18.391 | 98.860 | −61.384 | 1.00 | 46.86 | |
| ATOM | 964 | CA | ASP | | 275 | −18.796 | 98.160 | −62.605 | 1.00 | 47.22 | |
| ATOM | 965 | CB | ASP | | 275 | −19.534 | 96.872 | −62.227 | 1.00 | 50.91 | |
| ATOM | 966 | CG | ASP | | 275 | −18.600 | 95.798 | −61.674 | 1.00 | 55.90 | |
| ATOM | 967 | OD1 | ASP | | 275 | −17.746 | 96.099 | −60.804 | 1.00 | 55.33 | |
| ATOM | 968 | OD2 | ASP | | 275 | −18.734 | 94.632 | −62.117 | 1.00 | 61.84 | |
| ATOM | 969 | C | ASP | | 275 | −19.629 | 98.898 | −63.649 | 1.00 | 46.70 | |
| ATOM | 970 | O | ASP | | 275 | −20.512 | 98.300 | −64.276 | 1.00 | 46.88 | |
| ATOM | 971 | N | ILE | | 276 | −19.375 | 100.182 | −63.859 | 1.00 | 45.39 | |
| ATOM | 972 | CA | ILE | | 276 | −20.139 | 100.865 | −64.890 | 1.00 | 42.27 | |
| ATOM | 973 | CB | ILE | | 276 | −20.120 | 102.415 | −64.728 | 1.00 | 44.44 | |
| ATOM | 974 | CG2 | ILE | | 276 | −20.910 | 103.055 | −65.867 | 1.00 | 40.81 | |
| ATOM | 975 | CG1 | ILE | | 276 | −20.751 | 102.842 | −63.389 | 1.00 | 41.78 | |
| ATOM | 976 | CD1 | ILE | | 276 | −22.138 | 102.296 | −63.147 | 1.00 | 39.09 | |
| ATOM | 977 | C | ILE | | 276 | −19.501 | 100.521 | −66.225 | 1.00 | 42.12 | |
| ATOM | 978 | O | ILE | | 276 | −18.404 | 100.958 | −66.522 | 1.00 | 41.23 | |
| ATOM | 979 | N | LYS | | 277 | −20.169 | 99.713 | −67.031 | 1.00 | 42.85 | |
| ATOM | 980 | CA | LYS | | 277 | −19.630 | 99.379 | −68.336 | 1.00 | 44.14 | |
| ATOM | 981 | CB | LYS | | 277 | −18.410 | 98.445 | −68.210 | 1.00 | 47.06 | |
| ATOM | 982 | CG | LYS | | 277 | −18.561 | 97.203 | −67.357 | 1.00 | 49.66 | |
| ATOM | 983 | CD | LYS | | 277 | −17.188 | 96.507 | −67.310 | 1.00 | 57.47 | |
| ATOM | 984 | CE | LYS | | 277 | −17.163 | 95.206 | −66.516 | 1.00 | 58.16 | |
| ATOM | 985 | NZ | LYS | | 277 | −17.881 | 94.084 | −67.215 | 1.00 | 61.79 | |
| ATOM | 986 | C | LYS | | 277 | −20.737 | 98.787 | −69.174 | 1.00 | 43.44 | |
| ATOM | 987 | O | LYS | | 277 | −21.646 | 98.178 | −68.644 | 1.00 | 47.32 | |
| ATOM | 988 | N | LEU | | 278 | −20.669 | 98.986 | −70.481 | 1.00 | 42.84 | |
| ATOM | 989 | CA | LEU | | 278 | −21.712 | 98.551 | −71.400 | 1.00 | 42.92 | |
| ATOM | 990 | CB | LEU | | 278 | −21.201 | 98.664 | −72.841 | 1.00 | 45.02 | |
| ATOM | 991 | CG | LEU | | 278 | −22.217 | 98.490 | −73.981 | 1.00 | 49.27 | |
| ATOM | 992 | CD1 | LEU | | 278 | −23.288 | 99.584 | −73.900 | 1.00 | 49.33 | |
| ATOM | 993 | CD2 | LEU | | 278 | −21.495 | 98.562 | −75.347 | 1.00 | 49.44 | |
| ATOM | 994 | C | LEU | | 278 | −22.329 | 97.178 | −71.186 | 1.00 | 45.54 | |
| ATOM | 995 | O | LEU | | 278 | −23.536 | 97.005 | −71.345 | 1.00 | 45.54 | |
| ATOM | 996 | N | GLU | | 279 | −21.508 | 96.194 | −70.834 | 1.00 | 45.98 | |
| ATOM | 997 | CA | GLU | | 279 | −21.993 | 94.832 | −70.625 | 1.00 | 45.74 | |
| ATOM | 998 | CB | GLU | | 279 | −20.795 | 93.898 | −70.397 | 1.00 | 49.69 | |
| ATOM | 999 | CG | GLU | | 279 | −19.574 | 94.156 | −71.318 | 1.00 | 55.11 | |
| ATOM | 1000 | CD | GLU | | 279 | −18.863 | 95.478 | −71.020 | 1.00 | 58.90 | |
| ATOM | 1001 | OE1 | GLU | | 279 | −18.900 | 95.923 | −69.859 | 1.00 | 63.47 | |
| ATOM | 1002 | OE2 | GLU | | 279 | −18.248 | 96.074 | −71.932 | 1.00 | 60.83 | |
| ATOM | 1003 | C | GLU | | 279 | −22.951 | 94.769 | −69.423 | 1.00 | 44.36 | |
| ATOM | 1004 | O | GLU | | 279 | −23.839 | 93.914 | −69.348 | 1.00 | 41.91 | |
| ATOM | 1005 | N | ASN | | 280 | −22.766 | 95.672 | −68.470 | 1.00 | 42.34 | |
| ATOM | 1006 | CA | ASN | | 280 | −23.629 | 95.685 | −67.322 | 1.00 | 42.85 | |
| ATOM | 1007 | CB | ASN | | 280 | −22.851 | 96.205 | −66.120 | 1.00 | 45.62 | |
| ATOM | 1008 | CG | ASN | | 280 | −21.818 | 95.186 | −65.608 | 1.00 | 48.24 | |
| ATOM | 1009 | OD1 | ASN | | 280 | −21.001 | 94.708 | −66.358 | 1.00 | 50.55 | |
| ATOM | 1010 | ND2 | ASN | | 280 | −21.869 | 94.865 | −64.330 | 1.00 | 48.97 | |
| ATOM | 1011 | C | ASN | | 280 | −24.896 | 96.519 | −67.562 | 1.00 | 42.89 | |
| ATOM | 1012 | O | ASN | | 280 | −25.646 | 96.790 | −66.635 | 1.00 | 44.04 | |
| ATOM | 1013 | N | LEU | | 281 | −25.155 | 96.911 | −68.803 | 1.00 | 41.26 | |
| ATOM | 1014 | CA | LEU | | 281 | −26.341 | 97.718 | −69.080 | 1.00 | 40.35 | |
| ATOM | 1015 | CB | LEU | | 281 | −25.993 | 99.011 | −69.842 | 1.00 | 35.92 | |
| ATOM | 1016 | CG | LEU | | 281 | −25.004 | 99.939 | −69.121 | 1.00 | 35.39 | |
| ATOM | 1017 | CD1 | LEU | | 281 | −24.662 | 101.111 | −69.992 | 1.00 | 32.28 | |
| ATOM | 1018 | CD2 | LEU | | 281 | −25.579 | 100.372 | −67.790 | 1.00 | 33.88 | |
| ATOM | 1019 | C | LEU | | 281 | −27.385 | 96.987 | −69.868 | 1.00 | 41.54 | |
| ATOM | 1020 | O | LEU | | 281 | −27.092 | 96.360 | −70.907 | 1.00 | 40.29 | |
| ATOM | 1021 | N | MET | | 282 | −28.609 | 97.074 | −69.355 | 1.00 | 40.75 | |
| ATOM | 1022 | CA | MET | | 282 | −29.765 | 96.481 | −70.002 | 1.00 | 44.50 | |
| ATOM | 1023 | CB | MET | | 282 | −30.222 | 95.240 | −69.235 | 1.00 | 46.49 | |
| ATOM | 1024 | CG | MET | | 282 | −29.137 | 94.171 | −69.092 | 1.00 | 51.07 | |
| ATOM | 1025 | SD | MET | | 282 | −29.909 | 92.558 | −69.088 | 1.00 | 57.33 | |
| ATOM | 1026 | CE | MET | | 282 | −30.611 | 92.576 | −67.423 | 1.00 | 56.55 | |
| ATOM | 1027 | C | MET | | 282 | −30.913 | 97.504 | −70.057 | 1.00 | 45.24 | |
| ATOM | 1028 | O | MET | | 282 | −30.831 | 98.588 | −69.464 | 1.00 | 42.96 | |
| ATOM | 1029 | N | LEU | | 283 | −31.975 | 97.156 | −70.775 | 1.00 | 45.25 | |
| ATOM | 1030 | CA | LEU | | 283 | −33.142 | 98.014 | −70.854 | 1.00 | 45.02 | |
| ATOM | 1031 | CB | LEU | | 283 | −33.456 | 98.374 | −72.290 | 1.00 | 42.75 | |
| ATOM | 1032 | CG | LEU | | 283 | −32.475 | 99.150 | −73.135 | 1.00 | 42.36 | |
| ATOM | 1033 | CD1 | LEU | | 283 | −33.134 | 99.402 | −74.492 | 1.00 | 41.24 | |
| ATOM | 1034 | CD2 | LEU | | 283 | −32.088 | 100.440 | −72.452 | 1.00 | 41.01 | |
| ATOM | 1035 | C | LEU | | 283 | −34.303 | 97.197 | −70.316 | 1.00 | 46.71 | |
| ATOM | 1036 | O | LEU | | 283 | −34.357 | 95.984 | −70.528 | 1.00 | 44.94 | |
| ATOM | 1037 | N | ASP | | 284 | −35.235 | 97.834 | −69.613 | 1.00 | 48.53 | |
| ATOM | 1038 | CA | ASP | | 284 | −36.391 | 97.078 | −69.146 | 1.00 | 50.53 | |
| ATOM | 1039 | CB | ASP | | 284 | −36.951 | 97.624 | −67.822 | 1.00 | 52.33 | |
| ATOM | 1040 | CG | ASP | | 284 | −37.398 | 99.066 | −67.907 | 1.00 | 52.25 | |
| ATOM | 1041 | OD1 | ASP | | 284 | −37.813 | 99.541 | −68.987 | 1.00 | 52.03 | |
| ATOM | 1042 | OD2 | ASP | | 284 | −37.348 | 99.724 | −66.852 | 1.00 | 54.97 | |
| ATOM | 1043 | C | ASP | | 284 | −37.465 | 97.120 | −70.233 | 1.00 | 50.52 | |
| ATOM | 1044 | O | ASP | | 284 | −37.286 | 97.758 | −71.266 | 1.00 | 48.72 | |
| ATOM | 1045 | N | LYS | | 285 | −38.570 | 96.433 | −70.001 | 1.00 | 51.98 | |
| ATOM | 1046 | CA | LYS | | 285 | −39.651 | 96.391 | −70.973 | 1.00 | 54.76 | |
| ATOM | 1047 | CB | LYS | | 285 | −40.889 | 95.751 | −70.338 | 1.00 | 55.91 | |
| ATOM | 1048 | CG | LYS | | 285 | −41.266 | 96.363 | −69.003 | 1.00 | 58.33 | |
| ATOM | 1049 | CD | LYS | | 285 | −42.302 | 95.524 | −68.289 | 1.00 | 60.68 | |
| ATOM | 1050 | CE | LYS | | 285 | −42.334 | 95.866 | −66.809 | 1.00 | 62.11 | |
| ATOM | 1051 | NZ | LYS | | 285 | −41.058 | 95.456 | −66.129 | 1.00 | 62.92 | |
| ATOM | 1052 | C | LYS | | 285 | −40.023 | 97.756 | −71.560 | 1.00 | 54.80 | |
| ATOM | 1053 | O | LYS | | 285 | −40.461 | 97.834 | −72.710 | 1.00 | 55.83 | |
| ATOM | 1054 | N | ASP | | 286 | −39.834 | 98.825 | −70.788 | 1.00 | 54.22 | |
| ATOM | 1055 | CA | ASP | | 286 | −40.203 | 100.160 | −71.247 | 1.00 | 54.53 | |
| ATOM | 1056 | CB | ASP | | 286 | −40.831 | 100.947 | −70.101 | 1.00 | 55.42 | |
| ATOM | 1057 | CG | ASP | | 286 | −42.065 | 100.277 | −69.552 | 1.00 | 56.65 | |
| ATOM | 1058 | OD1 | ASP | | 286 | −42.930 | 99.908 | −70.374 | 1.00 | 58.04 | |
| ATOM | 1059 | OD2 | ASP | | 286 | −42.169 | 100.124 | −68.310 | 1.00 | 56.41 | |
| ATOM | 1060 | C | ASP | | 286 | −39.141 | 101.040 | −71.876 | 1.00 | 54.31 | |
| ATOM | 1061 | O | ASP | | 286 | −39.387 | 102.220 | −72.101 | 1.00 | 58.59 | |
| ATOM | 1062 | N | GLY | | 287 | −37.965 | 100.516 | −72.161 | 1.00 | 51.01 | |
| ATOM | 1063 | CA | GLY | | 287 | −36.962 | 101.378 | −72.753 | 1.00 | 48.45 | |
| ATOM | 1064 | C | GLY | | 287 | −36.064 | 102.006 | −71.714 | 1.00 | 47.14 | |
| ATOM | 1065 | O | GLY | | 287 | −35.092 | 102.673 | −72.066 | 1.00 | 48.27 | |
| ATOM | 1066 | N | HIS | | 288 | −36.378 | 101.803 | −70.435 | 1.00 | 47.24 | |
| ATOM | 1067 | CA | HIS | | 288 | −35.551 | 102.342 | −69.347 | 1.00 | 47.36 | |
| ATOM | 1068 | CB | HIS | | 288 | −36.411 | 102.565 | −68.114 | 1.00 | 47.39 | |
| ATOM | 1069 | CG | HIS | | 288 | −37.181 | 103.840 | −68.161 | 1.00 | 48.56 | |
| ATOM | 1070 | CD2 | HIS | | 288 | −38.513 | 104.078 | −68.209 | 1.00 | 46.05 | |
| ATOM | 1071 | ND1 | HIS | | 288 | −36.565 | 105.073 | −68.166 | 1.00 | 50.60 | |
| ATOM | 1072 | CE1 | HIS | | 288 | −37.488 | 106.019 | −68.211 | 1.00 | 50.41 | |
| ATOM | 1073 | NE2 | HIS | | 288 | −38.677 | 105.441 | −68.236 | 1.00 | 46.30 | |
| ATOM | 1074 | C | HIS | | 288 | −34.309 | 101.490 | −68.972 | 1.00 | 46.61 | |
| ATOM | 1075 | O | HIS | | 288 | −34.331 | 100.252 | −69.031 | 1.00 | 44.63 | |
| ATOM | 1076 | N | ILE | | 289 | −33.234 | 102.173 | −68.581 | 1.00 | 44.71 | |
| ATOM | 1077 | CA | ILE | | 289 | −31.985 | 101.518 | −68.213 | 1.00 | 43.43 | |
| ATOM | 1078 | CB | ILE | | 289 | −30.820 | 102.522 | −68.191 | 1.00 | 43.26 | |
| ATOM | 1079 | CG2 | ILE | | 289 | −30.061 | 102.415 | −66.861 | 1.00 | 42.64 | |
| ATOM | 1080 | CG1 | ILE | | 289 | −29.934 | 102.306 | −69.432 | 1.00 | 43.34 | |
| ATOM | 1081 | CD1 | ILE | | 289 | −28.522 | 102.939 | −69.357 | 1.00 | 46.39 | |
| ATOM | 1082 | C | ILE | | 289 | −31.970 | 100.769 | −66.876 | 1.00 | 44.25 | |
| ATOM | 1083 | O | ILE | | 289 | −32.575 | 101.200 | −65.882 | 1.00 | 44.29 | |
| ATOM | 1084 | N | LYS | | 290 | −31.264 | 99.641 | −66.866 | 1.00 | 42.98 | |
| ATOM | 1085 | CA | LYS | | 290 | −31.123 | 98.833 | −65.666 | 1.00 | 41.38 | |
| ATOM | 1086 | CB | LYS | | 290 | −31.941 | 97.545 | −65.750 | 1.00 | 39.02 | |
| ATOM | 1087 | CG | LYS | | 290 | −33.361 | 97.681 | −65.368 | 1.00 | 40.24 | |
| ATOM | 1088 | CD | LYS | | 290 | −33.484 | 97.938 | −63.870 | 1.00 | 41.83 | |
| ATOM | 1089 | CE | LYS | | 290 | −34.893 | 98.339 | −63.545 | 1.00 | 40.44 | |
| ATOM | 1090 | NZ | LYS | | 290 | −34.963 | 98.747 | −62.141 | 1.00 | 42.96 | |
| ATOM | 1091 | C | LYS | | 290 | −29.674 | 98.457 | −65.552 | 1.00 | 40.99 | |
| ATOM | 1092 | O | LYS | | 290 | −29.194 | 97.656 | −66.319 | 1.00 | 40.05 | |
| ATOM | 1093 | N | ILE | | 291 | −28.979 | 99.053 | −64.597 | 1.00 | 42.46 | |
| ATOM | 1094 | CA | ILE | | 291 | −27.589 | 98.748 | −64.354 | 1.00 | 42.00 | |
| ATOM | 1095 | CB | ILE | | 291 | −26.970 | 99.846 | −63.530 | 1.00 | 41.72 | |
| ATOM | 1096 | CG2 | ILE | | 291 | −25.624 | 99.425 | −63.022 | 1.00 | 39.30 | |
| ATOM | 1097 | CG1 | ILE | | 291 | −26.911 | 101.125 | −64.379 | 1.00 | 41.56 | |
| ATOM | 1098 | CD1 | ILE | | 291 | −26.386 | 102.329 | −63.638 | 1.00 | 40.40 | |
| ATOM | 1099 | C | ILE | | 291 | −27.544 | 97.415 | −63.602 | 1.00 | 45.01 | |
| ATOM | 1100 | O | ILE | | 291 | −28.018 | 97.285 | −62.482 | 1.00 | 40.95 | |
| ATOM | 1101 | N | THR | | 292 | −26.970 | 96.426 | −64.264 | 1.00 | 49.33 | |
| ATOM | 1102 | CA | THR | | 292 | −26.855 | 95.073 | −63.758 | 1.00 | 53.20 | |
| ATOM | 1103 | CB | THR | | 292 | −26.859 | 94.118 | −64.941 | 1.00 | 54.04 | |
| ATOM | 1104 | OG1 | THR | | 292 | −27.852 | 93.116 | −64.747 | 1.00 | 52.48 | |
| ATOM | 1105 | CG2 | THR | | 292 | −25.502 | 93.507 | −65.137 | 1.00 | 53.65 | |
| ATOM | 1106 | C | THR | | 292 | −25.581 | 94.913 | −62.963 | 1.00 | 58.02 | |
| ATOM | 1107 | O | THR | | 292 | −24.707 | 95.773 | −62.990 | 1.00 | 59.64 | |
| ATOM | 1108 | N | ASP | | 293 | −25.469 | 93.801 | −62.257 | 1.00 | 64.16 | |
| ATOM | 1109 | CA | ASP | | 293 | −24.291 | 93.537 | −61.428 | 1.00 | 69.42 | |
| ATOM | 1110 | CB | ASP | | 293 | −24.722 | 93.339 | −59.962 | 1.00 | 72.22 | |
| ATOM | 1111 | CG | ASP | | 293 | −23.631 | 93.708 | −58.971 | 1.00 | 75.85 | |
| ATOM | 1112 | OD1 | ASP | | 293 | −22.477 | 93.246 | −59.147 | 1.00 | 79.23 | |
| ATOM | 1113 | OD2 | ASP | | 293 | −23.927 | 94.461 | −58.010 | 1.00 | 77.35 | |
| ATOM | 1114 | C | ASP | | 293 | −23.582 | 92.273 | −61.909 | 1.00 | 71.22 | |
| ATOM | 1115 | O | ASP | | 293 | −23.439 | 91.323 | −61.140 | 1.00 | 73.99 | |
| ATOM | 1116 | N | PHE | | 294 | −23.149 | 92.250 | −63.167 | 1.00 | 72.25 | |
| ATOM | 1117 | CA | PHE | | 294 | −22.475 | 91.077 | −63.728 | 1.00 | 73.70 | |
| ATOM | 1118 | CB | PHE | | 294 | −23.204 | 89.804 | −63.303 | 1.00 | 72.45 | |
| ATOM | 1119 | CG | PHE | | 294 | −24.660 | 89.793 | −63.672 | 1.00 | 70.33 | |
| ATOM | 1120 | CD1 | PHE | | 294 | −25.061 | 89.453 | −64.959 | 1.00 | 67.51 | |
| ATOM | 1121 | CD2 | PHE | | 294 | −25.636 | 90.131 | −62.727 | 1.00 | 69.26 | |
| ATOM | 1122 | CE1 | PHE | | 294 | −26.420 | 89.447 | −65.306 | 1.00 | 67.84 | |
| ATOM | 1123 | CE2 | PHE | | 294 | −26.998 | 90.127 | −63.063 | 1.00 | 68.34 | |
| ATOM | 1124 | CZ | PHE | | 294 | −27.392 | 89.784 | −64.357 | 1.00 | 67.45 | |
| ATOM | 1125 | C | PHE | | 294 | −22.445 | 91.135 | −65.247 | 1.00 | 75.40 | |
| ATOM | 1126 | O | PHE | | 294 | −23.320 | 91.738 | −65.871 | 1.00 | 76.19 | |
| ATOM | 1127 | N | GLY | | 295 | −21.459 | 90.481 | −65.849 | 1.00 | 76.43 | |
| ATOM | 1128 | CA | GLY | | 295 | −21.371 | 90.504 | −67.295 | 1.00 | 77.74 | |
| ATOM | 1129 | C | GLY | | 295 | −21.782 | 89.222 | −67.991 | 1.00 | 78.56 | |
| ATOM | 1130 | O | GLY | | 295 | −21.533 | 88.125 | −67.498 | 1.00 | 78.40 | |
| ATOM | 1131 | N | ALA | | 296 | −22.408 | 89.370 | −69.155 | 1.00 | 79.85 | |
| ATOM | 1132 | CA | ALA | | 296 | −22.851 | 88.230 | −69.947 | 1.00 | 80.07 | |
| ATOM | 1133 | CB | ALA | | 296 | −23.711 | 88.714 | −71.116 | 1.00 | 78.65 | |
| ATOM | 1134 | C | ALA | | 296 | −21.637 | 87.448 | −70.468 | 1.00 | 80.65 | |
| ATOM | 1135 | O | ALA | | 296 | −21.490 | 86.246 | −70.221 | 1.00 | 79.69 | |
| ATOM | 1136 | N | THR | | 313 | −14.667 | 91.536 | −75.055 | 1.00 | 64.50 | |
| ATOM | 1137 | CA | THR | | 313 | −14.516 | 92.943 | −74.706 | 1.00 | 63.57 | |
| ATOM | 1138 | CB | THR | | 313 | −15.812 | 93.497 | −74.037 | 1.00 | 64.19 | |
| ATOM | 1139 | OG1 | THR | | 313 | −16.126 | 92.731 | −72.865 | 1.00 | 63.91 | |
| ATOM | 1140 | CG2 | THR | | 313 | −16.997 | 93.422 | −75.014 | 1.00 | 65.19 | |
| ATOM | 1141 | C | THR | | 313 | −13.314 | 93.198 | −73.775 | 1.00 | 62.47 | |
| ATOM | 1142 | O | THR | | 313 | −13.105 | 92.490 | −72.789 | 1.00 | 61.71 | |
| ATOM | 1143 | N | PRO | | 314 | −12.481 | 94.191 | −74.114 | 1.00 | 60.78 | |
| ATOM | 1144 | CD | PRO | | 314 | −12.345 | 94.703 | −75.485 | 1.00 | 60.25 | |
| ATOM | 1145 | CA | PRO | | 314 | −11.304 | 94.542 | −73.307 | 1.00 | 60.12 | |
| ATOM | 1146 | CB | PRO | | 314 | −10.421 | 95.313 | −74.292 | 1.00 | 58.86 | |
| ATOM | 1147 | CG | PRO | | 314 | −10.849 | 94.810 | −75.622 | 1.00 | 61.26 | |
| ATOM | 1148 | C | PRO | | 314 | −11.714 | 95.424 | −72.122 | 1.00 | 59.62 | |
| ATOM | 1149 | O | PRO | | 314 | −12.561 | 96.304 | −72.275 | 1.00 | 61.90 | |
| ATOM | 1150 | N | GLU | | 315 | −11.130 | 95.194 | −70.951 | 1.00 | 58.34 | |
| ATOM | 1151 | CA | GLU | | 315 | −11.444 | 96.004 | −69.776 | 1.00 | 57.56 | |
| ATOM | 1152 | CB | GLU | | 315 | −10.955 | 95.337 | −68.494 | 1.00 | 60.10 | |
| ATOM | 1153 | CG | GLU | | 315 | −11.839 | 94.246 | −67.961 | 1.00 | 66.09 | |
| ATOM | 1154 | CD | GLU | | 315 | −11.955 | 94.301 | −66.441 | 1.00 | 68.46 | |
| ATOM | 1155 | OE1 | GLU | | 315 | −12.651 | 95.213 | −65.929 | 1.00 | 69.22 | |
| ATOM | 1156 | OE2 | GLU | | 315 | −11.349 | 93.440 | −65.762 | 1.00 | 69.03 | |
| ATOM | 1157 | C | GLU | | 315 | −10.798 | 97.387 | −69.844 | 1.00 | 56.14 | |
| ATOM | 1158 | O | GLU | | 315 | −9.649 | 97.528 | −70.241 | 1.00 | 54.63 | |
| ATOM | 1159 | N | TYR | | 316 | −11.538 | 98.412 | −69.447 | 1.00 | 52.66 | |
| ATOM | 1160 | CA | TYR | | 316 | −10.987 | 99.746 | −69.452 | 1.00 | 48.79 | |
| ATOM | 1161 | CB | TYR | | 316 | −11.782 | 100.657 | −70.372 | 1.00 | 46.62 | |
| ATOM | 1162 | CG | TYR | | 316 | −11.294 | 100.677 | −71.793 | 1.00 | 43.91 | |
| ATOM | 1163 | CD1 | TYR | | 316 | −11.606 | 99.650 | −72.674 | 1.00 | 41.71 | |
| ATOM | 1164 | CE1 | TYR | | 316 | −11.224 | 99.715 | −74.026 | 1.00 | 43.79 | |
| ATOM | 1165 | CD2 | TYR | | 316 | −10.571 | 101.769 | −72.275 | 1.00 | 44.76 | |
| ATOM | 1166 | CE2 | TYR | | 316 | −10.183 | 101.850 | −73.610 | 1.00 | 43.33 | |
| ATOM | 1167 | CZ | TYR | | 316 | −10.514 | 100.834 | −74.485 | 1.00 | 42.50 | |
| ATOM | 1168 | OH | TYR | | 316 | −10.189 | 100.967 | −75.818 | 1.00 | 43.57 | |
| ATOM | 1169 | C | TYR | | 316 | −11.035 | 100.272 | −68.040 | 1.00 | 48.58 | |
| ATOM | 1170 | O | TYR | | 316 | −11.866 | 101.127 | −67.707 | 1.00 | 49.20 | |
| ATOM | 1171 | N | LEU | | 317 | −10.154 | 99.745 | −67.202 | 1.00 | 45.40 | |
| ATOM | 1172 | CA | LEU | | 317 | −10.113 | 100.178 | −65.825 | 1.00 | 45.80 | |
| ATOM | 1173 | CB | LEU | | 317 | −9.159 | 99.311 | −64.991 | 1.00 | 44.82 | |
| ATOM | 1174 | CG | LEU | | 317 | −9.614 | 97.878 | −64.736 | 1.00 | 47.19 | |
| ATOM | 1175 | CD1 | LEU | | 317 | −8.574 | 97.186 | −63.873 | 1.00 | 46.19 | |
| ATOM | 1176 | CD2 | LEU | | 317 | −10.983 | 97.859 | −64.027 | 1.00 | 44.93 | |
| ATOM | 1177 | C | LEU | | 317 | −9.663 | 101.612 | −65.767 | 1.00 | 45.03 | |
| ATOM | 1178 | O | LEU | | 317 | −8.963 | 102.082 | −66.655 | 1.00 | 44.87 | |
| ATOM | 1179 | N | ALA | | 318 | −10.067 | 102.305 | −64.710 | 1.00 | 43.87 | |
| ATOM | 1180 | CA | ALA | | 318 | −9.683 | 103.686 | −64.554 | 1.00 | 44.02 | |
| ATOM | 1181 | CB | ALA | | 318 | −10.475 | 104.343 | −63.434 | 1.00 | 44.38 | |
| ATOM | 1182 | C | ALA | | 318 | −8.210 | 103.769 | −64.246 | 1.00 | 43.01 | |
| ATOM | 1183 | O | ALA | | 318 | −7.665 | 102.963 | −63.497 | 1.00 | 44.82 | |
| ATOM | 1184 | N | PRO | | 319 | −7.541 | 104.758 | −64.815 | 1.00 | 42.59 | |
| ATOM | 1185 | CD | PRO | | 319 | −8.016 | 105.783 | −65.749 | 1.00 | 43.21 | |
| ATOM | 1186 | CA | PRO | | 319 | −6.114 | 104.908 | −64.555 | 1.00 | 42.84 | |
| ATOM | 1187 | CB | PRO | | 319 | −5.782 | 106.290 | −65.157 | 1.00 | 43.35 | |
| ATOM | 1188 | CG | PRO | | 319 | −7.112 | 106.935 | −65.379 | 1.00 | 45.27 | |
| ATOM | 1189 | C | PRO | | 319 | −5.721 | 104.805 | −63.091 | 1.00 | 43.49 | |
| ATOM | 1190 | O | PRO | | 319 | −4.710 | 104.187 | −62.774 | 1.00 | 44.53 | |
| ATOM | 1191 | N | GLU | | 320 | −6.493 | 105.393 | −62.182 | 1.00 | 43.03 | |
| ATOM | 1192 | CA | GLU | | 320 | −6.079 | 105.312 | −60.790 | 1.00 | 44.76 | |
| ATOM | 1193 | CB | GLU | | 320 | −6.808 | 106.355 | −59.893 | 1.00 | 48.10 | |
| ATOM | 1194 | CG | GLU | | 320 | −8.326 | 106.204 | −59.762 | 1.00 | 46.52 | |
| ATOM | 1195 | CD | GLU | | 320 | −9.071 | 106.821 | −60.926 | 1.00 | 48.46 | |
| ATOM | 1196 | OE1 | GLU | | 320 | −8.493 | 106.872 | −62.040 | 1.00 | 47.31 | |
| ATOM | 1197 | OE2 | GLU | | 320 | −10.237 | 107.235 | −60.723 | 1.00 | 47.04 | |
| ATOM | 1198 | C | GLU | | 320 | −6.263 | 103.894 | −60.262 | 1.00 | 44.72 | |
| ATOM | 1199 | O | GLU | | 320 | −5.746 | 103.530 | −59.215 | 1.00 | 43.21 | |
| ATOM | 1200 | N | VAL | | 321 | −7.016 | 103.081 | −60.975 | 1.00 | 46.54 | |
| ATOM | 1201 | CA | VAL | | 321 | −7.154 | 101.715 | −60.522 | 1.00 | 49.76 | |
| ATOM | 1202 | CB | VAL | | 321 | −8.401 | 101.062 | −61.087 | 1.00 | 50.16 | |
| ATOM | 1203 | CG1 | VAL | | 321 | −8.314 | 99.545 | −60.908 | 1.00 | 48.56 | |
| ATOM | 1204 | CG2 | VAL | | 321 | −9.627 | 101.626 | −60.374 | 1.00 | 50.78 | |
| ATOM | 1205 | C | VAL | | 321 | −5.904 | 100.949 | −60.980 | 1.00 | 51.61 | |
| ATOM | 1206 | O | VAL | | 321 | −5.328 | 100.190 | −60.199 | 1.00 | 53.12 | |
| ATOM | 1207 | N | LEU | | 322 | −5.492 | 101.168 | −62.235 | 1.00 | 50.45 | |
| ATOM | 1208 | CA | LEU | | 322 | −4.291 | 100.537 | −62.804 | 1.00 | 49.32 | |
| ATOM | 1209 | CB | LEU | | 322 | −4.119 | 100.915 | −64.272 | 1.00 | 47.27 | |
| ATOM | 1210 | CG | LEU | | 322 | −5.229 | 100.509 | −65.231 | 1.00 | 46.23 | |
| ATOM | 1211 | CD1 | LEU | | 322 | −4.944 | 101.014 | −66.650 | 1.00 | 46.57 | |
| ATOM | 1212 | CD2 | LEU | | 322 | −5.341 | 98.997 | −65.194 | 1.00 | 47.37 | |
| ATOM | 1213 | C | LEU | | 322 | −3.041 | 100.994 | −62.062 | 1.00 | 49.93 | |
| ATOM | 1214 | O | LEU | | 322 | −2.205 | 100.196 | −61.676 | 1.00 | 50.30 | |
| ATOM | 1215 | N | GLU | | 323 | −2.907 | 102.285 | −61.840 | 1.00 | 50.92 | |
| ATOM | 1216 | CA | GLU | | 323 | −1.712 | 102.736 | −61.160 | 1.00 | 53.42 | |
| ATOM | 1217 | CB | GLU | | 323 | −1.429 | 104.196 | −61.515 | 1.00 | 51.33 | |
| ATOM | 1218 | CG | GLU | | 323 | −0.210 | 104.753 | −60.819 | 1.00 | 52.71 | |
| ATOM | 1219 | CD | GLU | | 323 | 0.208 | 106.094 | −61.364 | 1.00 | 54.31 | |
| ATOM | 1220 | OE1 | GLU | | 323 | −0.658 | 106.825 | −61.899 | 1.00 | 53.42 | |
| ATOM | 1221 | OE2 | GLU | | 323 | 1.406 | 106.422 | −61.240 | 1.00 | 54.99 | |
| ATOM | 1222 | C | GLU | | 323 | −1.688 | 102.539 | −59.638 | 1.00 | 54.99 | |
| ATOM | 1223 | O | GLU | | 323 | −0.654 | 102.178 | −59.074 | 1.00 | 56.42 | |
| ATOM | 1224 | N | ASP | | 324 | −2.812 | 102.748 | −58.967 | 1.00 | 55.14 | |
| ATOM | 1225 | CA | ASP | | 324 | −2.823 | 102.608 | −57.530 | 1.00 | 54.80 | |
| ATOM | 1226 | CB | ASP | | 324 | −2.933 | 103.980 | −56.895 | 1.00 | 57.17 | |
| ATOM | 1227 | CG | ASP | | 324 | −1.637 | 104.719 | −56.952 | 1.00 | 60.24 | |
| ATOM | 1228 | OD1 | ASP | | 324 | −0.632 | 104.083 | −56.548 | 1.00 | 61.08 | |
| ATOM | 1229 | OD2 | ASP | | 324 | −1.620 | 105.898 | −57.395 | 1.00 | 58.44 | |
| ATOM | 1230 | C | ASP | | 324 | −3.854 | 101.701 | −56.917 | 1.00 | 54.98 | |
| ATOM | 1231 | O | ASP | | 324 | −3.970 | 101.658 | −55.696 | 1.00 | 54.52 | |
| ATOM | 1232 | N | ASN | | 325 | −4.612 | 100.984 | −57.734 | 1.00 | 55.35 | |
| ATOM | 1233 | CA | ASN | | 325 | −5.606 | 100.089 | −57.166 | 1.00 | 55.66 | |
| ATOM | 1234 | CB | ASN | | 325 | −4.882 | 99.039 | −56.340 | 1.00 | 55.33 | |
| ATOM | 1235 | CG | ASN | | 325 | −5.795 | 97.986 | −55.834 | 1.00 | 57.26 | |
| ATOM | 1236 | OD1 | ASN | | 325 | −6.543 | 97.380 | −56.608 | 1.00 | 56.07 | |
| ATOM | 1237 | ND2 | ASN | | 325 | −5.749 | 97.741 | −54.526 | 1.00 | 58.55 | |
| ATOM | 1238 | C | ASN | | 325 | −6.530 | 100.918 | −56.265 | 1.00 | 55.25 | |
| ATOM | 1239 | O | ASN | | 325 | −6.901 | 100.502 | −55.172 | 1.00 | 53.76 | |
| ATOM | 1240 | N | ASP | | 326 | −6.896 | 102.096 | −56.752 | 1.00 | 56.48 | |
| ATOM | 1241 | CA | ASP | | 326 | −7.726 | 103.040 | −56.007 | 1.00 | 57.01 | |
| ATOM | 1242 | CB | ASP | | 326 | −7.046 | 104.412 | −56.046 | 1.00 | 60.51 | |
| ATOM | 1243 | CG | ASP | | 326 | −7.879 | 105.494 | −55.403 | 1.00 | 64.02 | |
| ATOM | 1244 | OD1 | ASP | | 326 | −7.706 | 106.683 | −55.764 | 1.00 | 64.46 | |
| ATOM | 1245 | OD2 | ASP | | 326 | −8.699 | 105.150 | −54.529 | 1.00 | 66.73 | |
| ATOM | 1246 | C | ASP | | 326 | −9.165 | 103.162 | −56.525 | 1.00 | 54.60 | |
| ATOM | 1247 | O | ASP | | 326 | −9.460 | 103.977 | −57.398 | 1.00 | 54.57 | |
| ATOM | 1248 | N | TYR | | 327 | −10.057 | 102.355 | −55.969 | 1.00 | 51.99 | |
| ATOM | 1249 | CA | TYR | | 327 | −11.453 | 102.376 | −56.364 | 1.00 | 50.16 | |
| ATOM | 1250 | CB | TYR | | 327 | −12.096 | 101.007 | −56.168 | 1.00 | 49.27 | |
| ATOM | 1251 | CG | TYR | | 327 | −11.586 | 99.951 | −57.109 | 1.00 | 50.05 | |
| ATOM | 1252 | CD1 | TYR | | 327 | −10.366 | 99.315 | −56.879 | 1.00 | 48.99 | |
| ATOM | 1253 | CE1 | TYR | | 327 | −9.893 | 98.360 | −57.746 | 1.00 | 48.59 | |
| ATOM | 1254 | CD2 | TYR | | 327 | −12.313 | 99.597 | −58.240 | 1.00 | 48.52 | |
| ATOM | 1255 | CE2 | TYR | | 327 | −11.842 | 98.640 | −59.124 | 1.00 | 49.50 | |
| ATOM | 1256 | CZ | TYR | | 327 | −10.630 | 98.025 | −58.872 | 1.00 | 50.76 | |
| ATOM | 1257 | OH | TYR | | 327 | −10.140 | 97.087 | −59.766 | 1.00 | 51.99 | |
| ATOM | 1258 | C | TYR | | 327 | −12.272 | 103.408 | −55.598 | 1.00 | 49.40 | |
| ATOM | 1259 | O | TYR | | 327 | −13.166 | 103.053 | −54.833 | 1.00 | 50.18 | |
| ATOM | 1260 | N | GLY | | 328 | −11.956 | 104.679 | −55.813 | 1.00 | 48.90 | |
| ATOM | 1261 | CA | GLY | | 328 | −12.686 | 105.757 | −55.186 | 1.00 | 46.25 | |
| ATOM | 1262 | C | GLY | | 328 | −13.806 | 106.160 | −56.126 | 1.00 | 46.83 | |
| ATOM | 1263 | O | GLY | | 328 | −13.874 | 105.679 | −57.272 | 1.00 | 47.34 | |
| ATOM | 1264 | N | ARG | | 329 | −14.669 | 107.048 | −55.651 | 1.00 | 45.65 | |
| ATOM | 1265 | CA | ARG | | 329 | −15.816 | 107.525 | −56.407 | 1.00 | 44.38 | |
| ATOM | 1266 | CB | ARG | | 329 | −16.361 | 108.810 | −55.785 | 1.00 | 44.43 | |
| ATOM | 1267 | CG | ARG | | 329 | −16.333 | 108.891 | −54.284 | 1.00 | 49.59 | |
| ATOM | 1268 | CD | ARG | | 329 | −16.128 | 110.355 | −53.849 | 1.00 | 51.17 | |
| ATOM | 1269 | NE | ARG | | 329 | −16.659 | 111.214 | −54.890 | 1.00 | 51.31 | |
| ATOM | 1270 | CZ | ARG | | 329 | −16.190 | 112.410 | −55.212 | 1.00 | 52.20 | |
| ATOM | 1271 | NH1 | ARG | | 329 | −15.166 | 112.936 | −54.555 | 1.00 | 48.17 | |
| ATOM | 1272 | NH2 | ARG | | 329 | −16.720 | 113.044 | −56.257 | 1.00 | 53.25 | |
| ATOM | 1273 | C | ARG | | 329 | −15.542 | 107.822 | −57.872 | 1.00 | 42.12 | |
| ATOM | 1274 | O | ARG | | 329 | −16.340 | 107.523 | −58.743 | 1.00 | 43.69 | |
| ATOM | 1275 | N | ALA | | 330 | −14.426 | 108.462 | −58.144 | 1.00 | 42.72 | |
| ATOM | 1276 | CA | ALA | | 330 | −14.101 | 108.829 | −59.507 | 1.00 | 43.00 | |
| ATOM | 1277 | CB | ALA | | 330 | −12.729 | 109.445 | −59.541 | 1.00 | 43.84 | |
| ATOM | 1278 | C | ALA | | 330 | −14.192 | 107.714 | −60.542 | 1.00 | 42.15 | |
| ATOM | 1279 | O | ALA | | 330 | −14.655 | 107.948 | −61.654 | 1.00 | 44.24 | |
| ATOM | 1280 | N | VAL | | 331 | −13.765 | 106.507 | −60.197 | 1.00 | 40.09 | |
| ATOM | 1281 | CA | VAL | | 331 | −13.799 | 105.438 | −61.188 | 1.00 | 40.14 | |
| ATOM | 1282 | CB | VAL | | 331 | −13.295 | 104.083 | −60.599 | 1.00 | 39.98 | |
| ATOM | 1283 | CG1 | VAL | | 331 | −11.942 | 104.288 | −59.892 | 1.00 | 36.42 | |
| ATOM | 1284 | CG2 | VAL | | 331 | −14.339 | 103.500 | −59.661 | 1.00 | 37.20 | |
| ATOM | 1285 | C | VAL | | 331 | −15.170 | 105.233 | −61.816 | 1.00 | 40.34 | |
| ATOM | 1286 | O | VAL | | 331 | −15.285 | 104.727 | −62.937 | 1.00 | 43.22 | |
| ATOM | 1287 | N | ASP | | 332 | −16.219 | 105.622 | −61.104 | 1.00 | 38.59 | |
| ATOM | 1288 | CA | ASP | | 332 | −17.553 | 105.469 | −61.648 | 1.00 | 35.94 | |
| ATOM | 1289 | CB | ASP | | 332 | −18.621 | 105.649 | −60.545 | 1.00 | 37.31 | |
| ATOM | 1290 | CG | ASP | | 332 | −18.774 | 104.413 | −59.649 | 1.00 | 39.08 | |
| ATOM | 1291 | OD1 | ASP | | 332 | −18.465 | 103.304 | −60.134 | 1.00 | 39.92 | |
| ATOM | 1292 | OD2 | ASP | | 332 | −19.223 | 104.535 | −58.477 | 1.00 | 38.85 | |
| ATOM | 1293 | C | ASP | | 332 | −17.770 | 106.473 | −62.757 | 1.00 | 30.82 | |
| ATOM | 1294 | O | ASP | | 332 | −18.439 | 106.181 | −63.748 | 1.00 | 31.25 | |
| ATOM | 1295 | N | TRP | | 333 | −17.223 | 107.668 | −62.587 | 1.00 | 31.26 | |
| ATOM | 1296 | CA | TRP | | 333 | −17.379 | 108.720 | −63.594 | 1.00 | 33.91 | |
| ATOM | 1297 | CB | TRP | | 333 | −16.964 | 110.096 | −63.015 | 1.00 | 35.02 | |
| ATOM | 1298 | CG | TRP | | 333 | −17.919 | 110.550 | −61.926 | 1.00 | 32.79 | |
| ATOM | 1299 | CD2 | TRP | | 333 | −19.351 | 110.529 | −61.991 | 1.00 | 30.04 | |
| ATOM | 1300 | CE2 | TRP | | 333 | −19.830 | 110.827 | −60.706 | 1.00 | 31.33 | |
| ATOM | 1301 | CE3 | TRP | | 333 | −20.276 | 110.268 | −63.013 | 1.00 | 28.23 | |
| ATOM | 1302 | CD1 | TRP | | 333 | −17.601 | 110.871 | −60.647 | 1.00 | 33.52 | |
| ATOM | 1303 | NE1 | TRP | | 333 | −18.739 | 111.038 | −59.904 | 1.00 | 34.35 | |
| ATOM | 1304 | CZ2 | TRP | | 333 | −21.202 | 110.864 | −60.402 | 1.00 | 27.84 | |
| ATOM | 1305 | CZ3 | TRP | | 333 | −21.635 | 110.306 | −62.709 | 1.00 | 28.48 | |
| ATOM | 1306 | CH2 | TRP | | 333 | −22.080 | 110.599 | −61.408 | 1.00 | 23.78 | |
| ATOM | 1307 | C | TRP | | 333 | −16.542 | 108.336 | −64.808 | 1.00 | 35.02 | |
| ATOM | 1308 | O | TRP | | 333 | −16.960 | 108.555 | −65.942 | 1.00 | 35.03 | |
| ATOM | 1309 | N | TRP | | 334 | −15.378 | 107.731 | −64.562 | 1.00 | 36.30 | |
| ATOM | 1310 | CA | TRP | | 334 | −14.524 | 107.258 | −65.646 | 1.00 | 35.62 | |
| ATOM | 1311 | CB | TRP | | 334 | −13.216 | 106.715 | −65.052 | 1.00 | 37.90 | |
| ATOM | 1312 | CG | TRP | | 334 | −12.411 | 105.806 | −65.990 | 1.00 | 43.98 | |
| ATOM | 1313 | CD2 | TRP | | 334 | −11.457 | 106.215 | −66.977 | 1.00 | 43.83 | |
| ATOM | 1314 | CE2 | TRP | | 334 | −11.017 | 105.048 | −67.644 | 1.00 | 44.87 | |
| ATOM | 1315 | CE3 | TRP | | 334 | −10.930 | 107.454 | −67.365 | 1.00 | 45.29 | |
| ATOM | 1316 | CD1 | TRP | | 334 | −12.504 | 104.447 | −66.094 | 1.00 | 42.68 | |
| ATOM | 1317 | NE1 | TRP | | 334 | −11.672 | 103.987 | −67.083 | 1.00 | 44.91 | |
| ATOM | 1318 | CZ2 | TRP | | 334 | −10.081 | 105.080 | −68.683 | 1.00 | 42.82 | |
| ATOM | 1319 | CZ3 | TRP | | 334 | −9.984 | 107.487 | −68.416 | 1.00 | 45.27 | |
| ATOM | 1320 | CH2 | TRP | | 334 | −9.579 | 106.306 | −69.054 | 1.00 | 42.67 | |
| ATOM | 1321 | C | TRP | | 334 | −15.326 | 106.183 | −66.396 | 1.00 | 34.52 | |
| ATOM | 1322 | O | TRP | | 334 | −15.389 | 106.158 | −67.631 | 1.00 | 37.86 | |
| ATOM | 1323 | N | GLY | | 335 | −15.995 | 105.316 | −65.655 | 1.00 | 31.85 | |
| ATOM | 1324 | CA | GLY | | 335 | −16.782 | 104.301 | −66.311 | 1.00 | 30.60 | |
| ATOM | 1325 | C | GLY | | 335 | −17.915 | 104.888 | −67.118 | 1.00 | 33.86 | |
| ATOM | 1326 | O | GLY | | 335 | −18.303 | 104.347 | −68.171 | 1.00 | 34.29 | |
| ATOM | 1327 | N | LEU | | 336 | −18.482 | 105.984 | −66.618 | 1.00 | 34.61 | |
| ATOM | 1328 | CA | LEU | | 336 | −19.583 | 106.641 | −67.321 | 1.00 | 34.46 | |
| ATOM | 1329 | CB | LEU | | 336 | −20.166 | 107.826 | −66.504 | 1.00 | 31.10 | |
| ATOM | 1330 | CG | LEU | | 336 | −21.191 | 108.610 | −67.333 | 1.00 | 30.58 | |
| ATOM | 1331 | CD1 | LEU | | 336 | −22.347 | 107.686 | −67.703 | 1.00 | 28.91 | |
| ATOM | 1332 | CD2 | LEU | | 336 | −21.704 | 109.809 | −66.564 | 1.00 | 31.32 | |
| ATOM | 1333 | C | LEU | | 336 | −19.059 | 107.163 | −68.657 | 1.00 | 33.73 | |
| ATOM | 1334 | O | LEU | | 336 | −19.701 | 106.989 | −69.699 | 1.00 | 33.15 | |
| ATOM | 1335 | N | GLY | | 337 | −17.896 | 107.805 | −68.618 | 1.00 | 32.09 | |
| ATOM | 1336 | CA | GLY | | 337 | −17.323 | 108.318 | −69.849 | 1.00 | 35.61 | |
| ATOM | 1337 | C | GLY | | 337 | −17.013 | 107.276 | −70.923 | 1.00 | 36.20 | |
| ATOM | 1338 | O | GLY | | 337 | −17.314 | 107.484 | −72.109 | 1.00 | 35.81 | |
| ATOM | 1339 | N | VAL | | 338 | −16.406 | 106.162 | −70.518 | 1.00 | 35.93 | |
| ATOM | 1340 | CA | VAL | | 338 | −16.096 | 105.084 | −71.457 | 1.00 | 34.99 | |
| ATOM | 1341 | CB | VAL | | 338 | −15.373 | 103.917 | −70.744 | 1.00 | 35.15 | |
| ATOM | 1342 | CG1 | VAL | | 338 | −15.287 | 102.716 | −71.696 | 1.00 | 35.32 | |
| ATOM | 1343 | CG2 | VAL | | 338 | −14.010 | 104.370 | −70.249 | 1.00 | 29.80 | |
| ATOM | 1344 | C | VAL | | 338 | −17.391 | 104.542 | −72.064 | 1.00 | 36.10 | |
| ATOM | 1345 | O | VAL | | 338 | −17.439 | 104.198 | −73.221 | 1.00 | 35.73 | |
| ATOM | 1346 | N | VAL | | 339 | −18.448 | 104.475 | −71.269 | 1.00 | 37.38 | |
| ATOM | 1347 | CA | VAL | | 339 | −19.725 | 103.983 | −71.761 | 1.00 | 38.24 | |
| ATOM | 1348 | CB | VAL | | 339 | −20.707 | 103.767 | −70.600 | 1.00 | 38.51 | |
| ATOM | 1349 | CG1 | VAL | | 339 | −22.118 | 103.527 | −71.141 | 1.00 | 39.32 | |
| ATOM | 1350 | CG2 | VAL | | 339 | −20.242 | 102.629 | −69.746 | 1.00 | 36.32 | |
| ATOM | 1351 | C | VAL | | 339 | −20.389 | 104.935 | −72.769 | 1.00 | 41.68 | |
| ATOM | 1352 | O | VAL | | 339 | −20.982 | 104.496 | −73.753 | 1.00 | 42.94 | |
| ATOM | 1353 | N | MET | | 340 | −20.337 | 106.235 | −72.526 | 1.00 | 43.29 | |
| ATOM | 1354 | CA | MET | | 340 | −20.954 | 107.139 | −73.488 | 1.00 | 47.13 | |
| ATOM | 1355 | CB | MET | | 340 | −21.355 | 108.429 | −72.808 | 1.00 | 47.27 | |
| ATOM | 1356 | CG | MET | | 340 | −22.494 | 108.158 | −71.888 | 1.00 | 50.00 | |
| ATOM | 1357 | SD | MET | | 340 | −22.930 | 109.585 | −70.972 | 1.00 | 58.78 | |
| ATOM | 1358 | CE | MET | | 340 | −21.327 | 110.067 | −70.266 | 1.00 | 57.23 | |
| ATOM | 1359 | C | MET | | 340 | −20.068 | 107.395 | −74.697 | 1.00 | 47.39 | |
| ATOM | 1360 | O | MET | | 340 | −20.570 | 107.714 | −75.776 | 1.00 | 48.38 | |
| ATOM | 1361 | N | TYR | | 341 | −18.756 | 107.261 | −74.518 | 1.00 | 47.83 | |
| ATOM | 1362 | CA | TYR | | 341 | −17.820 | 107.397 | −75.638 | 1.00 | 47.94 | |
| ATOM | 1363 | CB | TYR | | 341 | −16.381 | 107.168 | −75.175 | 1.00 | 48.36 | |
| ATOM | 1364 | CG | TYR | | 341 | −15.347 | 107.294 | −76.274 | 1.00 | 50.05 | |
| ATOM | 1365 | CD1 | TYR | | 341 | −15.342 | 106.428 | −77.376 | 1.00 | 50.85 | |
| ATOM | 1366 | CE1 | TYR | | 341 | −14.383 | 106.550 | −78.390 | 1.00 | 49.93 | |
| ATOM | 1367 | CD2 | TYR | | 341 | −14.364 | 108.277 | −76.209 | 1.00 | 50.28 | |
| ATOM | 1368 | CE2 | TYR | | 341 | −13.400 | 108.407 | −77.201 | 1.00 | 49.75 | |
| ATOM | 1369 | CZ | TYR | | 341 | −13.418 | 107.547 | −78.289 | 1.00 | 50.14 | |
| ATOM | 1370 | OH | TYR | | 341 | −12.481 | 107.722 | −79.273 | 1.00 | 49.73 | |
| ATOM | 1371 | C | TYR | | 341 | −18.185 | 106.302 | −76.645 | 1.00 | 47.92 | |
| ATOM | 1372 | O | TYR | | 341 | −18.215 | 106.543 | −77.850 | 1.00 | 47.96 | |
| ATOM | 1373 | N | GLU | | 342 | −18.501 | 105.104 | −76.155 | 1.00 | 46.58 | |
| ATOM | 1374 | CA | GLU | | 342 | −18.839 | 104.033 | −77.068 | 1.00 | 46.00 | |
| ATOM | 1375 | CB | GLU | | 342 | −18.758 | 102.660 | −76.376 | 1.00 | 47.87 | |
| ATOM | 1376 | CG | GLU | | 342 | −18.397 | 101.539 | −77.352 | 1.00 | 48.85 | |
| ATOM | 1377 | CD | GLU | | 342 | −18.527 | 100.096 | −76.802 | 1.00 | 51.28 | |
| ATOM | 1378 | OE1 | GLU | | 342 | −18.160 | 99.817 | −75.632 | 1.00 | 49.65 | |
| ATOM | 1379 | OE2 | GLU | | 342 | −18.984 | 99.223 | −77.578 | 1.00 | 50.90 | |
| ATOM | 1380 | C | GLU | | 342 | −20.210 | 104.220 | −77.677 | 1.00 | 45.28 | |
| ATOM | 1381 | O | GLU | | 342 | −20.423 | 103.854 | −78.828 | 1.00 | 46.44 | |
| ATOM | 1382 | N | MET | | 343 | −21.151 | 104.768 | −76.915 | 1.00 | 45.59 | |
| ATOM | 1383 | CA | MET | | 343 | −22.521 | 104.990 | −77.419 | 1.00 | 45.57 | |
| ATOM | 1384 | CB | MET | | 343 | −23.434 | 105.507 | −76.317 | 1.00 | 45.82 | |
| ATOM | 1385 | CG | MET | | 343 | −23.647 | 104.552 | −75.212 | 1.00 | 46.42 | |
| ATOM | 1386 | SD | MET | | 343 | −24.664 | 105.261 | −73.950 | 1.00 | 42.59 | |
| ATOM | 1387 | CE | MET | | 343 | −26.008 | 104.169 | −74.111 | 1.00 | 42.20 | |
| ATOM | 1388 | C | MET | | 343 | −22.575 | 106.009 | −78.527 | 1.00 | 46.54 | |
| ATOM | 1389 | O | MET | | 343 | −23.347 | 105.874 | −79.470 | 1.00 | 46.11 | |
| ATOM | 1390 | N | MET | | 344 | −21.766 | 107.048 | −78.379 | 1.00 | 48.19 | |
| ATOM | 1391 | CA | MET | | 344 | −21.725 | 108.124 | −79.337 | 1.00 | 52.87 | |
| ATOM | 1392 | CB | MET | | 344 | −21.468 | 109.441 | −78.583 | 1.00 | 54.70 | |
| ATOM | 1393 | CG | MET | | 344 | −22.732 | 109.950 | −77.819 | 1.00 | 53.79 | |
| ATOM | 1394 | SD | MET | | 344 | −22.469 | 111.313 | −76.629 | 1.00 | 53.65 | |
| ATOM | 1395 | CE | MET | | 344 | −20.927 | 111.957 | −77.209 | 1.00 | 55.26 | |
| ATOM | 1396 | C | MET | | 344 | −20.741 | 107.922 | −80.496 | 1.00 | 55.14 | |
| ATOM | 1397 | O | MET | | 344 | −20.982 | 108.421 | −81.585 | 1.00 | 56.82 | |
| ATOM | 1398 | C | CYS | | 345 | −19.659 | 107.172 | −80.278 | 1.00 | 56.22 | |
| ATOM | 1399 | CA | CYS | | 345 | −18.664 | 106.907 | −81.327 | 1.00 | 54.43 | |
| ATOM | 1400 | CB | CYS | | 345 | −17.266 | 107.057 | −80.757 | 1.00 | 53.88 | |
| ATOM | 1401 | SG | CYS | | 345 | −17.096 | 108.616 | −79.854 | 1.00 | 55.65 | |
| ATOM | 1402 | C | CYS | | 345 | −18.832 | 105.520 | −81.967 | 1.00 | 53.79 | |
| ATOM | 1403 | O | CYS | | 345 | −18.521 | 105.334 | −83.139 | 1.00 | 54.14 | |
| ATOM | 1404 | N | GLY | | 346 | −19.345 | 104.561 | −81.202 | 1.00 | 53.60 | |
| ATOM | 1405 | CA | GLY | | 346 | −19.583 | 103.221 | −81.724 | 1.00 | 53.07 | |
| ATOM | 1406 | C | GLY | | 346 | −18.442 | 102.236 | −81.509 | 1.00 | 54.24 | |
| ATOM | 1407 | O | GLY | | 346 | −18.529 | 101.072 | −81.904 | 1.00 | 54.77 | |
| ATOM | 1408 | N | ARG | | 347 | −17.385 | 102.695 | −80.856 | 1.00 | 52.18 | |
| ATOM | 1409 | CA | ARG | | 347 | −16.216 | 101.882 | −80.625 | 1.00 | 52.52 | |
| ATOM | 1410 | CB | ARG | | 347 | −15.282 | 102.036 | −81.825 | 1.00 | 53.58 | |
| ATOM | 1411 | CG | ARG | | 347 | −14.898 | 103.488 | −82.028 | 1.00 | 56.50 | |
| ATOM | 1412 | CD | ARG | | 347 | −13.855 | 103.693 | −83.108 | 1.00 | 60.38 | |
| ATOM | 1413 | NE | ARG | | 347 | −13.384 | 105.084 | −83.188 | 1.00 | 62.75 | |
| ATOM | 1414 | CZ | ARG | | 347 | −14.052 | 106.088 | −83.761 | 1.00 | 63.87 | |
| ATOM | 1415 | NH1 | ARG | | 347 | −15.241 | 105.875 | −84.321 | 1.00 | 62.78 | |
| ATOM | 1416 | NH2 | ARG | | 347 | −13.525 | 107.312 | −83.780 | 1.00 | 64.47 | |
| ATOM | 1417 | C | ARG | | 347 | −15.537 | 102.435 | −79.380 | 1.00 | 51.29 | |
| ATOM | 1418 | O | ARG | | 347 | −15.779 | 103.588 | −79.017 | 1.00 | 52.32 | |
| ATOM | 1419 | N | LEU | | 348 | −14.688 | 101.628 | −78.738 | 1.00 | 48.98 | |
| ATOM | 1420 | CA | LEU | | 348 | −13.954 | 102.064 | −77.550 | 1.00 | 46.80 | |
| ATOM | 1421 | CB | LEU | | 348 | −13.391 | 100.855 | −76.797 | 1.00 | 42.70 | |
| ATOM | 1422 | CG | LEU | | 348 | −14.472 | 100.098 | −76.001 | 1.00 | 41.56 | |
| ATOM | 1423 | CD1 | LEU | | 348 | −14.061 | 98.674 | −75.719 | 1.00 | 40.62 | |
| ATOM | 1424 | CD2 | LEU | | 348 | −14.727 | 100.812 | −74.690 | 1.00 | 41.06 | |
| ATOM | 1425 | C | LEU | | 348 | −12.857 | 103.047 | −77.964 | 1.00 | 47.61 | |
| ATOM | 1426 | O | LEU | | 348 | −12.566 | 103.186 | −79.139 | 1.00 | 51.11 | |
| ATOM | 1427 | N | PRO | | 349 | −12.245 | 103.757 | −77.013 | 1.00 | 47.51 | |
| ATOM | 1428 | CD | PRO | | 349 | −12.581 | 103.882 | −75.592 | 1.00 | 46.67 | |
| ATOM | 1429 | CA | PRO | | 349 | −11.205 | 104.716 | −77.385 | 1.00 | 47.57 | |
| ATOM | 1430 | CB | PRO | | 349 | −10.901 | 105.436 | −76.075 | 1.00 | 47.25 | |
| ATOM | 1431 | CG | PRO | | 349 | −11.319 | 104.493 | −75.056 | 1.00 | 47.82 | |
| ATOM | 1432 | C | PRO | | 349 | −9.953 | 104.221 | −78.060 | 1.00 | 51.21 | |
| ATOM | 1433 | O | PRO | | 349 | −9.329 | 104.949 | −78.826 | 1.00 | 50.75 | |
| ATOM | 1434 | N | PHE | | 350 | −9.557 | 102.996 | −77.770 | 1.00 | 53.02 | |
| ATOM | 1435 | CA | PHE | | 350 | −8.355 | 102.466 | −78.390 | 1.00 | 54.38 | |
| ATOM | 1436 | CB | PHE | | 350 | −7.325 | 102.172 | −77.304 | 1.00 | 50.15 | |
| ATOM | 1437 | CG | PHE | | 350 | −7.020 | 103.350 | −76.425 | 1.00 | 46.03 | |
| ATOM | 1438 | CD1 | PHE | | 350 | −7.210 | 103.274 | −75.047 | 1.00 | 43.07 | |
| ATOM | 1439 | CD2 | PHE | | 350 | −6.464 | 104.505 | −76.960 | 1.00 | 43.87 | |
| ATOM | 1440 | CE1 | PHE | | 350 | −6.845 | 104.319 | −74.210 | 1.00 | 38.42 | |
| ATOM | 1441 | CE2 | PHE | | 350 | −6.095 | 105.553 | −76.140 | 1.00 | 38.64 | |
| ATOM | 1442 | CZ | PHE | | 350 | −6.282 | 105.460 | −74.754 | 1.00 | 41.34 | |
| ATOM | 1443 | C | PHE | | 350 | −8.726 | 101.197 | −79.144 | 1.00 | 57.61 | |
| ATOM | 1444 | O | PHE | | 350 | −7.941 | 100.259 | −79.232 | 1.00 | 57.75 | |
| ATOM | 1445 | N | TYR | | 351 | −9.934 | 101.191 | −79.697 | 1.00 | 62.81 | |
| ATOM | 1446 | CA | TYR | | 351 | −10.468 | 100.028 | −80.396 | 1.00 | 67.96 | |
| ATOM | 1447 | CB | TYR | | 351 | −11.808 | 100.368 | −81.052 | 1.00 | 69.37 | |
| ATOM | 1448 | CG | TYR | | 351 | −11.685 | 100.990 | −82.423 | 1.00 | 72.39 | |
| ATOM | 1449 | CD1 | TYR | | 351 | −12.414 | 100.479 | −83.504 | 1.00 | 73.37 | |
| ATOM | 1450 | CE1 | TYR | | 351 | −12.300 | 101.034 | −84.777 | 1.00 | 74.61 | |
| ATOM | 1451 | CD2 | TYR | | 351 | −10.836 | 102.081 | −82.650 | 1.00 | 72.88 | |
| ATOM | 1452 | CE2 | TYR | | 351 | −10.717 | 102.647 | −83.922 | 1.00 | 73.95 | |
| ATOM | 1453 | CZ | TYR | | 351 | −11.454 | 102.118 | −84.979 | 1.00 | 74.79 | |
| ATOM | 1454 | OH | TYR | | 351 | −11.372 | 102.676 | −86.238 | 1.00 | 76.57 | |
| ATOM | 1455 | C | TYR | | 351 | −9.544 | 99.404 | −81.433 | 1.00 | 70.44 | |
| ATOM | 1456 | O | TYR | | 351 | −9.595 | 98.196 | −81.655 | 1.00 | 71.41 | |
| ATOM | 1457 | N | ASN | | 352 | −8.718 | 100.208 | −82.087 | 1.00 | 72.22 | |
| ATOM | 1458 | CA | ASN | | 352 | −7.815 | 99.638 | −83.071 | 1.00 | 76.18 | |
| ATOM | 1459 | CB | ASN | | 352 | −7.629 | 100.581 | −84.268 | 1.00 | 77.45 | |
| ATOM | 1460 | CG | ASN | | 352 | −7.433 | 102.021 | −83.857 | 1.00 | 79.51 | |
| ATOM | 1461 | OD1 | ASN | | 352 | −7.062 | 102.863 | −84.678 | 1.00 | 80.45 | |
| ATOM | 1462 | ND2 | ASN | | 352 | −7.687 | 102.320 | −82.585 | 1.00 | 80.65 | |
| ATOM | 1463 | C | ASN | | 352 | −6.466 | 99.289 | −82.449 | 1.00 | 77.72 | |
| ATOM | 1464 | O | ASN | | 352 | −6.016 | 98.146 | −82.564 | 1.00 | 79.04 | |
| ATOM | 1465 | N | GLN | | 353 | −5.837 | 100.251 | −81.773 | 1.00 | 78.39 | |
| ATOM | 1466 | CA | GLN | | 353 | −4.542 | 100.018 | −81.150 | 1.00 | 79.59 | |
| ATOM | 1467 | CB | GLN | | 353 | −4.189 | 101.156 | −80.209 | 1.00 | 79.95 | |
| ATOM | 1468 | CG | GLN | | 353 | −3.895 | 102.458 | −80.902 | 1.00 | 82.10 | |
| ATOM | 1469 | CD | GLN | | 353 | −2.971 | 103.349 | −80.082 | 1.00 | 84.82 | |
| ATOM | 1470 | OE1 | GLN | | 353 | −1.763 | 103.100 | −79.991 | 1.00 | 84.86 | |
| ATOM | 1471 | NE2 | GLN | | 353 | −3.537 | 104.389 | −79.470 | 1.00 | 85.81 | |
| ATOM | 1472 | C | GLN | | 353 | −4.460 | 98.699 | −80.395 | 1.00 | 81.13 | |
| ATOM | 1473 | O | GLN | | 353 | −4.997 | 98.564 | −79.301 | 1.00 | 82.46 | |
| ATOM | 1474 | N | ASP | | 354 | −3.764 | 97.740 | −80.996 | 1.00 | 82.13 | |
| ATOM | 1475 | CA | ASP | | 354 | −3.569 | 96.402 | −80.449 | 1.00 | 83.41 | |
| ATOM | 1476 | CB | ASP | | 354 | −2.105 | 96.210 | −80.042 | 1.00 | 85.22 | |
| ATOM | 1477 | CG | ASP | | 354 | −1.671 | 94.748 | −80.088 | 1.00 | 86.43 | |
| ATOM | 1478 | OD1 | ASP | | 354 | −2.390 | 93.884 | −79.525 | 1.00 | 87.08 | |
| ATOM | 1479 | OD2 | ASP | | 354 | −0.606 | 94.467 | −80.686 | 1.00 | 86.75 | |
| ATOM | 1480 | C | ASP | | 354 | −4.483 | 96.079 | −79.273 | 1.00 | 83.06 | |
| ATOM | 1481 | O | ASP | | 354 | −5.701 | 96.095 | −79.417 | 1.00 | 83.88 | |
| ATOM | 1482 | N | HIS | | 355 | −3.905 | 95.786 | −78.112 | 1.00 | 82.18 | |
| ATOM | 1483 | CA | HIS | | 355 | −4.711 | 95.460 | −76.950 | 1.00 | 81.42 | |
| ATOM | 1484 | CB | HIS | | 355 | −5.566 | 94.239 | −77.230 | 1.00 | 83.37 | |
| ATOM | 1485 | CG | HIS | | 355 | −6.197 | 93.675 | −76.003 | 1.00 | 86.62 | |
| ATOM | 1486 | CD2 | HIS | | 355 | −6.418 | 92.398 | −75.613 | 1.00 | 88.45 | |
| ATOM | 1487 | ND1 | HIS | | 355 | −6.672 | 94.474 | −74.985 | 1.00 | 88.33 | |
| ATOM | 1488 | CE1 | HIS | | 355 | −7.155 | 93.714 | −74.019 | 1.00 | 89.36 | |
| ATOM | 1489 | NE2 | HIS | | 355 | −7.014 | 92.449 | −74.375 | 1.00 | 89.98 | |
| ATOM | 1490 | C | HIS | | 355 | −3.936 | 95.230 | −75.662 | 1.00 | 80.28 | |
| ATOM | 1491 | O | HIS | | 355 | −4.452 | 95.461 | −74.570 | 1.00 | 79.78 | |
| ATOM | 1492 | N | GLU | | 356 | −2.710 | 94.741 | −75.774 | 1.00 | 78.69 | |
| ATOM | 1493 | CA | GLU | | 356 | −1.894 | 94.533 | −74.589 | 1.00 | 77.64 | |
| ATOM | 1494 | CB | GLU | | 356 | −0.815 | 93.496 | −74.868 | 1.00 | 81.61 | |
| ATOM | 1495 | CG | GLU | | 356 | −1.343 | 92.213 | −75.448 | 1.00 | 84.72 | |
| ATOM | 1496 | CD | GLU | | 356 | −0.260 | 91.445 | −76.163 | 1.00 | 87.70 | |
| ATOM | 1497 | OE1 | GLU | | 356 | 0.395 | 92.041 | −77.054 | 1.00 | 89.08 | |
| ATOM | 1498 | OE2 | GLU | | 356 | −0.061 | 90.253 | −75.840 | 1.00 | 89.45 | |
| ATOM | 1499 | C | GLU | | 356 | −1.251 | 95.882 | −74.289 | 1.00 | 75.04 | |
| ATOM | 1500 | O | GLU | | 356 | −0.439 | 96.019 | −73.374 | 1.00 | 73.95 | |
| ATOM | 1501 | N | ARG | | 357 | −1.633 | 96.869 | −75.095 | 1.00 | 72.08 | |
| ATOM | 1502 | CA | ARG | | 357 | −1.153 | 98.231 | −74.972 | 1.00 | 69.60 | |
| ATOM | 1503 | CB | ARG | | 357 | −1.078 | 98.867 | −76.353 | 1.00 | 71.43 | |
| ATOM | 1504 | CG | ARG | | 357 | 0.094 | 98.346 | −77.125 | 1.00 | 76.13 | |
| ATOM | 1505 | CD | ARG | | 357 | 0.055 | 98.637 | −78.604 | 1.00 | 78.63 | |
| ATOM | 1506 | NE | ARG | | 357 | 1.141 | 97.902 | −79.249 | 1.00 | 80.96 | |
| ATOM | 1507 | CZ | ARG | | 357 | 1.347 | 97.865 | −80.557 | 1.00 | 82.63 | |
| ATOM | 1508 | NH1 | ARG | | 357 | 0.533 | 98.534 | −81.368 | 1.00 | 82.83 | |
| ATOM | 1509 | NH2 | ARG | | 357 | 2.354 | 97.148 | −81.049 | 1.00 | 82.20 | |
| ATOM | 1510 | C | ARG | | 357 | −2.058 | 99.052 | −74.078 | 1.00 | 66.85 | |
| ATOM | 1511 | O | ARG | | 357 | −1.690 | 100.143 | −73.632 | 1.00 | 66.26 | |
| ATOM | 1512 | N | LEU | | 358 | −3.240 | 98.510 | −73.806 | 1.00 | 63.02 | |
| ATOM | 1513 | CA | LEU | | 358 | −4.222 | 99.197 | −72.983 | 1.00 | 59.37 | |
| ATOM | 1514 | CB | LEU | | 358 | −5.409 | 98.291 | −72.688 | 1.00 | 58.41 | |
| ATOM | 1515 | CG | LEU | | 358 | −6.414 | 98.354 | −73.833 | 1.00 | 57.85 | |
| ATOM | 1516 | CD1 | LEU | | 358 | −7.578 | 97.420 | −73.549 | 1.00 | 56.87 | |
| ATOM | 1517 | CD2 | LEU | | 358 | −6.885 | 99.795 | −74.004 | 1.00 | 55.74 | |
| ATOM | 1518 | C | LEU | | 358 | −3.682 | 99.746 | −71.698 | 1.00 | 57.04 | |
| ATOM | 1519 | O | LEU | | 358 | −4.055 | 100.839 | −71.308 | 1.00 | 55.89 | |
| ATOM | 1520 | N | PHE | | 359 | −2.798 | 99.005 | −71.043 | 1.00 | 55.87 | |
| ATOM | 1521 | CA | PHE | | 359 | −2.240 | 99.479 | −69.785 | 1.00 | 55.06 | |
| ATOM | 1522 | CB | PHE | | 359 | −1.221 | 98.495 | −69.214 | 1.00 | 51.89 | |
| ATOM | 1523 | CG | PHE | | 359 | −0.671 | 98.915 | −67.881 | 1.00 | 51.13 | |
| ATOM | 1524 | CD1 | PHE | | 359 | −1.391 | 98.693 | −66.715 | 1.00 | 48.78 | |
| ATOM | 1525 | CD2 | PHE | | 359 | 0.553 | 99.567 | −67.797 | 1.00 | 51.18 | |
| ATOM | 1526 | CE1 | PHE | | 359 | −0.902 | 99.114 | −65.483 | 1.00 | 50.68 | |
| ATOM | 1527 | CE2 | PHE | | 359 | 1.055 | 99.993 | −66.572 | 1.00 | 50.67 | |
| ATOM | 1528 | CZ | PHE | | 359 | 0.327 | 99.769 | −65.409 | 1.00 | 51.77 | |
| ATOM | 1529 | C | PHE | | 359 | −1.578 | 100.842 | −69.959 | 1.00 | 55.16 | |
| ATOM | 1530 | O | PHE | | 359 | −1.881 | 101.776 | −69.205 | 1.00 | 57.22 | |
| ATOM | 1531 | N | GLU | | 360 | −0.696 | 100.963 | −70.951 | 1.00 | 53.44 | |
| ATOM | 1532 | CA | GLU | | 360 | −0.001 | 102.229 | −71.182 | 1.00 | 53.14 | |
| ATOM | 1533 | CB | GLU | | 360 | 1.252 | 102.050 | −72.046 | 1.00 | 54.80 | |
| ATOM | 1534 | CG | GLU | | 360 | 2.070 | 100.799 | −71.810 | 1.00 | 58.70 | |
| ATOM | 1535 | CD | GLU | | 360 | 1.473 | 99.577 | −72.494 | 1.00 | 58.32 | |
| ATOM | 1536 | OE1 | GLU | | 360 | 0.856 | 98.753 | −71.790 | 1.00 | 57.65 | |
| ATOM | 1537 | OE2 | GLU | | 360 | 1.617 | 99.451 | −73.733 | 1.00 | 58.10 | |
| ATOM | 1538 | C | GLU | | 360 | −0.872 | 103.280 | −71.863 | 1.00 | 51.75 | |
| ATOM | 1539 | O | GLU | | 360 | −0.661 | 104.478 | −71.680 | 1.00 | 50.13 | |
| ATOM | 1540 | N | LEU | | 361 | −1.825 | 102.835 | −72.673 | 1.00 | 50.27 | |
| ATOM | 1541 | CA | LEU | | 361 | −2.686 | 103.771 | −73.365 | 1.00 | 50.68 | |
| ATOM | 1542 | CB | LEU | | 361 | −3.478 | 103.054 | −74.461 | 1.00 | 50.12 | |
| ATOM | 1543 | CG | LEU | | 361 | −2.623 | 102.536 | −75.628 | 1.00 | 51.08 | |
| ATOM | 1544 | CD1 | LEU | | 361 | −3.549 | 101.911 | −76.651 | 1.00 | 49.83 | |
| ATOM | 1545 | CD2 | LEU | | 361 | −1.803 | 103.687 | −76.275 | 1.00 | 48.70 | |
| ATOM | 1546 | C | LEU | | 361 | −3.622 | 104.481 | −72.379 | 1.00 | 50.93 | |
| ATOM | 1547 | O | LEU | | 361 | −3.717 | 105.709 | −72.374 | 1.00 | 52.21 | |
| ATOM | 1548 | N | ILE | | 362 | −4.293 | 103.706 | −71.537 | 1.00 | 49.07 | |
| ATOM | 1549 | CA | ILE | | 362 | −5.197 | 104.262 | −70.551 | 1.00 | 47.02 | |
| ATOM | 1550 | CB | ILE | | 362 | −5.867 | 103.135 | −69.731 | 1.00 | 45.45 | |
| ATOM | 1551 | CG2 | ILE | | 362 | −6.513 | 103.702 | −68.460 | 1.00 | 42.01 | |
| ATOM | 1552 | CG1 | ILE | | 362 | −6.873 | 102.400 | −70.623 | 1.00 | 41.50 | |
| ATOM | 1553 | CD1 | ILE | | 362 | −7.453 | 101.158 | −69.981 | 1.00 | 41.44 | |
| ATOM | 1554 | C | ILE | | 362 | −4.432 | 105.185 | −69.625 | 1.00 | 47.66 | |
| ATOM | 1555 | O | ILE | | 362 | −4.887 | 106.285 | −69.293 | 1.00 | 48.84 | |
| ATOM | 1556 | N | LEU | | 363 | −3.250 | 104.754 | −69.218 | 1.00 | 46.30 | |
| ATOM | 1557 | CA | LEU | | 363 | −2.485 | 105.578 | −68.326 | 1.00 | 46.39 | |
| ATOM | 1558 | CB | LEU | | 363 | −1.436 | 104.750 | −67.618 | 1.00 | 46.12 | |
| ATOM | 1559 | CG | LEU | | 363 | −2.077 | 103.829 | −66.591 | 1.00 | 48.62 | |
| ATOM | 1560 | CD1 | LEU | | 363 | −1.044 | 102.819 | −66.105 | 1.00 | 47.35 | |
| ATOM | 1561 | CD2 | LEU | | 363 | −2.668 | 104.685 | −65.435 | 1.00 | 45.18 | |
| ATOM | 1562 | C | LEU | | 363 | −1.822 | 106.773 | −68.952 | 1.00 | 47.90 | |
| ATOM | 1563 | O | LEU | | 363 | −1.711 | 107.804 | −68.299 | 1.00 | 50.11 | |
| ATOM | 1564 | N | MET | | 364 | −1.396 | 106.674 | −70.206 | 1.00 | 48.50 | |
| ATOM | 1565 | CA | MET | | 364 | −0.676 | 107.800 | −70.782 | 1.00 | 50.98 | |
| ATOM | 1566 | CB | MET | | 364 | 0.834 | 107.530 | −70.714 | 1.00 | 50.87 | |
| ATOM | 1567 | CG | MET | | 364 | 1.317 | 107.145 | −69.322 | 1.00 | 53.98 | |
| ATOM | 1568 | SD | MET | | 364 | 3.045 | 106.570 | −69.216 | 1.00 | 59.08 | |
| ATOM | 1569 | CE | MET | | 364 | 2.882 | 104.919 | −69.800 | 1.00 | 56.15 | |
| ATOM | 1570 | C | MET | | 364 | −1.036 | 108.234 | −72.179 | 1.00 | 52.24 | |
| ATOM | 1571 | O | MET | | 364 | −0.392 | 109.111 | −72.721 | 1.00 | 53.82 | |
| ATOM | 1572 | N | GLU | | 365 | −2.055 | 107.643 | −72.770 | 1.00 | 53.37 | |
| ATOM | 1573 | CA | GLU | | 365 | −2.422 | 108.045 | −74.115 | 1.00 | 57.40 | |
| ATOM | 1574 | CB | GLU | | 365 | −2.799 | 106.818 | −74.933 | 1.00 | 60.60 | |
| ATOM | 1575 | CG | GLU | | 365 | −3.279 | 107.139 | −76.310 | 1.00 | 64.02 | |
| ATOM | 1576 | CD | GLU | | 365 | −2.184 | 107.689 | −77.168 | 1.00 | 68.42 | |
| ATOM | 1577 | OE1 | GLU | | 365 | −1.590 | 108.721 | −76.783 | 1.00 | 70.32 | |
| ATOM | 1578 | OE2 | GLU | | 365 | −1.918 | 107.084 | −78.232 | 1.00 | 72.08 | |
| ATOM | 1579 | C | GLU | | 365 | −3.590 | 109.021 | −74.096 | 1.00 | 58.80 | |
| ATOM | 1580 | O | GLU | | 365 | −4.532 | 108.851 | −73.331 | 1.00 | 56.60 | |
| ATOM | 1581 | N | GLU | | 366 | −3.543 | 110.043 | −74.942 | 1.00 | 60.64 | |
| ATOM | 1582 | CA | GLU | | 366 | −4.634 | 110.997 | −74.967 | 1.00 | 61.98 | |
| ATOM | 1583 | CB | GLU | | 366 | −4.179 | 112.310 | −75.567 | 1.00 | 65.32 | |
| ATOM | 1584 | CG | GLU | | 366 | −3.699 | 112.176 | −76.996 | 1.00 | 71.72 | |
| ATOM | 1585 | CD | GLU | | 366 | −3.615 | 113.524 | −77.709 | 1.00 | 75.29 | |
| ATOM | 1586 | OE1 | GLU | | 366 | −4.674 | 114.200 | −77.851 | 1.00 | 75.91 | |
| ATOM | 1587 | OE2 | GLU | | 366 | −2.488 | 113.903 | −78.118 | 1.00 | 76.74 | |
| ATOM | 1588 | C | GLU | | 366 | −5.771 | 110.435 | −75.796 | 1.00 | 60.77 | |
| ATOM | 1589 | O | GLU | | 366 | −5.580 | 110.022 | −76.938 | 1.00 | 61.00 | |
| ATOM | 1590 | N | ILE | | 367 | −6.960 | 110.402 | −75.220 | 1.00 | 58.76 | |
| ATOM | 1591 | CA | ILE | | 367 | −8.088 | 109.891 | −75.959 | 1.00 | 58.98 | |
| ATOM | 1592 | CB | ILE | | 367 | −9.241 | 109.558 | −75.025 | 1.00 | 56.06 | |
| ATOM | 1593 | CG2 | ILE | | 367 | −10.427 | 109.109 | −75.822 | 1.00 | 53.24 | |
| ATOM | 1594 | CG1 | ILE | | 367 | −8.795 | 108.470 | −74.037 | 1.00 | 55.06 | |
| ATOM | 1595 | CD1 | ILE | | 367 | −9.794 | 108.162 | −72.932 | 1.00 | 52.27 | |
| ATOM | 1596 | C | ILE | | 367 | −8.508 | 110.950 | −76.971 | 1.00 | 61.65 | |
| ATOM | 1597 | O | ILE | | 367 | −8.640 | 112.130 | −76.635 | 1.00 | 64.27 | |
| ATOM | 1598 | N | ARG | | 368 | −8.705 | 110.516 | −78.212 | 1.00 | 63.12 | |
| ATOM | 1599 | CA | ARG | | 368 | −9.096 | 111.383 | −79.312 | 1.00 | 64.31 | |
| ATOM | 1600 | CB | ARG | | 368 | −8.247 | 111.044 | −80.553 | 1.00 | 68.59 | |
| ATOM | 1601 | CG | ARG | | 368 | −8.693 | 111.737 | −81.842 | 1.00 | 76.42 | |
| ATOM | 1602 | CD | ARG | | 368 | −7.540 | 112.463 | −82.591 | 1.00 | 82.03 | |
| ATOM | 1603 | NE | ARG | | 368 | −6.996 | 113.629 | −81.879 | 1.00 | 85.84 | |
| ATOM | 1604 | CZ | ARG | | 368 | −6.175 | 114.527 | −82.427 | 1.00 | 87.53 | |
| ATOM | 1605 | NH1 | ARG | | 368 | −5.802 | 114.405 | −83.699 | 1.00 | 88.04 | |
| ATOM | 1606 | NH2 | ARG | | 368 | −5.715 | 115.542 | −81.703 | 1.00 | 87.35 | |
| ATOM | 1607 | C | ARG | | 368 | −10.587 | 111.204 | −79.603 | 1.00 | 63.44 | |
| ATOM | 1608 | O | ARG | | 368 | −11.112 | 110.099 | −79.493 | 1.00 | 63.88 | |
| ATOM | 1609 | N | PHE | | 369 | −11.266 | 112.284 | −79.987 | 1.00 | 61.31 | |
| ATOM | 1610 | CA | PHE | | 369 | −12.700 | 112.224 | −80.247 | 1.00 | 59.41 | |
| ATOM | 1611 | CB | PHE | | 369 | −13.449 | 113.249 | −79.380 | 1.00 | 58.28 | |
| ATOM | 1612 | CG | PHE | | 369 | −13.006 | 113.275 | −77.944 | 1.00 | 54.21 | |
| ATOM | 1613 | CD1 | PHE | | 369 | −13.448 | 112.310 | −77.046 | 1.00 | 52.84 | |
| ATOM | 1614 | CD2 | PHE | | 369 | −12.102 | 114.232 | −77.506 | 1.00 | 53.02 | |
| ATOM | 1615 | CE1 | PHE | | 369 | −12.996 | 112.295 | −75.742 | 1.00 | 49.33 | |
| ATOM | 1616 | CE2 | PHE | | 369 | −11.641 | 114.223 | −76.203 | 1.00 | 51.92 | |
| ATOM | 1617 | CZ | PHE | | 369 | −12.091 | 113.248 | −75.316 | 1.00 | 49.73 | |
| ATOM | 1618 | C | PHE | | 369 | −13.056 | 112.503 | −81.684 | 1.00 | 58.66 | |
| ATOM | 1619 | O | PHE | | 369 | −12.484 | 113.378 | −82.314 | 1.00 | 58.34 | |
| ATOM | 1620 | N | PRO | | 370 | −14.018 | 111.765 | −82.221 | 1.00 | 58.55 | |
| ATOM | 1621 | CD | PRO | | 370 | −14.726 | 110.621 | −81.635 | 1.00 | 58.83 | |
| ATOM | 1622 | CA | PRO | | 370 | −14.425 | 111.988 | −83.606 | 1.00 | 61.42 | |
| ATOM | 1623 | CB | PRO | | 370 | −15.632 | 111.076 | −83.752 | 1.00 | 60.56 | |
| ATOM | 1624 | CG | PRO | | 370 | −15.276 | 109.935 | −82.848 | 1.00 | 60.53 | |
| ATOM | 1625 | C | PRO | | 370 | −14.772 | 113.467 | −83.807 | 1.00 | 63.94 | |
| ATOM | 1626 | O | PRO | | 370 | −15.074 | 114.171 | −82.841 | 1.00 | 65.21 | |
| ATOM | 1627 | N | ARG | | 371 | −14.730 | 113.934 | −85.054 | 1.00 | 66.21 | |
| ATOM | 1628 | CA | ARG | | 371 | −15.027 | 115.334 | −85.362 | 1.00 | 66.42 | |
| ATOM | 1629 | CB | ARG | | 371 | −14.433 | 115.724 | −86.720 | 1.00 | 69.22 | |
| ATOM | 1630 | CG | ARG | | 371 | −12.920 | 115.489 | −86.879 | 1.00 | 72.19 | |
| ATOM | 1631 | CD | ARG | | 371 | −12.432 | 115.882 | −88.296 | 1.00 | 74.81 | |
| ATOM | 1632 | NE | ARG | | 371 | −13.090 | 115.121 | −89.369 | 1.00 | 76.73 | |
| ATOM | 1633 | CZ | ARG | | 371 | −12.692 | 113.928 | −89.811 | 1.00 | 78.18 | |
| ATOM | 1634 | NH1 | ARG | | 371 | −11.621 | 113.336 | −89.287 | 1.00 | 78.15 | |
| ATOM | 1635 | NH2 | ARG | | 371 | −13.377 | 113.318 | −90.772 | 1.00 | 78.08 | |
| ATOM | 1636 | C | ARG | | 371 | −16.529 | 115.571 | −85.400 | 1.00 | 65.40 | |
| ATOM | 1637 | O | ARG | | 371 | −17.004 | 116.656 | −85.084 | 1.00 | 65.17 | |
| ATOM | 1638 | N | THR | | 372 | −17.265 | 114.540 | −85.780 | 1.00 | 64.11 | |
| ATOM | 1639 | CA | THR | | 372 | −18.718 | 114.599 | −85.891 | 1.00 | 64.88 | |
| ATOM | 1640 | CB | THR | | 372 | −19.194 | 113.370 | −86.716 | 1.00 | 65.04 | |
| ATOM | 1641 | OG1 | THR | | 372 | −20.506 | 113.612 | −87.236 | 1.00 | 68.26 | |
| ATOM | 1642 | CG2 | THR | | 372 | −19.199 | 112.106 | −85.863 | 1.00 | 63.65 | |
| ATOM | 1643 | C | THR | | 372 | −19.466 | 114.661 | −84.531 | 1.00 | 65.27 | |
| ATOM | 1644 | O | THR | | 372 | −20.667 | 114.390 | −84.442 | 1.00 | 64.03 | |
| ATOM | 1645 | N | LEU | | 373 | −18.754 | 115.036 | −83.473 | 1.00 | 66.41 | |
| ATOM | 1646 | CA | LEU | | 373 | −19.341 | 115.107 | −82.135 | 1.00 | 65.61 | |
| ATOM | 1647 | CB | LEU | | 373 | −18.399 | 114.482 | −81.108 | 1.00 | 64.92 | |
| ATOM | 1648 | CG | LEU | | 373 | −18.850 | 113.336 | −80.194 | 1.00 | 64.67 | |
| ATOM | 1649 | CD1 | LEU | | 373 | −19.581 | 112.265 | −80.975 | 1.00 | 63.41 | |
| ATOM | 1650 | CD2 | LEU | | 373 | −17.618 | 112.756 | −79.519 | 1.00 | 63.52 | |
| ATOM | 1651 | C | LEU | | 373 | −19.593 | 116.530 | −81.724 | 1.00 | 65.19 | |
| ATOM | 1652 | O | LEU | | 373 | −18.703 | 117.370 | −81.812 | 1.00 | 63.80 | |
| ATOM | 1653 | N | SER | | 374 | −20.810 | 116.799 | −81.271 | 1.00 | 65.94 | |
| ATOM | 1654 | CA | SER | | 374 | −21.147 | 118.132 | −80.809 | 1.00 | 65.15 | |
| ATOM | 1655 | CB | SER | | 374 | −22.511 | 118.123 | −80.115 | 1.00 | 64.69 | |
| ATOM | 1656 | OG | SER | | 374 | −22.419 | 117.514 | −78.841 | 1.00 | 63.95 | |
| ATOM | 1657 | C | SER | | 374 | −20.051 | 118.457 | −79.802 | 1.00 | 63.92 | |
| ATOM | 1658 | O | SER | | 374 | −19.584 | 117.575 | −79.093 | 1.00 | 65.77 | |
| ATOM | 1659 | N | PRO | | 375 | −19.624 | 119.720 | −79.731 | 1.00 | 63.66 | |
| ATOM | 1660 | CD | PRO | | 375 | −20.142 | 120.861 | −80.505 | 1.00 | 63.53 | |
| ATOM | 1661 | CA | PRO | | 375 | −18.572 | 120.149 | −78.805 | 1.00 | 62.49 | |
| ATOM | 1662 | CB | PRO | | 375 | −18.295 | 121.579 | −79.249 | 1.00 | 62.86 | |
| ATOM | 1663 | CG | PRO | | 375 | −19.645 | 122.031 | −79.705 | 1.00 | 63.02 | |
| ATOM | 1664 | C | PRO | | 375 | −18.945 | 120.045 | −77.327 | 1.00 | 62.04 | |
| ATOM | 1665 | O | PRO | | 375 | −18.074 | 120.040 | −76.461 | 1.00 | 61.06 | |
| ATOM | 1666 | N | GLU | | 376 | −20.233 | 119.988 | −77.017 | 1.00 | 61.15 | |
| ATOM | 1667 | CA | GLU | | 376 | −20.599 | 119.833 | −75.614 | 1.00 | 61.95 | |
| ATOM | 1668 | CB | GLU | | 376 | −22.081 | 120.133 | −75.386 | 1.00 | 62.71 | |
| ATOM | 1669 | CG | GLU | | 376 | −23.019 | 119.521 | −76.405 | 1.00 | 65.90 | |
| ATOM | 1670 | CD | GLU | | 376 | −23.304 | 120.456 | −77.579 | 1.00 | 67.70 | |
| ATOM | 1671 | OE1 | GLU | | 376 | −22.372 | 121.159 | −78.046 | 1.00 | 68.57 | |
| ATOM | 1672 | OE2 | GLU | | 376 | −24.465 | 120.470 | −78.039 | 1.00 | 67.01 | |
| ATOM | 1673 | C | GLU | | 376 | −20.287 | 118.372 | −75.263 | 1.00 | 61.17 | |
| ATOM | 1674 | O | GLU | | 376 | −19.558 | 118.091 | −74.301 | 1.00 | 60.59 | |
| ATOM | 1675 | N | ALA | | 377 | −20.830 | 117.456 | −76.073 | 1.00 | 59.85 | |
| ATOM | 1676 | CA | ALA | | 377 | −20.603 | 116.017 | −75.905 | 1.00 | 57.71 | |
| ATOM | 1677 | CB | ALA | | 377 | −21.238 | 115.249 | −77.037 | 1.00 | 57.77 | |
| ATOM | 1678 | C | ALA | | 377 | −19.106 | 115.764 | −75.893 | 1.00 | 55.75 | |
| ATOM | 1679 | O | ALA | | 377 | −18.605 | 114.949 | −75.132 | 1.00 | 52.94 | |
| ATOM | 1680 | N | LYS | | 378 | −18.387 | 116.480 | −76.741 | 1.00 | 56.05 | |
| ATOM | 1681 | CA | LYS | | 378 | −16.956 | 116.313 | −76.767 | 1.00 | 57.51 | |
| ATOM | 1682 | CB | LYS | | 378 | −16.337 | 117.087 | −77.935 | 1.00 | 59.91 | |
| ATOM | 1683 | CG | LYS | | 378 | −14.802 | 116.987 | −77.963 | 1.00 | 63.45 | |
| ATOM | 1684 | CD | LYS | | 378 | −14.169 | 117.946 | −78.970 | 1.00 | 65.36 | |
| ATOM | 1685 | CE | LYS | | 378 | −12.635 | 117.887 | −78.920 | 1.00 | 67.38 | |
| ATOM | 1686 | NZ | LYS | | 378 | −11.967 | 118.978 | −79.722 | 1.00 | 67.76 | |
| ATOM | 1687 | C | LYS | | 378 | −16.389 | 116.803 | −75.444 | 1.00 | 55.87 | |
| ATOM | 1688 | O | LYS | | 378 | −15.407 | 116.257 | −74.937 | 1.00 | 58.72 | |
| ATOM | 1689 | N | SER | | 379 | −17.009 | 117.822 | −74.862 | 1.00 | 54.70 | |
| ATOM | 1690 | CA | SER | | 379 | −16.511 | 118.359 | −73.594 | 1.00 | 52.54 | |
| ATOM | 1691 | CB | SER | | 379 | −17.189 | 119.703 | −73.277 | 1.00 | 53.44 | |
| ATOM | 1692 | OG | SER | | 379 | −16.486 | 120.400 | −72.248 | 1.00 | 53.25 | |
| ATOM | 1693 | C | SER | | 379 | −16.755 | 117.378 | −72.444 | 1.00 | 50.71 | |
| ATOM | 1694 | O | SER | | 379 | −15.905 | 117.184 | −71.561 | 1.00 | 47.73 | |
| ATOM | 1695 | N | LEU | | 380 | −17.936 | 116.772 | −72.459 | 1.00 | 48.78 | |
| ATOM | 1696 | CA | LEU | | 380 | −18.301 | 115.814 | −71.435 | 1.00 | 47.66 | |
| ATOM | 1697 | CB | LEU | | 380 | −19.698 | 115.280 | −71.736 | 1.00 | 47.18 | |
| ATOM | 1698 | CG | LEU | | 380 | −20.295 | 114.313 | −70.726 | 1.00 | 46.47 | |
| ATOM | 1699 | CD1 | LEU | | 380 | −20.622 | 115.048 | −69.442 | 1.00 | 45.13 | |
| ATOM | 1700 | CD2 | LEU | | 380 | −21.527 | 113.672 | −71.322 | 1.00 | 44.51 | |
| ATOM | 1701 | C | LEU | | 380 | −17.280 | 114.668 | −71.401 | 1.00 | 47.62 | |
| ATOM | 1702 | O | LEU | | 380 | −16.659 | 114.385 | −70.364 | 1.00 | 45.83 | |
| ATOM | 1703 | N | LEU | | 381 | −17.099 | 114.023 | −72.549 | 1.00 | 47.58 | |
| ATOM | 1704 | CA | LEU | | 381 | −16.169 | 112.908 | −72.654 | 1.00 | 46.38 | |
| ATOM | 1705 | CB | LEU | | 381 | −16.132 | 112.417 | −74.082 | 1.00 | 46.15 | |
| ATOM | 1706 | CG | LEU | | 381 | −17.520 | 111.971 | −74.502 | 1.00 | 47.47 | |
| ATOM | 1707 | CD1 | LEU | | 381 | −17.548 | 111.593 | −75.970 | 1.00 | 46.79 | |
| ATOM | 1708 | CD2 | LEU | | 381 | −17.942 | 110.808 | −73.624 | 1.00 | 47.47 | |
| ATOM | 1709 | C | LEU | | 381 | −14.786 | 113.316 | −72.213 | 1.00 | 46.18 | |
| ATOM | 1710 | O | LEU | | 381 | −14.136 | 112.620 | −71.441 | 1.00 | 47.28 | |
| ATOM | 1711 | N | ALA | | 382 | −14.324 | 114.460 | −72.683 | 1.00 | 47.23 | |
| ATOM | 1712 | CA | ALA | | 382 | −12.991 | 114.881 | −72.289 | 1.00 | 47.82 | |
| ATOM | 1713 | CB | ALA | | 382 | −12.627 | 116.208 | −72.945 | 1.00 | 47.82 | |
| ATOM | 1714 | C | ALA | | 382 | −12.938 | 114.997 | −70.781 | 1.00 | 46.91 | |
| ATOM | 1715 | O | ALA | | 382 | −11.916 | 114.713 | −70.176 | 1.00 | 48.30 | |
| ATOM | 1716 | N | GLY | | 383 | −14.055 | 115.391 | −70.171 | 1.00 | 47.90 | |
| ATOM | 1717 | CA | GLY | | 383 | −14.082 | 115.542 | −68.720 | 1.00 | 45.67 | |
| ATOM | 1718 | C | GLY | | 383 | −14.214 | 114.249 | −67.922 | 1.00 | 44.89 | |
| ATOM | 1719 | O | GLY | | 383 | −13.576 | 114.063 | −66.873 | 1.00 | 42.07 | |
| ATOM | 1720 | N | LEU | | 384 | −15.066 | 113.357 | −68.405 | 1.00 | 43.92 | |
| ATOM | 1721 | CA | LEU | | 384 | −15.252 | 112.092 | −67.732 | 1.00 | 45.87 | |
| ATOM | 1722 | CB | LEU | | 384 | −16.458 | 111.368 | −68.326 | 1.00 | 42.85 | |
| ATOM | 1723 | CG | LEU | | 384 | −17.816 | 112.028 | −68.087 | 1.00 | 39.24 | |
| ATOM | 1724 | CD1 | LEU | | 384 | −18.874 | 111.269 | −68.856 | 1.00 | 38.96 | |
| ATOM | 1725 | CD2 | LEU | | 384 | −18.138 | 112.047 | −66.590 | 1.00 | 40.88 | |
| ATOM | 1726 | C | LEU | | 384 | −13.997 | 111.231 | −67.894 | 1.00 | 48.25 | |
| ATOM | 1727 | O | LEU | | 384 | −13.687 | 110.401 | −67.038 | 1.00 | 50.20 | |
| ATOM | 1728 | N | LEU | | 385 | −13.262 | 111.434 | −68.980 | 1.00 | 49.92 | |
| ATOM | 1729 | CA | LEU | | 385 | −12.077 | 110.630 | −69.207 | 1.00 | 50.81 | |
| ATOM | 1730 | CB | LEU | | 385 | −11.973 | 110.246 | −70.670 | 1.00 | 50.57 | |
| ATOM | 1731 | CG | LEU | | 385 | −13.207 | 109.482 | −71.145 | 1.00 | 51.27 | |
| ATOM | 1732 | CD1 | LEU | | 385 | −13.112 | 109.264 | −72.648 | 1.00 | 54.08 | |
| ATOM | 1733 | CD2 | LEU | | 385 | −13.322 | 108.158 | −70.413 | 1.00 | 51.68 | |
| ATOM | 1734 | C | LEU | | 385 | −10.778 | 111.246 | −68.749 | 1.00 | 51.61 | |
| ATOM | 1735 | O | LEU | | 385 | −9.735 | 110.850 | −69.221 | 1.00 | 53.44 | |
| ATOM | 1736 | N | LYS | | 386 | −10.832 | 112.209 | −67.836 | 1.00 | 52.32 | |
| ATOM | 1737 | CA | LYS | | 386 | −9.614 | 112.806 | −67.296 | 1.00 | 54.25 | |
| ATOM | 1738 | CB | LYS | | 386 | −9.964 | 113.930 | −66.312 | 1.00 | 55.53 | |
| ATOM | 1739 | CG | LYS | | 386 | −9.973 | 115.337 | −66.890 | 1.00 | 59.35 | |
| ATOM | 1740 | CD | LYS | | 386 | −8.586 | 115.737 | −67.379 | 1.00 | 61.57 | |
| ATOM | 1741 | CE | LYS | | 386 | −8.476 | 117.225 | −67.729 | 1.00 | 62.40 | |
| ATOM | 1742 | NZ | LYS | | 386 | −8.110 | 118.106 | −66.554 | 1.00 | 64.25 | |
| ATOM | 1743 | C | LYS | | 386 | −8.837 | 111.689 | −66.563 | 1.00 | 55.28 | |
| ATOM | 1744 | O | LYS | | 386 | −9.412 | 110.912 | −65.797 | 1.00 | 55.21 | |
| ATOM | 1745 | N | LYS | | 387 | −7.532 | 111.614 | −66.783 | 1.00 | 56.62 | |
| ATOM | 1746 | CA | LYS | | 387 | −6.734 | 110.567 | −66.161 | 1.00 | 57.65 | |
| ATOM | 1747 | CB | LYS | | 387 | −5.431 | 110.371 | −66.941 | 1.00 | 57.20 | |
| ATOM | 1748 | CG | LYS | | 387 | −5.629 | 109.607 | −68.233 | 1.00 | 54.57 | |
| ATOM | 1749 | CD | LYS | | 387 | −4.520 | 109.849 | −69.228 | 1.00 | 57.32 | |
| ATOM | 1750 | CE | LYS | | 387 | −4.495 | 108.784 | −70.325 | 1.00 | 56.45 | |
| ATOM | 1751 | NZ | LYS | | 387 | −5.787 | 108.581 | −71.027 | 1.00 | 54.02 | |
| ATOM | 1752 | C | LYS | | 387 | −6.457 | 110.808 | −64.700 | 1.00 | 58.92 | |
| ATOM | 1753 | O | LYS | | 387 | −6.108 | 109.891 | −63.970 | 1.00 | 58.52 | |
| ATOM | 1754 | N | ASP | | 388 | −6.622 | 112.050 | −64.270 | 1.00 | 61.81 | |
| ATOM | 1755 | CA | ASP | | 388 | −6.399 | 112.402 | −62.872 | 1.00 | 62.16 | |
| ATOM | 1756 | CB | ASP | | 388 | −5.855 | 113.821 | −62.746 | 1.00 | 62.17 | |
| ATOM | 1757 | CG | ASP | | 388 | −5.111 | 114.047 | −61.446 | 1.00 | 61.91 | |
| ATOM | 1758 | OD1 | ASP | | 388 | −5.446 | 113.405 | −60.428 | 1.00 | 60.36 | |
| ATOM | 1759 | OD2 | ASP | | 388 | −4.187 | 114.888 | −61.447 | 1.00 | 65.14 | |
| ATOM | 1760 | C | ASP | | 388 | −7.754 | 112.357 | −62.193 | 1.00 | 63.15 | |
| ATOM | 1761 | O | ASP | | 388 | −8.635 | 113.159 | −62.520 | 1.00 | 62.45 | |
| ATOM | 1762 | N | PRO | | 389 | −7.953 | 111.417 | −61.250 | 1.00 | 63.99 | |
| ATOM | 1763 | CD | PRO | | 389 | −7.053 | 110.472 | −60.564 | 1.00 | 63.69 | |
| ATOM | 1764 | CA | PRO | | 389 | −9.278 | 111.427 | −60.630 | 1.00 | 64.65 | |
| ATOM | 1765 | CB | PRO | | 389 | −9.157 | 110.368 | −59.527 | 1.00 | 63.51 | |
| ATOM | 1766 | CG | PRO | | 389 | −7.699 | 110.350 | −59.214 | 1.00 | 63.39 | |
| ATOM | 1767 | C | PRO | | 389 | −9.550 | 112.833 | −60.111 | 1.00 | 64.89 | |
| ATOM | 1768 | O | PRO | | 389 | −10.602 | 113.406 | −60.379 | 1.00 | 65.71 | |
| ATOM | 1769 | N | LYS | | 390 | −8.574 | 113.413 | −59.425 | 1.00 | 64.17 | |
| ATOM | 1770 | CA | LYS | | 390 | −8.755 | 114.750 | −58.896 | 1.00 | 65.12 | |
| ATOM | 1771 | CB | LYS | | 390 | −7.502 | 115.210 | −58.162 | 1.00 | 66.88 | |
| ATOM | 1772 | CG | LYS | | 390 | −7.480 | 114.745 | −56.723 | 1.00 | 69.54 | |
| ATOM | 1773 | CD | LYS | | 390 | −6.348 | 115.373 | −55.924 | 1.00 | 74.47 | |
| ATOM | 1774 | CE | LYS | | 390 | −6.468 | 115.015 | −54.438 | 1.00 | 77.11 | |
| ATOM | 1775 | NZ | LYS | | 390 | −6.509 | 113.535 | −54.182 | 1.00 | 78.67 | |
| ATOM | 1776 | C | LYS | | 390 | −9.181 | 115.812 | −59.895 | 1.00 | 64.23 | |
| ATOM | 1777 | O | LYS | | 390 | −9.512 | 116.927 | −59.501 | 1.00 | 64.56 | |
| ATOM | 1778 | N | GLN | | 391 | −9.187 | 115.490 | −61.181 | 1.00 | 62.47 | |
| ATOM | 1779 | CA | GLN | | 391 | −9.611 | 116.485 | −62.162 | 1.00 | 60.75 | |
| ATOM | 1780 | CB | GLN | | 391 | −8.477 | 116.866 | −63.084 | 1.00 | 62.53 | |
| ATOM | 1781 | CG | GLN | | 391 | −7.246 | 117.347 | −62.412 | 1.00 | 67.86 | |
| ATOM | 1782 | CD | GLN | | 391 | −6.152 | 117.548 | −63.434 | 1.00 | 73.05 | |
| ATOM | 1783 | OE1 | GLN | | 391 | −6.320 | 118.332 | −64.388 | 1.00 | 74.71 | |
| ATOM | 1784 | NE2 | GLN | | 391 | −5.025 | 116.833 | −63.263 | 1.00 | 73.43 | |
| ATOM | 1785 | C | CLN | | 391 | −10.744 | 115.994 | −63.032 | 1.00 | 58.42 | |
| ATOM | 1786 | O | GLN | | 391 | −11.208 | 116.711 | −63.919 | 1.00 | 58.65 | |
| ATOM | 1787 | N | ARG | | 392 | −11.193 | 114.774 | −62.797 | 1.00 | 54.84 | |
| ATOM | 1788 | CA | ARG | | 392 | −12.259 | 114.246 | −63.618 | 1.00 | 52.67 | |
| ATOM | 1789 | CB | ARG | | 392 | −12.384 | 112.745 | −63.396 | 1.00 | 49.94 | |
| ATOM | 1790 | CG | ARG | | 392 | −13.290 | 112.012 | −64.376 | 1.00 | 48.29 | |
| ATOM | 1791 | CD | ARG | | 392 | −13.054 | 110.500 | −64.206 | 1.00 | 48.02 | |
| ATOM | 1792 | NE | ARG | | 392 | −11.617 | 110.210 | −64.177 | 1.00 | 46.31 | |
| ATOM | 1793 | CZ | ARG | | 392 | −11.052 | 109.274 | −63.426 | 1.00 | 44.20 | |
| ATOM | 1794 | NH1 | ARG | | 392 | −11.801 | 108.520 | −62.636 | 1.00 | 44.34 | |
| ATOM | 1795 | NH2 | ARG | | 392 | −9.737 | 109.121 | −63.440 | 1.00 | 42.60 | |
| ATOM | 1796 | C | ARG | | 392 | −13.588 | 114.932 | −63.344 | 1.00 | 50.78 | |
| ATOM | 1797 | O | ARG | | 392 | −13.861 | 115.393 | −62.251 | 1.00 | 52.37 | |
| ATOM | 1798 | N | LEU | | 393 | −14.399 | 115.016 | −64.375 | 1.00 | 49.90 | |
| ATOM | 1799 | CA | LEU | | 393 | −15.709 | 115.586 | −64.256 | 1.00 | 50.17 | |
| ATOM | 1800 | CB | LEU | | 393 | −16.406 | 115.541 | −65.610 | 1.00 | 48.71 | |
| ATOM | 1801 | CG | LEU | | 393 | −17.595 | 116.450 | −65.879 | 1.00 | 47.91 | |
| ATOM | 1802 | CD1 | LEU | | 393 | −18.567 | 115.731 | −66.796 | 1.00 | 47.02 | |
| ATOM | 1803 | CD2 | LEU | | 393 | −18.259 | 116.816 | −64.576 | 1.00 | 49.63 | |
| ATOM | 1804 | C | LEU | | 393 | −16.432 | 114.653 | −63.288 | 1.00 | 52.41 | |
| ATOM | 1805 | O | LEU | | 393 | −16.839 | 113.549 | −63.671 | 1.00 | 53.23 | |
| ATOM | 1806 | N | GLY | | 394 | −16.581 | 115.081 | −62.037 | 1.00 | 53.32 | |
| ATOM | 1807 | CA | GLY | | 394 | −17.277 | 114.261 | −61.070 | 1.00 | 53.52 | |
| ATOM | 1808 | C | GLY | | 394 | −16.340 | 113.821 | −59.981 | 1.00 | 54.11 | |
| ATOM | 1809 | O | GLY | | 394 | −16.772 | 113.291 | −58.956 | 1.00 | 53.36 | |
| ATOM | 1810 | N | GLY | | 395 | −15.053 | 114.047 | −60.214 | 1.00 | 54.86 | |
| ATOM | 1811 | CA | GLY | | 395 | −14.040 | 113.678 | −59.248 | 1.00 | 56.24 | |
| ATOM | 1812 | C | GLY | | 395 | −13.876 | 114.765 | −58.201 | 1.00 | 58.65 | |
| ATOM | 1813 | O | GLY | | 395 | −13.037 | 114.656 | −57.297 | 1.00 | 59.43 | |
| ATOM | 1814 | N | GLY | | 396 | −14.664 | 115.828 | −58.329 | 1.00 | 59.28 | |
| ATOM | 1815 | CA | GLY | | 396 | −14.597 | 116.912 | −57.362 | 1.00 | 59.27 | |
| ATOM | 1816 | C | GLY | | 396 | −15.568 | 116.660 | −56.218 | 1.00 | 59.64 | |
| ATOM | 1817 | O | GLY | | 396 | −16.538 | 115.920 | −56.398 | 1.00 | 58.40 | |
| ATOM | 1818 | N | PRO | | 397 | −15.347 | 117.270 | −55.031 | 1.00 | 59.85 | |
| ATOM | 1819 | CD | PRO | | 397 | −14.322 | 118.293 | −54.770 | 1.00 | 59.44 | |
| ATOM | 1820 | CA | PRO | | 397 | −16.213 | 117.098 | −53.853 | 1.00 | 59.00 | |
| ATOM | 1821 | CB | PRO | | 397 | −15.695 | 118.155 | −52.878 | 1.00 | 59.15 | |
| ATOM | 1822 | CG | PRO | | 397 | −15.006 | 119.161 | −53.762 | 1.00 | 59.13 | |
| ATOM | 1823 | C | PRO | | 397 | −17.711 | 117.212 | −54.095 | 1.00 | 59.00 | |
| ATOM | 1824 | O | PRO | | 397 | −18.514 | 116.724 | −53.298 | 1.00 | 59.48 | |
| ATOM | 1825 | N | SER | | 398 | −18.084 | 117.841 | −55.201 | 1.00 | 58.44 | |
| ATOM | 1826 | CA | SER | | 398 | −19.479 | 118.013 | −55.542 | 1.00 | 58.58 | |
| ATOM | 1827 | CB | SER | | 398 | −19.636 | 119.226 | −56.451 | 1.00 | 62.15 | |
| ATOM | 1828 | OG | SER | | 398 | −20.991 | 119.394 | −56.846 | 1.00 | 66.14 | |
| ATOM | 1829 | C | SER | | 398 | −20.024 | 116.781 | −56.247 | 1.00 | 57.44 | |
| ATOM | 1830 | O | SER | | 398 | −21.188 | 116.754 | −56.642 | 1.00 | 56.72 | |
| ATOM | 1831 | N | ASP | | 399 | −19.167 | 115.777 | −56.419 | 1.00 | 56.57 | |
| ATOM | 1832 | CA | ASP | | 399 | −19.527 | 114.515 | −57.076 | 1.00 | 54.02 | |
| ATOM | 1833 | CB | ASP | | 399 | −20.107 | 113.528 | −56.041 | 1.00 | 54.67 | |
| ATOM | 1834 | CG | ASP | | 399 | −20.177 | 112.096 | −56.558 | 1.00 | 53.89 | |
| ATOM | 1835 | OD1 | ASP | | 399 | −21.288 | 111.628 | −56.860 | 1.00 | 55.72 | |
| ATOM | 1836 | OD2 | ASP | | 399 | −19.123 | 111.435 | −56.663 | 1.00 | 53.25 | |
| ATOM | 1837 | C | ASP | | 399 | −20.492 | 114.681 | −58.254 | 1.00 | 52.23 | |
| ATOM | 1838 | O | ASP | | 399 | −20.250 | 115.490 | −59.146 | 1.00 | 51.65 | |
| ATOM | 1839 | N | ALA | | 400 | −21.585 | 113.926 | −58.256 | 1.00 | 50.05 | |
| ATOM | 1840 | CA | ALA | | 400 | −22.541 | 114.000 | −59.350 | 1.00 | 50.76 | |
| ATOM | 1841 | CB | ALA | | 400 | −23.817 | 113.245 | −58.981 | 1.00 | 48.57 | |
| ATOM | 1842 | C | ALA | | 400 | −22.888 | 115.419 | −59.834 | 1.00 | 51.21 | |
| ATOM | 1843 | O | ALA | | 400 | −23.028 | 115.653 | −61.028 | 1.00 | 50.70 | |
| ATOM | 1844 | N | LYS | | 401 | −23.026 | 116.372 | −58.927 | 1.00 | 52.29 | |
| ATOM | 1845 | CA | LYS | | 401 | −23.373 | 117.722 | −59.355 | 1.00 | 54.73 | |
| ATOM | 1846 | CB | LYS | | 401 | −23.317 | 118.664 | −58.159 | 1.00 | 56.85 | |
| ATOM | 1847 | CG | LYS | | 401 | −24.401 | 118.358 | −57.136 | 1.00 | 59.67 | |
| ATOM | 1848 | CD | LYS | | 401 | −24.009 | 118.894 | −55.754 | 1.00 | 63.33 | |
| ATOM | 1849 | CE | LYS | | 401 | −25.081 | 118.675 | −54.699 | 1.00 | 61.16 | |
| ATOM | 1850 | NZ | LYS | | 401 | −24.682 | 119.357 | −53.437 | 1.00 | 60.07 | |
| ATOM | 1851 | C | LYS | | 401 | −22.538 | 118.258 | −60.529 | 1.00 | 53.87 | |
| ATOM | 1852 | O | LYS | | 401 | −23.100 | 118.718 | −61.510 | 1.00 | 54.65 | |
| ATOM | 1853 | N | GLU | | 402 | −21.216 | 118.192 | −60.458 | 1.00 | 54.04 | |
| ATOM | 1854 | CA | GLU | | 402 | −20.424 | 118.663 | −61.587 | 1.00 | 56.21 | |
| ATOM | 1855 | CB | GLU | | 402 | −18.967 | 118.276 | −61.408 | 1.00 | 57.34 | |
| ATOM | 1856 | CG | GLU | | 402 | −18.248 | 118.977 | −60.285 | 1.00 | 61.35 | |
| ATOM | 1857 | CD | GLU | | 402 | −16.812 | 118.470 | −60.133 | 1.00 | 67.08 | |
| ATOM | 1858 | OE1 | GLU | | 402 | −16.136 | 118.249 | −61.168 | 1.00 | 71.46 | |
| ATOM | 1859 | OE2 | GLU | | 402 | −16.343 | 118.295 | −58.988 | 1.00 | 69.45 | |
| ATOM | 1860 | C | GLU | | 402 | −20.930 | 118.078 | −62.918 | 1.00 | 56.01 | |
| ATOM | 1861 | O | GLU | | 402 | −20.962 | 118.757 | −63.939 | 1.00 | 58.61 | |
| ATOM | 1862 | N | VAL | | 403 | −21.339 | 116.818 | −62.896 | 1.00 | 55.01 | |
| ATOM | 1863 | CA | VAL | | 403 | −21.818 | 116.126 | −64.087 | 1.00 | 54.30 | |
| ATOM | 1864 | CB | VAL | | 403 | −21.745 | 114.577 | −63.869 | 1.00 | 52.34 | |
| ATOM | 1865 | CG1 | VAL | | 403 | −22.260 | 113.806 | −65.096 | 1.00 | 49.97 | |
| ATOM | 1866 | CG2 | VAL | | 403 | −20.338 | 114.191 | −63.547 | 1.00 | 48.55 | |
| ATOM | 1867 | C | VAL | | 403 | −23.249 | 116.506 | −64.477 | 1.00 | 55.72 | |
| ATOM | 1868 | O | VAL | | 403 | −23.579 | 116.631 | −65.665 | 1.00 | 55.85 | |
| ATOM | 1869 | N | MET | | 404 | −24.109 | 116.665 | −63.479 | 1.00 | 56.07 | |
| ATOM | 1870 | CA | MET | | 404 | −25.500 | 117.018 | −63.752 | 1.00 | 56.54 | |
| ATOM | 1871 | CB | MET | | 404 | −26.326 | 116.934 | −62.470 | 1.00 | 55.56 | |
| ATOM | 1872 | CG | MET | | 404 | −26.808 | 115.543 | −62.127 | 1.00 | 56.38 | |
| ATOM | 1873 | SD | MET | | 404 | −27.379 | 115.534 | −60.461 | 1.00 | 53.80 | |
| ATOM | 1874 | CE | MET | | 404 | −25.837 | 115.738 | −59.667 | 1.00 | 54.96 | |
| ATOM | 1875 | C | MET | | 404 | −25.635 | 118.418 | −64.339 | 1.00 | 55.41 | |
| ATOM | 1876 | O | MET | | 404 | −26.583 | 118.709 | −65.057 | 1.00 | 53.73 | |
| ATOM | 1877 | N | GLU | | 405 | −24.673 | 119.273 | −64.022 | 1.00 | 55.58 | |
| ATOM | 1878 | CA | GLU | | 405 | −24.688 | 120.640 | −64.483 | 1.00 | 58.04 | |
| ATOM | 1879 | CB | GLU | | 405 | −24.334 | 121.561 | −63.328 | 1.00 | 59.78 | |
| ATOM | 1880 | CG | GLU | | 405 | −25.120 | 121.309 | −62.074 | 1.00 | 63.57 | |
| ATOM | 1881 | CD | GLU | | 405 | −24.559 | 122.097 | −60.910 | 1.00 | 66.97 | |
| ATOM | 1882 | OE1 | GLU | | 405 | −23.353 | 122.442 | −60.977 | 1.00 | 66.24 | |
| ATOM | 1883 | OE2 | GLU | | 405 | −25.309 | 122.359 | −59.937 | 1.00 | 69.08 | |
| ATOM | 1884 | C | GLU | | 405 | −23.707 | 120.896 | −65.620 | 1.00 | 59.79 | |
| ATOM | 1885 | O | GLU | | 405 | −23.284 | 122.046 | −65.837 | 1.00 | 60.33 | |
| ATOM | 1886 | N | HIS | | 406 | −23.319 | 119.845 | −66.341 | 1.00 | 58.09 | |
| ATOM | 1887 | CA | HIS | | 406 | −22.377 | 120.057 | −67.420 | 1.00 | 52.85 | |
| ATOM | 1888 | CB | HIS | | 406 | −21.558 | 118.794 | −67.742 | 1.00 | 48.84 | |
| ATOM | 1889 | CG | HIS | | 406 | −20.579 | 118.978 | −68.864 | 1.00 | 40.92 | |
| ATOM | 1890 | CD2 | HIS | | 406 | −19.263 | 119.296 | −68.855 | 1.00 | 38.08 | |
| ATOM | 1891 | ND1 | HIS | | 406 | −20.938 | 118.885 | −70.196 | 1.00 | 40.72 | |
| ATOM | 1892 | CE1 | HIS | | 406 | −19.889 | 119.136 | −70.953 | 1.00 | 34.51 | |
| ATOM | 1893 | NE2 | HIS | | 406 | −18.859 | 119.391 | −70.165 | 1.00 | 36.37 | |
| ATOM | 1894 | C | HIS | | 406 | −23.146 | 120.483 | −68.628 | 1.00 | 54.12 | |
| ATOM | 1895 | O | HIS | | 406 | −24.302 | 120.093 | −68.838 | 1.00 | 52.18 | |
| ATOM | 1896 | N | ARG | | 407 | −22.479 | 121.321 | −69.404 | 1.00 | 55.55 | |
| ATOM | 1897 | CA | ARG | | 407 | −23.001 | 121.846 | −70.640 | 1.00 | 58.47 | |
| ATOM | 1898 | CB | ARG | | 407 | −21.816 | 122.218 | −71.517 | 1.00 | 62.44 | |
| ATOM | 1899 | CG | ARG | | 407 | −22.120 | 122.712 | −72.902 | 1.00 | 66.15 | |
| ATOM | 1900 | CD | ARG | | 407 | −20.781 | 122.911 | −73.581 | 1.00 | 69.94 | |
| ATOM | 1901 | NE | ARG | | 407 | −20.881 | 123.436 | −74.934 | 1.00 | 75.38 | |
| ATOM | 1902 | CZ | ARG | | 407 | −19.828 | 123.780 | −75.674 | 1.00 | 77.07 | |
| ATOM | 1903 | NH1 | ARG | | 407 | −18.599 | 123.653 | −75.179 | 1.00 | 77.19 | |
| ATOM | 1904 | NH2 | ARG | | 407 | −20.006 | 124.243 | −76.907 | 1.00 | 78.05 | |
| ATOM | 1905 | C | ARG | | 407 | −23.835 | 120.774 | −71.307 | 1.00 | 58.60 | |
| ATOM | 1906 | O | ARG | | 407 | −25.030 | 120.967 | −71.546 | 1.00 | 58.90 | |
| ATOM | 1907 | N | PHE | | 408 | −23.209 | 119.628 | −71.579 | 1.00 | 57.25 | |
| ATOM | 1908 | CA | PHE | | 408 | −23.921 | 118.539 | −72.243 | 1.00 | 56.47 | |
| ATOM | 1909 | CB | PHE | | 408 | −23.056 | 117.267 | −72.290 | 1.00 | 56.76 | |
| ATOM | 1910 | CG | PHE | | 408 | −23.709 | 116.129 | −73.027 | 1.00 | 54.26 | |
| ATOM | 1911 | CD1 | PHE | | 408 | −23.666 | 116.071 | −74.410 | 1.00 | 53.88 | |
| ATOM | 1912 | CD2 | PHE | | 408 | −24.434 | 115.164 | −72.335 | 1.00 | 53.15 | |
| ATOM | 1913 | CE1 | PHE | | 408 | −24.335 | 115.077 | −75.097 | 1.00 | 55.74 | |
| ATOM | 1914 | CE2 | PHE | | 408 | −25.114 | 114.165 | −73.008 | 1.00 | 54.60 | |
| ATOM | 1915 | CZ | PHE | | 408 | −25.067 | 114.117 | −74.398 | 1.00 | 57.66 | |
| ATOM | 1916 | C | PHE | | 408 | −25.255 | 118.221 | −71.566 | 1.00 | 55.42 | |
| ATOM | 1917 | O | PHE | | 408 | −26.179 | 117.726 | −72.205 | 1.00 | 53.41 | |
| ATOM | 1918 | N | PHE | | 409 | −25.348 | 118.516 | −70.271 | 1.00 | 55.79 | |
| ATOM | 1919 | CA | PHE | | 409 | −26.558 | 118.253 | −69.509 | 1.00 | 56.18 | |
| ATOM | 1920 | CB | PHE | | 409 | −26.225 | 117.407 | −68.245 | 1.00 | 53.73 | |
| ATOM | 1921 | CG | PHE | | 409 | −25.870 | 115.940 | −68.528 | 1.00 | 47.14 | |
| ATOM | 1922 | CD1 | PHE | | 409 | −26.801 | 115.075 | −69.107 | 1.00 | 44.58 | |
| ATOM | 1923 | CD2 | PHE | | 409 | −24.616 | 115.442 | −68.213 | 1.00 | 44.43 | |
| ATOM | 1924 | CE1 | PHE | | 409 | −26.482 | 113.748 | −69.367 | 1.00 | 44.98 | |
| ATOM | 1925 | CE2 | PHE | | 409 | −24.277 | 114.093 | −68.471 | 1.00 | 44.11 | |
| ATOM | 1926 | CZ | PHE | | 409 | −25.210 | 113.252 | −69.050 | 1.00 | 42.55 | |
| ATOM | 1927 | C | PHE | | 409 | −27.292 | 119.552 | −69.101 | 1.00 | 59.74 | |
| ATOM | 1928 | O | PHE | | 409 | −27.103 | 120.069 | −67.990 | 1.00 | 59.58 | |
| ATOM | 1929 | N | LEU | | 410 | −28.134 | 120.076 | −69.998 | 1.00 | 61.57 | |
| ATOM | 1930 | CA | LEU | | 410 | −28.922 | 121.279 | −69.712 | 1.00 | 63.20 | |
| ATOM | 1931 | CB | LEU | | 410 | −28.606 | 122.400 | −70.703 | 1.00 | 64.81 | |
| ATOM | 1932 | CG | LEU | | 410 | −29.718 | 123.442 | −70.916 | 1.00 | 64.28 | |
| ATOM | 1933 | CD1 | LEU | | 410 | −29.124 | 124.805 | −71.188 | 1.00 | 63.37 | |
| ATOM | 1934 | CD2 | LEU | | 410 | −30.609 | 123.003 | −72.074 | 1.00 | 62.40 | |
| ATOM | 1935 | C | LEU | | 410 | −30.399 | 120.953 | −69.791 | 1.00 | 63.34 | |
| ATOM | 1936 | O | LEU | | 410 | −31.151 | 121.165 | −68.855 | 1.00 | 64.71 | |
| ATOM | 1937 | N | SER | | 411 | −30.797 | 120.434 | −70.931 | 1.00 | 63.38 | |
| ATOM | 1938 | CA | SER | | 411 | −32.168 | 120.061 | −71.189 | 1.00 | 63.86 | |
| ATOM | 1939 | CB | SER | | 411 | −32.222 | 119.362 | −72.540 | 1.00 | 65.46 | |
| ATOM | 1940 | OG | SER | | 411 | −31.246 | 118.319 | −72.586 | 1.00 | 65.31 | |
| ATOM | 1941 | C | SER | | 411 | −32.747 | 119.124 | −70.141 | 1.00 | 63.97 | |
| ATOM | 1942 | O | SER | | 411 | −33.963 | 118.919 | −70.090 | 1.00 | 65.56 | |
| ATOM | 1943 | N | ILE | | 412 | −31.882 | 118.550 | −69.308 | 1.00 | 63.16 | |
| ATOM | 1944 | CA | ILE | | 412 | −32.324 | 117.576 | −68.315 | 1.00 | 59.72 | |
| ATOM | 1945 | CB | ILE | | 412 | −31.176 | 116.621 | −67.886 | 1.00 | 59.74 | |
| ATOM | 1946 | CG2 | ILE | | 412 | −31.732 | 115.541 | −66.969 | 1.00 | 60.46 | |
| ATOM | 1947 | CG1 | ILE | | 412 | −30.554 | 115.943 | −69.111 | 1.00 | 58.99 | |
| ATOM | 1948 | CD1 | ILE | | 412 | −31.553 | 115.171 | −69.950 | 1.00 | 60.50 | |
| ATOM | 1949 | C | ILE | | 412 | −32.990 | 118.037 | −67.041 | 1.00 | 57.34 | |
| ATOM | 1950 | O | ILE | | 412 | −32.588 | 118.988 | −66.384 | 1.00 | 55.60 | |
| ATOM | 1951 | N | ASN | | 413 | −34.018 | 117.293 | −66.687 | 1.00 | 55.66 | |
| ATOM | 1952 | CA | ASN | | 413 | −34.742 | 117.547 | −65.481 | 1.00 | 54.01 | |
| ATOM | 1953 | CB | ASN | | 413 | −36.209 | 117.699 | −65.813 | 1.00 | 57.97 | |
| ATOM | 1954 | CG | ASN | | 413 | −36.880 | 118.676 | −64.904 | 1.00 | 62.55 | |
| ATOM | 1955 | OD1 | ASN | | 413 | −37.468 | 118.295 | −63.877 | 1.00 | 62.35 | |
| ATOM | 1956 | ND2 | ASN | | 413 | −36.773 | 119.969 | −65.252 | 1.00 | 64.77 | |
| ATOM | 1957 | C | ASN | | 413 | −34.495 | 116.339 | −64.581 | 1.00 | 50.15 | |
| ATOM | 1958 | O | ASN | | 413 | −35.194 | 115.327 | −64.664 | 1.00 | 46.05 | |
| ATOM | 1959 | N | TRP | | 414 | −33.495 | 116.471 | −63.717 | 1.00 | 49.18 | |
| ATOM | 1960 | CA | TRP | | 414 | −33.082 | 115.393 | −62.827 | 1.00 | 50.10 | |
| ATOM | 1961 | CB | TRP | | 414 | −31.928 | 115.864 | −61.957 | 1.00 | 49.14 | |
| ATOM | 1962 | CG | TRP | | 414 | −30.794 | 116.301 | −62.825 | 1.00 | 53.71 | |
| ATOM | 1963 | CD2 | TRP | | 414 | −29.905 | 115.450 | −63.590 | 1.00 | 56.49 | |
| ATOM | 1964 | CE2 | TRP | | 414 | −29.069 | 116.296 | −64.359 | 1.00 | 55.49 | |
| ATOM | 1965 | CE3 | TRP | | 414 | −29.738 | 114.058 | −63.699 | 1.00 | 56.94 | |
| ATOM | 1966 | CD1 | TRP | | 414 | −30.462 | 117.578 | −63.151 | 1.00 | 54.04 | |
| ATOM | 1967 | NE1 | TRP | | 414 | −29.428 | 117.587 | −64.077 | 1.00 | 56.41 | |
| ATOM | 1968 | CZ2 | TRP | | 414 | −28.082 | 115.799 | −65.217 | 1.00 | 56.63 | |
| ATOM | 1969 | CZ3 | TRP | | 414 | −28.746 | 113.561 | −64.563 | 1.00 | 57.11 | |
| ATOM | 1970 | CH2 | TRP | | 414 | −27.936 | 114.434 | −65.307 | 1.00 | 56.43 | |
| ATOM | 1971 | C | TRP | | 414 | −34.167 | 114.753 | −62.008 | 1.00 | 51.80 | |
| ATOM | 1972 | O | TRP | | 414 | −34.085 | 113.566 | −61.690 | 1.00 | 54.04 | |
| ATOM | 1973 | N | GLN | | 415 | −35.204 | 115.516 | −61.682 | 1.00 | 52.38 | |
| ATOM | 1974 | CA | GLN | | 415 | −36.305 | 114.963 | −60.921 | 1.00 | 51.12 | |
| ATOM | 1975 | CB | GLN | | 415 | −37.241 | 116.064 | −60.455 | 1.00 | 50.32 | |
| ATOM | 1976 | CG | GLN | | 415 | −37.102 | 116.392 | −59.004 | 1.00 | 48.51 | |
| ATOM | 1977 | CD | CLN | | 415 | −38.256 | 117.229 | −58.503 | 1.00 | 44.92 | |
| ATOM | 1978 | OE1 | GLN | | 415 | −39.405 | 116.818 | −58.569 | 1.00 | 45.16 | |
| ATOM | 1979 | NE2 | GLN | | 415 | −37.949 | 118.406 | −58.004 | 1.00 | 41.65 | |
| ATOM | 1980 | C | GLN | | 415 | −37.037 | 114.061 | −61.875 | 1.00 | 53.28 | |
| ATOM | 1981 | O | GLN | | 415 | −37.503 | 112.973 | −61.520 | 1.00 | 53.75 | |
| ATOM | 1982 | N | ASP | | 416 | −37.134 | 114.535 | −63.106 | 1.00 | 54.15 | |
| ATOM | 1983 | CA | ASP | | 416 | −37.815 | 113.793 | −64.146 | 1.00 | 55.69 | |
| ATOM | 1984 | CB | ASP | | 416 | −37.837 | 114.613 | −65.442 | 1.00 | 60.37 | |
| ATOM | 1985 | CG | ASP | | 416 | −38.998 | 115.599 | −65.490 | 1.00 | 65.00 | |
| ATOM | 1986 | OD1 | ASP | | 416 | −40.169 | 115.141 | −65.488 | 1.00 | 66.07 | |
| ATOM | 1987 | OD2 | ASP | | 416 | −38.737 | 116.824 | −65.521 | 1.00 | 66.23 | |
| ATOM | 1988 | C | ASP | | 416 | −37.074 | 112.499 | −64.366 | 1.00 | 53.11 | |
| ATOM | 1989 | O | ASP | | 416 | −37.668 | 111.434 | −64.496 | 1.00 | 52.07 | |
| ATOM | 1990 | N | VAL | | 417 | −35.759 | 112.627 | −64.404 | 1.00 | 52.21 | |
| ATOM | 1991 | CA | VAL | | 417 | −34.863 | 111.501 | −64.610 | 1.00 | 51.80 | |
| ATOM | 1992 | CB | VAL | | 417 | −33.412 | 111.961 | −64.467 | 1.00 | 49.97 | |
| ATOM | 1993 | CG1 | VAL | | 417 | −32.472 | 110.762 | −64.483 | 1.00 | 51.02 | |
| ATOM | 1994 | CG2 | VAL | | 417 | −33.100 | 112.908 | −65.604 | 1.00 | 45.80 | |
| ATOM | 1995 | C | VAL | | 417 | −35.158 | 110.384 | −63.627 | 1.00 | 50.42 | |
| ATOM | 1996 | O | VAL | | 417 | −35.576 | 109.298 | −64.028 | 1.00 | 50.11 | |
| ATOM | 1997 | N | VAL | | 418 | −35.003 | 110.674 | −62.340 | 1.00 | 50.05 | |
| ATOM | 1998 | CA | VAL | | 418 | −35.252 | 109.672 | −61.320 | 1.00 | 50.83 | |
| ATOM | 1999 | CB | VAL | | 418 | −34.743 | 110.146 | −59.955 | 1.00 | 52.90 | |
| ATOM | 2000 | CG1 | VAL | | 418 | −33.496 | 110.981 | −60.158 | 1.00 | 54.38 | |
| ATOM | 2001 | CG2 | VAL | | 418 | −35.820 | 110.936 | −59.219 | 1.00 | 53.52 | |
| ATOM | 2002 | C | VAL | | 418 | −36.720 | 109.296 | −61.218 | 1.00 | 52.01 | |
| ATOM | 2003 | O | VAL | | 418 | −37.071 | 108.281 | −60.615 | 1.00 | 52.28 | |
| ATOM | 2004 | N | GLN | | 419 | −37.592 | 110.101 | −61.802 | 1.00 | 52.94 | |
| ATOM | 2005 | CA | GLN | | 419 | −39.001 | 109.765 | −61.745 | 1.00 | 55.18 | |
| ATOM | 2006 | CB | GLN | | 419 | −39.863 | 111.032 | −61.754 | 1.00 | 57.66 | |
| ATOM | 2007 | CG | GLN | | 419 | −39.825 | 111.869 | −60.460 | 1.00 | 59.36 | |
| ATOM | 2008 | CD | GLN | | 419 | −40.442 | 111.155 | −59.276 | 1.00 | 62.06 | |
| ATOM | 2009 | OE1 | GLN | | 419 | −41.601 | 110.737 | −59.321 | 1.00 | 64.62 | |
| ATOM | 2010 | NE2 | GLN | | 419 | −39.670 | 111.012 | −58.202 | 1.00 | 64.83 | |
| ATOM | 2011 | C | GLN | | 419 | −39.421 | 108.845 | −62.889 | 1.00 | 56.26 | |
| ATOM | 2012 | O | GLN | | 419 | −40.572 | 108.409 | −62.934 | 1.00 | 57.06 | |
| ATOM | 2013 | N | LYS | | 420 | −38.490 | 108.517 | −63.790 | 1.00 | 56.08 | |
| ATOM | 2014 | CA | LYS | | 420 | −38.814 | 107.657 | −64.931 | 1.00 | 55.57 | |
| ATOM | 2015 | CB | LYS | | 420 | −39.320 | 106.287 | −64.471 | 1.00 | 54.42 | |
| ATOM | 2016 | CG | LYS | | 420 | −38.255 | 105.261 | −64.104 | 1.00 | 55.72 | |
| ATOM | 2017 | CD | LYS | | 420 | −38.819 | 103.826 | −64.034 | 1.00 | 55.51 | |
| ATOM | 2018 | CE | LYS | | 420 | −37.677 | 102.805 | −63.774 | 1.00 | 59.38 | |
| ATOM | 2019 | NZ | LYS | | 420 | −38.076 | 101.362 | −63.591 | 1.00 | 58.45 | |
| ATOM | 2020 | C | LYS | | 420 | −39.926 | 108.356 | −65.724 | 1.00 | 57.42 | |
| ATOM | 2021 | O | LYS | | 420 | −40.726 | 107.716 | −66.424 | 1.00 | 56.09 | |
| ATOM | 2022 | N | LYS | | 421 | −39.971 | 109.678 | −65.585 | 1.00 | 58.45 | |
| ATOM | 2023 | CA | LYS | | 421 | −40.969 | 110.499 | −66.243 | 1.00 | 60.43 | |
| ATOM | 2024 | CB | LYS | | 421 | −41.145 | 111.808 | −65.463 | 1.00 | 63.06 | |
| ATOM | 2025 | CG | LYS | | 421 | −42.569 | 112.092 | −65.016 | 1.00 | 66.83 | |
| ATOM | 2026 | CD | LYS | | 421 | −43.152 | 110.957 | −64.175 | 1.00 | 68.90 | |
| ATOM | 2027 | CE | LYS | | 421 | −44.627 | 111.224 | −63.855 | 1.00 | 71.68 | |
| ATOM | 2028 | NZ | LYS | | 421 | −44.839 | 112.504 | −63.115 | 1.00 | 71.58 | |
| ATOM | 2029 | C | LYS | | 421 | −40.562 | 110.792 | −67.689 | 1.00 | 61.55 | |
| ATOM | 2030 | O | LYS | | 421 | −41.394 | 111.164 | −68.516 | 1.00 | 61.24 | |
| ATOM | 2031 | N | LEU | | 422 | −39.279 | 110.625 | −67.992 | 1.00 | 61.71 | |
| ATOM | 2032 | CA | LEU | | 422 | −38.795 | 110.870 | −69.343 | 1.00 | 61.12 | |
| ATOM | 2033 | CB | LEU | | 422 | −37.273 | 111.024 | −69.358 | 1.00 | 58.69 | |
| ATOM | 2034 | CG | LEU | | 422 | −36.793 | 112.227 | −68.551 | 1.00 | 57.09 | |
| ATOM | 2035 | CD1 | LEU | | 422 | −35.328 | 112.468 | −68.787 | 1.00 | 55.92 | |
| ATOM | 2036 | CD2 | LEU | | 422 | −37.591 | 113.449 | −68.962 | 1.00 | 58.70 | |
| ATOM | 2037 | C | LEU | | 422 | −39.197 | 109.703 | −70.216 | 1.00 | 61.35 | |
| ATOM | 2038 | O | LEU | | 422 | −39.262 | 108.575 | −69.748 | 1.00 | 62.11 | |
| ATOM | 2039 | N | LEU | | 423 | −39.491 | 109.977 | −71.481 | 1.00 | 62.29 | |
| ATOM | 2040 | CA | LEU | | 423 | −39.868 | 108.920 | −72.410 | 1.00 | 62.77 | |
| ATOM | 2041 | CB | LEU | | 423 | −40.817 | 109.480 | −73.481 | 1.00 | 62.00 | |
| ATOM | 2042 | CG | LEU | | 423 | −41.542 | 108.475 | −74.382 | 1.00 | 63.36 | |
| ATOM | 2043 | CD1 | LEU | | 423 | −42.505 | 107.628 | −73.551 | 1.00 | 61.18 | |
| ATOM | 2044 | CD2 | LEU | | 423 | −42.289 | 109.224 | −75.483 | 1.00 | 63.05 | |
| ATOM | 2045 | C | LEU | | 423 | −38.584 | 108.341 | −73.044 | 1.00 | 61.73 | |
| ATOM | 2046 | O | LEU | | 423 | −37.677 | 109.066 | −73.452 | 1.00 | 61.55 | |
| ATOM | 2047 | N | PRO | | 424 | −38.476 | 107.018 | −73.097 | 1.00 | 62.47 | |
| ATOM | 2048 | CD | PRO | | 424 | −39.311 | 105.961 | −72.495 | 1.00 | 63.37 | |
| ATOM | 2049 | CA | PRO | | 424 | −37.255 | 106.472 | −73.700 | 1.00 | 62.30 | |
| ATOM | 2050 | CB | PRO | | 424 | −37.243 | 105.029 | −73.198 | 1.00 | 62.87 | |
| ATOM | 2051 | CG | PRO | | 424 | −38.707 | 104.694 | −73.079 | 1.00 | 62.90 | |
| ATOM | 2052 | C | PRO | | 424 | −37.251 | 106.575 | −75.231 | 1.00 | 61.64 | |
| ATOM | 2053 | O | PRO | | 424 | −38.209 | 106.163 | −75.897 | 1.00 | 61.35 | |
| ATOM | 2054 | N | PRO | | 425 | −36.167 | 107.126 | −75.806 | 1.00 | 60.32 | |
| ATOM | 2055 | CD | PRO | | 425 | −34.925 | 107.498 | −75.121 | 1.00 | 59.42 | |
| ATOM | 2056 | CA | PRO | | 425 | −36.004 | 107.298 | −77.251 | 1.00 | 60.15 | |
| ATOM | 2057 | CB | PRO | | 425 | −34.623 | 107.916 | −77.368 | 1.00 | 59.16 | |
| ATOM | 2058 | CG | PRO | | 425 | −33.924 | 107.335 | −76.226 | 1.00 | 59.65 | |
| ATOM | 2059 | C | PRO | | 425 | −36.118 | 106.001 | −78.040 | 1.00 | 60.95 | |
| ATOM | 2060 | O | PRO | | 425 | −35.993 | 106.002 | −79.269 | 1.00 | 61.28 | |
| ATOM | 2061 | N | PHE | | 426 | −36.359 | 104.902 | −77.332 | 1.00 | 59.85 | |
| ATOM | 2062 | CA | PHE | | 426 | −36.504 | 103.604 | −77.964 | 1.00 | 59.45 | |
| ATOM | 2063 | CB | PHE | | 426 | −35.149 | 103.014 | −78.319 | 1.00 | 60.30 | |
| ATOM | 2064 | CG | PHE | | 426 | −35.213 | 101.553 | −78.647 | 1.00 | 61.86 | |
| ATOM | 2065 | CD1 | PHE | | 426 | −35.996 | 101.100 | −79.699 | 1.00 | 62.98 | |
| ATOM | 2066 | CD2 | PHE | | 426 | −34.518 | 100.622 | −77.886 | 1.00 | 61.05 | |
| ATOM | 2067 | CE1 | PHE | | 426 | −36.082 | 99.738 | −79.984 | 1.00 | 63.40 | |
| ATOM | 2068 | CE2 | PHE | | 426 | −34.598 | 99.269 | −78.163 | 1.00 | 60.51 | |
| ATOM | 2069 | CZ | PHE | | 426 | −35.380 | 98.824 | −79.211 | 1.00 | 61.73 | |
| ATOM | 2070 | C | PHE | | 426 | −37.231 | 102.613 | −77.089 | 1.00 | 59.96 | |
| ATOM | 2071 | O | PHE | | 426 | −36.870 | 102.400 | −75.936 | 1.00 | 60.41 | |
| ATOM | 2072 | N | LYS | | 427 | −38.248 | 101.978 | −77.635 | 1.00 | 60.95 | |
| ATOM | 2073 | CA | LYS | | 427 | −38.956 | 101.019 | −76.834 | 1.00 | 64.11 | |
| ATOM | 2074 | CB | LYS | | 427 | −40.449 | 101.315 | −76.817 | 1.00 | 66.80 | |
| ATOM | 2075 | CG | LYS | | 427 | −41.223 | 100.379 | −75.906 | 1.00 | 69.63 | |
| ATOM | 2076 | CD | LYS | | 427 | −42.583 | 100.941 | −75.606 | 1.00 | 73.24 | |
| ATOM | 2077 | CE | LYS | | 427 | −43.325 | 100.103 | −74.583 | 1.00 | 75.62 | |
| ATOM | 2078 | NZ | LYS | | 427 | −44.685 | 100.695 | −74.329 | 1.00 | 78.61 | |
| ATOM | 2079 | C | LYS | | 427 | −38.718 | 99.619 | −77.334 | 1.00 | 65.11 | |
| ATOM | 2080 | O | LYS | | 427 | −38.968 | 99.311 | −78.490 | 1.00 | 63.93 | |
| ATOM | 2081 | N | PRO | | 428 | −38.203 | 98.752 | −76.460 | 1.00 | 67.83 | |
| ATOM | 2082 | CD | PRO | | 428 | −37.755 | 99.068 | −75.095 | 1.00 | 67.46 | |
| ATOM | 2083 | CA | PRO | | 428 | −37.920 | 97.354 | −76.796 | 1.00 | 69.61 | |
| ATOM | 2084 | CB | PRO | | 428 | −37.394 | 96.793 | −75.484 | 1.00 | 69.12 | |
| ATOM | 2085 | CG | PRO | | 428 | −36.726 | 98.003 | −74.859 | 1.00 | 68.49 | |
| ATOM | 2086 | C | PRO | | 428 | −39.230 | 96.708 | −77.235 | 1.00 | 72.00 | |
| ATOM | 2087 | O | PRO | | 428 | −40.306 | 97.111 | −76.779 | 1.00 | 71.52 | |
| ATOM | 2088 | N | GLN | | 429 | −39.144 | 95.704 | −78.097 | 1.00 | 74.15 | |
| ATOM | 2089 | CA | GLN | | 429 | −40.343 | 95.067 | −78.613 | 1.00 | 79.21 | |
| ATOM | 2090 | CB | GLN | | 429 | −40.092 | 94.660 | −80.078 | 1.00 | 81.34 | |
| ATOM | 2091 | CG | GLN | | 429 | −39.762 | 95.862 | −80.998 | 1.00 | 83.36 | |
| ATOM | 2092 | CD | GLN | | 429 | −38.427 | 95.726 | −81.758 | 1.00 | 84.49 | |
| ATOM | 2093 | OE1 | GLN | | 429 | −37.441 | 95.201 | −81.233 | 1.00 | 84.83 | |
| ATOM | 2094 | NE2 | GLN | | 429 | −38.394 | 96.229 | −82.990 | 1.00 | 84.60 | |
| ATOM | 2095 | C | GLN | | 429 | −40.900 | 93.890 | −77.799 | 1.00 | 81.25 | |
| ATOM | 2096 | O | GLN | | 429 | −41.217 | 92.841 | −78.358 | 1.00 | 81.88 | |
| ATOM | 2097 | N | VAL | | 430 | −41.049 | 94.076 | −76.487 | 1.00 | 83.33 | |
| ATOM | 2098 | CA | VAL | | 430 | −41.575 | 93.022 | −75.607 | 1.00 | 84.98 | |
| ATOM | 2099 | CB | VAL | | 430 | −40.690 | 92.858 | −74.342 | 1.00 | 83.81 | |
| ATOM | 2100 | CG1 | VAL | | 430 | −39.398 | 92.181 | −74.706 | 1.00 | 82.55 | |
| ATOM | 2101 | CG2 | VAL | | 430 | −40.395 | 94.213 | −73.727 | 1.00 | 82.81 | |
| ATOM | 2102 | C | VAL | | 430 | −43.031 | 93.269 | −75.168 | 1.00 | 87.28 | |
| ATOM | 2103 | O | VAL | | 430 | −43.430 | 94.416 | −74.927 | 1.00 | 88.31 | |
| ATOM | 2104 | N | THR | | 431 | −43.819 | 92.196 | −75.068 | 1.00 | 88.25 | |
| ATOM | 2105 | CA | THR | | 431 | −45.232 | 92.300 | −74.667 | 1.00 | 89.29 | |
| ATOM | 2106 | CB | THR | | 431 | −45.928 | 90.908 | −74.636 | 1.00 | 89.30 | |
| ATOM | 2107 | OG1 | THR | | 431 | −45.791 | 90.259 | −75.908 | 1.00 | 90.23 | |
| ATOM | 2108 | CG2 | THR | | 431 | −47.406 | 91.060 | −74.306 | 1.00 | 89.06 | |
| ATOM | 2109 | C | THR | | 431 | −45.340 | 92.883 | −73.263 | 1.00 | 89.57 | |
| ATOM | 2110 | O | THR | | 431 | −45.818 | 93.998 | −73.046 | 1.00 | 88.88 | |
| ATOM | 2111 | N | SER | | 432 | −44.890 | 92.085 | −72.311 | 1.00 | 90.05 | |
| ATOM | 2112 | CA | SER | | 432 | −44.896 | 92.452 | −70.916 | 1.00 | 90.57 | |
| ATOM | 2113 | CB | SER | | 432 | −46.073 | 91.781 | −70.221 | 1.00 | 91.71 | |
| ATOM | 2114 | OG | SER | | 432 | −46.052 | 90.383 | −70.467 | 1.00 | 92.70 | |
| ATOM | 2115 | C | SER | | 432 | −43.590 | 91.893 | −70.386 | 1.00 | 90.68 | |
| ATOM | 2116 | O | SER | | 432 | −42.878 | 91.192 | −71.111 | 1.00 | 90.77 | |
| ATOM | 2117 | N | GLU | | 433 | −43.266 | 92.193 | −69.134 | 1.00 | 90.44 | |
| ATOM | 2118 | CA | GLU | | 433 | −42.035 | 91.677 | −68.568 | 1.00 | 90.51 | |
| ATOM | 2119 | CB | GLU | | 433 | −41.873 | 92.168 | −67.129 | 1.00 | 91.44 | |
| ATOM | 2120 | CG | GLU | | 433 | −43.165 | 92.298 | −66.370 | 1.00 | 93.97 | |
| ATOM | 2121 | CD | GLU | | 433 | −43.077 | 93.334 | −65.263 | 1.00 | 95.97 | |
| ATOM | 2122 | OE1 | GLU | | 433 | −42.108 | 93.279 | −64.472 | 1.00 | 95.47 | |
| ATOM | 2123 | OE2 | GLU | | 433 | −43.981 | 94.204 | −65.188 | 1.00 | 97.07 | |
| ATOM | 2124 | C | GLU | | 433 | −42.003 | 90.150 | −68.651 | 1.00 | 89.80 | |
| ATOM | 2125 | O | GLU | | 433 | −40.965 | 89.529 | −68.447 | 1.00 | 89.98 | |
| ATOM | 2126 | N | VAL | | 434 | −43.145 | 89.561 | −68.989 | 1.00 | 89.02 | |
| ATOM | 2127 | CA | VAL | | 434 | −43.276 | 88.114 | −69.122 | 1.00 | 87.58 | |
| ATOM | 2128 | CB | VAL | | 434 | −44.762 | 87.677 | −69.051 | 1.00 | 87.60 | |
| ATOM | 2129 | CG1 | VAL | | 434 | −44.863 | 86.181 | −68.774 | 1.00 | 86.18 | |
| ATOM | 2130 | CG2 | VAL | | 434 | −45.495 | 88.484 | −67.990 | 1.00 | 87.26 | |
| ATOM | 2131 | C | VAL | | 434 | −42.724 | 87.701 | −70.476 | 1.00 | 86.78 | |
| ATOM | 2132 | O | VAL | | 434 | −42.287 | 86.567 | −70.664 | 1.00 | 86.37 | |
| ATOM | 2133 | N | ASP | | 435 | −42.756 | 88.632 | −71.422 | 1.00 | 86.52 | |
| ATOM | 2134 | CA | ASP | | 435 | −42.257 | 88.362 | −72.758 | 1.00 | 86.28 | |
| ATOM | 2135 | CB | ASP | | 435 | −42.355 | 89.613 | −73.629 | 1.00 | 86.61 | |
| ATOM | 2136 | CG | ASP | | 435 | −41.944 | 89.350 | −75.062 | 1.00 | 87.61 | |
| ATOM | 2137 | OD1 | ASP | | 435 | −42.491 | 88.411 | −75.679 | 1.00 | 87.73 | |
| ATOM | 2138 | OD2 | ASP | | 435 | −41.070 | 90.078 | −75.574 | 1.00 | 88.49 | |
| ATOM | 2139 | C | ASP | | 435 | −40.809 | 87.892 | −72.676 | 1.00 | 85.62 | |
| ATOM | 2140 | O | ASP | | 435 | −39.937 | 88.611 | −72.180 | 1.00 | 84.77 | |
| ATOM | 2141 | N | THR | | 436 | −40.562 | 86.686 | −73.178 | 1.00 | 85.48 | |
| ATOM | 2142 | CA | THR | | 436 | −39.229 | 86.085 | −73.138 | 1.00 | 85.33 | |
| ATOM | 2143 | CB | THR | | 436 | −39.323 | 84.597 | −72.743 | 1.00 | 84.60 | |
| ATOM | 2144 | OG1 | THR | | 436 | −39.815 | 83.843 | −73.857 | 1.00 | 83.80 | |
| ATOM | 2145 | CG2 | THR | | 436 | −40.270 | 84.417 | −71.566 | 1.00 | 82.29 | |
| ATOM | 2146 | C | THR | | 436 | −38.455 | 86.160 | −74.455 | 1.00 | 85.27 | |
| ATOM | 2147 | O | THR | | 436 | −37.513 | 85.390 | −74.669 | 1.00 | 84.76 | |
| ATOM | 2148 | N | ARG | | 437 | −38.820 | 87.092 | −75.326 | 1.00 | 85.80 | |
| ATOM | 2149 | CA | ARG | | 437 | −38.146 | 87.170 | −76.613 | 1.00 | 86.96 | |
| ATOM | 2150 | CB | ARG | | 437 | −39.005 | 87.931 | −77.626 | 1.00 | 87.46 | |
| ATOM | 2151 | CG | ARG | | 437 | −38.985 | 89.432 | −77.509 | 1.00 | 88.62 | |
| ATOM | 2152 | CD | ARG | | 437 | −39.638 | 90.034 | −78.749 | 1.00 | 90.99 | |
| ATOM | 2153 | NE | ARG | | 437 | −40.987 | 89.502 | −78.970 | 1.00 | 92.25 | |
| ATOM | 2154 | CZ | ARG | | 437 | −41.689 | 89.666 | −80.090 | 1.00 | 92.57 | |
| ATOM | 2155 | NH1 | ARG | | 437 | −41.181 | 90.351 | −81.109 | 1.00 | 93.15 | |
| ATOM | 2156 | NH2 | ARG | | 437 | −42.901 | 89.138 | −80.195 | 1.00 | 92.65 | |
| ATOM | 2157 | C | ARG | | 437 | −36.712 | 87.695 | −76.670 | 1.00 | 86.99 | |
| ATOM | 2158 | O | ARG | | 437 | −36.017 | 87.418 | −77.643 | 1.00 | 87.72 | |
| ATOM | 2159 | N | TYR | | 438 | −36.249 | 88.442 | −75.669 | 1.00 | 86.84 | |
| ATOM | 2160 | CA | TYR | | 438 | −34.868 | 88.928 | −75.713 | 1.00 | 86.83 | |
| ATOM | 2161 | CB | TYR | | 438 | −34.727 | 90.299 | −75.042 | 1.00 | 86.68 | |
| ATOM | 2162 | CG | TYR | | 438 | −35.112 | 91.450 | −75.945 | 1.00 | 87.90 | |
| ATOM | 2163 | CD1 | TYR | | 438 | −36.343 | 92.097 | −75.803 | 1.00 | 87.81 | |
| ATOM | 2164 | CE1 | TYR | | 438 | −36.723 | 93.135 | −76.663 | 1.00 | 87.32 | |
| ATOM | 2165 | CD2 | TYR | | 438 | −34.262 | 91.872 | −76.973 | 1.00 | 87.74 | |
| ATOM | 2166 | CE2 | TYR | | 438 | −34.629 | 92.910 | −77.841 | 1.00 | 86.99 | |
| ATOM | 2167 | CZ | TYR | | 438 | −35.863 | 93.534 | −77.679 | 1.00 | 87.49 | |
| ATOM | 2168 | OH | TYR | | 438 | −36.244 | 94.545 | −78.536 | 1.00 | 87.87 | |
| ATOM | 2169 | C | TYR | | 438 | −33.903 | 87.951 | −75.064 | 1.00 | 86.82 | |
| ATOM | 2170 | O | TYR | | 438 | −32.692 | 88.165 | −75.066 | 1.00 | 87.00 | |
| ATOM | 2171 | N | PHE | | 439 | −34.441 | 86.864 | −74.528 | 1.00 | 87.29 | |
| ATOM | 2172 | CA | PHE | | 439 | −33.620 | 85.865 | −73.863 | 1.00 | 88.45 | |
| ATOM | 2173 | CB | PHE | | 439 | −34.498 | 85.037 | −72.920 | 1.00 | 86.80 | |
| ATOM | 2174 | CG | PHE | | 439 | −35.133 | 85.846 | −71.813 | 1.00 | 85.58 | |
| ATOM | 2175 | CD1 | PHE | | 439 | −36.068 | 85.270 | −70.959 | 1.00 | 85.32 | |
| ATOM | 2176 | CD2 | PHE | | 439 | −34.803 | 87.189 | −71.628 | 1.00 | 85.46 | |
| ATOM | 2177 | CE1 | PHE | | 439 | −36.664 | 86.015 | −69.940 | 1.00 | 85.22 | |
| ATOM | 2178 | CE2 | PHE | | 439 | −35.393 | 87.940 | −70.615 | 1.00 | 85.05 | |
| ATOM | 2179 | CZ | PHE | | 439 | −36.325 | 87.354 | −69.769 | 1.00 | 85.55 | |
| ATOM | 2180 | C | PHE | | 439 | −32.841 | 84.958 | −74.817 | 1.00 | 89.81 | |
| ATOM | 2181 | O | PHE | | 439 | −32.724 | 83.761 | −74.581 | 1.00 | 90.39 | |
| ATOM | 2182 | N | ASP | | 440 | −32.308 | 85.549 | −75.888 | 1.00 | 92.18 | |
| ATOM | 2183 | CA | ASP | | 440 | −31.505 | 84.851 | −76.906 | 1.00 | 93.77 | |
| ATOM | 2184 | CB | ASP | | 440 | −32.378 | 84.402 | −78.085 | 1.00 | 94.49 | |
| ATOM | 2185 | CG | ASP | | 440 | −33.503 | 83.476 | −77.665 | 1.00 | 95.46 | |
| ATOM | 2186 | OD1 | ASP | | 440 | −34.347 | 83.889 | −76.838 | 1.00 | 95.57 | |
| ATOM | 2187 | OD2 | ASP | | 440 | −33.549 | 82.335 | −78.170 | 1.00 | 95.83 | |
| ATOM | 2188 | C | ASP | | 440 | −30.462 | 85.858 | −77.414 | 1.00 | 94.78 | |
| ATOM | 2189 | O | ASP | | 440 | −30.043 | 86.747 | −76.657 | 1.00 | 95.43 | |
| ATOM | 2190 | N | ASP | | 441 | −30.051 | 85.729 | −78.680 | 1.00 | 95.03 | |
| ATOM | 2191 | CA | ASP | | 441 | −29.075 | 86.656 | −79.279 | 1.00 | 95.25 | |
| ATOM | 2192 | CB | ASP | | 441 | −27.915 | 85.901 | −79.960 | 1.00 | 95.91 | |
| ATOM | 2193 | CG | ASP | | 441 | −26.870 | 85.376 | −78.966 | 1.00 | 97.03 | |
| ATOM | 2194 | OD1 | ASP | | 441 | −27.176 | 84.396 | −78.239 | 1.00 | 97.59 | |
| ATOM | 2195 | OD2 | ASP | | 441 | −25.744 | 85.945 | −78.915 | 1.00 | 95.54 | |
| ATOM | 2196 | C | ASP | | 441 | −29.749 | 87.569 | −80.310 | 1.00 | 95.02 | |
| ATOM | 2197 | O | ASP | | 441 | −29.416 | 87.547 | −81.498 | 1.00 | 94.55 | |
| ATOM | 2198 | OH2 | TIP | | 1 | −17.565 | 102.189 | −62.350 | 1.00 | 37.43 | |
| ATOM | 2199 | OH2 | TIP | | 2 | −33.587 | 91.116 | −71.760 | 1.00 | 41.39 | |
| ATOM | 2200 | OH2 | TIP | | 3 | −17.875 | 100.298 | −70.868 | 1.00 | 46.03 | |
| ATOM | 2201 | OH2 | TIP | | 4 | −25.044 | 95.430 | −72.610 | 1.00 | 42.52 | |
| ATOM | 2202 | OH2 | TIP | | 5 | −23.822 | 111.358 | −54.564 | 1.00 | 45.34 | |
| ATOM | 2203 | OH2 | TIP | | 6 | −39.172 | 106.198 | −60.025 | 1.00 | 45.47 | |
| ATOM | 2204 | OH2 | TIP | | 7 | −17.966 | 101.207 | −52.051 | 1.00 | 55.19 | |
| ATOM | 2205 | OH2 | TIP | | 8 | −14.825 | 102.146 | −63.563 | 1.00 | 36.22 | |
| ATOM | 2206 | OH2 | TIP | | 9 | −12.646 | 109.426 | −56.476 | 1.00 | 39.70 | |
| ATOM | 2207 | OH2 | TIP | | 10 | −26.220 | 112.515 | −82.519 | 1.00 | 54.10 | |
| ATOM | 2208 | OH2 | TIP | | 11 | −21.056 | 96.909 | −58.004 | 1.00 | 50.75 | |
| ATOM | 2209 | OH2 | TIP | | 12 | −12.363 | 100.866 | −62.927 | 1.00 | 41.26 | |
| ATOM | 2210 | OH2 | TIP | | 13 | −1.321 | 111.343 | −71.328 | 1.00 | 80.10 | |
| ATOM | 2211 | OH2 | TIP | | 14 | −15.955 | 100.816 | −65.204 | 1.00 | 40.00 | |
| ATOM | 2212 | OH2 | TIP | | 15 | −38.264 | 94.351 | −68.026 | 1.00 | 56.00 | |
| ATOM | 2213 | OH2 | TIP | | 16 | −34.747 | 103.092 | −74.730 | 1.00 | 54.31 | |
| ATOM | 2214 | OH2 | TIP | | 17 | −36.658 | 81.314 | −74.149 | 1.00 | 71.15 | |
| ATOM | 2215 | OH2 | TIP | | 18 | −16.949 | 98.679 | −85.728 | 1.00 | 87.62 | |
| ATOM | 2216 | OH2 | TIP | | 21 | −18.846 | 109.431 | −51.863 | 1.00 | 51.80 | |
| ATOM | 2217 | OH2 | TIP | | 22 | −19.808 | 89.557 | −73.154 | 1.00 | 50.02 | |
| ATOM | 2218 | OH2 | TIP | | 23 | −15.508 | 95.436 | −70.540 | 1.00 | 88.56 | |
| ATOM | 2219 | OH2 | TIP | | 24 | −14.481 | 98.173 | −68.748 | 1.00 | 55.20 | |
| ATOM | 2220 | OH2 | TIP | | 25 | −12.701 | 111.957 | −87.032 | 1.00 | 60.67 | |
| ATOM | 2221 | OH2 | TIP | | 26 | −16.126 | 117.347 | −81.325 | 1.00 | 92.19 | |
| ATOM | 2222 | OH2 | TIP | | 28 | −32.498 | 88.797 | −82.315 | 1.00 | 63.65 | |
| ATOM | 2223 | OH2 | TIP | | 29 | −27.830 | 77.286 | −70.926 | 1.00 | 64.26 | |
| ATOM | 2224 | OH2 | TIP | | 30 | −18.104 | 75.804 | −72.803 | 1.00 | 62.36 | |
| ATOM | 2225 | OH2 | TIP | | 31 | −4.498 | 114.218 | −69.024 | 1.00 | 50.34 | |
| ATOM | 2226 | OH2 | TIP | | 32 | −27.549 | 116.364 | −75.607 | 1.00 | 38.10 | |
| ATOM | 2227 | OH2 | TIP | | 33 | −20.441 | 122.193 | −68.960 | 1.00 | 57.93 | |
| ATOM | 2228 | OH2 | TIP | | 34 | −38.352 | 98.016 | −64.877 | 1.00 | 48.98 | |
| ATOM | 2229 | OH2 | TIP | | 35 | −25.169 | 109.804 | −72.683 | 1.00 | 62.00 | |
| ATOM | 2230 | OH2 | TIP | | 36 | −15.952 | 120.584 | −76.396 | 1.00 | 60.49 | |
| ATOM | 2231 | OH2 | TIP | | 37 | −31.772 | 109.042 | −81.185 | 1.00 | 61.74 | |
| ATOM | 2232 | OH2 | TIP | | 38 | −26.023 | 117.180 | −77.997 | 1.00 | 45.99 | |
| ATOM | 2233 | OH2 | TIP | | 39 | −30.417 | 119.656 | −66.158 | 1.00 | 57.36 | |
| ATOM | 2234 | OH2 | TIP | | 40 | −31.769 | 114.324 | −58.313 | 1.00 | 54.48 | |
| ATOM | 2235 | OH2 | TIP | | 41 | −21.640 | 114.988 | −52.575 | 1.00 | 59.03 | |
| ATOM | 2236 | OH2 | TIP | | 42 | −46.473 | 91.759 | −56.427 | 1.00 | 61.38 | |
| ATOM | 2237 | OH2 | TIP | | 43 | −42.689 | 114.142 | −63.306 | 1.00 | 81.90 | |
| ATOM | 2238 | OH2 | TIP | | 44 | −14.015 | 126.468 | −54.963 | 1.00 | 61.90 | |
| ATOM | 2239 | OH2 | TIP | | 45 | −27.585 | 121.073 | −60.489 | 1.00 | 62.27 | |
| ATOM | 2240 | OH2 | TIP | | 46 | −16.865 | 101.333 | −68.880 | 1.00 | 55.82 | |
| ATOM | 2241 | OH2 | TIP | | 47 | −34.735 | 117.310 | −60.322 | 1.00 | 60.49 | |
| ATOM | 2242 | OH2 | TIP | | 48 | −9.234 | 93.074 | −70.733 | 1.00 | 72.22 | |
| ATOM | 2243 | OH2 | TIP | | 49 | −40.289 | 91.726 | −57.862 | 1.00 | 50.18 | |
| ATOM | 2244 | OH2 | TIP | | 50 | −9.221 | 112.888 | −55.496 | 1.00 | 54.20 | |
| ATOM | 2245 | OH2 | TIP | | 51 | −38.906 | 112.515 | −66.710 | 1.00 | 78.66 | |
| ATOM | 2246 | OH2 | TIP | | 52 | 2.529 | 96.809 | −74.765 | 1.00 | 69.23 | |
| ATOM | 2247 | OH2 | TIP | | 53 | −24.199 | 72.889 | −78.644 | 1.00 | 59.85 | |
| ATOM | 2248 | OH2 | TIP | | 54 | −31.754 | 89.026 | −72.509 | 1.00 | 62.94 | |
| ATOM | 2249 | OH2 | TIP | | 55 | −20.971 | 74.991 | −59.317 | 1.00 | 61.24 | |
| ATOM | 2250 | OH2 | TIP | | 56 | −26.546 | 125.032 | −70.146 | 1.00 | 62.26 | |
| ATOM | 2251 | OH2 | TIP | | 57 | −47.392 | 111.329 | −65.455 | 1.00 | 63.14 | |
| ATOM | 2252 | OH2 | TIP | | 58 | −32.406 | 116.433 | −73.716 | 1.00 | 52.36 | |
| ATOM | 2253 | OH2 | TIP | | 59 | −23.830 | 94.667 | −52.040 | 1.00 | 75.89 | |
| ATOM | 2254 | OH2 | TIP | | 60 | −30.049 | 91.062 | −63.135 | 1.00 | 59.19 | |
| ATOM | 2255 | OH2 | TIP | | 61 | −20.767 | 90.050 | −71.065 | 1.00 | 61.77 | |
| ATOM | 2256 | OH2 | TIP | | 62 | −14.383 | 86.763 | −62.584 | 1.00 | 66.17 | |
| ATOM | 2257 | OH2 | TIP | | 63 | −24.503 | 106.816 | −84.818 | 1.00 | 46.19 | |
| ATOM | 2258 | OH2 | TIP | | 64 | −47.457 | 95.958 | −60.114 | 1.00 | 64.05 | |
| ATOM | 2259 | OH2 | TIP | | 65 | −33.015 | 107.500 | −54.598 | 1.00 | 57.09 | |
| ATOM | 2260 | OH2 | TIP | | 66 | −19.177 | 72.293 | −58.651 | 1.00 | 65.08 | |
| ATOM | 2261 | OH2 | TIP | | 67 | −42.245 | 87.048 | −85.668 | 1.00 | 80.22 | |
| ATOM | 2262 | OH2 | TIP | | 68 | −41.110 | 105.176 | −75.745 | 1.00 | 55.61 | |
| ATOM | 2263 | OH2 | TIP | | 69 | −17.900 | 101.391 | −73.416 | 1.00 | 50.26 | |
| ATOM | 2264 | OH2 | TIP | | 70 | −15.112 | 122.752 | −72.280 | 1.00 | 62.23 | |
| ATOM | 2265 | OH2 | TIP | | 71 | −10.897 | 110.554 | −88.353 | 1.00 | 59.35 | |
| ATOM | 2266 | OH2 | TIP | | 72 | −15.863 | 87.894 | −40.536 | 1.00 | 76.19 | |
| ATOM | 2267 | OH2 | TIP | | 73 | −13.269 | 89.222 | −73.978 | 1.00 | 72.37 | |
| ATOM | 2268 | OH2 | TIP | | 74 | −12.819 | 125.509 | −83.766 | 1.00 | 61.65 | |
| ATOM | 2269 | OH2 | TIP | | 75 | −17.556 | 97.431 | −73.936 | 1.00 | 45.20 | |
| ATOM | 2270 | OH2 | TIP | | 76 | 2.814 | 106.071 | −59.216 | 1.00 | 62.17 | |
| ATOM | 2271 | OH2 | TIP | | 77 | −20.096 | 74.736 | −45.657 | 1.00 | 56.01 | |
| ATOM | 2272 | OH2 | TIP | | 78 | −12.837 | 81.660 | −39.897 | 1.00 | 61.24 | |
| ATOM | 2273 | OH2 | TIP | | 79 | −6.887 | 83.385 | −69.442 | 1.00 | 83.46 | |
| ATOM | 2274 | OH2 | TIP | | 80 | −40.679 | 99.898 | −84.715 | 1.00 | 69.12 | |
| ATOM | 2275 | OH2 | TIP | | 81 | −17.993 | 123.292 | −72.716 | 1.00 | 53.42 | |
| ATOM | 2276 | OH2 | TIP | | 82 | −46.988 | 106.360 | −64.908 | 1.00 | 60.82 | |
| ATOM | 2277 | OH2 | TIP | | 83 | −6.977 | 83.446 | −75.179 | 1.00 | 64.46 | |
| ATOM | 2278 | OH2 | TIP | | 84 | −15.030 | 112.332 | −88.404 | 1.00 | 68.15 | |
| ATOM | 2279 | OH2 | TIP | | 85 | −40.535 | 89.123 | −65.466 | 1.00 | 66.73 | |
| ATOM | 2280 | OH2 | TIP | | 86 | −23.943 | 109.923 | −81.328 | 1.00 | 53.47 | |
| ATOM | 2281 | OH2 | TIP | | 87 | −28.039 | 92.419 | −61.573 | 1.00 | 60.22 | |
| ATOM | 2282 | OH2 | TIP | | 88 | −13.595 | 91.555 | −61.879 | 1.00 | 65.84 | |
| ATOM | 2283 | OH2 | TIP | | 89 | −16.436 | 81.809 | −39.142 | 1.00 | 61.00 | |
| ATOM | 2284 | OH2 | TIP | | 90 | −7.960 | 78.226 | −49.496 | 1.00 | 56.06 | |
| ATOM | 2285 | OH2 | TIP | | 91 | −27.922 | 85.717 | −46.157 | 1.00 | 63.49 | |
| ATOM | 2286 | OH2 | TIP | | 92 | −25.252 | 85.080 | −70.790 | 1.00 | 87.29 | |
| ATOM | 2287 | OH2 | TIP | | 93 | −44.520 | 99.048 | −63.477 | 1.00 | 66.02 | |
| ATOM | 2288 | OH2 | TIP | | 94 | −14.400 | 81.735 | −70.409 | 1.00 | 58.67 | |
| ATOM | 2289 | OH2 | TIP | | 95 | −22.737 | 126.809 | −60.364 | 1.00 | 68.49 | |
| ATOM | 2290 | OH2 | TIP | | 96 | −36.302 | 116.540 | −69.002 | 1.00 | 67.72 | |
| ATOM | 2291 | OH2 | TIP | | 97 | −9.993 | 98.758 | −77.341 | 1.00 | 60.73 | |
| ATOM | 2292 | OH2 | TIP | | 98 | −5.198 | 107.622 | −56.586 | 1.00 | 62.27 | |
| ATOM | 2293 | OH2 | TIP | | 99 | −42.156 | 87.741 | −57.066 | 1.00 | 66.15 | |
| ATOM | 2294 | OH2 | TIP | | 100 | −24.999 | 97.737 | −53.947 | 1.00 | 67.24 | |
| ATOM | 2295 | OH2 | TIP | | 101 | −34.330 | 85.565 | −83.609 | 1.00 | 107.86 | |
| ATOM | 2296 | OH2 | TIP | | 102 | −46.299 | 88.604 | −57.149 | 1.00 | 63.80 | |
| ATOM | 2297 | OH2 | TIP | | 103 | −41.265 | 81.337 | −55.536 | 1.00 | 56.91 | |
| ATOM | 2298 | OH2 | TIP | | 104 | −26.510 | 109.042 | −46.918 | 1.00 | 40.04 | |
| ATOM | 2299 | OH2 | TIP | | 105 | −40.249 | 113.287 | −69.629 | 1.00 | 90.25 | |
| ATOM | 2300 | OH2 | TIP | | 106 | −8.724 | 94.018 | −77.530 | 1.00 | 56.35 | |
| ATOM | 2301 | OH2 | TIP | | 107 | −25.587 | 92.031 | −67.802 | 1.00 | 64.34 | |
| ATOM | 2302 | OH2 | TIP | | 108 | −6.596 | 117.195 | −52.572 | 1.00 | 72.27 | |
| ATOM | 2303 | OH2 | TIP | | 109 | −10.888 | 107.185 | −57.545 | 1.00 | 53.02 | |
| ATOM | 2304 | OH2 | TIP | | 110 | −0.502 | 119.304 | −51.158 | 1.00 | 61.37 | |
| ATOM | 2305 | OH2 | TIP | | 111 | −1.110 | 111.586 | −79.546 | 1.00 | 63.31 | |
| ATOM | 2306 | OH2 | TIP | | 112 | −9.865 | 114.401 | −80.681 | 1.00 | 75.67 | |
| ATOM | 2307 | OH2 | TIP | | 113 | −46.370 | 79.300 | −40.951 | 1.00 | 73.70 | |
| ATOM | 2308 | OH2 | TIP | | 114 | −29.421 | 87.726 | −46.003 | 1.00 | 69.76 | |
| ATOM | 2309 | OH2 | TIP | | 115 | −3.726 | 84.683 | −75.163 | 1.00 | 76.57 | |
| ATOM | 2310 | OH2 | TIP | | 116 | −40.377 | 85.114 | −48.505 | 1.00 | 69.00 | |
| ATOM | 2311 | OH2 | TIP | | 117 | −43.021 | 94.888 | −57.265 | 1.00 | 75.05 | |
| ATOM | 2312 | OH2 | TIP | | 118 | −26.757 | 89.010 | −44.248 | 1.00 | 71.82 | |
| ATOM | 2313 | OH2 | TIP | | 119 | −26.088 | 90.870 | −77.910 | 1.00 | 73.49 | |
| ATOM | 2314 | OH2 | TIP | | 120 | −51.720 | 94.496 | −56.733 | 1.00 | 73.77 | |
| ATOM | 2315 | OH2 | TIP | | 121 | −22.597 | 90.054 | −55.639 | 1.00 | 68.81 | |
| ATOM | 2316 | OH2 | TIP | | 122 | −48.699 | 74.961 | −56.253 | 1.00 | 74.57 | |
| ATOM | 2317 | OH2 | TIP | | 123 | −48.606 | 76.242 | −50.583 | 1.00 | 70.48 | |
| ATOM | 2318 | OH2 | TIP | | 124 | −34.958 | 81.995 | −72.529 | 1.00 | 70.39 | |
| ATOM | 2319 | OH2 | TIP | S | 1 | −24.874 | 75.096 | −52.563 | 1.00 | 100.64 | S |
| ATOM | 2320 | OH2 | TIP | S | 2 | −27.640 | 116.193 | −73.338 | 1.00 | 64.47 | S |
| ATOM | 2321 | OH2 | TIP | S | 3 | −33.593 | 103.889 | −70.517 | 1.00 | 140.05 | S |
| ATOM | 2322 | OH2 | TIP | S | 4 | −6.592 | 112.901 | −69.231 | 1.00 | 63.69 | S |
| ATOM | 2323 | OH2 | TIP | S | 5 | −29.756 | 115.038 | −73.461 | 1.00 | 97.42 | S |
| ATOM | 2324 | OH2 | TIP | S | 6 | −38.105 | 119.752 | −67.515 | 1.00 | 53.94 | S |
| ATOM | 2325 | OH2 | TIP | S | 7 | −32.981 | 119.061 | −63.624 | 1.00 | 60.23 | S |
| ATOM | 2326 | OH2 | TIP | S | 8 | −35.040 | 108.034 | −58.557 | 1.00 | 102.55 | S |
| ATOM | 2327 | OH2 | TIP | S | 9 | −31.599 | 98.109 | −46.064 | 1.00 | 83.62 | S |
| ATOM | 2328 | OH2 | TIP | S | 10 | −28.319 | 116.523 | −78.734 | 1.00 | 64.87 | S |
| ATOM | 2329 | OH2 | TIP | S | 11 | −17.321 | 112.989 | −83.893 | 1.00 | 92.65 | S |
| ATOM | 2330 | OH2 | TIP | S | 12 | −24.476 | 91.578 | −44.694 | 1.00 | 55.22 | S |
| ATOM | 2331 | OH2 | TIP | S | 13 | −29.554 | 118.607 | −68.303 | 1.00 | 61.65 | S |
| ATOM | 2332 | OH2 | TIP | S | 14 | −16.299 | 98.303 | −65.370 | 1.00 | 144.76 | S |
| ATOM | 2333 | OH2 | TIP | S | 15 | −1.478 | 95.998 | −77.354 | 1.00 | 131.79 | S |
| ATOM | 2334 | OH2 | TIP | S | 16 | −29.686 | 102.563 | −51.767 | 1.00 | 75.73 | S |
| ATOM | 2335 | OH2 | TIP | S | 17 | −36.744 | 110.842 | −72.109 | 1.00 | 118.16 | S |
| ATOM | 2336 | OH2 | TIP | S | 18 | −29.348 | 79.309 | −60.792 | 1.00 | 122.86 | S |
| ATOM | 2337 | OH2 | TIP | S | 19 | −34.282 | 105.373 | −55.136 | 1.00 | 65.27 | S |
| ATOM | 2338 | OH2 | TIP | S | 20 | −30.975 | 89.345 | −60.958 | 1.00 | 91.03 | S |
| ATOM | 2339 | OH2 | TIP | S | 21 | 2.308 | 96.056 | −77.529 | 1.00 | 85.89 | S |
| ATOM | 2340 | OH2 | TIP | S | 22 | −21.670 | 122.591 | −62.938 | 1.00 | 78.79 | S |
| ATOM | 2341 | OH2 | TIP | S | 23 | −26.498 | 116.728 | −56.181 | 1.00 | 62.33 | S |
| ATOM | 2342 | OH2 | TIP | S | 24 | −24.379 | 90.799 | −69.404 | 1.00 | 120.16 | S |
| ATOM | 2343 | OH2 | TIP | S | 25 | −15.600 | 92.352 | −50.584 | 1.00 | 63.36 | S |
| ATOM | 2344 | OH2 | TIP | S | 26 | −30.058 | 91.485 | −47.678 | 1.00 | 68.28 | S |
| ATOM | 2345 | OH2 | TIP | S | 27 | −22.559 | 96.748 | −63.089 | 1.00 | 149.69 | S |
| ATOM | 2346 | OH2 | TIP | S | 28 | −39.576 | 76.992 | −44.850 | 1.00 | 64.31 | S |
| ATOM | 2347 | OH2 | TIP | S | 29 | −34.518 | 120.744 | −73.343 | 1.00 | 119.94 | S |
| ATOM | 2348 | OH2 | TIP | S | 30 | −16.840 | 119.987 | −56.087 | 1.00 | 126.22 | S |
| ATOM | 2349 | OH2 | TIP | S | 31 | −29.914 | 106.013 | −81.685 | 1.00 | 60.80 | S |
| ATOM | 2350 | OH2 | TIP | S | 32 | −43.945 | 108.903 | −62.310 | 1.00 | 61.73 | S |
| ATOM | 2351 | OH2 | TIP | S | 33 | −4.901 | 113.071 | −67.218 | 1.00 | 86.69 | S |
| ATOM | 2352 | OH2 | TIP | S | 34 | −20.068 | 100.803 | −74.311 | 1.00 | 130.96 | S |
| ATOM | 2353 | OH2 | TIP | S | 35 | −31.212 | 112.368 | −60.484 | 1.00 | 63.96 | S |
| ATOM | 2354 | OH2 | TIP | S | 36 | −38.783 | 102.197 | −60.426 | 1.00 | 61.22 | S |
| ATOM | 2355 | OH2 | TIP | S | 37 | −26.422 | 114.665 | −76.950 | 1.00 | 142.66 | S |
| ATOM | 2356 | OH2 | TIP | S | 38 | −15.042 | 117.523 | −50.310 | 1.00 | 81.99 | S |
| ATOM | 2357 | OH2 | TIP | S | 39 | −21.749 | 124.273 | −64.985 | 1.00 | 53.70 | S |
| ATOM | 2358 | OH2 | TIP | S | 40 | −13.125 | 89.749 | −52.101 | 1.00 | 66.80 | S |
| ATOM | 2359 | OH2 | TIP | S | 41 | −18.963 | 109.880 | −54.656 | 1.00 | 121.15 | S |
| ATOM | 2360 | OH2 | TIP | S | 42 | −15.531 | 109.411 | −73.950 | 1.00 | 108.80 | S |
| ATOM | 2361 | OH2 | TIP | S | 43 | −38.932 | 116.811 | −62.960 | 1.00 | 87.34 | S |
| ATOM | 2362 | OH2 | TIP | S | 44 | −35.799 | 75.151 | −68.750 | 1.00 | 68.27 | S |
| ATOM | 2363 | OH2 | TIP | S | 45 | −14.248 | 108.177 | −52.675 | 1.00 | 57.76 | S |
| ATOM | 2364 | OH2 | TIP | S | 46 | −42.016 | 79.620 | −57.106 | 1.00 | 58.01 | S |
| ATOM | 2365 | OH2 | TIP | S | 47 | −37.376 | 89.555 | −73.417 | 1.00 | 56.77 | S |
| ATOM | 2366 | OH2 | TIP | S | 48 | −16.662 | 98.182 | −50.524 | 1.00 | 72.07 | S |
| ATOM | 2367 | OH2 | TIP | S | 49 | −31.363 | 113.752 | −80.911 | 1.00 | 115.84 | S |
| ATOM | 2368 | OH2 | TIP | S | 50 | −35.870 | 110.266 | −74.588 | 1.00 | 86.77 | S |
| ATOM | 2369 | OH2 | TIP | S | 51 | −18.880 | 88.423 | −71.504 | 1.00 | 71.73 | S |
| ATOM | 2370 | OH2 | TIP | S | 52 | −18.696 | 74.787 | −59.072 | 1.00 | 96.03 | S |
| ATOM | 2371 | OH2 | TIP | S | 53 | −1.450 | 92.204 | −72.489 | 1.00 | 82.54 | S |
| ATOM | 2372 | OH2 | TIP | S | 54 | −25.703 | 117.997 | −74.653 | 1.00 | 140.68 | S |
| ATOM | 2373 | OH2 | TIP | S | 55 | −5.538 | 113.841 | −87.337 | 1.00 | 63.97 | S |
| ATOM | 2374 | OH2 | TIP | S | 56 | −24.002 | 99.399 | −87.296 | 1.00 | 70.42 | S |
| ATOM | 2375 | OH2 | TIP | S | 57 | −35.685 | 115.383 | −71.829 | 1.00 | 59.90 | S |
| ATOM | 2376 | OH2 | TIP | S | 58 | −11.949 | 109.303 | −85.222 | 1.00 | 64.78 | S |
| ATOM | 2377 | OH2 | TIP | S | 59 | −17.979 | 122.996 | −70.462 | 1.00 | 66.48 | S |
| ATOM | 2378 | OH2 | TIP | S | 60 | −18.897 | 76.383 | −47.330 | 1.00 | 69.81 | S |
| ATOM | 2379 | OH2 | TIP | S | 61 | −3.494 | 95.718 | −66.750 | 1.00 | 57.69 | S |
| ATOM | 2380 | OH2 | TIP | S | 62 | −21.232 | 72.773 | −60.409 | 1.00 | 101.51 | S |
| ATOM | 2381 | OH2 | TIP | S | 63 | −24.848 | 72.699 | −43.896 | 1.00 | 69.30 | S |
| ATOM | 2382 | OH2 | TIP | S | 64 | −15.885 | 97.828 | −71.257 | 1.00 | 110.31 | S |
| ATOM | 2383 | OH2 | TIP | S | 65 | −50.982 | 93.901 | −66.391 | 1.00 | 77.23 | S |
| ATOM | 2384 | OH2 | TIP | S | 66 | −1.936 | 111.255 | −73.689 | 1.00 | 117.10 | S |
| ATOM | 2385 | OH2 | TIP | S | 67 | −36.839 | 95.982 | −64.530 | 1.00 | 66.26 | S |
| ATOM | 2386 | OH2 | TIP | S | 68 | −22.433 | 97.386 | −85.175 | 1.00 | 60.50 | S |
| ATOM | 2387 | OH2 | TIP | S | 69 | −13.273 | 99.571 | −64.786 | 1.00 | 80.33 | S |
| ATOM | 2388 | OH2 | TIP | S | 70 | −33.962 | 108.924 | −56.388 | 1.00 | 134.83 | S |
| ATOM | 2389 | OH2 | TIP | S | 71 | −18.557 | 98.422 | −71.902 | 1.00 | 99.04 | S |
| ATOM | 2390 | OH2 | TIP | S | 72 | −45.868 | 106.075 | −62.475 | 1.00 | 70.05 | S |
| ATOM | 2391 | OH2 | TIP | S | 73 | −51.210 | 97.539 | −68.618 | 1.00 | 66.94 | S |
| ATOM | 2392 | OH2 | TIP | S | 74 | −28.905 | 119.910 | −72.319 | 1.00 | 87.40 | S |
| ATOM | 2393 | OH2 | TIP | S | 75 | −28.722 | 122.818 | −67.249 | 1.00 | 79.82 | S |
| ATOM | 2394 | OH2 | TIP | S | 76 | 0.389 | 101.944 | −79.250 | 1.00 | 82.60 | S |
| ATOM | 2395 | OH2 | TIP | S | 77 | −46.387 | 108.440 | −61.129 | 1.00 | 68.36 | S |
| ATOM | 2396 | OH2 | TIP | S | 78 | −7.776 | 89.350 | −50.825 | 1.00 | 62.37 | S |
| ATOM | 2397 | OH2 | TIP | S | 79 | −49.244 | 72.524 | −53.967 | 1.00 | 69.82 | S |
| ATOM | 2398 | OH2 | TIP | S | 80 | −15.802 | 77.724 | −43.188 | 1.00 | 77.26 | S |
| ATOM | 2399 | OH2 | TIP | S | 81 | −20.198 | 84.415 | −71.895 | 1.00 | 74.18 | S |
| ATOM | 2400 | OH2 | TIP | S | 82 | −17.441 | 96.031 | −54.591 | 1.00 | 87.05 | S |
| ATOM | 2401 | OH2 | TIP | S | 83 | −13.713 | 79.061 | −53.525 | 1.00 | 103.17 | S |
| ATOM | 2402 | OH2 | TIP | S | 84 | −10.825 | 80.730 | −74.575 | 1.00 | 65.97 | S |
| ATOM | 2403 | OH2 | TIP | S | 85 | −10.354 | 92.500 | −58.649 | 1.00 | 62.83 | S |
| ATOM | 2404 | OH2 | TIP | S | 86 | −23.321 | 115.846 | −54.320 | 1.00 | 65.53 | S |
| ATOM | 2405 | OH2 | TIP | S | 87 | −16.844 | 106.078 | −51.616 | 1.00 | 63.10 | S |
| ATOM | 2406 | OH2 | TIP | S | 88 | −8.169 | 131.138 | −53.890 | 1.00 | 54.83 | S |
| ATOM | 2407 | OH2 | TIP | S | 89 | −21.698 | 115.219 | −88.740 | 1.00 | 71.18 | S |
| ATOM | 2408 | OH2 | TIP | S | 90 | −16.070 | 109.682 | −51.162 | 1.00 | 90.41 | S |
| ATOM | 2409 | OH2 | TIP | S | 91 | −49.578 | 96.512 | −57.361 | 1.00 | 94.26 | S |
| ATOM | 2410 | OH2 | TIP | S | 92 | −25.989 | 115.973 | −80.132 | 1.00 | 120.09 | S |
| ATOM | 2411 | OH2 | TIP | S | 93 | −25.659 | 128.045 | −67.099 | 1.00 | 62.69 | S |
| ATOM | 2412 | OH2 | TIP | S | 94 | −58.940 | 73.355 | −44.774 | 1.00 | 67.40 | S |
| ATOM | 2413 | OH2 | TIP | S | 95 | −7.341 | 139.735 | −51.730 | 1.00 | 67.88 | S |
| ATOM | 2414 | OH2 | TIP | S | 96 | −4.375 | 111.380 | −80.697 | 1.00 | 80.04 | S |
| ATOM | 2415 | OH2 | TIP | S | 97 | 1.015 | 101.593 | −74.683 | 1.00 | 95.35 | S |
| ATOM | 2416 | OH2 | TIP | S | 98 | −26.446 | 122.591 | −69.117 | 1.00 | 62.28 | S |
| ATOM | 2417 | OH2 | TIP | S | 99 | −13.214 | 95.620 | −57.763 | 1.00 | 59.86 | S |
| ATOM | 2418 | OH2 | TIP | S | 100 | −33.522 | 79.708 | −82.091 | 1.00 | 61.83 | S |
| ATOM | 2419 | OH2 | TIP | S | 101 | −28.979 | 113.146 | −60.493 | 1.00 | 64.27 | S |
| ATOM | 2420 | OH2 | TIP | S | 102 | −30.165 | 105.293 | −68.877 | 1.00 | 129.21 | S |
| ATOM | 2421 | OH2 | TIP | S | 103 | −20.187 | 108.049 | −60.809 | 1.00 | 95.32 | S |
| ATOM | 2422 | OH2 | TIP | S | 104 | −4.038 | 78.620 | −74.193 | 1.00 | 66.01 | S |
| ATOM | 2423 | OH2 | TIP | S | 105 | −44.998 | 72.537 | −48.705 | 1.00 | 66.95 | S |
| ATOM | 2424 | OH2 | TIP | S | 106 | −17.364 | 90.923 | −76.639 | 1.00 | 85.80 | S |
| ATOM | 2425 | OH2 | TIP | S | 107 | −24.311 | 109.750 | −67.949 | 1.00 | 125.44 | S |
| ATOM | 2426 | OH2 | TIP | S | 108 | −4.458 | 112.991 | −71.248 | 1.00 | 80.75 | S |
| ATOM | 2427 | OH2 | TIP | S | 109 | −19.788 | 112.078 | −52.627 | 1.00 | 73.74 | S |
| ATOM | 2428 | OH2 | TIP | S | 110 | −25.141 | 86.545 | −46.263 | 1.00 | 73.13 | S |
| ATOM | 2429 | OH2 | TIP | S | 111 | −9.626 | 114.187 | −70.875 | 1.00 | 47.58 | S |
| ATOM | 2430 | OH2 | TIP | S | 112 | −33.019 | 104.690 | −82.096 | 1.00 | 65.61 | S |
| ATOM | 2431 | OH2 | TIP | S | 113 | −30.915 | 103.388 | −49.239 | 1.00 | 74.91 | S |
| ATOM | 2432 | OH2 | TIP | S | 114 | −2.840 | 112.339 | −68.829 | 1.00 | 106.76 | S |
| ATOM | 2433 | OH2 | TIP | S | 115 | −5.612 | 114.639 | −65.699 | 1.00 | 93.56 | S |
| ATOM | 2434 | OH2 | TIP | S | 116 | −27.879 | 114.149 | −71.735 | 1.00 | 131.93 | S |
| ATOM | 2435 | OH2 | TIP | S | 117 | −19.800 | 76.358 | −64.870 | 1.00 | 86.29 | S |
| ATOM | 2436 | OH2 | TIP | S | 118 | −6.170 | 112.309 | −79.263 | 1.00 | 127.11 | S |
| ATOM | 2437 | OH2 | TIP | S | 119 | −17.424 | 93.166 | −52.153 | 1.00 | 116.00 | S |
| ATOM | 2438 | OH2 | TIP | S | 120 | −6.256 | 114.887 | −70.663 | 1.00 | 94.77 | S |
| ATOM | 2439 | OH2 | TIP | S | 121 | −5.429 | 109.637 | −82.500 | 1.00 | 107.88 | S |
| ATOM | 2440 | OH2 | TIP | S | 122 | −9.489 | 110.380 | −84.271 | 1.00 | 96.46 | S |
| ATOM | 2441 | OH2 | TIP | S | 123 | −34.145 | 116.425 | −70.273 | 1.00 | 113.00 | S |
| ATOM | 2442 | OH2 | TIP | S | 124 | −8.365 | 114.107 | −78.064 | 1.00 | 92.92 | S |
| ATOM | 2443 | OH2 | TIP | S | 125 | −20.890 | 121.095 | −65.288 | 1.00 | 97.32 | S |
| ATOM | 2444 | OH2 | TIP | S | 126 | −26.405 | 122.091 | −66.588 | 1.00 | 118.46 | S |
| ATOM | 2445 | OH2 | TIP | S | 127 | −38.874 | 104.293 | −58.332 | 1.00 | 97.42 | S |
| ATOM | 2446 | OH2 | TIP | S | 128 | −14.102 | 135.951 | −44.531 | 1.00 | 71.88 | S |
| ATOM | 2447 | OH2 | TIP | S | 129 | −21.085 | 97.000 | −78.847 | 1.00 | 87.18 | S |
| ATOM | 2448 | OH2 | TIP | S | 130 | −17.557 | 111.295 | −88.049 | 1.00 | 92.93 | S |
| ATOM | 2449 | OH2 | TIP | S | 131 | −20.722 | 89.291 | −49.320 | 1.00 | 68.70 | S |
| ATOM | 2450 | OH2 | TIP | S | 132 | −35.203 | 83.232 | −65.991 | 1.00 | 127.96 | S |
| ATOM | 2451 | OH2 | TIP | S | 133 | 1.796 | 94.625 | −79.513 | 1.00 | 110.26 | S |
| ATOM | 2452 | OH2 | TIP | S | 134 | −32.464 | 106.775 | −81.054 | 1.00 | 134.58 | S |
| ATOM | 2453 | OH2 | TIP | S | 135 | −17.472 | 75.316 | −55.073 | 1.00 | 67.49 | S |
| ATOM | 2454 | OH2 | TIP | S | 136 | −45.283 | 112.927 | −67.518 | 1.00 | 87.86 | S |
| ATOM | 2455 | OH2 | TIP | S | 137 | −35.109 | 94.164 | −63.986 | 1.00 | 86.94 | S |
| ATOM | 2456 | OH2 | TIP | S | 138 | −16.086 | 89.526 | −50.510 | 1.00 | 70.01 | S |
| ATOM | 2457 | OH2 | TIP | S | 139 | −23.362 | 108.488 | −64.225 | 1.00 | 154.27 | S |
| ATOM | 2458 | OH2 | TIP | S | 140 | −43.122 | 87.160 | −66.507 | 1.00 | 98.37 | S |
| ATOM | 2459 | OH2 | TIP | S | 141 | −2.776 | 109.674 | −79.736 | 1.00 | 95.96 | S |
| ATOM | 2460 | OH2 | TIP | S | 142 | −15.663 | 95.312 | −52.990 | 1.00 | 83.86 | S |
| ATOM | 2461 | OH2 | TIP | S | 143 | −14.744 | 122.625 | −70.042 | 1.00 | 100.44 | S |
| ATOM | 2462 | OH2 | TIP | S | 144 | −28.506 | 117.021 | −70.975 | 1.00 | 124.41 | S |
| ATOM | 2463 | OH2 | TIP | S | 145 | −27.738 | 90.826 | −49.482 | 1.00 | 110.44 | S |
| ATOM | 2464 | OH2 | TIP | S | 146 | −28.136 | 120.622 | −65.210 | 1.00 | 96.26 | S |
| ATOM | 2465 | OH2 | TIP | S | 147 | −20.842 | 71.154 | −62.384 | 1.00 | 111.91 | S |
| ATOM | 2466 | OH2 | TIP | S | 148 | −20.450 | 74.731 | −48.346 | 1.00 | 147.09 | S |
| ATOM | 2467 | OH2 | TIP | S | 149 | −49.750 | 90.607 | −50.890 | 1.00 | 70.14 | S |
| ATOM | 2468 | OH2 | TIP | S | 150 | −25.722 | 76.682 | −66.927 | 1.00 | 83.48 | S |
| ATOM | 2469 | OH2 | TIP | S | 151 | −10.014 | 90.765 | −70.005 | 1.00 | 67.99 | S |
| ATOM | 2470 | OH2 | TIP | S | 152 | −23.364 | 92.000 | −46.848 | 1.00 | 116.71 | S |
| ATOM | 2471 | OH2 | TIP | S | 153 | −49.912 | 95.518 | −54.399 | 1.00 | 80.82 | S |
| ATOM | 2472 | OH2 | TIP | S | 154 | −8.294 | 109.516 | −68.131 | 1.00 | 130.96 | S |
| ATOM | 2473 | OH2 | TIP | S | 155 | −35.512 | 119.480 | −62.035 | 1.00 | 92.04 | S |
| ATOM | 2474 | OH2 | TIP | S | 156 | −7.634 | 97.609 | −77.316 | 1.00 | 70.83 | S |
| ATOM | 2475 | OH2 | TIP | S | 157 | −17.429 | 105.471 | −57.223 | 1.00 | 106.41 | S |
| ATOM | 2476 | OH2 | TIP | S | 158 | −16.526 | 98.965 | −54.259 | 1.00 | 76.99 | S |
| ATOM | 2477 | OH2 | TIP | S | 159 | −11.631 | 105.626 | −81.492 | 1.00 | 72.26 | S |
| ATOM | 2478 | OH2 | TIP | S | 160 | −59.947 | 80.639 | −42.270 | 1.00 | 76.82 | S |
| ATOM | 2479 | OH2 | TIP | S | 161 | −26.927 | 80.699 | −78.815 | 1.00 | 68.78 | S |
| ATOM | 2480 | OH2 | TIP | S | 162 | −4.676 | 96.323 | −52.755 | 1.00 | 73.60 | S |
| ATOM | 2481 | OH2 | TIP | S | 163 | −27.740 | 95.995 | −59.978 | 1.00 | 102.27 | S |
| ATOM | 2482 | OH2 | TIP | S | 164 | −8.963 | 82.489 | −73.329 | 1.00 | 78.96 | S |
| ATOM | 2483 | OH2 | TIP | S | 165 | −39.727 | 100.726 | −66.210 | 1.00 | 67.68 | S |
| ATOM | 2484 | OH2 | TIP | S | 166 | −11.859 | 106.429 | −87.378 | 1.00 | 84.13 | S |
| ATOM | 2485 | OH2 | TIP | S | 167 | −18.852 | 85.938 | −39.462 | 1.00 | 70.52 | S |
| ATOM | 2486 | OH2 | TIP | S | 168 | −0.694 | 110.380 | −68.264 | 1.00 | 69.04 | S |
| ATOM | 2487 | OH2 | TIP | S | 169 | −38.128 | 78.343 | −75.080 | 1.00 | 65.00 | S |
| ATOM | 2488 | OH2 | TIP | S | 170 | −46.764 | 91.241 | −66.819 | 1.00 | 74.30 | S |
| ATOM | 2489 | OH2 | TIP | S | 171 | 3.183 | 99.399 | −79.141 | 1.00 | 128.67 | S |
| ATOM | 2490 | OH2 | TIP | S | 172 | −63.975 | 70.259 | −33.320 | 1.00 | 65.69 | S |
| ATOM | 2491 | OH2 | TIP | S | 173 | −11.493 | 123.888 | −52.294 | 1.00 | 79.10 | S |
| ATOM | 2492 | OH2 | TIP | S | 174 | −27.059 | 111.341 | −70.316 | 1.00 | 150.68 | S |
| ATOM | 2493 | OH2 | TIP | S | 175 | −15.936 | 88.989 | −62.024 | 1.00 | 97.30 | S |
| ATOM | 2494 | OH2 | TIP | S | 176 | −14.875 | 100.243 | −85.307 | 1.00 | 87.07 | S |
| ATOM | 2495 | OH2 | TIP | S | 177 | −18.037 | 70.966 | −56.508 | 1.00 | 77.36 | S |
| ATOM | 2496 | OH2 | TIP | S | 178 | −6.470 | 80.004 | −76.016 | 1.00 | 71.26 | S |
| ATOM | 2497 | OH2 | TIP | S | 179 | −49.811 | 109.689 | −67.021 | 1.00 | 63.51 | S |
| ATOM | 2498 | OH2 | TIP | S | 180 | −40.676 | 92.496 | −84.033 | 1.00 | 64.95 | S |
| ATOM | 2499 | OH2 | TIP | S | 181 | −18.587 | 98.632 | −52.482 | 1.00 | 90.86 | S |
| ATOM | 2500 | OH2 | TIP | S | 182 | −17.583 | 119.509 | −49.763 | 1.00 | 70.51 | S |
| ATOM | 2501 | OH2 | TIP | S | 183 | −11.655 | 73.314 | −55.139 | 1.00 | 75.57 | S |
| ATOM | 2502 | OH2 | TIP | S | 184 | −9.477 | 87.156 | −65.728 | 1.00 | 60.46 | S |
| ATOM | 2503 | OH2 | TIP | S | 185 | −13.688 | 110.861 | −90.110 | 1.00 | 94.57 | S |
| ATOM | 2504 | OH2 | TIP | S | 186 | −23.785 | 112.222 | −71.430 | 1.00 | 136.94 | S |
| ATOM | 2505 | OH2 | TIP | S | 187 | −39.708 | 96.995 | −50.710 | 1.00 | 81.92 | S |
| ATOM | 2506 | OH2 | TIP | S | 188 | −36.265 | 93.471 | −86.062 | 1.00 | 69.78 | S |
| ATOM | 2507 | OH2 | TIP | S | 189 | −13.807 | 119.103 | −74.658 | 1.00 | 124.21 | S |
| ATOM | 2508 | OH2 | TIP | S | 190 | 2.032 | 95.724 | −83.054 | 1.00 | 74.44 | S |
| ATOM | 2509 | OH2 | TIP | S | 191 | −33.006 | 112.815 | −70.272 | 1.00 | 113.66 | S |
| ATOM | 2510 | OH2 | TIP | S | 192 | −40.901 | 76.884 | −74.050 | 1.00 | 86.83 | S |
| ATOM | 2511 | OH2 | TIP | S | 193 | −46.882 | 97.000 | −55.824 | 1.00 | 65.96 | S |
| ATOM | 2512 | OH2 | TIP | S | 194 | −15.147 | 95.684 | −76.401 | 1.00 | 95.85 | S |
| ATOM | 2513 | OH2 | TIP | S | 195 | −13.498 | 80.649 | −60.434 | 1.00 | 102.97 | S |
| ATOM | 2514 | OH2 | TIP | S | 196 | −22.522 | 78.860 | −64.110 | 1.00 | 121.19 | S |
| ATOM | 2515 | OH2 | TIP | S | 197 | −29.314 | 84.793 | −82.436 | 1.00 | 84.45 | S |
| END | | | | | | | | | | | |

TABLE 4
Coordinate data for ΔPH-PKBβ-ΔC

CRYST1 149.703 149.703 39.185 90.00 90.00 90.00 P 41 21 2
REMARK FILENAME = “dph-pkb-dc.pdb”
REMARK DATE: 30-Nov-01 15:41:04
REMARK VERSION: 1.1

| | | | | | | | | | | | |
|---|---|---|---|---|---|---|---|---|---|---|---|
| ATOM | 1 | CB | ALA | | 146 | −38.368 | 81.177 | −47.423 | 1.00 | 72.10 | |
| ATOM | 2 | C | ALA | | 146 | −38.291 | 81.513 | −49.914 | 1.00 | 73.26 | |
| ATOM | 3 | O | ALA | | 146 | −38.678 | 80.383 | −50.231 | 1.00 | 74.20 | |
| ATOM | 4 | N | ALA | | 146 | −40.134 | 82.542 | −48.532 | 1.00 | 72.62 | |
| ATOM | 5 | CA | ALA | | 146 | −38.688 | 82.145 | −48.572 | 1.00 | 73.27 | |
| ATOM | 6 | N | ALA | | 147 | −37.512 | 82.257 | −50.696 | 1.00 | 72.37 | |
| ATOM | 7 | CA | ALA | | 147 | −37.051 | 81.793 | −51.995 | 1.00 | 70.36 | |
| ATOM | 8 | CB | ALA | | 147 | −36.169 | 82.851 | −52.631 | 1.00 | 70.01 | |
| ATOM | 9 | C | ALA | | 147 | −36.275 | 80.488 | −51.849 | 1.00 | 69.01 | |
| ATOM | 10 | O | ALA | | 147 | −35.807 | 80.142 | −50.764 | 1.00 | 68.52 | |
| ATOM | 11 | N | THR | | 148 | −36.147 | 79.771 | −52.957 | 1.00 | 67.57 | |
| ATOM | 12 | CA | THR | | 148 | −35.416 | 78.514 | −52.995 | 1.00 | 66.30 | |
| ATOM | 13 | CB | THR | | 148 | −36.130 | 77.423 | −52.159 | 1.00 | 66.09 | |
| ATOM | 14 | OG1 | THR | | 148 | −35.396 | 77.229 | −50.947 | 1.00 | 67.43 | |
| ATOM | 15 | CG2 | THR | | 148 | −36.220 | 76.101 | −52.907 | 1.00 | 64.34 | |
| ATOM | 16 | C | THR | | 148 | −35.268 | 78.077 | −54.443 | 1.00 | 66.26 | |
| ATOM | 17 | O | THR | | 148 | −36.141 | 78.339 | −55.271 | 1.00 | 65.96 | |
| ATOM | 18 | N | MET | | 149 | −34.154 | 77.424 | −54.746 | 1.00 | 65.69 | |
| ATOM | 19 | CA | MET | | 149 | −33.882 | 76.969 | −56.099 | 1.00 | 65.90 | |
| ATOM | 20 | CB | MET | | 149 | −32.779 | 75.923 | −56.075 | 1.00 | 65.89 | |
| ATOM | 21 | CG | MET | | 149 | −31.452 | 76.489 | −55.657 | 1.00 | 66.44 | |
| ATOM | 22 | SD | MET | | 149 | −30.870 | 77.680 | −56.856 | 1.00 | 65.10 | |
| ATOM | 23 | CE | MET | | 149 | −29.158 | 77.222 | −56.912 | 1.00 | 67.52 | |
| ATOM | 24 | C | MET | | 149 | −35.098 | 76.405 | −56.805 | 1.00 | 66.12 | |
| ATOM | 25 | O | MET | | 149 | −35.324 | 76.688 | −57.980 | 1.00 | 66.35 | |
| ATOM | 26 | N | ASN | | 150 | −35.886 | 75.619 | −56.077 | 1.00 | 66.02 | |
| ATOM | 27 | CA | ASN | | 150 | −37.073 | 74.989 | −56.641 | 1.00 | 65.30 | |
| ATOM | 28 | CB | ASN | | 150 | −37.574 | 73.882 | −55.718 | 1.00 | 64.72 | |
| ATOM | 29 | CG | ASN | | 150 | −38.585 | 72.996 | −56.399 | 1.00 | 66.37 | |
| ATOM | 30 | OD1 | ASN | | 150 | −39.424 | 72.371 | −55.753 | 1.00 | 68.35 | |
| ATOM | 31 | ND2 | ASN | | 150 | −38.510 | 72.932 | −57.725 | 1.00 | 66.81 | |
| ATOM | 32 | C | ASN | | 150 | −38.231 | 75.947 | −56.964 | 1.00 | 64.97 | |
| ATOM | 33 | O | ASN | | 150 | −39.215 | 75.552 | −57.591 | 1.00 | 64.68 | |
| ATOM | 34 | N | ASP | | 151 | −38.125 | 77.199 | −56.539 | 1.00 | 64.20 | |
| ATOM | 35 | CA | ASP | | 151 | −39.173 | 78.152 | −56.831 | 1.00 | 64.01 | |
| ATOM | 36 | CB | ASP | | 151 | −39.178 | 79.279 | −55.800 | 1.00 | 65.85 | |
| ATOM | 37 | CG | ASP | | 151 | −39.625 | 78.816 | −54.426 | 1.00 | 67.83 | |
| ATOM | 38 | OD1 | ASP | | 151 | −40.580 | 78.011 | −54.361 | 1.00 | 69.12 | |
| ATOM | 39 | OD2 | ASP | | 151 | −39.041 | 79.268 | −53.413 | 1.00 | 68.47 | |
| ATOM | 40 | C | ASP | | 151 | −38.979 | 78.746 | −58.221 | 1.00 | 63.35 | |
| ATOM | 41 | O | ASP | | 151 | −39.803 | 79.536 | −58.679 | 1.00 | 63.55 | |
| ATOM | 42 | N | PHE | | 152 | −37.901 | 78.359 | −58.902 | 1.00 | 62.43 | |
| ATOM | 43 | CA | PHE | | 152 | −37.608 | 78.904 | −60.233 | 1.00 | 61.46 | |
| ATOM | 44 | CB | PHE | | 152 | −36.387 | 79.842 | −60.174 | 1.00 | 60.80 | |
| ATOM | 45 | CG | PHE | | 152 | −36.484 | 80.896 | −59.123 | 1.00 | 59.27 | |
| ATOM | 46 | CD1 | PHE | | 152 | −37.244 | 82.029 | −59.333 | 1.00 | 58.29 | |
| ATOM | 47 | CD2 | PHE | | 152 | −35.878 | 80.714 | −57.887 | 1.00 | 59.08 | |
| ATOM | 48 | CE1 | PHE | | 152 | −37.411 | 82.968 | −58.324 | 1.00 | 58.92 | |
| ATOM | 49 | CE2 | PHE | | 152 | −36.041 | 81.641 | −56.877 | 1.00 | 58.79 | |
| ATOM | 50 | CZ | PHE | | 152 | −36.813 | 82.773 | −57.095 | 1.00 | 58.93 | |
| ATOM | 51 | C | PHE | | 152 | −37.331 | 77.865 | −61.313 | 1.00 | 61.40 | |
| ATOM | 52 | O | PHE | | 152 | −36.913 | 76.734 | −61.028 | 1.00 | 61.27 | |
| ATOM | 53 | N | ASP | | 153 | −37.551 | 78.281 | −62.560 | 1.00 | 59.89 | |
| ATOM | 54 | CA | ASP | | 153 | −37.298 | 77.449 | −63.723 | 1.00 | 58.75 | |
| ATOM | 55 | CB | ASP | | 153 | −38.509 | 77.446 | −64.667 | 1.00 | 62.22 | |
| ATOM | 56 | CG | ASP | | 153 | −39.670 | 76.575 | −64.156 | 1.00 | 63.93 | |
| ATOM | 57 | OD1 | ASP | | 153 | −39.507 | 75.338 | −64.029 | 1.00 | 63.98 | |
| ATOM | 58 | OD2 | ASP | | 153 | −40.755 | 77.136 | −63.888 | 1.00 | 64.53 | |
| ATOM | 59 | C | ASP | | 153 | −36.089 | 78.019 | −64.461 | 1.00 | 57.30 | |
| ATOM | 60 | O | ASP | | 153 | −35.975 | 79.234 | −64.644 | 1.00 | 57.33 | |
| ATOM | 61 | N | TYR | | 154 | −35.190 | 77.136 | −64.881 | 1.00 | 55.23 | |
| ATOM | 62 | CA | TYR | | 154 | −33.995 | 77.530 | −65.611 | 1.00 | 53.89 | |
| ATOM | 63 | CB | TYR | | 154 | −32.969 | 76.411 | −65.565 | 1.00 | 51.68 | |
| ATOM | 64 | CG | TYR | | 154 | −31.788 | 76.648 | −66.476 | 1.00 | 52.25 | |
| ATOM | 65 | CD1 | TYR | | 154 | −30.763 | 77.530 | −66.124 | 1.00 | 53.16 | |
| ATOM | 66 | CE1 | TYR | | 154 | −29.662 | 77.724 | −66.960 | 1.00 | 52.88 | |
| ATOM | 67 | CD2 | TYR | | 154 | −31.681 | 75.976 | −67.679 | 1.00 | 51.34 | |
| ATOM | 68 | CE2 | TYR | | 154 | −30.593 | 76.154 | −68.511 | 1.00 | 52.96 | |
| ATOM | 69 | CZ | TYR | | 154 | −29.583 | 77.022 | −68.154 | 1.00 | 53.56 | |
| ATOM | 70 | OH | TYR | | 154 | −28.485 | 77.141 | −68.987 | 1.00 | 54.21 | |
| ATOM | 71 | C | TYR | | 154 | −34.306 | 77.835 | −67.071 | 1.00 | 54.58 | |
| ATOM | 72 | O | TYR | | 154 | −35.036 | 77.099 | −67.710 | 1.00 | 58.05 | |
| ATOM | 73 | N | LEU | | 155 | −33.758 | 78.913 | −67.611 | 1.00 | 54.64 | |
| ATOM | 74 | CA | LEU | | 155 | −33.990 | 79.234 | −69.011 | 1.00 | 53.24 | |
| ATOM | 75 | CB | LEU | | 155 | −34.665 | 80.590 | −69.164 | 1.00 | 52.10 | |
| ATOM | 76 | CG | LEU | | 155 | −36.138 | 80.449 | −68.807 | 1.00 | 52.18 | |
| ATOM | 77 | CD1 | LEU | | 155 | −36.824 | 81.788 | −68.842 | 1.00 | 53.57 | |
| ATOM | 78 | CD2 | LEU | | 155 | −36.782 | 79.508 | −69.785 | 1.00 | 53.33 | |
| ATOM | 79 | C | LEU | | 155 | −32.681 | 79.226 | −69.744 | 1.00 | 53.55 | |
| ATOM | 80 | O | LEU | | 155 | −32.469 | 78.417 | −70.643 | 1.00 | 53.03 | |
| ATOM | 81 | N | LYS | | 156 | −31.781 | 80.118 | −69.371 | 1.00 | 53.89 | |
| ATOM | 82 | CA | LYS | | 156 | −30.513 | 80.114 | −70.058 | 1.00 | 55.50 | |
| ATOM | 83 | CB | LYS | | 156 | −30.667 | 80.746 | −71.443 | 1.00 | 56.37 | |
| ATOM | 84 | CG | LYS | | 156 | −31.083 | 82.208 | −71.429 | 1.00 | 57.54 | |
| ATOM | 85 | CD | LYS | | 156 | −30.759 | 82.836 | −72.769 | 1.00 | 58.31 | |
| ATOM | 86 | CE | LYS | | 156 | −30.520 | 84.321 | −72.634 | 1.00 | 57.68 | |
| ATOM | 87 | NZ | LYS | | 156 | −29.917 | 84.844 | −73.888 | 1.00 | 58.86 | |
| ATOM | 88 | C | LYS | | 156 | −29.402 | 80.799 | −69.297 | 1.00 | 56.19 | |
| ATOM | 89 | O | LYS | | 156 | −29.629 | 81.458 | −68.278 | 1.00 | 55.60 | |
| ATOM | 90 | N | LEU | | 157 | −28.188 | 80.623 | −69.795 | 1.00 | 57.43 | |
| ATOM | 91 | CA | LEU | | 157 | −27.050 | 81.243 | −69.168 | 1.00 | 59.59 | |
| ATOM | 92 | CB | LEU | | 157 | −25.772 | 80.504 | −69.533 | 1.00 | 58.98 | |
| ATOM | 93 | CG | LEU | | 157 | −24.501 | 81.061 | −68.892 | 1.00 | 58.30 | |
| ATOM | 94 | CD1 | LEU | | 157 | −24.764 | 81.446 | −67.457 | 1.00 | 58.21 | |
| ATOM | 95 | CD2 | LEU | | 157 | −23.406 | 80.017 | −68.963 | 1.00 | 58.74 | |
| ATOM | 96 | C | LEU | | 157 | −26.969 | 82.672 | −69.666 | 1.00 | 61.07 | |
| ATOM | 97 | O | LEU | | 157 | −27.146 | 82.927 | −70.857 | 1.00 | 61.45 | |
| ATOM | 98 | N | LEU | | 158 | −26.729 | 83.597 | −68.743 | 1.00 | 61.77 | |
| ATOM | 99 | CA | LEU | | 158 | −26.603 | 85.004 | −69.074 | 1.00 | 63.79 | |
| ATOM | 100 | CB | LEU | | 158 | −27.477 | 85.862 | −68.161 | 1.00 | 63.54 | |
| ATOM | 101 | CG | LEU | | 158 | −28.985 | 85.959 | −68.361 | 1.00 | 62.03 | |
| ATOM | 102 | CD1 | LEU | | 158 | −29.582 | 86.501 | −67.080 | 1.00 | 63.45 | |
| ATOM | 103 | CD2 | LEU | | 158 | −29.331 | 86.859 | −69.536 | 1.00 | 61.08 | |
| ATOM | 104 | C | LEU | | 158 | −25.155 | 85.409 | −68.878 | 1.00 | 65.26 | |
| ATOM | 105 | O | LEU | | 158 | −24.663 | 86.298 | −69.564 | 1.00 | 64.54 | |
| ATOM | 106 | N | GLY | | 159 | −24.474 | 84.760 | −67.934 | 1.00 | 68.01 | |
| ATOM | 107 | CA | GLY | | 159 | −23.078 | 85.091 | −67.678 | 1.00 | 71.21 | |
| ATOM | 108 | C | GLY | | 159 | −22.361 | 84.198 | −66.678 | 1.00 | 73.75 | |
| ATOM | 109 | O | GLY | | 159 | −22.970 | 83.688 | −65.736 | 1.00 | 73.94 | |
| ATOM | 110 | N | LYS | | 160 | −21.058 | 84.016 | −66.883 | 1.00 | 76.25 | |
| ATOM | 111 | CA | LYS | | 160 | −20.222 | 83.178 | −66.011 | 1.00 | 78.92 | |
| ATOM | 112 | CB | LYS | | 160 | −19.635 | 82.023 | −66.851 | 1.00 | 78.80 | |
| ATOM | 113 | CG | LYS | | 160 | −19.312 | 80.735 | −66.086 | 1.00 | 79.84 | |
| ATOM | 114 | CD | LYS | | 160 | −18.660 | 79.651 | −66.992 | 1.00 | 79.69 | |
| ATOM | 115 | CE | LYS | | 160 | −18.214 | 78.394 | −66.183 | 1.00 | 79.04 | |
| ATOM | 116 | NZ | LYS | | 160 | −17.218 | 77.517 | −66.886 | 1.00 | 75.82 | |
| ATOM | 117 | C | LYS | | 160 | −19.096 | 84.059 | −65.399 | 1.00 | 80.46 | |
| ATOM | 118 | O | LYS | | 160 | −19.049 | 85.271 | −65.647 | 1.00 | 81.00 | |
| ATOM | 119 | N | GLY | | 161 | −18.199 | 83.481 | −64.597 | 1.00 | 81.61 | |
| ATOM | 120 | CA | GLY | | 161 | −17.131 | 84.301 | −64.032 | 1.00 | 82.86 | |
| ATOM | 121 | C | GLY | | 161 | −16.242 | 83.756 | −62.918 | 1.00 | 83.67 | |
| ATOM | 122 | O | GLY | | 161 | −16.419 | 82.639 | −62.426 | 1.00 | 83.60 | |
| ATOM | 123 | N | THR | | 162 | −15.273 | 84.573 | −62.519 | 1.00 | 84.29 | |
| ATOM | 124 | CA | THR | | 162 | −14.335 | 84.215 | −61.460 | 1.00 | 85.61 | |
| ATOM | 125 | CB | THR | | 162 | −13.167 | 85.245 | −61.391 | 1.00 | 87.59 | |
| ATOM | 126 | OG1 | THR | | 162 | −13.056 | 85.934 | −62.648 | 1.00 | 89.49 | |
| ATOM | 127 | CG2 | THR | | 162 | −11.837 | 84.543 | −61.087 | 1.00 | 88.57 | |
| ATOM | 128 | C | THR | | 162 | −15.080 | 84.230 | −60.122 | 1.00 | 84.92 | |
| ATOM | 129 | O | THR | | 162 | −14.579 | 83.753 | −59.105 | 1.00 | 84.61 | |
| ATOM | 130 | N | PHE | | 163 | −16.286 | 84.786 | −60.148 | 1.00 | 84.72 | |
| ATOM | 131 | CA | PHE | | 163 | −17.145 | 84.914 | −58.968 | 1.00 | 84.07 | |
| ATOM | 132 | CB | PHE | | 163 | −17.718 | 86.338 | −58.947 | 1.00 | 84.30 | |
| ATOM | 133 | CG | PHE | | 163 | −18.371 | 86.753 | −60.251 | 1.00 | 85.54 | |
| ATOM | 134 | CD1 | PHE | | 163 | −19.759 | 86.669 | −60.419 | 1.00 | 85.14 | |
| ATOM | 135 | CD2 | PHE | | 163 | −17.597 | 87.221 | −61.315 | 1.00 | 85.78 | |
| ATOM | 136 | CE1 | PHE | | 163 | −20.371 | 87.048 | −61.623 | 1.00 | 85.09 | |
| ATOM | 137 | CE2 | PHE | | 163 | −18.197 | 87.602 | −62.526 | 1.00 | 86.86 | |
| ATOM | 138 | CZ | PHE | | 163 | −19.591 | 87.516 | −62.677 | 1.00 | 86.20 | |
| ATOM | 139 | C | PHE | | 163 | −18.291 | 83.867 | −58.929 | 1.00 | 82.93 | |
| ATOM | 140 | O | PHE | | 163 | −18.651 | 83.340 | −57.864 | 1.00 | 82.41 | |
| ATOM | 141 | N | GLY | | 164 | −18.849 | 83.573 | −60.100 | 1.00 | 80.55 | |
| ATOM | 142 | CA | GLY | | 164 | −19.936 | 82.618 | −60.202 | 1.00 | 77.40 | |
| ATOM | 143 | C | GLY | | 164 | −20.599 | 82.701 | −61.569 | 1.00 | 75.05 | |
| ATOM | 144 | O | GLY | | 164 | −19.925 | 82.648 | −62.606 | 1.00 | 75.87 | |
| ATOM | 145 | N | LYS | | 165 | −21.918 | 82.836 | −61.593 | 1.00 | 72.00 | |
| ATOM | 146 | CA | LYS | | 165 | −22.593 | 82.923 | −62.871 | 1.00 | 68.85 | |
| ATOM | 147 | CB | LYS | | 165 | −22.699 | 81.524 | −63.488 | 1.00 | 70.37 | |
| ATOM | 148 | CG | LYS | | 165 | −23.766 | 80.630 | −62.864 | 1.00 | 71.00 | |
| ATOM | 149 | CD | LYS | | 165 | −23.663 | 79.198 | −63.358 | 1.00 | 72.16 | |
| ATOM | 150 | CE | LYS | | 165 | −22.585 | 78.449 | −62.588 | 1.00 | 74.70 | |
| ATOM | 151 | NZ | LYS | | 165 | −22.532 | 77.000 | −62.927 | 1.00 | 76.05 | |
| ATOM | 152 | C | LYS | | 165 | −23.974 | 83.524 | −62.712 | 1.00 | 66.56 | |
| ATOM | 153 | O | LYS | | 165 | −24.522 | 83.553 | −61.610 | 1.00 | 65.74 | |
| ATOM | 154 | N | VAL | | 166 | −24.520 | 84.027 | −63.815 | 1.00 | 63.53 | |
| ATOM | 155 | CA | VAL | | 166 | −25.862 | 84.583 | −63.803 | 1.00 | 61.12 | |
| ATOM | 156 | CB | VAL | | 166 | −25.894 | 86.080 | −64.173 | 1.00 | 62.20 | |
| ATOM | 157 | CG1 | VAL | | 166 | −27.339 | 86.566 | −64.227 | 1.00 | 61.08 | |
| ATOM | 158 | CG2 | VAL | | 166 | −25.120 | 86.891 | −63.135 | 1.00 | 62.28 | |
| ATOM | 159 | C | VAL | | 166 | −26.689 | 83.792 | −64.798 | 1.00 | 58.96 | |
| ATOM | 160 | O | VAL | | 166 | −26.345 | 83.686 | −65.972 | 1.00 | 56.35 | |
| ATOM | 161 | N | ILE | | 167 | −27.771 | 83.224 | −64.282 | 1.00 | 58.40 | |
| ATOM | 162 | CA | ILE | | 167 | −28.714 | 82.403 | −65.026 | 1.00 | 57.37 | |
| ATOM | 163 | CB | ILE | | 167 | −29.121 | 81.170 | −64.170 | 1.00 | 56.62 | |
| ATOM | 164 | CG2 | ILE | | 167 | −30.181 | 80.366 | −64.857 | 1.00 | 58.67 | |
| ATOM | 165 | CG1 | ILE | | 167 | −27.922 | 80.276 | −63.941 | 1.00 | 56.70 | |
| ATOM | 166 | CD1 | ILE | | 167 | −27.326 | 79.765 | −65.237 | 1.00 | 57.59 | |
| ATOM | 167 | C | ILE | | 167 | −29.968 | 83.229 | −65.287 | 1.00 | 56.93 | |
| ATOM | 168 | O | ILE | | 167 | −30.276 | 84.155 | −64.533 | 1.00 | 57.78 | |
| ATOM | 169 | N | LEU | | 168 | −30.682 | 82.913 | −66.359 | 1.00 | 55.71 | |
| ATOM | 170 | CA | LEU | | 168 | −31.942 | 83.588 | −66.656 | 1.00 | 55.34 | |
| ATOM | 171 | CB | LEU | | 168 | −32.086 | 83.818 | −68.159 | 1.00 | 54.97 | |
| ATOM | 172 | CG | LEU | | 168 | −33.334 | 84.504 | −68.725 | 1.00 | 55.00 | |
| ATOM | 173 | CD1 | LEU | | 168 | −33.637 | 85.838 | −68.041 | 1.00 | 54.88 | |
| ATOM | 174 | CD2 | LEU | | 168 | −33.073 | 84.724 | −70.197 | 1.00 | 55.25 | |
| ATOM | 175 | C | LEU | | 168 | −32.981 | 82.577 | −66.186 | 1.00 | 55.65 | |
| ATOM | 176 | O | LEU | | 168 | −33.072 | 81.488 | −66.750 | 1.00 | 56.14 | |
| ATOM | 177 | N | VAL | | 169 | −33.743 | 82.914 | −65.149 | 1.00 | 56.28 | |
| ATOM | 178 | CA | VAL | | 169 | −34.744 | 81.991 | −64.606 | 1.00 | 56.68 | |
| ATOM | 179 | CB | VAL | | 169 | −34.394 | 81.567 | −63.153 | 1.00 | 55.72 | |
| ATOM | 180 | CG1 | VAL | | 169 | −33.016 | 80.927 | −63.105 | 1.00 | 53.79 | |
| ATOM | 181 | CG2 | VAL | | 169 | −34.444 | 82.776 | −62.231 | 1.00 | 52.73 | |
| ATOM | 182 | C | VAL | | 169 | −36.133 | 82.605 | −64.575 | 1.00 | 58.31 | |
| ATOM | 183 | O | VAL | | 169 | −36.277 | 83.810 | −64.750 | 1.00 | 57.96 | |
| ATOM | 184 | N | ARG | | 170 | −37.145 | 81.771 | −64.335 | 1.00 | 60.42 | |
| ATOM | 185 | CA | ARG | | 170 | −38.535 | 82.222 | −64.256 | 1.00 | 62.62 | |
| ATOM | 186 | CB | ARG | | 170 | −39.344 | 81.620 | −65.405 | 1.00 | 64.57 | |
| ATOM | 187 | CG | ARG | | 170 | −40.685 | 82.328 | −65.671 | 1.00 | 68.47 | |
| ATOM | 188 | CD | ARG | | 170 | −41.696 | 81.470 | −66.464 | 1.00 | 69.77 | |
| ATOM | 189 | NE | ARG | | 170 | −41.088 | 80.712 | −67.562 | 1.00 | 71.23 | |
| ATOM | 190 | CZ | ARG | | 170 | −40.826 | 79.406 | −67.514 | 1.00 | 71.25 | |
| ATOM | 191 | NH1 | ARG | | 170 | −41.122 | 78.706 | −66.424 | 1.00 | 70.34 | |
| ATOM | 192 | NH2 | ARG | | 170 | −40.259 | 78.802 | −68.553 | 1.00 | 70.83 | |
| ATOM | 193 | C | ARG | | 170 | −39.198 | 81.813 | −62.930 | 1.00 | 63.43 | |
| ATOM | 194 | O | ARG | | 170 | −39.399 | 80.632 | −62.688 | 1.00 | 64.23 | |
| ATOM | 195 | N | GLU | | 171 | −39.541 | 82.774 | −62.074 | 1.00 | 64.60 | |
| ATOM | 196 | CA | GLU | | 171 | −40.199 | 82.444 | −60.809 | 1.00 | 66.03 | |
| ATOM | 197 | CB | GLU | | 171 | −40.527 | 83.720 | −60.014 | 1.00 | 66.33 | |
| ATOM | 198 | CG | GLU | | 171 | −41.455 | 83.497 | −58.804 | 1.00 | 66.72 | |
| ATOM | 199 | CD | GLU | | 171 | −41.511 | 84.693 | −57.842 | 1.00 | 67.61 | |
| ATOM | 200 | OE1 | GLU | | 171 | −41.639 | 85.855 | −58.311 | 1.00 | 66.75 | |
| ATOM | 201 | OE2 | GLU | | 171 | −41.442 | 84.457 | −56.612 | 1.00 | 65.68 | |
| ATOM | 202 | C | GLU | | 171 | −41.481 | 81.669 | −61.118 | 1.00 | 67.13 | |
| ATOM | 203 | O | GLU | | 171 | −42.431 | 82.226 | −61.680 | 1.00 | 67.09 | |
| ATOM | 204 | N | LYS | | 172 | −41.510 | 80.391 | −60.734 | 1.00 | 67.98 | |
| ATOM | 205 | CA | LYS | | 172 | −42.655 | 79.523 | −61.011 | 1.00 | 68.77 | |
| ATOM | 206 | CB | LYS | | 172 | −42.374 | 78.107 | −60.511 | 1.00 | 67.84 | |
| ATOM | 207 | CG | LYS | | 172 | −41.981 | 77.172 | −61.634 | 1.00 | 68.40 | |
| ATOM | 208 | CD | LYS | | 172 | −41.645 | 75.766 | −61.157 | 1.00 | 68.66 | |
| ATOM | 209 | CE | LYS | | 172 | −40.306 | 75.704 | −60.454 | 1.00 | 68.13 | |
| ATOM | 210 | NZ | LYS | | 172 | −40.126 | 74.417 | −59.745 | 1.00 | 66.77 | |
| ATOM | 211 | C | LYS | | 172 | −44.046 | 79.951 | −60.548 | 1.00 | 69.73 | |
| ATOM | 212 | O | LYS | | 172 | −45.037 | 79.630 | −61.199 | 1.00 | 69.61 | |
| ATOM | 213 | N | ALA | | 173 | −44.139 | 80.663 | −59.433 | 1.00 | 71.28 | |
| ATOM | 214 | CA | ALA | | 173 | −45.449 | 81.089 | −58.949 | 1.00 | 72.19 | |
| ATOM | 215 | CB | ALA | | 173 | −45.330 | 81.689 | −57.545 | 1.00 | 72.40 | |
| ATOM | 216 | C | ALA | | 173 | −46.046 | 82.115 | −59.899 | 1.00 | 72.23 | |
| ATOM | 217 | O | ALA | | 173 | −47.053 | 81.856 | −60.564 | 1.00 | 71.61 | |
| ATOM | 218 | N | THR | | 174 | −45.400 | 83.276 | −59.960 | 1.00 | 71.76 | |
| ATOM | 219 | CA | THR | | 174 | −45.858 | 84.365 | −60.799 | 1.00 | 71.25 | |
| ATOM | 220 | CB | THR | | 174 | −45.088 | 85.660 | −60.488 | 1.00 | 71.18 | |
| ATOM | 221 | OG1 | THR | | 174 | −43.845 | 85.668 | −61.195 | 1.00 | 70.70 | |
| ATOM | 222 | CG2 | THR | | 174 | −44.804 | 85.756 | −58.998 | 1.00 | 71.39 | |
| ATOM | 223 | C | THR | | 174 | −45.712 | 84.044 | −62.278 | 1.00 | 71.45 | |
| ATOM | 224 | O | THR | | 174 | −46.703 | 83.867 | −62.989 | 1.00 | 73.06 | |
| ATOM | 225 | N | GLY | | 175 | −44.475 | 83.955 | −62.737 | 1.00 | 71.40 | |
| ATOM | 226 | CA | GLY | | 175 | −44.224 | 83.682 | −64.134 | 1.00 | 71.12 | |
| ATOM | 227 | C | GLY | | 175 | −43.263 | 84.738 | −64.624 | 1.00 | 71.06 | |
| ATOM | 228 | O | GLY | | 175 | −42.784 | 84.680 | −65.757 | 1.00 | 71.69 | |
| ATOM | 229 | N | ARG | | 176 | −42.979 | 85.703 | −63.752 | 1.00 | 70.93 | |
| ATOM | 230 | CA | ARG | | 176 | −42.067 | 86.795 | −64.063 | 1.00 | 72.13 | |
| ATOM | 231 | CB | ARG | | 176 | −42.083 | 87.836 | −62.930 | 1.00 | 75.41 | |
| ATOM | 232 | CG | ARG | | 176 | −43.441 | 88.495 | −62.650 | 1.00 | 79.99 | |
| ATOM | 233 | CD | ARG | | 176 | −43.962 | 89.314 | −63.842 | 1.00 | 84.22 | |
| ATOM | 234 | NE | ARG | | 176 | −45.334 | 89.793 | −63.623 | 1.00 | 87.99 | |
| ATOM | 235 | CZ | ARG | | 176 | −46.092 | 90.395 | −64.543 | 1.00 | 88.86 | |
| ATOM | 236 | NH1 | ARG | | 176 | −45.625 | 90.607 | −65.772 | 1.00 | 89.64 | |
| ATOM | 237 | NH2 | ARG | | 176 | −47.327 | 90.783 | −64.233 | 1.00 | 88.39 | |
| ATOM | 238 | C | ARG | | 176 | −40.636 | 86.271 | −64.272 | 1.00 | 71.38 | |
| ATOM | 239 | O | ARG | | 176 | −40.294 | 85.178 | −63.818 | 1.00 | 71.09 | |
| ATOM | 240 | N | TYR | | 177 | −39.805 | 87.067 | −64.948 | 1.00 | 69.68 | |
| ATOM | 241 | CA | TYR | | 177 | −38.419 | 86.701 | −65.242 | 1.00 | 67.28 | |
| ATOM | 242 | CB | TYR | | 177 | −38.129 | 86.903 | −66.727 | 1.00 | 67.29 | |
| ATOM | 243 | CG | TYR | | 177 | −38.865 | 85.978 | −67.648 | 1.00 | 68.57 | |
| ATOM | 244 | CD1 | TYR | | 177 | −40.249 | 85.907 | −67.637 | 1.00 | 68.90 | |
| ATOM | 245 | CE1 | TYR | | 177 | −40.925 | 85.056 | −68.498 | 1.00 | 69.12 | |
| ATOM | 246 | CD2 | TYR | | 177 | −38.174 | 85.168 | −68.542 | 1.00 | 69.83 | |
| ATOM | 247 | CE2 | TYR | | 177 | −38.845 | 84.318 | −69.406 | 1.00 | 69.44 | |
| ATOM | 248 | CZ | TYR | | 177 | −40.214 | 84.266 | −69.374 | 1.00 | 68.87 | |
| ATOM | 249 | OH | TYR | | 177 | −40.877 | 83.426 | −70.226 | 1.00 | 70.45 | |
| ATOM | 250 | C | TYR | | 177 | −37.363 | 87.476 | −64.456 | 1.00 | 66.16 | |
| ATOM | 251 | O | TYR | | 177 | −37.376 | 88.712 | −64.406 | 1.00 | 65.73 | |
| ATOM | 252 | N | TYR | | 178 | −36.430 | 86.741 | −63.865 | 1.00 | 65.15 | |
| ATOM | 253 | CA | TYR | | 178 | −35.342 | 87.359 | −63.121 | 1.00 | 63.26 | |
| ATOM | 254 | CB | TYR | | 178 | −35.492 | 87.108 | −61.622 | 1.00 | 62.91 | |
| ATOM | 255 | CG | TYR | | 178 | −36.817 | 87.567 | −61.062 | 1.00 | 63.24 | |
| ATOM | 256 | CD1 | TYR | | 178 | −37.991 | 86.857 | −61.327 | 1.00 | 63.61 | |
| ATOM | 257 | CE1 | TYR | | 178 | −39.225 | 87.300 | −60.850 | 1.00 | 63.23 | |
| ATOM | 258 | CD2 | TYR | | 178 | −36.909 | 88.734 | −60.299 | 1.00 | 63.38 | |
| ATOM | 259 | CE2 | TYR | | 178 | −38.137 | 89.183 | −59.819 | 1.00 | 63.89 | |
| ATOM | 260 | CZ | TYR | | 178 | −39.289 | 88.459 | −60.100 | 1.00 | 63.86 | |
| ATOM | 261 | OH | TYR | | 178 | −40.506 | 88.893 | −59.632 | 1.00 | 66.00 | |
| ATOM | 262 | C | TYR | | 178 | −34.017 | 86.785 | −63.598 | 1.00 | 62.05 | |
| ATOM | 263 | O | TYR | | 178 | −33.967 | 85.775 | −64.303 | 1.00 | 61.10 | |
| ATOM | 264 | N | ALA | | 179 | −32.943 | 87.460 | −63.241 | 1.00 | 60.77 | |
| ATOM | 265 | CA | ALA | | 179 | −31.630 | 86.987 | −63.606 | 1.00 | 60.99 | |
| ATOM | 266 | CB | ALA | | 179 | −30.807 | 88.114 | −64.165 | 1.00 | 62.12 | |
| ATOM | 267 | C | ALA | | 179 | −31.083 | 86.550 | −62.274 | 1.00 | 60.52 | |
| ATOM | 268 | O | ALA | | 179 | −31.094 | 87.339 | −61.327 | 1.00 | 60.98 | |
| ATOM | 269 | N | MET | | 180 | −30.627 | 85.304 | −62.179 | 1.00 | 59.47 | |
| ATOM | 270 | CA | MET | | 180 | −30.104 | 84.818 | −60.909 | 1.00 | 57.96 | |
| ATOM | 271 | CB | MET | | 180 | −30.636 | 83.436 | −60.591 | 1.00 | 55.07 | |
| ATOM | 272 | CG | MET | | 180 | −30.452 | 83.096 | −59.142 | 1.00 | 53.62 | |
| ATOM | 273 | SD | MET | | 180 | −30.652 | 81.376 | −58.824 | 1.00 | 51.13 | |
| ATOM | 274 | CE | MET | | 180 | −32.386 | 81.183 | −59.120 | 1.00 | 54.25 | |
| ATOM | 275 | C | MET | | 180 | −28.594 | 84.765 | −60.873 | 1.00 | 58.77 | |
| ATOM | 276 | O | MET | | 180 | −27.975 | 84.039 | −61.648 | 1.00 | 60.13 | |
| ATOM | 277 | N | LYS | | 181 | −28.013 | 85.535 | −59.958 | 1.00 | 58.72 | |
| ATOM | 278 | CA | LYS | | 181 | −26.572 | 85.603 | −59.788 | 1.00 | 59.60 | |
| ATOM | 279 | CB | LYS | | 181 | −26.187 | 87.005 | −59.319 | 1.00 | 61.96 | |
| ATOM | 280 | CG | LYS | | 181 | −24.713 | 87.331 | −59.448 | 1.00 | 65.96 | |
| ATOM | 281 | CD | LYS | | 181 | −24.435 | 88.802 | −59.115 | 1.00 | 69.70 | |
| ATOM | 282 | CE | LYS | | 181 | −22.954 | 89.170 | −59.317 | 1.00 | 69.92 | |
| ATOM | 283 | NZ | LYS | | 181 | −22.638 | 90.606 | −59.012 | 1.00 | 70.79 | |
| ATOM | 284 | C | LYS | | 181 | −26.167 | 84.566 | −58.742 | 1.00 | 59.64 | |
| ATOM | 285 | O | LYS | | 181 | −26.525 | 84.689 | −57.569 | 1.00 | 59.52 | |
| ATOM | 286 | N | ILE | | 182 | −25.426 | 83.546 | −59.170 | 1.00 | 59.53 | |
| ATOM | 287 | CA | ILE | | 182 | −24.982 | 82.469 | −58.280 | 1.00 | 59.64 | |
| ATOM | 288 | CR | ILE | | 182 | −25.313 | 81.081 | −58.895 | 1.00 | 58.23 | |
| ATOM | 289 | CG2 | ILE | | 182 | −24.940 | 79.975 | −57.933 | 1.00 | 55.70 | |
| ATOM | 290 | CG1 | ILE | | 182 | −26.806 | 80.996 | −59.218 | 1.00 | 57.10 | |
| ATOM | 291 | CD1 | ILE | | 182 | −27.180 | 79.776 | −60.026 | 1.00 | 56.15 | |
| ATOM | 292 | C | ILE | | 182 | −23.479 | 82.530 | −57.987 | 1.00 | 60.78 | |
| ATOM | 293 | O | ILE | | 182 | −22.656 | 82.252 | −58.858 | 1.00 | 61.27 | |
| ATOM | 294 | N | LEU | | 183 | −23.137 | 82.866 | −56.747 | 1.00 | 62.07 | |
| ATOM | 295 | CA | LEU | | 183 | −21.744 | 82.985 | −56.317 | 1.00 | 64.98 | |
| ATOM | 296 | CB | LEU | | 183 | −21.571 | 84.278 | −55.519 | 1.00 | 63.44 | |
| ATOM | 297 | CG | LEU | | 183 | −21.966 | 85.574 | −56.201 | 1.00 | 61.23 | |
| ATOM | 298 | CD1 | LEU | | 183 | −21.741 | 86.707 | −55.230 | 1.00 | 58.89 | |
| ATOM | 299 | CD2 | LEU | | 183 | −21.161 | 85.760 | −57.475 | 1.00 | 59.81 | |
| ATOM | 300 | C | LEU | | 183 | −21.195 | 81.833 | −55.461 | 1.00 | 67.22 | |
| ATOM | 301 | O | LEU | | 183 | −21.814 | 81.444 | −54.459 | 1.00 | 66.64 | |
| ATOM | 302 | N | ARG | | 184 | −20.025 | 81.313 | −55.839 | 1.00 | 69.55 | |
| ATOM | 303 | CA | ARG | | 184 | −19.387 | 80.236 | −55.074 | 1.00 | 73.02 | |
| ATOM | 304 | CB | ARG | | 184 | −18.086 | 79.774 | −55.757 | 1.00 | 74.15 | |
| ATOM | 305 | CG | ARG | | 184 | −18.166 | 79.378 | −57.246 | 1.00 | 77.69 | |
| ATOM | 306 | CD | ARG | | 184 | −18.491 | 77.888 | −57.458 | 1.00 | 79.90 | |
| ATOM | 307 | NE | ARG | | 184 | −18.364 | 77.439 | −58.855 | 1.00 | 82.50 | |
| ATOM | 308 | CZ | ARG | | 184 | −18.975 | 77.994 | −59.912 | 1.00 | 84.72 | |
| ATOM | 309 | NH1 | ARG | | 184 | −19.777 | 79.049 | −59.774 | 1.00 | 85.63 | |
| ATOM | 310 | NH2 | ARG | | 184 | −18.800 | 77.477 | −61.123 | 1.00 | 84.83 | |
| ATOM | 311 | C | ARG | | 184 | −19.033 | 80.810 | −53.686 | 1.00 | 74.65 | |
| ATOM | 312 | O | ARG | | 184 | −18.373 | 81.839 | −53.597 | 1.00 | 74.63 | |
| ATOM | 313 | N | LYS | | 185 | −19.480 | 80.176 | −52.605 | 1.00 | 76.64 | |
| ATOM | 314 | CA | LYS | | 185 | −19.134 | 80.674 | −51.271 | 1.00 | 78.41 | |
| ATOM | 315 | CB | LYS | | 185 | −19.851 | 79.882 | −50.169 | 1.00 | 75.30 | |
| ATOM | 316 | CG | LYS | | 185 | −21.260 | 80.331 | −49.833 | 1.00 | 73.34 | |
| ATOM | 317 | CD | LYS | | 185 | −21.873 | 79.438 | −48.754 | 1.00 | 71.48 | |
| ATOM | 318 | CE | LYS | | 185 | −23.277 | 79.893 | −48.349 | 1.00 | 70.10 | |
| ATOM | 319 | NZ | LYS | | 185 | −23.934 | 78.985 | −47.355 | 1.00 | 67.84 | |
| ATOM | 320 | C | LYS | | 185 | −17.629 | 80.460 | −51.135 | 1.00 | 81.73 | |
| ATOM | 321 | O | LYS | | 185 | −16.971 | 81.033 | −50.265 | 1.00 | 82.47 | |
| ATOM | 322 | N | GLU | | 186 | −17.101 | 79.627 | −52.025 | 1.00 | 85.05 | |
| ATOM | 323 | CA | GLU | | 186 | −15.687 | 79.267 | −52.059 | 1.00 | 88.41 | |
| ATOM | 324 | CB | GLU | | 186 | −15.537 | 77.990 | −52.887 | 1.00 | 89.94 | |
| ATOM | 325 | CG | GLU | | 186 | −14.140 | 77.401 | −52.970 | 1.00 | 92.22 | |
| ATOM | 326 | CD | GLU | | 186 | −14.165 | 75.989 | −53.535 | 1.00 | 93.16 | |
| ATOM | 327 | OE1 | GLU | | 186 | −14.633 | 75.811 | −54.684 | 1.00 | 92.42 | |
| ATOM | 328 | OE2 | GLU | | 186 | −13.728 | 75.057 | −52.822 | 1.00 | 94.10 | |
| ATOM | 329 | C | GLU | | 186 | −14.795 | 80.377 | −52.621 | 1.00 | 90.23 | |
| ATOM | 330 | O | GLU | | 186 | −13.661 | 80.556 | −52.183 | 1.00 | 91.51 | |
| ATOM | 331 | N | VAL | | 187 | −15.305 | 81.116 | −53.597 | 1.00 | 91.46 | |
| ATOM | 332 | CA | VAL | | 187 | −14.546 | 82.207 | −54.192 | 1.00 | 92.72 | |
| ATOM | 333 | CB | VAL | | 187 | −15.153 | 82.614 | −55.561 | 1.00 | 93.10 | |
| ATOM | 334 | CG1 | VAL | | 187 | −14.672 | 84.015 | −55.976 | 1.00 | 92.86 | |
| ATOM | 335 | CG2 | VAL | | 187 | −14.774 | 81.570 | −56.616 | 1.00 | 92.89 | |
| ATOM | 336 | C | VAL | | 187 | −14.515 | 83.409 | −53.248 | 1.00 | 93.50 | |
| ATOM | 337 | O | VAL | | 187 | −13.470 | 84.021 | −53.043 | 1.00 | 93.75 | |
| ATOM | 338 | N | ILE | | 188 | −15.660 | 83.744 | −52.668 | 1.00 | 94.77 | |
| ATOM | 339 | CA | ILE | | 188 | −15.729 | 84.867 | −51.744 | 1.00 | 96.38 | |
| ATOM | 340 | CB | ILE | | 188 | −17.106 | 85.583 | −51.817 | 1.00 | 97.58 | |
| ATOM | 341 | CG2 | ILE | | 188 | −18.202 | 84.653 | −51.323 | 1.00 | 97.46 | |
| ATOM | 342 | CG1 | ILE | | 188 | −17.093 | 86.853 | −50.956 | 1.00 | 98.72 | |
| ATOM | 343 | CD1 | ILE | | 188 | −18.323 | 87.755 | −51.143 | 1.00 | 99.63 | |
| ATOM | 344 | C | ILE | | 188 | −15.515 | 84.350 | −50.327 | 1.00 | 96.39 | |
| ATOM | 345 | O | ILE | | 188 | −14.548 | 84.716 | −49.665 | 1.00 | 97.59 | |
| ATOM | 346 | N | ALA | | 198 | −20.044 | 91.656 | −49.943 | 1.00 | 111.27 | |
| ATOM | 347 | CA | ALA | | 198 | −20.100 | 92.787 | −49.020 | 1.00 | 111.39 | |
| ATOM | 348 | CB | ALA | | 198 | −18.767 | 92.924 | −48.288 | 1.00 | 111.17 | |
| ATOM | 349 | C | ALA | | 198 | −20.433 | 94.091 | −49.745 | 1.00 | 111.44 | |
| ATOM | 350 | O | ALA | | 198 | −21.557 | 94.586 | −49.677 | 1.00 | 110.50 | |
| ATOM | 351 | N | ALA | | 199 | −19.439 | 94.644 | −50.435 | 1.00 | 112.22 | |
| ATOM | 352 | CA | ALA | | 199 | −19.611 | 95.887 | −51.184 | 1.00 | 112.15 | |
| ATOM | 353 | CB | ALA | | 199 | −18.274 | 96.324 | −51.806 | 1.00 | 111.66 | |
| ATOM | 354 | C | ALA | | 199 | −20.659 | 95.689 | −52.272 | 1.00 | 111.80 | |
| ATOM | 355 | O | ALA | | 199 | −21.257 | 96.652 | −52.750 | 1.00 | 111.96 | |
| ATOM | 356 | N | ALA | | 200 | −20.875 | 94.434 | −52.659 | 1.00 | 111.30 | |
| ATOM | 357 | CA | ALA | | 200 | −21.861 | 94.095 | −53.687 | 1.00 | 110.31 | |
| ATOM | 358 | CB | ALA | | 200 | −21.427 | 92.823 | −54.430 | 1.00 | 109.64 | |
| ATOM | 359 | C | ALA | | 200 | −23.235 | 93.892 | −53.038 | 1.00 | 109.45 | |
| ATOM | 360 | O | ALA | | 200 | −24.164 | 94.681 | −53.235 | 1.00 | 109.02 | |
| ATOM | 361 | N | ALA | | 201 | −23.352 | 92.828 | −52.254 | 1.00 | 108.62 | |
| ATOM | 362 | CA | ALA | | 201 | −24.597 | 92.525 | −51.568 | 1.00 | 107.31 | |
| ATOM | 363 | CB | ALA | | 201 | −24.485 | 91.182 | −50.856 | 1.00 | 107.26 | |
| ATOM | 364 | C | ALA | | 201 | −24.870 | 93.635 | −50.560 | 1.00 | 106.33 | |
| ATOM | 365 | O | ALA | | 201 | −24.094 | 94.586 | −50.459 | 1.00 | 106.70 | |
| ATOM | 366 | N | ALA | | 202 | −25.974 | 93.498 | −49.823 | 1.00 | 104.62 | |
| ATOM | 367 | CA | ALA | | 202 | −26.393 | 94.460 | −48.798 | 1.00 | 102.14 | |
| ATOM | 368 | CB | ALA | | 202 | −25.391 | 94.457 | −47.640 | 1.00 | 102.24 | |
| ATOM | 369 | C | ALA | | 202 | −26.565 | 95.876 | −49.346 | 1.00 | 100.18 | |
| ATOM | 370 | O | ALA | | 202 | −27.661 | 96.436 | −49.323 | 1.00 | 99.14 | |
| ATOM | 371 | N | ALA | | 203 | −25.467 | 96.443 | −49.831 | 1.00 | 98.53 | |
| ATOM | 372 | CA | ALA | | 203 | −25.448 | 97.783 | −50.399 | 1.00 | 96.44 | |
| ATOM | 373 | CB | ALA | | 203 | −24.049 | 98.102 | −50.933 | 1.00 | 96.12 | |
| ATOM | 374 | C | ALA | | 203 | −26.467 | 97.881 | −51.522 | 1.00 | 95.16 | |
| ATOM | 375 | O | ALA | | 203 | −27.372 | 98.713 | −51.473 | 1.00 | 94.87 | |
| ATOM | 376 | N | ALA | | 204 | −26.311 | 97.015 | −52.524 | 1.00 | 93.35 | |
| ATOM | 377 | CA | ALA | | 204 | −27.189 | 96.983 | −53.689 | 1.00 | 90.99 | |
| ATOM | 378 | CB | ALA | | 204 | −26.498 | 96.249 | −54.826 | 1.00 | 90.77 | |
| ATOM | 379 | C | ALA | | 204 | −28.546 | 96.349 | −53.410 | 1.00 | 89.70 | |
| ATOM | 380 | O | ALA | | 204 | −29.471 | 96.485 | −54.201 | 1.00 | 89.55 | |
| ATOM | 381 | N | ALA | | 205 | −28.668 | 95.653 | −52.288 | 1.00 | 88.45 | |
| ATOM | 382 | CA | ALA | | 205 | −29.929 | 95.009 | −51.940 | 1.00 | 86.63 | |
| ATOM | 383 | CB | ALA | | 205 | −29.695 | 93.931 | −50.892 | 1.00 | 87.44 | |
| ATOM | 384 | C | ALA | | 205 | −30.945 | 96.027 | −51.427 | 1.00 | 85.01 | |
| ATOM | 385 | O | ALA | | 205 | −32.140 | 95.909 | −51.701 | 1.00 | 85.23 | |
| ATOM | 386 | N | ALA | | 206 | −30.476 | 97.021 | −50.677 | 1.00 | 82.62 | |
| ATOM | 387 | CA | ALA | | 206 | −31.373 | 98.044 | −50.149 | 1.00 | 79.56 | |
| ATOM | 388 | CB | ALA | | 206 | −31.006 | 98.380 | −48.696 | 1.00 | 78.86 | |
| ATOM | 389 | C | ALA | | 206 | −31.389 | 99.317 | −51.014 | 1.00 | 77.67 | |
| ATOM | 390 | O | ALA | | 206 | −32.217 | 100.210 | −50.782 | 1.00 | 77.42 | |
| ATOM | 391 | N | THR | | 207 | −30.491 | 99.413 | −52.003 | 1.00 | 74.84 | |
| ATOM | 392 | CA | THR | | 207 | −30.485 | 100.589 | −52.887 | 1.00 | 71.87 | |
| ATOM | 393 | CB | THR | | 207 | −29.097 | 100.925 | −53.484 | 1.00 | 72.74 | |
| ATOM | 394 | OG1 | THR | | 207 | −28.555 | 99.752 | −54.106 | 1.00 | 73.95 | |
| ATOM | 395 | CG2 | THR | | 207 | −28.149 | 101.488 | −52.408 | 1.00 | 71.32 | |
| ATOM | 396 | C | THR | | 207 | −31.444 | 100.377 | −54.052 | 1.00 | 68.91 | |
| ATOM | 397 | O | THR | | 207 | −31.238 | 99.527 | −54.919 | 1.00 | 68.62 | |
| ATOM | 398 | N | ARG | | 208 | −32.495 | 101.183 | −54.035 | 1.00 | 66.01 | |
| ATOM | 399 | CA | ARG | | 208 | −33.569 | 101.181 | −55.009 | 1.00 | 62.69 | |
| ATOM | 400 | CB | ARG | | 208 | −34.880 | 101.401 | −54.251 | 1.00 | 65.69 | |
| ATOM | 401 | CG | ARG | | 208 | −36.133 | 101.250 | −55.073 | 1.00 | 70.98 | |
| ATOM | 402 | CD | ARG | | 208 | −36.384 | 99.777 | −55.385 | 1.00 | 74.29 | |
| ATOM | 403 | NE | ARG | | 208 | −37.662 | 99.572 | −56.060 | 1.00 | 76.66 | |
| ATOM | 404 | CZ | ARG | | 208 | −38.209 | 98.380 | −56.273 | 1.00 | 78.00 | |
| ATOM | 405 | NH1 | ARG | | 208 | −37.589 | 97.277 | −55.863 | 1.00 | 78.03 | |
| ATOM | 406 | NH2 | ARG | | 208 | −39.381 | 98.294 | −56.889 | 1.00 | 79.20 | |
| ATOM | 407 | C | ARG | | 208 | −33.323 | 102.351 | −55.963 | 1.00 | 57.98 | |
| ATOM | 408 | O | ARG | | 208 | −32.701 | 103.330 | −55.570 | 1.00 | 59.11 | |
| ATOM | 409 | N | HIS | | 209 | −33.789 | 102.251 | −57.205 | 1.00 | 51.68 | |
| ATOM | 410 | CA | HIS | | 209 | −33.643 | 103.345 | −58.162 | 1.00 | 46.72 | |
| ATOM | 411 | CB | HIS | | 209 | −32.200 | 103.778 | −58.280 | 1.00 | 43.33 | |
| ATOM | 412 | CG | HIS | | 209 | −32.018 | 105.063 | −59.026 | 1.00 | 42.75 | |
| ATOM | 413 | CD2 | HIS | | 209 | −31.578 | 106.278 | −58.616 | 1.00 | 41.65 | |
| ATOM | 414 | ND1 | HIS | | 209 | −32.254 | 105.179 | −60.380 | 1.00 | 42.43 | |
| ATOM | 415 | CE1 | HIS | | 209 | −31.959 | 106.407 | −60.774 | 1.00 | 41.53 | |
| ATOM | 416 | NE2 | HIS | | 209 | −31.546 | 107.094 | −59.723 | 1.00 | 41.40 | |
| ATOM | 417 | C | HIS | | 209 | −34.177 | 103.007 | −59.542 | 1.00 | 45.78 | |
| ATOM | 418 | O | HIS | | 209 | −33.909 | 101.942 | −60.080 | 1.00 | 46.54 | |
| ATOM | 419 | N | PRO | | 210 | −34.962 | 103.919 | −60.137 | 1.00 | 45.04 | |
| ATOM | 420 | CD | PRO | | 210 | −35.199 | 105.284 | −59.639 | 1.00 | 44.80 | |
| ATOM | 421 | CA | PRO | | 210 | −35.559 | 103.748 | −61.465 | 1.00 | 43.86 | |
| ATOM | 422 | CB | PRO | | 210 | −35.790 | 105.174 | −61.923 | 1.00 | 42.99 | |
| ATOM | 423 | CG | PRO | | 210 | −36.110 | 105.860 | −60.687 | 1.00 | 43.79 | |
| ATOM | 424 | C | PRO | | 210 | −34.676 | 103.024 | −62.453 | 1.00 | 43.47 | |
| ATOM | 425 | O | PRO | | 210 | −35.125 | 102.104 | −63.119 | 1.00 | 43.96 | |
| ATOM | 426 | N | PHE | | 211 | −33.421 | 103.458 | −62.531 | 1.00 | 42.97 | |
| ATOM | 427 | CA | PHE | | 211 | −32.459 | 102.920 | −63.487 | 1.00 | 43.73 | |
| ATOM | 428 | CB | PHE | | 211 | −31.731 | 104.080 | −64.166 | 1.00 | 43.15 | |
| ATOM | 429 | CG | PHE | | 211 | −32.644 | 105.180 | −64.592 | 1.00 | 43.15 | |
| ATOM | 430 | CD1 | PHE | | 211 | −33.798 | 104.898 | −65.327 | 1.00 | 43.74 | |
| ATOM | 431 | CD2 | PHE | | 211 | −32.405 | 106.490 | −64.206 | 1.00 | 43.08 | |
| ATOM | 432 | CE1 | PHE | | 211 | −34.699 | 105.908 | −65.660 | 1.00 | 40.39 | |
| ATOM | 433 | CE2 | PHE | | 211 | −33.310 | 107.501 | −64.538 | 1.00 | 40.15 | |
| ATOM | 434 | CZ | PHE | | 211 | −34.449 | 107.197 | −65.262 | 1.00 | 38.77 | |
| ATOM | 435 | C | PHE | | 211 | −31.431 | 101.905 | −62.991 | 1.00 | 44.53 | |
| ATOM | 436 | O | PHE | | 211 | −30.522 | 101.540 | −63.736 | 1.00 | 46.25 | |
| ATOM | 437 | N | LEU | | 212 | −31.543 | 101.483 | −61.735 | 1.00 | 44.20 | |
| ATOM | 438 | CA | LEU | | 212 | −30.653 | 100.464 | −61.193 | 1.00 | 42.38 | |
| ATOM | 439 | CB | LEU | | 212 | −30.135 | 100.831 | −59.807 | 1.00 | 39.21 | |
| ATOM | 440 | CG | LEU | | 212 | −29.022 | 101.872 | −59.692 | 1.00 | 39.56 | |
| ATOM | 441 | CD1 | LEU | | 212 | −28.523 | 101.885 | −58.262 | 1.00 | 39.36 | |
| ATOM | 442 | CD2 | LEU | | 212 | −27.860 | 101.545 | −60.604 | 1.00 | 39.12 | |
| ATOM | 443 | C | LEU | | 212 | −31.489 | 99.202 | −61.088 | 1.00 | 43.32 | |
| ATOM | 444 | O | LEU | | 212 | −32.662 | 99.248 | −60.701 | 1.00 | 43.07 | |
| ATOM | 445 | N | THR | | 213 | −30.898 | 98.078 | −61.468 | 1.00 | 44.44 | |
| ATOM | 446 | CA | THR | | 213 | −31.579 | 96.795 | −61.389 | 1.00 | 45.26 | |
| ATOM | 447 | CB | THR | | 213 | −30.646 | 95.699 | −61.849 | 1.00 | 45.78 | |
| ATOM | 448 | OG1 | THR | | 213 | −29.792 | 96.220 | −62.866 | 1.00 | 49.66 | |
| ATOM | 449 | CG2 | THR | | 213 | −31.415 | 94.530 | −62.387 | 1.00 | 44.85 | |
| ATOM | 450 | C | THR | | 213 | −31.908 | 96.532 | −59.912 | 1.00 | 45.99 | |
| ATOM | 451 | O | THR | | 213 | −31.066 | 96.766 | −59.039 | 1.00 | 45.57 | |
| ATOM | 452 | N | ALA | | 214 | −33.112 | 96.043 | −59.625 | 1.00 | 46.07 | |
| ATOM | 453 | CA | ALA | | 214 | −33.486 | 95.757 | −58.236 | 1.00 | 46.22 | |
| ATOM | 454 | CB | ALA | | 214 | −34.947 | 96.059 | −58.009 | 1.00 | 43.78 | |
| ATOM | 455 | C | ALA | | 214 | −33.208 | 94.315 | −57.836 | 1.00 | 46.04 | |
| ATOM | 456 | O | ALA | | 214 | −33.315 | 93.391 | −58.645 | 1.00 | 44.74 | |
| ATOM | 457 | N | LEU | | 215 | −32.856 | 94.127 | −56.574 | 1.00 | 47.88 | |
| ATOM | 458 | CA | LEU | | 215 | −32.583 | 92.802 | −56.055 | 1.00 | 50.44 | |
| ATOM | 459 | CB | LEU | | 215 | −31.614 | 92.875 | −54.903 | 1.00 | 51.79 | |
| ATOM | 460 | CG | LEU | | 215 | −31.518 | 91.485 | −54.297 | 1.00 | 53.08 | |
| ATOM | 461 | CD1 | LEU | | 215 | −30.496 | 90.672 | −55.109 | 1.00 | 53.05 | |
| ATOM | 462 | CD2 | LEU | | 215 | −31.139 | 91.590 | −52.820 | 1.00 | 52.91 | |
| ATOM | 463 | C | LEU | | 215 | −33.871 | 92.279 | −55.509 | 1.00 | 51.40 | |
| ATOM | 464 | O | LEU | | 215 | −34.264 | 92.675 | −54.424 | 1.00 | 52.98 | |
| ATOM | 465 | N | LYS | | 216 | −34.513 | 91.371 | −56.223 | 1.00 | 53.23 | |
| ATOM | 466 | CA | LYS | | 216 | −35.790 | 90.841 | −55.770 | 1.00 | 55.65 | |
| ATOM | 467 | CB | LYS | | 216 | −36.493 | 90.144 | −56.925 | 1.00 | 55.96 | |
| ATOM | 468 | CG | LYS | | 216 | −37.897 | 89.721 | −56.602 | 1.00 | 58.54 | |
| ATOM | 469 | CD | LYS | | 216 | −38.681 | 90.848 | −55.952 | 1.00 | 59.83 | |
| ATOM | 470 | CE | LYS | | 216 | −40.184 | 90.539 | −55.985 | 1.00 | 63.13 | |
| ATOM | 471 | NZ | LYS | | 216 | −40.549 | 89.164 | −55.477 | 1.00 | 63.74 | |
| ATOM | 472 | C | LYS | | 216 | −35.734 | 89.917 | −54.546 | 1.00 | 57.23 | |
| ATOM | 473 | O | LYS | | 216 | −36.483 | 90.120 | −53.596 | 1.00 | 58.03 | |
| ATOM | 474 | N | TYR | | 217 | −34.870 | 88.903 | −54.569 | 1.00 | 59.20 | |
| ATOM | 475 | CA | TYR | | 217 | −34.722 | 87.975 | −53.443 | 1.00 | 60.12 | |
| ATOM | 476 | CB | TYR | | 217 | −35.318 | 86.607 | −53.735 | 1.00 | 61.60 | |
| ATOM | 477 | CG | TYR | | 217 | −36.750 | 86.557 | −54.161 | 1.00 | 63.87 | |
| ATOM | 478 | CD1 | TYR | | 217 | −37.119 | 85.794 | −55.264 | 1.00 | 63.97 | |
| ATOM | 479 | CE1 | TYR | | 217 | −38.438 | 85.681 | −55.654 | 1.00 | 66.51 | |
| ATOM | 480 | CD2 | TYR | | 217 | −37.744 | 87.218 | −53.448 | 1.00 | 65.03 | |
| ATOM | 481 | CE2 | TYR | | 217 | −39.087 | 87.114 | −53.833 | 1.00 | 66.62 | |
| ATOM | 482 | CZ | TYR | | 217 | −39.421 | 86.341 | −54.941 | 1.00 | 66.84 | |
| ATOM | 483 | OH | TYR | | 217 | −40.721 | 86.236 | −55.362 | 1.00 | 66.74 | |
| ATOM | 484 | C | TYR | | 217 | −33.245 | 87.725 | −53.217 | 1.00 | 60.58 | |
| ATOM | 485 | O | TYR | | 217 | −32.437 | 87.818 | −54.146 | 1.00 | 62.04 | |
| ATOM | 486 | N | ALA | | 218 | −32.893 | 87.382 | −51.987 | 1.00 | 59.36 | |
| ATOM | 487 | CA | ALA | | 218 | −31.510 | 87.069 | −51.669 | 1.00 | 59.06 | |
| ATOM | 488 | CB | ALA | | 218 | −30.836 | 88.249 | −50.982 | 1.00 | 59.17 | |
| ATOM | 489 | C | ALA | | 218 | −31.535 | 85.852 | −50.752 | 1.00 | 58.60 | |
| ATOM | 490 | O | ALA | | 218 | −32.074 | 85.910 | −49.642 | 1.00 | 58.70 | |
| ATOM | 491 | N | PHE | | 219 | −30.989 | 84.739 | −51.218 | 1.00 | 57.97 | |
| ATOM | 492 | CA | PHE | | 219 | −30.983 | 83.551 | −50.397 | 1.00 | 57.83 | |
| ATOM | 493 | CB | PHE | | 219 | −32.179 | 82.647 | −50.721 | 1.00 | 56.56 | |
| ATOM | 494 | CG | PHE | | 219 | −32.047 | 81.884 | −52.011 | 1.00 | 56.06 | |
| ATOM | 495 | CD1 | PHE | | 219 | −32.502 | 82.415 | −53.200 | 1.00 | 57.08 | |
| ATOM | 496 | CD2 | PHE | | 219 | −31.479 | 80.625 | −52.033 | 1.00 | 55.89 | |
| ATOM | 497 | CE1 | PHE | | 219 | −32.396 | 81.703 | −54.390 | 1.00 | 56.64 | |
| ATOM | 498 | CE2 | PHE | | 219 | −31.373 | 79.923 | −53.209 | 1.00 | 55.25 | |
| ATOM | 499 | CZ | PHE | | 219 | −31.835 | 80.465 | −54.388 | 1.00 | 55.98 | |
| ATOM | 500 | C | PHE | | 219 | −29.706 | 82.784 | −50.581 | 1.00 | 58.69 | |
| ATOM | 501 | O | PHE | | 219 | −28.958 | 83.027 | −51.521 | 1.00 | 59.00 | |
| ATOM | 502 | N | GLN | | 220 | −29.450 | 81.852 | −49.677 | 1.00 | 60.19 | |
| ATOM | 503 | CA | GLN | | 220 | −28.246 | 81.054 | −49.795 | 1.00 | 62.39 | |
| ATOM | 504 | CB | GLN | | 220 | −27.206 | 81.489 | −48.767 | 1.00 | 64.75 | |
| ATOM | 505 | CG | GLN | | 220 | −27.666 | 81.361 | −47.328 | 1.00 | 68.63 | |
| ATOM | 506 | CD | GLN | | 220 | −26.492 | 81.319 | −46.366 | 1.00 | 71.92 | |
| ATOM | 507 | OE1 | GLN | | 220 | −25.490 | 82.023 | −46.563 | 1.00 | 73.88 | |
| ATOM | 508 | NE2 | GLN | | 220 | −26.607 | 80.501 | −45.312 | 1.00 | 72.13 | |
| ATOM | 509 | C | GLN | | 220 | −28.469 | 79.550 | −49.661 | 1.00 | 62.03 | |
| ATOM | 510 | O | GLN | | 220 | −29.420 | 79.078 | −49.027 | 1.00 | 62.34 | |
| ATOM | 511 | N | THR | | 221 | −27.562 | 78.814 | −50.285 | 1.00 | 60.95 | |
| ATOM | 512 | CA | THR | | 221 | −27.568 | 77.381 | −50.247 | 1.00 | 59.41 | |
| ATOM | 513 | CB | THR | | 221 | −27.254 | 76.834 | −51.636 | 1.00 | 59.17 | |
| ATOM | 514 | OG1 | THR | | 221 | −26.002 | 77.361 | −52.088 | 1.00 | 58.38 | |
| ATOM | 515 | CG2 | THR | | 221 | −28.340 | 77.253 | −52.618 | 1.00 | 58.65 | |
| ATOM | 516 | C | THR | | 221 | −26.458 | 77.052 | −49.242 | 1.00 | 60.17 | |
| ATOM | 517 | O | THR | | 221 | −25.880 | 77.951 | −48.633 | 1.00 | 60.99 | |
| ATOM | 518 | N | HIS | | 222 | −26.156 | 75.781 | −49.039 | 1.00 | 60.76 | |
| ATOM | 519 | CA | HIS | | 222 | −25.102 | 75.453 | −48.087 | 1.00 | 61.77 | |
| ATOM | 520 | CB | HIS | | 222 | −25.298 | 74.008 | −47.584 | 1.00 | 60.58 | |
| ATOM | 521 | CG | HIS | | 222 | −24.067 | 73.162 | −47.656 | 1.00 | 56.94 | |
| ATOM | 522 | CD2 | HIS | | 222 | −23.278 | 72.649 | −46.681 | 1.00 | 55.38 | |
| ATOM | 523 | ND1 | HIS | | 222 | −23.517 | 72.754 | −48.851 | 1.00 | 55.90 | |
| ATOM | 524 | CE1 | HIS | | 222 | −22.440 | 72.024 | −48.612 | 1.00 | 55.82 | |
| ATOM | 525 | NE2 | HIS | | 222 | −22.275 | 71.946 | −47.302 | 1.00 | 55.59 | |
| ATOM | 526 | C | HIS | | 222 | −23.710 | 75.639 | −48.714 | 1.00 | 61.88 | |
| ATOM | 527 | O | HIS | | 222 | −22.682 | 75.609 | −48.035 | 1.00 | 60.39 | |
| ATOM | 528 | N | ASP | | 223 | −23.699 | 75.897 | −50.009 | 1.00 | 62.01 | |
| ATOM | 529 | CA | ASP | | 223 | −22.461 | 76.016 | −50.739 | 1.00 | 62.36 | |
| ATOM | 530 | CB | ASP | | 223 | −22.353 | 74.737 | −51.572 | 1.00 | 62.94 | |
| ATOM | 531 | CG | ASP | | 223 | −21.436 | 74.868 | −52.737 | 1.00 | 63.63 | |
| ATOM | 532 | OD1 | ASP | | 223 | −21.921 | 74.826 | −53.894 | 1.00 | 63.16 | |
| ATOM | 533 | OD2 | ASP | | 223 | −20.226 | 75.009 | −52.481 | 1.00 | 65.54 | |
| ATOM | 534 | C | ASP | | 223 | −22.353 | 77.273 | −51.615 | 1.00 | 62.99 | |
| ATOM | 535 | O | ASP | | 223 | −21.313 | 77.513 | −52.242 | 1.00 | 63.19 | |
| ATOM | 536 | N | ARG | | 224 | −23.413 | 78.082 | −51.659 | 1.00 | 62.05 | |
| ATOM | 537 | CA | ARG | | 224 | −23.407 | 79.272 | −52.509 | 1.00 | 60.21 | |
| ATOM | 538 | CB | ARG | | 224 | −23.827 | 78.911 | −53.939 | 1.00 | 58.56 | |
| ATOM | 539 | CG | ARG | | 224 | −23.235 | 77.626 | −54.449 | 1.00 | 57.87 | |
| ATOM | 540 | CD | ARG | | 224 | −23.091 | 77.648 | −55.926 | 1.00 | 56.29 | |
| ATOM | 541 | NE | ARG | | 224 | −22.321 | 76.512 | −56.389 | 1.00 | 55.08 | |
| ATOM | 542 | CZ | ARG | | 224 | −21.966 | 76.333 | −57.656 | 1.00 | 57.33 | |
| ATOM | 543 | NH1 | ARG | | 224 | −22.311 | 77.217 | −58.587 | 1.00 | 57.43 | |
| ATOM | 544 | NH2 | ARG | | 224 | −21.263 | 75.264 | −58.001 | 1.00 | 59.13 | |
| ATOM | 545 | C | ARG | | 224 | −24.311 | 80.392 | −52.053 | 1.00 | 60.29 | |
| ATOM | 546 | O | ARG | | 224 | −25.144 | 80.222 | −51.157 | 1.00 | 59.45 | |
| ATOM | 547 | N | LEU | | 225 | −24.137 | 81.538 | −52.706 | 1.00 | 60.51 | |
| ATOM | 548 | CA | LEU | | 225 | −24.947 | 82.721 | −52.450 | 1.00 | 61.55 | |
| ATOM | 549 | CB | LEU | | 225 | −24.071 | 83.916 | −52.058 | 1.00 | 63.02 | |
| ATOM | 550 | CG | LEU | | 225 | −23.521 | 83.920 | −50.623 | 1.00 | 64.17 | |
| ATOM | 551 | CD1 | LEU | | 225 | −22.911 | 85.286 | −50.320 | 1.00 | 63.56 | |
| ATOM | 552 | CD2 | LEU | | 225 | −24.649 | 83.614 | −49.622 | 1.00 | 64.04 | |
| ATOM | 553 | C | LEU | | 225 | −25.724 | 83.027 | −53.731 | 1.00 | 61.30 | |
| ATOM | 554 | O | LEU | | 225 | −25.155 | 83.010 | −54.827 | 1.00 | 61.77 | |
| ATOM | 555 | N | CYS | | 226 | −27.021 | 83.295 | −53.586 | 1.00 | 59.68 | |
| ATOM | 556 | CA | CYS | | 226 | −27.877 | 83.578 | −54.725 | 1.00 | 58.60 | |
| ATOM | 557 | CB | CYS | | 226 | −28.920 | 82.476 | −54.870 | 1.00 | 59.17 | |
| ATOM | 558 | SG | CYS | | 226 | −28.211 | 80.907 | −55.333 | 1.00 | 59.56 | |
| ATOM | 559 | C | CYS | | 226 | −28.595 | 84.909 | −54.645 | 1.00 | 58.03 | |
| ATOM | 560 | O | CYS | | 226 | −29.187 | 85.241 | −53.623 | 1.00 | 58.16 | |
| ATOM | 561 | N | PHE | | 227 | −28.556 | 85.667 | −55.736 | 1.00 | 57.36 | |
| ATOM | 562 | CA | PHE | | 227 | −29.249 | 86.950 | −55.791 | 1.00 | 56.31 | |
| ATOM | 563 | CB | PHE | | 227 | −28.249 | 88.097 | −55.884 | 1.00 | 57.09 | |
| ATOM | 564 | CG | PHE | | 227 | −27.318 | 88.189 | −54.699 | 1.00 | 55.74 | |
| ATOM | 565 | CD1 | PHE | | 227 | −26.040 | 87.654 | −54.762 | 1.00 | 54.75 | |
| ATOM | 566 | CD2 | PHE | | 227 | −27.730 | 88.807 | −53.522 | 1.00 | 55.28 | |
| ATOM | 567 | CE1 | PHE | | 227 | −25.187 | 87.736 | −53.676 | 1.00 | 55.81 | |
| ATOM | 568 | CE2 | PHE | | 227 | −26.887 | 88.895 | −52.434 | 1.00 | 55.49 | |
| ATOM | 569 | CZ | PHE | | 227 | −25.613 | 88.361 | −52.507 | 1.00 | 56.68 | |
| ATOM | 570 | C | PHE | | 227 | −30.166 | 86.952 | −57.002 | 1.00 | 56.05 | |
| ATOM | 571 | O | PHE | | 227 | −29.719 | 87.127 | −58.129 | 1.00 | 56.45 | |
| ATOM | 572 | N | VAL | | 228 | −31.448 | 86.710 | −56.757 | 1.00 | 55.64 | |
| ATOM | 573 | CA | VAL | | 228 | −32.458 | 86.680 | −57.797 | 1.00 | 55.27 | |
| ATOM | 574 | CB | VAL | | 228 | −33.716 | 85.999 | −57.288 | 1.00 | 55.66 | |
| ATOM | 575 | CG1 | VAL | | 228 | −34.839 | 86.140 | −58.313 | 1.00 | 55.60 | |
| ATOM | 576 | CG2 | VAL | | 228 | −33.422 | 84.551 | −56.967 | 1.00 | 54.36 | |
| ATOM | 577 | C | VAL | | 228 | −32.812 | 88.112 | −58.092 | 1.00 | 56.00 | |
| ATOM | 578 | O | VAL | | 228 | −33.421 | 88.765 | −57.257 | 1.00 | 55.89 | |
| ATOM | 579 | N | MET | | 229 | −32.454 | 88.611 | −59.270 | 1.00 | 57.58 | |
| ATOM | 580 | CA | MET | | 229 | −32.750 | 90.008 | −59.594 | 1.00 | 58.37 | |
| ATOM | 581 | CB | MET | | 229 | −31.475 | 90.793 | −59.652 | 1.00 | 59.57 | |
| ATOM | 582 | CG | MET | | 229 | −30.745 | 90.725 | −58.372 | 1.00 | 63.19 | |
| ATOM | 583 | SD | MET | | 229 | −29.202 | 91.470 | −58.688 | 1.00 | 70.23 | |
| ATOM | 584 | CE | MET | | 229 | −28.769 | 90.676 | −60.338 | 1.00 | 67.31 | |
| ATOM | 585 | C | MET | | 229 | −33.531 | 90.292 | −60.844 | 1.00 | 57.66 | |
| ATOM | 586 | O | MET | | 229 | −33.457 | 89.547 | −61.812 | 1.00 | 58.68 | |
| ATOM | 587 | N | GLU | | 230 | −34.260 | 91.405 | −60.804 | 1.00 | 57.78 | |
| ATOM | 588 | CA | GLU | | 230 | −35.110 | 91.860 | −61.904 | 1.00 | 57.98 | |
| ATOM | 589 | CB | GLU | | 230 | −35.741 | 93.217 | −61.556 | 1.00 | 60.35 | |
| ATOM | 590 | CG | GLU | | 230 | −34.744 | 94.370 | −61.480 | 1.00 | 63.42 | |
| ATOM | 591 | CD | GLU | | 230 | −35.380 | 95.749 | −61.716 | 1.00 | 65.17 | |
| ATOM | 592 | OE1 | GLU | | 230 | −36.017 | 95.968 | −62.781 | 1.00 | 65.34 | |
| ATOM | 593 | OE2 | GLU | | 230 | −35.222 | 96.621 | −60.838 | 1.00 | 65.88 | |
| ATOM | 594 | C | GLU | | 230 | −34.395 | 91.965 | −63.251 | 1.00 | 56.10 | |
| ATOM | 595 | O | GLU | | 230 | −33.313 | 92.549 | −63.350 | 1.00 | 53.79 | |
| ATOM | 596 | N | TYR | | 231 | −35.025 | 91.410 | −64.288 | 1.00 | 55.38 | |
| ATOM | 597 | CA | TYR | | 231 | −34.449 | 91.424 | −65.624 | 1.00 | 56.06 | |
| ATOM | 598 | CB | TYR | | 231 | −34.368 | 90.000 | −66.158 | 1.00 | 57.59 | |
| ATOM | 599 | CG | TYR | | 231 | −33.509 | 89.855 | −67.393 | 1.00 | 58.75 | |
| ATOM | 600 | CD1 | TYR | | 231 | −32.154 | 90.220 | −67.373 | 1.00 | 59.60 | |
| ATOM | 601 | CE1 | TYR | | 231 | −31.340 | 90.052 | −68.505 | 1.00 | 61.01 | |
| ATOM | 602 | CD2 | TYR | | 231 | −34.037 | 89.323 | −68.575 | 1.00 | 58.59 | |
| ATOM | 603 | CE2 | TYR | | 231 | −33.239 | 89.153 | −69.708 | 1.00 | 60.96 | |
| ATOM | 604 | CZ | TYR | | 231 | −31.892 | 89.514 | −69.672 | 1.00 | 62.62 | |
| ATOM | 605 | OH | TYR | | 231 | −31.101 | 89.307 | −70.794 | 1.00 | 64.40 | |
| ATOM | 606 | C | TYR | | 231 | −35.190 | 92.293 | −66.642 | 1.00 | 56.11 | |
| ATOM | 607 | O | TYR | | 231 | −36.364 | 92.068 | −66.941 | 1.00 | 54.71 | |
| ATOM | 608 | N | ALA | | 232 | −34.477 | 93.280 | −67.181 | 1.00 | 56.15 | |
| ATOM | 609 | CA | ALA | | 232 | −35.021 | 94.192 | −68.185 | 1.00 | 55.56 | |
| ATOM | 610 | CB | ALA | | 232 | −34.036 | 95.301 | −68.448 | 1.00 | 54.61 | |
| ATOM | 611 | C | ALA | | 232 | −35.229 | 93.394 | −69.449 | 1.00 | 55.59 | |
| ATOM | 612 | O | ALA | | 232 | −34.376 | 92.587 | −69.795 | 1.00 | 55.66 | |
| ATOM | 613 | N | ASN | | 233 | −36.336 | 93.605 | −70.153 | 1.00 | 56.51 | |
| ATOM | 614 | CA | ASN | | 233 | −36.545 | 92.836 | −71.377 | 1.00 | 57.65 | |
| ATOM | 615 | CB | ASN | | 233 | −37.793 | 91.943 | −71.274 | 1.00 | 59.23 | |
| ATOM | 616 | CG | ASN | | 233 | −39.009 | 92.691 | −70.824 | 1.00 | 60.87 | |
| ATOM | 617 | OD1 | ASN | | 233 | −39.677 | 93.344 | −71.622 | 1.00 | 62.75 | |
| ATOM | 618 | ND2 | ASN | | 233 | −39.301 | 92.621 | −69.528 | 1.00 | 63.07 | |
| ATOM | 619 | C | ASN | | 233 | −36.576 | 93.664 | −72.643 | 1.00 | 56.89 | |
| ATOM | 620 | O | ASN | | 233 | −37.218 | 93.306 | −73.620 | 1.00 | 57.27 | |
| ATOM | 621 | N | GLY | | 234 | −35.851 | 94.770 | −72.623 | 1.00 | 56.49 | |
| ATOM | 622 | CA | GLY | | 234 | −35.768 | 95.613 | −73.794 | 1.00 | 56.62 | |
| ATOM | 623 | C | GLY | | 234 | −34.437 | 95.431 | −74.505 | 1.00 | 56.75 | |
| ATOM | 624 | O | GLY | | 234 | −34.007 | 96.309 | −75.255 | 1.00 | 57.72 | |
| ATOM | 625 | N | GLY | | 235 | −33.774 | 94.299 | −74.274 | 1.00 | 55.93 | |
| ATOM | 626 | CA | GLY | | 235 | −32.499 | 94.049 | −74.926 | 1.00 | 55.04 | |
| ATOM | 627 | C | GLY | | 235 | −31.347 | 94.831 | −74.329 | 1.00 | 54.89 | |
| ATOM | 628 | O | GLY | | 235 | −31.528 | 95.587 | −73.377 | 1.00 | 54.91 | |
| ATOM | 629 | N | GLU | | 236 | −30.154 | 94.670 | −74.885 | 1.00 | 55.24 | |
| ATOM | 630 | CA | GLU | | 236 | −29.004 | 95.368 | −74.328 | 1.00 | 55.07 | |
| ATOM | 631 | CB | GLU | | 236 | −27.852 | 94.404 | −74.112 | 1.00 | 56.18 | |
| ATOM | 632 | CG | GLU | | 236 | −27.842 | 93.269 | −75.080 | 1.00 | 59.38 | |
| ATOM | 633 | CD | GLU | | 236 | −28.404 | 92.007 | −74.471 | 1.00 | 62.42 | |
| ATOM | 634 | OE1 | GLU | | 236 | −29.574 | 91.653 | −74.791 | 1.00 | 62.53 | |
| ATOM | 635 | OE2 | GLU | | 236 | −27.665 | 91.380 | −73.662 | 1.00 | 62.54 | |
| ATOM | 636 | C | GLU | | 236 | −28.534 | 96.510 | −75.175 | 1.00 | 54.13 | |
| ATOM | 637 | O | GLU | | 236 | −28.433 | 96.382 | −76.387 | 1.00 | 54.23 | |
| ATOM | 638 | N | LEU | | 237 | −28.234 | 97.628 | −74.529 | 1.00 | 53.71 | |
| ATOM | 639 | CA | LEU | | 237 | −27.783 | 98.788 | −75.265 | 1.00 | 54.93 | |
| ATOM | 640 | CB | LEU | | 237 | −27.252 | 99.857 | −74.329 | 1.00 | 54.04 | |
| ATOM | 641 | CG | LEU | | 237 | −28.248 | 100.998 | −74.338 | 1.00 | 53.95 | |
| ATOM | 642 | CD1 | LEU | | 237 | −27.709 | 102.156 | −73.522 | 1.00 | 54.66 | |
| ATOM | 643 | CD2 | LEU | | 237 | −28.500 | 101.409 | −75.784 | 1.00 | 53.81 | |
| ATOM | 644 | C | LEU | | 237 | −26.725 | 98.420 | −76.291 | 1.00 | 56.21 | |
| ATOM | 645 | O | LEU | | 237 | −26.824 | 98.812 | −77.456 | 1.00 | 56.21 | |
| ATOM | 646 | N | PHE | | 238 | −25.717 | 97.667 | −75.864 | 1.00 | 58.45 | |
| ATOM | 647 | CA | PHE | | 238 | −24.687 | 97.238 | −76.792 | 1.00 | 59.89 | |
| ATOM | 648 | CB | PHE | | 238 | −23.792 | 96.165 | −76.162 | 1.00 | 63.73 | |
| ATOM | 649 | CG | PHE | | 238 | −23.289 | 95.200 | −77.160 | 1.00 | 68.93 | |
| ATOM | 650 | CD1 | PHE | | 238 | −22.333 | 95.601 | −78.100 | 1.00 | 71.68 | |
| ATOM | 651 | CD2 | PHE | | 238 | −23.908 | 93.955 | −77.305 | 1.00 | 70.77 | |
| ATOM | 652 | CE1 | PHE | | 238 | −22.003 | 94.782 | −79.193 | 1.00 | 74.10 | |
| ATOM | 653 | CE2 | PHE | | 238 | −23.596 | 93.120 | −78.386 | 1.00 | 73.70 | |
| ATOM | 654 | CZ | PHE | | 238 | −22.639 | 93.536 | −79.340 | 1.00 | 74.73 | |
| ATOM | 655 | C | PHE | | 238 | −25.374 | 96.661 | −78.066 | 1.00 | 59.02 | |
| ATOM | 656 | O | PHE | | 238 | −24.983 | 96.969 | −79.201 | 1.00 | 58.51 | |
| ATOM | 657 | N | PHE | | 239 | −26.387 | 95.822 | −77.866 | 1.00 | 57.17 | |
| ATOM | 658 | CA | PHE | | 239 | −27.116 | 95.238 | −78.980 | 1.00 | 56.51 | |
| ATOM | 659 | CB | PHE | | 239 | −28.324 | 94.445 | −78.471 | 1.00 | 58.38 | |
| ATOM | 660 | CG | PHE | | 239 | −29.260 | 93.985 | −79.566 | 1.00 | 58.40 | |
| ATOM | 661 | CD1 | PHE | | 239 | −28.957 | 92.862 | −80.342 | 1.00 | 59.15 | |
| ATOM | 662 | CD2 | PHE | | 239 | −30.430 | 94.683 | −79.830 | 1.00 | 57.90 | |
| ATOM | 663 | CE1 | PHE | | 239 | −29.812 | 92.443 | −81.369 | 1.00 | 59.43 | |
| ATOM | 664 | CE2 | PHE | | 239 | −31.293 | 94.270 | −80.859 | 1.00 | 59.40 | |
| ATOM | 665 | CZ | PHE | | 239 | −30.984 | 93.151 | −81.628 | 1.00 | 59.17 | |
| ATOM | 666 | C | PHE | | 239 | −27.618 | 96.329 | −79.920 | 1.00 | 55.27 | |
| ATOM | 667 | O | PHE | | 239 | −27.177 | 96.426 | −81.060 | 1.00 | 55.80 | |
| ATOM | 668 | N | HIS | | 240 | −28.558 | 97.134 | −79.420 | 1.00 | 53.43 | |
| ATOM | 669 | CA | HIS | | 240 | −29.171 | 98.228 | −80.172 | 1.00 | 51.94 | |
| ATOM | 670 | CB | HIS | | 240 | −30.103 | 99.043 | −79.257 | 1.00 | 50.17 | |
| ATOM | 671 | CG | HIS | | 240 | −31.250 | 98.258 | −78.691 | 1.00 | 46.78 | |
| ATOM | 672 | CD2 | HIS | | 240 | −31.564 | 97.936 | −77.412 | 1.00 | 46.08 | |
| ATOM | 673 | ND1 | HIS | | 240 | −32.233 | 97.700 | −79.478 | 1.00 | 45.57 | |
| ATOM | 674 | CE1 | HIS | | 240 | −33.103 | 97.066 | −78.710 | 1.00 | 45.18 | |
| ATOM | 675 | NE2 | HIS | | 240 | −32.720 | 97.194 | −77.454 | 1.00 | 45.22 | |
| ATOM | 676 | C | HIS | | 240 | −28.135 | 99.165 | −80.815 | 1.00 | 51.95 | |
| ATOM | 677 | O | HIS | | 240 | −28.259 | 99.520 | −81.993 | 1.00 | 52.45 | |
| ATOM | 678 | N | LEU | | 241 | −27.123 | 99.573 | −80.052 | 1.00 | 50.66 | |
| ATOM | 679 | CA | LEU | | 241 | −26.090 | 100.439 | −80.595 | 1.00 | 51.66 | |
| ATOM | 680 | CB | LEU | | 241 | −25.000 | 100.715 | −79.555 | 1.00 | 48.76 | |
| ATOM | 681 | CG | LEU | | 241 | −23.880 | 101.590 | −80.112 | 1.00 | 46.97 | |
| ATOM | 682 | CD1 | LEU | | 241 | −24.521 | 102.823 | −80.697 | 1.00 | 48.02 | |
| ATOM | 683 | CD2 | LEU | | 241 | −22.854 | 101.969 | −79.049 | 1.00 | 47.08 | |
| ATOM | 684 | C | LEU | | 241 | −25.442 | 99.829 | −81.842 | 1.00 | 54.73 | |
| ATOM | 685 | O | LEU | | 241 | −25.232 | 100.516 | −82.847 | 1.00 | 55.64 | |
| ATOM | 686 | N | SER | | 242 | −25.119 | 98.544 | −81.802 | 1.00 | 56.62 | |
| ATOM | 687 | CA | SER | | 242 | −24.494 | 97.949 | −82.968 | 1.00 | 59.11 | |
| ATOM | 688 | CB | SER | | 242 | −23.920 | 96.580 | −82.632 | 1.00 | 58.85 | |
| ATOM | 689 | OG | SER | | 242 | −24.802 | 95.876 | −81.797 | 1.00 | 62.13 | |
| ATOM | 690 | C | SER | | 242 | −25.434 | 97.848 | −84.166 | 1.00 | 61.10 | |
| ATOM | 691 | O | SER | | 242 | −24.978 | 97.767 | −85.310 | 1.00 | 61.61 | |
| ATOM | 692 | N | ARG | | 243 | −26.740 | 97.861 | −83.923 | 1.00 | 62.64 | |
| ATOM | 693 | CA | ARG | | 243 | −27.687 | 97.787 | −85.030 | 1.00 | 64.71 | |
| ATOM | 694 | CB | ARG | | 243 | −29.046 | 97.264 | −84.563 | 1.00 | 66.18 | |
| ATOM | 695 | CG | ARG | | 243 | −29.021 | 95.813 | −84.068 | 1.00 | 70.06 | |
| ATOM | 696 | CD | ARG | | 243 | −30.424 | 95.242 | −84.090 | 1.00 | 73.49 | |
| ATOM | 697 | NE | ARG | | 243 | −30.942 | 95.238 | −85.455 | 1.00 | 78.42 | |
| ATOM | 698 | CZ | ARG | | 243 | −32.214 | 95.033 | −85.783 | 1.00 | 79.64 | |
| ATOM | 699 | NH1 | ARG | | 243 | −33.117 | 94.813 | −84.831 | 1.00 | 81.30 | |
| ATOM | 700 | NH2 | ARG | | 243 | −32.581 | 95.047 | −87.063 | 1.00 | 79.34 | |
| ATOM | 701 | C | ARG | | 243 | −27.862 | 99.167 | −85.631 | 1.00 | 64.74 | |
| ATOM | 702 | O | ARG | | 243 | −28.608 | 99.353 | −86.587 | 1.00 | 65.12 | |
| ATOM | 703 | N | GLU | | 244 | −27.153 | 100.136 | −85.072 | 1.00 | 64.07 | |
| ATOM | 704 | CA | GLU | | 244 | −27.264 | 101.498 | −85.550 | 1.00 | 63.44 | |
| ATOM | 705 | CB | GLU | | 244 | −28.160 | 102.304 | −84.623 | 1.00 | 63.93 | |
| ATOM | 706 | CG | GLU | | 244 | −29.603 | 101.920 | −84.755 | 1.00 | 65.33 | |
| ATOM | 707 | CD | GLU | | 244 | −30.505 | 102.799 | −83.939 | 1.00 | 67.27 | |
| ATOM | 708 | OE1 | GLU | | 244 | −30.580 | 102.588 | −82.705 | 1.00 | 67.76 | |
| ATOM | 709 | OE2 | GLU | | 244 | −31.128 | 103.708 | −84.539 | 1.00 | 68.46 | |
| ATOM | 710 | C | GLU | | 244 | −25.958 | 102.216 | −85.701 | 1.00 | 62.23 | |
| ATOM | 711 | O | GLU | | 244 | −25.961 | 103.408 | −86.000 | 1.00 | 63.17 | |
| ATOM | 712 | N | ARG | | 245 | −24.854 | 101.501 | −85.489 | 1.00 | 60.84 | |
| ATOM | 713 | CA | ARG | | 245 | −23.502 | 102.066 | −85.605 | 1.00 | 58.99 | |
| ATOM | 714 | CB | ARG | | 245 | −23.236 | 102.486 | −87.069 | 1.00 | 58.85 | |
| ATOM | 715 | CG | ARG | | 245 | −21.846 | 103.050 | −87.362 | 1.00 | 60.59 | |
| ATOM | 716 | CD | ARG | | 245 | −20.726 | 102.072 | −87.020 | 1.00 | 63.59 | |
| ATOM | 717 | NE | ARG | | 245 | −19.936 | 101.635 | −88.180 | 1.00 | 67.49 | |
| ATOM | 718 | CZ | ARG | | 245 | −20.165 | 100.534 | −88.902 | 1.00 | 68.65 | |
| ATOM | 719 | NH1 | ARG | | 245 | −21.173 | 99.722 | −88.608 | 1.00 | 70.75 | |
| ATOM | 720 | NH2 | ARG | | 245 | −19.368 | 100.229 | −89.920 | 1.00 | 69.78 | |
| ATOM | 721 | C | ARG | | 245 | −23.255 | 103.236 | −84.626 | 1.00 | 56.71 | |
| ATOM | 722 | O | ARG | | 245 | −22.246 | 103.234 | −83.900 | 1.00 | 56.07 | |
| ATOM | 723 | N | VAL | | 246 | −24.175 | 104.207 | −84.607 | 1.00 | 53.86 | |
| ATOM | 724 | CA | VAL | | 246 | −24.095 | 105.363 | −83.727 | 1.00 | 53.06 | |
| ATOM | 725 | CB | VAL | | 246 | −23.407 | 106.571 | −84.401 | 1.00 | 54.68 | |
| ATOM | 726 | CG1 | VAL | | 246 | −22.668 | 107.425 | −83.338 | 1.00 | 52.61 | |
| ATOM | 727 | CG2 | VAL | | 246 | −22.446 | 106.097 | −85.495 | 1.00 | 56.84 | |
| ATOM | 728 | C | VAL | | 246 | −25.483 | 105.819 | −83.324 | 1.00 | 52.80 | |
| ATOM | 729 | O | VAL | | 246 | −26.440 | 105.584 | −84.033 | 1.00 | 53.52 | |
| ATOM | 730 | N | PHE | | 247 | −25.584 | 106.462 | −82.166 | 1.00 | 53.24 | |
| ATOM | 731 | CA | PHE | | 247 | −26.844 | 106.994 | −81.661 | 1.00 | 52.35 | |
| ATOM | 732 | CB | PHE | | 247 | −26.976 | 106.756 | −80.154 | 1.00 | 51.14 | |
| ATOM | 733 | CG | PHE | | 247 | −27.508 | 105.395 | −79.777 | 1.00 | 51.57 | |
| ATOM | 734 | CD1 | PHE | | 247 | −28.268 | 104.649 | −80.667 | 1.00 | 50.67 | |
| ATOM | 735 | CD2 | PHE | | 247 | −27.293 | 104.888 | −78.490 | 1.00 | 51.25 | |
| ATOM | 736 | CE1 | PHE | | 247 | −28.805 | 103.426 | −80.279 | 1.00 | 51.34 | |
| ATOM | 737 | CE2 | PHE | | 247 | −27.822 | 103.669 | −78.100 | 1.00 | 50.31 | |
| ATOM | 738 | CZ | PHE | | 247 | −28.580 | 102.935 | −78.989 | 1.00 | 50.52 | |
| ATOM | 739 | C | PHE | | 247 | −26.820 | 108.509 | −81.908 | 1.00 | 54.13 | |
| ATOM | 740 | O | PHE | | 247 | −25.791 | 109.073 | −82.300 | 1.00 | 53.35 | |
| ATOM | 741 | N | THR | | 248 | −27.940 | 109.178 | −81.660 | 1.00 | 55.42 | |
| ATOM | 742 | CA | THR | | 248 | −28.012 | 110.617 | −81.866 | 1.00 | 56.42 | |
| ATOM | 743 | CB | THR | | 248 | −29.418 | 111.048 | −82.231 | 1.00 | 58.86 | |
| ATOM | 744 | OG1 | THR | | 248 | −29.704 | 110.625 | −83.573 | 1.00 | 60.69 | |
| ATOM | 745 | CG2 | THR | | 248 | −29.554 | 112.568 | −82.109 | 1.00 | 59.43 | |
| ATOM | 746 | C | THR | | 248 | −27.608 | 111.354 | −80.619 | 1.00 | 56.27 | |
| ATOM | 747 | O | THR | | 248 | −27.994 | 110.956 | −79.518 | 1.00 | 57.63 | |
| ATOM | 748 | N | GLU | | 249 | −26.863 | 112.443 | −80.790 | 1.00 | 55.72 | |
| ATOM | 749 | CA | GLU | | 249 | −26.395 | 113.208 | −79.648 | 1.00 | 56.33 | |
| ATOM | 750 | CB | GLU | | 249 | −25.773 | 114.523 | −80.092 | 1.00 | 57.14 | |
| ATOM | 751 | CG | GLU | | 249 | −24.423 | 114.380 | −80.750 | 1.00 | 58.64 | |
| ATOM | 752 | CD | GLU | | 249 | −23.690 | 115.710 | −80.817 | 1.00 | 60.38 | |
| ATOM | 753 | OE1 | GLU | | 249 | −23.628 | 116.384 | −79.776 | 1.00 | 62.11 | |
| ATOM | 754 | OE2 | GLU | | 249 | −23.172 | 116.087 | −81.886 | 1.00 | 61.08 | |
| ATOM | 755 | C | GLU | | 249 | −27.486 | 113.468 | −78.612 | 1.00 | 56.82 | |
| ATOM | 756 | O | GLU | | 249 | −27.213 | 113.962 | −77.517 | 1.00 | 57.02 | |
| ATOM | 757 | N | GLU | | 250 | −28.721 | 113.144 | −78.960 | 1.00 | 57.10 | |
| ATOM | 758 | CA | GLU | | 250 | −29.814 | 113.318 | −78.030 | 1.00 | 59.34 | |
| ATOM | 759 | CB | GLU | | 250 | −31.072 | 113.823 | −78.730 | 1.00 | 62.10 | |
| ATOM | 760 | CG | GLU | | 250 | −31.276 | 115.328 | −78.624 | 1.00 | 65.60 | |
| ATOM | 761 | CD | GLU | | 250 | −31.330 | 115.805 | −77.183 | 1.00 | 67.27 | |
| ATOM | 762 | OE1 | GLU | | 250 | −30.255 | 115.869 | −76.530 | 1.00 | 68.52 | |
| ATOM | 763 | OE2 | GLU | | 250 | −32.451 | 116.105 | −76.709 | 1.00 | 66.68 | |
| ATOM | 764 | C | GLU | | 250 | −30.095 | 111.974 | −77.414 | 1.00 | 58.83 | |
| ATOM | 765 | O | GLU | | 250 | −30.177 | 111.852 | −76.187 | 1.00 | 60.92 | |
| ATOM | 766 | N | ARG | | 251 | −30.233 | 110.966 | −78.268 | 1.00 | 56.44 | |
| ATOM | 767 | CA | ARG | | 251 | −30.501 | 109.619 | −77.797 | 1.00 | 54.72 | |
| ATOM | 768 | CB | ARG | | 251 | −30.474 | 108.631 | −78.948 | 1.00 | 54.18 | |
| ATOM | 769 | CG | ARG | | 251 | −31.385 | 107.465 | −78.709 | 1.00 | 54.07 | |
| ATOM | 770 | CD | ARG | | 251 | −30.722 | 106.182 | −79.096 | 1.00 | 53.40 | |
| ATOM | 771 | NE | ARG | | 251 | −31.487 | 105.437 | −80.082 | 1.00 | 53.76 | |
| ATOM | 772 | CZ | ARG | | 251 | −32.032 | 104.246 | −79.860 | 1.00 | 55.64 | |
| ATOM | 773 | NH1 | ARG | | 251 | −31.896 | 103.660 | −78.680 | 1.00 | 55.82 | |
| ATOM | 774 | NH2 | ARG | | 251 | −32.717 | 103.632 | −80.820 | 1.00 | 56.77 | |
| ATOM | 775 | C | ARG | | 251 | −29.407 | 109.264 | −76.800 | 1.00 | 53.82 | |
| ATOM | 776 | O | ARG | | 251 | −29.657 | 108.695 | −75.741 | 1.00 | 52.67 | |
| ATOM | 777 | N | ALA | | 252 | −28.182 | 109.609 | −77.148 | 1.00 | 52.30 | |
| ATOM | 778 | CA | ALA | | 252 | −27.093 | 109.340 | −76.247 | 1.00 | 52.96 | |
| ATOM | 779 | CB | ALA | | 252 | −25.782 | 109.939 | −76.782 | 1.00 | 54.05 | |
| ATOM | 780 | C | ALA | | 252 | −27.509 | 110.044 | −74.983 | 1.00 | 52.90 | |
| ATOM | 781 | O | ALA | | 252 | −27.744 | 109.410 | −73.967 | 1.00 | 54.24 | |
| ATOM | 782 | N | ALA | | 253 | −27.621 | 111.369 | −75.077 | 1.00 | 51.69 | |
| ATOM | 783 | CA | ALA | | 253 | −27.994 | 112.219 | −73.952 | 1.00 | 48.08 | |
| ATOM | 784 | CB | ALA | | 253 | −28.328 | 113.607 | −74.437 | 1.00 | 49.27 | |
| ATOM | 785 | C | ALA | | 253 | −29.146 | 111.654 | −73.134 | 1.00 | 46.00 | |
| ATOM | 786 | O | ALA | | 253 | −29.112 | 111.732 | −71.907 | 1.00 | 45.85 | |
| ATOM | 787 | N | PHE | | 254 | −30.160 | 111.100 | −73.791 | 1.00 | 42.03 | |
| ATOM | 788 | CA | PHE | | 254 | −31.266 | 110.529 | −73.042 | 1.00 | 41.60 | |
| ATOM | 789 | CB | PHE | | 254 | −32.210 | 109.778 | −73.941 | 1.00 | 44.00 | |
| ATOM | 790 | CG | PHE | | 254 | −33.337 | 109.124 | −73.209 | 1.00 | 48.04 | |
| ATOM | 791 | CD1 | PHE | | 254 | −34.345 | 109.886 | −72.638 | 1.00 | 49.95 | |
| ATOM | 792 | CD2 | PHE | | 254 | −33.422 | 107.736 | −73.137 | 1.00 | 50.49 | |
| ATOM | 793 | CE1 | PHE | | 254 | −35.439 | 109.271 | −72.006 | 1.00 | 51.92 | |
| ATOM | 794 | CE2 | PHE | | 254 | −34.503 | 107.102 | −72.511 | 1.00 | 51.03 | |
| ATOM | 795 | CZ | PHE | | 254 | −35.514 | 107.865 | −71.947 | 1.00 | 52.65 | |
| ATOM | 796 | C | PHE | | 254 | −30.674 | 109.557 | −72.045 | 1.00 | 41.01 | |
| ATOM | 797 | O | PHE | | 254 | −30.805 | 109.745 | −70.842 | 1.00 | 41.53 | |
| ATOM | 798 | N | TYR | | 255 | −29.999 | 108.529 | −72.560 | 1.00 | 40.14 | |
| ATOM | 799 | CA | TYR | | 255 | −29.332 | 107.515 | −71.747 | 1.00 | 36.58 | |
| ATOM | 800 | CB | TYR | | 255 | −28.635 | 106.511 | −72.638 | 1.00 | 37.23 | |
| ATOM | 801 | CG | TYR | | 255 | −29.594 | 105.830 | −73.563 | 1.00 | 39.49 | |
| ATOM | 802 | CD1 | TYR | | 255 | −30.778 | 105.317 | −73.082 | 1.00 | 38.77 | |
| ATOM | 803 | CE1 | TYR | | 255 | −31.680 | 104.716 | −73.932 | 1.00 | 42.51 | |
| ATOM | 804 | CD2 | TYR | | 255 | −29.332 | 105.725 | −74.930 | 1.00 | 40.34 | |
| ATOM | 805 | CE2 | TYR | | 255 | −30.227 | 105.132 | −75.789 | 1.00 | 40.12 | |
| ATOM | 806 | CZ | TYR | | 255 | −31.400 | 104.618 | −75.286 | 1.00 | 41.95 | |
| ATOM | 807 | OH | TYR | | 255 | −32.279 | 103.937 | −76.098 | 1.00 | 41.80 | |
| ATOM | 808 | C | TYR | | 255 | −28.316 | 108.106 | −70.797 | 1.00 | 34.60 | |
| ATOM | 809 | O | TYR | | 255 | −28.265 | 107.739 | −69.630 | 1.00 | 34.46 | |
| ATOM | 810 | N | GLY | | 256 | −27.510 | 109.024 | −71.303 | 1.00 | 33.03 | |
| ATOM | 811 | CA | GLY | | 256 | −26.502 | 109.646 | −70.481 | 1.00 | 34.98 | |
| ATOM | 812 | C | GLY | | 256 | −27.112 | 110.248 | −69.242 | 1.00 | 37.28 | |
| ATOM | 813 | O | GLY | | 256 | −26.511 | 110.221 | −68.157 | 1.00 | 36.19 | |
| ATOM | 814 | N | ALA | | 257 | −28.319 | 110.790 | −69.408 | 1.00 | 38.76 | |
| ATOM | 815 | CA | ALA | | 257 | −29.040 | 111.417 | −68.316 | 1.00 | 39.95 | |
| ATOM | 816 | CB | ALA | | 257 | −30.154 | 112.298 | −68.864 | 1.00 | 39.05 | |
| ATOM | 817 | C | ALA | | 257 | −29.601 | 110.395 | −67.310 | 1.00 | 40.20 | |
| ATOM | 818 | O | ALA | | 257 | −29.477 | 110.600 | −66.100 | 1.00 | 41.01 | |
| ATOM | 819 | N | GLU | | 258 | −30.211 | 109.308 | −67.774 | 1.00 | 38.83 | |
| ATOM | 820 | CA | GLU | | 258 | −30.723 | 108.348 | −66.802 | 1.00 | 41.08 | |
| ATOM | 821 | CB | GLU | | 258 | −31.524 | 107.215 | −67.474 | 1.00 | 40.29 | |
| ATOM | 822 | CG | GLU | | 258 | −32.521 | 107.739 | −68.512 | 1.00 | 42.23 | |
| ATOM | 823 | CD | GLU | | 258 | −33.741 | 106.852 | −68.742 | 1.00 | 46.18 | |
| ATOM | 824 | OE1 | GLU | | 258 | −33.585 | 105.619 | −68.855 | 1.00 | 46.61 | |
| ATOM | 825 | OE2 | GLU | | 258 | −34.876 | 107.391 | −68.839 | 1.00 | 49.04 | |
| ATOM | 826 | C | GLU | | 258 | −29.527 | 107.801 | −66.019 | 1.00 | 41.79 | |
| ATOM | 827 | O | GLU | | 258 | −29.601 | 107.600 | −64.798 | 1.00 | 41.76 | |
| ATOM | 828 | N | ILE | | 259 | −28.403 | 107.610 | −66.705 | 1.00 | 41.38 | |
| ATOM | 829 | CA | ILE | | 259 | −27.234 | 107.101 | −66.016 | 1.00 | 40.33 | |
| ATOM | 830 | CB | ILE | | 259 | −26.045 | 106.804 | −66.969 | 1.00 | 40.56 | |
| ATOM | 831 | CG2 | ILE | | 259 | −24.789 | 106.440 | −66.149 | 1.00 | 39.49 | |
| ATOM | 832 | CG1 | ILE | | 259 | −26.394 | 105.642 | −67.900 | 1.00 | 39.49 | |
| ATOM | 833 | CD1 | ILE | | 259 | −25.353 | 105.415 | −68.934 | 1.00 | 39.15 | |
| ATOM | 834 | C | ILE | | 259 | −26.776 | 108.096 | −64.978 | 1.00 | 40.77 | |
| ATOM | 835 | O | ILE | | 259 | −26.598 | 107.722 | −63.822 | 1.00 | 41.79 | |
| ATOM | 836 | N | VAL | | 260 | −26.583 | 109.355 | −65.387 | 1.00 | 40.24 | |
| ATOM | 837 | CA | VAL | | 260 | −26.125 | 110.415 | −64.468 | 1.00 | 37.59 | |
| ATOM | 838 | CB | VAL | | 260 | −26.185 | 111.823 | −65.129 | 1.00 | 34.92 | |
| ATOM | 839 | CG1 | VAL | | 260 | −25.857 | 112.883 | −64.114 | 1.00 | 32.77 | |
| ATOM | 840 | CG2 | VAL | | 260 | −25.176 | 111.918 | −66.253 | 1.00 | 34.85 | |
| ATOM | 841 | C | VAL | | 260 | −26.970 | 110.429 | −63.202 | 1.00 | 37.07 | |
| ATOM | 842 | O | VAL | | 260 | −26.455 | 110.626 | −62.102 | 1.00 | 34.02 | |
| ATOM | 843 | N | SER | | 261 | −28.271 | 110.194 | −63.377 | 1.00 | 36.64 | |
| ATOM | 844 | CA | SER | | 261 | −29.215 | 110.152 | −62.267 | 1.00 | 36.77 | |
| ATOM | 845 | CB | SER | | 261 | −30.636 | 109.969 | −62.785 | 1.00 | 37.47 | |
| ATOM | 846 | OG | SER | | 261 | −31.528 | 109.766 | −61.706 | 1.00 | 39.68 | |
| ATOM | 847 | C | SER | | 261 | −28.874 | 109.006 | −61.320 | 1.00 | 36.39 | |
| ATOM | 848 | O | SER | | 261 | −28.740 | 109.206 | −60.116 | 1.00 | 35.60 | |
| ATOM | 849 | N | ALA | | 262 | −28.736 | 107.809 | −61.882 | 1.00 | 35.91 | |
| ATOM | 850 | CA | ALA | | 262 | −28.384 | 106.621 | −61.110 | 1.00 | 34.56 | |
| ATOM | 851 | CB | ALA | | 262 | −28.198 | 105.437 | −62.035 | 1.00 | 33.00 | |
| ATOM | 852 | C | ALA | | 262 | −27.112 | 106.863 | −60.320 | 1.00 | 34.18 | |
| ATOM | 853 | O | ALA | | 262 | −27.017 | 106.524 | −59.156 | 1.00 | 35.06 | |
| ATOM | 854 | N | LEU | | 263 | −26.137 | 107.473 | −60.951 | 1.00 | 34.74 | |
| ATOM | 855 | CA | LEU | | 263 | −24.874 | 107.705 | −60.291 | 1.00 | 37.11 | |
| ATOM | 856 | CB | LEU | | 263 | −23.796 | 108.072 | −61.321 | 1.00 | 37.01 | |
| ATOM | 857 | CG | LEU | | 263 | −23.457 | 106.927 | −62.259 | 1.00 | 35.53 | |
| ATOM | 858 | CD1 | LEU | | 263 | −22.346 | 107.311 | −63.188 | 1.00 | 34.02 | |
| ATOM | 859 | CD2 | LEU | | 263 | −23.075 | 105.738 | −61.396 | 1.00 | 35.69 | |
| ATOM | 860 | C | LEU | | 263 | −24.921 | 108.754 | −59.219 | 1.00 | 38.74 | |
| ATOM | 861 | O | LEU | | 263 | −24.185 | 108.665 | −58.235 | 1.00 | 40.59 | |
| ATOM | 862 | N | GLU | | 264 | −25.750 | 109.772 | −59.410 | 1.00 | 41.02 | |
| ATOM | 863 | CA | GLU | | 264 | −25.840 | 110.807 | −58.393 | 1.00 | 42.25 | |
| ATOM | 864 | CB | GLU | | 264 | −26.791 | 111.903 | −58.801 | 1.00 | 43.68 | |
| ATOM | 865 | CG | GLU | | 264 | −27.085 | 112.811 | −57.647 | 1.00 | 48.37 | |
| ATOM | 866 | CD | GLU | | 264 | −27.950 | 113.972 | −58.033 | 1.00 | 52.87 | |
| ATOM | 867 | OE1 | GLU | | 264 | −29.130 | 113.757 | −58.408 | 1.00 | 55.43 | |
| ATOM | 868 | OE2 | GLU | | 264 | −27.436 | 115.107 | −57.963 | 1.00 | 56.01 | |
| ATOM | 869 | C | GLU | | 264 | −26.377 | 110.155 | −57.140 | 1.00 | 41.62 | |
| ATOM | 870 | O | GLU | | 264 | −25.903 | 110.405 | −56.036 | 1.00 | 39.43 | |
| ATOM | 871 | N | TYR | | 265 | −27.374 | 109.304 | −57.334 | 1.00 | 41.96 | |
| ATOM | 872 | CA | TYR | | 265 | −27.982 | 108.620 | −56.224 | 1.00 | 43.36 | |
| ATOM | 873 | CB | TYR | | 265 | −29.099 | 107.706 | −56.696 | 1.00 | 46.93 | |
| ATOM | 874 | CG | TYR | | 265 | −29.949 | 107.250 | −55.535 | 1.00 | 54.20 | |
| ATOM | 875 | CD1 | TYR | | 265 | −30.771 | 108.173 | −54.850 | 1.00 | 58.79 | |
| ATOM | 876 | CE1 | TYR | | 265 | −31.545 | 107.791 | −53.731 | 1.00 | 58.88 | |
| ATOM | 877 | CD2 | TYR | | 265 | −29.915 | 105.926 | −55.081 | 1.00 | 53.90 | |
| ATOM | 878 | CE2 | TYR | | 265 | −30.683 | 105.526 | −53.964 | 1.00 | 56.95 | |
| ATOM | 879 | CZ | TYR | | 265 | −31.498 | 106.466 | −53.288 | 1.00 | 58.96 | |
| ATOM | 880 | OH | TYR | | 265 | −32.256 | 106.102 | −52.170 | 1.00 | 58.88 | |
| ATOM | 881 | C | TYR | | 265 | −26.939 | 107.801 | −55.467 | 1.00 | 42.79 | |
| ATOM | 882 | O | TYR | | 265 | −26.759 | 107.978 | −54.252 | 1.00 | 41.77 | |
| ATOM | 883 | N | LEU | | 266 | −26.256 | 106.910 | −56.193 | 1.00 | 40.98 | |
| ATOM | 884 | CA | LEU | | 266 | −25.232 | 106.045 | −55.608 | 1.00 | 36.65 | |
| ATOM | 885 | CB | LEU | | 266 | −24.601 | 105.144 | −56.660 | 1.00 | 35.25 | |
| ATOM | 886 | CG | LEU | | 266 | −25.523 | 104.107 | −57.291 | 1.00 | 34.78 | |
| ATOM | 887 | CD1 | LEU | | 266 | −24.845 | 103.565 | −58.495 | 1.00 | 34.51 | |
| ATOM | 888 | CD2 | LEU | | 266 | −25.841 | 102.987 | −56.322 | 1.00 | 35.50 | |
| ATOM | 889 | C | LEU | | 266 | −24.146 | 106.834 | −54.936 | 1.00 | 35.02 | |
| ATOM | 890 | O | LEU | | 266 | −23.752 | 106.500 | −53.843 | 1.00 | 33.78 | |
| ATOM | 891 | N | HIS | | 267 | −23.655 | 107.885 | −55.569 | 1.00 | 34.64 | |
| ATOM | 892 | CA | HIS | | 267 | −22.602 | 108.639 | −54.921 | 1.00 | 35.63 | |
| ATOM | 893 | CB | HIS | | 267 | −21.964 | 109.646 | −55.875 | 1.00 | 35.65 | |
| ATOM | 894 | CG | HIS | | 267 | −21.273 | 109.027 | −57.046 | 1.00 | 36.53 | |
| ATOM | 895 | CD2 | HIS | | 267 | −21.221 | 107.748 | −57.478 | 1.00 | 37.02 | |
| ATOM | 896 | ND1 | HIS | | 267 | −20.546 | 109.766 | −57.951 | 1.00 | 37.83 | |
| ATOM | 897 | CE1 | HIS | | 267 | −20.078 | 108.967 | −58.894 | 1.00 | 37.83 | |
| ATOM | 898 | NE2 | HIS | | 267 | −20.472 | 107.738 | −58.631 | 1.00 | 37.12 | |
| ATOM | 899 | C | HIS | | 267 | −23.107 | 109.385 | −53.699 | 1.00 | 37.37 | |
| ATOM | 900 | O | HIS | | 267 | −22.321 | 109.787 | −52.862 | 1.00 | 37.40 | |
| ATOM | 901 | N | SER | | 268 | −24.410 | 109.612 | −53.603 | 1.00 | 40.64 | |
| ATOM | 902 | CA | SER | | 268 | −24.928 | 110.329 | −52.447 | 1.00 | 42.75 | |
| ATOM | 903 | CB | SER | | 268 | −26.351 | 110.833 | −52.695 | 1.00 | 43.40 | |
| ATOM | 904 | OG | SER | | 268 | −27.255 | 109.754 | −52.870 | 1.00 | 46.67 | |
| ATOM | 905 | C | SER | | 268 | −24.912 | 109.358 | −51.285 | 1.00 | 43.85 | |
| ATOM | 906 | O | SER | | 268 | −24.706 | 109.757 | −50.139 | 1.00 | 42.93 | |
| ATOM | 907 | N | ARG | | 269 | −25.142 | 108.085 | −51.597 | 1.00 | 45.20 | |
| ATOM | 908 | CA | ARG | | 269 | −25.122 | 107.028 | −50.597 | 1.00 | 47.42 | |
| ATOM | 909 | CB | ARG | | 269 | −25.911 | 105.812 | −51.074 | 1.00 | 49.68 | |
| ATOM | 910 | CG | ARG | | 269 | −27.399 | 106.019 | −51.078 | 1.00 | 55.46 | |
| ATOM | 911 | CD | ARG | | 269 | −27.854 | 106.411 | −49.687 | 1.00 | 58.44 | |
| ATOM | 912 | NE | ARG | | 269 | −29.232 | 106.886 | −49.656 | 1.00 | 61.49 | |
| ATOM | 913 | CZ | ARG | | 269 | −29.721 | 107.845 | −50.440 | 1.00 | 63.34 | |
| ATOM | 914 | NH1 | ARG | | 269 | −28.946 | 108.440 | −51.341 | 1.00 | 63.40 | |
| ATOM | 915 | NH2 | ARG | | 269 | −30.988 | 108.224 | −50.304 | 1.00 | 63.31 | |
| ATOM | 916 | C | ARG | | 269 | −23.675 | 106.603 | −50.358 | 1.00 | 47.89 | |
| ATOM | 917 | O | ARG | | 269 | −23.424 | 105.589 | −49.704 | 1.00 | 48.73 | |
| ATOM | 918 | N | ASP | | 270 | −22.731 | 107.360 | −50.916 | 1.00 | 46.60 | |
| ATOM | 919 | CA | ASP | | 270 | −21.320 | 107.059 | −50.753 | 1.00 | 47.20 | |
| ATOM | 920 | CB | ASP | | 270 | −20.955 | 107.159 | −49.270 | 1.00 | 51.45 | |
| ATOM | 921 | CG | ASP | | 270 | −19.463 | 107.258 | −49.054 | 1.00 | 56.02 | |
| ATOM | 922 | OD1 | ASP | | 270 | −18.735 | 106.794 | −49.952 | 1.00 | 59.22 | |
| ATOM | 923 | OD2 | ASP | | 270 | −19.003 | 107.786 | −48.014 | 1.00 | 58.08 | |
| ATOM | 924 | C | ASP | | 270 | −20.960 | 105.656 | −51.282 | 1.00 | 45.77 | |
| ATOM | 925 | O | ASP | | 270 | −20.291 | 104.890 | −50.598 | 1.00 | 47.78 | |
| ATOM | 926 | N | VAL | | 271 | −21.400 | 105.336 | −52.498 | 1.00 | 41.94 | |
| ATOM | 927 | CA | VAL | | 271 | −21.183 | 104.045 | −53.146 | 1.00 | 37.02 | |
| ATOM | 928 | CB | VAL | | 271 | −22.484 | 103.341 | −53.365 | 1.00 | 35.61 | |
| ATOM | 929 | CG1 | VAL | | 271 | −22.333 | 102.319 | −54.452 | 1.00 | 32.84 | |
| ATOM | 930 | CG2 | VAL | | 271 | −22.935 | 102.714 | −52.107 | 1.00 | 35.19 | |
| ATOM | 931 | C | VAL | | 271 | −20.615 | 104.242 | −54.528 | 1.00 | 38.27 | |
| ATOM | 932 | O | VAL | | 271 | −21.261 | 104.865 | −55.348 | 1.00 | 39.48 | |
| ATOM | 933 | N | VAL | | 272 | −19.441 | 103.684 | −54.818 | 1.00 | 39.56 | |
| ATOM | 934 | CA | VAL | | 272 | −18.835 | 103.864 | −56.141 | 1.00 | 38.29 | |
| ATOM | 935 | CB | VAL | | 272 | −17.290 | 103.882 | −56.096 | 1.00 | 37.80 | |
| ATOM | 936 | CG1 | VAL | | 272 | −16.751 | 103.924 | −57.510 | 1.00 | 37.97 | |
| ATOM | 937 | CG2 | VAL | | 272 | −16.785 | 105.095 | −55.326 | 1.00 | 34.59 | |
| ATOM | 938 | C | VAL | | 272 | −19.254 | 102.728 | −57.015 | 1.00 | 38.29 | |
| ATOM | 939 | O | VAL | | 272 | −18.962 | 101.592 | −56.712 | 1.00 | 40.46 | |
| ATOM | 940 | N | TYR | | 273 | −19.919 | 103.007 | −58.123 | 1.00 | 37.53 | |
| ATOM | 941 | CA | TYR | | 273 | −20.354 | 101.890 | −58.932 | 1.00 | 36.67 | |
| ATOM | 942 | CB | TYR | | 273 | −21.281 | 102.355 | −60.040 | 1.00 | 30.76 | |
| ATOM | 943 | CG | TYR | | 273 | −22.145 | 101.215 | −60.507 | 1.00 | 31.04 | |
| ATOM | 944 | CD1 | TYR | | 273 | −23.066 | 100.619 | −59.656 | 1.00 | 28.51 | |
| ATOM | 945 | CE1 | TYR | | 273 | −23.840 | 99.543 | −60.080 | 1.00 | 29.65 | |
| ATOM | 946 | CD2 | TYR | | 273 | −22.018 | 100.708 | −61.781 | 1.00 | 30.83 | |
| ATOM | 947 | CE2 | TYR | | 273 | −22.781 | 99.633 | −62.215 | 1.00 | 31.32 | |
| ATOM | 948 | CZ | TYR | | 273 | −23.687 | 99.052 | −61.368 | 1.00 | 31.70 | |
| ATOM | 949 | OH | TYR | | 273 | −24.420 | 97.962 | −61.824 | 1.00 | 32.78 | |
| ATOM | 950 | C | TYR | | 273 | −19.278 | 100.951 | −59.513 | 1.00 | 37.18 | |
| ATOM | 951 | O | TYR | | 273 | −19.470 | 99.740 | −59.488 | 1.00 | 37.07 | |
| ATOM | 952 | N | ARG | | 274 | −18.181 | 101.503 | −60.034 | 1.00 | 38.28 | |
| ATOM | 953 | CA | ARG | | 274 | −17.086 | 100.729 | −60.646 | 1.00 | 40.89 | |
| ATOM | 954 | CB | ARG | | 274 | −16.598 | 99.623 | −59.718 | 1.00 | 43.17 | |
| ATOM | 955 | CG | ARG | | 274 | −15.851 | 100.074 | −58.502 | 1.00 | 48.37 | |
| ATOM | 956 | CD | ARG | | 274 | −15.646 | 98.887 | −57.598 | 1.00 | 50.53 | |
| ATOM | 957 | NE | ARG | | 274 | −14.866 | 99.192 | −56.400 | 1.00 | 52.73 | |
| ATOM | 958 | CZ | ARG | | 274 | −13.646 | 98.710 | −56.176 | 1.00 | 54.09 | |
| ATOM | 959 | NH1 | ARG | | 274 | −13.065 | 97.912 | −57.083 | 1.00 | 53.66 | |
| ATOM | 960 | NH2 | ARG | | 274 | −13.027 | 98.986 | −55.029 | 1.00 | 52.88 | |
| ATOM | 961 | C | ARG | | 274 | −17.384 | 100.057 | −61.979 | 1.00 | 40.25 | |
| ATOM | 962 | O | ARG | | 274 | −16.485 | 99.809 | −62.744 | 1.00 | 40.06 | |
| ATOM | 963 | N | ASP | | 275 | −18.640 | 99.745 | −62.252 | 1.00 | 41.62 | |
| ATOM | 964 | CA | ASP | | 275 | −18.989 | 99.042 | −63.480 | 1.00 | 42.56 | |
| ATOM | 965 | CB | ASP | | 275 | −19.701 | 97.731 | −63.130 | 1.00 | 47.02 | |
| ATOM | 966 | CG | ASP | | 275 | −18.755 | 96.664 | −62.591 | 1.00 | 52.05 | |
| ATOM | 967 | OD1 | ASP | | 275 | −18.081 | 96.896 | −61.560 | 1.00 | 52.75 | |
| ATOM | 968 | OD2 | ASP | | 275 | −18.694 | 95.574 | −63.209 | 1.00 | 56.41 | |
| ATOM | 969 | C | ASP | | 275 | −19.838 | 99.776 | −64.513 | 1.00 | 41.87 | |
| ATOM | 970 | O | ASP | | 275 | −20.625 | 99.146 | −65.227 | 1.00 | 41.73 | |
| ATOM | 971 | N | ILE | | 276 | −19.711 | 101.089 | −64.619 | 1.00 | 40.20 | |
| ATOM | 972 | CA | ILE | | 276 | −20.506 | 101.760 | −65.632 | 1.00 | 38.96 | |
| ATOM | 973 | CB | ILE | | 276 | −20.609 | 103.265 | −65.377 | 1.00 | 38.40 | |
| ATOM | 974 | CG2 | ILE | | 276 | −21.168 | 103.971 | −66.601 | 1.00 | 35.75 | |
| ATOM | 975 | CG1 | ILE | | 276 | −21.486 | 103.514 | −64.140 | 1.00 | 36.92 | |
| ATOM | 976 | CD1 | ILE | | 276 | −22.840 | 102.866 | −64.216 | 1.00 | 35.84 | |
| ATOM | 977 | C | ILE | | 276 | −19.836 | 101.526 | −66.972 | 1.00 | 39.53 | |
| ATOM | 978 | O | ILE | | 276 | −18.831 | 102.153 | −67.287 | 1.00 | 39.09 | |
| ATOM | 979 | N | LYS | | 277 | −20.378 | 100.604 | −67.758 | 1.00 | 39.04 | |
| ATOM | 980 | CA | LYS | | 277 | −19.801 | 100.318 | −69.055 | 1.00 | 40.33 | |
| ATOM | 981 | CB | LYS | | 277 | −18.631 | 99.350 | −68.896 | 1.00 | 42.59 | |
| ATOM | 982 | CG | LYS | | 277 | −18.928 | 98.073 | −68.127 | 1.00 | 43.91 | |
| ATOM | 983 | CD | LYS | | 277 | −17.606 | 97.384 | −67.779 | 1.00 | 48.02 | |
| ATOM | 984 | CE | LYS | | 277 | −17.758 | 95.972 | −67.203 | 1.00 | 49.19 | |
| ATOM | 985 | NZ | LYS | | 277 | −17.436 | 94.948 | −68.257 | 1.00 | 49.33 | |
| ATOM | 986 | C | LYS | | 277 | −20.892 | 99.762 | −69.947 | 1.00 | 40.01 | |
| ATOM | 987 | O | LYS | | 277 | −21.810 | 99.117 | −69.467 | 1.00 | 41.30 | |
| ATOM | 988 | N | LEU | | 278 | −20.780 | 100.010 | −71.245 | 1.00 | 39.68 | |
| ATOM | 989 | CA | LEU | | 278 | −21.801 | 99.615 | −72.215 | 1.00 | 40.09 | |
| ATOM | 990 | CB | LEU | | 278 | −21.264 | 99.813 | −73.645 | 1.00 | 38.12 | |
| ATOM | 991 | CG | LEU | | 278 | −22.126 | 99.491 | −74.877 | 1.00 | 37.37 | |
| ATOM | 992 | CD1 | LEU | | 278 | −23.466 | 100.158 | −74.821 | 1.00 | 39.03 | |
| ATOM | 993 | CD2 | LEU | | 278 | −21.388 | 99.958 | −76.105 | 1.00 | 37.19 | |
| ATOM | 994 | C | LEU | | 278 | −22.407 | 98.236 | −72.085 | 1.00 | 40.69 | |
| ATOM | 995 | O | LEU | | 278 | −23.583 | 98.030 | −72.385 | 1.00 | 40.65 | |
| ATOM | 996 | N | GLU | | 279 | −21.606 | 97.292 | −71.626 | 1.00 | 42.71 | |
| ATOM | 997 | CA | GLU | | 279 | −22.054 | 95.910 | −71.488 | 1.00 | 44.42 | |
| ATOM | 998 | CB | GLU | | 279 | −20.832 | 94.996 | −71.352 | 1.00 | 47.35 | |
| ATOM | 999 | CG | GLU | | 279 | −19.664 | 95.380 | −72.283 | 1.00 | 51.14 | |
| ATOM | 1000 | CD | GLU | | 279 | −18.950 | 96.657 | −71.812 | 1.00 | 55.38 | |
| ATOM | 1001 | OE1 | GLU | | 279 | −18.934 | 96.864 | −70.574 | 1.00 | 57.08 | |
| ATOM | 1002 | OE2 | GLU | | 279 | −18.402 | 97.438 | −72.646 | 1.00 | 57.18 | |
| ATOM | 1003 | C | GLU | | 279 | −23.003 | 95.738 | −70.307 | 1.00 | 43.66 | |
| ATOM | 1004 | O | GLU | | 279 | −23.809 | 94.814 | −70.265 | 1.00 | 44.78 | |
| ATOM | 1005 | N | ASN | | 280 | −22.913 | 96.648 | −69.353 | 1.00 | 42.86 | |
| ATOM | 1006 | CA | ASN | | 280 | −23.771 | 96.605 | −68.187 | 1.00 | 43.61 | |
| ATOM | 1007 | CB | ASN | | 280 | −23.032 | 97.219 | −66.988 | 1.00 | 44.97 | |
| ATOM | 1008 | CG | ASN | | 280 | −22.262 | 96.195 | −66.159 | 1.00 | 46.33 | |
| ATOM | 1009 | OD1 | ASN | | 280 | −21.285 | 95.605 | −66.599 | 1.00 | 49.39 | |
| ATOM | 1010 | ND2 | ASN | | 280 | −22.706 | 96.000 | −64.933 | 1.00 | 49.29 | |
| ATOM | 1011 | C | ASN | | 280 | −25.079 | 97.391 | −68.434 | 1.00 | 43.20 | |
| ATOM | 1012 | O | ASN | | 280 | −25.821 | 97.641 | −67.498 | 1.00 | 44.95 | |
| ATOM | 1013 | N | LEU | | 281 | −25.374 | 97.783 | −69.672 | 1.00 | 40.99 | |
| ATOM | 1014 | CA | LEU | | 281 | −26.581 | 98.568 | −69.916 | 1.00 | 39.76 | |
| ATOM | 1015 | CB | LEU | | 281 | −26.222 | 99.902 | −70.585 | 1.00 | 36.50 | |
| ATOM | 1016 | CG | LEU | | 281 | −25.347 | 100.750 | −69.651 | 1.00 | 37.63 | |
| ATOM | 1017 | CD1 | LEU | | 281 | −24.798 | 101.997 | −70.320 | 1.00 | 34.48 | |
| ATOM | 1018 | CD2 | LEU | | 281 | −26.169 | 101.104 | −68.451 | 1.00 | 38.95 | |
| ATOM | 1019 | C | LEU | | 281 | −27.642 | 97.871 | −70.722 | 1.00 | 41.40 | |
| ATOM | 1020 | O | LEU | | 281 | −27.373 | 97.350 | −71.797 | 1.00 | 41.39 | |
| ATOM | 1021 | N | MET | | 282 | −28.858 | 97.857 | −70.183 | 1.00 | 43.20 | |
| ATOM | 1022 | CA | MET | | 282 | −30.014 | 97.256 | −70.851 | 1.00 | 44.72 | |
| ATOM | 1023 | CB | MET | | 282 | −30.422 | 95.996 | −70.123 | 1.00 | 43.76 | |
| ATOM | 1024 | CG | MET | | 282 | −29.466 | 94.890 | −70.334 | 1.00 | 46.63 | |
| ATOM | 1025 | SD | MET | | 282 | −30.291 | 93.309 | −70.088 | 1.00 | 50.69 | |
| ATOM | 1026 | CE | MET | | 282 | −30.382 | 93.294 | −68.257 | 1.00 | 51.55 | |
| ATOM | 1027 | C | MET | | 282 | −31.207 | 98.241 | −70.901 | 1.00 | 45.67 | |
| ATOM | 1028 | O | MET | | 282 | −31.231 | 99.245 | −70.177 | 1.00 | 46.72 | |
| ATOM | 1029 | N | LEU | | 283 | −32.180 | 97.979 | −71.765 | 1.00 | 44.26 | |
| ATOM | 1030 | CA | LEU | | 283 | −33.356 | 98.835 | −71.840 | 1.00 | 44.16 | |
| ATOM | 1031 | CB | LEU | | 283 | −33.662 | 99.237 | −73.291 | 1.00 | 41.73 | |
| ATOM | 1032 | CG | LEU | | 283 | −32.705 | 100.063 | −74.164 | 1.00 | 41.56 | |
| ATOM | 1033 | CD1 | LEU | | 283 | −33.409 | 100.385 | −75.477 | 1.00 | 39.31 | |
| ATOM | 1034 | CD2 | LEU | | 283 | −32.294 | 101.359 | −73.474 | 1.00 | 39.00 | |
| ATOM | 1035 | C | LEU | | 283 | −34.535 | 98.027 | −71.305 | 1.00 | 46.47 | |
| ATOM | 1036 | O | LEU | | 283 | −34.590 | 96.814 | −71.510 | 1.00 | 44.43 | |
| ATOM | 1037 | N | ASP | | 284 | −35.469 | 98.674 | −70.605 | 1.00 | 48.97 | |
| ATOM | 1038 | CA | ASP | | 284 | −36.650 | 97.937 | −70.140 | 1.00 | 51.54 | |
| ATOM | 1039 | CB | ASP | | 284 | −37.173 | 98.436 | −68.771 | 1.00 | 51.43 | |
| ATOM | 1040 | CG | ASP | | 284 | −37.637 | 99.898 | −68.773 | 1.00 | 51.84 | |
| ATOM | 1041 | OD1 | ASP | | 284 | −37.973 | 100.454 | −69.838 | 1.00 | 50.58 | |
| ATOM | 1042 | OD2 | ASP | | 284 | −37.683 | 100.483 | −67.665 | 1.00 | 52.12 | |
| ATOM | 1043 | C | ASP | | 284 | −37.764 | 98.005 | −71.210 | 1.00 | 53.72 | |
| ATOM | 1044 | O | ASP | | 284 | −37.669 | 98.774 | −72.178 | 1.00 | 52.20 | |
| ATOM | 1045 | N | LYS | | 285 | −38.798 | 97.183 | −71.048 | 1.00 | 55.54 | |
| ATOM | 1046 | CA | LYS | | 285 | −39.910 | 97.136 | −72.000 | 1.00 | 56.99 | |
| ATOM | 1047 | CB | LYS | | 285 | −41.120 | 96.496 | −71.330 | 1.00 | 57.83 | |
| ATOM | 1048 | CG | LYS | | 285 | −41.527 | 97.200 | −70.058 | 1.00 | 60.93 | |
| ATOM | 1049 | CD | LYS | | 285 | −42.642 | 96.446 | −69.371 | 1.00 | 63.52 | |
| ATOM | 1050 | CE | LYS | | 285 | −42.950 | 97.044 | −68.008 | 1.00 | 66.90 | |
| ATOM | 1051 | NZ | LYS | | 285 | −41.877 | 96.789 | −66.997 | 1.00 | 67.28 | |
| ATOM | 1052 | C | LYS | | 285 | −40.310 | 98.496 | −72.574 | 1.00 | 56.20 | |
| ATOM | 1053 | O | LYS | | 285 | −40.818 | 98.580 | −73.694 | 1.00 | 55.97 | |
| ATOM | 1054 | N | ASP | | 286 | −40.062 | 99.556 | −71.815 | 1.00 | 55.50 | |
| ATOM | 1055 | CA | ASP | | 286 | −40.426 | 100.894 | −72.249 | 1.00 | 56.10 | |
| ATOM | 1056 | CB | ASP | | 286 | −40.989 | 101.678 | −71.060 | 1.00 | 57.07 | |
| ATOM | 1057 | CG | ASP | | 286 | −42.273 | 101.076 | −70.513 | 1.00 | 58.89 | |
| ATOM | 1058 | OD1 | ASP | | 286 | −43.227 | 100.908 | −71.307 | 1.00 | 60.61 | |
| ATOM | 1059 | OD2 | ASP | | 286 | −42.328 | 100.782 | −69.291 | 1.00 | 58.93 | |
| ATOM | 1060 | C | ASP | | 286 | −39.345 | 101.734 | −72.929 | 1.00 | 55.50 | |
| ATOM | 1061 | O | ASP | | 286 | −39.646 | 102.770 | −73.502 | 1.00 | 56.75 | |
| ATOM | 1062 | N | GLY | | 287 | −38.090 | 101.316 | −72.863 | 1.00 | 54.81 | |
| ATOM | 1063 | CA | GLY | | 287 | −37.037 | 102.098 | −73.500 | 1.00 | 51.56 | |
| ATOM | 1064 | C | GLY | | 287 | −36.178 | 102.825 | −72.484 | 1.00 | 50.79 | |
| ATOM | 1065 | O | GLY | | 287 | −35.283 | 103.592 | −72.845 | 1.00 | 50.78 | |
| ATOM | 1066 | N | HIS | | 288 | −36.457 | 102.593 | −71.205 | 1.00 | 49.54 | |
| ATOM | 1067 | CA | HIS | | 288 | −35.700 | 103.211 | −70.117 | 1.00 | 48.54 | |
| ATOM | 1068 | CB | HIS | | 288 | −36.607 | 103.414 | −68.911 | 1.00 | 50.42 | |
| ATOM | 1069 | CG | HIS | | 288 | −37.503 | 104.594 | −69.035 | 1.00 | 51.68 | |
| ATOM | 1070 | CD2 | HIS | | 288 | −38.828 | 104.684 | −69.285 | 1.00 | 52.15 | |
| ATOM | 1071 | ND1 | HIS | | 288 | −37.037 | 105.888 | −68.938 | 1.00 | 52.82 | |
| ATOM | 1072 | CE1 | HIS | | 288 | −38.043 | 106.724 | −69.122 | 1.00 | 54.05 | |
| ATOM | 1073 | NE2 | HIS | | 288 | −39.142 | 106.018 | −69.335 | 1.00 | 52.91 | |
| ATOM | 1074 | C | HIS | | 288 | −34.488 | 102.366 | −69.704 | 1.00 | 46.68 | |
| ATOM | 1075 | O | HIS | | 288 | −34.597 | 101.152 | −69.499 | 1.00 | 45.61 | |
| ATOM | 1076 | N | ILE | | 289 | −33.341 | 103.023 | −69.562 | 1.00 | 44.04 | |
| ATOM | 1077 | CA | ILE | | 289 | −32.124 | 102.328 | −69.196 | 1.00 | 43.16 | |
| ATOM | 1078 | CB | ILE | | 289 | −30.914 | 103.271 | −69.247 | 1.00 | 43.33 | |
| ATOM | 1079 | CG2 | ILE | | 289 | −30.311 | 103.442 | −67.857 | 1.00 | 43.11 | |
| ATOM | 1080 | CG1 | ILE | | 289 | −29.907 | 102.722 | −70.263 | 1.00 | 45.10 | |
| ATOM | 1081 | CD1 | ILE | | 289 | −28.589 | 103.483 | −70.385 | 1.00 | 46.75 | |
| ATOM | 1082 | C | ILE | | 289 | −32.140 | 101.607 | −67.851 | 1.00 | 42.02 | |
| ATOM | 1083 | O | ILE | | 289 | −32.802 | 102.022 | −66.893 | 1.00 | 42.42 | |
| ATOM | 1084 | N | LYS | | 290 | −31.416 | 100.498 | −67.807 | 1.00 | 40.39 | |
| ATOM | 1085 | CA | LYS | | 290 | −31.290 | 99.711 | −66.596 | 1.00 | 39.28 | |
| ATOM | 1086 | CB | LYS | | 290 | −32.146 | 98.455 | −66.666 | 1.00 | 38.03 | |
| ATOM | 1087 | CG | LYS | | 290 | −33.543 | 98.601 | −66.124 | 1.00 | 36.87 | |
| ATOM | 1088 | CD | LYS | | 290 | −33.506 | 98.866 | −64.648 | 1.00 | 37.88 | |
| ATOM | 1089 | CE | LYS | | 290 | −34.900 | 99.067 | −64.100 | 1.00 | 38.01 | |
| ATOM | 1090 | NZ | LYS | | 290 | −34.888 | 99.219 | −62.604 | 1.00 | 38.30 | |
| ATOM | 1091 | C | LYS | | 290 | −29.846 | 99.306 | −66.478 | 1.00 | 38.75 | |
| ATOM | 1092 | O | LYS | | 290 | −29.388 | 98.468 | −67.248 | 1.00 | 39.10 | |
| ATOM | 1093 | N | ILE | | 291 | −29.127 | 99.936 | −65.552 | 1.00 | 39.18 | |
| ATOM | 1094 | CA | ILE | | 291 | −27.729 | 99.616 | −65.280 | 1.00 | 40.53 | |
| ATOM | 1095 | CB | ILE | | 291 | −27.102 | 100.659 | −64.371 | 1.00 | 40.48 | |
| ATOM | 1096 | CG2 | ILE | | 291 | −25.636 | 100.353 | −64.175 | 1.00 | 41.38 | |
| ATOM | 1097 | CG1 | ILE | | 291 | −27.296 | 102.050 | −64.980 | 1.00 | 40.46 | |
| ATOM | 1098 | CD1 | ILE | | 291 | −26.629 | 103.149 | −64.222 | 1.00 | 37.02 | |
| ATOM | 1099 | C | ILE | | 291 | −27.739 | 98.275 | −64.546 | 1.00 | 42.36 | |
| ATOM | 1100 | O | ILE | | 291 | −28.198 | 98.160 | −63.417 | 1.00 | 40.43 | |
| ATOM | 1101 | N | THR | | 292 | −27.242 | 97.255 | −65.215 | 1.00 | 46.18 | |
| ATOM | 1102 | CA | THR | | 292 | −27.212 | 95.915 | −64.685 | 1.00 | 50.40 | |
| ATOM | 1103 | CB | THR | | 292 | −27.356 | 94.967 | −65.826 | 1.00 | 50.36 | |
| ATOM | 1104 | OG1 | THR | | 292 | −28.352 | 94.004 | −65.504 | 1.00 | 51.44 | |
| ATOM | 1105 | CG2 | THR | | 292 | −26.031 | 94.303 | −66.124 | 1.00 | 51.92 | |
| ATOM | 1106 | C | THR | | 292 | −25.923 | 95.629 | −63.930 | 1.00 | 53.75 | |
| ATOM | 1107 | O | THR | | 292 | −24.962 | 96.386 | −64.023 | 1.00 | 54.59 | |
| ATOM | 1108 | N | ASP | | 293 | −25.893 | 94.538 | −63.174 | 1.00 | 58.48 | |
| ATOM | 1109 | CA | ASP | | 293 | −24.682 | 94.209 | −62.411 | 1.00 | 62.70 | |
| ATOM | 1110 | CB | ASP | | 293 | −25.012 | 94.041 | −60.917 | 1.00 | 64.65 | |
| ATOM | 1111 | CG | ASP | | 293 | −23.760 | 94.011 | −60.029 | 1.00 | 67.21 | |
| ATOM | 1112 | OD1 | ASP | | 293 | −22.766 | 93.321 | −60.391 | 1.00 | 68.04 | |
| ATOM | 1113 | OD2 | ASP | | 293 | −23.779 | 94.678 | −58.962 | 1.00 | 66.05 | |
| ATOM | 1114 | C | ASP | | 293 | −23.963 | 92.954 | −62.913 | 1.00 | 63.92 | |
| ATOM | 1115 | O | ASP | | 293 | −23.813 | 91.981 | −62.181 | 1.00 | 64.24 | |
| ATOM | 1116 | N | PHE | | 294 | −23.539 | 92.979 | −64.168 | 1.00 | 65.83 | |
| ATOM | 1117 | CA | PHE | | 294 | −22.806 | 91.868 | −64.757 | 1.00 | 68.53 | |
| ATOM | 1118 | CB | PHE | | 294 | −23.521 | 90.530 | −64.491 | 1.00 | 67.38 | |
| ATOM | 1119 | CG | PHE | | 294 | −24.991 | 90.534 | −64.811 | 1.00 | 65.59 | |
| ATOM | 1120 | CD1 | PHE | | 294 | −25.433 | 90.424 | −66.124 | 1.00 | 63.34 | |
| ATOM | 1121 | CD2 | PHE | | 294 | −25.940 | 90.626 | −63.783 | 1.00 | 65.16 | |
| ATOM | 1122 | CE1 | PHE | | 294 | −26.801 | 90.404 | −66.419 | 1.00 | 64.24 | |
| ATOM | 1123 | CE2 | PHE | | 294 | −27.316 | 90.605 | −64.059 | 1.00 | 64.73 | |
| ATOM | 1124 | CZ | PHE | | 294 | −27.750 | 90.494 | −65.385 | 1.00 | 63.90 | |
| ATOM | 1125 | C | PHE | | 294 | −22.551 | 92.046 | −66.254 | 1.00 | 71.35 | |
| ATOM | 1126 | O | PHE | | 294 | −23.113 | 92.943 | −66.899 | 1.00 | 73.04 | |
| ATOM | 1127 | N | GLY | | 295 | −21.679 | 91.196 | −66.793 | 1.00 | 72.68 | |
| ATOM | 1128 | CA | GLY | | 295 | −21.356 | 91.245 | −68.205 | 1.00 | 73.62 | |
| ATOM | 1129 | C | GLY | | 295 | −22.030 | 90.094 | −68.928 | 1.00 | 74.66 | |
| ATOM | 1130 | O | GLY | | 295 | −22.020 | 88.958 | −68.449 | 1.00 | 73.51 | |
| ATOM | 1131 | N | ALA | | 296 | −22.623 | 90.394 | −70.080 | 1.00 | 75.77 | |
| ATOM | 1132 | CA | ALA | | 296 | −23.312 | 89.384 | −70.871 | 1.00 | 76.34 | |
| ATOM | 1133 | CB | ALA | | 296 | −24.361 | 90.050 | −71.752 | 1.00 | 75.21 | |
| ATOM | 1134 | C | ALA | | 296 | −22.313 | 88.596 | −71.733 | 1.00 | 77.70 | |
| ATOM | 1135 | O | ALA | | 296 | −22.126 | 87.378 | −71.560 | 1.00 | 77.57 | |
| ATOM | 1136 | N | THR | | 313 | −14.819 | 92.287 | −75.520 | 1.00 | 61.26 | |
| ATOM | 1137 | CA | THR | | 313 | −14.683 | 93.743 | −75.449 | 1.00 | 61.34 | |
| ATOM | 1138 | CB | THR | | 313 | −16.018 | 94.393 | −74.946 | 1.00 | 62.47 | |
| ATOM | 1139 | OG1 | THR | | 313 | −16.491 | 93.701 | −73.780 | 1.00 | 61.36 | |
| ATOM | 1140 | CG2 | THR | | 313 | −17.105 | 94.324 | −76.048 | 1.00 | 63.50 | |
| ATOM | 1141 | C | THR | | 313 | −13.497 | 94.217 | −74.576 | 1.00 | 59.67 | |
| ATOM | 1142 | O | THR | | 313 | −13.333 | 93.780 | −73.438 | 1.00 | 59.35 | |
| ATOM | 1143 | N | PRO | | 314 | −12.657 | 95.119 | −75.116 | 1.00 | 57.55 | |
| ATOM | 1144 | CD | PRO | | 314 | −12.756 | 95.646 | −76.488 | 1.00 | 56.93 | |
| ATOM | 1145 | CA | PRO | | 314 | −11.480 | 95.677 | −74.429 | 1.00 | 55.87 | |
| ATOM | 1146 | CB | PRO | | 314 | −10.828 | 96.559 | −75.504 | 1.00 | 55.86 | |
| ATOM | 1147 | CG | PRO | | 314 | −11.319 | 95.995 | −76.790 | 1.00 | 57.18 | |
| ATOM | 1148 | C | PRO | | 314 | −11.859 | 96.498 | −73.196 | 1.00 | 54.26 | |
| ATOM | 1149 | O | PRO | | 314 | −12.676 | 97.407 | −73.291 | 1.00 | 55.26 | |
| ATOM | 1150 | N | GLU | | 315 | −11.264 | 96.194 | −72.048 | 1.00 | 52.49 | |
| ATOM | 1151 | CA | GLU | | 315 | −11.561 | 96.934 | −70.828 | 1.00 | 51.35 | |
| ATOM | 1152 | CB | GLU | | 315 | −10.957 | 96.252 | −69.616 | 1.00 | 53.09 | |
| ATOM | 1153 | CG | GLU | | 315 | −11.694 | 95.032 | −69.164 | 1.00 | 60.50 | |
| ATOM | 1154 | CD | GLU | | 315 | −11.734 | 94.927 | −67.643 | 1.00 | 65.03 | |
| ATOM | 1155 | OE1 | GLU | | 315 | −12.688 | 95.482 | −67.023 | 1.00 | 65.47 | |
| ATOM | 1156 | OE2 | GLU | | 315 | −10.797 | 94.304 | −67.073 | 1.00 | 67.26 | |
| ATOM | 1157 | C | GLU | | 315 | −11.030 | 98.355 | −70.865 | 1.00 | 50.05 | |
| ATOM | 1158 | O | GLU | | 315 | −9.906 | 98.591 | −71.284 | 1.00 | 51.67 | |
| ATOM | 1159 | N | TYR | | 316 | −11.834 | 99.314 | −70.428 | 1.00 | 47.11 | |
| ATOM | 1160 | CA | TYR | | 316 | −11.365 | 100.685 | −70.402 | 1.00 | 43.35 | |
| ATOM | 1161 | CB | TYR | | 316 | −12.167 | 101.573 | −71.360 | 1.00 | 40.98 | |
| ATOM | 1162 | CG | TYR | | 316 | −11.678 | 101.609 | −72.786 | 1.00 | 40.11 | |
| ATOM | 1163 | CD1 | TYR | | 316 | −12.024 | 100.610 | −73.685 | 1.00 | 41.02 | |
| ATOM | 1164 | CE1 | TYR | | 316 | −11.627 | 100.660 | −75.021 | 1.00 | 42.06 | |
| ATOM | 1165 | CD2 | TYR | | 316 | −10.903 | 102.679 | −73.255 | 1.00 | 42.01 | |
| ATOM | 1166 | CE2 | TYR | | 316 | −10.489 | 102.754 | −74.609 | 1.00 | 41.47 | |
| ATOM | 1167 | CZ | TYR | | 316 | −10.859 | 101.736 | −75.492 | 1.00 | 44.04 | |
| ATOM | 1168 | OH | TYR | | 316 | −10.487 | 101.776 | −76.844 | 1.00 | 44.56 | |
| ATOM | 1169 | C | TYR | | 316 | −11.474 | 101.212 | −68.974 | 1.00 | 41.97 | |
| ATOM | 1170 | O | TYR | | 316 | −12.354 | 102.030 | −68.648 | 1.00 | 41.74 | |
| ATOM | 1171 | N | LEU | | 317 | −10.601 | 100.709 | −68.113 | 1.00 | 38.94 | |
| ATOM | 1172 | CA | LEU | | 317 | −10.572 | 101.165 | −66.733 | 1.00 | 38.79 | |
| ATOM | 1173 | CB | LEU | | 317 | −9.578 | 100.336 | −65.920 | 1.00 | 37.70 | |
| ATOM | 1174 | CG | LEU | | 317 | −9.799 | 98.842 | −65.770 | 1.00 | 36.96 | |
| ATOM | 1175 | CD1 | LEU | | 317 | −8.726 | 98.351 | −64.804 | 1.00 | 34.85 | |
| ATOM | 1176 | CD2 | LEU | | 317 | −11.207 | 98.517 | −65.258 | 1.00 | 34.60 | |
| ATOM | 1177 | C | LEU | | 317 | −10.123 | 102.633 | −66.655 | 1.00 | 36.86 | |
| ATOM | 1178 | O | LEU | | 317 | −9.390 | 103.107 | −67.515 | 1.00 | 36.87 | |
| ATOM | 1179 | N | ALA | | 318 | −10.540 | 103.346 | −65.618 | 1.00 | 35.70 | |
| ATOM | 1180 | CA | ALA | | 318 | −10.116 | 104.728 | −65.474 | 1.00 | 37.61 | |
| ATOM | 1181 | CB | ALA | | 318 | −10.755 | 105.345 | −64.259 | 1.00 | 37.33 | |
| ATOM | 1182 | C | ALA | | 318 | −8.604 | 104.736 | −65.304 | 1.00 | 38.70 | |
| ATOM | 1183 | O | ALA | | 318 | −8.026 | 103.777 | −64.798 | 1.00 | 40.54 | |
| ATOM | 1184 | N | PRO | | 319 | −7.934 | 105.807 | −65.734 | 1.00 | 38.80 | |
| ATOM | 1185 | CD | PRO | | 319 | −8.351 | 106.906 | −66.606 | 1.00 | 39.29 | |
| ATOM | 1186 | CA | PRO | | 319 | −6.487 | 105.813 | −65.561 | 1.00 | 38.47 | |
| ATOM | 1187 | CB | PRO | | 319 | −6.052 | 107.101 | −66.245 | 1.00 | 37.53 | |
| ATOM | 1188 | CG | PRO | | 319 | −7.287 | 107.900 | −66.314 | 1.00 | 39.33 | |
| ATOM | 1189 | C | PRO | | 319 | −6.046 | 105.739 | −64.114 | 1.00 | 39.51 | |
| ATOM | 1190 | O | PRO | | 319 | −5.009 | 105.149 | −63.820 | 1.00 | 38.97 | |
| ATOM | 1191 | N | GLU | | 320 | −6.820 | 106.309 | −63.198 | 1.00 | 40.43 | |
| ATOM | 1192 | CA | GLU | | 320 | −6.417 | 106.251 | −61.792 | 1.00 | 43.60 | |
| ATOM | 1193 | CB | GLU | | 320 | −7.214 | 107.259 | −60.931 | 1.00 | 44.36 | |
| ATOM | 1194 | CG | GLU | | 320 | −8.720 | 107.052 | −60.916 | 1.00 | 47.85 | |
| ATOM | 1195 | CD | GLU | | 320 | −9.455 | 107.815 | −62.020 | 1.00 | 48.76 | |
| ATOM | 1196 | OE1 | GLU | | 320 | −8.982 | 107.821 | −63.179 | 1.00 | 50.53 | |
| ATOM | 1197 | OE2 | GLU | | 320 | −10.522 | 108.395 | −61.723 | 1.00 | 48.20 | |
| ATOM | 1198 | C | GLU | | 320 | −6.580 | 104.815 | −61.263 | 1.00 | 44.50 | |
| ATOM | 1199 | O | GLU | | 320 | −6.010 | 104.434 | −60.232 | 1.00 | 45.42 | |
| ATOM | 1200 | N | VAL | | 321 | −7.370 | 104.015 | −61.967 | 1.00 | 44.14 | |
| ATOM | 1201 | CA | VAL | | 321 | −7.540 | 102.638 | −61.567 | 1.00 | 45.55 | |
| ATOM | 1202 | CB | VAL | | 321 | −8.818 | 102.032 | −62.206 | 1.00 | 45.38 | |
| ATOM | 1203 | CG1 | VAL | | 321 | −8.820 | 100.525 | −62.069 | 1.00 | 42.63 | |
| ATOM | 1204 | CG2 | VAL | | 321 | −10.038 | 102.607 | −61.530 | 1.00 | 45.16 | |
| ATOM | 1205 | C | VAL | | 321 | −6.271 | 101.898 | −62.041 | 1.00 | 47.14 | |
| ATOM | 1206 | O | VAL | | 321 | −5.714 | 101.074 | −61.308 | 1.00 | 47.99 | |
| ATOM | 1207 | N | LEU | | 322 | −5.812 | 102.222 | −63.254 | 1.00 | 46.59 | |
| ATOM | 1208 | CA | LEU | | 322 | −4.609 | 101.634 | −63.841 | 1.00 | 45.62 | |
| ATOM | 1209 | CB | LEU | | 322 | −4.481 | 102.057 | −65.295 | 1.00 | 42.44 | |
| ATOM | 1210 | CG | LEU | | 322 | −5.494 | 101.455 | −66.250 | 1.00 | 40.66 | |
| ATOM | 1211 | CD1 | LEU | | 322 | −5.222 | 101.899 | −67.698 | 1.00 | 39.43 | |
| ATOM | 1212 | CD2 | LEU | | 322 | −5.410 | 99.965 | −66.116 | 1.00 | 38.54 | |
| ATOM | 1213 | C | LEU | | 322 | −3.312 | 102.023 | −63.131 | 1.00 | 46.94 | |
| ATOM | 1214 | O | LEU | | 322 | −2.427 | 101.191 | −62.905 | 1.00 | 47.93 | |
| ATOM | 1215 | N | GLU | | 323 | −3.188 | 103.290 | −62.782 | 1.00 | 48.12 | |
| ATOM | 1216 | CA | GLU | | 323 | −1.967 | 103.745 | −62.144 | 1.00 | 50.05 | |
| ATOM | 1217 | CB | GLU | | 323 | −1.677 | 105.211 | −62.545 | 1.00 | 48.81 | |
| ATOM | 1218 | CG | GLU | | 323 | −0.359 | 105.785 | −62.024 | 1.00 | 50.87 | |
| ATOM | 1219 | CD | GLU | | 323 | 0.023 | 107.151 | −62.641 | 1.00 | 52.66 | |
| ATOM | 1220 | OE1 | GLU | | 323 | −0.859 | 108.010 | −62.882 | 1.00 | 51.24 | |
| ATOM | 1221 | OE2 | GLU | | 323 | 1.227 | 107.384 | −62.862 | 1.00 | 53.66 | |
| ATOM | 1222 | C | GLU | | 323 | −1.952 | 103.575 | −60.627 | 1.00 | 51.25 | |
| ATOM | 1223 | O | GLU | | 323 | −0.906 | 103.304 | −60.055 | 1.00 | 54.36 | |
| ATOM | 1224 | N | ASP | | 324 | −3.084 | 103.697 | −59.953 | 1.00 | 50.61 | |
| ATOM | 1225 | CA | ASP | | 324 | −3.037 | 103.554 | −58.512 | 1.00 | 50.05 | |
| ATOM | 1226 | CB | ASP | | 324 | −3.044 | 104.925 | −57.846 | 1.00 | 52.30 | |
| ATOM | 1227 | CG | ASP | | 324 | −1.834 | 105.750 | −58.219 | 1.00 | 55.74 | |
| ATOM | 1228 | OD1 | ASP | | 324 | −0.725 | 105.159 | −58.232 | 1.00 | 58.08 | |
| ATOM | 1229 | OD2 | ASP | | 324 | −1.978 | 106.975 | −58.488 | 1.00 | 56.37 | |
| ATOM | 1230 | C | ASP | | 324 | −4.129 | 102.717 | −57.927 | 1.00 | 49.96 | |
| ATOM | 1231 | O | ASP | | 324 | −4.458 | 102.865 | −56.755 | 1.00 | 50.47 | |
| ATOM | 1232 | N | ASN | | 325 | −4.694 | 101.830 | −58.731 | 1.00 | 49.93 | |
| ATOM | 1233 | CA | ASN | | 325 | −5.772 | 100.970 | −58.255 | 1.00 | 49.22 | |
| ATOM | 1234 | CB | ASN | | 325 | −5.184 | 99.796 | −57.495 | 1.00 | 46.61 | |
| ATOM | 1235 | CG | ASN | | 325 | −6.211 | 98.775 | −57.158 | 1.00 | 47.05 | |
| ATOM | 1236 | OD1 | ASN | | 325 | −6.958 | 98.324 | −58.033 | 1.00 | 47.61 | |
| ATOM | 1237 | ND2 | ASN | | 325 | −6.269 | 98.389 | −55.888 | 1.00 | 44.60 | |
| ATOM | 1238 | C | ASN | | 325 | −6.754 | 101.742 | −57.352 | 1.00 | 49.53 | |
| ATOM | 1239 | O | ASN | | 325 | −7.311 | 101.204 | −56.393 | 1.00 | 49.05 | |
| ATOM | 1240 | N | ASP | | 326 | −6.962 | 103.008 | −57.704 | 1.00 | 50.17 | |
| ATOM | 1241 | CA | ASP | | 326 | −7.831 | 103.937 | −56.975 | 1.00 | 50.18 | |
| ATOM | 1242 | CB | ASP | | 326 | −7.149 | 105.309 | −56.995 | 1.00 | 53.10 | |
| ATOM | 1243 | CG | ASP | | 326 | −7.929 | 106.362 | −56.273 | 1.00 | 56.35 | |
| ATOM | 1244 | OD1 | ASP | | 326 | −7.509 | 107.537 | −56.325 | 1.00 | 58.45 | |
| ATOM | 1245 | OD2 | ASP | | 326 | −8.955 | 106.024 | −55.655 | 1.00 | 58.70 | |
| ATOM | 1246 | C | ASP | | 326 | −9.273 | 104.030 | −57.540 | 1.00 | 48.15 | |
| ATOM | 1247 | O | ASP | | 326 | −9.510 | 104.626 | −58.591 | 1.00 | 47.03 | |
| ATOM | 1248 | N | TYR | | 327 | −10.228 | 103.433 | −56.836 | 1.00 | 45.99 | |
| ATOM | 1249 | CA | TYR | | 327 | −11.616 | 103.447 | −57.281 | 1.00 | 45.38 | |
| ATOM | 1250 | CB | TYR | | 327 | −12.271 | 102.063 | −57.137 | 1.00 | 43.12 | |
| ATOM | 1251 | CG | TYR | | 327 | −11.819 | 101.000 | −58.118 | 1.00 | 42.93 | |
| ATOM | 1252 | CD1 | TYR | | 327 | −10.610 | 100.306 | −57.946 | 1.00 | 42.61 | |
| ATOM | 1253 | CE1 | TYR | | 327 | −10.225 | 99.290 | −58.835 | 1.00 | 42.42 | |
| ATOM | 1254 | CD2 | TYR | | 327 | −12.614 | 100.658 | −59.203 | 1.00 | 42.05 | |
| ATOM | 1255 | CE2 | TYR | | 327 | −12.238 | 99.657 | −60.100 | 1.00 | 41.56 | |
| ATOM | 1256 | CZ | TYR | | 327 | −11.048 | 98.970 | −59.919 | 1.00 | 42.45 | |
| ATOM | 1257 | OH | TYR | | 327 | −10.694 | 97.968 | −60.826 | 1.00 | 41.82 | |
| ATOM | 1258 | C | TYR | | 327 | −12.465 | 104.444 | −56.496 | 1.00 | 46.49 | |
| ATOM | 1259 | O | TYR | | 327 | −13.230 | 104.042 | −55.614 | 1.00 | 46.90 | |
| ATOM | 1260 | N | GLY | | 328 | −12.333 | 105.733 | −56.807 | 1.00 | 45.77 | |
| ATOM | 1261 | CA | GLY | | 328 | −13.149 | 106.738 | −56.142 | 1.00 | 45.22 | |
| ATOM | 1262 | C | GLY | | 328 | −14.339 | 107.091 | −57.033 | 1.00 | 45.48 | |
| ATOM | 1263 | O | GLY | | 328 | −14.499 | 106.496 | −58.114 | 1.00 | 45.70 | |
| ATOM | 1264 | N | ARG | | 329 | −15.173 | 108.037 | −56.591 | 1.00 | 43.69 | |
| ATOM | 1265 | CA | ARG | | 329 | −16.339 | 108.486 | −57.361 | 1.00 | 42.80 | |
| ATOM | 1266 | CB | ARG | | 329 | −16.799 | 109.864 | −56.888 | 1.00 | 44.61 | |
| ATOM | 1267 | CG | ARG | | 329 | −17.258 | 110.041 | −55.457 | 1.00 | 44.55 | |
| ATOM | 1268 | CD | ARG | | 329 | −16.877 | 111.454 | −55.025 | 1.00 | 43.18 | |
| ATOM | 1269 | NE | ARG | | 329 | −16.989 | 112.379 | −56.143 | 1.00 | 41.78 | |
| ATOM | 1270 | CZ | ARG | | 329 | −16.297 | 113.511 | −56.280 | 1.00 | 43.13 | |
| ATOM | 1271 | NH1 | ARG | | 329 | −15.409 | 113.903 | −55.368 | 1.00 | 40.29 | |
| ATOM | 1272 | NH2 | ARG | | 329 | −16.497 | 114.263 | −57.355 | 1.00 | 44.31 | |
| ATOM | 1273 | C | ARG | | 329 | −16.014 | 108.652 | −58.845 | 1.00 | 42.35 | |
| ATOM | 1274 | O | ARG | | 329 | −16.679 | 108.105 | −59.713 | 1.00 | 42.55 | |
| ATOM | 1275 | N | ALA | | 330 | −14.996 | 109.464 | −59.109 | 1.00 | 41.66 | |
| ATOM | 1276 | CA | ALA | | 330 | −14.552 | 109.782 | −60.453 | 1.00 | 41.23 | |
| ATOM | 1277 | CB | ALA | | 330 | −13.212 | 110.456 | −60.386 | 1.00 | 40.72 | |
| ATOM | 1278 | C | ALA | | 330 | −14.513 | 108.637 | −61.471 | 1.00 | 41.72 | |
| ATOM | 1279 | O | ALA | | 330 | −14.745 | 108.868 | −62.649 | 1.00 | 43.52 | |
| ATOM | 1280 | N | VAL | | 331 | −14.235 | 107.408 | −61.049 | 1.00 | 41.60 | |
| ATOM | 1281 | CA | VAL | | 331 | −14.193 | 106.309 | −62.017 | 1.00 | 40.30 | |
| ATOM | 1282 | CB | VAL | | 331 | −13.656 | 104.980 | −61.391 | 1.00 | 38.70 | |
| ATOM | 1283 | CG1 | VAL | | 331 | −12.310 | 105.224 | −60.787 | 1.00 | 38.40 | |
| ATOM | 1284 | CG2 | VAL | | 331 | −14.604 | 104.436 | −60.355 | 1.00 | 38.50 | |
| ATOM | 1285 | C | VAL | | 331 | −15.552 | 106.049 | −62.654 | 1.00 | 39.89 | |
| ATOM | 1286 | O | VAL | | 331 | −15.632 | 105.459 | −63.733 | 1.00 | 40.50 | |
| ATOM | 1287 | N | ASP | | 332 | −16.615 | 106.487 | −61.984 | 1.00 | 38.53 | |
| ATOM | 1288 | CA | ASP | | 332 | −17.958 | 106.304 | −62.515 | 1.00 | 39.28 | |
| ATOM | 1289 | CB | ASP | | 332 | −19.007 | 106.559 | −61.441 | 1.00 | 40.53 | |
| ATOM | 1290 | CG | ASP | | 332 | −19.059 | 105.468 | −60.439 | 1.00 | 42.03 | |
| ATOM | 1291 | OD1 | ASP | | 332 | −18.868 | 104.312 | −60.876 | 1.00 | 44.04 | |
| ATOM | 1292 | OD2 | ASP | | 332 | −19.294 | 105.758 | −59.241 | 1.00 | 42.18 | |
| ATOM | 1293 | C | ASP | | 332 | −18.222 | 107.243 | −63.679 | 1.00 | 38.44 | |
| ATOM | 1294 | O | ASP | | 332 | −18.936 | 106.901 | −64.630 | 1.00 | 36.56 | |
| ATOM | 1295 | N | TRP | | 333 | −17.630 | 108.427 | −63.595 | 1.00 | 37.75 | |
| ATOM | 1296 | CA | TRP | | 333 | −17.811 | 109.449 | −64.607 | 1.00 | 37.81 | |
| ATOM | 1297 | CB | TRP | | 333 | −17.399 | 110.803 | −64.025 | 1.00 | 36.84 | |
| ATOM | 1298 | CG | TRP | | 333 | −18.360 | 111.209 | −62.930 | 1.00 | 35.29 | |
| ATOM | 1299 | CD2 | TRP | | 333 | −19.794 | 111.187 | −63.007 | 1.00 | 32.65 | |
| ATOM | 1300 | CE2 | TRP | | 333 | −20.284 | 111.584 | −61.744 | 1.00 | 32.66 | |
| ATOM | 1301 | CE3 | TRP | | 333 | −20.704 | 110.885 | −64.023 | 1.00 | 32.50 | |
| ATOM | 1302 | CD1 | TRP | | 333 | −18.048 | 111.605 | −61.662 | 1.00 | 33.63 | |
| ATOM | 1303 | NE1 | TRP | | 333 | −19.202 | 111.826 | −60.941 | 1.00 | 33.21 | |
| ATOM | 1304 | CZ2 | TRP | | 333 | −21.659 | 111.682 | −61.467 | 1.00 | 32.05 | |
| ATOM | 1305 | CZ3 | TRP | | 333 | −22.075 | 110.984 | −63.749 | 1.00 | 33.26 | |
| ATOM | 1306 | CH2 | TRP | | 333 | −22.536 | 111.385 | −62.476 | 1.00 | 30.12 | |
| ATOM | 1307 | C | TRP | | 333 | −17.035 | 109.095 | −65.849 | 1.00 | 39.56 | |
| ATOM | 1308 | O | TRP | | 333 | −17.500 | 109.328 | −66.973 | 1.00 | 40.91 | |
| ATOM | 1309 | N | TRP | | 334 | −15.864 | 108.504 | −65.637 | 1.00 | 39.04 | |
| ATOM | 1310 | CA | TRP | | 334 | −15.028 | 108.051 | −66.720 | 1.00 | 37.22 | |
| ATOM | 1311 | CB | TRP | | 334 | −13.735 | 107.505 | −66.121 | 1.00 | 40.96 | |
| ATOM | 1312 | CG | TRP | | 334 | −12.918 | 106.669 | −67.041 | 1.00 | 42.79 | |
| ATOM | 1313 | CD2 | TRP | | 334 | −11.938 | 107.140 | −67.955 | 1.00 | 43.52 | |
| ATOM | 1314 | CE2 | TRP | | 334 | −11.464 | 106.020 | −68.673 | 1.00 | 43.53 | |
| ATOM | 1315 | CE3 | TRP | | 334 | −11.420 | 108.406 | −68.251 | 1.00 | 44.18 | |
| ATOM | 1316 | CD1 | TRP | | 334 | −12.995 | 105.317 | −67.217 | 1.00 | 41.70 | |
| ATOM | 1317 | NE1 | TRP | | 334 | −12.124 | 104.918 | −68.198 | 1.00 | 42.64 | |
| ATOM | 1318 | CZ2 | TRP | | 334 | −10.488 | 106.127 | −69.667 | 1.00 | 43.97 | |
| ATOM | 1319 | CZ3 | TRP | | 334 | −10.454 | 108.514 | −69.236 | 1.00 | 43.63 | |
| ATOM | 1320 | CH2 | TRP | | 334 | −10.000 | 107.379 | −69.938 | 1.00 | 42.90 | |
| ATOM | 1321 | C | TRP | | 334 | −15.841 | 106.971 | −67.442 | 1.00 | 35.47 | |
| ATOM | 1322 | O | TRP | | 334 | −15.970 | 106.961 | −68.661 | 1.00 | 36.91 | |
| ATOM | 1323 | N | GLY | | 335 | −16.425 | 106.072 | −66.674 | 1.00 | 34.13 | |
| ATOM | 1324 | CA | GLY | | 335 | −17.221 | 105.030 | −67.277 | 1.00 | 33.20 | |
| ATOM | 1325 | C | GLY | | 335 | −18.317 | 105.669 | −68.081 | 1.00 | 34.33 | |
| ATOM | 1326 | O | GLY | | 335 | −18.660 | 105.208 | −69.176 | 1.00 | 34.34 | |
| ATOM | 1327 | N | LEU | | 336 | −18.872 | 106.745 | −67.535 | 1.00 | 33.92 | |
| ATOM | 1328 | CA | LEU | | 336 | −19.943 | 107.457 | −68.211 | 1.00 | 33.63 | |
| ATOM | 1329 | CB | LEU | | 336 | −20.366 | 108.695 | −67.430 | 1.00 | 33.05 | |
| ATOM | 1330 | CG | LEU | | 336 | −21.493 | 109.476 | −68.121 | 1.00 | 30.46 | |
| ATOM | 1331 | CD1 | LEU | | 336 | −22.685 | 108.555 | −68.460 | 1.00 | 31.04 | |
| ATOM | 1332 | CD2 | LEU | | 336 | −21.898 | 110.613 | −67.216 | 1.00 | 28.30 | |
| ATOM | 1333 | C | LEU | | 336 | −19.469 | 107.897 | −69.565 | 1.00 | 33.40 | |
| ATOM | 1334 | O | LEU | | 336 | −20.042 | 107.516 | −70.592 | 1.00 | 33.66 | |
| ATOM | 1335 | N | GLY | | 337 | −18.420 | 108.713 | −69.537 | 1.00 | 32.38 | |
| ATOM | 1336 | CA | GLY | | 337 | −17.821 | 109.237 | −70.742 | 1.00 | 32.93 | |
| ATOM | 1337 | C | GLY | | 337 | −17.460 | 108.178 | −71.762 | 1.00 | 34.46 | |
| ATOM | 1338 | O | GLY | | 337 | −17.879 | 108.281 | −72.924 | 1.00 | 36.54 | |
| ATOM | 1339 | N | VAL | | 338 | −16.670 | 107.182 | −71.374 | 1.00 | 32.43 | |
| ATOM | 1340 | CA | VAL | | 338 | −16.345 | 106.136 | −72.341 | 1.00 | 33.15 | |
| ATOM | 1341 | CB | VAL | | 338 | −15.505 | 104.962 | −71.707 | 1.00 | 32.41 | |
| ATOM | 1342 | CG1 | VAL | | 338 | −15.446 | 103.780 | −72.680 | 1.00 | 27.57 | |
| ATOM | 1343 | CG2 | VAL | | 338 | −14.083 | 105.438 | −71.373 | 1.00 | 30.48 | |
| ATOM | 1344 | C | VAL | | 338 | −17.636 | 105.557 | −72.944 | 1.00 | 32.28 | |
| ATOM | 1345 | O | VAL | | 338 | −17.672 | 105.215 | −74.106 | 1.00 | 31.83 | |
| ATOM | 1346 | N | VAL | | 339 | −18.694 | 105.455 | −72.158 | 1.00 | 33.11 | |
| ATOM | 1347 | CA | VAL | | 339 | −19.926 | 104.898 | −72.694 | 1.00 | 37.56 | |
| ATOM | 1348 | CB | VAL | | 339 | −20.964 | 104.598 | −71.598 | 1.00 | 36.78 | |
| ATOM | 1349 | CG1 | VAL | | 339 | −22.313 | 104.291 | −72.226 | 1.00 | 34.38 | |
| ATOM | 1350 | CG2 | VAL | | 339 | −20.506 | 103.436 | −70.769 | 1.00 | 34.72 | |
| ATOM | 1351 | C | VAL | | 339 | −20.548 | 105.866 | −73.674 | 1.00 | 41.60 | |
| ATOM | 1352 | O | VAL | | 339 | −20.890 | 105.509 | −74.794 | 1.00 | 43.18 | |
| ATOM | 1353 | N | MET | | 340 | −20.710 | 107.105 | −73.272 | 1.00 | 44.34 | |
| ATOM | 1354 | CA | MET | | 340 | −21.305 | 107.998 | −74.217 | 1.00 | 47.85 | |
| ATOM | 1355 | CB | MET | | 340 | −21.634 | 109.312 | −73.541 | 1.00 | 48.32 | |
| ATOM | 1356 | CG | MET | | 340 | −22.880 | 109.147 | −72.735 | 1.00 | 51.33 | |
| ATOM | 1357 | SD | MET | | 340 | −23.443 | 110.658 | −72.056 | 1.00 | 59.88 | |
| ATOM | 1358 | CE | MET | | 340 | −22.191 | 110.901 | −70.873 | 1.00 | 58.75 | |
| ATOM | 1359 | C | MET | | 340 | −20.435 | 108.160 | −75.464 | 1.00 | 50.14 | |
| ATOM | 1360 | O | MET | | 340 | −20.967 | 108.305 | −76.579 | 1.00 | 52.11 | |
| ATOM | 1361 | N | TYR | | 341 | −19.114 | 108.100 | −75.301 | 1.00 | 49.46 | |
| ATOM | 1362 | CA | TYR | | 341 | −18.229 | 108.223 | −76.455 | 1.00 | 48.67 | |
| ATOM | 1363 | CB | TYR | | 341 | −16.766 | 108.132 | −76.045 | 1.00 | 48.69 | |
| ATOM | 1364 | CG | TYR | | 341 | −15.774 | 108.344 | −77.181 | 1.00 | 47.70 | |
| ATOM | 1365 | CD1 | TYR | | 341 | −15.734 | 107.493 | −78.299 | 1.00 | 46.57 | |
| ATOM | 1366 | CE1 | TYR | | 341 | −14.791 | 107.668 | −79.308 | 1.00 | 45.67 | |
| ATOM | 1367 | CD2 | TYR | | 341 | −14.847 | 109.377 | −77.110 | 1.00 | 47.95 | |
| ATOM | 1368 | CE2 | TYR | | 341 | −13.900 | 109.564 | −78.105 | 1.00 | 48.62 | |
| ATOM | 1369 | CZ | TYR | | 341 | −13.869 | 108.715 | −79.202 | 1.00 | 48.25 | |
| ATOM | 1370 | OH | TYR | | 341 | −12.900 | 108.947 | −80.164 | 1.00 | 46.56 | |
| ATOM | 1371 | C | TYR | | 341 | −18.499 | 107.126 | −77.467 | 1.00 | 48.36 | |
| ATOM | 1372 | O | TYR | | 341 | −18.438 | 107.354 | −78.669 | 1.00 | 48.58 | |
| ATOM | 1373 | N | GLU | | 342 | −18.793 | 105.926 | −76.998 | 1.00 | 47.95 | |
| ATOM | 1374 | CA | GLU | | 342 | −19.029 | 104.874 | −77.949 | 1.00 | 48.49 | |
| ATOM | 1375 | CB | GLU | | 342 | −19.093 | 103.512 | −77.274 | 1.00 | 50.95 | |
| ATOM | 1376 | CG | GLU | | 342 | −18.572 | 102.395 | −78.172 | 1.00 | 54.82 | |
| ATOM | 1377 | CD | GLU | | 342 | −18.836 | 101.005 | −77.608 | 1.00 | 57.85 | |
| ATOM | 1378 | OE1 | GLU | | 342 | −18.663 | 100.818 | −76.382 | 1.00 | 59.56 | |
| ATOM | 1379 | OE2 | GLU | | 342 | −19.206 | 100.094 | −78.390 | 1.00 | 57.97 | |
| ATOM | 1380 | C | GLU | | 342 | −20.332 | 105.178 | −78.612 | 1.00 | 48.20 | |
| ATOM | 1381 | O | GLU | | 342 | −20.476 | 105.012 | −79.812 | 1.00 | 48.67 | |
| ATOM | 1382 | N | MET | | 343 | −21.275 | 105.673 | −77.826 | 1.00 | 49.06 | |
| ATOM | 1383 | CA | MET | | 343 | −22.607 | 105.984 | −78.336 | 1.00 | 48.83 | |
| ATOM | 1384 | CB | MET | | 343 | −23.518 | 106.434 | −77.192 | 1.00 | 48.54 | |
| ATOM | 1385 | CG | MET | | 343 | −24.128 | 105.302 | −76.390 | 1.00 | 47.29 | |
| ATOM | 1386 | SD | MET | | 343 | −25.065 | 105.970 | −75.026 | 1.00 | 47.95 | |
| ATOM | 1387 | CE | MET | | 343 | −26.127 | 104.620 | −74.632 | 1.00 | 45.95 | |
| ATOM | 1388 | C | MET | | 343 | −22.691 | 107.006 | −79.467 | 1.00 | 49.03 | |
| ATOM | 1389 | O | MET | | 343 | −23.331 | 106.772 | −80.494 | 1.00 | 47.16 | |
| ATOM | 1390 | N | MET | | 344 | −22.053 | 108.149 | −79.274 | 1.00 | 49.81 | |
| ATOM | 1391 | CA | MET | | 344 | −22.091 | 109.194 | −80.277 | 1.00 | 50.59 | |
| ATOM | 1392 | CB | MET | | 344 | −21.991 | 110.552 | −79.571 | 1.00 | 50.34 | |
| ATOM | 1393 | CG | MET | | 344 | −23.211 | 110.906 | −78.688 | 1.00 | 49.99 | |
| ATOM | 1394 | SD | MET | | 344 | −22.884 | 112.235 | −77.445 | 1.00 | 50.65 | |
| ATOM | 1395 | CE | MET | | 344 | −21.609 | 113.102 | −78.152 | 1.00 | 51.66 | |
| ATOM | 1396 | C | MET | | 344 | −20.993 | 109.033 | −81.344 | 1.00 | 52.24 | |
| ATOM | 1397 | O | MET | | 344 | −20.998 | 109.744 | −82.353 | 1.00 | 52.10 | |
| ATOM | 1398 | N | CYS | | 345 | −20.066 | 108.090 | −81.133 | 1.00 | 52.55 | |
| ATOM | 1399 | CA | CYS | | 345 | −18.983 | 107.863 | −82.086 | 1.00 | 51.46 | |
| ATOM | 1400 | CB | CYS | | 345 | −17.639 | 108.112 | −81.425 | 1.00 | 52.21 | |
| ATOM | 1401 | SG | CYS | | 345 | −17.325 | 109.829 | −80.872 | 1.00 | 52.31 | |
| ATOM | 1402 | C | CYS | | 345 | −18.984 | 106.472 | −82.725 | 1.00 | 53.00 | |
| ATOM | 1403 | O | CYS | | 345 | −18.323 | 106.263 | −83.747 | 1.00 | 53.87 | |
| ATOM | 1404 | N | GLY | | 346 | −19.724 | 105.528 | −82.132 | 1.00 | 53.88 | |
| ATOM | 1405 | CA | GLY | | 346 | −19.820 | 104.162 | −82.660 | 1.00 | 54.16 | |
| ATOM | 1406 | C | GLY | | 346 | −18.594 | 103.258 | −82.487 | 1.00 | 55.22 | |
| ATOM | 1407 | O | GLY | | 346 | −18.526 | 102.127 | −82.996 | 1.00 | 54.37 | |
| ATOM | 1408 | N | ARG | | 347 | −17.622 | 103.735 | −81.730 | 1.00 | 55.38 | |
| ATOM | 1409 | CA | ARG | | 347 | −16.408 | 102.972 | −81.559 | 1.00 | 56.01 | |
| ATOM | 1410 | CB | ARG | | 347 | −15.562 | 103.138 | −82.817 | 1.00 | 57.25 | |
| ATOM | 1411 | CG | ARG | | 347 | −15.291 | 104.588 | −83.115 | 1.00 | 59.86 | |
| ATOM | 1412 | CD | ARG | | 347 | −14.333 | 104.760 | −84.255 | 1.00 | 64.85 | |
| ATOM | 1413 | NE | ARG | | 347 | −13.725 | 106.094 | −84.271 | 1.00 | 70.18 | |
| ATOM | 1414 | CZ | ARG | | 347 | −14.316 | 107.196 | −84.741 | 1.00 | 73.20 | |
| ATOM | 1415 | NH1 | ARG | | 347 | −15.553 | 107.134 | −85.244 | 1.00 | 73.23 | |
| ATOM | 1416 | NH2 | ARG | | 347 | −13.662 | 108.365 | −84.720 | 1.00 | 74.67 | |
| ATOM | 1417 | C | ARG | | 347 | −15.674 | 103.518 | −80.345 | 1.00 | 54.82 | |
| ATOM | 1418 | O | ARG | | 347 | −15.829 | 104.704 | −80.000 | 1.00 | 54.57 | |
| ATOM | 1419 | N | LEU | | 348 | −14.873 | 102.664 | −79.705 | 1.00 | 52.56 | |
| ATOM | 1420 | CA | LEU | | 348 | −14.136 | 103.085 | −78.515 | 1.00 | 50.40 | |
| ATOM | 1421 | CB | LEU | | 348 | −13.570 | 101.869 | −77.791 | 1.00 | 47.48 | |
| ATOM | 1422 | CG | LEU | | 348 | −14.693 | 100.992 | −77.231 | 1.00 | 45.56 | |
| ATOM | 1423 | CD1 | LEU | | 348 | −14.148 | 99.664 | −76.836 | 1.00 | 44.34 | |
| ATOM | 1424 | CD2 | LEU | | 348 | −15.354 | 101.671 | −76.041 | 1.00 | 45.21 | |
| ATOM | 1425 | C | LEU | | 348 | −13.043 | 104.076 | −78.866 | 1.00 | 50.04 | |
| ATOM | 1426 | O | LEU | | 348 | −12.529 | 104.088 | −79.976 | 1.00 | 48.92 | |
| ATOM | 1427 | N | PRO | | 349 | −12.683 | 104.939 | −77.917 | 1.00 | 51.10 | |
| ATOM | 1428 | CD | PRO | | 349 | −13.202 | 105.059 | −76.548 | 1.00 | 50.72 | |
| ATOM | 1429 | CA | PRO | | 349 | −11.642 | 105.935 | −78.185 | 1.00 | 52.64 | |
| ATOM | 1430 | CB | PRO | | 349 | −11.346 | 106.542 | −76.798 | 1.00 | 51.49 | |
| ATOM | 1431 | CG | PRO | | 349 | −12.012 | 105.631 | −75.823 | 1.00 | 51.83 | |
| ATOM | 1432 | C | PRO | | 349 | −10.400 | 105.444 | −78.917 | 1.00 | 53.85 | |
| ATOM | 1433 | O | PRO | | 349 | −9.817 | 106.185 | −79.706 | 1.00 | 53.86 | |
| ATOM | 1434 | N | PHE | | 350 | −10.006 | 104.201 | −78.686 | 1.00 | 56.07 | |
| ATOM | 1435 | CA | PHE | | 350 | −8.825 | 103.663 | −79.350 | 1.00 | 59.34 | |
| ATOM | 1436 | CB | PHE | | 350 | −7.741 | 103.367 | −78.303 | 1.00 | 55.77 | |
| ATOM | 1437 | CG | PHE | | 350 | −7.378 | 104.546 | −77.417 | 1.00 | 52.76 | |
| ATOM | 1438 | CD1 | PHE | | 350 | −7.568 | 104.479 | −76.033 | 1.00 | 51.68 | |
| ATOM | 1439 | CD2 | PHE | | 350 | −6.749 | 105.677 | −77.944 | 1.00 | 48.51 | |
| ATOM | 1440 | CE1 | PHE | | 350 | −7.124 | 105.513 | −75.194 | 1.00 | 48.61 | |
| ATOM | 1441 | CE2 | PHE | | 350 | −6.309 | 106.703 | −77.116 | 1.00 | 46.48 | |
| ATOM | 1442 | CZ | PHE | | 350 | −6.492 | 106.622 | −75.739 | 1.00 | 47.76 | |
| ATOM | 1443 | C | PHE | | 350 | −9.178 | 102.367 | −80.105 | 1.00 | 63.66 | |
| ATOM | 1444 | O | PHE | | 350 | −8.379 | 101.437 | −80.170 | 1.00 | 65.35 | |
| ATOM | 1445 | N | TYR | | 351 | −10.367 | 102.318 | −80.691 | 1.00 | 68.61 | |
| ATOM | 1446 | CA | TYR | | 351 | −10.858 | 101.121 | −81.386 | 1.00 | 73.08 | |
| ATOM | 1447 | CB | TYR | | 351 | −12.190 | 101.415 | −82.076 | 1.00 | 74.99 | |
| ATOM | 1448 | CG | TYR | | 351 | −12.014 | 102.042 | −83.441 | 1.00 | 77.23 | |
| ATOM | 1449 | CD1 | TYR | | 351 | −12.362 | 101.340 | −84.604 | 1.00 | 78.33 | |
| ATOM | 1450 | CE1 | TYR | | 351 | −12.205 | 101.917 | −85.869 | 1.00 | 79.59 | |
| ATOM | 1451 | CD2 | TYR | | 351 | −11.497 | 103.334 | −83.574 | 1.00 | 77.34 | |
| ATOM | 1452 | CE2 | TYR | | 351 | −11.337 | 103.920 | −84.829 | 1.00 | 79.10 | |
| ATOM | 1453 | CZ | TYR | | 351 | −11.697 | 103.211 | −85.970 | 1.00 | 80.28 | |
| ATOM | 1454 | OH | TYR | | 351 | −11.601 | 103.815 | −87.202 | 1.00 | 82.10 | |
| ATOM | 1455 | C | TYR | | 351 | −9.962 | 100.449 | −82.414 | 1.00 | 75.11 | |
| ATOM | 1456 | O | TYR | | 351 | −10.107 | 99.246 | −82.654 | 1.00 | 76.83 | |
| ATOM | 1457 | N | ASN | | 352 | −9.067 | 101.196 | −83.050 | 1.00 | 75.74 | |
| ATOM | 1458 | CA | ASN | | 352 | −8.216 | 100.575 | −84.056 | 1.00 | 77.97 | |
| ATOM | 1459 | CB | ASN | | 352 | −8.127 | 101.479 | −85.276 | 1.00 | 79.00 | |
| ATOM | 1460 | CG | ASN | | 352 | −7.847 | 102.900 | −84.909 | 1.00 | 80.39 | |
| ATOM | 1461 | OD1 | ASN | | 352 | −8.034 | 103.806 | −85.714 | 1.00 | 80.82 | |
| ATOM | 1462 | ND2 | ASN | | 352 | −7.390 | 103.112 | −83.683 | 1.00 | 81.68 | |
| ATOM | 1463 | C | ASN | | 352 | −6.819 | 100.177 | −83.574 | 1.00 | 79.11 | |
| ATOM | 1464 | O | ASN | | 352 | −6.396 | 99.041 | −83.792 | 1.00 | 79.92 | |
| ATOM | 1465 | N | GLN | | 353 | −6.110 | 101.090 | −82.917 | 1.00 | 79.23 | |
| ATOM | 1466 | CA | GLN | | 353 | −4.773 | 100.797 | −82.408 | 1.00 | 79.53 | |
| ATOM | 1467 | CB | GLN | | 353 | −4.298 | 101.949 | −81.519 | 1.00 | 78.93 | |
| ATOM | 1468 | CG | GLN | | 353 | −4.329 | 103.304 | −82.207 | 1.00 | 78.41 | |
| ATOM | 1469 | CD | GLN | | 353 | −3.652 | 104.395 | −81.391 | 1.00 | 79.43 | |
| ATOM | 1470 | OE1 | GLN | | 353 | −2.433 | 104.374 | −81.175 | 1.00 | 79.09 | |
| ATOM | 1471 | NE2 | GLN | | 353 | −4.444 | 105.359 | −80.931 | 1.00 | 79.05 | |
| ATOM | 1472 | C | GLN | | 353 | −4.706 | 99.462 | −81.640 | 1.00 | 80.21 | |
| ATOM | 1473 | O | GLN | | 353 | −5.408 | 99.257 | −80.652 | 1.00 | 80.41 | |
| ATOM | 1474 | N | ASP | | 354 | −3.836 | 98.573 | −82.113 | 1.00 | 81.30 | |
| ATOM | 1475 | CA | ASP | | 354 | −3.630 | 97.229 | −81.561 | 1.00 | 82.19 | |
| ATOM | 1476 | CB | ASP | | 354 | −2.186 | 97.069 | −81.045 | 1.00 | 82.85 | |
| ATOM | 1477 | CG | ASP | | 354 | −1.707 | 95.603 | −81.059 | 1.00 | 83.70 | |
| ATOM | 1478 | OD1 | ASP | | 354 | −2.412 | 94.722 | −80.500 | 1.00 | 84.53 | |
| ATOM | 1479 | OD2 | ASP | | 354 | −0.620 | 95.337 | −81.624 | 1.00 | 82.33 | |
| ATOM | 1480 | C | ASP | | 354 | −4.618 | 96.764 | −80.483 | 1.00 | 81.95 | |
| ATOM | 1481 | O | ASP | | 354 | −5.694 | 96.258 | −80.813 | 1.00 | 82.11 | |
| ATOM | 1482 | N | HIS | | 355 | −4.250 | 96.924 | −79.210 | 1.00 | 81.03 | |
| ATOM | 1483 | CA | HIS | | 355 | −5.094 | 96.489 | −78.096 | 1.00 | 80.71 | |
| ATOM | 1484 | CB | HIS | | 355 | −5.745 | 95.142 | −78.415 | 1.00 | 82.86 | |
| ATOM | 1485 | CG | HIS | | 355 | −6.471 | 94.531 | −77.254 | 1.00 | 85.76 | |
| ATOM | 1486 | CD2 | HIS | | 355 | −6.160 | 93.475 | −76.467 | 1.00 | 87.26 | |
| ATOM | 1487 | ND1 | HIS | | 355 | −7.660 | 95.032 | −76.769 | 1.00 | 87.41 | |
| ATOM | 1488 | CE1 | HIS | | 355 | −8.050 | 94.311 | −75.733 | 1.00 | 87.85 | |
| ATOM | 1489 | NE2 | HIS | | 355 | −7.159 | 93.359 | −75.527 | 1.00 | 87.74 | |
| ATOM | 1490 | C | HIS | | 355 | −4.260 | 96.338 | −76.833 | 1.00 | 79.81 | |
| ATOM | 1491 | O | HIS | | 355 | −4.703 | 96.661 | −75.730 | 1.00 | 79.19 | |
| ATOM | 1492 | N | GLU | | 356 | −3.055 | 95.810 | −77.005 | 1.00 | 79.13 | |
| ATOM | 1493 | CA | GLU | | 356 | −2.133 | 95.617 | −75.896 | 1.00 | 77.59 | |
| ATOM | 1494 | CB | GLU | | 356 | −1.061 | 94.615 | −76.289 | 1.00 | 80.02 | |
| ATOM | 1495 | CG | GLU | | 356 | −1.624 | 93.352 | −76.907 | 1.00 | 83.48 | |
| ATOM | 1496 | CD | GLU | | 356 | −0.573 | 92.577 | −77.683 | 1.00 | 86.23 | |
| ATOM | 1497 | OE1 | GLU | | 356 | −0.023 | 93.137 | −78.661 | 1.00 | 86.65 | |
| ATOM | 1498 | OE2 | GLU | | 356 | −0.296 | 91.412 | −77.315 | 1.00 | 87.72 | |
| ATOM | 1499 | C | GLU | | 356 | −1.511 | 96.980 | −75.633 | 1.00 | 75.49 | |
| ATOM | 1500 | O | GLU | | 356 | −0.698 | 97.156 | −74.723 | 1.00 | 74.40 | |
| ATOM | 1501 | N | ARG | | 357 | −1.916 | 97.937 | −76.461 | 1.00 | 72.73 | |
| ATOM | 1502 | CA | ARG | | 357 | −1.466 | 99.306 | −76.361 | 1.00 | 70.48 | |
| ATOM | 1503 | CB | ARG | | 357 | −1.546 | 99.964 | −77.735 | 1.00 | 72.58 | |
| ATOM | 1504 | CG | ARG | | 357 | −0.207 | 100.160 | −78.398 | 1.00 | 77.63 | |
| ATOM | 1505 | CD | ARG | | 357 | −0.169 | 99.604 | −79.814 | 1.00 | 80.59 | |
| ATOM | 1506 | NE | ARG | | 357 | 1.165 | 99.077 | −80.109 | 1.00 | 83.88 | |
| ATOM | 1507 | CZ | ARG | | 357 | 1.518 | 98.504 | −81.257 | 1.00 | 84.74 | |
| ATOM | 1508 | NH1 | ARG | | 357 | 0.631 | 98.383 | −82.240 | 1.00 | 85.51 | |
| ATOM | 1509 | NH2 | ARG | | 357 | 2.754 | 98.040 | −81.417 | 1.00 | 84.34 | |
| ATOM | 1510 | C | ARG | | 357 | −2.343 | 100.077 | −75.376 | 1.00 | 67.76 | |
| ATOM | 1511 | O | ARG | | 357 | −2.035 | 101.214 | −75.021 | 1.00 | 67.91 | |
| ATOM | 1512 | N | LEU | | 358 | −3.432 | 99.457 | −74.925 | 1.00 | 63.63 | |
| ATOM | 1513 | CA | LEU | | 358 | −4.355 | 100.137 | −74.016 | 1.00 | 60.15 | |
| ATOM | 1514 | CB | LEU | | 358 | −5.553 | 99.236 | −73.669 | 1.00 | 57.26 | |
| ATOM | 1515 | CG | LEU | | 358 | −6.684 | 99.227 | −74.713 | 1.00 | 54.60 | |
| ATOM | 1516 | CD1 | LEU | | 358 | −7.908 | 98.554 | −74.161 | 1.00 | 53.63 | |
| ATOM | 1517 | CD2 | LEU | | 358 | −7.029 | 100.647 | −75.095 | 1.00 | 52.19 | |
| ATOM | 1518 | C | LEU | | 358 | −3.765 | 100.735 | −72.747 | 1.00 | 58.49 | |
| ATOM | 1519 | O | LEU | | 358 | −3.945 | 101.922 | −72.508 | 1.00 | 58.73 | |
| ATOM | 1520 | N | PHE | | 359 | −3.072 | 99.939 | −71.937 | 1.00 | 56.63 | |
| ATOM | 1521 | CA | PHE | | 359 | −2.477 | 100.452 | −70.703 | 1.00 | 54.30 | |
| ATOM | 1522 | CB | PHE | | 359 | −1.462 | 99.453 | −70.140 | 1.00 | 51.83 | |
| ATOM | 1523 | CG | PHE | | 359 | −0.861 | 99.870 | −68.828 | 1.00 | 49.28 | |
| ATOM | 1524 | CD1 | PHE | | 359 | −1.503 | 99.577 | −67.629 | 1.00 | 48.65 | |
| ATOM | 1525 | CD2 | PHE | | 359 | 0.331 | 100.583 | −68.790 | 1.00 | 48.91 | |
| ATOM | 1526 | CE1 | PHE | | 359 | −0.969 | 99.992 | −66.403 | 1.00 | 47.05 | |
| ATOM | 1527 | CE2 | PHE | | 359 | 0.880 | 101.005 | −67.568 | 1.00 | 48.51 | |
| ATOM | 1528 | CZ | PHE | | 359 | 0.225 | 100.709 | −66.372 | 1.00 | 48.33 | |
| ATOM | 1529 | C | PHE | | 359 | −1.783 | 101.802 | −70.947 | 1.00 | 54.90 | |
| ATOM | 1530 | O | PHE | | 359 | −2.099 | 102.794 | −70.288 | 1.00 | 55.02 | |
| ATOM | 1531 | N | GLU | | 360 | −0.848 | 101.846 | −71.896 | 1.00 | 55.38 | |
| ATOM | 1532 | CA | GLU | | 360 | −0.123 | 103.089 | −72.202 | 1.00 | 55.56 | |
| ATOM | 1533 | CB | GLU | | 360 | 1.065 | 102.819 | −73.136 | 1.00 | 57.15 | |
| ATOM | 1534 | CG | GLU | | 360 | 1.748 | 101.460 | −72.982 | 1.00 | 62.38 | |
| ATOM | 1535 | CD | GLU | | 360 | 1.157 | 100.399 | −73.908 | 1.00 | 65.01 | |
| ATOM | 1536 | OE1 | GLU | | 360 | 0.171 | 99.743 | −73.494 | 1.00 | 66.75 | |
| ATOM | 1537 | OE2 | GLU | | 360 | 1.667 | 100.235 | −75.050 | 1.00 | 64.31 | |
| ATOM | 1538 | C | GLU | | 360 | −1.021 | 104.158 | −72.862 | 1.00 | 54.55 | |
| ATOM | 1539 | O | GLU | | 360 | −0.868 | 105.361 | −72.626 | 1.00 | 53.90 | |
| ATOM | 1540 | N | LEU | | 361 | −1.943 | 103.716 | −73.703 | 1.00 | 53.09 | |
| ATOM | 1541 | CA | LEU | | 361 | −2.835 | 104.633 | −74.385 | 1.00 | 52.99 | |
| ATOM | 1542 | CB | LEU | | 361 | −3.697 | 103.856 | −75.381 | 1.00 | 53.03 | |
| ATOM | 1543 | CG | LEU | | 361 | −2.966 | 103.371 | −76.633 | 1.00 | 51.61 | |
| ATOM | 1544 | CD1 | LEU | | 361 | −3.926 | 102.631 | −77.512 | 1.00 | 52.09 | |
| ATOM | 1545 | CD2 | LEU | | 361 | −2.385 | 104.558 | −77.391 | 1.00 | 52.42 | |
| ATOM | 1546 | C | LEU | | 361 | −3.720 | 105.413 | −73.411 | 1.00 | 52.83 | |
| ATOM | 1547 | O | LEU | | 361 | −3.628 | 106.632 | −73.309 | 1.00 | 52.67 | |
| ATOM | 1548 | N | ILE | | 362 | −4.574 | 104.691 | −72.694 | 1.00 | 51.97 | |
| ATOM | 1549 | CA | ILE | | 362 | −5.482 | 105.283 | −71.732 | 1.00 | 50.58 | |
| ATOM | 1550 | CB | ILE | | 362 | −6.208 | 104.195 | −70.937 | 1.00 | 49.70 | |
| ATOM | 1551 | CG2 | ILE | | 362 | −7.104 | 104.819 | −69.860 | 1.00 | 47.94 | |
| ATOM | 1552 | CG1 | ILE | | 362 | −7.004 | 103.321 | −71.898 | 1.00 | 47.22 | |
| ATOM | 1553 | CD1 | ILE | | 362 | −7.508 | 102.076 | −71.252 | 1.00 | 47.62 | |
| ATOM | 1554 | C | ILE | | 362 | −4.752 | 106.171 | −70.753 | 1.00 | 51.35 | |
| ATOM | 1555 | O | ILE | | 362 | −5.290 | 107.157 | −70.285 | 1.00 | 52.23 | |
| ATOM | 1556 | N | LEU | | 363 | −3.519 | 105.835 | −70.428 | 1.00 | 51.85 | |
| ATOM | 1557 | CA | LEU | | 363 | −2.816 | 106.657 | −69.474 | 1.00 | 52.24 | |
| ATOM | 1558 | CB | LEU | | 363 | −1.749 | 105.843 | −68.760 | 1.00 | 50.66 | |
| ATOM | 1559 | CG | LEU | | 363 | −2.313 | 104.876 | −67.722 | 1.00 | 50.68 | |
| ATOM | 1560 | CD1 | LEU | | 363 | −1.325 | 103.767 | −67.441 | 1.00 | 49.35 | |
| ATOM | 1561 | CD2 | LEU | | 363 | −2.649 | 105.640 | −66.453 | 1.00 | 49.87 | |
| ATOM | 1562 | C | LEU | | 363 | −2.188 | 107.880 | −70.082 | 1.00 | 53.84 | |
| ATOM | 1563 | O | LEU | | 363 | −2.310 | 108.962 | −69.524 | 1.00 | 54.29 | |
| ATOM | 1564 | N | MET | | 364 | −1.550 | 107.729 | −71.240 | 1.00 | 55.93 | |
| ATOM | 1565 | CA | MET | | 364 | −0.835 | 108.851 | −71.848 | 1.00 | 57.84 | |
| ATOM | 1566 | CB | MET | | 364 | 0.673 | 108.561 | −71.792 | 1.00 | 57.41 | |
| ATOM | 1567 | CG | MET | | 364 | 1.113 | 107.908 | −70.495 | 1.00 | 58.69 | |
| ATOM | 1568 | SD | MET | | 364 | 2.879 | 107.503 | −70.396 | 1.00 | 61.01 | |
| ATOM | 1569 | CE | MET | | 364 | 2.879 | 105.770 | −70.850 | 1.00 | 59.26 | |
| ATOM | 1570 | C | MET | | 364 | −1.196 | 109.277 | −73.268 | 1.00 | 58.73 | |
| ATOM | 1571 | O | MET | | 364 | −0.449 | 110.014 | −73.892 | 1.00 | 60.34 | |
| ATOM | 1572 | N | GLU | | 365 | −2.322 | 108.832 | −73.791 | 1.00 | 59.20 | |
| ATOM | 1573 | CA | GLU | | 365 | −2.695 | 109.218 | −75.141 | 1.00 | 60.03 | |
| ATOM | 1574 | CB | GLU | | 365 | −2.982 | 107.977 | −75.979 | 1.00 | 61.60 | |
| ATOM | 1575 | CG | GLU | | 365 | −3.395 | 108.284 | −77.405 | 1.00 | 61.85 | |
| ATOM | 1576 | CD | GLU | | 365 | −2.208 | 108.436 | −78.298 | 1.00 | 62.17 | |
| ATOM | 1577 | OE1 | GLU | | 365 | −1.359 | 109.282 | −77.961 | 1.00 | 62.61 | |
| ATOM | 1578 | OE2 | GLU | | 365 | −2.119 | 107.712 | −79.319 | 1.00 | 63.68 | |
| ATOM | 1579 | C | GLU | | 365 | −3.935 | 110.103 | −75.138 | 1.00 | 60.53 | |
| ATOM | 1580 | O | GLU | | 365 | −4.940 | 109.775 | −74.495 | 1.00 | 59.82 | |
| ATOM | 1581 | N | GLU | | 366 | −3.871 | 111.216 | −75.866 | 1.00 | 61.00 | |
| ATOM | 1582 | CA | GLU | | 366 | −5.005 | 112.129 | −75.941 | 1.00 | 61.39 | |
| ATOM | 1583 | CB | GLU | | 366 | −4.597 | 113.450 | −76.591 | 1.00 | 63.37 | |
| ATOM | 1584 | CG | GLU | | 366 | −4.258 | 113.348 | −78.087 | 1.00 | 67.78 | |
| ATOM | 1585 | CD | GLU | | 366 | −4.383 | 114.685 | −78.820 | 1.00 | 69.43 | |
| ATOM | 1586 | OE1 | GLU | | 366 | −5.508 | 115.259 | −78.847 | 1.00 | 69.67 | |
| ATOM | 1587 | OE2 | GLU | | 366 | −3.361 | 115.157 | −79.368 | 1.00 | 69.98 | |
| ATOM | 1588 | C | GLU | | 366 | −6.120 | 111.490 | −76.760 | 1.00 | 61.39 | |
| ATOM | 1589 | O | GLU | | 366 | −5.886 | 110.976 | −77.858 | 1.00 | 60.86 | |
| ATOM | 1590 | N | ILE | | 367 | −7.328 | 111.501 | −76.213 | 1.00 | 61.54 | |
| ATOM | 1591 | CA | ILE | | 367 | −8.471 | 110.942 | −76.906 | 1.00 | 61.47 | |
| ATOM | 1592 | CB | ILE | | 367 | −9.606 | 110.686 | −75.943 | 1.00 | 60.89 | |
| ATOM | 1593 | CG2 | ILE | | 367 | −10.885 | 110.501 | −76.713 | 1.00 | 61.95 | |
| ATOM | 1594 | CG1 | ILE | | 367 | −9.261 | 109.462 | −75.081 | 1.00 | 60.19 | |
| ATOM | 1595 | CD1 | ILE | | 367 | −10.249 | 109.124 | −73.986 | 1.00 | 56.88 | |
| ATOM | 1596 | C | ILE | | 367 | −8.888 | 111.950 | −77.965 | 1.00 | 63.20 | |
| ATOM | 1597 | O | ILE | | 367 | −8.911 | 113.163 | −77.717 | 1.00 | 63.34 | |
| ATOM | 1598 | N | ARG | | 368 | −9.201 | 111.438 | −79.150 | 1.00 | 64.36 | |
| ATOM | 1599 | CA | ARG | | 368 | −9.556 | 112.264 | −80.299 | 1.00 | 65.23 | |
| ATOM | 1600 | CB | ARG | | 368 | −8.658 | 111.846 | −81.474 | 1.00 | 69.04 | |
| ATOM | 1601 | CG | ARG | | 368 | −8.856 | 112.574 | −82.790 | 1.00 | 76.89 | |
| ATOM | 1602 | CD | ARG | | 368 | −7.506 | 112.969 | −83.416 | 1.00 | 82.05 | |
| ATOM | 1603 | NE | ARG | | 368 | −6.914 | 114.116 | −82.725 | 1.00 | 87.01 | |
| ATOM | 1604 | CZ | ARG | | 368 | −6.646 | 115.294 | −83.290 | 1.00 | 88.75 | |
| ATOM | 1605 | NH1 | ARG | | 368 | −6.906 | 115.506 | −84.577 | 1.00 | 89.73 | |
| ATOM | 1606 | NH2 | ARG | | 368 | −6.133 | 116.276 | −82.557 | 1.00 | 90.35 | |
| ATOM | 1607 | C | ARG | | 368 | −11.029 | 112.091 | −80.633 | 1.00 | 63.45 | |
| ATOM | 1608 | O | ARG | | 368 | −11.560 | 110.988 | −80.515 | 1.00 | 62.89 | |
| ATOM | 1609 | N | PHE | | 369 | −11.686 | 113.177 | −81.047 | 1.00 | 62.26 | |
| ATOM | 1610 | CA | PHE | | 369 | −13.120 | 113.141 | −81.376 | 1.00 | 60.85 | |
| ATOM | 1611 | CB | PHE | | 369 | −13.894 | 114.147 | −80.521 | 1.00 | 58.47 | |
| ATOM | 1612 | CG | PHE | | 369 | −13.475 | 114.171 | −79.098 | 1.00 | 55.69 | |
| ATOM | 1613 | CD1 | PHE | | 369 | −13.898 | 113.182 | −78.224 | 1.00 | 53.89 | |
| ATOM | 1614 | CD2 | PHE | | 369 | −12.588 | 115.135 | −78.651 | 1.00 | 54.16 | |
| ATOM | 1615 | CE1 | PHE | | 369 | −13.435 | 113.147 | −76.921 | 1.00 | 53.32 | |
| ATOM | 1616 | CE2 | PHE | | 369 | −12.115 | 115.112 | −77.353 | 1.00 | 53.82 | |
| ATOM | 1617 | CZ | PHE | | 369 | −12.534 | 114.116 | −76.481 | 1.00 | 52.90 | |
| ATOM | 1618 | C | PHE | | 369 | −13.452 | 113.451 | −82.825 | 1.00 | 61.47 | |
| ATOM | 1619 | O | PHE | | 369 | −12.764 | 114.238 | −83.481 | 1.00 | 60.78 | |
| ATOM | 1620 | N | PRO | | 370 | −14.529 | 112.841 | −83.337 | 1.00 | 62.58 | |
| ATOM | 1621 | CD | PRO | | 370 | −15.245 | 111.734 | −82.686 | 1.00 | 62.83 | |
| ATOM | 1622 | CA | PRO | | 370 | −15.014 | 113.024 | −84.708 | 1.00 | 64.77 | |
| ATOM | 1623 | CB | PRO | | 370 | −16.273 | 112.165 | −84.742 | 1.00 | 63.67 | |
| ATOM | 1624 | CG | PRO | | 370 | −15.900 | 111.038 | −83.862 | 1.00 | 62.77 | |
| ATOM | 1625 | C | PRO | | 370 | −15.325 | 114.499 | −84.960 | 1.00 | 67.04 | |
| ATOM | 1626 | O | PRO | | 370 | −15.846 | 115.188 | −84.075 | 1.00 | 66.93 | |
| ATOM | 1627 | N | ARG | | 371 | −14.995 | 114.987 | −86.155 | 1.00 | 69.45 | |
| ATOM | 1628 | CA | ARG | | 371 | −15.249 | 116.383 | −86.492 | 1.00 | 70.54 | |
| ATOM | 1629 | CB | ARG | | 371 | −14.745 | 116.674 | −87.913 | 1.00 | 73.31 | |
| ATOM | 1630 | CG | ARG | | 371 | −13.262 | 116.315 | −88.161 | 1.00 | 76.94 | |
| ATOM | 1631 | CD | ARG | | 371 | −12.774 | 116.710 | −89.589 | 1.00 | 80.18 | |
| ATOM | 1632 | NE | ARG | | 371 | −13.485 | 115.988 | −90.653 | 1.00 | 83.53 | |
| ATOM | 1633 | CZ | ARG | | 371 | −13.091 | 114.836 | −91.205 | 1.00 | 85.07 | |
| ATOM | 1634 | NH1 | ARG | | 371 | −11.963 | 114.238 | −90.822 | 1.00 | 85.98 | |
| ATOM | 1635 | NH2 | ARG | | 371 | −13.854 | 114.256 | −92.126 | 1.00 | 84.82 | |
| ATOM | 1636 | C | ARG | | 371 | −16.762 | 116.628 | −86.376 | 1.00 | 69.82 | |
| ATOM | 1637 | O | ARG | | 371 | −17.203 | 117.636 | −85.832 | 1.00 | 68.81 | |
| ATOM | 1638 | N | THR | | 372 | −17.538 | 115.665 | −86.862 | 1.00 | 69.75 | |
| ATOM | 1639 | CA | THR | | 372 | −19.003 | 115.694 | −86.841 | 1.00 | 71.11 | |
| ATOM | 1640 | CB | THR | | 372 | −19.524 | 114.599 | −87.794 | 1.00 | 71.17 | |
| ATOM | 1641 | OG1 | THR | | 372 | −20.880 | 114.278 | −87.476 | 1.00 | 73.09 | |
| ATOM | 1642 | CG2 | THR | | 372 | −18.670 | 113.334 | −87.666 | 1.00 | 71.70 | |
| ATOM | 1643 | C | THR | | 372 | −19.619 | 115.477 | −85.428 | 1.00 | 71.80 | |
| ATOM | 1644 | O | THR | | 372 | −20.414 | 114.547 | −85.219 | 1.00 | 72.12 | |
| ATOM | 1645 | N | LEU | | 373 | −19.276 | 116.342 | −84.469 | 1.00 | 71.42 | |
| ATOM | 1646 | CA | LEU | | 373 | −19.758 | 116.190 | −83.089 | 1.00 | 69.80 | |
| ATOM | 1647 | CB | LEU | | 373 | −18.698 | 115.433 | −82.279 | 1.00 | 70.86 | |
| ATOM | 1648 | CG | LEU | | 373 | −19.076 | 114.509 | −81.117 | 1.00 | 71.23 | |
| ATOM | 1649 | CD1 | LEU | | 373 | −19.837 | 113.303 | −81.653 | 1.00 | 70.31 | |
| ATOM | 1650 | CD2 | LEU | | 373 | −17.816 | 114.061 | −80.392 | 1.00 | 70.52 | |
| ATOM | 1651 | C | LEU | | 373 | −19.994 | 117.558 | −82.455 | 1.00 | 68.53 | |
| ATOM | 1652 | O | LEU | | 373 | −19.044 | 118.321 | −82.279 | 1.00 | 68.27 | |
| ATOM | 1653 | N | SER | | 374 | −21.241 | 117.860 | −82.098 | 1.00 | 67.32 | |
| ATOM | 1654 | CA | SER | | 374 | −21.571 | 119.166 | −81.514 | 1.00 | 67.55 | |
| ATOM | 1655 | CB | SER | | 374 | −22.919 | 119.115 | −80.797 | 1.00 | 66.97 | |
| ATOM | 1656 | OG | SER | | 374 | −22.788 | 118.583 | −79.489 | 1.00 | 67.60 | |
| ATOM | 1657 | C | SER | | 374 | −20.486 | 119.627 | −80.541 | 1.00 | 67.21 | |
| ATOM | 1658 | O | SER | | 374 | −20.059 | 118.883 | −79.669 | 1.00 | 69.14 | |
| ATOM | 1659 | N | PRO | | 375 | −20.037 | 120.874 | −80.668 | 1.00 | 65.82 | |
| ATOM | 1660 | CD | PRO | | 375 | −20.692 | 121.978 | −81.380 | 1.00 | 65.36 | |
| ATOM | 1661 | CA | PRO | | 375 | −18.985 | 121.376 | −79.773 | 1.00 | 64.80 | |
| ATOM | 1662 | CB | PRO | | 375 | −18.886 | 122.853 | −80.149 | 1.00 | 64.82 | |
| ATOM | 1663 | CG | PRO | | 375 | −20.300 | 123.170 | −80.532 | 1.00 | 66.04 | |
| ATOM | 1664 | C | PRO | | 375 | −19.229 | 121.160 | −78.280 | 1.00 | 63.31 | |
| ATOM | 1665 | O | PRO | | 375 | −18.268 | 121.163 | −77.489 | 1.00 | 61.84 | |
| ATOM | 1666 | N | GLU | | 376 | −20.497 | 120.987 | −77.892 | 1.00 | 61.85 | |
| ATOM | 1667 | CA | GLU | | 376 | −20.819 | 120.753 | −76.480 | 1.00 | 62.01 | |
| ATOM | 1668 | CB | GLU | | 376 | −22.276 | 121.106 | −76.147 | 1.00 | 61.80 | |
| ATOM | 1669 | CG | GLU | | 376 | −23.296 | 120.418 | −77.005 | 1.00 | 64.69 | |
| ATOM | 1670 | CD | GLU | | 376 | −23.834 | 121.325 | −78.113 | 1.00 | 66.85 | |
| ATOM | 1671 | OE1 | GLU | | 376 | −23.108 | 122.248 | −78.564 | 1.00 | 67.43 | |
| ATOM | 1672 | OE2 | GLU | | 376 | −24.983 | 121.095 | −78.546 | 1.00 | 65.89 | |
| ATOM | 1673 | C | GLU | | 376 | −20.547 | 119.286 | −76.162 | 1.00 | 61.28 | |
| ATOM | 1674 | O | GLU | | 376 | −19.997 | 118.973 | −75.094 | 1.00 | 61.94 | |
| ATOM | 1675 | N | ALA | | 377 | −20.924 | 118.392 | −77.082 | 1.00 | 58.93 | |
| ATOM | 1676 | CA | ALA | | 377 | −20.663 | 116.957 | −76.912 | 1.00 | 56.68 | |
| ATOM | 1677 | CB | ALA | | 377 | −21.267 | 116.186 | −78.065 | 1.00 | 55.69 | |
| ATOM | 1678 | C | ALA | | 377 | −19.135 | 116.717 | −76.839 | 1.00 | 55.03 | |
| ATOM | 1679 | O | ALA | | 377 | −18.647 | 115.929 | −76.038 | 1.00 | 52.86 | |
| ATOM | 1680 | N | LYS | | 378 | −18.386 | 117.411 | −77.682 | 1.00 | 54.47 | |
| ATOM | 1681 | CA | LYS | | 378 | −16.946 | 117.290 | −77.660 | 1.00 | 54.99 | |
| ATOM | 1682 | CB | LYS | | 378 | −16.322 | 118.023 | −78.868 | 1.00 | 57.80 | |
| ATOM | 1683 | CG | LYS | | 378 | −14.778 | 118.182 | −78.821 | 1.00 | 61.35 | |
| ATOM | 1684 | CD | LYS | | 378 | −14.214 | 118.776 | −80.137 | 1.00 | 64.11 | |
| ATOM | 1685 | CE | LYS | | 378 | −12.696 | 119.099 | −80.062 | 1.00 | 65.84 | |
| ATOM | 1686 | NZ | LYS | | 378 | −12.139 | 119.690 | −81.333 | 1.00 | 64.38 | |
| ATOM | 1687 | C | LYS | | 378 | −16.492 | 117.928 | −76.352 | 1.00 | 53.95 | |
| ATOM | 1688 | O | LYS | | 378 | −15.357 | 117.731 | −75.901 | 1.00 | 54.88 | |
| ATOM | 1689 | N | SER | | 379 | −17.391 | 118.682 | −75.731 | 1.00 | 52.17 | |
| ATOM | 1690 | CA | SER | | 379 | −17.064 | 119.352 | −74.475 | 1.00 | 49.37 | |
| ATOM | 1691 | CB | SER | | 379 | −18.011 | 120.534 | −74.224 | 1.00 | 48.67 | |
| ATOM | 1692 | OG | SER | | 379 | −17.529 | 121.359 | −73.169 | 1.00 | 47.44 | |
| ATOM | 1693 | C | SER | | 379 | −17.171 | 118.364 | −73.328 | 1.00 | 47.02 | |
| ATOM | 1694 | O | SER | | 379 | −16.280 | 118.274 | −72.479 | 1.00 | 43.80 | |
| ATOM | 1695 | N | LEU | | 380 | −18.284 | 117.639 | −73.329 | 1.00 | 45.31 | |
| ATOM | 1696 | CA | LEU | | 380 | −18.589 | 116.632 | −72.330 | 1.00 | 45.01 | |
| ATOM | 1697 | CB | LEU | | 380 | −19.936 | 115.981 | −72.677 | 1.00 | 42.73 | |
| ATOM | 1698 | CG | LEU | | 380 | −20.567 | 115.047 | −71.650 | 1.00 | 42.08 | |
| ATOM | 1699 | CD1 | LEU | | 380 | −20.777 | 115.803 | −70.344 | 1.00 | 39.85 | |
| ATOM | 1700 | CD2 | LEU | | 380 | −21.873 | 114.459 | −72.186 | 1.00 | 40.06 | |
| ATOM | 1701 | C | LEU | | 380 | −17.472 | 115.570 | −72.287 | 1.00 | 45.96 | |
| ATOM | 1702 | O | LEU | | 380 | −16.728 | 115.449 | −71.286 | 1.00 | 45.21 | |
| ATOM | 1703 | N | LEU | | 381 | −17.348 | 114.806 | −73.368 | 1.00 | 44.53 | |
| ATOM | 1704 | CA | LEU | | 381 | −16.328 | 113.774 | −73.419 | 1.00 | 43.33 | |
| ATOM | 1705 | CB | LEU | | 381 | −16.241 | 113.199 | −74.802 | 1.00 | 41.46 | |
| ATOM | 1706 | CG | LEU | | 381 | −17.643 | 112.869 | −75.242 | 1.00 | 41.01 | |
| ATOM | 1707 | CD1 | LEU | | 381 | −17.614 | 112.364 | −76.674 | 1.00 | 39.08 | |
| ATOM | 1708 | CD2 | LEU | | 381 | −18.233 | 111.876 | −74.270 | 1.00 | 40.64 | |
| ATOM | 1709 | C | LEU | | 381 | −14.978 | 114.315 | −73.020 | 1.00 | 43.61 | |
| ATOM | 1710 | O | LEU | | 381 | −14.323 | 113.788 | −72.128 | 1.00 | 43.42 | |
| ATOM | 1711 | N | ALA | | 382 | −14.540 | 115.372 | −73.672 | 1.00 | 44.81 | |
| ATOM | 1712 | CA | ALA | | 382 | −13.243 | 115.903 | −73.307 | 1.00 | 46.42 | |
| ATOM | 1713 | CB | ALA | | 382 | −13.042 | 117.276 | −73.939 | 1.00 | 46.11 | |
| ATOM | 1714 | C | ALA | | 382 | −13.151 | 115.989 | −71.781 | 1.00 | 46.76 | |
| ATOM | 1715 | O | ALA | | 382 | −12.072 | 115.836 | −71.212 | 1.00 | 47.69 | |
| ATOM | 1716 | N | GLY | | 383 | −14.302 | 116.178 | −71.129 | 1.00 | 46.94 | |
| ATOM | 1717 | CA | GLY | | 383 | −14.339 | 116.330 | −69.674 | 1.00 | 46.89 | |
| ATOM | 1718 | C | GLY | | 383 | −14.605 | 115.087 | −68.843 | 1.00 | 46.16 | |
| ATOM | 1719 | O | GLY | | 383 | −14.119 | 114.944 | −67.715 | 1.00 | 44.69 | |
| ATOM | 1720 | N | LEU | | 384 | −15.409 | 114.190 | −69.380 | 1.00 | 44.84 | |
| ATOM | 1721 | CA | LEU | | 384 | −15.660 | 112.970 | −68.663 | 1.00 | 44.28 | |
| ATOM | 1722 | CB | LEU | | 384 | −16.835 | 112.218 | −69.295 | 1.00 | 42.32 | |
| ATOM | 1723 | CG | LEU | | 384 | −18.192 | 112.901 | −69.109 | 1.00 | 41.84 | |
| ATOM | 1724 | CD1 | LEU | | 384 | −19.295 | 112.121 | −69.807 | 1.00 | 39.23 | |
| ATOM | 1725 | CD2 | LEU | | 384 | −18.475 | 113.001 | −67.611 | 1.00 | 40.93 | |
| ATOM | 1726 | C | LEU | | 384 | −14.395 | 112.124 | −68.735 | 1.00 | 45.06 | |
| ATOM | 1727 | O | LEU | | 384 | −14.207 | 111.236 | −67.915 | 1.00 | 46.62 | |
| ATOM | 1728 | N | LEU | | 385 | −13.519 | 112.430 | −69.695 | 1.00 | 44.98 | |
| ATOM | 1729 | CA | LEU | | 385 | −12.294 | 111.653 | −69.921 | 1.00 | 43.46 | |
| ATOM | 1730 | CB | LEU | | 385 | −12.198 | 111.267 | −71.385 | 1.00 | 40.19 | |
| ATOM | 1731 | CG | LEU | | 385 | −13.448 | 110.502 | −71.784 | 1.00 | 40.02 | |
| ATOM | 1732 | CD1 | LEU | | 385 | −13.576 | 110.453 | −73.302 | 1.00 | 39.91 | |
| ATOM | 1733 | CD2 | LEU | | 385 | −13.391 | 109.138 | −71.159 | 1.00 | 40.44 | |
| ATOM | 1734 | C | LEU | | 385 | −10.963 | 112.240 | −69.485 | 1.00 | 43.65 | |
| ATOM | 1735 | O | LEU | | 385 | −9.908 | 111.747 | −69.880 | 1.00 | 43.05 | |
| ATOM | 1736 | N | LYS | | 386 | −10.999 | 113.295 | −68.685 | 1.00 | 44.64 | |
| ATOM | 1737 | CA | LYS | | 386 | −9.765 | 113.861 | −68.183 | 1.00 | 46.79 | |
| ATOM | 1738 | CB | LYS | | 386 | −10.074 | 115.018 | −67.227 | 1.00 | 48.36 | |
| ATOM | 1739 | CG | LYS | | 386 | −10.220 | 116.375 | −67.909 | 1.00 | 52.10 | |
| ATOM | 1740 | CD | LYS | | 386 | −8.891 | 116.759 | −68.584 | 1.00 | 56.86 | |
| ATOM | 1741 | CE | LYS | | 386 | −8.843 | 118.214 | −69.092 | 1.00 | 58.05 | |
| ATOM | 1742 | NZ | LYS | | 386 | −8.448 | 119.203 | −68.027 | 1.00 | 59.99 | |
| ATOM | 1743 | C | LYS | | 386 | −9.035 | 112.718 | −67.458 | 1.00 | 47.37 | |
| ATOM | 1744 | O | LYS | | 386 | −9.620 | 112.013 | −66.640 | 1.00 | 47.60 | |
| ATOM | 1745 | N | LYS | | 387 | −7.764 | 112.519 | −67.767 | 1.00 | 47.79 | |
| ATOM | 1746 | CA | LYS | | 387 | −7.033 | 111.439 | −67.128 | 1.00 | 48.42 | |
| ATOM | 1747 | CB | LYS | | 387 | −5.699 | 111.213 | −67.843 | 1.00 | 48.01 | |
| ATOM | 1748 | CG | LYS | | 387 | −5.875 | 110.641 | −69.243 | 1.00 | 46.81 | |
| ATOM | 1749 | CD | LYS | | 387 | −4.663 | 110.912 | −70.117 | 1.00 | 46.36 | |
| ATOM | 1750 | CE | LYS | | 387 | −4.658 | 110.026 | −71.359 | 1.00 | 46.26 | |
| ATOM | 1751 | NZ | LYS | | 387 | −5.956 | 109.963 | −72.100 | 1.00 | 44.08 | |
| ATOM | 1752 | C | LYS | | 387 | −6.819 | 111.711 | −65.651 | 1.00 | 49.30 | |
| ATOM | 1753 | O | LYS | | 387 | −6.690 | 110.794 | −64.850 | 1.00 | 48.51 | |
| ATOM | 1754 | N | ASP | | 388 | −6.789 | 112.976 | −65.274 | 1.00 | 51.05 | |
| ATOM | 1755 | CA | ASP | | 388 | −6.610 | 113.289 | −63.866 | 1.00 | 53.06 | |
| ATOM | 1756 | CB | ASP | | 388 | −5.966 | 114.661 | −63.706 | 1.00 | 53.15 | |
| ATOM | 1757 | CG | ASP | | 388 | −5.602 | 114.960 | −62.277 | 1.00 | 53.88 | |
| ATOM | 1758 | OD1 | ASP | | 388 | −6.273 | 114.411 | −61.382 | 1.00 | 54.59 | |
| ATOM | 1759 | OD2 | ASP | | 388 | −4.662 | 115.750 | −62.045 | 1.00 | 55.38 | |
| ATOM | 1760 | C | ASP | | 388 | −7.988 | 113.269 | −63.194 | 1.00 | 54.47 | |
| ATOM | 1761 | O | ASP | | 388 | −8.852 | 114.085 | −63.503 | 1.00 | 54.45 | |
| ATOM | 1762 | N | PRO | | 389 | −8.218 | 112.316 | −62.280 | 1.00 | 56.07 | |
| ATOM | 1763 | CD | PRO | | 389 | −7.371 | 111.211 | −61.798 | 1.00 | 56.72 | |
| ATOM | 1764 | CA | PRO | | 389 | −9.533 | 112.289 | −61.634 | 1.00 | 56.52 | |
| ATOM | 1765 | CB | PRO | | 389 | −9.400 | 111.154 | −60.607 | 1.00 | 55.95 | |
| ATOM | 1766 | CG | PRO | | 389 | −7.904 | 111.008 | −60.409 | 1.00 | 56.22 | |
| ATOM | 1767 | C | PRO | | 389 | −9.907 | 113.625 | −61.010 | 1.00 | 56.98 | |
| ATOM | 1768 | O | PRO | | 389 | −11.050 | 114.073 | −61.126 | 1.00 | 56.98 | |
| ATOM | 1769 | N | LYS | | 390 | −8.936 | 114.264 | −60.368 | 1.00 | 57.25 | |
| ATOM | 1770 | CA | LYS | | 390 | −9.160 | 115.553 | −59.732 | 1.00 | 58.61 | |
| ATOM | 1771 | CB | LYS | | 390 | −7.867 | 116.073 | −59.108 | 1.00 | 60.51 | |
| ATOM | 1772 | CG | LYS | | 390 | −7.667 | 115.682 | −57.649 | 1.00 | 63.58 | |
| ATOM | 1773 | CD | LYS | | 390 | −6.332 | 116.196 | −57.102 | 1.00 | 66.33 | |
| ATOM | 1774 | CE | LYS | | 390 | −6.366 | 116.325 | −55.575 | 1.00 | 69.42 | |
| ATOM | 1775 | NZ | LYS | | 390 | −6.791 | 115.062 | −54.860 | 1.00 | 71.82 | |
| ATOM | 1776 | C | LYS | | 390 | −9.688 | 116.577 | −60.721 | 1.00 | 58.91 | |
| ATOM | 1777 | O | LYS | | 390 | −10.438 | 117.490 | −60.350 | 1.00 | 59.35 | |
| ATOM | 1778 | N | GLN | | 391 | −9.309 | 116.428 | −61.983 | 1.00 | 58.86 | |
| ATOM | 1779 | CA | GLN | | 391 | −9.765 | 117.365 | −62.996 | 1.00 | 58.63 | |
| ATOM | 1780 | CB | GLN | | 391 | −8.660 | 117.655 | −64.003 | 1.00 | 61.12 | |
| ATOM | 1781 | CG | GLN | | 391 | −7.457 | 118.404 | −63.457 | 1.00 | 65.85 | |
| ATOM | 1782 | CD | GLN | | 391 | −6.403 | 118.596 | −64.542 | 1.00 | 70.16 | |
| ATOM | 1783 | OE1 | GLN | | 391 | −6.587 | 119.394 | −65.480 | 1.00 | 71.02 | |
| ATOM | 1784 | NE2 | GLN | | 391 | −5.301 | 117.841 | −64.440 | 1.00 | 70.27 | |
| ATOM | 1785 | C | GLN | | 391 | −11.003 | 116.932 | −63.760 | 1.00 | 57.14 | |
| ATOM | 1786 | O | GLN | | 391 | −11.706 | 117.778 | −64.288 | 1.00 | 59.42 | |
| ATOM | 1787 | N | ARG | | 392 | −11.287 | 115.638 | −63.831 | 1.00 | 53.89 | |
| ATOM | 1788 | CA | ARG | | 392 | −12.459 | 115.191 | −64.581 | 1.00 | 52.12 | |
| ATOM | 1789 | CB | ARG | | 392 | −12.671 | 113.705 | −64.375 | 1.00 | 51.68 | |
| ATOM | 1790 | CG | ARG | | 392 | −13.589 | 113.031 | −65.379 | 1.00 | 51.30 | |
| ATOM | 1791 | CD | ARG | | 392 | −13.307 | 111.535 | −65.331 | 1.00 | 51.76 | |
| ATOM | 1792 | NE | ARG | | 392 | −11.858 | 111.316 | −65.235 | 1.00 | 49.83 | |
| ATOM | 1793 | CZ | ARG | | 392 | −11.281 | 110.411 | −64.457 | 1.00 | 47.76 | |
| ATOM | 1794 | NH1 | ARG | | 392 | −12.027 | 109.618 | −63.703 | 1.00 | 46.43 | |
| ATOM | 1795 | NH2 | ARG | | 392 | −9.958 | 110.331 | −64.408 | 1.00 | 47.65 | |
| ATOM | 1796 | C | ARG | | 392 | −13.761 | 115.923 | −64.241 | 1.00 | 51.08 | |
| ATOM | 1797 | O | ARG | | 392 | −13.915 | 116.485 | −63.162 | 1.00 | 50.36 | |
| ATOM | 1798 | N | LEU | | 393 | −14.693 | 115.908 | −65.186 | 1.00 | 49.96 | |
| ATOM | 1799 | CA | LEU | | 393 | −15.986 | 116.542 | −65.014 | 1.00 | 49.55 | |
| ATOM | 1800 | CB | LEU | | 393 | −16.672 | 116.669 | −66.377 | 1.00 | 48.34 | |
| ATOM | 1801 | CG | LEU | | 393 | −17.687 | 117.778 | −66.684 | 1.00 | 49.19 | |
| ATOM | 1802 | CD1 | LEU | | 393 | −18.839 | 117.184 | −67.497 | 1.00 | 49.83 | |
| ATOM | 1803 | CD2 | LEU | | 393 | −18.214 | 118.417 | −65.412 | 1.00 | 50.60 | |
| ATOM | 1804 | C | LEU | | 393 | −16.796 | 115.617 | −64.096 | 1.00 | 51.24 | |
| ATOM | 1805 | O | LEU | | 393 | −17.141 | 114.494 | −64.479 | 1.00 | 52.00 | |
| ATOM | 1806 | N | GLY | | 394 | −17.113 | 116.078 | −62.891 | 1.00 | 51.09 | |
| ATOM | 1807 | CA | GLY | | 394 | −17.863 | 115.229 | −61.975 | 1.00 | 50.50 | |
| ATOM | 1808 | C | GLY | | 394 | −16.866 | 114.686 | −60.974 | 1.00 | 49.64 | |
| ATOM | 1809 | O | GLY | | 394 | −17.197 | 113.919 | −60.075 | 1.00 | 47.57 | |
| ATOM | 1810 | N | GLY | | 395 | −15.624 | 115.128 | −61.155 | 1.00 | 49.62 | |
| ATOM | 1811 | CA | GLY | | 395 | −14.533 | 114.726 | −60.297 | 1.00 | 50.44 | |
| ATOM | 1812 | C | GLY | | 395 | −14.325 | 115.762 | −59.227 | 1.00 | 51.76 | |
| ATOM | 1813 | O | GLY | | 395 | −13.457 | 115.592 | −58.367 | 1.00 | 52.68 | |
| ATOM | 1814 | N | GLY | | 396 | −15.118 | 116.836 | −59.281 | 1.00 | 52.01 | |
| ATOM | 1815 | CA | GLY | | 396 | −15.034 | 117.897 | −58.286 | 1.00 | 53.00 | |
| ATOM | 1816 | C | GLY | | 396 | −15.975 | 117.650 | −57.116 | 1.00 | 53.59 | |
| ATOM | 1817 | O | GLY | | 396 | −16.932 | 116.895 | −57.252 | 1.00 | 54.24 | |
| ATOM | 1818 | N | PRO | | 397 | −15.751 | 118.291 | −55.959 | 1.00 | 54.28 | |
| ATOM | 1819 | CD | PRO | | 397 | −14.835 | 119.434 | −55.823 | 1.00 | 54.32 | |
| ATOM | 1820 | CA | PRO | | 397 | −16.577 | 118.141 | −54.747 | 1.00 | 54.47 | |
| ATOM | 1821 | CB | PRO | | 397 | −16.054 | 119.245 | −53.836 | 1.00 | 54.97 | |
| ATOM | 1822 | CG | PRO | | 397 | −15.549 | 120.278 | −54.813 | 1.00 | 55.09 | |
| ATOM | 1823 | C | PRO | | 397 | −18.093 | 118.225 | −54.954 | 1.00 | 55.41 | |
| ATOM | 1824 | O | PRO | | 397 | −18.874 | 117.742 | −54.122 | 1.00 | 56.14 | |
| ATOM | 1825 | N | SER | | 398 | −18.500 | 118.830 | −56.070 | 1.00 | 55.16 | |
| ATOM | 1826 | CA | SER | | 398 | −19.905 | 118.972 | −56.425 | 1.00 | 52.76 | |
| ATOM | 1827 | CB | SER | | 398 | −20.077 | 120.149 | −57.376 | 1.00 | 54.19 | |
| ATOM | 1828 | OG | SER | | 398 | −21.404 | 120.170 | −57.885 | 1.00 | 57.17 | |
| ATOM | 1829 | C | SER | | 398 | −20.448 | 117.717 | −57.109 | 1.00 | 51.78 | |
| ATOM | 1830 | O | SER | | 398 | −21.658 | 117.604 | −57.355 | 1.00 | 52.25 | |
| ATOM | 1831 | N | ASP | | 399 | −19.543 | 116.801 | −57.453 | 1.00 | 49.40 | |
| ATOM | 1832 | CA | ASP | | 399 | −19.888 | 115.534 | −58.102 | 1.00 | 47.59 | |
| ATOM | 1833 | CB | ASP | | 399 | −20.400 | 114.521 | −57.050 | 1.00 | 48.09 | |
| ATOM | 1834 | CG | ASP | | 399 | −20.297 | 113.066 | −57.521 | 1.00 | 48.96 | |
| ATOM | 1835 | OD1 | ASP | | 399 | −21.344 | 112.440 | −57.792 | 1.00 | 48.69 | |
| ATOM | 1836 | OD2 | ASP | | 399 | −19.161 | 112.543 | −57.620 | 1.00 | 49.08 | |
| ATOM | 1837 | C | ASP | | 399 | −20.897 | 115.692 | −59.239 | 1.00 | 45.55 | |
| ATOM | 1838 | O | ASP | | 399 | −20.773 | 116.589 | −60.047 | 1.00 | 45.59 | |
| ATOM | 1839 | N | ALA | | 400 | −21.896 | 114.823 | −59.288 | 1.00 | 45.15 | |
| ATOM | 1840 | CA | ALA | | 400 | −22.896 | 114.842 | −60.346 | 1.00 | 46.94 | |
| ATOM | 1841 | CB | ALA | | 400 | −24.009 | 113.870 | −60.012 | 1.00 | 43.68 | |
| ATOM | 1842 | C | ALA | | 400 | −23.497 | 116.199 | −60.740 | 1.00 | 49.95 | |
| ATOM | 1843 | O | ALA | | 400 | −24.223 | 116.293 | −61.745 | 1.00 | 51.57 | |
| ATOM | 1844 | N | LYS | | 401 | −23.228 | 117.258 | −59.981 | 1.00 | 52.07 | |
| ATOM | 1845 | CA | LYS | | 401 | −23.809 | 118.528 | −60.378 | 1.00 | 53.83 | |
| ATOM | 1846 | CB | LYS | | 401 | −23.809 | 119.518 | −59.225 | 1.00 | 55.19 | |
| ATOM | 1847 | CG | LYS | | 401 | −25.013 | 119.298 | −58.315 | 1.00 | 59.36 | |
| ATOM | 1848 | CD | LYS | | 401 | −24.866 | 120.046 | −56.995 | 1.00 | 61.63 | |
| ATOM | 1849 | CE | LYS | | 401 | −25.722 | 119.436 | −55.884 | 1.00 | 61.07 | |
| ATOM | 1850 | NZ | LYS | | 401 | −25.331 | 120.004 | −54.554 | 1.00 | 60.54 | |
| ATOM | 1851 | C | LYS | | 401 | −23.073 | 119.067 | −61.577 | 1.00 | 53.82 | |
| ATOM | 1852 | O | LYS | | 401 | −23.686 | 119.535 | −62.528 | 1.00 | 54.37 | |
| ATOM | 1853 | N | GLU | | 402 | −21.757 | 118.971 | −61.553 | 1.00 | 53.25 | |
| ATOM | 1854 | CA | GLU | | 402 | −20.984 | 119.444 | −62.676 | 1.00 | 53.58 | |
| ATOM | 1855 | CB | GLU | | 402 | −19.504 | 119.147 | −62.445 | 1.00 | 54.86 | |
| ATOM | 1856 | CG | GLU | | 402 | −18.880 | 120.014 | −61.379 | 1.00 | 57.21 | |
| ATOM | 1857 | CD | GLU | | 402 | −17.666 | 119.380 | −60.726 | 1.00 | 60.57 | |
| ATOM | 1858 | OE1 | GLU | | 402 | −16.808 | 118.836 | −61.461 | 1.00 | 61.15 | |
| ATOM | 1859 | OE2 | GLU | | 402 | −17.568 | 119.436 | −59.473 | 1.00 | 61.74 | |
| ATOM | 1860 | C | GLU | | 402 | −21.493 | 118.761 | −63.950 | 1.00 | 53.81 | |
| ATOM | 1861 | O | GLU | | 402 | −21.674 | 119.407 | −64.983 | 1.00 | 54.59 | |
| ATOM | 1862 | N | VAL | | 403 | −21.765 | 117.466 | −63.881 | 1.00 | 53.03 | |
| ATOM | 1863 | CA | VAL | | 403 | −22.233 | 116.773 | −65.073 | 1.00 | 52.97 | |
| ATOM | 1864 | CB | VAL | | 403 | −22.083 | 115.243 | −64.907 | 1.00 | 50.93 | |
| ATOM | 1865 | CG1 | VAL | | 403 | −22.741 | 114.491 | −66.070 | 1.00 | 47.49 | |
| ATOM | 1866 | CG2 | VAL | | 403 | −20.601 | 114.908 | −64.814 | 1.00 | 49.93 | |
| ATOM | 1867 | C | VAL | | 403 | −23.670 | 117.137 | −65.458 | 1.00 | 54.53 | |
| ATOM | 1868 | O | VAL | | 403 | −24.008 | 117.206 | −66.648 | 1.00 | 55.78 | |
| ATOM | 1869 | N | MET | | 404 | −24.513 | 117.382 | −64.462 | 1.00 | 55.12 | |
| ATOM | 1870 | CA | MET | | 404 | −25.903 | 117.741 | −64.728 | 1.00 | 56.16 | |
| ATOM | 1871 | CB | MET | | 404 | −26.717 | 117.629 | −63.446 | 1.00 | 56.98 | |
| ATOM | 1872 | CG | MET | | 404 | −26.993 | 116.206 | −63.038 | 1.00 | 57.98 | |
| ATOM | 1873 | SD | MET | | 404 | −27.908 | 116.121 | −61.526 | 1.00 | 59.07 | |
| ATOM | 1874 | CE | MET | | 404 | −26.647 | 116.771 | −60.384 | 1.00 | 57.66 | |
| ATOM | 1875 | C | MET | | 404 | −26.006 | 119.154 | −65.274 | 1.00 | 56.24 | |
| ATOM | 1876 | O | MET | | 404 | −26.874 | 119.461 | −66.088 | 1.00 | 54.90 | |
| ATOM | 1877 | N | GLU | | 405 | −25.097 | 120.000 | −64.807 | 1.00 | 56.49 | |
| ATOM | 1878 | CA | GLU | | 405 | −25.030 | 121.392 | −65.193 | 1.00 | 57.03 | |
| ATOM | 1879 | CB | GLU | | 405 | −24.706 | 122.249 | −63.982 | 1.00 | 57.28 | |
| ATOM | 1880 | CG | GLU | | 405 | −25.876 | 122.502 | −63.088 | 1.00 | 60.66 | |
| ATOM | 1881 | CD | GLU | | 405 | −25.431 | 122.776 | −61.688 | 1.00 | 62.93 | |
| ATOM | 1882 | OE1 | GLU | | 405 | −24.283 | 123.254 | −61.539 | 1.00 | 61.54 | |
| ATOM | 1883 | OE2 | GLU | | 405 | −26.219 | 122.513 | −60.747 | 1.00 | 65.44 | |
| ATOM | 1884 | C | GLU | | 405 | −23.967 | 121.637 | −66.236 | 1.00 | 58.00 | |
| ATOM | 1885 | O | GLU | | 405 | −23.217 | 122.623 | −66.141 | 1.00 | 59.39 | |
| ATOM | 1886 | N | HIS | | 406 | −23.858 | 120.746 | −67.211 | 1.00 | 56.91 | |
| ATOM | 1887 | CA | HIS | | 406 | −22.873 | 120.962 | −68.248 | 1.00 | 55.53 | |
| ATOM | 1888 | CB | HIS | | 406 | −22.061 | 119.699 | −68.550 | 1.00 | 53.24 | |
| ATOM | 1889 | CG | HIS | | 406 | −21.139 | 119.838 | −69.729 | 1.00 | 50.16 | |
| ATOM | 1890 | CD2 | HIS | | 406 | −19.831 | 120.192 | −69.804 | 1.00 | 48.59 | |
| ATOM | 1891 | ND1 | HIS | | 406 | −21.557 | 119.642 | −71.034 | 1.00 | 49.62 | |
| ATOM | 1892 | CE1 | HIS | | 406 | −20.546 | 119.870 | −71.858 | 1.00 | 48.28 | |
| ATOM | 1893 | NE2 | HIS | | 406 | −19.487 | 120.206 | −71.139 | 1.00 | 48.59 | |
| ATOM | 1894 | C | HIS | | 406 | −23.613 | 121.396 | −69.479 | 1.00 | 55.78 | |
| ATOM | 1895 | O | HIS | | 406 | −24.787 | 121.071 | −69.668 | 1.00 | 53.92 | |
| ATOM | 1896 | N | ARG | | 407 | −22.904 | 122.168 | −70.293 | 1.00 | 57.33 | |
| ATOM | 1897 | CA | ARG | | 407 | −23.401 | 122.697 | −71.551 | 1.00 | 57.68 | |
| ATOM | 1898 | CB | ARG | | 407 | −22.182 | 123.067 | −72.402 | 1.00 | 61.12 | |
| ATOM | 1899 | CG | ARG | | 407 | −22.411 | 123.709 | −73.766 | 1.00 | 66.54 | |
| ATOM | 1900 | CD | ARG | | 407 | −21.026 | 123.834 | −74.450 | 1.00 | 72.28 | |
| ATOM | 1901 | NE | ARG | | 407 | −20.978 | 124.757 | −75.594 | 1.00 | 77.56 | |
| ATOM | 1902 | CZ | ARG | | 407 | −19.851 | 125.217 | −76.156 | 1.00 | 79.95 | |
| ATOM | 1903 | NH1 | ARG | | 407 | −18.653 | 124.849 | −75.690 | 1.00 | 79.70 | |
| ATOM | 1904 | NH2 | ARG | | 407 | −19.920 | 126.058 | −77.189 | 1.00 | 80.94 | |
| ATOM | 1905 | C | ARG | | 407 | −24.270 | 121.635 | −72.233 | 1.00 | 56.17 | |
| ATOM | 1906 | O | ARG | | 407 | −25.441 | 121.882 | −72.544 | 1.00 | 56.34 | |
| ATOM | 1907 | N | PHE | | 408 | −23.701 | 120.442 | −72.418 | 1.00 | 53.99 | |
| ATOM | 1908 | CA | PHE | | 408 | −24.394 | 119.336 | −73.087 | 1.00 | 51.30 | |
| ATOM | 1909 | CB | PHE | | 408 | −23.521 | 118.053 | −73.063 | 1.00 | 47.78 | |
| ATOM | 1910 | CG | PHE | | 408 | −24.156 | 116.874 | −73.782 | 1.00 | 43.10 | |
| ATOM | 1911 | CD1 | PHE | | 408 | −24.176 | 116.820 | −75.176 | 1.00 | 40.46 | |
| ATOM | 1912 | CD2 | PHE | | 408 | −24.837 | 115.879 | −73.061 | 1.00 | 39.68 | |
| ATOM | 1913 | CE1 | PHE | | 408 | −24.871 | 115.798 | −75.852 | 1.00 | 40.23 | |
| ATOM | 1914 | CE2 | PHE | | 408 | −25.537 | 114.856 | −73.725 | 1.00 | 39.16 | |
| ATOM | 1915 | CZ | PHE | | 408 | −25.555 | 114.815 | −75.129 | 1.00 | 39.59 | |
| ATOM | 1916 | C | PHE | | 408 | −25.782 | 119.024 | −72.516 | 1.00 | 51.33 | |
| ATOM | 1917 | O | PHE | | 408 | −26.700 | 118.632 | −73.252 | 1.00 | 50.34 | |
| ATOM | 1918 | N | PHE | | 409 | −25.924 | 119.222 | −71.209 | 1.00 | 51.69 | |
| ATOM | 1919 | CA | PHE | | 409 | −27.159 | 118.928 | −70.498 | 1.00 | 53.34 | |
| ATOM | 1920 | CB | PHE | | 409 | −26.835 | 118.087 | −69.247 | 1.00 | 52.49 | |
| ATOM | 1921 | CG | PHE | | 409 | −26.514 | 116.636 | −69.536 | 1.00 | 49.84 | |
| ATOM | 1922 | CD1 | PHE | | 409 | −27.509 | 115.766 | −70.000 | 1.00 | 47.15 | |
| ATOM | 1923 | CD2 | PHE | | 409 | −25.215 | 116.138 | −69.336 | 1.00 | 46.48 | |
| ATOM | 1924 | CE1 | PHE | | 409 | −27.218 | 114.416 | −70.262 | 1.00 | 44.93 | |
| ATOM | 1925 | CE2 | PHE | | 409 | −24.920 | 114.808 | −69.593 | 1.00 | 43.99 | |
| ATOM | 1926 | CZ | PHE | | 409 | −25.927 | 113.944 | −70.058 | 1.00 | 43.30 | |
| ATOM | 1927 | C | PHE | | 409 | −28.007 | 120.138 | −70.076 | 1.00 | 55.22 | |
| ATOM | 1928 | O | PHE | | 409 | −28.365 | 120.275 | −68.896 | 1.00 | 55.90 | |
| ATOM | 1929 | N | LEU | | 410 | −28.330 | 121.018 | −71.018 | 1.00 | 56.10 | |
| ATOM | 1930 | CA | LEU | | 410 | −29.185 | 122.158 | −70.688 | 1.00 | 55.66 | |
| ATOM | 1931 | CB | LEU | | 410 | −28.881 | 123.355 | −71.598 | 1.00 | 56.88 | |
| ATOM | 1932 | CG | LEU | | 410 | −30.061 | 124.263 | −71.967 | 1.00 | 57.63 | |
| ATOM | 1933 | CD1 | LEU | | 410 | −29.522 | 125.592 | −72.450 | 1.00 | 58.73 | |
| ATOM | 1934 | CD2 | LEU | | 410 | −30.955 | 123.605 | −73.045 | 1.00 | 56.93 | |
| ATOM | 1935 | C | LEU | | 410 | −30.664 | 121.744 | −70.814 | 1.00 | 54.17 | |
| ATOM | 1936 | O | LEU | | 410 | −31.462 | 122.007 | −69.916 | 1.00 | 54.11 | |
| ATOM | 1937 | N | SER | | 411 | −31.016 | 121.081 | −71.910 | 1.00 | 52.16 | |
| ATOM | 1938 | CA | SER | | 411 | −32.389 | 120.640 | −72.150 | 1.00 | 52.53 | |
| ATOM | 1939 | CB | SER | | 411 | −32.495 | 120.044 | −73.548 | 1.00 | 53.52 | |
| ATOM | 1940 | OG | SER | | 411 | −31.890 | 118.751 | −73.607 | 1.00 | 50.84 | |
| ATOM | 1941 | C | SER | | 411 | −32.870 | 119.579 | −71.170 | 1.00 | 53.03 | |
| ATOM | 1942 | O | SER | | 411 | −33.795 | 118.811 | −71.468 | 1.00 | 52.87 | |
| ATOM | 1943 | N | ILE | | 412 | −32.297 | 119.550 | −69.981 | 1.00 | 53.34 | |
| ATOM | 1944 | CA | ILE | | 412 | −32.648 | 118.480 | −69.071 | 1.00 | 52.85 | |
| ATOM | 1945 | CB | ILE | | 412 | −31.334 | 117.739 | −68.701 | 1.00 | 52.94 | |
| ATOM | 1946 | CG2 | ILE | | 412 | −31.575 | 116.638 | −67.692 | 1.00 | 52.69 | |
| ATOM | 1947 | CG1 | ILE | | 412 | −30.728 | 117.154 | −69.988 | 1.00 | 52.08 | |
| ATOM | 1948 | CD1 | ILE | | 412 | −31.728 | 116.302 | −70.822 | 1.00 | 49.42 | |
| ATOM | 1949 | C | ILE | | 412 | −33.452 | 118.801 | −67.837 | 1.00 | 52.20 | |
| ATOM | 1950 | O | ILE | | 412 | −33.138 | 119.728 | −67.106 | 1.00 | 53.49 | |
| ATOM | 1951 | N | ASN | | 413 | −34.505 | 118.028 | −67.608 | 1.00 | 52.27 | |
| ATOM | 1952 | CA | ASN | | 413 | −35.329 | 118.214 | −66.420 | 1.00 | 52.73 | |
| ATOM | 1953 | CB | ASN | | 413 | −36.825 | 118.077 | −66.741 | 1.00 | 56.07 | |
| ATOM | 1954 | CG | ASN | | 413 | −37.698 | 118.919 | −65.801 | 1.00 | 59.17 | |
| ATOM | 1955 | OD1 | ASN | | 413 | −38.601 | 118.412 | −65.118 | 1.00 | 58.30 | |
| ATOM | 1956 | OD2 | ASN | | 413 | −37.416 | 120.225 | −65.764 | 1.00 | 60.58 | |
| ATOM | 1957 | C | ASN | | 413 | −34.926 | 117.114 | −65.461 | 1.00 | 51.48 | |
| ATOM | 1958 | O | ASN | | 413 | −35.598 | 116.080 | −65.369 | 1.00 | 49.23 | |
| ATOM | 1959 | N | TRP | | 414 | −33.827 | 117.329 | −64.749 | 1.00 | 51.21 | |
| ATOM | 1960 | CA | TRP | | 414 | −33.338 | 116.309 | −63.836 | 1.00 | 52.23 | |
| ATOM | 1961 | CB | TRP | | 414 | −32.142 | 116.843 | −63.040 | 1.00 | 52.76 | |
| ATOM | 1962 | CG | TRP | | 414 | −30.919 | 117.040 | −63.916 | 1.00 | 56.33 | |
| ATOM | 1963 | CD2 | TRP | | 414 | −30.173 | 116.019 | −64.622 | 1.00 | 57.44 | |
| ATOM | 1964 | CE2 | TRP | | 414 | −29.133 | 116.675 | −65.333 | 1.00 | 58.16 | |
| ATOM | 1965 | CE3 | TRP | | 414 | −30.274 | 114.622 | −64.710 | 1.00 | 57.73 | |
| ATOM | 1966 | CD1 | TRP | | 414 | −30.321 | 118.226 | −64.226 | 1.00 | 57.64 | |
| ATOM | 1967 | NE1 | TRP | | 414 | −29.249 | 118.015 | −65.080 | 1.00 | 58.59 | |
| ATOM | 1968 | CZ2 | TRP | | 414 | −28.208 | 115.981 | −66.134 | 1.00 | 57.67 | |
| ATOM | 1969 | CZ3 | TRP | | 414 | −29.349 | 113.930 | −65.510 | 1.00 | 57.83 | |
| ATOM | 1970 | CH2 | TRP | | 414 | −28.326 | 114.617 | −66.204 | 1.00 | 57.14 | |
| ATOM | 1971 | C | TRP | | 414 | −34.403 | 115.709 | −62.917 | 1.00 | 52.00 | |
| ATOM | 1972 | O | TRP | | 414 | −34.186 | 114.669 | −62.318 | 1.00 | 53.55 | |
| ATOM | 1973 | N | GLN | | 415 | −35.557 | 116.350 | −62.806 | 1.00 | 53.02 | |
| ATOM | 1974 | CA | GLN | | 415 | −36.628 | 115.808 | −61.979 | 1.00 | 52.35 | |
| ATOM | 1975 | CB | GLN | | 415 | −37.586 | 116.896 | −61.510 | 1.00 | 51.85 | |
| ATOM | 1976 | CG | GLN | | 415 | −37.357 | 117.413 | −60.128 | 1.00 | 50.98 | |
| ATOM | 1977 | CD | GLN | | 415 | −38.597 | 118.074 | −59.581 | 1.00 | 49.35 | |
| ATOM | 1978 | OE1 | GLN | | 415 | −39.702 | 117.610 | −59.829 | 1.00 | 49.48 | |
| ATOM | 1979 | NE2 | GLN | | 415 | −38.422 | 119.148 | −58.823 | 1.00 | 48.14 | |
| ATOM | 1980 | C | GLN | | 415 | −37.412 | 114.853 | −62.845 | 1.00 | 52.80 | |
| ATOM | 1981 | O | GLN | | 415 | −37.889 | 113.832 | −62.379 | 1.00 | 53.07 | |
| ATOM | 1982 | N | ASP | | 416 | −37.559 | 115.211 | −64.112 | 1.00 | 53.66 | |
| ATOM | 1983 | CA | ASP | | 416 | −38.293 | 114.387 | −65.055 | 1.00 | 56.63 | |
| ATOM | 1984 | CB | ASP | | 416 | −38.441 | 115.099 | −66.395 | 1.00 | 61.15 | |
| ATOM | 1985 | CG | ASP | | 416 | −39.645 | 116.005 | −66.436 | 1.00 | 64.26 | |
| ATOM | 1986 | OD1 | ASP | | 416 | −40.757 | 115.527 | −66.084 | 1.00 | 67.64 | |
| ATOM | 1987 | OD2 | ASP | | 416 | −39.472 | 117.180 | −66.828 | 1.00 | 64.63 | |
| ATOM | 1988 | C | ASP | | 416 | −37.597 | 113.072 | −65.274 | 1.00 | 55.71 | |
| ATOM | 1989 | O | ASP | | 416 | −38.245 | 112.058 | −65.503 | 1.00 | 54.51 | |
| ATOM | 1990 | N | VAL | | 417 | −36.268 | 113.114 | −65.228 | 1.00 | 55.85 | |
| ATOM | 1991 | CA | VAL | | 417 | −35.439 | 111.925 | −65.391 | 1.00 | 55.28 | |
| ATOM | 1992 | CB | VAL | | 417 | −33.936 | 112.238 | −65.172 | 1.00 | 55.40 | |
| ATOM | 1993 | CG1 | VAL | | 417 | −33.145 | 110.943 | −65.138 | 1.00 | 55.85 | |
| ATOM | 1994 | CG2 | VAL | | 417 | −33.409 | 113.162 | −66.273 | 1.00 | 53.47 | |
| ATOM | 1995 | C | VAL | | 417 | −35.843 | 110.879 | −64.359 | 1.00 | 55.08 | |
| ATOM | 1996 | O | VAL | | 417 | −36.481 | 109.884 | −64.691 | 1.00 | 54.28 | |
| ATOM | 1997 | N | VAL | | 418 | −35.499 | 111.124 | −63.099 | 1.00 | 54.29 | |
| ATOM | 1998 | CA | VAL | | 418 | −35.795 | 110.153 | −62.056 | 1.00 | 55.72 | |
| ATOM | 1999 | CB | VAL | | 418 | −35.348 | 110.662 | −60.674 | 1.00 | 55.31 | |
| ATOM | 2000 | CG1 | VAL | | 418 | −34.048 | 111.438 | −60.813 | 1.00 | 56.68 | |
| ATOM | 2001 | CG2 | VAL | | 418 | −36.416 | 111.528 | −60.064 | 1.00 | 56.29 | |
| ATOM | 2002 | C | VAL | | 418 | −37.257 | 109.767 | −61.997 | 1.00 | 56.54 | |
| ATOM | 2003 | O | VAL | | 418 | −37.622 | 108.728 | −61.420 | 1.00 | 55.83 | |
| ATOM | 2004 | N | GLN | | 419 | −38.094 | 110.600 | −62.603 | 1.00 | 58.05 | |
| ATOM | 2005 | CA | GLN | | 419 | −39.525 | 110.354 | −62.603 | 1.00 | 60.00 | |
| ATOM | 2006 | CB | GLN | | 419 | −40.273 | 111.691 | −62.624 | 1.00 | 60.59 | |
| ATOM | 2007 | CG | GLN | | 419 | −40.145 | 112.531 | −61.347 | 1.00 | 60.61 | |
| ATOM | 2008 | CD | GLN | | 419 | −41.070 | 112.054 | −60.251 | 1.00 | 61.81 | |
| ATOM | 2009 | OE1 | GLN | | 419 | −42.288 | 112.234 | −60.324 | 1.00 | 63.44 | |
| ATOM | 2010 | NE2 | GLN | | 419 | −40.503 | 111.430 | −59.233 | 1.00 | 61.86 | |
| ATOM | 2011 | C | GLN | | 419 | −39.967 | 109.489 | −63.781 | 1.00 | 61.10 | |
| ATOM | 2012 | O | GLN | | 419 | −41.143 | 109.155 | −63.893 | 1.00 | 61.80 | |
| ATOM | 2013 | N | LYS | | 420 | −39.015 | 109.101 | −64.633 | 1.00 | 62.64 | |
| ATOM | 2014 | CA | LYS | | 420 | −39.305 | 108.306 | −65.836 | 1.00 | 63.79 | |
| ATOM | 2015 | CB | LYS | | 420 | −39.859 | 106.919 | −65.475 | 1.00 | 63.30 | |
| ATOM | 2016 | CG | LYS | | 420 | −38.812 | 105.910 | −65.107 | 1.00 | 61.93 | |
| ATOM | 2017 | CD | LYS | | 420 | −39.356 | 104.487 | −65.106 | 1.00 | 61.18 | |
| ATOM | 2018 | CE | LYS | | 420 | −38.194 | 103.506 | −64.904 | 1.00 | 63.83 | |
| ATOM | 2019 | NZ | LYS | | 420 | −38.544 | 102.054 | −64.991 | 1.00 | 64.72 | |
| ATOM | 2020 | C | LYS | | 420 | −40.328 | 109.071 | −66.686 | 1.00 | 64.94 | |
| ATOM | 2021 | O | LYS | | 420 | −41.038 | 108.499 | −67.520 | 1.00 | 65.21 | |
| ATOM | 2022 | N | LYS | | 421 | −40.389 | 110.375 | −66.454 | 1.00 | 65.55 | |
| ATOM | 2023 | CA | LYS | | 421 | −41.305 | 111.243 | −67.157 | 1.00 | 66.82 | |
| ATOM | 2024 | CB | LYS | | 421 | −41.375 | 112.602 | −66.448 | 1.00 | 69.10 | |
| ATOM | 2025 | CG | LYS | | 421 | −42.722 | 112.866 | −65.761 | 1.00 | 71.17 | |
| ATOM | 2026 | CD | LYS | | 421 | −43.160 | 111.712 | −64.841 | 1.00 | 73.41 | |
| ATOM | 2027 | CE | LYS | | 421 | −44.649 | 111.810 | −64.441 | 1.00 | 74.24 | |
| ATOM | 2028 | NZ | LYS | | 421 | −45.029 | 113.065 | −63.704 | 1.00 | 74.43 | |
| ATOM | 2029 | C | LYS | | 421 | −40.880 | 111.405 | −68.606 | 1.00 | 67.07 | |
| ATOM | 2030 | O | LYS | | 421 | −41.719 | 111.535 | −69.493 | 1.00 | 67.37 | |
| ATOM | 2031 | N | LEU | | 422 | −39.576 | 111.389 | −68.850 | 1.00 | 66.80 | |
| ATOM | 2032 | CA | LEU | | 422 | −39.073 | 111.511 | −70.215 | 1.00 | 66.69 | |
| ATOM | 2033 | CB | LEU | | 422 | −37.543 | 111.507 | −70.230 | 1.00 | 66.44 | |
| ATOM | 2034 | CG | LEU | | 422 | −36.956 | 112.734 | −69.534 | 1.00 | 67.01 | |
| ATOM | 2035 | CD1 | LEU | | 422 | −35.499 | 112.889 | −69.926 | 1.00 | 66.42 | |
| ATOM | 2036 | CD2 | LEU | | 422 | −37.751 | 113.982 | −69.938 | 1.00 | 66.61 | |
| ATOM | 2037 | C | LEU | | 422 | −39.593 | 110.390 | −71.107 | 1.00 | 66.24 | |
| ATOM | 2038 | O | LEU | | 422 | −40.064 | 109.359 | −70.635 | 1.00 | 65.39 | |
| ATOM | 2039 | N | LEU | | 423 | −39.515 | 110.612 | −72.409 | 1.00 | 66.58 | |
| ATOM | 2040 | CA | LEU | | 423 | −39.976 | 109.628 | −73.364 | 1.00 | 66.62 | |
| ATOM | 2041 | CB | LEU | | 423 | −40.843 | 110.306 | −74.438 | 1.00 | 66.42 | |
| ATOM | 2042 | CG | LEU | | 423 | −41.560 | 109.422 | −75.476 | 1.00 | 68.09 | |
| ATOM | 2043 | CD1 | LEU | | 423 | −42.789 | 108.747 | −74.838 | 1.00 | 67.26 | |
| ATOM | 2044 | CD2 | LEU | | 423 | −41.979 | 110.258 | −76.683 | 1.00 | 66.82 | |
| ATOM | 2045 | C | LEU | | 423 | −38.755 | 108.959 | −74.002 | 1.00 | 66.65 | |
| ATOM | 2046 | O | LEU | | 423 | −37.840 | 109.629 | −74.500 | 1.00 | 66.10 | |
| ATOM | 2047 | N | PRO | | 424 | −38.708 | 107.621 | −73.959 | 1.00 | 66.46 | |
| ATOM | 2048 | CD | PRO | | 424 | −39.698 | 106.650 | −73.455 | 1.00 | 66.23 | |
| ATOM | 2049 | CA | PRO | | 424 | −37.563 | 106.943 | −74.564 | 1.00 | 66.89 | |
| ATOM | 2050 | CB | PRO | | 424 | −37.826 | 105.477 | −74.232 | 1.00 | 66.75 | |
| ATOM | 2051 | CG | PRO | | 424 | −39.334 | 105.406 | −74.205 | 1.00 | 66.65 | |
| ATOM | 2052 | C | PRO | | 424 | −37.644 | 107.231 | −76.062 | 1.00 | 67.60 | |
| ATOM | 2053 | O | PRO | | 424 | −38.714 | 107.079 | −76.671 | 1.00 | 67.91 | |
| ATOM | 2054 | N | PRO | | 425 | −36.531 | 107.680 | −76.670 | 1.00 | 67.38 | |
| ATOM | 2055 | CD | PRO | | 425 | −35.258 | 108.039 | −76.023 | 1.00 | 67.26 | |
| ATOM | 2056 | CA | PRO | | 425 | −36.498 | 107.990 | −78.106 | 1.00 | 66.27 | |
| ATOM | 2057 | CB | PRO | | 425 | −35.223 | 108.806 | −78.254 | 1.00 | 66.59 | |
| ATOM | 2058 | CG | PRO | | 425 | −34.336 | 108.213 | −77.214 | 1.00 | 67.16 | |
| ATOM | 2059 | C | PRO | | 425 | −36.503 | 106.745 | −78.980 | 1.00 | 66.14 | |
| ATOM | 2060 | O | PRO | | 425 | −36.358 | 106.826 | −80.202 | 1.00 | 65.38 | |
| ATOM | 2061 | N | PHE | | 426 | −36.671 | 105.595 | −78.339 | 1.00 | 65.30 | |
| ATOM | 2062 | CA | PHE | | 426 | −36.726 | 104.317 | −79.027 | 1.00 | 65.63 | |
| ATOM | 2063 | CB | PHE | | 426 | −35.322 | 103.782 | −79.325 | 1.00 | 65.22 | |
| ATOM | 2064 | CG | PHE | | 426 | −35.297 | 102.322 | −79.685 | 1.00 | 63.93 | |
| ATOM | 2065 | CD1 | PHE | | 426 | −35.632 | 101.901 | −80.963 | 1.00 | 63.23 | |
| ATOM | 2066 | CD2 | PHE | | 426 | −35.017 | 101.358 | −78.716 | 1.00 | 65.40 | |
| ATOM | 2067 | CE1 | PHE | | 426 | −35.699 | 100.550 | −81.274 | 1.00 | 62.86 | |
| ATOM | 2068 | CE2 | PHE | | 426 | −35.082 | 99.996 | −79.017 | 1.00 | 64.40 | |
| ATOM | 2069 | CZ | PHE | | 426 | −35.425 | 99.597 | −80.299 | 1.00 | 63.95 | |
| ATOM | 2070 | C | PHE | | 426 | −37.422 | 103.354 | −78.101 | 1.00 | 66.77 | |
| ATOM | 2071 | O | PHE | | 426 | −36.981 | 103.141 | −76.974 | 1.00 | 67.75 | |
| ATOM | 2072 | N | LYS | | 427 | −38.517 | 102.773 | −78.553 | 1.00 | 68.26 | |
| ATOM | 2073 | CA | LYS | | 427 | −39.184 | 101.826 | −77.698 | 1.00 | 71.07 | |
| ATOM | 2074 | CB | LYS | | 427 | −40.684 | 102.075 | −77.609 | 1.00 | 73.34 | |
| ATOM | 2075 | CG | LYS | | 427 | −41.421 | 100.969 | −76.845 | 1.00 | 76.46 | |
| ATOM | 2076 | CD | LYS | | 427 | −42.730 | 101.483 | −76.267 | 1.00 | 79.30 | |
| ATOM | 2077 | CE | LYS | | 427 | −43.315 | 100.524 | −75.244 | 1.00 | 80.07 | |
| ATOM | 2078 | NZ | LYS | | 427 | −44.509 | 101.121 | −74.569 | 1.00 | 81.44 | |
| ATOM | 2079 | C | LYS | | 427 | −38.963 | 100.438 | −78.214 | 1.00 | 72.16 | |
| ATOM | 2080 | O | LYS | | 427 | −39.269 | 100.141 | −79.362 | 1.00 | 72.45 | |
| ATOM | 2081 | N | PRO | | 428 | −38.412 | 99.566 | −77.364 | 1.00 | 73.23 | |
| ATOM | 2082 | CD | PRO | | 428 | −37.908 | 99.901 | −76.024 | 1.00 | 72.33 | |
| ATOM | 2083 | CA | PRO | | 428 | −38.129 | 98.168 | −77.687 | 1.00 | 74.21 | |
| ATOM | 2084 | CB | PRO | | 428 | −37.604 | 97.632 | −76.370 | 1.00 | 73.62 | |
| ATOM | 2085 | CG | PRO | | 428 | −36.904 | 98.823 | −75.801 | 1.00 | 72.83 | |
| ATOM | 2086 | C | PRO | | 428 | −39.448 | 97.530 | −78.096 | 1.00 | 75.76 | |
| ATOM | 2087 | O | PRO | | 428 | −40.505 | 98.013 | −77.695 | 1.00 | 76.36 | |
| ATOM | 2088 | N | GLN | | 429 | −39.400 | 96.457 | −78.879 | 1.00 | 77.72 | |
| ATOM | 2089 | CA | GLN | | 429 | −40.633 | 95.824 | −79.338 | 1.00 | 80.67 | |
| ATOM | 2090 | CB | GLN | | 429 | −40.477 | 95.347 | −80.795 | 1.00 | 81.80 | |
| ATOM | 2091 | CG | GLN | | 429 | −40.279 | 96.476 | −81.816 | 1.00 | 83.38 | |
| ATOM | 2092 | CD | GLN | | 429 | −38.905 | 96.442 | −82.489 | 1.00 | 85.42 | |
| ATOM | 2093 | OE1 | GLN | | 429 | −37.916 | 96.005 | −81.891 | 1.00 | 87.30 | |
| ATOM | 2094 | NE2 | GLN | | 429 | −38.837 | 96.926 | −83.731 | 1.00 | 84.87 | |
| ATOM | 2095 | C | GLN | | 429 | −41.118 | 94.669 | −78.476 | 1.00 | 81.74 | |
| ATOM | 2096 | O | GLN | | 429 | −40.983 | 93.511 | −78.865 | 1.00 | 82.91 | |
| ATOM | 2097 | N | VAL | | 430 | −41.702 | 94.976 | −77.320 | 1.00 | 82.78 | |
| ATOM | 2098 | CA | VAL | | 430 | −42.204 | 93.932 | −76.420 | 1.00 | 84.69 | |
| ATOM | 2099 | CB | VAL | | 430 | −41.168 | 93.618 | −75.284 | 1.00 | 84.62 | |
| ATOM | 2100 | CG1 | VAL | | 430 | −39.900 | 93.018 | −75.877 | 1.00 | 84.85 | |
| ATOM | 2101 | CG2 | VAL | | 430 | −40.822 | 94.880 | −74.515 | 1.00 | 84.52 | |
| ATOM | 2102 | C | VAL | | 430 | −43.566 | 94.258 | −75.773 | 1.00 | 85.71 | |
| ATOM | 2103 | O | VAL | | 430 | −43.749 | 95.333 | −75.188 | 1.00 | 86.15 | |
| ATOM | 2104 | N | THR | | 431 | −44.512 | 93.321 | −75.884 | 1.00 | 86.28 | |
| ATOM | 2105 | CA | THR | | 431 | −45.862 | 93.477 | −75.314 | 1.00 | 86.70 | |
| ATOM | 2106 | CB | THR | | 431 | −46.700 | 92.163 | −75.454 | 1.00 | 86.82 | |
| ATOM | 2107 | OG1 | THR | | 431 | −46.569 | 91.632 | −76.779 | 1.00 | 86.80 | |
| ATOM | 2108 | CG2 | THR | | 431 | −48.171 | 92.432 | −75.169 | 1.00 | 85.55 | |
| ATOM | 2109 | C | THR | | 431 | −45.738 | 93.775 | −73.819 | 1.00 | 86.81 | |
| ATOM | 2110 | O | THR | | 431 | −45.720 | 94.927 | −73.381 | 1.00 | 85.92 | |
| ATOM | 2111 | N | SER | | 432 | −45.664 | 92.694 | −73.052 | 1.00 | 88.08 | |
| ATOM | 2112 | CA | SER | | 432 | −45.515 | 92.743 | −71.606 | 1.00 | 89.37 | |
| ATOM | 2113 | CB | SER | | 432 | −46.410 | 91.683 | −70.940 | 1.00 | 89.59 | |
| ATOM | 2114 | OG | SER | | 432 | −46.114 | 90.373 | −71.413 | 1.00 | 88.93 | |
| ATOM | 2115 | C | SER | | 432 | −44.051 | 92.433 | −71.300 | 1.00 | 90.26 | |
| ATOM | 2116 | O | SER | | 432 | −43.324 | 91.917 | −72.161 | 1.00 | 90.49 | |
| ATOM | 2117 | N | GLU | | 433 | −43.623 | 92.752 | −70.082 | 1.00 | 90.53 | |
| ATOM | 2118 | CA | GLU | | 433 | −42.253 | 92.483 | −69.661 | 1.00 | 90.91 | |
| ATOM | 2119 | CB | GLU | | 433 | −42.077 | 92.887 | −68.197 | 1.00 | 90.87 | |
| ATOM | 2120 | CG | GLU | | 433 | −43.355 | 92.807 | −67.388 | 1.00 | 91.01 | |
| ATOM | 2121 | CD | GLU | | 433 | −43.538 | 94.017 | −66.483 | 1.00 | 91.12 | |
| ATOM | 2122 | OE1 | GLU | | 433 | −42.690 | 94.220 | −65.584 | 1.00 | 90.23 | |
| ATOM | 2123 | OE2 | GLU | | 433 | −44.526 | 94.767 | −66.675 | 1.00 | 90.80 | |
| ATOM | 2124 | C | GLU | | 433 | −41.963 | 90.995 | −69.859 | 1.00 | 90.98 | |
| ATOM | 2125 | O | GLU | | 433 | −40.809 | 90.580 | −70.026 | 1.00 | 90.47 | |
| ATOM | 2126 | N | VAL | | 434 | −43.035 | 90.206 | −69.859 | 1.00 | 90.73 | |
| ATOM | 2127 | CA | VAL | | 434 | −42.950 | 88.769 | −70.049 | 1.00 | 89.79 | |
| ATOM | 2128 | CB | VAL | | 434 | −44.265 | 88.093 | −69.629 | 1.00 | 90.32 | |
| ATOM | 2129 | CG1 | VAL | | 434 | −44.234 | 86.614 | −69.958 | 1.00 | 89.56 | |
| ATOM | 2130 | CG2 | VAL | | 434 | −44.472 | 88.288 | −68.141 | 1.00 | 91.06 | |
| ATOM | 2131 | C | VAL | | 434 | −42.625 | 88.419 | −71.496 | 1.00 | 89.19 | |
| ATOM | 2132 | O | VAL | | 434 | −42.378 | 87.258 | −71.812 | 1.00 | 89.05 | |
| ATOM | 2133 | N | ASP | | 435 | −42.618 | 89.411 | −72.381 | 0.00 | 89.50 | |
| ATOM | 2134 | CA | ASP | | 435 | −42.287 | 89.119 | −73.767 | 1.00 | 89.74 | |
| ATOM | 2135 | CB | ASP | | 435 | −42.537 | 90.319 | −74.683 | 1.00 | 89.77 | |
| ATOM | 2136 | CG | ASP | | 435 | −42.274 | 89.985 | −76.145 | 1.00 | 90.31 | |
| ATOM | 2137 | OD1 | ASP | | 435 | −42.890 | 89.020 | −76.641 | 1.00 | 90.76 | |
| ATOM | 2138 | OD2 | ASP | | 435 | −41.453 | 90.667 | −76.796 | 1.00 | 89.77 | |
| ATOM | 2139 | C | ASP | | 435 | −40.813 | 88.728 | −73.802 | 1.00 | 89.83 | |
| ATOM | 2140 | O | ASP | | 435 | −39.933 | 89.522 | −73.471 | 1.00 | 89.50 | |
| ATOM | 2141 | N | THR | | 436 | −40.551 | 87.495 | −74.213 | 1.00 | 90.03 | |
| ATOM | 2142 | CA | THR | | 436 | −39.193 | 86.974 | −74.245 | 1.00 | 90.44 | |
| ATOM | 2143 | CB | THR | | 436 | −39.164 | 85.530 | −73.725 | 1.00 | 89.61 | |
| ATOM | 2144 | OG1 | THR | | 436 | −39.768 | 84.674 | −74.702 | 1.00 | 88.62 | |
| ATOM | 2145 | CG2 | THR | | 436 | −39.925 | 85.417 | −72.404 | 1.00 | 88.03 | |
| ATOM | 2146 | C | THR | | 436 | −38.552 | 86.964 | −75.624 | 1.00 | 91.23 | |
| ATOM | 2147 | O | THR | | 436 | −37.872 | 86.002 | −75.981 | 1.00 | 91.04 | |
| ATOM | 2148 | N | ARG | | 437 | −38.742 | 88.019 | −76.403 | 1.00 | 92.58 | |
| ATOM | 2149 | CA | ARG | | 437 | −38.148 | 88.006 | −77.727 | 1.00 | 94.08 | |
| ATOM | 2150 | CB | ARG | | 437 | −39.012 | 88.785 | −78.732 | 1.00 | 95.34 | |
| ATOM | 2151 | CG | ARG | | 437 | −38.907 | 90.293 | −78.701 | 1.00 | 97.36 | |
| ATOM | 2152 | CD | ARG | | 437 | −39.504 | 90.871 | −79.993 | 1.00 | 99.69 | |
| ATOM | 2153 | NE | ARG | | 437 | −40.923 | 90.533 | −80.159 | 1.00 | 101.02 | |
| ATOM | 2154 | CZ | ARG | | 437 | −41.573 | 90.556 | −81.321 | 1.00 | 100.67 | |
| ATOM | 2155 | NH1 | ARG | | 437 | −40.941 | 90.899 | −82.437 | 1.00 | 100.49 | |
| ATOM | 2156 | NH2 | ARG | | 437 | −42.858 | 90.229 | −81.369 | 1.00 | 100.57 | |
| ATOM | 2157 | C | ARG | | 437 | −36.708 | 88.499 | −77.742 | 1.00 | 93.94 | |
| ATOM | 2158 | O | ARG | | 437 | −35.910 | 88.050 | −78.563 | 1.00 | 94.51 | |
| ATOM | 2159 | N | TYR | | 438 | −36.360 | 89.405 | −76.835 | 1.00 | 93.65 | |
| ATOM | 2160 | CA | TYR | | 438 | −34.985 | 89.893 | −76.788 | 1.00 | 93.41 | |
| ATOM | 2161 | CB | TYR | | 438 | −34.900 | 91.280 | −76.147 | 1.00 | 92.61 | |
| ATOM | 2162 | CG | TYR | | 438 | −35.288 | 92.417 | −77.061 | 1.00 | 92.18 | |
| ATOM | 2163 | CD1 | TYR | | 438 | −36.580 | 92.944 | −77.046 | 1.00 | 91.98 | |
| ATOM | 2164 | CE1 | TYR | | 438 | −36.945 | 93.978 | −77.903 | 1.00 | 91.83 | |
| ATOM | 2165 | CD2 | TYR | | 438 | −34.365 | 92.957 | −77.960 | 1.00 | 91.59 | |
| ATOM | 2166 | CE2 | TYR | | 438 | −34.717 | 93.992 | −78.821 | 1.00 | 90.95 | |
| ATOM | 2167 | CZ | TYR | | 438 | −36.009 | 94.496 | −78.790 | 1.00 | 91.35 | |
| ATOM | 2168 | OH | TYR | | 438 | −36.370 | 95.505 | −79.657 | 1.00 | 91.56 | |
| ATOM | 2169 | C | TYR | | 438 | −34.116 | 88.937 | −75.988 | 1.00 | 93.68 | |
| ATOM | 2170 | O | TYR | | 438 | −33.101 | 89.355 | −75.421 | 1.00 | 94.37 | |
| ATOM | 2171 | N | PHE | | 439 | −34.516 | 87.664 | −75.935 | 1.00 | 93.70 | |
| ATOM | 2172 | CA | PHE | | 439 | −33.765 | 86.654 | −75.190 | 1.00 | 93.63 | |
| ATOM | 2173 | CB | PHE | | 439 | −34.698 | 85.891 | −74.232 | 1.00 | 92.31 | |
| ATOM | 2174 | CG | PHE | | 439 | −35.246 | 86.736 | −73.096 | 1.00 | 91.84 | |
| ATOM | 2175 | CD1 | PHE | | 439 | −36.241 | 86.239 | −72.255 | 1.00 | 91.77 | |
| ATOM | 2176 | CD2 | PHE | | 439 | −34.788 | 88.039 | −72.882 | 1.00 | 91.11 | |
| ATOM | 2177 | CE1 | PHE | | 439 | −36.777 | 87.028 | −71.213 | 1.00 | 91.16 | |
| ATOM | 2178 | CE2 | PHE | | 439 | −35.314 | 88.833 | −71.848 | 1.00 | 90.75 | |
| ATOM | 2179 | CZ | PHE | | 439 | −36.313 | 88.325 | −71.015 | 1.00 | 90.31 | |
| ATOM | 2180 | C | PHE | | 439 | −33.006 | 85.671 | −76.082 | 1.00 | 94.41 | |
| ATOM | 2181 | O | PHE | | 439 | −32.915 | 84.488 | −75.778 | 1.00 | 95.17 | |
| ATOM | 2182 | N | ASP | | 440 | −32.466 | 86.178 | −77.185 | 1.00 | 95.67 | |
| ATOM | 2183 | CA | ASP | | 440 | −31.671 | 85.394 | −78.131 | 1.00 | 97.43 | |
| ATOM | 2184 | CB | ASP | | 440 | −32.530 | 84.911 | −79.297 | 1.00 | 98.16 | |
| ATOM | 2185 | CG | ASP | | 440 | −33.751 | 84.140 | −78.841 | 1.00 | 98.95 | |
| ATOM | 2186 | OD1 | ASP | | 440 | −34.674 | 84.766 | −78.269 | 1.00 | 98.96 | |
| ATOM | 2187 | OD2 | ASP | | 440 | −33.785 | 82.906 | −79.051 | 1.00 | 99.24 | |
| ATOM | 2188 | C | ASP | | 440 | −30.626 | 86.387 | −78.635 | 1.00 | 98.95 | |
| ATOM | 2189 | O | ASP | | 440 | −30.140 | 87.207 | −77.853 | 1.00 | 99.32 | |
| ATOM | 2190 | N | ASP | | 441 | −30.280 | 86.337 | −79.922 | 1.00 | 99.92 | |
| ATOM | 2191 | CA | ASP | | 441 | −29.299 | 87.289 | −80.465 | 1.00 | 100.41 | |
| ATOM | 2192 | CB | ASP | | 441 | −28.272 | 86.586 | −81.370 | 1.00 | 100.96 | |
| ATOM | 2193 | CG | ASP | | 441 | −27.259 | 85.755 | −80.590 | 1.00 | 101.62 | |
| ATOM | 2194 | OD1 | ASP | | 441 | −27.650 | 84.708 | −80.020 | 1.00 | 102.78 | |
| ATOM | 2195 | OD2 | ASP | | 441 | −26.070 | 86.155 | −80.553 | 1.00 | 101.62 | |
| ATOM | 2196 | C | ASP | | 441 | −29.979 | 88.403 | −81.267 | 1.00 | 99.97 | |
| ATOM | 2197 | O | ASP | | 441 | −29.457 | 88.722 | −82.363 | 1.00 | 100.19 | |
| ATOM | 2198 | OXT | ASP | | 441 | −31.006 | 88.949 | −80.788 | 1.00 | 99.08 | |
| ATOM | 2199 | OH2 | TIP | S | 1 | −28.279 | 117.130 | −75.119 | 1.00 | 34.27 | S |
| ATOM | 2200 | OH2 | TIP | S | 2 | −20.338 | 90.232 | −73.418 | 1.00 | 37.56 | S |
| ATOM | 2201 | OH2 | TIP | S | 3 | −40.040 | 69.855 | −59.741 | 1.00 | 51.84 | S |
| ATOM | 2202 | OH2 | TIP | S | 4 | −25.225 | 75.462 | −53.383 | 1.00 | 92.94 | S |
| ATOM | 2203 | OH2 | TIP | S | 5 | −10.577 | 83.823 | −50.768 | 1.00 | 52.45 | S |
| ATOM | 2204 | OH2 | TIP | S | 6 | −20.050 | 83.628 | −46.136 | 1.00 | 40.14 | S |
| ATOM | 2205 | OH2 | TIP | S | 8 | −25.025 | 95.943 | −72.991 | 1.00 | 54.25 | S |
| ATOM | 2206 | OH2 | TIP | S | 9 | −16.147 | 93.778 | −51.712 | 1.00 | 57.82 | S |
| ATOM | 2207 | OH2 | TIP | S | 10 | −14.459 | 99.445 | −70.266 | 1.00 | 55.95 | S |
| ATOM | 2208 | OH2 | TIP | S | 11 | −29.240 | 120.386 | −73.296 | 1.00 | 60.45 | S |
| ATOM | 2209 | OH2 | TIP | S | 12 | −17.965 | 101.307 | −71.613 | 1.00 | 46.85 | S |
| ATOM | 2210 | OH2 | TIP | S | 13 | −15.267 | 108.531 | −72.968 | 1.00 | 91.70 | S |
| ATOM | 2211 | OH2 | TIP | S | 14 | −35.604 | 84.032 | −67.027 | 1.00 | 80.01 | S |
| ATOM | 2212 | OH2 | TIP | S | 16 | −24.325 | 112.109 | −55.117 | 1.00 | 48.28 | S |
| ATOM | 2213 | OH2 | TIP | S | 17 | −19.694 | 85.685 | −75.442 | 1.00 | 49.20 | S |
| ATOM | 2214 | OH2 | TIP | S | 18 | −14.471 | 111.840 | −92.040 | 1.00 | 66.51 | S |
| ATOM | 2215 | OH2 | TIP | S | 19 | −29.978 | 105.957 | −69.758 | 1.00 | 65.82 | S |
| ATOM | 2216 | OH2 | TIP | S | 21 | −34.418 | 119.302 | −75.361 | 1.00 | 77.64 | S |
| ATOM | 2217 | OH2 | TIP | S | 22 | −38.864 | 101.588 | −60.192 | 1.00 | 58.22 | S |
| ATOM | 2218 | OH2 | TIP | S | 23 | −24.772 | 100.358 | −49.592 | 1.00 | 64.47 | S |
| ATOM | 2219 | OH2 | TIP | S | 25 | −17.774 | 102.854 | −63.371 | 1.00 | 40.15 | S |
| ATOM | 2220 | OH2 | TIP | S | 26 | −25.095 | 110.786 | −74.186 | 1.00 | 77.99 | S |
| ATOM | 2221 | OH2 | TIP | S | 27 | −6.339 | 113.185 | −80.728 | 1.00 | 75.97 | S |
| ATOM | 2222 | OH2 | TIP | S | 28 | 2.253 | 106.824 | −60.404 | 1.00 | 62.87 | S |
| ATOM | 2223 | OH2 | TIP | S | 29 | −21.066 | 90.649 | −77.276 | 1.00 | 54.35 | S |
| ATOM | 2224 | OH2 | TIP | S | 30 | −25.931 | 91.782 | −59.896 | 1.00 | 79.95 | S |
| ATOM | 2225 | OH2 | TIP | S | 31 | −38.876 | 102.793 | −82.070 | 1.00 | 35.31 | S |
| ATOM | 2226 | OH2 | TIP | S | 32 | −28.265 | 121.697 | −61.510 | 1.00 | 73.14 | S |
| ATOM | 2227 | OH2 | TIP | S | 33 | −17.629 | 102.276 | −52.451 | 1.00 | 50.42 | S |
| ATOM | 2228 | OH2 | TIP | S | 36 | −19.186 | 69.946 | −50.434 | 1.00 | 55.26 | S |
| ATOM | 2229 | OH2 | TIP | S | 37 | −34.577 | 104.057 | −75.386 | 1.00 | 59.33 | S |
| ATOM | 2230 | OH2 | TIP | S | 39 | −43.307 | 97.845 | −75.637 | 1.00 | 84.67 | S |
| ATOM | 2231 | OH2 | TIP | S | 40 | −38.231 | 91.930 | −61.441 | 1.00 | 67.57 | S |
| ATOM | 2232 | OH2 | TIP | S | 42 | −13.260 | 92.477 | −62.358 | 1.00 | 47.45 | S |
| ATOM | 2233 | OH2 | TIP | S | 43 | −30.252 | 120.215 | −67.130 | 1.00 | 63.27 | S |
| ATOM | 2234 | OH2 | TIP | S | 45 | −12.970 | 112.336 | −88.089 | 1.00 | 61.99 | S |
| ATOM | 2235 | OH2 | TIP | S | 46 | −3.233 | 95.832 | −73.294 | 1.00 | 70.30 | S |
| ATOM | 2236 | OH2 | TIP | S | 47 | −33.354 | 91.917 | −73.019 | 1.00 | 50.21 | S |
| ATOM | 2237 | OH2 | TIP | S | 48 | −47.678 | 112.329 | −66.053 | 1.00 | 58.36 | S |
| ATOM | 2238 | OH2 | TIP | S | 49 | −18.930 | 76.549 | −47.815 | 1.00 | 36.97 | S |
| ATOM | 2239 | OH2 | TIP | S | 50 | −17.946 | 91.866 | −77.524 | 1.00 | 67.78 | S |
| ATOM | 2240 | OH2 | TIP | S | 51 | −15.059 | 118.345 | −90.201 | 1.00 | 68.42 | S |
| ATOM | 2241 | OH2 | TIP | S | 52 | −30.824 | 114.259 | −74.515 | 1.00 | 91.39 | S |
| ATOM | 2242 | OH2 | TIP | S | 53 | −31.532 | 108.464 | −82.347 | 1.00 | 61.82 | S |
| ATOM | 2243 | OH2 | TIP | S | 54 | −37.057 | 103.213 | −57.306 | 1.00 | 55.29 | S |
| ATOM | 2244 | OH2 | TIP | S | 55 | −15.159 | 89.223 | −47.068 | 1.00 | 62.08 | S |
| ATOM | 2245 | OH2 | TIP | S | 56 | −15.327 | 96.363 | −53.985 | 1.00 | 63.66 | S |
| ATOM | 2246 | OH2 | TIP | S | 57 | −8.763 | 96.325 | −78.630 | 1.00 | 68.63 | S |
| ATOM | 2247 | OH2 | TIP | S | 58 | −3.897 | 124.612 | −68.352 | 1.00 | 54.99 | S |
| ATOM | 2248 | OH2 | TIP | S | 59 | −21.553 | 74.980 | −60.637 | 1.00 | 66.26 | S |
| ATOM | 2249 | OH2 | TIP | S | 60 | −16.332 | 117.985 | −81.778 | 1.00 | 82.09 | S |
| ATOM | 2250 | OH2 | TIP | S | 62 | −10.541 | 92.931 | −60.120 | 1.00 | 61.28 | S |
| ATOM | 2251 | OH2 | TIP | S | 63 | −19.368 | 110.784 | −52.826 | 1.00 | 57.63 | S |
| ATOM | 2252 | OH2 | TIP | S | 65 | −2.246 | 124.598 | −64.545 | 1.00 | 61.06 | S |
| ATOM | 2253 | OH2 | TIP | S | 66 | −19.885 | 95.377 | −47.136 | 1.00 | 94.88 | S |
| ATOM | 2254 | OH2 | TIP | S | 67 | −33.235 | 105.665 | −77.851 | 1.00 | 93.94 | S |
| ATOM | 2255 | OH2 | TIP | S | 68 | −16.225 | 88.770 | −80.454 | 1.00 | 52.18 | S |
| ATOM | 2256 | OH2 | TIP | S | 69 | −22.493 | 124.406 | −77.955 | 1.00 | 77.26 | S |
| ATOM | 2257 | OH2 | TIP | S | 70 | −45.802 | 113.820 | −68.412 | 1.00 | 56.85 | S |
| ATOM | 2258 | OH2 | TIP | S | 73 | −29.372 | 123.275 | −68.017 | 1.00 | 77.12 | S |
| ATOM | 2259 | OH2 | TIP | S | 74 | −20.194 | 110.647 | −75.811 | 1.00 | 72.87 | S |
| ATOM | 2260 | OH2 | TIP | S | 75 | −13.474 | 110.285 | −57.377 | 1.00 | 52.18 | S |
| ATOM | 2261 | OH2 | TIP | S | 76 | −18.889 | 122.892 | −58.058 | 1.00 | 63.85 | S |
| ATOM | 2262 | OH2 | TIP | S | 77 | −33.242 | 114.790 | −69.241 | 1.00 | 90.71 | S |
| ATOM | 2263 | OH2 | TIP | S | 78 | −37.240 | 99.603 | −86.817 | 1.00 | 65.07 | S |
| ATOM | 2264 | OH2 | TIP | S | 79 | −42.916 | 107.508 | −66.443 | 1.00 | 71.85 | S |
| ATOM | 2265 | OH2 | TIP | S | 80 | −12.507 | 89.539 | −53.003 | 1.00 | 52.98 | S |
| ATOM | 2266 | OH2 | TIP | S | 81 | −22.124 | 125.331 | −65.900 | 1.00 | 37.55 | S |
| ATOM | 2267 | OH2 | TIP | S | 82 | −32.136 | 110.814 | −81.456 | 1.00 | 93.66 | S |
| ATOM | 2268 | OH2 | TIP | S | 83 | −34.216 | 117.264 | −58.785 | 1.00 | 49.62 | S |
| ATOM | 2269 | OH2 | TIP | S | 84 | −22.066 | 110.808 | −48.305 | 1.00 | 68.96 | S |
| ATOM | 2270 | OH2 | TIP | S | 85 | 1.915 | 96.956 | −78.632 | 1.00 | 75.10 | S |
| ATOM | 2271 | OH2 | TIP | S | 86 | −6.991 | 109.235 | −79.459 | 1.00 | 57.41 | S |
| ATOM | 2272 | OH2 | TIP | S | 87 | −22.550 | 116.451 | −54.448 | 1.00 | 63.30 | S |
| ATOM | 2273 | OH2 | TIP | S | 88 | −47.364 | 94.770 | −65.585 | 1.00 | 50.84 | S |
| ATOM | 2274 | OH2 | TIP | S | 89 | −18.529 | 102.462 | −73.721 | 1.00 | 67.48 | S |
| ATOM | 2275 | OH2 | TIP | S | 90 | −40.581 | 100.708 | −87.003 | 1.00 | 63.83 | S |
| ATOM | 2276 | OH2 | TIP | S | 91 | −44.190 | 104.278 | −63.609 | 1.00 | 68.23 | S |
| ATOM | 2277 | OH2 | TIP | S | 93 | −39.737 | 97.108 | −52.484 | 1.00 | 62.42 | S |
| ATOM | 2278 | OH2 | TIP | S | 95 | −18.784 | 124.281 | −72.546 | 1.00 | 69.88 | S |
| ATOM | 2279 | OH2 | TIP | S | 96 | −22.323 | 77.228 | −65.503 | 1.00 | 81.91 | S |
| ATOM | 2280 | OH2 | TIP | S | 97 | −41.947 | 112.253 | −72.797 | 1.00 | 80.67 | S |
| ATOM | 2281 | OH2 | TIP | S | 98 | −30.279 | 91.655 | −64.299 | 1.00 | 61.84 | S |
| ATOM | 2282 | OH2 | TIP | S | 99 | −10.995 | 81.597 | −40.763 | 1.00 | 56.49 | S |
| ATOM | 2283 | OH2 | TIP | S | 101 | −30.653 | 85.509 | −82.750 | 1.00 | 56.57 | S |
| ATOM | 2284 | OH2 | TIP | S | 102 | −31.914 | 113.319 | −61.166 | 1.00 | 72.30 | S |
| ATOM | 2285 | OH2 | TIP | S | 103 | −6.815 | 129.917 | −62.463 | 1.00 | 51.33 | S |
| ATOM | 2286 | OH2 | TIP | S | 104 | −15.562 | 102.708 | −65.118 | 1.00 | 42.22 | S |
| ATOM | 2287 | OH2 | TIP | S | 105 | −46.346 | 107.948 | −63.277 | 1.00 | 69.86 | S |
| ATOM | 2288 | OH2 | TIP | S | 106 | −18.087 | 70.972 | −46.270 | 1.00 | 66.12 | S |
| ATOM | 2289 | OH2 | TIP | S | 107 | −43.366 | 115.216 | −64.598 | 1.00 | 88.46 | S |
| ATOM | 2290 | OH2 | TIP | S | 108 | −49.436 | 89.111 | −72.407 | 1.00 | 59.04 | S |
| ATOM | 2291 | OH2 | TIP | S | 109 | −24.747 | 104.973 | −79.131 | 1.00 | 94.41 | S |
| ATOM | 2292 | OH2 | TIP | S | 110 | −14.831 | 88.579 | −50.161 | 1.00 | 51.01 | S |
| ATOM | 2293 | OH2 | TIP | S | 111 | −20.775 | 93.910 | −63.673 | 1.00 | 67.89 | S |
| ATOM | 2294 | OH2 | TIP | S | 112 | −27.551 | 89.850 | −85.485 | 1.00 | 62.96 | S |
| ATOM | 2295 | OH2 | TIP | S | 113 | −9.610 | 113.671 | −56.246 | 1.00 | 64.18 | S |
| ATOM | 2296 | OH2 | TIP | S | 114 | −13.054 | 116.811 | −83.710 | 1.00 | 88.12 | S |
| ATOM | 2297 | OH2 | TIP | S | 115 | −36.908 | 72.261 | −51.629 | 1.00 | 52.10 | S |
| ATOM | 2298 | OH2 | TIP | S | 117 | −6.237 | 137.377 | −67.390 | 1.00 | 52.76 | 5 |
| ATOM | 2299 | OH2 | TIP | S | 118 | −41.941 | 80.782 | −57.338 | 1.00 | 73.11 | S |
| ATOM | 2300 | OH2 | TIP | S | 119 | −10.771 | 115.633 | −81.759 | 1.00 | 85.64 | S |
| ATOM | 2301 | OH2 | TIP | S | 120 | −8.339 | 89.553 | −70.396 | 1.00 | 53.46 | S |
| ATOM | 2302 | OH2 | TIP | S | 122 | −48.014 | 76.217 | −64.376 | 1.00 | 45.72 | S |
| ATOM | 2303 | OH2 | TIP | S | 123 | −13.904 | 127.700 | −55.955 | 1.00 | 55.62 | S |
| ATOM | 2304 | OH2 | TIP | S | 124 | −17.611 | 112.118 | −90.078 | 1.00 | 65.69 | S |
| ATOM | 2305 | OH2 | TIP | S | 125 | −50.470 | 95.953 | −55.357 | 1.00 | 54.87 | S |
| END | | | | | | | | | | | |

TABLE 5
Coordinate data for ΔPH-PKBβ

CRYST1 149.523 149.523 39.055 90.00 90.00 90.00 P 41 21 2
REMARK FILENAME = “dph-pkb.pdb”
REMARK DATE: 23-Nov-01 19:09:37
REMARK VERSION: 1.1

| | | | | | | | | | | | |
|---|---|---|---|---|---|---|---|---|---|---|---|
| ATOM | 1 | CB | ALA | | 146 | −38.038 | 81.194 | −46.909 | 1.00 | 73.30 | |
| ATOM | 2 | C | ALA | | 146 | −38.368 | 81.486 | −49.380 | 1.00 | 72.38 | |
| ATOM | 3 | O | ALA | | 146 | −38.974 | 80.443 | −49.651 | 1.00 | 72.14 | |
| ATOM | 4 | N | ALA | | 146 | −39.973 | 82.494 | −47.814 | 1.00 | 72.57 | |
| ATOM | 5 | CA | ALA | | 146 | −38.532 | 82.146 | −48.018 | 1.00 | 73.03 | |
| ATOM | 6 | N | ALA | | 147 | −37.552 | 82.101 | −50.235 | 1.00 | 71.08 | |
| ATOM | 7 | CA | ALA | | 147 | −37.311 | 81.581 | −51.575 | 1.00 | 68.29 | |
| ATOM | 8 | CB | ALA | | 147 | −36.701 | 82.650 | −52.443 | 1.00 | 68.54 | |
| ATOM | 9 | C | ALA | | 147 | −36.379 | 80.400 | −51.494 | 1.00 | 67.18 | |
| ATOM | 10 | O | ALA | | 147 | −35.737 | 80.164 | −50.473 | 1.00 | 66.81 | |
| ATOM | 11 | N | THR | | 148 | −36.294 | 79.656 | −52.581 | 1.00 | 66.55 | |
| ATOM | 12 | CA | THR | | 148 | −35.418 | 78.506 | −52.610 | 1.00 | 66.31 | |
| ATOM | 13 | CB | THR | | 148 | −36.000 | 77.352 | −51.749 | 1.00 | 67.26 | |
| ATOM | 14 | OG1 | THR | | 148 | −35.147 | 77.143 | −50.621 | 1.00 | 67.64 | |
| ATOM | 15 | CG2 | THR | | 148 | −36.119 | 76.049 | −52.542 | 1.00 | 66.47 | |
| ATOM | 16 | C | THR | | 148 | −35.204 | 78.058 | −54.039 | 1.00 | 66.45 | |
| ATOM | 17 | O | THR | | 148 | −35.996 | 78.380 | −54.926 | 1.00 | 66.16 | |
| ATOM | 18 | N | MET | | 149 | −34.124 | 77.317 | −54.251 | 1.00 | 65.81 | |
| ATOM | 19 | CA | MET | | 149 | −33.780 | 76.809 | −55.564 | 1.00 | 66.24 | |
| ATOM | 20 | CB | MET | | 149 | −32.709 | 75.732 | −55.436 | 1.00 | 65.07 | |
| ATOM | 21 | CG | MET | | 149 | −31.299 | 76.285 | −55.380 | 1.00 | 63.77 | |
| ATOM | 22 | SD | MET | | 149 | −30.919 | 77.222 | −56.876 | 1.00 | 63.06 | |
| ATOM | 23 | CE | MET | | 149 | −29.920 | 78.511 | −56.204 | 1.00 | 63.24 | |
| ATOM | 24 | C | MET | | 149 | −34.965 | 76.251 | −56.326 | 1.00 | 67.08 | |
| ATOM | 25 | O | MET | | 149 | −35.168 | 76.573 | −57.494 | 1.00 | 67.93 | |
| ATOM | 26 | N | ASN | | 150 | −35.759 | 75.429 | −55.651 | 1.00 | 68.71 | |
| ATOM | 27 | CA | ASN | | 150 | −36.910 | 74.785 | −56.265 | 1.00 | 68.51 | |
| ATOM | 28 | CB | ASN | | 150 | −37.387 | 73.659 | −55.361 | 1.00 | 70.67 | |
| ATOM | 29 | CG | ASN | | 150 | −38.132 | 72.592 | −56.118 | 1.00 | 74.10 | |
| ATOM | 30 | OD1 | ASN | | 150 | −38.586 | 71.614 | −55.533 | 1.00 | 77.27 | |
| ATOM | 31 | ND2 | ASN | | 150 | −38.260 | 72.767 | −57.433 | 1.00 | 76.43 | |
| ATOM | 32 | C | ASN | | 150 | −38.085 | 75.701 | −56.616 | 1.00 | 67.77 | |
| ATOM | 33 | O | ASN | | 150 | −39.083 | 75.248 | −57.183 | 1.00 | 67.35 | |
| ATOM | 34 | N | ASP | | 151 | −37.982 | 76.981 | −56.276 | 1.00 | 65.96 | |
| ATOM | 35 | CA | ASP | | 151 | −39.049 | 77.919 | −56.595 | 1.00 | 65.30 | |
| ATOM | 36 | CB | ASP | | 151 | −39.020 | 79.128 | −55.652 | 1.00 | 66.76 | |
| ATOM | 37 | CG | ASP | | 151 | −39.490 | 78.802 | −54.238 | 1.00 | 68.61 | |
| ATOM | 38 | OD1 | ASP | | 151 | −40.554 | 78.164 | −54.090 | 1.00 | 69.24 | |
| ATOM | 39 | OD2 | ASP | | 151 | −38.807 | 79.206 | −53.267 | 1.00 | 70.04 | |
| ATOM | 40 | C | ASP | | 151 | −38.909 | 78.439 | −58.023 | 1.00 | 64.65 | |
| ATOM | 41 | O | ASP | | 151 | −39.786 | 79.147 | −58.506 | 1.00 | 65.43 | |
| ATOM | 42 | N | PHE | | 152 | −37.822 | 78.086 | −58.706 | 1.00 | 63.65 | |
| ATOM | 43 | CA | PHE | | 152 | −37.579 | 78.603 | −60.052 | 1.00 | 62.25 | |
| ATOM | 44 | CB | PHE | | 152 | −36.379 | 79.565 | −60.044 | 1.00 | 60.50 | |
| ATOM | 45 | CG | PHE | | 152 | −36.425 | 80.620 | −58.974 | 1.00 | 57.88 | |
| ATOM | 46 | CD1 | PHE | | 152 | −37.115 | 81.801 | −59.173 | 1.00 | 56.75 | |
| ATOM | 47 | CD2 | PHE | | 152 | −35.750 | 80.432 | −57.768 | 1.00 | 57.33 | |
| ATOM | 48 | CE1 | PHE | | 152 | −37.136 | 82.786 | −58.179 | 1.00 | 56.94 | |
| ATOM | 49 | CE2 | PHE | | 152 | −35.760 | 81.400 | −56.774 | 1.00 | 56.46 | |
| ATOM | 50 | CZ | PHE | | 152 | −36.452 | 82.579 | −56.978 | 1.00 | 57.58 | |
| ATOM | 51 | C | PHE | | 152 | −37.284 | 77.556 | −61.118 | 1.00 | 63.50 | |
| ATOM | 52 | O | PHE | | 152 | −36.883 | 76.426 | −60.816 | 1.00 | 64.19 | |
| ATOM | 53 | N | ASP | | 153 | −37.458 | 77.973 | −62.375 | 1.00 | 63.33 | |
| ATOM | 54 | CA | ASP | | 153 | −37.178 | 77.150 | −63.545 | 1.00 | 62.04 | |
| ATOM | 55 | CB | ASP | | 153 | −38.379 | 77.111 | −64.493 | 1.00 | 64.71 | |
| ATOM | 56 | CG | ASP | | 153 | −39.517 | 76.236 | −63.973 | 1.00 | 67.74 | |
| ATOM | 57 | OD1 | ASP | | 153 | −39.297 | 75.021 | −63.764 | 1.00 | 68.84 | |
| ATOM | 58 | OD2 | ASP | | 153 | −40.636 | 76.763 | −63.779 | 1.00 | 68.99 | |
| ATOM | 59 | C | ASP | | 153 | −35.992 | 77.774 | −64.272 | 1.00 | 60.87 | |
| ATOM | 60 | O | ASP | | 153 | −35.891 | 78.998 | −64.387 | 1.00 | 60.45 | |
| ATOM | 61 | N | TYR | | 154 | −35.099 | 76.925 | −64.760 | 1.00 | 58.73 | |
| ATOM | 62 | CA | TYR | | 154 | −33.918 | 77.366 | −65.475 | 1.00 | 57.74 | |
| ATOM | 63 | CB | TYR | | 154 | −32.856 | 76.282 | −65.405 | 1.00 | 57.78 | |
| ATOM | 64 | CG | TYR | | 154 | −31.632 | 76.579 | −66.248 | 1.00 | 58.77 | |
| ATOM | 65 | CD1 | TYR | | 154 | −30.660 | 77.485 | −65.813 | 1.00 | 59.49 | |
| ATOM | 66 | CE1 | TYR | | 154 | −29.515 | 77.711 | −66.553 | 1.00 | 60.96 | |
| ATOM | 67 | CD2 | TYR | | 154 | −31.423 | 75.918 | −67.451 | 1.00 | 57.76 | |
| ATOM | 68 | CE2 | TYR | | 154 | −30.278 | 76.133 | −68.193 | 1.00 | 59.31 | |
| ATOM | 69 | CZ | TYR | | 154 | −29.327 | 77.024 | −67.739 | 1.00 | 61.22 | |
| ATOM | 70 | OH | TYR | | 154 | −28.165 | 77.185 | −68.452 | 1.00 | 62.54 | |
| ATOM | 71 | C | TYR | | 154 | −34.146 | 77.694 | −66.949 | 1.00 | 57.85 | |
| ATOM | 72 | O | TYR | | 154 | −34.715 | 76.898 | −67.682 | 1.00 | 59.77 | |
| ATOM | 73 | N | LEU | | 155 | −33.678 | 78.849 | −67.405 | 1.00 | 57.45 | |
| ATOM | 74 | CA | LEU | | 155 | −33.836 | 79.179 | −68.816 | 1.00 | 55.82 | |
| ATOM | 75 | CB | LEU | | 155 | −34.554 | 80.519 | −68.981 | 1.00 | 53.85 | |
| ATOM | 76 | CG | LEU | | 155 | −35.982 | 80.397 | −68.459 | 1.00 | 54.19 | |
| ATOM | 77 | CD1 | LEU | | 155 | −36.671 | 81.722 | −68.473 | 1.00 | 53.47 | |
| ATOM | 78 | CD2 | LEU | | 155 | −36.738 | 79.377 | −69.306 | 1.00 | 54.40 | |
| ATOM | 79 | C | LEU | | 155 | −32.498 | 79.199 | −69.527 | 1.00 | 55.26 | |
| ATOM | 80 | O | LEU | | 155 | −32.247 | 78.384 | −70.412 | 1.00 | 53.81 | |
| ATOM | 81 | N | LYS | | 156 | −31.622 | 80.111 | −69.125 | 1.00 | 56.86 | |
| ATOM | 82 | CA | LYS | | 156 | −30.319 | 80.210 | −69.774 | 1.00 | 57.99 | |
| ATOM | 83 | CB | LYS | | 156 | −30.438 | 81.052 | −71.056 | 1.00 | 58.45 | |
| ATOM | 84 | CG | LYS | | 156 | −31.162 | 82.394 | −70.854 | 1.00 | 59.97 | |
| ATOM | 85 | CD | LYS | | 156 | −31.156 | 83.250 | −72.125 | 1.00 | 60.20 | |
| ATOM | 86 | CE | LYS | | 156 | −29.778 | 83.830 | −72.408 | 1.00 | 59.49 | |
| ATOM | 87 | NZ | LYS | | 156 | −29.771 | 84.739 | −73.587 | 1.00 | 60.88 | |
| ATOM | 88 | C | LYS | | 156 | −29.213 | 80.796 | −68.914 | 1.00 | 57.53 | |
| ATOM | 89 | O | LYS | | 156 | −29.447 | 81.368 | −67.843 | 1.00 | 56.57 | |
| ATOM | 90 | N | LEU | | 157 | −28.000 | 80.644 | −69.418 | 1.00 | 57.95 | |
| ATOM | 91 | CA | LEU | | 157 | −26.819 | 81.162 | −68.764 | 1.00 | 60.12 | |
| ATOM | 92 | CB | LEU | | 157 | −25.604 | 80.336 | −69.161 | 1.00 | 59.27 | |
| ATOM | 93 | CG | LEU | | 157 | −24.256 | 80.791 | −68.607 | 1.00 | 58.35 | |
| ATOM | 94 | CD1 | LEU | | 157 | −24.330 | 80.986 | −67.101 | 1.00 | 57.36 | |
| ATOM | 95 | CD2 | LEU | | 157 | −23.211 | 79.742 | −68.966 | 1.00 | 58.09 | |
| ATOM | 96 | C | LEU | | 157 | −26.644 | 82.592 | −69.244 | 1.00 | 61.72 | |
| ATOM | 97 | O | LEU | | 157 | −26.534 | 82.826 | −70.441 | 1.00 | 62.13 | |
| ATOM | 98 | N | LEU | | 158 | −26.632 | 83.541 | −68.311 | 1.00 | 63.84 | |
| ATOM | 99 | CA | LEU | | 158 | −26.480 | 84.954 | −68.647 | 1.00 | 65.91 | |
| ATOM | 100 | CB | LEU | | 158 | −27.291 | 85.832 | −67.689 | 1.00 | 64.45 | |
| ATOM | 101 | CG | LEU | | 158 | −28.814 | 85.750 | −67.821 | 1.00 | 64.01 | |
| ATOM | 102 | CD1 | LEU | | 158 | −29.457 | 86.404 | −66.607 | 1.00 | 64.00 | |
| ATOM | 103 | CD2 | LEU | | 158 | −29.270 | 86.421 | −69.109 | 1.00 | 61.97 | |
| ATOM | 104 | C | LEU | | 158 | −25.024 | 85.359 | −68.582 | 1.00 | 67.50 | |
| ATOM | 105 | O | LEU | | 158 | −24.566 | 86.154 | −69.385 | 1.00 | 68.41 | |
| ATOM | 106 | N | GLY | | 159 | −24.298 | 84.804 | −67.625 | 1.00 | 70.15 | |
| ATOM | 107 | CA | GLY | | 159 | −22.895 | 85.139 | −67.494 | 1.00 | 74.03 | |
| ATOM | 108 | C | GLY | | 159 | −22.215 | 84.276 | −66.456 | 1.00 | 76.35 | |
| ATOM | 109 | O | GLY | | 159 | −22.840 | 83.849 | −65.490 | 1.00 | 76.38 | |
| ATOM | 110 | N | LYS | | 160 | −20.934 | 84.006 | −66.657 | 1.00 | 79.22 | |
| ATOM | 111 | CA | LYS | | 160 | −20.186 | 83.188 | −65.710 | 1.00 | 82.44 | |
| ATOM | 112 | CB | LYS | | 160 | −19.600 | 81.968 | −66.428 | 1.00 | 83.26 | |
| ATOM | 113 | CG | LYS | | 160 | −19.286 | 80.795 | −65.512 | 1.00 | 85.18 | |
| ATOM | 114 | CD | LYS | | 160 | −18.705 | 79.613 | −66.283 | 1.00 | 86.35 | |
| ATOM | 115 | CE | LYS | | 160 | −18.593 | 78.367 | −65.396 | 1.00 | 87.69 | |
| ATOM | 116 | NZ | LYS | | 160 | −17.868 | 77.240 | −66.058 | 1.00 | 87.72 | |
| ATOM | 117 | C | LYS | | 160 | −19.072 | 84.052 | −65.100 | 1.00 | 84.24 | |
| ATOM | 118 | O | LYS | | 160 | −19.025 | 85.263 | −65.340 | 1.00 | 84.74 | |
| ATOM | 119 | N | GLY | | 161 | −18.189 | 83.452 | −64.304 | 1.00 | 85.47 | |
| ATOM | 120 | CA | GLY | | 161 | −17.116 | 84.235 | −63.715 | 1.00 | 86.79 | |
| ATOM | 121 | C | GLY | | 161 | −16.253 | 83.558 | −62.666 | 1.00 | 87.65 | |
| ATOM | 122 | O | GLY | | 161 | −16.388 | 82.366 | −62.394 | 1.00 | 87.66 | |
| ATOM | 123 | N | THR | | 162 | −15.352 | 84.341 | −62.079 | 1.00 | 88.86 | |
| ATOM | 124 | CA | THR | | 162 | −14.442 | 83.859 | −61.042 | 1.00 | 89.23 | |
| ATOM | 125 | CB | THR | | 162 | −13.109 | 84.639 | −61.078 | 1.00 | 90.45 | |
| ATOM | 126 | OG1 | THR | | 162 | −12.755 | 84.919 | −62.442 | 1.00 | 91.39 | |
| ATOM | 127 | CG2 | THR | | 162 | −12.000 | 83.821 | −60.438 | 1.00 | 90.70 | |
| ATOM | 128 | C | THR | | 162 | −15.119 | 84.083 | −59.688 | 1.00 | 88.56 | |
| ATOM | 129 | O | THR | | 162 | −14.593 | 83.718 | −58.635 | 1.00 | 89.01 | |
| ATOM | 130 | N | PHE | | 163 | −16.298 | 84.694 | −59.743 | 1.00 | 87.18 | |
| ATOM | 131 | CA | PHE | | 163 | −17.098 | 84.983 | −58.562 | 1.00 | 85.60 | |
| ATOM | 132 | CB | PHE | | 163 | −17.620 | 86.409 | −58.663 | 1.00 | 86.54 | |
| ATOM | 133 | CG | PHE | | 163 | −18.367 | 86.686 | −59.943 | 1.00 | 88.12 | |
| ATOM | 134 | CD1 | PHE | | 163 | −19.706 | 86.313 | −60.083 | 1.00 | 88.11 | |
| ATOM | 135 | CD2 | PHE | | 163 | −17.729 | 87.308 | −61.013 | 1.00 | 88.99 | |
| ATOM | 136 | CE1 | PHE | | 163 | −20.403 | 86.560 | −61.262 | 1.00 | 88.63 | |
| ATOM | 137 | CE2 | PHE | | 163 | −18.416 | 87.561 | −62.201 | 1.00 | 89.91 | |
| ATOM | 138 | CZ | PHE | | 163 | −19.761 | 87.183 | −62.325 | 1.00 | 89.59 | |
| ATOM | 139 | C | PHE | | 163 | −18.269 | 83.990 | −58.512 | 1.00 | 83.93 | |
| ATOM | 140 | O | PHE | | 163 | −18.797 | 83.678 | −57.441 | 1.00 | 82.94 | |
| ATOM | 141 | N | GLY | | 164 | −18.654 | 83.499 | −59.691 | 1.00 | 81.62 | |
| ATOM | 142 | CA | GLY | | 164 | −19.751 | 82.552 | −59.807 | 1.00 | 78.46 | |
| ATOM | 143 | C | GLY | | 164 | −20.414 | 82.557 | −61.181 | 1.00 | 75.60 | |
| ATOM | 144 | O | GLY | | 164 | −19.757 | 82.319 | −62.203 | 1.00 | 75.33 | |
| ATOM | 145 | N | LYS | | 165 | −21.721 | 82.808 | −61.212 | 1.00 | 72.45 | |
| ATOM | 46 | CA | LYS | | 165 | −22.457 | 82.844 | −62.471 | 1.00 | 69.28 | |
| ATOM | 147 | CB | LYS | | 165 | −22.642 | 81.420 | −63.023 | 1.00 | 69.65 | |
| ATOM | 148 | CG | LYS | | 165 | −23.791 | 80.627 | −62.431 | 1.00 | 70.80 | |
| ATOM | 149 | CD | LYS | | 165 | −23.723 | 79.153 | −62.824 | 1.00 | 73.65 | |
| ATOM | 150 | CE | LYS | | 165 | −22.523 | 78.462 | −62.159 | 1.00 | 75.99 | |
| ATOM | 151 | NZ | LYS | | 165 | −22.550 | 76.965 | −62.206 | 1.00 | 75.49 | |
| ATOM | 152 | C | LYS | | 165 | −23.818 | 83.521 | −62.316 | 1.00 | 67.03 | |
| ATOM | 153 | O | LYS | | 165 | −24.328 | 83.676 | −61.205 | 1.00 | 65.82 | |
| ATOM | 154 | N | VAL | | 166 | −24.386 | 83.940 | −63.442 | 1.00 | 64.70 | |
| ATOM | 155 | CA | VAL | | 166 | −25.697 | 84.579 | −63.460 | 1.00 | 62.03 | |
| ATOM | 156 | CB | VAL | | 166 | −25.633 | 86.040 | −63.936 | 1.00 | 60.81 | |
| ATOM | 157 | CG1 | VAL | | 166 | −26.987 | 86.689 | −63.762 | 1.00 | 60.12 | |
| ATOM | 158 | CG2 | VAL | | 166 | −24.573 | 86.800 | −63.163 | 1.00 | 59.49 | |
| ATOM | 159 | C | VAL | | 166 | −26.553 | 83.780 | −64.430 | 1.00 | 61.42 | |
| ATOM | 160 | O | VAL | | 166 | −26.161 | 83.530 | −65.570 | 1.00 | 59.35 | |
| ATOM | 161 | N | ILE | | 167 | −27.728 | 83.389 | −63.957 | 1.00 | 61.73 | |
| ATOM | 162 | CA | ILE | | 167 | −28.656 | 82.581 | −64.730 | 1.00 | 61.19 | |
| ATOM | 163 | CB | ILE | | 167 | −28.970 | 81.289 | −63.979 | 1.00 | 60.19 | |
| ATOM | 164 | CG2 | ILE | | 167 | −29.944 | 80.464 | −64.768 | 1.00 | 61.63 | |
| ATOM | 165 | CG1 | ILE | | 167 | −27.685 | 80.518 | −63.696 | 1.00 | 60.81 | |
| ATOM | 166 | CD1 | ILE | | 167 | −26.997 | 79.990 | −64.930 | 1.00 | 61.62 | |
| ATOM | 167 | C | ILE | | 167 | −29.975 | 83.297 | −64.945 | 1.00 | 61.83 | |
| ATOM | 168 | O | ILE | | 167 | −30.381 | 84.128 | −64.134 | 1.00 | 63.36 | |
| ATOM | 169 | N | LEU | | 168 | −30.651 | 82.969 | −66.036 | 1.00 | 61.16 | |
| ATOM | 170 | CA | LEU | | 168 | −31.957 | 83.553 | −66.313 | 1.00 | 59.71 | |
| ATOM | 171 | CB | LEU | | 168 | −32.152 | 83.764 | −67.820 | 1.00 | 59.59 | |
| ATOM | 172 | CG | LEU | | 168 | −33.463 | 84.396 | −68.300 | 1.00 | 59.38 | |
| ATOM | 173 | CD1 | LEU | | 168 | −33.747 | 85.672 | −67.521 | 1.00 | 58.51 | |
| ATOM | 174 | CD2 | LEU | | 168 | −33.366 | 84.690 | −69.790 | 1.00 | 60.52 | |
| ATOM | 175 | C | LEU | | 168 | −32.934 | 82.506 | −65.807 | 1.00 | 59.39 | |
| ATOM | 176 | O | LEU | | 168 | −32.858 | 81.344 | −66.210 | 1.00 | 60.09 | |
| ATOM | 177 | N | VAL | | 169 | −33.830 | 82.900 | −64.908 | 1.00 | 58.74 | |
| ATOM | 178 | CA | VAL | | 169 | −34.819 | 81.967 | −64.367 | 1.00 | 57.54 | |
| ATOM | 179 | CB | VAL | | 169 | −34.457 | 81.502 | −62.931 | 1.00 | 56.13 | |
| ATOM | 180 | CG1 | VAL | | 169 | −33.076 | 80.892 | −62.916 | 1.00 | 53.50 | |
| ATOM | 181 | CG2 | VAL | | 169 | −34.544 | 82.668 | −61.965 | 1.00 | 52.21 | |
| ATOM | 182 | C | VAL | | 169 | −36.215 | 82.566 | −64.319 | 1.00 | 58.69 | |
| ATOM | 183 | O | VAL | | 169 | −36.400 | 83.778 | −64.458 | 1.00 | 56.03 | |
| ATOM | 184 | N | ARG | | 170 | −37.194 | 81.691 | −64.120 | 1.00 | 60.60 | |
| ATOM | 185 | CA | ARG | | 170 | −38.582 | 82.090 | −64.026 | 1.00 | 63.17 | |
| ATOM | 186 | CB | ARG | | 170 | −39.410 | 81.449 | −65.148 | 1.00 | 64.23 | |
| ATOM | 187 | CG | ARG | | 170 | −40.825 | 82.053 | −65.318 | 1.00 | 65.59 | |
| ATOM | 188 | CD | ARG | | 170 | −41.746 | 81.157 | −66.154 | 1.00 | 66.11 | |
| ATOM | 189 | NE | ARG | | 170 | −41.068 | 80.639 | −67.342 | 1.00 | 67.53 | |
| ATOM | 190 | CZ | ARG | | 170 | −40.827 | 79.347 | −67.572 | 1.00 | 67.14 | |
| ATOM | 191 | NH1 | ARG | | 170 | −41.213 | 78.427 | −66.693 | 1.00 | 66.28 | |
| ATOM | 192 | NH2 | ARG | | 170 | −40.194 | 78.976 | −68.682 | 1.00 | 66.92 | |
| ATOM | 193 | C | ARG | | 170 | −39.131 | 81.629 | −62.686 | 1.00 | 64.75 | |
| ATOM | 194 | O | ARG | | 170 | −39.107 | 80.439 | −62.385 | 1.00 | 65.77 | |
| ATOM | 195 | N | GLU | | 171 | −39.609 | 82.563 | −61.875 | 1.00 | 66.65 | |
| ATOM | 196 | CA | GLU | | 171 | −40.200 | 82.198 | −60.599 | 1.00 | 68.87 | |
| ATOM | 197 | CB | GLU | | 171 | −40.559 | 83.454 | −59.808 | 1.00 | 68.24 | |
| ATOM | 198 | CG | CLU | | 171 | −41.489 | 83.212 | −58.645 | 1.00 | 69.67 | |
| ATOM | 199 | CD | GLU | | 171 | −41.577 | 84.399 | −57.692 | 1.00 | 71.08 | |
| ATOM | 200 | OE1 | GLU | | 171 | −41.646 | 85.567 | −58.160 | 1.00 | 70.33 | |
| ATOM | 201 | OE2 | GLU | | 171 | −41.586 | 84.151 | −56.466 | 1.00 | 70.60 | |
| ATOM | 202 | C | GLU | | 171 | −41.451 | 81.415 | −60.981 | 1.00 | 71.35 | |
| ATOM | 203 | O | GLU | | 171 | −42.307 | 81.922 | −61.701 | 1.00 | 72.05 | |
| ATOM | 204 | N | LYS | | 172 | −41.552 | 80.171 | −60.529 | 1.00 | 73.13 | |
| ATOM | 205 | CA | LYS | | 172 | −42.703 | 79.355 | −60.875 | 1.00 | 74.23 | |
| ATOM | 206 | CB | LYS | | 172 | −42.483 | 77.922 | −60.399 | 1.00 | 74.29 | |
| ATOM | 207 | CG | LYS | | 172 | −41.471 | 77.214 | −61.274 | 1.00 | 75.99 | |
| ATOM | 208 | CD | LYS | | 172 | −41.241 | 75.757 | −60.912 | 1.00 | 76.76 | |
| ATOM | 209 | CE | LYS | | 172 | −40.397 | 75.598 | −59.660 | 1.00 | 76.33 | |
| ATOM | 210 | NZ | LYS | | 172 | −39.910 | 74.195 | −59.515 | 1.00 | 75.41 | |
| ATOM | 211 | C | LYS | | 172 | −44.037 | 79.891 | −60.379 | 1.00 | 75.60 | |
| ATOM | 212 | O | LYS | | 172 | −45.072 | 79.663 | −61.004 | 1.00 | 76.21 | |
| ATOM | 213 | N | ALA | | 173 | −44.023 | 80.612 | −59.265 | 1.00 | 77.07 | |
| ATOM | 214 | CA | ALA | | 173 | −45.258 | 81.174 | −58.729 | 1.00 | 78.78 | |
| ATOM | 215 | CB | ALA | | 173 | −45.016 | 81.745 | −57.330 | 1.00 | 78.63 | |
| ATOM | 216 | C | ALA | | 173 | −45.770 | 82.272 | −59.661 | 1.00 | 79.50 | |
| ATOM | 217 | O | ALA | | 173 | −46.722 | 82.068 | −60.419 | 1.00 | 79.63 | |
| ATOM | 218 | N | THR | | 174 | −45.106 | 83.427 | −59.610 | 1.00 | 79.51 | |
| ATOM | 219 | CA | THR | | 174 | −45.474 | 84.588 | −60.414 | 1.00 | 78.86 | |
| ATOM | 220 | CB | THR | | 174 | −44.739 | 85.874 | −59.929 | 1.00 | 79.44 | |
| ATOM | 221 | OG1 | THR | | 174 | −43.332 | 85.759 | −60.187 | 1.00 | 79.70 | |
| ATOM | 222 | CG2 | THR | | 174 | −44.965 | 86.097 | −58.437 | 1.00 | 79.83 | |
| ATOM | 223 | C | THR | | 174 | −45.237 | 84.476 | −61.918 | 1.00 | 78.33 | |
| ATOM | 224 | O | THR | | 174 | −45.724 | 85.306 | −62.675 | 1.00 | 79.01 | |
| ATOM | 225 | N | GLY | | 175 | −44.496 | 83.470 | −62.361 | 1.00 | 77.74 | |
| ATOM | 226 | CA | GLY | | 175 | −44.233 | 83.350 | −63.784 | 1.00 | 76.56 | |
| ATOM | 227 | C | GLY | | 175 | −43.262 | 84.417 | −64.268 | 1.00 | 76.35 | |
| ATOM | 228 | O | GLY | | 175 | −42.861 | 84.413 | −65.431 | 1.00 | 76.58 | |
| ATOM | 229 | N | ARG | | 176 | −42.881 | 85.323 | −63.370 | 1.00 | 75.54 | |
| ATOM | 230 | CA | ARG | | 176 | −41.957 | 86.411 | −63.680 | 1.00 | 75.88 | |
| ATOM | 231 | CB | ARG | | 176 | −41.906 | 87.392 | −62.505 | 1.00 | 79.56 | |
| ATOM | 232 | CG | ARG | | 176 | −43.242 | 88.006 | −62.109 | 1.00 | 84.08 | |
| ATOM | 233 | CD | ARG | | 176 | −43.796 | 88.927 | −63.190 | 1.00 | 88.26 | |
| ATOM | 234 | NE | ARG | | 176 | −45.072 | 89.523 | −62.788 | 1.00 | 92.52 | |
| ATOM | 235 | CZ | ARG | | 176 | −45.910 | 90.140 | −63.621 | 1.00 | 94.75 | |
| ATOM | 236 | NH1 | ARG | | 176 | −45.610 | 90.243 | −64.912 | 1.00 | 95.39 | |
| ATOM | 237 | NH2 | ARG | | 176 | −47.048 | 90.659 | −63.165 | 1.00 | 95.28 | |
| ATOM | 238 | C | ARG | | 176 | −40.524 | 85.936 | −63.997 | 1.00 | 74.64 | |
| ATOM | 239 | O | ARG | | 176 | −40.163 | 84.791 | −63.725 | 1.00 | 74.48 | |
| ATOM | 240 | N | TYR | | 177 | −39.712 | 86.836 | −64.556 | 1.00 | 71.66 | |
| ATOM | 241 | CA | TYR | | 177 | −38.330 | 86.529 | −64.915 | 1.00 | 68.53 | |
| ATOM | 242 | CB | TYR | | 177 | −38.078 | 86.842 | −66.390 | 1.00 | 69.30 | |
| ATOM | 243 | CG | TYR | | 177 | −38.757 | 85.900 | −67.346 | 1.00 | 71.43 | |
| ATOM | 244 | CD1 | TYR | | 177 | −40.141 | 85.889 | −67.483 | 1.00 | 73.14 | |
| ATOM | 245 | CE1 | TYR | | 177 | −40.770 | 85.008 | −68.358 | 1.00 | 73.22 | |
| ATOM | 246 | CD2 | TYR | | 177 | −38.018 | 85.007 | −68.110 | 1.00 | 72.26 | |
| ATOM | 247 | CE2 | TYR | | 177 | −38.640 | 84.126 | −68.986 | 1.00 | 73.17 | |
| ATOM | 248 | CZ | TYR | | 177 | −40.012 | 84.131 | −69.102 | 1.00 | 73.66 | |
| ATOM | 249 | OH | TYR | | 177 | −40.625 | 83.249 | −69.958 | 1.00 | 75.02 | |
| ATOM | 250 | C | TYR | | 177 | −37.304 | 87.288 | −64.082 | 1.00 | 65.96 | |
| ATOM | 251 | O | TYR | | 177 | −37.422 | 88.490 | −63.863 | 1.00 | 66.11 | |
| ATOM | 252 | N | TYR | | 178 | −36.284 | 86.575 | −63.626 | 1.00 | 62.93 | |
| ATOM | 253 | CA | TYR | | 178 | −35.226 | 87.176 | −62.833 | 1.00 | 60.28 | |
| ATOM | 254 | CB | TYR | | 178 | −35.401 | 86.848 | −61.346 | 1.00 | 60.14 | |
| ATOM | 255 | CG | TYR | | 178 | −36.695 | 87.370 | −60.759 | 1.00 | 60.36 | |
| ATOM | 256 | CD1 | TYR | | 178 | −37.903 | 86.683 | −60.948 | 1.00 | 59.37 | |
| ATOM | 257 | CE1 | TYR | | 178 | −39.101 | 87.203 | −60.482 | 1.00 | 59.43 | |
| ATOM | 258 | CD2 | TYR | | 178 | −36.726 | 88.591 | −60.078 | 1.00 | 59.40 | |
| ATOM | 259 | CE2 | TYR | | 178 | −37.917 | 89.119 | −59.609 | 1.00 | 60.14 | |
| ATOM | 260 | CZ | TYR | | 178 | −39.100 | 88.422 | −59.816 | 1.00 | 60.79 | |
| ATOM | 261 | OH | TYR | | 178 | −40.279 | 88.963 | −59.366 | 1.00 | 64.02 | |
| ATOM | 262 | C | TYR | | 178 | −33.900 | 86.642 | −63.311 | 1.00 | 58.68 | |
| ATOM | 263 | O | TYR | | 178 | −33.844 | 85.715 | −64.107 | 1.00 | 56.28 | |
| ATOM | 264 | N | ALA | | 179 | −32.835 | 87.261 | −62.834 | 1.00 | 59.19 | |
| ATOM | 265 | CA | ALA | | 179 | −31.483 | 86.849 | −63.167 | 1.00 | 61.40 | |
| ATOM | 266 | CB | ALA | | 179 | −30.692 | 88.012 | −63.758 | 1.00 | 60.64 | |
| ATOM | 267 | C | ALA | | 179 | −30.897 | 86.427 | −61.835 | 1.00 | 63.02 | |
| ATOM | 268 | O | ALA | | 179 | −30.706 | 87.252 | −60.938 | 1.00 | 65.07 | |
| ATOM | 269 | N | MET | | 180 | −30.632 | 85.137 | −61.695 | 1.00 | 63.09 | |
| ATOM | 270 | CA | MET | | 180 | −30.103 | 84.617 | −60.444 | 1.00 | 63.02 | |
| ATOM | 271 | CB | MET | | 180 | −30.634 | 83.202 | −60.223 | 1.00 | 61.42 | |
| ATOM | 272 | CG | MET | | 180 | −30.405 | 82.667 | −58.837 | 1.00 | 61.56 | |
| ATOM | 273 | SD | MET | | 180 | −30.840 | 80.947 | −58.736 | 1.00 | 60.07 | |
| ATOM | 274 | CE | MET | | 180 | −32.520 | 81.084 | −58.339 | 1.00 | 61.93 | |
| ATOM | 275 | C | MET | | 180 | −28.575 | 84.624 | −60.398 | 1.00 | 63.37 | |
| ATOM | 276 | O | MET | | 180 | −27.924 | 83.813 | −61.053 | 1.00 | 64.64 | |
| ATOM | 277 | N | LYS | | 181 | −28.011 | 85.554 | −59.634 | 1.00 | 63.37 | |
| ATOM | 278 | CA | LYS | | 181 | −26.565 | 85.655 | −59.485 | 1.00 | 63.94 | |
| ATOM | 279 | CB | LYS | | 181 | −26.171 | 87.067 | −59.034 | 1.00 | 64.76 | |
| ATOM | 280 | CG | LYS | | 181 | −24.668 | 87.288 | −58.867 | 1.00 | 67.65 | |
| ATOM | 281 | CD | LYS | | 181 | −24.298 | 88.765 | −58.618 | 1.00 | 68.44 | |
| ATOM | 282 | CE | LYS | | 181 | −22.782 | 88.973 | −58.688 | 1.00 | 69.54 | |
| ATOM | 283 | NZ | LYS | | 181 | −22.348 | 90.393 | −58.617 | 1.00 | 69.40 | |
| ATOM | 284 | C | LYS | | 181 | −26.174 | 84.633 | −58.420 | 1.00 | 64.61 | |
| ATOM | 285 | O | LYS | | 181 | −26.638 | 84.709 | −57.279 | 1.00 | 64.31 | |
| ATOM | 286 | N | ILE | | 182 | −25.334 | 83.672 | −58.794 | 1.00 | 64.41 | |
| ATOM | 287 | CA | ILE | | 182 | −24.908 | 82.627 | −57.866 | 1.00 | 65.06 | |
| ATOM | 288 | CB | ILE | | 182 | −25.222 | 81.227 | −58.447 | 1.00 | 63.16 | |
| ATOM | 289 | CG2 | ILE | | 182 | −25.096 | 80.166 | −57.359 | 1.00 | 63.49 | |
| ATOM | 290 | CG1 | ILE | | 182 | −26.653 | 81.202 | −58.994 | 1.00 | 61.58 | |
| ATOM | 291 | CD1 | ILE | | 182 | −27.053 | 79.869 | −59.563 | 1.00 | 58.80 | |
| ATOM | 292 | C | ILE | | 182 | −23.412 | 82.712 | −57.532 | 1.00 | 66.90 | |
| ATOM | 293 | O | ILE | | 182 | −22.560 | 82.616 | −58.413 | 1.00 | 66.62 | |
| ATOM | 294 | N | LEU | | 183 | −23.098 | 82.873 | −56.252 | 1.00 | 68.62 | |
| ATOM | 295 | CA | LEU | | 183 | −21.707 | 82.985 | −55.820 | 1.00 | 71.25 | |
| ATOM | 296 | CB | LEU | | 183 | −21.515 | 84.307 | −55.079 | 1.00 | 70.08 | |
| ATOM | 297 | CG | LEU | | 183 | −21.909 | 85.553 | −55.859 | 1.00 | 70.20 | |
| ATOM | 298 | CD1 | LEU | | 183 | −21.690 | 86.766 | −54.988 | 1.00 | 69.25 | |
| ATOM | 299 | CD2 | LEU | | 183 | −21.089 | 85.643 | −57.136 | 1.00 | 69.20 | |
| ATOM | 300 | C | LEU | | 183 | −21.210 | 81.848 | −54.920 | 1.00 | 73.26 | |
| ATOM | 301 | O | LEU | | 183 | −21.897 | 81.450 | −53.980 | 1.00 | 72.60 | |
| ATOM | 302 | N | ARG | | 184 | −20.011 | 81.341 | −55.206 | 1.00 | 76.22 | |
| ATOM | 303 | CA | ARG | | 184 | −19.419 | 80.281 | −54.388 | 1.00 | 80.60 | |
| ATOM | 304 | CB | ARG | | 184 | −18.155 | 79.713 | −55.050 | 1.00 | 80.79 | |
| ATOM | 305 | CG | ARG | | 184 | −18.268 | 79.293 | −56.510 | 1.00 | 83.12 | |
| ATOM | 306 | CD | ARG | | 184 | −18.797 | 77.878 | −56.657 | 1.00 | 84.80 | |
| ATOM | 307 | NE | ARG | | 184 | −18.527 | 77.272 | −57.971 | 1.00 | 87.21 | |
| ATOM | 308 | CZ | ARG | | 184 | −18.831 | 77.815 | −59.152 | 1.00 | 88.61 | |
| ATOM | 309 | NH1 | ARG | | 184 | −19.418 | 79.005 | −59.231 | 1.00 | 89.57 | |
| ATOM | 310 | NH2 | ARG | | 184 | −18.572 | 77.148 | −60.268 | 1.00 | 88.97 | |
| ATOM | 311 | C | ARG | | 184 | −19.011 | 80.912 | −53.044 | 1.00 | 82.32 | |
| ATOM | 312 | O | ARG | | 184 | −18.384 | 81.969 | −53.029 | 1.00 | 82.47 | |
| ATOM | 313 | N | LYS | | 185 | −19.375 | 80.285 | −51.925 | 1.00 | 84.93 | |
| ATOM | 314 | CA | LYS | | 185 | −18.993 | 80.798 | −50.606 | 1.00 | 87.47 | |
| ATOM | 315 | CB | LYS | | 185 | −19.742 | 80.056 | −49.497 | 1.00 | 86.74 | |
| ATOM | 316 | CG | LYS | | 185 | −21.219 | 80.385 | −49.403 | 1.00 | 88.14 | |
| ATOM | 317 | CD | LYS | | 185 | −21.881 | 79.652 | −48.230 | 1.00 | 88.29 | |
| ATOM | 318 | CE | LYS | | 185 | −23.367 | 79.996 | −48.113 | 1.00 | 88.05 | |
| ATOM | 319 | NZ | LYS | | 185 | −24.098 | 79.175 | −47.103 | 1.00 | 86.65 | |
| ATOM | 320 | C | LYS | | 185 | −17.489 | 80.550 | −50.457 | 1.00 | 89.82 | |
| ATOM | 321 | O | LYS | | 185 | −16.768 | 81.280 | −49.760 | 1.00 | 89.49 | |
| ATOM | 322 | N | GLU | | 186 | −17.050 | 79.498 | −51.144 | 1.00 | 91.98 | |
| ATOM | 323 | CA | GLU | | 186 | −15.667 | 79.043 | −51.184 | 1.00 | 93.99 | |
| ATOM | 324 | CB | GLU | | 186 | −15.618 | 77.738 | −51.989 | 1.00 | 95.74 | |
| ATOM | 325 | CG | GLU | | 186 | −14.247 | 77.095 | −52.150 | 1.00 | 97.28 | |
| ATOM | 326 | CD | GLU | | 186 | −14.316 | 75.782 | −52.925 | 1.00 | 97.52 | |
| ATOM | 327 | OE1 | GLU | | 186 | −14.841 | 75.784 | −54.064 | 1.00 | 97.04 | |
| ATOM | 328 | OE2 | GLU | | 186 | −13.845 | 74.752 | −52.391 | 1.00 | 97.90 | |
| ATOM | 329 | C | GLU | | 186 | −14.751 | 80.101 | −51.808 | 1.00 | 94.24 | |
| ATOM | 330 | O | GLU | | 186 | −13.632 | 80.311 | −51.346 | 1.00 | 94.97 | |
| ATOM | 331 | N | VAL | | 187 | −15.227 | 80.760 | −52.859 | 1.00 | 94.74 | |
| ATOM | 332 | CA | VAL | | 187 | −14.451 | 81.806 | −53.525 | 1.00 | 95.50 | |
| ATOM | 333 | CB | VAL | | 187 | −15.155 | 82.287 | −54.813 | 1.00 | 95.76 | |
| ATOM | 334 | CG1 | VAL | | 187 | −14.562 | 83.618 | −55.277 | 1.00 | 95.48 | |
| ATOM | 335 | CG2 | VAL | | 187 | −15.014 | 81.225 | −55.902 | 1.00 | 95.91 | |
| ATOM | 336 | C | VAL | | 187 | −14.249 | 82.999 | −52.592 | 1.00 | 95.59 | |
| ATOM | 337 | O | VAL | | 187 | −13.149 | 83.538 | −52.495 | 1.00 | 95.85 | |
| ATOM | 338 | N | ILE | | 188 | −15.317 | 83.405 | −51.912 | 1.00 | 95.86 | |
| ATOM | 339 | CA | ILE | | 188 | −15.251 | 84.520 | −50.974 | 1.00 | 96.27 | |
| ATOM | 340 | CB | ILE | | 188 | −16.533 | 85.390 | −51.038 | 1.00 | 96.69 | |
| ATOM | 341 | CG2 | ILE | | 188 | −17.765 | 84.521 | −50.815 | 1.00 | 96.39 | |
| ATOM | 342 | CG1 | ILE | | 188 | −16.456 | 86.511 | −49.991 | 1.00 | 97.39 | |
| ATOM | 343 | CD1 | ILE | | 188 | −17.633 | 87.478 | −49.997 | 1.00 | 97.60 | |
| ATOM | 344 | C | ILE | | 188 | −15.082 | 84.000 | −49.543 | 1.00 | 96.43 | |
| ATOM | 345 | O | ILE | | 188 | −14.092 | 83.338 | −49.216 | 1.00 | 96.41 | |
| ATOM | 346 | N | ALA | | 198 | −18.432 | 91.977 | −49.977 | 1.00 | 117.70 | |
| ATOM | 347 | CA | ALA | | 198 | −19.657 | 92.421 | −49.318 | 1.00 | 117.91 | |
| ATOM | 348 | CB | ALA | | 198 | −19.440 | 92.483 | −47.809 | 1.00 | 117.84 | |
| ATOM | 349 | C | ALA | | 198 | −20.108 | 93.785 | −49.838 | 1.00 | 117.87 | |
| ATOM | 350 | O | ALA | | 198 | −21.090 | 94.347 | −49.353 | 1.00 | 116.79 | |
| ATOM | 351 | N | ALA | | 199 | −19.383 | 94.304 | −50.830 | 1.00 | 118.49 | |
| ATOM | 352 | CA | ALA | | 199 | −19.679 | 95.608 | −51.432 | 1.00 | 118.40 | |
| ATOM | 353 | CB | ALA | | 199 | −18.422 | 96.179 | −52.095 | 1.00 | 118.07 | |
| ATOM | 354 | C | ALA | | 199 | −20.810 | 95.530 | −52.452 | 1.00 | 118.10 | |
| ATOM | 355 | O | ALA | | 199 | −21.239 | 96.552 | −52.996 | 1.00 | 118.77 | |
| ATOM | 356 | N | ALA | | 200 | −21.279 | 94.314 | −52.717 | 1.00 | 117.30 | |
| ATOM | 357 | CA | ALA | | 200 | −22.372 | 94.091 | −53.660 | 1.00 | 116.57 | |
| ATOM | 358 | CB | ALA | | 200 | −21.991 | 92.987 | −54.656 | 1.00 | 116.07 | |
| ATOM | 359 | C | ALA | | 200 | −23.613 | 93.684 | −52.862 | 1.00 | 116.02 | |
| ATOM | 360 | O | ALA | | 200 | −24.727 | 94.145 | −53.124 | 1.00 | 115.51 | |
| ATOM | 361 | N | ALA | | 201 | −23.391 | 92.819 | −51.877 | 1.00 | 115.78 | |
| ATOM | 362 | CA | ALA | | 201 | −24.449 | 92.316 | −51.007 | 1.00 | 115.19 | |
| ATOM | 363 | CB | ALA | | 201 | −23.954 | 91.075 | −50.261 | 1.00 | 115.46 | |
| ATOM | 364 | C | ALA | | 201 | −24.886 | 93.387 | −50.008 | 1.00 | 114.36 | |
| ATOM | 365 | O | ALA | | 201 | −24.098 | 94.260 | −49.644 | 1.00 | 114.73 | |
| ATOM | 366 | N | ALA | | 202 | −26.139 | 93.305 | −49.564 | 1.00 | 112.87 | |
| ATOM | 367 | CA | ALA | | 202 | −26.701 | 94.261 | −48.610 | 1.00 | 110.91 | |
| ATOM | 368 | CB | ALA | | 202 | −25.936 | 94.196 | −47.292 | 1.00 | 110.82 | |
| ATOM | 369 | C | ALA | | 202 | −26.695 | 95.691 | −49.159 | 1.00 | 109.43 | |
| ATOM | 370 | O | ALA | | 202 | −27.748 | 96.316 | −49.280 | 1.00 | 108.79 | |
| ATOM | 371 | N | ALA | | 203 | −25.509 | 96.200 | −49.487 | 1.00 | 107.83 | |
| ATOM | 372 | CA | ALA | | 203 | −25.353 | 97.551 | −50.026 | 1.00 | 105.87 | |
| ATOM | 373 | CB | ALA | | 203 | −23.883 | 97.817 | −50.363 | 1.00 | 105.05 | |
| ATOM | 374 | C | ALA | | 203 | −26.216 | 97.731 | −51.272 | 1.00 | 104.62 | |
| ATOM | 375 | O | ALA | | 203 | −27.076 | 98.615 | −51.315 | 1.00 | 104.45 | |
| ATOM | 376 | N | ALA | | 204 | −25.980 | 96.888 | −52.278 | 1.00 | 102.74 | |
| ATOM | 377 | CA | ALA | | 204 | −26.733 | 96.939 | −53.527 | 1.00 | 100.15 | |
| ATOM | 378 | CB | ALA | | 204 | −25.961 | 96.229 | −54.628 | 1.00 | 99.98 | |
| ATOM | 379 | C | ALA | | 204 | −28.120 | 96.312 | −53.379 | 1.00 | 98.72 | |
| ATOM | 380 | O | ALA | | 204 | −28.937 | 96.392 | −54.290 | 1.00 | 98.28 | |
| ATOM | 381 | N | ALA | | 205 | −28.381 | 95.690 | −52.232 | 1.00 | 97.13 | |
| ATOM | 382 | CA | ALA | | 205 | −29.671 | 95.049 | −51.975 | 1.00 | 94.82 | |
| ATOM | 383 | CB | ALA | | 205 | −29.505 | 93.932 | −50.955 | 1.00 | 95.03 | |
| ATOM | 384 | C | ALA | | 205 | −30.721 | 96.040 | −51.480 | 1.00 | 93.24 | |
| ATOM | 385 | O | ALA | | 205 | −31.908 | 95.894 | −51.775 | 1.00 | 93.26 | |
| ATOM | 386 | N | ALA | | 206 | −30.279 | 97.045 | −50.726 | 1.00 | 90.80 | |
| ATOM | 387 | CA | ALA | | 206 | −31.183 | 98.053 | −50.178 | 1.00 | 87.61 | |
| ATOM | 388 | CB | ALA | | 206 | −30.801 | 98.360 | −48.721 | 1.00 | 87.13 | |
| ATOM | 389 | C | ALA | | 206 | −31.217 | 99.346 | −51.006 | 1.00 | 85.44 | |
| ATOM | 390 | O | ALA | | 206 | −32.114 | 100.179 | −50.834 | 1.00 | 85.50 | |
| ATOM | 391 | N | THR | | 207 | −30.248 | 99.520 | −51.899 | 1.00 | 82.56 | |
| ATOM | 392 | CA | THR | | 207 | −30.231 | 100.712 | −52.735 | 1.00 | 79.10 | |
| ATOM | 393 | CB | THR | | 207 | −28.823 | 101.026 | −53.286 | 1.00 | 79.91 | |
| ATOM | 394 | OG1 | THR | | 207 | −28.258 | 99.834 | −53.848 | 1.00 | 81.31 | |
| ATOM | 395 | CG2 | THR | | 207 | −27.918 | 101.576 | −52.185 | 1.00 | 77.84 | |
| ATOM | 396 | C | THR | | 207 | −31.184 | 100.490 | −53.900 | 1.00 | 76.04 | |
| ATOM | 397 | O | THR | | 207 | −30.847 | 99.845 | −54.897 | 1.00 | 76.19 | |
| ATOM | 398 | N | ARG | | 208 | −32.392 | 101.015 | −53.733 | 1.00 | 72.56 | |
| ATOM | 399 | CA | ARG | | 208 | −33.450 | 100.934 | −54.725 | 1.00 | 68.12 | |
| ATOM | 400 | CB | ARG | | 208 | −34.805 | 101.004 | −54.014 | 1.00 | 70.61 | |
| ATOM | 401 | CG | ARG | | 208 | −36.005 | 100.761 | −54.912 | 1.00 | 76.14 | |
| ATOM | 402 | CD | ARG | | 208 | −36.284 | 99.268 | −55.079 | 1.00 | 79.38 | |
| ATOM | 403 | NE | ARG | | 208 | −37.338 | 99.008 | −56.060 | 1.00 | 82.09 | |
| ATOM | 404 | CZ | ARG | | 208 | −37.984 | 97.849 | −56.187 | 1.00 | 82.92 | |
| ATOM | 405 | NH1 | ARG | | 208 | −37.696 | 96.822 | −55.392 | 1.00 | 81.74 | |
| ATOM | 406 | NH2 | ARG | | 208 | −38.922 | 97.716 | −57.116 | 1.00 | 84.03 | |
| ATOM | 407 | C | ARG | | 208 | −33.257 | 102.162 | −55.614 | 1.00 | 62.90 | |
| ATOM | 408 | O | ARG | | 208 | −32.694 | 103.146 | −55.163 | 1.00 | 62.14 | |
| ATOM | 409 | N | HIS | | 209 | −33.693 | 102.091 | −56.871 | 1.00 | 58.01 | |
| ATOM | 410 | CA | HIS | | 209 | −33.596 | 103.216 | −57.808 | 1.00 | 51.56 | |
| ATOM | 411 | CB | HIS | | 209 | −32.180 | 103.743 | −57.869 | 1.00 | 48.75 | |
| ATOM | 412 | CG | HIS | | 209 | −32.029 | 104.974 | −58.700 | 1.00 | 45.36 | |
| ATOM | 413 | CD2 | HIS | | 209 | −31.525 | 106.195 | −58.398 | 1.00 | 46.04 | |
| ATOM | 414 | ND1 | HIS | | 209 | −32.323 | 105.004 | −60.047 | 1.00 | 44.41 | |
| ATOM | 415 | CE1 | HIS | | 209 | −31.996 | 106.186 | −60.540 | 1.00 | 44.49 | |
| ATOM | 416 | NE2 | HIS | | 209 | −31.507 | 106.927 | −59.561 | 1.00 | 44.97 | |
| ATOM | 417 | C | HIS | | 209 | −34.051 | 102.883 | −59.224 | 1.00 | 49.66 | |
| ATOM | 418 | O | HIS | | 209 | −33.667 | 101.874 | −59.792 | 1.00 | 49.96 | |
| ATOM | 419 | N | PRO | | 210 | −34.884 | 103.746 | −59.813 | 1.00 | 48.82 | |
| ATOM | 420 | CD | PRO | | 210 | −35.383 | 104.998 | −59.215 | 1.00 | 48.63 | |
| ATOM | 421 | CA | PRO | | 210 | −35.419 | 103.594 | −61.163 | 1.00 | 47.87 | |
| ATOM | 422 | CB | PRO | | 210 | −35.758 | 105.026 | −61.544 | 1.00 | 46.95 | |
| ATOM | 423 | CG | PRO | | 210 | −36.306 | 105.535 | −60.293 | 1.00 | 47.55 | |
| ATOM | 424 | C | PRO | | 210 | −34.502 | 102.943 | −62.188 | 1.00 | 46.22 | |
| ATOM | 425 | O | PRO | | 210 | −34.944 | 102.085 | −63.941 | 1.00 | 46.47 | |
| ATOM | 426 | N | PHE | | 211 | −33.236 | 103.338 | −62.239 | 1.00 | 44.18 | |
| ATOM | 427 | CA | PHE | | 211 | −32.382 | 102.749 | −63.255 | 1.00 | 46.44 | |
| ATOM | 428 | CB | PHE | | 211 | −31.700 | 103.856 | −64.063 | 1.00 | 47.76 | |
| ATOM | 429 | CG | PHE | | 211 | −32.607 | 105.030 | −64.370 | 1.00 | 48.19 | |
| ATOM | 430 | CD1 | PHE | | 211 | −33.765 | 104.861 | −65.118 | 1.00 | 47.07 | |
| ATOM | 431 | CD2 | PHE | | 211 | −32.311 | 106.298 | −63.880 | 1.00 | 47.92 | |
| ATOM | 432 | CE1 | PHE | | 211 | −34.612 | 105.933 | −65.370 | 1.00 | 45.67 | |
| ATOM | 433 | CE2 | PHE | | 211 | −33.158 | 107.378 | −64.129 | 1.00 | 46.63 | |
| ATOM | 434 | CZ | PHE | | 211 | −34.305 | 107.189 | −64.874 | 1.00 | 45.23 | |
| ATOM | 435 | C | PHE | | 211 | −31.352 | 101.730 | −62.782 | 1.00 | 46.35 | |
| ATOM | 436 | O | PHE | | 211 | −30.536 | 101.294 | −63.570 | 1.00 | 45.82 | |
| ATOM | 437 | N | LEU | | 212 | −31.376 | 101.366 | −61.502 | 1.00 | 47.14 | |
| ATOM | 438 | CA | LEU | | 212 | −30.456 | 100.355 | −60.986 | 1.00 | 46.76 | |
| ATOM | 439 | CB | LEU | | 212 | −29.932 | 100.715 | −59.600 | 1.00 | 46.32 | |
| ATOM | 440 | CG | LEU | | 212 | −28.978 | 101.901 | −59.401 | 1.00 | 48.02 | |
| ATOM | 441 | CD1 | LEU | | 212 | −28.542 | 101.926 | −57.944 | 1.00 | 46.97 | |
| ATOM | 442 | CD2 | LEU | | 212 | −27.753 | 101.797 | −60.292 | 1.00 | 44.88 | |
| ATOM | 443 | C | LEU | | 212 | −31.300 | 99.098 | −60.887 | 1.00 | 47.83 | |
| ATOM | 444 | O | LEU | | 212 | −32.479 | 99.175 | −60.560 | 1.00 | 48.46 | |
| ATOM | 445 | N | THR | | 213 | −30.721 | 97.952 | −61.214 | 1.00 | 48.26 | |
| ATOM | 446 | CA | THR | | 213 | −31.443 | 96.682 | −61.135 | 1.00 | 49.99 | |
| ATOM | 447 | CB | THR | | 213 | −30.578 | 95.530 | −61.691 | 1.00 | 51.36 | |
| ATOM | 448 | OG1 | THR | | 213 | −30.281 | 95.780 | −63.072 | 1.00 | 54.33 | |
| ATOM | 449 | CG2 | THR | | 213 | −31.299 | 94.201 | −61.560 | 1.00 | 50.71 | |
| ATOM | 450 | C | THR | | 213 | −31.830 | 96.335 | −59.683 | 1.00 | 49.59 | |
| ATOM | 451 | O | THR | | 213 | −30.996 | 96.357 | −58.769 | 1.00 | 47.56 | |
| ATOM | 452 | N | ALA | | 214 | −33.096 | 96.001 | −59.473 | 1.00 | 50.39 | |
| ATOM | 453 | CA | ALA | | 214 | −33.577 | 95.660 | −58.131 | 1.00 | 52.38 | |
| ATOM | 454 | CB | ALA | | 214 | −35.075 | 95.911 | −58.030 | 1.00 | 50.72 | |
| ATOM | 455 | C | ALA | | 214 | −33.289 | 94.222 | −57.723 | 1.00 | 51.99 | |
| ATOM | 456 | O | ALA | | 214 | −33.410 | 93.300 | −58.523 | 1.00 | 51.79 | |
| ATOM | 457 | N | LEU | | 215 | −32.915 | 94.043 | −56.467 | 1.00 | 53.66 | |
| ATOM | 458 | CA | LEU | | 215 | −32.654 | 92.717 | −55.934 | 1.00 | 55.95 | |
| ATOM | 459 | CB | LEU | | 215 | −31.605 | 92.789 | −54.854 | 1.00 | 57.54 | |
| ATOM | 460 | CG | LEU | | 215 | −31.542 | 91.471 | −54.114 | 1.00 | 59.86 | |
| ATOM | 461 | CD1 | LEU | | 215 | −31.035 | 90.396 | −55.080 | 1.00 | 61.69 | |
| ATOM | 462 | CD2 | LEU | | 215 | −30.637 | 91.617 | −52.903 | 1.00 | 60.97 | |
| ATOM | 463 | C | LEU | | 215 | −33.936 | 92.232 | −55.304 | 1.00 | 57.19 | |
| ATOM | 464 | O | LEU | | 215 | −34.271 | 92.662 | −54.218 | 1.00 | 58.06 | |
| ATOM | 465 | N | LYS | | 216 | −34.650 | 91.341 | −55.974 | 1.00 | 60.03 | |
| ATOM | 466 | CA | LYS | | 216 | −35.920 | 90.832 | −55.456 | 1.00 | 63.02 | |
| ATOM | 467 | CB | LYS | | 216 | −36.669 | 90.102 | −56.577 | 1.00 | 63.60 | |
| ATOM | 468 | CG | LYS | | 216 | −38.104 | 89.755 | −56.260 | 1.00 | 67.22 | |
| ATOM | 469 | CD | LYS | | 216 | −38.818 | 90.910 | −55.545 | 1.00 | 70.35 | |
| ATOM | 470 | CE | LYS | | 216 | −40.336 | 90.820 | −55.730 | 1.00 | 72.53 | |
| ATOM | 471 | NZ | LYS | | 216 | −40.855 | 89.419 | −55.593 | 1.00 | 75.06 | |
| ATOM | 472 | C | LYS | | 216 | −35.789 | 89.932 | −54.209 | 1.00 | 64.87 | |
| ATOM | 473 | O | LYS | | 216 | −36.374 | 90.244 | −53.175 | 1.00 | 65.85 | |
| ATOM | 474 | N | TYR | | 217 | −35.041 | 88.827 | −54.303 | 1.00 | 66.23 | |
| ATOM | 475 | CA | TYR | | 217 | −34.836 | 87.915 | −53.162 | 1.00 | 66.97 | |
| ATOM | 476 | CB | TYR | | 217 | −35.464 | 86.540 | −53.382 | 1.00 | 67.30 | |
| ATOM | 477 | CG | TYR | | 217 | −36.864 | 86.505 | −53.916 | 1.00 | 70.63 | |
| ATOM | 478 | CD1 | TYR | | 217 | −37.221 | 85.555 | −54.871 | 1.00 | 71.10 | |
| ATOM | 479 | CE1 | TYR | | 217 | −38.507 | 85.493 | −55.377 | 1.00 | 73.55 | |
| ATOM | 480 | CD2 | TYR | | 217 | −37.839 | 87.400 | −53.469 | 1.00 | 71.96 | |
| ATOM | 481 | CE2 | TYR | | 217 | −39.142 | 87.349 | −53.972 | 1.00 | 73.20 | |
| ATOM | 482 | CZ | TYR | | 217 | −39.464 | 86.392 | −54.931 | 1.00 | 74.31 | |
| ATOM | 483 | OH | TYR | | 217 | −40.725 | 86.350 | −55.479 | 1.00 | 74.98 | |
| ATOM | 484 | C | TYR | | 217 | −33.356 | 87.635 | −52.974 | 1.00 | 67.49 | |
| ATOM | 485 | O | TYR | | 217 | −32.584 | 87.654 | −53.928 | 1.00 | 68.35 | |
| ATOM | 486 | N | ALA | | 218 | −32.970 | 87.332 | −51.743 | 1.00 | 67.82 | |
| ATOM | 487 | CA | ALA | | 218 | −31.586 | 86.982 | −51.450 | 1.00 | 68.73 | |
| ATOM | 488 | CB | ALA | | 218 | −30.854 | 88.166 | −50.830 | 1.00 | 68.56 | |
| ATOM | 489 | C | ALA | | 218 | −31.573 | 85.783 | −50.494 | 1.00 | 68.25 | |
| ATOM | 490 | O | ALA | | 218 | −32.059 | 85.874 | −49.366 | 1.00 | 68.81 | |
| ATOM | 491 | N | PHE | | 219 | −31.043 | 84.654 | −50.954 | 1.00 | 67.51 | |
| ATOM | 492 | CA | PHE | | 219 | −30.969 | 83.470 | −50.116 | 1.00 | 66.91 | |
| ATOM | 493 | CB | PHE | | 219 | −32.091 | 82.485 | −50.454 | 1.00 | 66.69 | |
| ATOM | 494 | CG | PHE | | 219 | −31.932 | 81.783 | −51.783 | 1.00 | 66.57 | |
| ATOM | 495 | CD1 | PHE | | 219 | −32.509 | 82.302 | −52.936 | 1.00 | 66.88 | |
| ATOM | 496 | CD2 | PHE | | 219 | −31.254 | 80.568 | −51.870 | 1.00 | 67.08 | |
| ATOM | 497 | CE1 | PHE | | 219 | −32.422 | 81.617 | −54.161 | 1.00 | 67.21 | |
| ATOM | 498 | CE2 | PHE | | 219 | −31.162 | 79.881 | −53.084 | 1.00 | 66.87 | |
| ATOM | 499 | CZ | PHE | | 219 | −31.752 | 80.409 | −54.233 | 1.00 | 66.88 | |
| ATOM | 500 | C | PHE | | 219 | −29.630 | 82.766 | −50.242 | 1.00 | 68.12 | |
| ATOM | 501 | O | PHE | | 219 | −28.763 | 83.176 | −51.008 | 1.00 | 67.85 | |
| ATOM | 502 | N | GLN | | 220 | −29.462 | 81.697 | −49.480 | 1.00 | 69.46 | |
| ATOM | 503 | CA | GLN | | 220 | −28.224 | 80.944 | −49.542 | 1.00 | 70.77 | |
| ATOM | 504 | CB | GLN | | 220 | −27.263 | 81.403 | −48.448 | 1.00 | 72.88 | |
| ATOM | 505 | CG | GLN | | 220 | −27.842 | 81.326 | −47.058 | 1.00 | 76.22 | |
| ATOM | 506 | CD | GLN | | 220 | −26.798 | 80.964 | −46.013 | 1.00 | 78.98 | |
| ATOM | 507 | OE1 | GLN | | 220 | −25.706 | 81.555 | −45.958 | 1.00 | 79.29 | |
| ATOM | 508 | NE2 | GLN | | 220 | −27.131 | 79.989 | −45.167 | 1.00 | 79.95 | |
| ATOM | 509 | C | GLN | | 220 | −28.425 | 79.441 | −49.419 | 1.00 | 69.62 | |
| ATOM | 510 | O | GLN | | 220 | −29.275 | 78.970 | −48.666 | 1.00 | 69.23 | |
| ATOM | 511 | N | THR | | 221 | −27.630 | 78.703 | −50.182 | 1.00 | 67.81 | |
| ATOM | 512 | CA | THR | | 221 | −27.661 | 77.263 | −50.141 | 1.00 | 66.82 | |
| ATOM | 513 | CB | THR | | 221 | −27.335 | 76.637 | −51.513 | 1.00 | 66.59 | |
| ATOM | 514 | OG1 | THR | | 221 | −26.037 | 77.065 | −51.946 | 1.00 | 65.71 | |
| ATOM | 515 | CG2 | THR | | 221 | −28.374 | 77.037 | −52.541 | 1.00 | 65.82 | |
| ATOM | 516 | C | THR | | 221 | −26.565 | 76.895 | −49.153 | 1.00 | 67.45 | |
| ATOM | 517 | O | THR | | 221 | −26.013 | 77.751 | −48.466 | 1.00 | 67.42 | |
| ATOM | 518 | N | HIS | | 222 | −26.237 | 75.618 | −49.084 | 1.00 | 68.27 | |
| ATOM | 519 | CA | HIS | | 222 | −25.211 | 75.173 | −48.159 | 1.00 | 68.63 | |
| ATOM | 520 | CB | HIS | | 222 | −25.294 | 73.646 | −48.033 | 1.00 | 67.27 | |
| ATOM | 521 | CG | HIS | | 222 | −24.006 | 72.994 | −47.649 | 1.00 | 65.84 | |
| ATOM | 522 | CD2 | HIS | | 222 | −23.502 | 72.654 | −46.439 | 1.00 | 63.40 | |
| ATOM | 523 | ND1 | HIS | | 222 | −23.053 | 72.637 | −48.579 | 1.00 | 64.42 | |
| ATOM | 524 | CE1 | HIS | | 222 | −22.016 | 72.103 | −47.959 | 1.00 | 64.14 | |
| ATOM | 525 | NE2 | HIS | | 222 | −22.263 | 72.102 | −46.659 | 1.00 | 64.39 | |
| ATOM | 526 | C | HIS | | 222 | −23.792 | 75.633 | −48.536 | 1.00 | 68.55 | |
| ATOM | 527 | O | HIS | | 222 | −22.905 | 75.669 | −47.686 | 1.00 | 88.63 | |
| ATOM | 528 | N | ASP | | 223 | −23.572 | 76.008 | −49.792 | 1.00 | 68.37 | |
| ATOM | 529 | CA | ASP | | 223 | −22.235 | 76.440 | −50.186 | 1.00 | 68.77 | |
| ATOM | 530 | CB | ASP | | 223 | −21.414 | 75.226 | −50.629 | 1.00 | 70.58 | |
| ATOM | 531 | CG | ASP | | 223 | −21.555 | 74.931 | −52.114 | 1.00 | 73.04 | |
| ATOM | 532 | OD1 | ASP | | 223 | −22.658 | 75.130 | −52.673 | 1.00 | 75.26 | |
| ATOM | 533 | OD2 | ASP | | 223 | −20.561 | 74.490 | −52.726 | 1.00 | 74.49 | |
| ATOM | 534 | C | ASP | | 223 | −22.213 | 77.503 | −51.289 | 1.00 | 67.78 | |
| ATOM | 535 | O | ASP | | 223 | −21.252 | 77.591 | −52.054 | 1.00 | 67.27 | |
| ATOM | 536 | N | ARG | | 224 | −23.265 | 78.308 | −51.371 | 1.00 | 66.12 | |
| ATOM | 537 | CA | ARG | | 224 | −23.338 | 79.344 | −52.390 | 1.00 | 64.62 | |
| ATOM | 538 | CB | ARG | | 224 | −23.817 | 78.756 | −53.727 | 1.00 | 64.01 | |
| ATOM | 539 | CG | ARG | | 224 | −22.984 | 77.588 | −54.244 | 1.00 | 63.37 | |
| ATOM | 540 | CD | ARG | | 224 | −23.124 | 77.399 | −55.734 | 1.00 | 64.16 | |
| ATOM | 541 | NE | ARG | | 224 | −22.335 | 76.268 | −56.207 | 1.00 | 65.27 | |
| ATOM | 542 | CZ | ARG | | 224 | −22.020 | 76.057 | −57.482 | 1.00 | 68.32 | |
| ATOM | 543 | NH1 | ARG | | 224 | −22.421 | 76.903 | −58.425 | 1.00 | 68.36 | |
| ATOM | 544 | NH2 | ARG | | 224 | −21.301 | 74.995 | −57.823 | 1.00 | 69.71 | |
| ATOM | 545 | C | ARG | | 224 | −24.286 | 80.450 | −51.969 | 1.00 | 64.24 | |
| ATOM | 546 | O | ARG | | 224 | −25.214 | 80.227 | −51.189 | 1.00 | 65.02 | |
| ATOM | 547 | N | LEU | | 225 | −24.043 | 81.655 | −52.472 | 1.00 | 63.20 | |
| ATOM | 548 | CA | LEU | | 225 | −24.921 | 82.779 | −52.172 | 1.00 | 62.23 | |
| ATOM | 549 | CB | LEU | | 225 | −24.102 | 84.001 | −51.753 | 1.00 | 61.58 | |
| ATOM | 550 | CG | LEU | | 225 | −23.503 | 83.911 | −50.345 | 1.00 | 62.50 | |
| ATOM | 551 | CD1 | LEU | | 225 | −22.685 | 85.158 | −50.037 | 1.00 | 59.60 | |
| ATOM | 552 | CD2 | LEU | | 225 | −24.630 | 83.739 | −49.329 | 1.00 | 60.22 | |
| ATOM | 553 | C | LEU | | 225 | −25.755 | 83.075 | −53.424 | 1.00 | 61.43 | |
| ATOM | 554 | O | LEU | | 225 | −25.226 | 83.122 | −54.532 | 1.00 | 61.26 | |
| ATOM | 555 | N | CYS | | 226 | −27.060 | 83.252 | −53.246 | 1.00 | 60.03 | |
| ATOM | 556 | CA | CYS | | 226 | −27.944 | 83.522 | −54.371 | 1.00 | 58.96 | |
| ATOM | 557 | CB | CYS | | 226 | −28.967 | 82.405 | −54.509 | 1.00 | 57.08 | |
| ATOM | 558 | SG | CYS | | 226 | −28.212 | 80.855 | −54.887 | 1.00 | 58.85 | |
| ATOM | 559 | C | CYS | | 226 | −28.686 | 84.839 | −54.277 | 1.00 | 58.68 | |
| ATOM | 560 | O | CYS | | 226 | −29.344 | 85.120 | −53.276 | 1.00 | 58.96 | |
| ATOM | 561 | N | PHE | | 227 | −28.586 | 85.641 | −55.332 | 1.00 | 58.33 | |
| ATOM | 562 | CA | PHE | | 227 | −29.286 | 86.920 | −55.387 | 1.00 | 57.69 | |
| ATOM | 563 | CB | PHE | | 227 | −28.287 | 88.079 | −55.422 | 1.00 | 58.43 | |
| ATOM | 564 | CG | PHE | | 227 | −27.312 | 88.061 | −54.284 | 1.00 | 58.45 | |
| ATOM | 565 | CD1 | PHE | | 227 | −26.098 | 87.394 | −54.406 | 1.00 | 58.64 | |
| ATOM | 566 | CD2 | PHE | | 227 | −27.633 | 88.661 | −53.071 | 1.00 | 57.57 | |
| ATOM | 567 | CE1 | PHE | | 227 | −25.218 | 87.323 | −53.335 | 1.00 | 59.42 | |
| ATOM | 568 | CE2 | PHE | | 227 | −26.765 | 88.595 | −51.992 | 1.00 | 58.69 | |
| ATOM | 569 | CZ | PHE | | 227 | −25.554 | 87.926 | −52.120 | 1.00 | 59.88 | |
| ATOM | 570 | C | PHE | | 227 | −30.169 | 86.933 | −56.623 | 1.00 | 57.25 | |
| ATOM | 571 | O | PHE | | 227 | −29.690 | 87.051 | −57.747 | 1.00 | 57.19 | |
| ATOM | 572 | N | VAL | | 228 | −31.467 | 86.769 | −56.413 | 1.00 | 57.99 | |
| ATOM | 573 | CA | VAL | | 228 | −32.412 | 86.756 | −57.520 | 1.00 | 58.10 | |
| ATOM | 574 | CB | VAL | | 228 | −33.701 | 86.055 | −57.114 | 1.00 | 57.27 | |
| ATOM | 575 | CG1 | VAL | | 228 | −34.648 | 85.996 | −58.278 | 1.00 | 55.74 | |
| ATOM | 576 | CG2 | VAL | | 228 | −33.380 | 84.675 | −56.586 | 1.00 | 56.18 | |
| ATOM | 577 | C | VAL | | 228 | −32.715 | 88.198 | −57.855 | 1.00 | 60.10 | |
| ATOM | 578 | O | VAL | | 228 | −33.132 | 88.966 | −56.990 | 1.00 | 60.07 | |
| ATOM | 579 | N | MET | | 229 | −32.503 | 88.586 | −59.105 | 1.00 | 61.45 | |
| ATOM | 580 | CA | MET | | 229 | −32.771 | 89.968 | −59.452 | 1.00 | 62.54 | |
| ATOM | 581 | CB | MET | | 229 | −31.461 | 90.721 | −59.549 | 1.00 | 65.03 | |
| ATOM | 582 | CG | MET | | 229 | −30.643 | 90.590 | −58.306 | 1.00 | 67.47 | |
| ATOM | 583 | SD | MET | | 229 | −29.164 | 91.540 | −58.504 | 1.00 | 76.13 | |
| ATOM | 584 | CE | MET | | 229 | −28.364 | 90.629 | −59.918 | 1.00 | 71.82 | |
| ATOM | 585 | C | MET | | 229 | −33.614 | 90.215 | −60.689 | 1.00 | 62.39 | |
| ATOM | 586 | O | MET | | 229 | −33.666 | 89.405 | −61.608 | 1.00 | 59.76 | |
| ATOM | 587 | N | GLU | | 230 | −34.295 | 91.356 | −60.672 | 1.00 | 64.12 | |
| ATOM | 588 | CA | GLU | | 230 | −35.166 | 91.784 | −61.757 | 1.00 | 65.04 | |
| ATOM | 589 | CB | GLU | | 230 | −35.790 | 93.141 | −61.414 | 1.00 | 67.87 | |
| ATOM | 590 | CG | GLU | | 230 | −34.762 | 94.247 | −61.183 | 1.00 | 70.27 | |
| ATOM | 591 | CD | GLU | | 230 | −35.252 | 95.636 | −61.612 | 1.00 | 72.06 | |
| ATOM | 592 | OE1 | GLU | | 230 | −35.747 | 95.779 | −62.757 | 1.00 | 71.89 | |
| ATOM | 593 | OE2 | GLU | | 230 | −35.121 | 96.590 | −60.813 | 1.00 | 71.80 | |
| ATOM | 594 | C | GLU | | 230 | −34.408 | 91.886 | −63.083 | 1.00 | 64.01 | |
| ATOM | 595 | O | GLU | | 230 | −33.374 | 92.564 | −63.180 | 1.00 | 61.86 | |
| ATOM | 596 | N | TYR | | 231 | −34.946 | 91.214 | −64.099 | 1.00 | 62.96 | |
| ATOM | 597 | CA | TYR | | 231 | −34.344 | 91.192 | −65.423 | 1.00 | 62.88 | |
| ATOM | 598 | CB | TYR | | 231 | −34.292 | 89.748 | −65.939 | 1.00 | 64.32 | |
| ATOM | 599 | CG | TYR | | 231 | −33.449 | 89.561 | −67.178 | 1.00 | 65.66 | |
| ATOM | 600 | CD1 | TYR | | 231 | −32.097 | 89.907 | −67.186 | 1.00 | 66.23 | |
| ATOM | 601 | CE1 | TYR | | 231 | −31.309 | 89.729 | −68.331 | 1.00 | 66.82 | |
| ATOM | 602 | CD2 | TYR | | 231 | −33.998 | 89.033 | −68.340 | 1.00 | 66.65 | |
| ATOM | 603 | CE2 | TYR | | 231 | −33.224 | 88.850 | −69.485 | 1.00 | 67.80 | |
| ATOM | 604 | CZ | TYR | | 231 | −31.882 | 89.201 | −69.480 | 1.00 | 68.02 | |
| ATOM | 605 | OH | TYR | | 231 | −31.133 | 89.040 | −70.634 | 1.00 | 69.12 | |
| ATOM | 606 | C | TYR | | 231 | −35.099 | 92.070 | −66.428 | 1.00 | 61.93 | |
| ATOM | 607 | O | TYR | | 231 | −36.259 | 91.807 | −66.768 | 1.00 | 61.07 | |
| ATOM | 608 | N | ALA | | 232 | −34.424 | 93.119 | −66.886 | 1.00 | 60.67 | |
| ATOM | 609 | CA | ALA | | 232 | −34.978 | 94.040 | −67.868 | 1.00 | 59.12 | |
| ATOM | 610 | CB | ALA | | 232 | −34.019 | 95.188 | −68.095 | 1.00 | 55.69 | |
| ATOM | 611 | C | ALA | | 232 | −35.137 | 93.239 | −69.142 | 1.00 | 59.78 | |
| ATOM | 612 | O | ALA | | 232 | −34.228 | 92.518 | −69.539 | 1.00 | 60.55 | |
| ATOM | 613 | N | ASN | | 233 | −36.282 | 93.351 | −69.795 | 1.00 | 60.97 | |
| ATOM | 614 | CA | ASN | | 233 | −36.480 | 92.577 | −71.011 | 1.00 | 62.16 | |
| ATOM | 615 | CB | ASN | | 233 | −37.747 | 91.738 | −70.891 | 1.00 | 62.76 | |
| ATOM | 616 | CG | ASN | | 233 | −38.967 | 92.585 | −70.651 | 1.00 | 64.97 | |
| ATOM | 617 | OD1 | ASN | | 233 | −39.462 | 93.241 | −71.560 | 1.00 | 65.94 | |
| ATOM | 618 | ND2 | ASN | | 233 | −39.449 | 92.595 | −69.416 | 1.00 | 66.63 | |
| ATOM | 619 | C | ASN | | 233 | −36.516 | 93.423 | −72.276 | 1.00 | 61.44 | |
| ATOM | 620 | O | ASN | | 233 | −37.156 | 93.057 | −73.256 | 1.00 | 62.23 | |
| ATOM | 621 | N | GLY | | 234 | −35.833 | 94.559 | −72.252 | 1.00 | 60.69 | |
| ATOM | 622 | CA | GLY | | 234 | −35.772 | 95.391 | −73.437 | 1.00 | 60.43 | |
| ATOM | 623 | C | GLY | | 234 | −34.464 | 95.150 | −74.188 | 1.00 | 59.89 | |
| ATOM | 624 | O | GLY | | 234 | −34.115 | 95.916 | −75.087 | 1.00 | 60.92 | |
| ATOM | 625 | N | GLY | | 235 | −33.739 | 94.086 | −73.836 | 1.00 | 58.89 | |
| ATOM | 626 | CA | GLY | | 235 | −32.479 | 93.791 | −74.499 | 1.00 | 57.90 | |
| ATOM | 627 | C | GLY | | 235 | −31.341 | 94.707 | −74.058 | 1.00 | 58.49 | |
| ATOM | 628 | O | GLY | | 235 | −31.579 | 95.820 | −73.565 | 1.00 | 57.13 | |
| ATOM | 629 | N | GLU | | 236 | −30.097 | 94.263 | −74.246 | 1.00 | 57.67 | |
| ATOM | 630 | CA | GLU | | 236 | −28.964 | 95.069 | −73.818 | 1.00 | 57.69 | |
| ATOM | 631 | CB | GLU | | 236 | −27.746 | 94.193 | −73.452 | 1.00 | 59.66 | |
| ATOM | 632 | CG | GLU | | 236 | −27.375 | 93.077 | −74.411 | 1.00 | 63.88 | |
| ATOM | 633 | CD | GLU | | 236 | −28.072 | 91.754 | −74.100 | 1.00 | 66.30 | |
| ATOM | 634 | OE1 | GLU | | 236 | −29.250 | 91.600 | −74.489 | 1.00 | 68.95 | |
| ATOM | 635 | OE2 | GLU | | 236 | −27.444 | 90.872 | −73.464 | 1.00 | 66.50 | |
| ATOM | 636 | C | GLU | | 236 | −28.561 | 96.142 | −74.803 | 1.00 | 55.83 | |
| ATOM | 637 | O | GLU | | 236 | −28.650 | 95.964 | −76.006 | 1.00 | 54.26 | |
| ATOM | 638 | N | LEU | | 237 | −28.137 | 97.276 | −74.265 | 1.00 | 56.29 | |
| ATOM | 639 | CA | LEU | | 237 | −27.723 | 98.394 | −75.087 | 1.00 | 58.37 | |
| ATOM | 640 | CB | LEU | | 237 | −27.106 | 99.494 | −74.235 | 1.00 | 58.34 | |
| ATOM | 641 | CG | LEU | | 237 | −28.152 | 100.550 | −73.958 | 1.00 | 59.37 | |
| ATOM | 642 | CD1 | LEU | | 237 | −27.529 | 101.709 | −73.212 | 1.00 | 60.88 | |
| ATOM | 643 | CD2 | LEU | | 237 | −28.739 | 101.003 | −75.284 | 1.00 | 60.06 | |
| ATOM | 644 | C | LEU | | 237 | −26.744 | 97.979 | −76.157 | 1.00 | 58.43 | |
| ATOM | 645 | O | LEU | | 237 | −26.962 | 98.271 | −77.331 | 1.00 | 57.83 | |
| ATOM | 646 | N | PHE | | 238 | −25.666 | 97.309 | −75.752 | 1.00 | 60.01 | |
| ATOM | 647 | CA | PHE | | 238 | −24.662 | 96.843 | −76.700 | 1.00 | 61.53 | |
| ATOM | 648 | CB | PHE | | 238 | −23.785 | 95.771 | −76.066 | 1.00 | 65.08 | |
| ATOM | 649 | CG | PHE | | 238 | −23.085 | 94.932 | −77.064 | 1.00 | 69.50 | |
| ATOM | 650 | CD1 | PHE | | 238 | −22.148 | 95.501 | −77.926 | 1.00 | 72.18 | |
| ATOM | 651 | CD2 | PHE | | 238 | −23.418 | 93.592 | −77.212 | 1.00 | 71.82 | |
| ATOM | 652 | CE1 | PHE | | 238 | −21.548 | 94.754 | −78.933 | 1.00 | 73.89 | |
| ATOM | 653 | CE2 | PHE | | 238 | −22.828 | 92.821 | −78.215 | 1.00 | 75.14 | |
| ATOM | 654 | CZ | PHE | | 238 | −21.887 | 93.406 | −79.083 | 1.00 | 76.46 | |
| ATOM | 655 | C | PHE | | 238 | −25.337 | 96.271 | −77.957 | 1.00 | 60.98 | |
| ATOM | 656 | O | PHE | | 238 | −24.963 | 96.604 | −79.088 | 1.00 | 60.58 | |
| ATOM | 657 | N | PHE | | 239 | −26.328 | 95.408 | −77.754 | 1.00 | 58.82 | |
| ATOM | 658 | CA | PHE | | 239 | −27.048 | 94.832 | −78.873 | 1.00 | 59.06 | |
| ATOM | 659 | CB | PHE | | 239 | −28.184 | 93.935 | −78.357 | 1.00 | 61.11 | |
| ATOM | 660 | CG | PHE | | 239 | −29.231 | 93.605 | −79.394 | 1.00 | 60.03 | |
| ATOM | 661 | CD1 | PHE | | 239 | −29.021 | 92.591 | −80.318 | 1.00 | 60.34 | |
| ATOM | 662 | CD2 | PHE | | 239 | −30.408 | 94.349 | −79.469 | 1.00 | 59.71 | |
| ATOM | 663 | CE1 | PHE | | 239 | −29.963 | 92.324 | −81.315 | 1.00 | 60.57 | |
| ATOM | 664 | CE2 | PHE | | 239 | −31.357 | 94.095 | −80.456 | 1.00 | 59.34 | |
| ATOM | 665 | CZ | PHE | | 239 | −31.135 | 93.079 | −81.385 | 1.00 | 60.69 | |
| ATOM | 666 | C | PHE | | 239 | −27.613 | 95.961 | −79.749 | 1.00 | 59.66 | |
| ATOM | 667 | O | PHE | | 239 | −27.362 | 95.989 | −80.959 | 1.00 | 59.72 | |
| ATOM | 668 | N | HIS | | 240 | −28.365 | 96.882 | −79.125 | 1.00 | 57.95 | |
| ATOM | 669 | CA | HIS | | 240 | −28.981 | 98.023 | −79.816 | 1.00 | 56.69 | |
| ATOM | 670 | CB | HIS | | 240 | −29.832 | 98.858 | −78.851 | 1.00 | 56.30 | |
| ATOM | 671 | CG | HIS | | 240 | −31.104 | 98.192 | −78.439 | 1.00 | 52.58 | |
| ATOM | 672 | CD2 | HIS | | 240 | −31.538 | 97.772 | −77.229 | 1.00 | 52.51 | |
| ATOM | 673 | ND1 | HIS | | 240 | −32.061 | 97.801 | −79.349 | 1.00 | 52.75 | |
| ATOM | 674 | CE1 | HIS | | 240 | −33.027 | 97.155 | −78.719 | 1.00 | 52.02 | |
| ATOM | 675 | NE2 | HIS | | 240 | −32.734 | 97.123 | −77.432 | 1.00 | 54.37 | |
| ATOM | 676 | C | HIS | | 240 | −27.977 | 98.951 | −80.489 | 1.00 | 56.52 | |
| ATOM | 677 | O | HIS | | 240 | −28.146 | 99.314 | −81.657 | 1.00 | 55.95 | |
| ATOM | 678 | N | LEU | | 241 | −26.942 | 99.346 | −79.759 | 1.00 | 56.06 | |
| ATOM | 679 | CA | LEU | | 241 | −25.937 | 100.226 | −80.333 | 1.00 | 57.24 | |
| ATOM | 680 | CB | LEU | | 241 | −24.851 | 100.540 | −79.315 | 1.00 | 54.85 | |
| ATOM | 681 | CG | LEU | | 241 | −23.814 | 101.485 | −79.922 | 1.00 | 53.60 | |
| ATOM | 682 | CD1 | LEU | | 241 | −24.461 | 102.838 | −80.219 | 1.00 | 54.00 | |
| ATOM | 683 | CD2 | LEU | | 241 | −22.644 | 101.641 | −78.980 | 1.00 | 53.68 | |
| ATOM | 684 | C | LEU | | 241 | −25.276 | 99.631 | −81.587 | 1.00 | 60.01 | |
| ATOM | 685 | O | LEU | | 241 | −25.051 | 100.340 | −82.579 | 1.00 | 59.83 | |
| ATOM | 686 | N | SER | | 242 | −24.950 | 98.340 | −81.544 | 1.00 | 61.36 | |
| ATOM | 687 | CA | SER | | 242 | −24.313 | 97.705 | −82.685 | 1.00 | 62.66 | |
| ATOM | 688 | CB | SER | | 242 | −23.945 | 96.264 | −82.350 | 1.00 | 63.23 | |
| ATOM | 689 | OG | SER | | 242 | −25.104 | 95.454 | −82.301 | 1.00 | 64.02 | |
| ATOM | 690 | C | SER | | 242 | −25.246 | 97.725 | −83.895 | 1.00 | 63.78 | |
| ATOM | 691 | O | SER | | 242 | −24.793 | 97.840 | −85.030 | 1.00 | 63.77 | |
| ATOM | 692 | N | ARG | | 243 | −26.549 | 97.613 | −83.651 | 1.00 | 65.15 | |
| ATOM | 693 | CA | ARG | | 243 | −27.524 | 97.624 | −84.740 | 1.00 | 67.42 | |
| ATOM | 694 | CB | ARG | | 243 | −28.920 | 97.246 | −84.225 | 1.00 | 68.67 | |
| ATOM | 695 | CG | ARG | | 243 | −29.062 | 95.800 | −83.756 | 1.00 | 73.40 | |
| ATOM | 696 | CD | ARG | | 243 | −30.487 | 95.301 | −83.978 | 1.00 | 75.73 | |
| ATOM | 697 | NE | ARG | | 243 | −30.846 | 95.409 | −85.392 | 1.00 | 80.29 | |
| ATOM | 698 | CZ | ARG | | 243 | −31.959 | 94.928 | −85.944 | 1.00 | 81.51 | |
| ATOM | 699 | NH1 | ARG | | 243 | −32.857 | 94.289 | −85.201 | 1.00 | 83.68 | |
| ATOM | 700 | NH2 | ARG | | 243 | −32.169 | 95.081 | −87.249 | 1.00 | 79.94 | |
| ATOM | 701 | C | ARG | | 243 | −27.602 | 98.989 | −85.434 | 1.00 | 67.41 | |
| ATOM | 702 | O | ARG | | 243 | −27.903 | 99.091 | −86.625 | 1.00 | 69.08 | |
| ATOM | 703 | N | GLU | | 244 | −27.321 | 100.040 | −84.683 | 1.00 | 66.72 | |
| ATOM | 704 | CA | GLU | | 244 | −27.387 | 101.379 | −85.225 | 1.00 | 66.11 | |
| ATOM | 705 | CB | GLU | | 244 | −28.246 | 102.255 | −84.315 | 1.00 | 67.57 | |
| ATOM | 706 | CG | GLU | | 244 | −29.719 | 101.911 | −84.408 | 1.00 | 70.44 | |
| ATOM | 707 | CD | GLU | | 244 | −30.519 | 102.489 | −83.268 | 1.00 | 73.07 | |
| ATOM | 708 | OE1 | GLU | | 244 | −30.407 | 101.962 | −82.136 | 1.00 | 74.17 | |
| ATOM | 709 | OE2 | GLU | | 244 | −31.257 | 103.472 | −83.505 | 1.00 | 75.33 | |
| ATOM | 710 | C | GLU | | 244 | −26.036 | 102.020 | −85.428 | 1.00 | 64.24 | |
| ATOM | 711 | O | GLU | | 244 | −25.969 | 103.169 | −85.861 | 1.00 | 64.90 | |
| ATOM | 712 | N | ARG | | 245 | −24.971 | 101.280 | −85.133 | 1.00 | 61.38 | |
| ATOM | 713 | CA | ARG | | 245 | −23.615 | 101.792 | −85.283 | 1.00 | 60.26 | |
| ATOM | 714 | CB | ARG | | 245 | −23.356 | 102.182 | −86.744 | 1.00 | 60.57 | |
| ATOM | 715 | CG | ARG | | 245 | −22.008 | 102.841 | −86.994 | 1.00 | 62.54 | |
| ATOM | 716 | CD | ARG | | 245 | −20.823 | 101.908 | −86.739 | 1.00 | 66.16 | |
| ATOM | 717 | NE | ARG | | 245 | −20.294 | 101.255 | −87.946 | 1.00 | 69.11 | |
| ATOM | 718 | CZ | ARG | | 245 | −20.907 | 100.293 | −88.643 | 1.00 | 70.48 | |
| ATOM | 719 | NH1 | ARG | | 245 | −22.106 | 99.837 | −88.276 | 1.00 | 72.21 | |
| ATOM | 720 | NH2 | ARG | | 245 | −20.310 | 99.770 | −89.711 | 1.00 | 70.06 | |
| ATOM | 721 | C | ARG | | 245 | −23.358 | 102.986 | −84.358 | 1.00 | 58.76 | |
| ATOM | 722 | O | ARG | | 245 | −22.300 | 103.074 | −83.736 | 1.00 | 59.27 | |
| ATOM | 723 | N | VAL | | 246 | −24.327 | 103.898 | −84.273 | 1.00 | 56.88 | |
| ATOM | 724 | CA | VAL | | 246 | −24.231 | 105.079 | −83.425 | 1.00 | 56.30 | |
| ATOM | 725 | CB | VAL | | 246 | −23.514 | 106.234 | −84.128 | 1.00 | 56.75 | |
| ATOM | 726 | CG1 | VAL | | 246 | −23.012 | 107.211 | −83.110 | 1.00 | 55.87 | |
| ATOM | 727 | CG2 | VAL | | 246 | −22.368 | 105.728 | −84.938 | 1.00 | 58.09 | |
| ATOM | 728 | C | VAL | | 246 | −25.614 | 105.591 | −83.015 | 1.00 | 56.68 | |
| ATOM | 729 | O | VAL | | 246 | −26.624 | 105.216 | −83.599 | 1.00 | 56.87 | |
| ATOM | 730 | N | PHE | | 247 | −25.633 | 106.454 | −82.002 | 1.00 | 55.57 | |
| ATOM | 731 | CA | PHE | | 247 | −26.849 | 107.048 | −81.462 | 1.00 | 54.79 | |
| ATOM | 732 | CB | PHE | | 247 | −26.962 | 106.771 | −79.960 | 1.00 | 53.15 | |
| ATOM | 733 | CG | PHE | | 247 | −27.359 | 105.363 | −79.613 | 1.00 | 52.02 | |
| ATOM | 734 | CD1 | PHE | | 247 | −28.332 | 104.697 | −80.344 | 1.00 | 49.77 | |
| ATOM | 735 | CD2 | PHE | | 247 | −26.812 | 104.730 | −78.492 | 1.00 | 53.62 | |
| ATOM | 736 | CE1 | PHE | | 247 | −28.761 | 103.434 | −79.967 | 1.00 | 48.10 | |
| ATOM | 737 | CE2 | PHE | | 247 | −27.242 | 103.452 | −78.107 | 1.00 | 51.99 | |
| ATOM | 738 | CZ | PHE | | 247 | −28.219 | 102.809 | −78.849 | 1.00 | 50.19 | |
| ATOM | 739 | C | PHE | | 247 | −26.821 | 108.565 | −81.648 | 1.00 | 56.44 | |
| ATOM | 740 | O | PHE | | 247 | −25.744 | 109.163 | −81.763 | 1.00 | 55.30 | |
| ATOM | 741 | N | THR | | 248 | −28.000 | 109.186 | −81.645 | 1.00 | 56.67 | |
| ATOM | 742 | CA | THR | | 248 | −28.104 | 110.633 | −81.792 | 1.00 | 58.42 | |
| ATOM | 743 | CB | THR | | 248 | −29.547 | 111.063 | −81.957 | 1.00 | 62.03 | |
| ATOM | 744 | OG1 | THR | | 248 | −29.983 | 110.739 | −83.280 | 1.00 | 65.64 | |
| ATOM | 745 | CG2 | THR | | 248 | −29.701 | 112.564 | −81.699 | 1.00 | 64.81 | |
| ATOM | 746 | C | THR | | 248 | −27.595 | 111.270 | −80.535 | 1.00 | 57.20 | |
| ATOM | 747 | O | THR | | 248 | −27.885 | 110.783 | −79.457 | 1.00 | 58.47 | |
| ATOM | 748 | N | GLU | | 249 | −26.852 | 112.362 | −80.662 | 1.00 | 57.14 | |
| ATOM | 749 | CA | GLU | | 249 | −26.322 | 113.044 | −79.489 | 1.00 | 58.07 | |
| ATOM | 750 | CB | GLU | | 249 | −25.697 | 114.371 | −79.879 | 1.00 | 57.49 | |
| ATOM | 751 | CG | GLU | | 249 | −24.435 | 114.251 | −80.671 | 1.00 | 60.71 | |
| ATOM | 752 | CD | GLU | | 249 | −23.588 | 115.496 | −80.526 | 1.00 | 62.96 | |
| ATOM | 753 | OE1 | GLU | | 249 | −23.859 | 116.250 | −79.575 | 1.00 | 64.86 | |
| ATOM | 754 | OE2 | GLU | | 249 | −22.656 | 115.722 | −81.333 | 1.00 | 64.89 | |
| ATOM | 755 | C | GLU | | 249 | −27.405 | 113.283 | −78.435 | 1.00 | 58.76 | |
| ATOM | 756 | O | GLU | | 249 | −27.112 | 113.574 | −77.275 | 1.00 | 59.17 | |
| ATOM | 757 | N | GLU | | 250 | −28.659 | 113.184 | −78.848 | 1.00 | 58.27 | |
| ATOM | 758 | CA | GLU | | 250 | −29.751 | 113.358 | −77.921 | 1.00 | 58.77 | |
| ATOM | 759 | CB | GLU | | 250 | −31.032 | 113.731 | −78.660 | 1.00 | 62.56 | |
| ATOM | 760 | CG | GLU | | 250 | −31.430 | 115.197 | −78.537 | 1.00 | 62.89 | |
| ATOM | 761 | CD | GLU | | 250 | −31.662 | 115.600 | −77.106 | 1.00 | 62.79 | |
| ATOM | 762 | OE1 | GLU | | 250 | −30.666 | 115.956 | −76.439 | 1.00 | 62.66 | |
| ATOM | 763 | OE2 | GLU | | 250 | −32.830 | 115.534 | −76.657 | 1.00 | 62.48 | |
| ATOM | 764 | C | GLU | | 250 | −29.924 | 112.019 | −77.238 | 1.00 | 58.14 | |
| ATOM | 765 | O | GLU | | 250 | −30.025 | 111.947 | −76.002 | 1.00 | 58.42 | |
| ATOM | 766 | N | ARG | | 251 | −29.959 | 110.963 | −78.053 | 1.00 | 56.39 | |
| ATOM | 767 | CA | ARG | | 251 | −30.073 | 109.586 | −77.554 | 1.00 | 52.97 | |
| ATOM | 768 | CB | ARG | | 251 | −29.866 | 108.588 | −78.692 | 1.00 | 52.89 | |
| ATOM | 769 | CG | ARG | | 251 | −31.075 | 107.734 | −79.023 | 1.00 | 54.14 | |
| ATOM | 770 | CD | ARG | | 251 | −30.792 | 106.241 | −78.788 | 1.00 | 56.02 | |
| ATOM | 771 | NE | ARG | | 251 | −31.509 | 105.381 | −79.727 | 1.00 | 55.82 | |
| ATOM | 772 | CZ | ARG | | 251 | −31.772 | 104.094 | −79.528 | 1.00 | 56.02 | |
| ATOM | 773 | NH1 | ARG | | 251 | −31.386 | 103.496 | −78.418 | 1.00 | 56.87 | |
| ATOM | 774 | NH2 | ARG | | 251 | −32.420 | 103.398 | −80.445 | 1.00 | 59.05 | |
| ATOM | 775 | C | ARG | | 251 | −29.005 | 109.367 | −76.478 | 1.00 | 50.39 | |
| ATOM | 776 | O | ARG | | 251 | −29.300 | 109.000 | −75.341 | 1.00 | 47.70 | |
| ATOM | 777 | N | ALA | | 252 | −27.755 | 109.610 | −76.828 | 1.00 | 49.25 | |
| ATOM | 778 | CA | ALA | | 252 | −26.708 | 109.436 | −75.838 | 1.00 | 50.43 | |
| ATOM | 779 | CB | ALA | | 252 | −25.374 | 109.994 | −76.337 | 1.00 | 48.80 | |
| ATOM | 780 | C | ALA | | 252 | −27.215 | 110.236 | −74.651 | 1.00 | 50.42 | |
| ATOM | 781 | O | ALA | | 252 | −27.503 | 109.671 | −73.603 | 1.00 | 51.77 | |
| ATOM | 782 | N | ALA | | 253 | −27.351 | 111.550 | −74.834 | 1.00 | 50.34 | |
| ATOM | 783 | CA | ALA | | 253 | −27.850 | 112.436 | −73.783 | 1.00 | 47.84 | |
| ATOM | 784 | CB | ALA | | 253 | −28.379 | 113.721 | −74.395 | 1.00 | 49.11 | |
| ATOM | 785 | C | ALA | | 253 | −28.946 | 111.748 | −72.958 | 1.00 | 45.27 | |
| ATOM | 786 | O | ALA | | 253 | −28.860 | 111.687 | −71.739 | 1.00 | 44.68 | |
| ATOM | 787 | N | PHE | | 254 | −29.964 | 111.211 | −73.605 | 1.00 | 41.73 | |
| ATOM | 788 | CA | PHE | | 254 | −31.004 | 110.548 | −72.841 | 1.00 | 42.16 | |
| ATOM | 789 | CB | PHE | | 254 | −31.966 | 109.856 | −73.777 | 1.00 | 45.56 | |
| ATOM | 790 | CG | PHE | | 254 | −33.084 | 109.171 | −73.075 | 1.00 | 50.41 | |
| ATOM | 791 | CD1 | PHE | | 254 | −34.144 | 109.908 | −72.559 | 1.00 | 50.78 | |
| ATOM | 792 | CD2 | PHE | | 254 | −33.098 | 107.783 | −72.953 | 1.00 | 51.98 | |
| ATOM | 793 | CE1 | PHE | | 254 | −35.212 | 109.281 | −71.932 | 1.00 | 53.54 | |
| ATOM | 794 | CE2 | PHE | | 254 | −34.167 | 107.137 | −72.325 | 1.00 | 52.46 | |
| ATOM | 795 | CZ | PHE | | 254 | −35.228 | 107.890 | −71.816 | 1.00 | 53.65 | |
| ATOM | 796 | C | PHE | | 254 | −30.448 | 109.517 | −71.830 | 1.00 | 41.57 | |
| ATOM | 797 | O | PHE | | 254 | −30.562 | 109.717 | −70.618 | 1.00 | 41.69 | |
| ATOM | 798 | N | TYR | | 255 | −29.860 | 108.420 | −72.336 | 1.00 | 40.48 | |
| ATOM | 799 | CA | TYR | | 255 | −29.263 | 107.327 | −71.525 | 1.00 | 32.92 | |
| ATOM | 800 | CB | TYR | | 255 | −28.545 | 106.320 | −72.423 | 1.00 | 31.84 | |
| ATOM | 801 | CG | TYR | | 255 | −29.498 | 105.630 | −73.390 | 1.00 | 32.14 | |
| ATOM | 802 | CD1 | TYR | | 255 | −30.676 | 105.070 | −72.938 | 1.00 | 31.97 | |
| ATOM | 803 | CE1 | TYR | | 255 | −31.586 | 104.494 | −73.828 | 1.00 | 34.25 | |
| ATOM | 804 | CD2 | TYR | | 255 | −29.238 | 105.592 | −74.758 | 1.00 | 30.32 | |
| ATOM | 805 | CE2 | TYR | | 255 | −30.130 | 105.029 | −75.647 | 1.00 | 30.67 | |
| ATOM | 806 | CZ | TYR | | 255 | −31.309 | 104.484 | −75.180 | 1.00 | 33.24 | |
| ATOM | 807 | OH | TYR | | 255 | −32.236 | 103.968 | −76.058 | 1.00 | 31.95 | |
| ATOM | 808 | C | TYR | | 255 | −28.287 | 107.870 | −70.525 | 1.00 | 31.24 | |
| ATOM | 809 | O | TYR | | 255 | −28.308 | 107.513 | −69.350 | 1.00 | 31.52 | |
| ATOM | 810 | N | GLY | | 256 | −27.443 | 108.770 | −70.984 | 1.00 | 27.74 | |
| ATOM | 811 | CA | GLY | | 256 | −26.474 | 109.351 | −70.098 | 1.00 | 29.40 | |
| ATOM | 812 | C | GLY | | 256 | −27.112 | 109.949 | −68.868 | 1.00 | 34.13 | |
| ATOM | 813 | O | GLY | | 256 | −26.577 | 109.746 | −67.753 | 1.00 | 36.09 | |
| ATOM | 814 | N | ALA | | 257 | −28.241 | 110.670 | −69.048 | 1.00 | 33.48 | |
| ATOM | 815 | CA | ALA | | 257 | −28.923 | 111.323 | −67.933 | 1.00 | 32.60 | |
| ATOM | 816 | CB | ALA | | 257 | −30.041 | 112.328 | −68.450 | 1.00 | 34.66 | |
| ATOM | 817 | C | ALA | | 257 | −29.514 | 110.319 | −66.936 | 1.00 | 34.67 | |
| ATOM | 818 | O | ALA | | 257 | −29.375 | 110.514 | −65.726 | 1.00 | 32.71 | |
| ATOM | 819 | N | GLU | | 258 | −30.174 | 109.264 | −67.418 | 1.00 | 33.09 | |
| ATOM | 820 | CA | GLU | | 258 | −30.703 | 108.280 | −66.487 | 1.00 | 37.82 | |
| ATOM | 821 | CB | GLU | | 258 | −31.513 | 107.204 | −67.227 | 1.00 | 38.53 | |
| ATOM | 822 | CG | GLU | | 258 | −32.633 | 107.806 | −68.066 | 1.00 | 41.10 | |
| ATOM | 823 | CD | GLU | | 258 | −33.728 | 106.821 | −68.411 | 1.00 | 44.29 | |
| ATOM | 824 | OE1 | GLU | | 258 | −33.406 | 105.642 | −68.711 | 1.00 | 43.99 | |
| ATOM | 825 | OE2 | GLU | | 258 | −34.916 | 107.225 | −68.391 | 1.00 | 41.43 | |
| ATOM | 826 | C | GLU | | 258 | −29.516 | 107.661 | −65.717 | 1.00 | 40.77 | |
| ATOM | 827 | O | GLU | | 258 | −29.586 | 107.446 | −64.480 | 1.00 | 41.98 | |
| ATOM | 828 | N | ILE | | 259 | −28.410 | 107.427 | −66.427 | 1.00 | 39.37 | |
| ATOM | 829 | CA | ILE | | 259 | −27.246 | 106.862 | −65.769 | 1.00 | 41.44 | |
| ATOM | 830 | CB | ILE | | 259 | −26.061 | 106.538 | −66.743 | 1.00 | 40.57 | |
| ATOM | 831 | CG2 | ILE | | 259 | −24.909 | 105.968 | −65.969 | 1.00 | 37.79 | |
| ATOM | 832 | CG1 | ILE | | 259 | −26.493 | 105.531 | −67.800 | 1.00 | 40.69 | |
| ATOM | 833 | CD1 | ILE | | 259 | −25.437 | 105.291 | −68.852 | 1.00 | 45.72 | |
| ATOM | 834 | C | ILE | | 259 | −26.754 | 107.871 | −64.753 | 1.00 | 41.09 | |
| ATOM | 835 | O | ILE | | 259 | −26.461 | 107.494 | −63.614 | 1.00 | 42.53 | |
| ATOM | 836 | N | VAL | | 260 | −26.656 | 109.144 | −65.164 | 1.00 | 40.14 | |
| ATOM | 837 | CA | VAL | | 260 | −26.176 | 110.200 | −64.254 | 1.00 | 38.43 | |
| ATOM | 838 | CB | VAL | | 260 | −26.066 | 111.595 | −64.954 | 1.00 | 37.91 | |
| ATOM | 839 | CG1 | VAL | | 260 | −25.551 | 112.633 | −63.980 | 1.00 | 34.74 | |
| ATOM | 840 | CG2 | VAL | | 260 | −25.111 | 111.532 | −66.149 | 1.00 | 34.85 | |
| ATOM | 841 | C | VAL | | 260 | −27.100 | 110.305 | −63.039 | 1.00 | 38.63 | |
| ATOM | 842 | O | VAL | | 260 | −26.676 | 110.673 | −61.949 | 1.00 | 36.65 | |
| ATOM | 843 | N | SER | | 261 | −28.366 | 109.948 | −63.226 | 1.00 | 40.78 | |
| ATOM | 844 | CA | SER | | 261 | −29.316 | 109.992 | −62.128 | 1.00 | 42.94 | |
| ATOM | 845 | CB | SER | | 261 | −30.750 | 109.902 | −62.652 | 1.00 | 41.46 | |
| ATOM | 846 | OG | SER | | 261 | −31.672 | 109.806 | −61.591 | 1.00 | 38.11 | |
| ATOM | 847 | C | SER | | 261 | −29.023 | 108.826 | −61.183 | 1.00 | 43.11 | |
| ATOM | 848 | O | SER | | 261 | −29.071 | 108.965 | −59.963 | 1.00 | 43.59 | |
| ATOM | 849 | N | ALA | | 262 | −28.705 | 107.676 | −61.764 | 1.00 | 45.11 | |
| ATOM | 850 | CA | ALA | | 262 | −28.395 | 106.486 | −60.971 | 1.00 | 42.89 | |
| ATOM | 851 | CB | ALA | | 262 | −28.224 | 105.289 | −61.880 | 1.00 | 42.22 | |
| ATOM | 852 | C | ALA | | 262 | −27.152 | 106.697 | −60.117 | 1.00 | 42.66 | |
| ATOM | 853 | O | ALA | | 262 | −27.131 | 106.329 | −58.940 | 1.00 | 45.22 | |
| ATOM | 854 | N | LEU | | 263 | −26.129 | 107.317 | −60.680 | 1.00 | 41.70 | |
| ATOM | 855 | CA | LEU | | 263 | −24.910 | 107.534 | −59.919 | 1.00 | 42.13 | |
| ATOM | 856 | CB | LEU | | 263 | −23.708 | 107.744 | −60.833 | 1.00 | 40.05 | |
| ATOM | 857 | CG | LEU | | 263 | −23.493 | 106.752 | −61.964 | 1.00 | 41.42 | |
| ATOM | 858 | CD1 | LEU | | 263 | −22.215 | 107.141 | −62.723 | 1.00 | 39.22 | |
| ATOM | 859 | CD2 | LEU | | 263 | −23.411 | 105.349 | −61.393 | 1.00 | 40.79 | |
| ATOM | 860 | C | LEU | | 263 | −24.969 | 108.689 | −58.943 | 1.00 | 43.19 | |
| ATOM | 861 | O | LEU | | 263 | −24.247 | 108.669 | −57.954 | 1.00 | 45.28 | |
| ATOM | 862 | N | GLU | | 264 | −25.768 | 109.721 | −59.212 | 1.00 | 44.12 | |
| ATOM | 863 | CA | GLU | | 264 | −25.852 | 110.821 | −58.239 | 1.00 | 44.00 | |
| ATOM | 864 | CB | GLU | | 264 | −26.829 | 111.903 | −58.680 | 1.00 | 45.06 | |
| ATOM | 865 | CG | GLU | | 264 | −26.912 | 113.061 | −57.671 | 1.00 | 51.94 | |
| ATOM | 866 | CD | GLU | | 264 | −28.131 | 113.968 | −57.861 | 1.00 | 53.09 | |
| ATOM | 867 | OE1 | GLU | | 264 | −29.238 | 113.454 | −58.170 | 1.00 | 52.09 | |
| ATOM | 868 | OE2 | GLU | | 264 | −27.982 | 115.195 | −57.672 | 1.00 | 53.73 | |
| ATOM | 869 | C | GLU | | 264 | −26.382 | 110.190 | −56.953 | 1.00 | 42.94 | |
| ATOM | 870 | O | GLU | | 264 | −25.895 | 110.474 | −55.848 | 1.00 | 39.40 | |
| ATOM | 871 | N | TYR | | 265 | −27.372 | 109.307 | −57.136 | 1.00 | 43.31 | |
| ATOM | 872 | CA | TYR | | 265 | −28.005 | 108.608 | −56.041 | 1.00 | 43.11 | |
| ATOM | 873 | CB | TYR | | 265 | −29.120 | 107.687 | −56.543 | 1.00 | 47.99 | |
| ATOM | 874 | CG | TYR | | 265 | −30.016 | 107.222 | −55.405 | 1.00 | 55.41 | |
| ATOM | 875 | CD1 | TYR | | 265 | −30.944 | 108.110 | −54.826 | 1.00 | 58.93 | |
| ATOM | 876 | CE1 | TYR | | 265 | −31.756 | 107.729 | −53.757 | 1.00 | 59.78 | |
| ATOM | 877 | CD2 | TYR | | 265 | −29.925 | 105.928 | −54.878 | 1.00 | 56.06 | |
| ATOM | 878 | CE2 | TYR | | 265 | −30.736 | 105.527 | −53.803 | 1.00 | 59.29 | |
| ATOM | 879 | CZ | TYR | | 265 | −31.655 | 106.441 | −53.246 | 1.00 | 61.52 | |
| ATOM | 880 | OH | TYR | | 265 | −32.488 | 106.103 | −52.187 | 1.00 | 60.96 | |
| ATOM | 881 | C | TYR | | 265 | −27.009 | 107.763 | −55.254 | 1.00 | 41.53 | |
| ATOM | 882 | O | TYR | | 265 | −26.976 | 107.846 | −54.015 | 1.00 | 37.07 | |
| ATOM | 883 | N | LEU | | 266 | −26.224 | 106.951 | −55.986 | 1.00 | 40.89 | |
| ATOM | 884 | CA | LEU | | 266 | −25.225 | 106.047 | −55.395 | 1.00 | 38.93 | |
| ATOM | 885 | CB | LEU | | 266 | −24.625 | 105.101 | −56.444 | 1.00 | 37.40 | |
| ATOM | 886 | CG | LEU | | 266 | −25.576 | 103.988 | −56.927 | 1.00 | 36.00 | |
| ATOM | 887 | CD1 | LEU | | 266 | −25.211 | 103.561 | −58.332 | 1.00 | 36.05 | |
| ATOM | 888 | CD2 | LEU | | 266 | −25.551 | 102.810 | −55.979 | 1.00 | 34.11 | |
| ATOM | 889 | C | LEU | | 266 | −24.129 | 106.786 | −54.685 | 1.00 | 37.15 | |
| ATOM | 890 | O | LEU | | 266 | −23.849 | 106.508 | −53.550 | 1.00 | 35.44 | |
| ATOM | 891 | N | HIS | | 267 | −23.523 | 107.751 | −55.344 | 1.00 | 38.59 | |
| ATOM | 892 | CA | HIS | | 267 | −22.468 | 108.502 | −54.713 | 1.00 | 40.15 | |
| ATOM | 893 | CB | HIS | | 267 | −21.913 | 109.539 | −55.671 | 1.00 | 41.44 | |
| ATOM | 894 | CG | HIS | | 267 | −21.177 | 108.961 | −56.835 | 1.00 | 42.88 | |
| ATOM | 895 | CD2 | HIS | | 267 | −21.001 | 107.683 | −57.234 | 1.00 | 42.55 | |
| ATOM | 896 | ND1 | HIS | | 267 | −20.524 | 109.745 | −57.759 | 1.00 | 41.97 | |
| ATOM | 897 | CE1 | HIS | | 267 | −19.978 | 108.973 | −58.680 | 1.00 | 43.09 | |
| ATOM | 898 | NE2 | HIS | | 267 | −20.253 | 107.717 | −58.384 | 1.00 | 43.80 | |
| ATOM | 899 | C | HIS | | 267 | −22.974 | 109.212 | −53.478 | 1.00 | 43.29 | |
| ATOM | 900 | O | HIS | | 267 | −22.187 | 109.612 | −52.628 | 1.00 | 42.37 | |
| ATOM | 901 | N | SER | | 268 | −24.287 | 109.406 | −53.384 | 1.00 | 47.54 | |
| ATOM | 902 | CA | SER | | 268 | −24.829 | 110.110 | −52.232 | 1.00 | 51.53 | |
| ATOM | 903 | CB | SER | | 268 | −26.224 | 110.671 | −52.527 | 1.00 | 52.10 | |
| ATOM | 904 | OG | SER | | 268 | −27.199 | 109.637 | −52.556 | 1.00 | 59.22 | |
| ATOM | 905 | C | SER | | 268 | −24.871 | 109.149 | −51.062 | 1.00 | 51.38 | |
| ATOM | 906 | O | SER | | 268 | −24.747 | 109.553 | −49.916 | 1.00 | 52.88 | |
| ATOM | 907 | N | ARG | | 269 | −25.056 | 107.875 | −51.355 | 1.00 | 51.82 | |
| ATOM | 908 | CA | ARG | | 269 | −25.060 | 106.869 | −50.312 | 1.00 | 53.73 | |
| ATOM | 909 | CB | ARG | | 269 | −25.949 | 105.693 | −50.712 | 1.00 | 57.67 | |
| ATOM | 910 | CG | ARG | | 269 | −27.399 | 106.037 | −50.921 | 1.00 | 63.45 | |
| ATOM | 911 | CD | ARG | | 269 | −28.018 | 106.576 | −49.645 | 1.00 | 68.29 | |
| ATOM | 912 | NE | ARG | | 269 | −29.466 | 106.764 | −49.759 | 1.00 | 73.68 | |
| ATOM | 913 | CZ | ARG | | 269 | −30.064 | 107.657 | −50.552 | 1.00 | 75.36 | |
| ATOM | 914 | NH1 | ARG | | 269 | −29.349 | 108.472 | −51.326 | 1.00 | 77.14 | |
| ATOM | 915 | NH2 | ARG | | 269 | −31.389 | 107.741 | −50.569 | 1.00 | 75.25 | |
| ATOM | 916 | C | ARG | | 269 | −23.613 | 106.382 | −50.143 | 1.00 | 53.04 | |
| ATOM | 917 | O | ARG | | 269 | −23.389 | 105.247 | −49.745 | 1.00 | 51.54 | |
| ATOM | 918 | N | ASP | | 270 | −22.646 | 107.236 | −50.487 | 1.00 | 52.95 | |
| ATOM | 919 | CA | ASP | | 270 | −21.218 | 106.921 | −50.381 | 1.00 | 53.19 | |
| ATOM | 920 | CB | ASP | | 270 | −20.827 | 106.915 | −48.894 | 1.00 | 56.60 | |
| ATOM | 921 | CG | ASP | | 270 | −19.328 | 107.150 | −48.655 | 1.00 | 61.82 | |
| ATOM | 922 | OD1 | ASP | | 270 | −18.507 | 106.243 | −48.926 | 1.00 | 65.60 | |
| ATOM | 923 | OD2 | ASP | | 270 | −18.965 | 108.250 | −48.185 | 1.00 | 62.55 | |
| ATOM | 924 | C | ASP | | 270 | −20.845 | 105.572 | −51.051 | 1.00 | 52.23 | |
| ATOM | 925 | O | ASP | | 270 | −19.967 | 104.857 | −50.579 | 1.00 | 53.37 | |
| ATOM | 926 | N | VAL | | 271 | −21.500 | 105.246 | −52.164 | 1.00 | 48.97 | |
| ATOM | 927 | CA | VAL | | 271 | −21.282 | 103.999 | −52.896 | 1.00 | 46.17 | |
| ATOM | 928 | CB | VAL | | 271 | −22.597 | 103.308 | −53.150 | 1.00 | 45.22 | |
| ATOM | 929 | CG1 | VAL | | 271 | −22.411 | 102.187 | −54.152 | 1.00 | 46.54 | |
| ATOM | 930 | CG2 | VAL | | 271 | −23.158 | 102.816 | −51.868 | 1.00 | 46.65 | |
| ATOM | 931 | C | VAL | | 271 | −20.655 | 104.189 | −54.268 | 1.00 | 46.38 | |
| ATOM | 932 | O | VAL | | 271 | −21.259 | 104.830 | −55.118 | 1.00 | 47.00 | |
| ATOM | 933 | N | VAL | | 272 | −19.475 | 103.613 | −54.504 | 1.00 | 46.10 | |
| ATOM | 934 | CA | VAL | | 272 | −18.802 | 103.732 | −55.812 | 1.00 | 43.83 | |
| ATOM | 935 | CB | VAL | | 272 | −17.265 | 103.711 | −55.695 | 1.00 | 43.04 | |
| ATOM | 936 | CG1 | VAL | | 272 | −16.650 | 103.699 | −57.095 | 1.00 | 42.62 | |
| ATOM | 937 | CG2 | VAL | | 272 | −16.779 | 104.907 | −54.920 | 1.00 | 40.14 | |
| ATOM | 938 | C | VAL | | 272 | −19.195 | 102.568 | −56.701 | 1.00 | 42.40 | |
| ATOM | 939 | O | VAL | | 272 | −18.938 | 101.421 | −56.376 | 1.00 | 43.41 | |
| ATOM | 940 | N | TYR | | 273 | −19.790 | 102.842 | −57.844 | 1.00 | 40.51 | |
| ATOM | 941 | CA | TYR | | 273 | −20.202 | 101.732 | −58.678 | 1.00 | 37.57 | |
| ATOM | 942 | CB | TYR | | 273 | −21.125 | 102.229 | −59.766 | 1.00 | 32.36 | |
| ATOM | 943 | CG | TYR | | 273 | −22.007 | 101.116 | −60.224 | 1.00 | 28.72 | |
| ATOM | 944 | CD1 | TYR | | 273 | −22.905 | 100.536 | −59.354 | 1.00 | 28.66 | |
| ATOM | 945 | CE1 | TYR | | 273 | −23.703 | 99.472 | −59.756 | 1.00 | 29.61 | |
| ATOM | 946 | CD2 | TYR | | 273 | −21.917 | 100.613 | −61.511 | 1.00 | 27.83 | |
| ATOM | 947 | CE2 | TYR | | 273 | −22.714 | 99.550 | −61.923 | 1.00 | 29.06 | |
| ATOM | 948 | CZ | TYR | | 273 | −23.608 | 98.988 | −61.035 | 1.00 | 27.75 | |
| ATOM | 949 | OH | TYR | | 273 | −24.438 | 97.962 | −61.443 | 1.00 | 31.99 | |
| ATOM | 950 | C | TYR | | 273 | −19.148 | 100.794 | −59.311 | 1.00 | 36.59 | |
| ATOM | 951 | O | TYR | | 273 | −19.391 | 99.593 | −59.426 | 1.00 | 34.01 | |
| ATOM | 952 | N | ARG | | 274 | −18.032 | 101.347 | −59.771 | 1.00 | 37.36 | |
| ATOM | 953 | CA | ARG | | 274 | −16.952 | 100.573 | −60.400 | 1.00 | 41.86 | |
| ATOM | 954 | CB | ARG | | 274 | −16.509 | 99.420 | −59.490 | 1.00 | 42.11 | |
| ATOM | 955 | CG | ARG | | 274 | −15.915 | 99.829 | −58.157 | 1.00 | 44.64 | |
| ATOM | 956 | CD | ARG | | 274 | −15.293 | 98.607 | −57.485 | 1.00 | 47.09 | |
| ATOM | 957 | NE | ARG | | 274 | −14.693 | 98.910 | −56.188 | 1.00 | 48.75 | |
| ATOM | 958 | CZ | ARG | | 274 | −13.499 | 98.474 | −55.796 | 1.00 | 47.29 | |
| ATOM | 959 | NH1 | ARG | | 274 | −12.765 | 97.716 | −56.608 | 1.00 | 48.77 | |
| ATOM | 960 | NH2 | ARG | | 274 | −13.048 | 98.780 | −54.588 | 1.00 | 45.02 | |
| ATOM | 961 | C | ARG | | 274 | −17.244 | 99.968 | −61.769 | 1.00 | 43.19 | |
| ATOM | 962 | O | ARG | | 274 | −16.346 | 99.832 | −62.604 | 1.00 | 44.18 | |
| ATOM | 963 | N | ASP | | 275 | −18.498 | 99.594 | −61.993 | 1.00 | 45.10 | |
| ATOM | 964 | CA | ASP | | 275 | −18.877 | 98.934 | −63.231 | 1.00 | 43.85 | |
| ATOM | 965 | CB | ASP | | 275 | −19.548 | 97.603 | −62.910 | 1.00 | 48.48 | |
| ATOM | 966 | CG | ASP | | 275 | −18.578 | 96.578 | −62.322 | 1.00 | 54.54 | |
| ATOM | 967 | OD1 | ASP | | 275 | −17.969 | 96.835 | −61.252 | 1.00 | 53.37 | |
| ATOM | 968 | OD2 | ASP | | 275 | −18.433 | 95.493 | −62.939 | 1.00 | 60.03 | |
| ATOM | 969 | C | ASP | | 275 | −19.752 | 99.681 | −64.219 | 1.00 | 41.01 | |
| ATOM | 970 | O | ASP | | 275 | −20.620 | 99.074 | −64.844 | 1.00 | 40.47 | |
| ATOM | 971 | N | ILE | | 276 | −19.563 | 100.982 | −64.381 | 1.00 | 37.54 | |
| ATOM | 972 | CA | ILE | | 276 | −20.376 | 101.616 | −65.392 | 1.00 | 35.19 | |
| ATOM | 973 | CB | ILE | | 276 | −20.452 | 103.147 | −65.234 | 1.00 | 36.23 | |
| ATOM | 974 | CG2 | ILE | | 276 | −21.139 | 103.741 | −66.479 | 1.00 | 31.78 | |
| ATOM | 975 | CG1 | ILE | | 276 | −21.172 | 103.516 | −63.920 | 1.00 | 32.64 | |
| ATOM | 976 | CD1 | ILE | | 276 | −22.513 | 102.917 | −63.772 | 1.00 | 28.27 | |
| ATOM | 977 | C | ILE | | 276 | −19.757 | 101.302 | −66.764 | 1.00 | 34.89 | |
| ATOM | 978 | O | ILE | | 276 | −18.703 | 101.837 | −67.122 | 1.00 | 33.85 | |
| ATOM | 979 | N | LYS | | 277 | −20.405 | 100.442 | −67.538 | 1.00 | 33.22 | |
| ATOM | 980 | CA | LYS | | 277 | −19.865 | 100.132 | −68.833 | 1.00 | 37.67 | |
| ATOM | 981 | CB | LYS | | 277 | −18.602 | 99.230 | −68.694 | 1.00 | 40.12 | |
| ATOM | 982 | CG | LYS | | 277 | −18.758 | 97.959 | −67.894 | 1.00 | 40.43 | |
| ATOM | 983 | CD | LYS | | 277 | −17.397 | 97.537 | −67.358 | 1.00 | 49.81 | |
| ATOM | 984 | CE | LYS | | 277 | −17.280 | 96.033 | −67.084 | 1.00 | 49.65 | |
| ATOM | 985 | NZ | LYS | | 277 | −17.231 | 95.212 | −68.335 | 1.00 | 50.73 | |
| ATOM | 986 | C | LYS | | 277 | −20.945 | 99.493 | −69.652 | 1.00 | 37.96 | |
| ATOM | 987 | O | LYS | | 277 | −21.787 | 98.779 | −69.120 | 1.00 | 38.70 | |
| ATOM | 988 | N | LEU | | 278 | −20.892 | 99.745 | −70.956 | 1.00 | 41.01 | |
| ATOM | 989 | CA | LEU | | 278 | −21.901 | 99.300 | −71.921 | 1.00 | 43.27 | |
| ATOM | 990 | CB | LEU | | 278 | −21.344 | 99.464 | −73.341 | 1.00 | 45.84 | |
| ATOM | 991 | CG | LEU | | 278 | −22.252 | 99.148 | −74.535 | 1.00 | 47.04 | |
| ATOM | 992 | CD1 | LEU | | 278 | −23.423 | 100.103 | −74.582 | 1.00 | 47.73 | |
| ATOM | 993 | CD2 | LEU | | 278 | −21.430 | 99.245 | −75.820 | 1.00 | 49.67 | |
| ATOM | 994 | C | LEU | | 278 | −22.514 | 97.915 | −71.765 | 1.00 | 44.93 | |
| ATOM | 995 | O | LEU | | 278 | −23.717 | 97.712 | −72.009 | 1.00 | 43.40 | |
| ATOM | 996 | N | GLU | | 279 | −21.684 | 96.953 | −71.382 | 1.00 | 46.75 | |
| ATOM | 997 | CA | GLU | | 279 | −22.139 | 95.576 | −71.210 | 1.00 | 46.80 | |
| ATOM | 998 | CB | GLU | | 279 | −20.931 | 94.662 | −70.943 | 1.00 | 51.26 | |
| ATOM | 999 | CG | GLU | | 279 | −19.682 | 94.964 | −71.808 | 1.00 | 54.97 | |
| ATOM | 1000 | CD | GLU | | 279 | −19.043 | 96.309 | −71.458 | 1.00 | 60.05 | |
| ATOM | 1001 | OE1 | GLU | | 279 | −19.369 | 96.848 | −70.374 | 1.00 | 61.29 | |
| ATOM | 1002 | OE2 | GLU | | 279 | −18.214 | 96.826 | −72.250 | 1.00 | 63.32 | |
| ATOM | 1003 | C | GLU | | 279 | −23.131 | 95.509 | −70.056 | 1.00 | 44.42 | |
| ATOM | 1004 | O | GLU | | 279 | −24.081 | 94.751 | −70.100 | 1.00 | 44.41 | |
| ATOM | 1005 | N | ASN | | 280 | −22.906 | 96.318 | −69.027 | 1.00 | 42.54 | |
| ATOM | 1006 | CA | ASN | | 280 | −23.795 | 96.357 | −67.878 | 1.00 | 43.63 | |
| ATOM | 1007 | CB | ASN | | 280 | −23.067 | 96.910 | −66.635 | 1.00 | 44.99 | |
| ATOM | 1008 | CG | ASN | | 280 | −21.964 | 96.003 | −66.130 | 1.00 | 47.56 | |
| ATOM | 1009 | OD1 | ASN | | 280 | −21.175 | 95.489 | −66.896 | 1.00 | 52.55 | |
| ATOM | 1010 | ND2 | ASN | | 280 | −21.889 | 95.833 | −64.829 | 1.00 | 48.82 | |
| ATOM | 1011 | C | ASN | | 280 | −25.007 | 97.272 | −68.142 | 1.00 | 43.42 | |
| ATOM | 1012 | O | ASN | | 280 | −25.556 | 97.807 | −67.194 | 1.00 | 44.40 | |
| ATOM | 1013 | N | LEU | | 281 | −25.414 | 97.487 | −69.390 | 1.00 | 42.51 | |
| ATOM | 1014 | CA | LEU | | 281 | −26.565 | 98.375 | −69.637 | 1.00 | 42.52 | |
| ATOM | 1015 | CB | LEU | | 281 | −26.170 | 99.688 | −70.343 | 1.00 | 38.94 | |
| ATOM | 1016 | CG | LEU | | 281 | −25.149 | 100.642 | −69.685 | 1.00 | 40.96 | |
| ATOM | 1017 | CD1 | LEU | | 281 | −24.893 | 101.863 | −70.562 | 1.00 | 36.16 | |
| ATOM | 1018 | CD2 | LEU | | 281 | −25.652 | 101.071 | −68.330 | 1.00 | 39.50 | |
| ATOM | 1019 | C | LEU | | 281 | −27.634 | 97.710 | −70.465 | 1.00 | 43.42 | |
| ATOM | 1020 | O | LEU | | 281 | −27.381 | 97.229 | −71.563 | 1.00 | 43.54 | |
| ATOM | 1021 | N | MET | | 282 | −28.837 | 97.666 | −69.924 | 1.00 | 44.23 | |
| ATOM | 1022 | CA | MET | | 282 | −29.945 | 97.091 | −70.655 | 1.00 | 48.65 | |
| ATOM | 1023 | CB | MET | | 282 | −30.410 | 95.828 | −69.966 | 1.00 | 48.44 | |
| ATOM | 1024 | CG | MET | | 282 | −29.524 | 94.663 | −70.284 | 1.00 | 49.57 | |
| ATOM | 1025 | SD | MET | | 282 | −30.209 | 93.186 | −69.538 | 1.00 | 49.53 | |
| ATOM | 1026 | CE | MET | | 282 | −29.278 | 93.263 | −68.055 | 1.00 | 50.05 | |
| ATOM | 1027 | C | MET | | 282 | −31.086 | 98.112 | −70.770 | 1.00 | 49.94 | |
| ATOM | 1028 | O | MET | | 282 | −30.938 | 99.274 | −70.362 | 1.00 | 50.74 | |
| ATOM | 1029 | N | LEU | | 283 | −32.202 | 97.686 | −71.344 | 1.00 | 48.61 | |
| ATOM | 1030 | CA | LEU | | 283 | −33.344 | 98.564 | −71.500 | 1.00 | 48.61 | |
| ATOM | 1031 | CB | LEU | | 283 | −33.541 | 98.923 | −72.979 | 1.00 | 45.53 | |
| ATOM | 1032 | CG | LEU | | 283 | −32.426 | 99.762 | −73.629 | 1.00 | 46.07 | |
| ATOM | 1033 | CD1 | LEU | | 283 | −32.831 | 100.189 | −75.043 | 1.00 | 46.26 | |
| ATOM | 1034 | CD2 | LEU | | 283 | −32.158 | 101.005 | −72.796 | 1.00 | 46.21 | |
| ATOM | 1035 | C | LEU | | 283 | −34.554 | 97.828 | −70.964 | 1.00 | 50.44 | |
| ATOM | 1036 | O | LEU | | 283 | −34.693 | 96.632 | −71.194 | 1.00 | 49.45 | |
| ATOM | 1037 | N | ASP | | 284 | −35.417 | 98.511 | −70.216 | 1.00 | 53.04 | |
| ATOM | 1038 | CA | ASP | | 284 | −36.606 | 97.829 | −69.716 | 1.00 | 56.45 | |
| ATOM | 1039 | CB | ASP | | 284 | −37.127 | 98.448 | −68.398 | 1.00 | 56.47 | |
| ATOM | 1040 | CG | ASP | | 284 | −37.609 | 99.892 | −68.548 | 1.00 | 56.20 | |
| ATOM | 1041 | OD1 | ASP | | 284 | −38.024 | 100.291 | −69.657 | 1.00 | 55.37 | |
| ATOM | 1042 | OD2 | ASP | | 284 | −37.593 | 100.623 | −67.530 | 1.00 | 56.55 | |
| ATOM | 1043 | C | ASP | | 284 | −37.677 | 97.878 | −70.809 | 1.00 | 57.77 | |
| ATOM | 1044 | O | ASP | | 284 | −37.495 | 98.527 | −71.832 | 1.00 | 56.14 | |
| ATOM | 1045 | N | LYS | | 285 | −38.785 | 97.185 | −70.589 | 1.00 | 60.53 | |
| ATOM | 1046 | CA | LYS | | 285 | −39.870 | 97.136 | −71.562 | 1.00 | 62.34 | |
| ATOM | 1047 | CB | LYS | | 285 | −41.027 | 96.323 | −70.983 | 1.00 | 64.39 | |
| ATOM | 1048 | CG | LYS | | 285 | −41.474 | 96.804 | −69.621 | 1.00 | 67.74 | |
| ATOM | 1049 | CD | LYS | | 285 | −42.522 | 95.868 | −69.033 | 1.00 | 69.58 | |
| ATOM | 1050 | CE | LYS | | 285 | −42.837 | 96.237 | −67.587 | 1.00 | 71.14 | |
| ATOM | 1051 | NZ | LYS | | 285 | −41.661 | 96.089 | −66.665 | 1.00 | 71.01 | |
| ATOM | 1052 | C | LYS | | 285 | −40.399 | 98.477 | −72.085 | 1.00 | 61.37 | |
| ATOM | 1053 | O | LYS | | 285 | −41.191 | 98.489 | −73.018 | 1.00 | 62.10 | |
| ATOM | 1054 | N | ASP | | 286 | −39.972 | 99.594 | −71.504 | 1.00 | 59.71 | |
| ATOM | 1055 | CA | ASP | | 286 | −40.450 | 100.902 | −71.955 | 1.00 | 58.63 | |
| ATOM | 1056 | CB | ASP | | 286 | −41.104 | 101.685 | −70.796 | 1.00 | 55.55 | |
| ATOM | 1057 | CG | ASP | | 286 | −42.263 | 100.924 | −70.147 | 1.00 | 54.69 | |
| ATOM | 1058 | OD1 | ASP | | 286 | −43.056 | 100.323 | −70.906 | 1.00 | 53.87 | |
| ATOM | 1059 | OD2 | ASP | | 286 | −42.386 | 100.927 | −68.892 | 1.00 | 52.13 | |
| ATOM | 1060 | C | ASP | | 286 | −39.318 | 101.725 | −72.550 | 1.00 | 58.98 | |
| ATOM | 1061 | O | ASP | | 286 | −39.468 | 102.922 | −72.818 | 1.00 | 59.33 | |
| ATOM | 1062 | N | GLY | | 287 | −38.177 | 101.082 | −72.749 | 1.00 | 58.42 | |
| ATOM | 1063 | CA | GLY | | 287 | −37.045 | 101.776 | −73.321 | 1.00 | 55.33 | |
| ATOM | 1064 | C | GLY | | 287 | −36.218 | 102.535 | −72.313 | 1.00 | 55.12 | |
| ATOM | 1065 | O | GLY | | 287 | −35.337 | 103.302 | −72.706 | 1.00 | 55.89 | |
| ATOM | 1066 | N | HIS | | 288 | −36.503 | 102.364 | −71.021 | 1.00 | 53.99 | |
| ATOM | 1067 | CA | HIS | | 288 | −35.700 | 103.031 | −69.985 | 1.00 | 52.87 | |
| ATOM | 1068 | CB | HIS | | 288 | −36.535 | 103.298 | −68.735 | 1.00 | 54.12 | |
| ATOM | 1069 | CG | HIS | | 288 | −37.412 | 104.500 | −68.849 | 1.00 | 54.36 | |
| ATOM | 1070 | CD2 | HIS | | 288 | −38.754 | 104.620 | −68.949 | 1.00 | 54.12 | |
| ATOM | 1071 | ND1 | HIS | | 288 | −36.906 | 105.783 | −68.882 | 1.00 | 54.49 | |
| ATOM | 1072 | CE1 | HIS | | 288 | −37.904 | 106.642 | −68.993 | 1.00 | 55.05 | |
| ATOM | 1073 | NE2 | HIS | | 288 | −39.037 | 105.962 | −69.035 | 1.00 | 54.04 | |
| ATOM | 1074 | C | HIS | | 288 | −34.468 | 102.199 | −69.620 | 1.00 | 50.81 | |
| ATOM | 1075 | O | HIS | | 288 | −34.501 | 100.967 | −69.649 | 1.00 | 49.80 | |
| ATOM | 1076 | N | ILE | | 289 | −33.388 | 102.882 | −69.270 | 1.00 | 48.66 | |
| ATOM | 1077 | CA | ILE | | 289 | −32.133 | 102.231 | −68.928 | 1.00 | 45.41 | |
| ATOM | 1078 | CB | ILE | | 289 | −31.022 | 103.293 | −68.889 | 1.00 | 46.00 | |
| ATOM | 1079 | CG2 | ILE | | 289 | −30.430 | 103.389 | −67.495 | 1.00 | 46.37 | |
| ATOM | 1080 | CG1 | ILE | | 289 | −29.994 | 103.011 | −69.984 | 1.00 | 47.38 | |
| ATOM | 1081 | CD1 | ILE | | 289 | −28.768 | 103.925 | −69.940 | 1.00 | 48.92 | |
| ATOM | 1082 | C | ILE | | 289 | −32.132 | 101.434 | −67.604 | 1.00 | 45.12 | |
| ATOM | 1083 | O | ILE | | 289 | −32.836 | 101.778 | −66.648 | 1.00 | 44.61 | |
| ATOM | 1084 | N | LYS | | 290 | −31.341 | 100.359 | −67.564 | 1.00 | 42.74 | |
| ATOM | 1085 | CA | LYS | | 290 | −31.213 | 99.537 | −66.361 | 1.00 | 40.63 | |
| ATOM | 1086 | CB | LYS | | 290 | −32.108 | 98.286 | −66.403 | 1.00 | 38.79 | |
| ATOM | 1087 | CG | LYS | | 290 | −33.506 | 98.445 | −65.867 | 1.00 | 40.40 | |
| ATOM | 1088 | CD | LYS | | 290 | −33.569 | 98.783 | −64.376 | 1.00 | 40.88 | |
| ATOM | 1089 | CE | LYS | | 290 | −35.040 | 98.949 | −63.917 | 1.00 | 41.34 | |
| ATOM | 1090 | NZ | LYS | | 290 | −35.202 | 99.399 | −62.491 | 1.00 | 41.88 | |
| ATOM | 1091 | C | LYS | | 290 | −29.780 | 99.076 | −66.203 | 1.00 | 40.58 | |
| ATOM | 1092 | O | LYS | | 290 | −29.315 | 98.213 | −66.959 | 1.00 | 38.39 | |
| ATOM | 1093 | N | ILE | | 291 | −29.095 | 99.659 | −65.221 | 1.00 | 40.94 | |
| ATOM | 1094 | CA | ILE | | 291 | −27.726 | 99.314 | −64.899 | 1.00 | 42.72 | |
| ATOM | 1095 | CB | ILE | | 291 | −27.135 | 100.321 | −63.935 | 1.00 | 41.62 | |
| ATOM | 1096 | CG2 | ILE | | 291 | −25.725 | 99.908 | −63.581 | 1.00 | 43.00 | |
| ATOM | 1097 | CG1 | ILE | | 291 | −27.179 | 101.715 | −64.579 | 1.00 | 44.50 | |
| ATOM | 1098 | CD1 | ILE | | 291 | −26.529 | 102.835 | −63.793 | 1.00 | 39.86 | |
| ATOM | 1099 | C | ILE | | 291 | −27.760 | 97.937 | −64.242 | 1.00 | 42.95 | |
| ATOM | 1100 | O | ILE | | 291 | −28.153 | 97.791 | −63.099 | 1.00 | 42.12 | |
| ATOM | 1101 | N | THR | | 292 | −27.355 | 96.933 | −65.001 | 1.00 | 46.30 | |
| ATOM | 1102 | CA | THR | | 292 | −27.341 | 95.543 | −64.579 | 1.00 | 50.75 | |
| ATOM | 1103 | CB | THR | | 292 | −27.473 | 94.688 | −65.814 | 1.00 | 49.64 | |
| ATOM | 1104 | OG1 | THR | | 292 | −28.211 | 93.514 | −65.489 | 1.00 | 50.14 | |
| ATOM | 1105 | CG2 | THR | | 292 | −26.108 | 94.330 | −66.360 | 1.00 | 50.90 | |
| ATOM | 1106 | C | THR | | 292 | −26.045 | 95.219 | −63.844 | 1.00 | 53.08 | |
| ATOM | 1107 | O | THR | | 292 | −25.023 | 95.744 | −64.205 | 1.00 | 54.20 | |
| ATOM | 1108 | N | ASP | | 293 | −26.065 | 94.357 | −62.832 | 1.00 | 58.90 | |
| ATOM | 1109 | CA | ASP | | 293 | −24.821 | 94.066 | −62.078 | 1.00 | 65.40 | |
| ATOM | 1110 | CB | ASP | | 293 | −25.132 | 93.760 | −60.602 | 1.00 | 68.31 | |
| ATOM | 1111 | CG | ASP | | 293 | −23.910 | 93.957 | −59.688 | 1.00 | 71.75 | |
| ATOM | 1112 | OD1 | ASP | | 293 | −22.797 | 93.515 | −60.066 | 1.00 | 72.43 | |
| ATOM | 1113 | OD2 | ASP | | 293 | −24.066 | 94.558 | −58.588 | 1.00 | 72.94 | |
| ATOM | 1114 | C | ASP | | 293 | −23.898 | 92.954 | −62.585 | 1.00 | 66.34 | |
| ATOM | 1115 | O | ASP | | 293 | −23.428 | 92.142 | −61.795 | 1.00 | 67.07 | |
| ATOM | 1116 | N | PHE | | 294 | −23.619 | 92.922 | −63.881 | 1.00 | 68.29 | |
| ATOM | 1117 | CA | PHE | | 294 | −22.743 | 91.888 | −64.429 | 1.00 | 71.15 | |
| ATOM | 1118 | CB | PHE | | 294 | −23.363 | 90.512 | −64.170 | 1.00 | 70.29 | |
| ATOM | 1119 | CG | PHE | | 294 | −24.852 | 90.465 | −64.391 | 1.00 | 68.39 | |
| ATOM | 1120 | CD1 | PHE | | 294 | −25.374 | 90.204 | −65.653 | 1.00 | 67.64 | |
| ATOM | 1121 | CD2 | PHE | | 294 | −25.733 | 90.702 | −63.334 | 1.00 | 67.17 | |
| ATOM | 1122 | CE1 | PHE | | 294 | −26.752 | 90.171 | −65.864 | 1.00 | 67.40 | |
| ATOM | 1123 | CE2 | PHE | | 294 | −27.112 | 90.672 | −63.527 | 1.00 | 67.56 | |
| ATOM | 1124 | CZ | PHE | | 294 | −27.624 | 90.405 | −64.801 | 1.00 | 67.17 | |
| ATOM | 1125 | C | PHE | | 294 | −22.446 | 92.054 | −65.921 | 1.00 | 74.06 | |
| ATOM | 1126 | O | PHE | | 294 | −23.146 | 92.787 | −66.637 | 1.00 | 74.81 | |
| ATOM | 1127 | N | GLY | | 295 | −21.405 | 91.370 | −66.387 | 1.00 | 75.03 | |
| ATOM | 1128 | CA | GLY | | 295 | −21.057 | 91.448 | −67.794 | 1.00 | 77.37 | |
| ATOM | 1129 | C | GLY | | 295 | −21.847 | 90.436 | −68.605 | 1.00 | 78.99 | |
| ATOM | 1130 | O | GLY | | 295 | −21.644 | 89.232 | −68.449 | 1.00 | 77.61 | |
| ATOM | 1131 | N | ALA | | 296 | −22.744 | 90.923 | −69.468 | 1.00 | 81.84 | |
| ATOM | 1132 | CA | ALA | | 296 | −23.589 | 90.056 | −70.301 | 1.00 | 83.30 | |
| ATOM | 1133 | CB | ALA | | 296 | −24.207 | 90.857 | −71.446 | 1.00 | 82.28 | |
| ATOM | 1134 | C | ALA | | 296 | −22.814 | 88.866 | −70.859 | 1.00 | 84.74 | |
| ATOM | 1135 | O | ALA | | 296 | −22.495 | 87.925 | −70.129 | 1.00 | 85.55 | |
| ATOM | 1136 | N | THR | | 313 | −14.714 | 92.203 | −75.369 | 1.00 | 60.91 | |
| ATOM | 1137 | CA | THR | | 313 | −14.670 | 93.623 | −75.035 | 1.00 | 60.64 | |
| ATOM | 1138 | CB | THR | | 313 | −15.922 | 94.044 | −74.210 | 1.00 | 63.10 | |
| ATOM | 1139 | OG1 | THR | | 313 | −16.167 | 93.077 | −73.179 | 1.00 | 64.44 | |
| ATOM | 1140 | CG2 | THR | | 313 | −17.153 | 94.165 | −75.111 | 1.00 | 65.78 | |
| ATOM | 1141 | C | THR | | 313 | −13.420 | 94.061 | −74.252 | 1.00 | 58.49 | |
| ATOM | 1142 | O | THR | | 313 | −13.128 | 93.540 | −73.176 | 1.00 | 57.70 | |
| ATOM | 1143 | N | PRO | | 314 | −12.661 | 95.022 | −74.803 | 1.00 | 55.65 | |
| ATOM | 1144 | CD | PRO | | 314 | −12.751 | 95.465 | −76.206 | 1.00 | 54.61 | |
| ATOM | 1145 | CA | PRO | | 314 | −11.447 | 95.537 | −74.149 | 1.00 | 55.15 | |
| ATOM | 1146 | CB | PRO | | 314 | −10.831 | 96.445 | −75.212 | 1.00 | 53.14 | |
| ATOM | 1147 | CG | PRO | | 314 | −11.326 | 95.856 | −76.510 | 1.00 | 55.07 | |
| ATOM | 1148 | C | PRO | | 314 | −11.816 | 96.331 | −72.891 | 1.00 | 55.44 | |
| ATOM | 1149 | O | PRO | | 314 | −12.652 | 97.240 | −72.954 | 1.00 | 58.62 | |
| ATOM | 1150 | N | GLU | | 315 | −11.217 | 95.992 | −71.752 | 1.00 | 54.65 | |
| ATOM | 1151 | CA | GLU | | 315 | −11.486 | 96.709 | −70.498 | 1.00 | 52.63 | |
| ATOM | 1152 | CB | GLU | | 315 | −10.772 | 96.041 | −69.326 | 1.00 | 55.03 | |
| ATOM | 1153 | CG | GLU | | 315 | −11.462 | 94.835 | −68.718 | 1.00 | 59.66 | |
| ATOM | 1154 | CD | GLU | | 315 | −11.720 | 95.019 | −67.225 | 1.00 | 61.87 | |
| ATOM | 1155 | OE1 | GLU | | 315 | −12.813 | 95.521 | −66.858 | 1.00 | 64.01 | |
| ATOM | 1156 | OE2 | GLU | | 315 | −10.821 | 94.681 | −66.422 | 1.00 | 62.85 | |
| ATOM | 1157 | C | GLU | | 315 | −10.973 | 98.141 | −70.578 | 1.00 | 51.76 | |
| ATOM | 1158 | O | GLU | | 315 | −9.901 | 98.381 | −71.126 | 1.00 | 50.53 | |
| ATOM | 1159 | N | TYR | | 316 | −11.723 | 99.099 | −70.037 | 1.00 | 49.20 | |
| ATOM | 1160 | CA | TYR | | 316 | −11.262 | 100.487 | −70.037 | 1.00 | 45.83 | |
| ATOM | 1161 | CB | TYR | | 316 | −12.120 | 101.355 | −70.954 | 1.00 | 45.08 | |
| ATOM | 1162 | CG | TYR | | 316 | −11.603 | 101.488 | −72.365 | 1.00 | 43.63 | |
| ATOM | 1163 | CD1 | TYR | | 316 | −11.992 | 100.596 | −73.353 | 1.00 | 42.33 | |
| ATOM | 1164 | CE1 | TYR | | 316 | −11.590 | 100.765 | −74.671 | 1.00 | 42.22 | |
| ATOM | 1165 | CD2 | TYR | | 316 | −10.773 | 102.563 | −72.731 | 1.00 | 43.91 | |
| ATOM | 1166 | CE2 | TYR | | 316 | −10.363 | 102.743 | −74.056 | 1.00 | 40.77 | |
| ATOM | 1167 | CZ | TYR | | 316 | −10.782 | 101.837 | −75.014 | 1.00 | 41.68 | |
| ATOM | 1168 | OH | TYR | | 316 | −10.400 | 101.988 | −76.327 | 1.00 | 44.89 | |
| ATOM | 1169 | C | TYR | | 316 | −11.312 | 101.038 | −68.614 | 1.00 | 46.24 | |
| ATOM | 1170 | O | TYR | | 316 | −12.122 | 101.930 | −68.287 | 1.00 | 46.53 | |
| ATOM | 1171 | N | LEU | | 317 | −10.472 | 100.468 | −67.759 | 1.00 | 43.22 | |
| ATOM | 1172 | CA | LEU | | 317 | −10.397 | 100.902 | −66.377 | 1.00 | 41.82 | |
| ATOM | 1173 | CB | LEU | | 317 | −9.395 | 100.053 | −65.582 | 1.00 | 38.88 | |
| ATOM | 1174 | CG | LEU | | 317 | −9.778 | 98.578 | −65.467 | 1.00 | 39.79 | |
| ATOM | 1175 | CD1 | LEU | | 317 | −8.704 | 97.838 | −64.631 | 1.00 | 35.92 | |
| ATOM | 1176 | CD2 | LEU | | 317 | −11.196 | 98.444 | −64.855 | 1.00 | 33.82 | |
| ATOM | 1177 | C | LEU | | 317 | −9.955 | 102.349 | −66.354 | 1.00 | 41.08 | |
| ATOM | 1178 | O | LEU | | 317 | −9.159 | 102.777 | −67.188 | 1.00 | 40.65 | |
| ATOM | 1179 | N | ALA | | 318 | −10.470 | 103.098 | −65.393 | 1.00 | 40.28 | |
| ATOM | 1180 | CA | ALA | | 318 | −10.109 | 104.494 | −65.280 | 1.00 | 40.73 | |
| ATOM | 1181 | CB | ALA | | 318 | −10.853 | 105.129 | −64.092 | 1.00 | 38.50 | |
| ATOM | 1182 | C | ALA | | 318 | −8.604 | 104.570 | −65.061 | 1.00 | 39.60 | |
| ATOM | 1183 | O | ALA | | 318 | −8.029 | 103.712 | −64.432 | 1.00 | 40.78 | |
| ATOM | 1184 | N | PRO | | 319 | −7.950 | 105.604 | −65.582 | 1.00 | 39.37 | |
| ATOM | 1185 | CD | PRO | | 319 | −8.415 | 106.643 | −66.505 | 1.00 | 39.04 | |
| ATOM | 1186 | CA | PRO | | 319 | −6.511 | 105.711 | −65.377 | 1.00 | 39.03 | |
| ATOM | 1187 | CB | PRO | | 319 | −6.155 | 107.065 | −66.012 | 1.00 | 39.95 | |
| ATOM | 1188 | CG | PRO | | 319 | −7.466 | 107.750 | −66.171 | 1.00 | 42.09 | |
| ATOM | 1189 | C | PRO | | 319 | −6.068 | 105.611 | −63.928 | 1.00 | 39.88 | |
| ATOM | 1190 | O | PRO | | 319 | −5.090 | 104.906 | −63.616 | 1.00 | 41.07 | |
| ATOM | 1191 | N | GLU | | 320 | −6.766 | 106.288 | −63.020 | 1.00 | 41.42 | |
| ATOM | 1192 | CA | GLU | | 320 | −6.338 | 106.211 | −61.616 | 1.00 | 42.37 | |
| ATOM | 1193 | CB | GLU | | 320 | −7.040 | 107.272 | −60.729 | 1.00 | 43.77 | |
| ATOM | 1194 | CG | GLU | | 320 | −8.528 | 107.061 | −60.511 | 1.00 | 44.04 | |
| ATOM | 1195 | CD | GLU | | 320 | −9.384 | 107.677 | −61.606 | 1.00 | 46.60 | |
| ATOM | 1196 | OE1 | GLU | | 320 | −8.905 | 107.784 | −62.759 | 1.00 | 46.24 | |
| ATOM | 1197 | OE2 | GLU | | 320 | −10.547 | 108.029 | −61.314 | 1.00 | 45.58 | |
| ATOM | 1198 | C | GLU | | 320 | −6.555 | 104.785 | −61.068 | 1.00 | 43.26 | |
| ATOM | 1199 | O | GLU | | S320 | −6.004 | 104.416 | −60.026 | 1.00 | 43.06 | |
| ATOM | 1200 | N | VAL | | 321 | −7.368 | 103.983 | −61.752 | 1.00 | 43.24 | |
| ATOM | 1201 | CA | VAL | | 321 | −7.527 | 102.616 | −61.303 | 1.00 | 45.22 | |
| ATOM | 1202 | CB | VAL | | 321 | −8.793 | 101.942 | −61.886 | 1.00 | 44.66 | |
| ATOM | 1203 | CG1 | VAL | | 321 | −8.723 | 100.458 | −61.658 | 1.00 | 42.53 | |
| ATOM | 1204 | CG2 | VAL | | 321 | −10.056 | 102.509 | −61.224 | 1.00 | 43.26 | |
| ATOM | 1205 | C | VAL | | 321 | −6.272 | 101.877 | −61.807 | 1.00 | 47.72 | |
| ATOM | 1206 | O | VAL | | 321 | −5.638 | 101.142 | −61.052 | 1.00 | 47.80 | |
| ATOM | 1207 | N | LEU | | 322 | −5.896 | 102.093 | −63.070 | 1.00 | 47.95 | |
| ATOM | 1208 | CA | LEU | | 322 | −4.715 | 101.428 | −63.624 | 1.00 | 49.76 | |
| ATOM | 1209 | CB | LEU | | 322 | −4.532 | 101.776 | −65.098 | 1.00 | 47.04 | |
| ATOM | 1210 | CG | LEU | | 322 | −5.572 | 101.131 | −65.995 | 1.00 | 46.64 | |
| ATOM | 1211 | CD1 | LEU | | 322 | −5.338 | 101.564 | −67.424 | 1.00 | 45.36 | |
| ATOM | 1212 | CD2 | LEU | | 322 | −5.482 | 99.601 | −65.844 | 1.00 | 47.97 | |
| ATOM | 1213 | C | LEU | | 322 | −3.419 | 101.770 | −62.898 | 1.00 | 50.74 | |
| ATOM | 1214 | O | LEU | | 322 | −2.587 | 100.890 | −62.624 | 1.00 | 50.48 | |
| ATOM | 1215 | N | GLU | | 323 | −3.246 | 103.046 | −62.584 | 1.00 | 50.19 | |
| ATOM | 1216 | CA | GLU | | 323 | −2.024 | 103.473 | −61.931 | 1.00 | 50.50 | |
| ATOM | 1217 | CB | GLU | | 323 | −1.723 | 104.930 | −62.303 | 1.00 | 49.54 | |
| ATOM | 1218 | CG | GLU | | 323 | −0.531 | 105.544 | −61.605 | 1.00 | 51.05 | |
| ATOM | 1219 | CD | GLU | | 323 | −0.121 | 106.897 | −62.196 | 1.00 | 52.86 | |
| ATOM | 1220 | OE1 | GLU | | 323 | −0.996 | 107.667 | −62.659 | 1.00 | 51.36 | |
| ATOM | 1221 | OE2 | GLU | | 323 | 1.088 | 107.199 | −62.173 | 1.00 | 53.36 | |
| ATOM | 1222 | C | GLU | | 323 | −1.976 | 103.279 | −60.423 | 1.00 | 51.07 | |
| ATOM | 1223 | O | GLU | | 323 | −1.038 | 102.670 | −59.924 | 1.00 | 53.24 | |
| ATOM | 1224 | N | ASP | | 324 | −2.976 | 103.758 | −59.689 | 1.00 | 51.06 | |
| ATOM | 1225 | CA | ASP | | 324 | −2.935 | 103.625 | −58.242 | 1.00 | 51.85 | |
| ATOM | 1226 | CB | ASP | | 324 | −2.998 | 105.000 | −57.569 | 1.00 | 53.82 | |
| ATOM | 1227 | CG | ASP | | 324 | −1.834 | 105.890 | −57.953 | 1.00 | 56.66 | |
| ATOM | 1228 | OD1 | ASP | | 324 | −0.719 | 105.351 | −58.197 | 1.00 | 58.19 | |
| ATOM | 1229 | OD2 | ASP | | 324 | −2.030 | 107.132 | −57.999 | 1.00 | 56.61 | |
| ATOM | 1230 | C | ASP | | 324 | −3.988 | 102.746 | −57.624 | 1.00 | 51.70 | |
| ATOM | 1231 | O | ASP | | 324 | −4.235 | 102.840 | −56.425 | 1.00 | 52.09 | |
| ATOM | 1232 | N | ASN | | 325 | −4.599 | 101.886 | −58.424 | 1.00 | 51.98 | |
| ATOM | 1233 | CA | ASN | | 325 | −5.650 | 100.995 | −57.930 | 1.00 | 52.58 | |
| ATOM | 1234 | CB | ASN | | 325 | −5.045 | 99.844 | −57.145 | 1.00 | 51.16 | |
| ATOM | 1235 | CG | ASN | | 325 | −6.033 | 98.728 | −56.940 | 1.00 | 52.85 | |
| ATOM | 1236 | OD1 | ASN | | 325 | −6.692 | 98.301 | −57.891 | 1.00 | 51.88 | |
| ATOM | 1237 | ND2 | ASN | | 325 | −6.143 | 98.239 | −55.709 | 1.00 | 53.08 | |
| ATOM | 1238 | C | ASN | | 325 | −6.677 | 101.719 | −57.046 | 1.00 | 53.38 | |
| ATOM | 1239 | O | ASN | | 325 | −7.292 | 101.126 | −56.156 | 1.00 | 53.07 | |
| ATOM | 1240 | N | ASP | | 326 | −6.860 | 103.003 | −57.320 | 1.00 | 53.86 | |
| ATOM | 1241 | CA | ASP | | 326 | −7.778 | 103.855 | −56.578 | 1.00 | 54.16 | |
| ATOM | 1242 | CB | ASP | | 326 | −7.126 | 105.233 | −56.459 | 1.00 | 57.67 | |
| ATOM | 1243 | CG | ASP | | 326 | −8.052 | 106.279 | −55.921 | 1.00 | 59.69 | |
| ATOM | 1244 | OD1 | ASP | | 326 | −7.591 | 107.424 | −55.741 | 1.00 | 62.38 | |
| ATOM | 1245 | OD2 | ASP | | 326 | −9.231 | 105.974 | −55.686 | 1.00 | 63.13 | |
| ATOM | 1246 | C | ASP | | 326 | −9.175 | 103.941 | −57.228 | 1.00 | 52.75 | |
| ATOM | 1247 | O | ASP | | 326 | −9.360 | 104.610 | −58.246 | 1.00 | 53.16 | |
| ATOM | 1248 | N | TYR | | 327 | −10.145 | 103.255 | −56.621 | 1.00 | 49.27 | |
| ATOM | 1249 | CA | TYR | | 327 | −11.521 | 103.229 | −57.104 | 1.00 | 47.81 | |
| ATOM | 1250 | CB | TYR | | 327 | −12.174 | 101.844 | −56.887 | 1.00 | 46.28 | |
| ATOM | 1251 | CG | TYR | | 327 | −11.801 | 100.745 | −57.861 | 1.00 | 45.89 | |
| ATOM | 1252 | CD1 | TYR | | 327 | −10.607 | 100.030 | −57.726 | 1.00 | 46.18 | |
| ATOM | 1253 | CE1 | TYR | | 327 | −10.265 | 99.026 | −58.620 | 1.00 | 45.79 | |
| ATOM | 1254 | CD2 | TYR | | 327 | −12.642 | 100.421 | −58.924 | 1.00 | 45.64 | |
| ATOM | 1255 | CE2 | TYR | | 327 | −12.305 | 99.420 | −59.828 | 1.00 | 46.87 | |
| ATOM | 1256 | CZ | TYR | | 327 | −11.113 | 98.722 | −59.672 | 1.00 | 48.27 | |
| ATOM | 1257 | OH | TYR | | 327 | −10.775 | 97.734 | −60.583 | 1.00 | 49.49 | |
| ATOM | 1258 | C | TYR | | 327 | −12.406 | 104.263 | −56.388 | 1.00 | 48.05 | |
| ATOM | 1259 | O | TYR | | 327 | −13.294 | 103.873 | −55.617 | 1.00 | 47.57 | |
| ATOM | 1260 | N | GLY | | 328 | −12.182 | 105.555 | −56.642 | 1.00 | 47.18 | |
| ATOM | 1261 | CA | GLY | | 328 | −13.003 | 106.592 | −56.028 | 1.00 | 46.32 | |
| ATOM | 1262 | C | GLY | | 328 | −14.168 | 107.043 | −56.905 | 1.00 | 46.65 | |
| ATOM | 1263 | O | GLY | | 328 | −14.212 | 106.706 | −58.091 | 1.00 | 47.60 | |
| ATOM | 1264 | N | ARG | | 329 | −15.100 | 107.806 | −56.332 | 1.00 | 45.55 | |
| ATOM | 1265 | CA | ARG | | 329 | −16.280 | 108.314 | −57.057 | 1.00 | 46.76 | |
| ATOM | 1266 | CB | ARG | | 329 | −16.745 | 109.667 | −56.494 | 1.00 | 48.48 | |
| ATOM | 1267 | CG | ARG | | 329 | −16.740 | 109.839 | −54.996 | 1.00 | 52.56 | |
| ATOM | 1268 | CD | ARG | | 329 | −16.236 | 111.251 | −54.601 | 1.00 | 54.15 | |
| ATOM | 1269 | NE | ARG | | 329 | −16.644 | 112.263 | −55.563 | 1.00 | 55.37 | |
| ATOM | 1270 | CZ | ARG | | 329 | −16.066 | 113.450 | −55.706 | 1.00 | 55.95 | |
| ATOM | 1271 | NH1 | ARG | | 329 | −15.038 | 113.807 | −54.948 | 1.00 | 55.06 | |
| ATOM | 1272 | NH2 | ARG | | 329 | −16.520 | 114.280 | −56.630 | 1.00 | 59.49 | |
| ATOM | 1273 | C | ARG | | 329 | −16.017 | 108.549 | −58.548 | 1.00 | 46.10 | |
| ATOM | 1274 | O | ARG | | 329 | −16.807 | 108.160 | −59.418 | 1.00 | 46.43 | |
| ATOM | 1275 | N | ALA | | 330 | −14.904 | 109.231 | −58.806 | 1.00 | 46.16 | |
| ATOM | 1276 | CA | ALA | | 330 | −14.463 | 109.614 | −60.134 | 1.00 | 45.40 | |
| ATOM | 1277 | CB | ALA | | 330 | −13.052 | 110.143 | −60.053 | 1.00 | 44.79 | |
| ATOM | 1278 | C | ALA | | 330 | −14.551 | 108.554 | −61.218 | 1.00 | 44.25 | |
| ATOM | 1279 | O | ALA | | 330 | −14.952 | 108.843 | −62.339 | 1.00 | 45.35 | |
| ATOM | 1280 | N | VAL | | 331 | −14.193 | 107.323 | −60.903 | 1.00 | 43.40 | |
| ATOM | 1281 | CA | VAL | | 331 | −14.235 | 106.295 | −61.938 | 1.00 | 42.86 | |
| ATOM | 1282 | CB | VAL | | 331 | −13.607 | 104.964 | −61.440 | 1.00 | 42.39 | |
| ATOM | 1283 | CG1 | VAL | | 331 | −12.330 | 105.257 | −60.703 | 1.00 | 40.94 | |
| ATOM | 1284 | CG2 | VAL | | 331 | −14.576 | 104.212 | −60.563 | 1.00 | 41.02 | |
| ATOM | 1285 | C | VAL | | 331 | −15.621 | 106.018 | −62.527 | 1.00 | 42.04 | |
| ATOM | 1286 | O | VAL | | 331 | −15.728 | 105.397 | −63.596 | 1.00 | 42.24 | |
| ATOM | 1287 | N | ASP | | 332 | −16.687 | 106.458 | −61.865 | 1.00 | 38.49 | |
| ATOM | 1288 | CA | ASP | | 332 | −17.983 | 106.192 | −62.457 | 1.00 | 39.57 | |
| ATOM | 1289 | CB | ASP | | 332 | −19.117 | 106.275 | −61.430 | 1.00 | 41.24 | |
| ATOM | 1290 | CG | ASP | | 332 | −19.090 | 105.143 | −60.432 | 1.00 | 43.04 | |
| ATOM | 1291 | OD1 | ASP | | 332 | −18.807 | 104.002 | −60.846 | 1.00 | 44.33 | |
| ATOM | 1292 | OD2 | ASP | | 332 | −19.371 | 105.388 | −59.236 | 1.00 | 42.62 | |
| ATOM | 1293 | C | ASP | | 332 | −18.231 | 107.187 | −63.588 | 1.00 | 37.82 | |
| ATOM | 1294 | O | ASP | | 332 | −18.927 | 106.888 | −64.568 | 1.00 | 37.16 | |
| ATOM | 1295 | N | TRP | | 333 | −17.652 | 108.371 | −63.446 | 1.00 | 37.40 | |
| ATOM | 1296 | CA | TRP | | 333 | −17.817 | 109.414 | −64.434 | 1.00 | 38.54 | |
| ATOM | 1297 | CB | TRP | | 333 | −17.321 | 110.745 | −63.853 | 1.00 | 40.23 | |
| ATOM | 1298 | CG | TRP | | 333 | −18.209 | 111.198 | −62.715 | 1.00 | 41.98 | |
| ATOM | 1299 | CD2 | TRP | | 333 | −19.644 | 111.173 | −62.688 | 1.00 | 39.22 | |
| ATOM | 1300 | CE2 | TRP | | 333 | −20.050 | 111.582 | −61.402 | 1.00 | 40.30 | |
| ATOM | 1301 | CE3 | TRP | | 333 | −20.629 | 110.829 | −63.625 | 1.00 | 38.40 | |
| ATOM | 1302 | CD1 | TRP | | 333 | −17.811 | 111.630 | −61.473 | 1.00 | 43.77 | |
| ATOM | 1303 | NE1 | TRP | | 333 | −18.912 | 111.859 | −60.680 | 1.00 | 42.72 | |
| ATOM | 1304 | CZ2 | TRP | | 333 | −21.397 | 111.670 | −61.026 | 1.00 | 39.06 | |
| ATOM | 1305 | CZ3 | TRP | | 333 | −21.967 | 110.915 | −63.254 | 1.00 | 38.33 | |
| ATOM | 1306 | CH2 | TRP | | 333 | −22.337 | 111.330 | −61.960 | 1.00 | 36.53 | |
| ATOM | 1307 | C | TRP | | 333 | −17.060 | 108.981 | −65.681 | 1.00 | 38.67 | |
| ATOM | 1308 | O | TRP | | 333 | −17.605 | 109.055 | −66.801 | 1.00 | 36.29 | |
| ATOM | 1309 | N | TRP | | 334 | −15.824 | 108.508 | −65.470 | 1.00 | 38.23 | |
| ATOM | 1310 | CA | TRP | | 334 | −14.979 | 107.985 | −66.546 | 1.00 | 35.70 | |
| ATOM | 1311 | CB | TRP | | 334 | −13.684 | 107.430 | −65.945 | 1.00 | 40.15 | |
| ATOM | 1312 | CG | TRP | | 334 | −12.897 | 106.601 | −66.905 | 1.00 | 45.37 | |
| ATOM | 1313 | CD2 | TRP | | 334 | −11.885 | 107.059 | −67.802 | 1.00 | 46.40 | |
| ATOM | 1314 | CE2 | TRP | | 334 | −11.478 | 105.951 | −68.572 | 1.00 | 47.80 | |
| ATOM | 1315 | CE3 | TRP | | 334 | −11.287 | 108.303 | −68.039 | 1.00 | 47.17 | |
| ATOM | 1316 | CD1 | TRP | | 334 | −13.054 | 105.266 | −67.154 | 1.00 | 46.66 | |
| ATOM | 1317 | NE1 | TRP | | 334 | −12.203 | 104.871 | −68.154 | 1.00 | 49.45 | |
| ATOM | 1318 | CZ2 | TRP | | 334 | −10.496 | 106.045 | −69.556 | 1.00 | 48.28 | |
| ATOM | 1319 | CZ3 | TRP | | 334 | −10.312 | 108.398 | −69.017 | 1.00 | 47.44 | |
| ATOM | 1320 | CH2 | TRP | | 334 | −9.927 | 107.276 | −69.767 | 1.00 | 47.35 | |
| ATOM | 1321 | C | TRP | | 334 | −15.814 | 106.882 | −67.206 | 1.00 | 32.98 | |
| ATOM | 1322 | O | TRP | | 334 | −15.976 | 106.805 | −68.446 | 1.00 | 29.28 | |
| ATOM | 1323 | N | GLY | | 335 | −16.388 | 106.040 | −66.359 | 1.00 | 32.62 | |
| ATOM | 1324 | CA | GLY | | 335 | −17.246 | 104.975 | −66.847 | 1.00 | 31.24 | |
| ATOM | 1325 | C | GLY | | 335 | −18.265 | 105.585 | −67.767 | 1.00 | 32.09 | |
| ATOM | 1326 | O | GLY | | 335 | −18.422 | 105.124 | −68.883 | 1.00 | 35.03 | |
| ATOM | 1327 | N | LEU | | 336 | −18.950 | 106.630 | −67.302 | 1.00 | 32.31 | |
| ATOM | 1328 | CA | LEU | | 336 | −19.966 | 107.316 | −68.103 | 1.00 | 31.74 | |
| ATOM | 1329 | CB | LEU | | 336 | −20.548 | 108.517 | −67.346 | 1.00 | 34.73 | |
| ATOM | 1330 | CG | LEU | | 336 | −21.643 | 109.289 | −68.099 | 1.00 | 35.62 | |
| ATOM | 1331 | CD1 | LEU | | 336 | −22.851 | 108.369 | −68.323 | 1.00 | 39.53 | |
| ATOM | 1332 | CD2 | LEU | | 336 | −22.039 | 110.545 | −67.321 | 1.00 | 38.52 | |
| ATOM | 1333 | C | LEU | | 336 | −19.299 | 107.801 | −69.376 | 1.00 | 32.10 | |
| ATOM | 1334 | O | LEU | | 336 | −19.747 | 107.488 | −70.486 | 1.00 | 30.32 | |
| ATOM | 1335 | N | GLY | | 337 | −18.231 | 108.578 | −69.198 | 1.00 | 30.47 | |
| ATOM | 1336 | CA | GLY | | 337 | −17.477 | 109.061 | −70.329 | 1.00 | 33.11 | |
| ATOM | 1337 | C | GLY | | 337 | −17.185 | 107.988 | −71.375 | 1.00 | 36.30 | |
| ATOM | 1338 | O | GLY | | 337 | −17.617 | 108.136 | −72.527 | 1.00 | 39.45 | |
| ATOM | 1339 | N | VAL | | 338 | −16.479 | 106.914 | −71.011 | 1.00 | 35.68 | |
| ATOM | 1340 | CA | VAL | | 338 | −16.186 | 105.844 | −71.987 | 1.00 | 34.88 | |
| ATOM | 1341 | CB | VAL | | 338 | −15.401 | 104.640 | −71.340 | 1.00 | 31.93 | |
| ATOM | 1342 | CG1 | VAL | | 338 | −15.010 | 103.655 | −72.393 | 1.00 | 29.67 | |
| ATOM | 1343 | CG2 | VAL | | 338 | −14.177 | 105.137 | −70.605 | 1.00 | 26.65 | |
| ATOM | 1344 | C | VAL | | 338 | −17.481 | 105.308 | −72.639 | 1.00 | 36.09 | |
| ATOM | 1345 | O | VAL | | 338 | −17.538 | 105.071 | −73.863 | 1.00 | 32.07 | |
| ATOM | 1346 | N | VAL | | 339 | −18.525 | 105.132 | −71.830 | 1.00 | 37.68 | |
| ATOM | 1347 | CA | VAL | | 339 | −19.805 | 104.657 | −72.378 | 1.00 | 39.88 | |
| ATOM | 1348 | CB | VAL | | 339 | −20.863 | 104.396 | −71.283 | 1.00 | 39.09 | |
| ATOM | 1349 | CG1 | VAL | | 339 | −22.073 | 103.703 | −71.881 | 1.00 | 39.83 | |
| ATOM | 1350 | CG2 | VAL | | 339 | −20.275 | 103.573 | −70.166 | 1.00 | 41.28 | |
| ATOM | 1351 | C | VAL | | 339 | −20.395 | 105.698 | −73.324 | 1.00 | 40.15 | |
| ATOM | 1352 | O | VAL | | 339 | −21.040 | 105.359 | −74.302 | 1.00 | 42.13 | |
| ATOM | 1353 | N | MET | | 340 | −20.184 | 106.973 | −73.042 | 1.00 | 42.58 | |
| ATOM | 1354 | CA | MET | | 340 | −20.744 | 107.986 | −73.923 | 1.00 | 46.52 | |
| ATOM | 1355 | CB | MET | | 340 | −21.231 | 109.194 | −73.131 | 1.00 | 45.49 | |
| ATOM | 1356 | CG | MET | | 340 | −22.389 | 108.841 | −72.275 | 1.00 | 49.72 | |
| ATOM | 1357 | SD | MET | | 340 | −23.278 | 110.256 | −71.668 | 1.00 | 53.94 | |
| ATOM | 1358 | CE | MET | | 340 | −21.879 | 111.190 | −70.904 | 1.00 | 54.43 | |
| ATOM | 1359 | C | MET | | 340 | −19.815 | 108.409 | −75.045 | 1.00 | 46.60 | |
| ATOM | 1360 | O | MET | | 340 | −20.173 | 109.222 | −75.910 | 1.00 | 47.26 | |
| ATOM | 1361 | N | TYR | | 341 | −18.606 | 107.887 | −75.035 | 1.00 | 45.25 | |
| ATOM | 1362 | CA | TYR | | 341 | −17.761 | 108.207 | −76.150 | 1.00 | 45.45 | |
| ATOM | 1363 | CB | TYR | | 341 | −16.292 | 108.076 | −75.787 | 1.00 | 44.57 | |
| ATOM | 1364 | CG | TYR | | 341 | −15.357 | 108.298 | −76.945 | 1.00 | 44.38 | |
| ATOM | 1365 | CD1 | TYR | | 341 | −15.388 | 107.468 | −78.071 | 1.00 | 44.83 | |
| ATOM | 1366 | CE1 | TYR | | 341 | −14.490 | 107.628 | −79.118 | 1.00 | 43.03 | |
| ATOM | 1367 | CD2 | TYR | | 341 | −14.403 | 109.298 | −76.895 | 1.00 | 46.38 | |
| ATOM | 1368 | CE2 | TYR | | 341 | −13.486 | 109.475 | −77.939 | 1.00 | 44.56 | |
| ATOM | 1369 | CZ | TYR | | 341 | −13.538 | 108.641 | −79.041 | 1.00 | 44.25 | |
| ATOM | 1370 | OH | TYR | | 341 | −12.652 | 108.844 | −80.063 | 1.00 | 41.93 | |
| ATOM | 1371 | C | TYR | | 341 | −18.177 | 107.170 | −77.188 | 1.00 | 45.21 | |
| ATOM | 1372 | O | TYR | | 341 | −18.176 | 107.451 | −78.382 | 1.00 | 44.90 | |
| ATOM | 1373 | N | GLU | | 342 | −18.608 | 105.993 | −76.729 | 1.00 | 45.89 | |
| ATOM | 1374 | CA | GLU | | 342 | −18.999 | 104.938 | −77.664 | 1.00 | 46.56 | |
| ATOM | 1375 | CB | GLU | | 342 | −19.023 | 103.555 | −77.006 | 1.00 | 47.47 | |
| ATOM | 1376 | CG | GLU | | 342 | −18.486 | 102.458 | −77.930 | 1.00 | 51.19 | |
| ATOM | 1377 | CD | GLU | | 342 | −18.742 | 101.013 | −77.446 | 1.00 | 53.52 | |
| ATOM | 1378 | OE1 | GLU | | 342 | −18.360 | 100.648 | −76.303 | 1.00 | 55.05 | |
| ATOM | 1379 | OE2 | GLU | | 342 | −19.316 | 100.232 | −78.233 | 1.00 | 51.05 | |
| ATOM | 1380 | C | GLU | | 342 | −20.311 | 105.165 | −78.353 | 1.00 | 45.51 | |
| ATOM | 1381 | O | GLU | | 342 | −20.346 | 105.169 | −79.566 | 1.00 | 47.31 | |
| ATOM | 1382 | N | MET | | 343 | −21.387 | 105.360 | −77.596 | 1.00 | 46.13 | |
| ATOM | 1383 | CA | MET | | 343 | −22.736 | 105.558 | −78.163 | 1.00 | 45.97 | |
| ATOM | 1384 | CB | MET | | 343 | −23.738 | 105.975 | −77.076 | 1.00 | 45.96 | |
| ATOM | 1385 | CG | MET | | 343 | −24.381 | 104.877 | −76.274 | 1.00 | 45.04 | |
| ATOM | 1386 | SD | MET | | 343 | −25.048 | 105.637 | −74.779 | 1.00 | 44.87 | |
| ATOM | 1387 | CE | MET | | 343 | −26.173 | 104.315 | −74.111 | 1.00 | 44.54 | |
| ATOM | 1388 | C | MET | | 343 | −22.830 | 106.589 | −79.277 | 1.00 | 47.60 | |
| ATOM | 1389 | O | MET | | 343 | −23.570 | 106.379 | −80.243 | 1.00 | 45.42 | |
| ATOM | 1390 | N | MET | | 344 | −22.113 | 107.707 | −79.114 | 1.00 | 48.72 | |
| ATOM | 1391 | CA | MET | | 344 | −22.120 | 108.800 | −80.083 | 1.00 | 51.33 | |
| ATOM | 1392 | CB | MET | | 344 | −21.906 | 110.152 | −79.363 | 1.00 | 50.03 | |
| ATOM | 1393 | CG | MET | | 344 | −23.080 | 110.600 | −78.422 | 1.00 | 51.59 | |
| ATOM | 1394 | SD | MET | | 344 | −22.914 | 112.225 | −77.512 | 1.00 | 51.38 | |
| ATOM | 1395 | CE | MET | | 344 | −21.418 | 111.976 | −76.565 | 1.00 | 50.37 | |
| ATOM | 1396 | C | MET | | 344 | −21.060 | 108.598 | −81.186 | 1.00 | 54.81 | |
| ATOM | 1397 | O | MET | | 344 | −21.308 | 108.906 | −82.357 | 1.00 | 55.93 | |
| ATOM | 1398 | N | CYS | | 345 | −19.881 | 108.085 | −80.806 | 1.00 | 56.27 | |
| ATOM | 1399 | CA | CYS | | 345 | −18.792 | 107.826 | −81.745 | 1.00 | 53.60 | |
| ATOM | 1400 | CB | CYS | | 345 | −17.446 | 107.898 | −81.033 | 1.00 | 53.35 | |
| ATOM | 1401 | SG | CYS | | 345 | −16.911 | 109.518 | −80.366 | 1.00 | 51.93 | |
| ATOM | 1402 | C | CYS | | 345 | −18.930 | 106.460 | −82.434 | 1.00 | 54.34 | |
| ATOM | 1403 | O | CYS | | 345 | −18.421 | 106.275 | −83.532 | 1.00 | 54.10 | |
| ATOM | 1404 | N | GLY | | 346 | −19.630 | 105.519 | −81.797 | 1.00 | 55.46 | |
| ATOM | 1405 | CA | GLY | | 346 | −19.823 | 104.182 | −82.359 | 1.00 | 56.53 | |
| ATOM | 1406 | C | GLY | | 346 | −18.639 | 103.222 | −82.176 | 1.00 | 58.54 | |
| ATOM | 1407 | O | GLY | | 346 | −18.619 | 102.110 | −82.711 | 1.00 | 58.51 | |
| ATOM | 1408 | N | ARG | | 347 | −17.648 | 103.643 | −81.400 | 1.00 | 59.41 | |
| ATOM | 1409 | CA | ARG | | 347 | −16.457 | 102.832 | −81.187 | 1.00 | 60.48 | |
| ATOM | 1410 | CB | ARG | | 347 | −15.565 | 102.910 | −82.426 | 1.00 | 62.58 | |
| ATOM | 1411 | CG | ARG | | 347 | −15.306 | 104.335 | −82.851 | 1.00 | 65.67 | |
| ATOM | 1412 | CD | ARG | | 347 | −14.072 | 104.457 | −83.713 | 1.00 | 70.23 | |
| ATOM | 1413 | NE | ARG | | 347 | −13.477 | 105.793 | −83.627 | 1.00 | 74.54 | |
| ATOM | 1414 | CZ | ARG | | 347 | −14.030 | 106.899 | −84.124 | 1.00 | 77.20 | |
| ATOM | 1415 | NH1 | ARG | | 347 | −15.201 | 106.834 | −84.751 | 1.00 | 75.57 | |
| ATOM | 1416 | NH2 | ARG | | 347 | −13.408 | 108.075 | −84.001 | 1.00 | 78.60 | |
| ATOM | 1417 | C | ARG | | 347 | −15.717 | 103.403 | −79.982 | 1.00 | 59.56 | |
| ATOM | 1418 | O | ARG | | 347 | −15.957 | 104.554 | −79.596 | 1.00 | 58.79 | |
| ATOM | 1419 | N | LEU | | 348 | −14.813 | 102.616 | −79.402 | 1.00 | 57.45 | |
| ATOM | 1420 | CA | LEU | | 348 | −14.080 | 103.053 | −78.224 | 1.00 | 55.76 | |
| ATOM | 1421 | CB | LEU | | 348 | −13.539 | 101.834 | −77.486 | 1.00 | 53.62 | |
| ATOM | 1422 | CG | LEU | | 348 | −14.603 | 100.859 | −76.953 | 1.00 | 52.62 | |
| ATOM | 1423 | CD1 | LEU | | 348 | −13.980 | 99.518 | −76.642 | 1.00 | 54.33 | |
| ATOM | 1424 | CD2 | LEU | | 348 | −15.253 | 101.425 | −75.707 | 1.00 | 54.19 | |
| ATOM | 1425 | C | LEU | | 348 | −12.971 | 104.037 | −78.586 | 1.00 | 58.09 | |
| ATOM | 1426 | O | LEU | | 348 | −12.520 | 104.098 | −79.719 | 1.00 | 59.07 | |
| ATOM | 1427 | N | PRO | | 349 | −12.517 | 104.835 | −77.625 | 1.00 | 59.01 | |
| ATOM | 1428 | CD | PRO | | 349 | −12.930 | 105.013 | −76.230 | 1.00 | 59.70 | |
| ATOM | 1429 | CA | PRO | | 349 | −11.464 | 105.777 | −77.993 | 1.00 | 61.12 | |
| ATOM | 1430 | CB | PRO | | 349 | −11.156 | 106.502 | −76.671 | 1.00 | 60.60 | |
| ATOM | 1431 | CG | PRO | | 349 | −11.694 | 105.606 | −75.620 | 1.00 | 60.92 | |
| ATOM | 1432 | C | PRO | | 349 | −10.225 | 105.212 | −78.671 | 1.00 | 62.77 | |
| ATOM | 1433 | O | PRO | | 349 | −9.471 | 105.945 | −79.300 | 1.00 | 63.54 | |
| ATOM | 1434 | N | PHE | | 350 | −10.008 | 103.914 | −78.576 | 1.00 | 64.60 | |
| ATOM | 1435 | CA | PHE | | 350 | −8.819 | 103.351 | −79.205 | 1.00 | 66.55 | |
| ATOM | 1436 | CB | PHE | | 350 | −7.768 | 103.028 | −78.130 | 1.00 | 63.72 | |
| ATOM | 1437 | CG | PHE | | 350 | −7.373 | 104.204 | −77.264 | 1.00 | 61.36 | |
| ATOM | 1438 | CD1 | PHE | | 350 | −7.537 | 104.151 | −75.880 | 1.00 | 61.07 | |
| ATOM | 1439 | CD2 | PHE | | 350 | −6.765 | 105.331 | −77.815 | 1.00 | 61.06 | |
| ATOM | 1440 | CE1 | PHE | | 350 | −7.097 | 105.200 | −75.057 | 1.00 | 59.73 | |
| ATOM | 1441 | CE2 | PHE | | 350 | −6.319 | 106.385 | −76.998 | 1.00 | 58.34 | |
| ATOM | 1442 | CZ | PHE | | 350 | −6.483 | 106.316 | −75.620 | 1.00 | 58.70 | |
| ATOM | 1443 | C | PHE | | 350 | −9.173 | 102.076 | −79.974 | 1.00 | 69.12 | |
| ATOM | 1444 | O | PHE | | 350 | −8.336 | 101.197 | −80.134 | 1.00 | 69.42 | |
| ATOM | 1445 | N | TYR | | 351 | −10.414 | 101.990 | −80.447 | 1.00 | 72.87 | |
| ATOM | 1446 | CA | TYR | | 351 | −10.928 | 100.814 | −81.162 | 1.00 | 76.20 | |
| ATOM | 1447 | CB | TYR | | 351 | −12.249 | 101.146 | −81.860 | 1.00 | 77.72 | |
| ATOM | 1448 | CG | TYR | | 351 | −12.068 | 101.741 | −83.240 | 1.00 | 80.61 | |
| ATOM | 1449 | CD1 | TYR | | 351 | −12.807 | 101.265 | −84.323 | 1.00 | 80.64 | |
| ATOM | 1450 | CE1 | TYR | | 351 | −12.640 | 101.804 | −85.595 | 1.00 | 81.66 | |
| ATOM | 1451 | CD2 | TYR | | 351 | −11.154 | 102.777 | −83.467 | 1.00 | 80.71 | |
| ATOM | 1452 | CE2 | TYR | | 351 | −10.982 | 103.321 | −84.734 | 1.00 | 81.04 | |
| ATOM | 1453 | CZ | TYR | | 351 | −11.727 | 102.831 | −85.787 | 1.00 | 81.43 | |
| ATOM | 1454 | OH | TYR | | 351 | −11.578 | 103.376 | −87.033 | 1.00 | 82.80 | |
| ATOM | 1455 | C | TYR | | 351 | −9.991 | 100.174 | −82.187 | 1.00 | 77.87 | |
| ATOM | 1456 | O | TYR | | 351 | −10.102 | 98.975 | −82.470 | 1.00 | 78.58 | |
| ATOM | 1457 | N | ASN | | 352 | −9.093 | 100.960 | −82.768 | 1.00 | 78.54 | |
| ATOM | 1458 | CA | ASN | | 352 | −8.168 | 100.394 | −83.734 | 1.00 | 80.51 | |
| ATOM | 1459 | CB | ASN | | 352 | −7.914 | 101.381 | −84.874 | 1.00 | 81.11 | |
| ATOM | 1460 | CG | ASN | | 352 | −7.613 | 102.771 | −84.382 | 1.00 | 81.90 | |
| ATOM | 1461 | OD1 | ASN | | 352 | −7.431 | 103.688 | −85.173 | 1.00 | 81.64 | |
| ATOM | 1462 | ND2 | ASN | | 352 | −7.561 | 102.939 | −83.067 | 1.00 | 82.39 | |
| ATOM | 1463 | C | ASN | | 352 | −6.853 | 99.971 | −83.070 | 1.00 | 81.13 | |
| ATOM | 1464 | O | ASN | | 352 | −6.638 | 98.783 | −82.830 | 1.00 | 81.84 | |
| ATOM | 1465 | N | GLN | | 353 | −5.991 | 100.932 | −82.753 | 1.00 | 81.20 | |
| ATOM | 1466 | CA | GLN | | 353 | −4.704 | 100.640 | −82.124 | 1.00 | 81.66 | |
| ATOM | 1467 | CB | GLN | | 353 | −4.330 | 101.762 | −81.165 | 1.00 | 81.11 | |
| ATOM | 1468 | CG | GLN | | 353 | −4.343 | 103.105 | −81.834 | 1.00 | 81.13 | |
| ATOM | 1469 | CD | GLN | | 353 | −3.603 | 104.136 | −81.040 | 1.00 | 83.01 | |
| ATOM | 1470 | OE1 | GLN | | 353 | −2.406 | 103.992 | −80.786 | 1.00 | 82.60 | |
| ATOM | 1471 | NE2 | GLN | | 353 | −4.305 | 105.191 | −80.638 | 1.00 | 83.18 | |
| ATOM | 1472 | C | GLN | | 353 | −4.640 | 99.298 | −81.401 | 1.00 | 82.23 | |
| ATOM | 1473 | O | GLN | | 353 | −5.395 | 99.046 | −80.468 | 1.00 | 82.60 | |
| ATOM | 1474 | N | ASP | | 354 | −3.724 | 98.450 | −81.866 | 1.00 | 83.56 | |
| ATOM | 1475 | CA | ASP | | 354 | −3.486 | 97.104 | −81.342 | 1.00 | 84.13 | |
| ATOM | 1476 | CB | ASP | | 354 | −2.036 | 96.999 | −80.852 | 1.00 | 85.68 | |
| ATOM | 1477 | CG | ASP | | 354 | −1.518 | 95.567 | −80.842 | 1.00 | 87.94 | |
| ATOM | 1478 | OD1 | ASP | | 354 | −2.119 | 94.708 | −80.151 | 1.00 | 90.28 | |
| ATOM | 1479 | OD2 | ASP | | 354 | −0.504 | 95.302 | −81.528 | 1.00 | 87.84 | |
| ATOM | 1480 | C | ASP | | 354 | −4.459 | 96.681 | −80.233 | 1.00 | 83.94 | |
| ATOM | 1481 | O | ASP | | 354 | −5.641 | 96.437 | −80.498 | 1.00 | 84.30 | |
| ATOM | 1482 | N | HIS | | 355 | −3.964 | 96.586 | −79.000 | 1.00 | 82.80 | |
| ATOM | 1483 | CA | HIS | | 355 | −4.799 | 96.195 | −77.879 | 1.00 | 81.64 | |
| ATOM | 1484 | CB | HIS | | 355 | −5.490 | 94.878 | −78.157 | 1.00 | 83.58 | |
| ATOM | 1485 | CG | HIS | | 355 | −6.368 | 94.442 | −77.037 | 1.00 | 86.16 | |
| ATOM | 1486 | CD2 | HIS | | 355 | −6.152 | 93.571 | −76.023 | 1.00 | 87.75 | |
| ATOM | 1487 | ND1 | HIS | | 355 | −7.600 | 95.012 | −76.801 | 1.00 | 87.65 | |
| ATOM | 1488 | CE1 | HIS | | 355 | −8.105 | 94.512 | −75.687 | 1.00 | 89.10 | |
| ATOM | 1489 | NE2 | HIS | | 355 | −7.246 | 93.637 | −75.194 | 1.00 | 89.32 | |
| ATOM | 1490 | C | HIS | | 355 | −4.057 | 96.050 | −76.560 | 1.00 | 80.62 | |
| ATOM | 1491 | O | HIS | | 355 | −4.595 | 96.378 | −75.504 | 1.00 | 79.87 | |
| ATOM | 1492 | N | GLU | | 356 | −2.841 | 95.520 | −76.618 | 1.00 | 79.66 | |
| ATOM | 1493 | CA | GLU | | 356 | −2.026 | 95.338 | −75.419 | 1.00 | 78.61 | |
| ATOM | 1494 | CB | GLU | | 356 | −0.847 | 94.420 | −75.722 | 1.00 | 80.80 | |
| ATOM | 1495 | CG | GLU | | 356 | −1.193 | 93.207 | −76.563 | 1.00 | 83.90 | |
| ATOM | 1496 | CD | GLU | | 356 | 0.001 | 92.724 | −77.378 | 1.00 | 86.67 | |
| ATOM | 1497 | OE1 | GLU | | 356 | 0.459 | 93.483 | −78.265 | 1.00 | 87.68 | |
| ATOM | 1498 | OE2 | GLU | | 356 | 0.489 | 91.597 | −77.132 | 1.00 | 87.85 | |
| ATOM | 1499 | C | GLU | | 356 | −1.505 | 96.714 | −75.015 | 1.00 | 76.92 | |
| ATOM | 1500 | O | GLU | | 356 | −0.868 | 96.877 | −73.969 | 1.00 | 75.86 | |
| ATOM | 1501 | N | ARG | | 357 | −1.793 | 97.694 | −75.873 | 1.00 | 74.27 | |
| ATOM | 1502 | CA | ARG | | 357 | −1.397 | 99.078 | −75.675 | 1.00 | 72.32 | |
| ATOM | 1503 | CB | ARG | | 357 | −1.191 | 99.739 | −77.037 | 1.00 | 74.34 | |
| ATOM | 1504 | CG | ARG | | 357 | 0.195 | 99.502 | −77.581 | 1.00 | 79.91 | |
| ATOM | 1505 | CD | ARG | | 357 | 0.263 | 99.418 | −79.105 | 1.00 | 82.36 | |
| ATOM | 1506 | NE | ARG | | 357 | 1.516 | 98.766 | −79.495 | 1.00 | 85.32 | |
| ATOM | 1507 | CZ | ARG | | 357 | 1.806 | 98.354 | −80.724 | 1.00 | 86.63 | |
| ATOM | 1508 | NH1 | ARG | | 357 | 0.932 | 98.530 | −81.707 | 1.00 | 87.74 | |
| ATOM | 1509 | NH2 | ARG | | 357 | 2.963 | 97.747 | −80.966 | 1.00 | 86.74 | |
| ATOM | 1510 | C | ARG | | 357 | −2.360 | 99.925 | −74.836 | 1.00 | 69.50 | |
| ATOM | 1511 | O | ARG | | 357 | −2.093 | 101.108 | −74.603 | 1.00 | 70.06 | |
| ATOM | 1512 | N | LEU | | 358 | −3.462 | 99.338 | −74.369 | 1.00 | 64.22 | |
| ATOM | 1513 | CA | LEU | | 358 | −4.426 | 100.101 | −73.573 | 1.00 | 60.12 | |
| ATOM | 1514 | CB | LEU | | 358 | −5.656 | 99.260 | −73.228 | 1.00 | 58.17 | |
| ATOM | 1515 | CG | LEU | | 358 | −6.603 | 99.058 | −74.406 | 1.00 | 55.93 | |
| ATOM | 1516 | CD1 | LEU | | 358 | −7.651 | 98.053 | −74.043 | 1.00 | 54.94 | |
| ATOM | 1517 | CD2 | LEU | | 358 | −7.227 | 100.391 | −74.799 | 1.00 | 56.22 | |
| ATOM | 1518 | C | LEU | | 358 | −3.847 | 100.675 | −72.302 | 1.00 | 58.03 | |
| ATOM | 1519 | O | LEU | | 358 | −4.130 | 101.821 | −71.971 | 1.00 | 57.79 | |
| ATOM | 1520 | N | PHE | | 359 | −3.034 | 99.898 | −71.588 | 1.00 | 55.72 | |
| ATOM | 1521 | CA | PHE | | 359 | −2.448 | 100.396 | −70.347 | 1.00 | 52.62 | |
| ATOM | 1522 | CB | PHE | | 359 | −1.434 | 99.407 | −69.769 | 1.00 | 47.27 | |
| ATOM | 1523 | CG | PHE | | 359 | −0.904 | 99.808 | −68.422 | 1.00 | 42.38 | |
| ATOM | 1524 | CD1 | PHE | | 359 | −1.547 | 99.405 | −67.257 | 1.00 | 38.30 | |
| ATOM | 1525 | CD2 | PHE | | 359 | 0.207 | 100.633 | −68.319 | 1.00 | 39.93 | |
| ATOM | 1526 | CE1 | PHE | | 359 | −1.099 | 99.815 | −66.011 | 1.00 | 39.00 | |
| ATOM | 1527 | CE2 | PHE | | 359 | 0.670 | 101.055 | −67.069 | 1.00 | 40.03 | |
| ATOM | 1528 | CZ | PHE | | 359 | 0.018 | 100.650 | −65.912 | 1.00 | 38.98 | |
| ATOM | 1529 | C | PHE | | 359 | −1.749 | 101.729 | −70.619 | 1.00 | 54.41 | |
| ATOM | 1530 | O | PHE | | 359 | −2.036 | 102.741 | −69.961 | 1.00 | 54.88 | |
| ATOM | 1531 | N | GLU | | 360 | −0.836 | 101.731 | −71.587 | 1.00 | 54.09 | |
| ATOM | 1532 | CA | GLU | | 360 | −0.116 | 102.952 | −71.931 | 1.00 | 54.89 | |
| ATOM | 1533 | CB | GLU | | 360 | 1.008 | 102.670 | −72.943 | 1.00 | 56.26 | |
| ATOM | 1534 | CG | GLU | | 360 | 1.838 | 101.412 | −72.700 | 1.00 | 59.95 | |
| ATOM | 1535 | CD | GLU | | 360 | 1.293 | 100.190 | −73.435 | 1.00 | 61.50 | |
| ATOM | 1536 | OE1 | GLU | | 360 | 0.748 | 99.292 | −72.760 | 1.00 | 64.26 | |
| ATOM | 1537 | OE2 | GLU | | 360 | 1.401 | 100.125 | −74.683 | 1.00 | 60.63 | |
| ATOM | 1538 | C | GLU | | 360 | −1.079 | 103.993 | −72.541 | 1.00 | 53.30 | |
| ATOM | 1539 | O | GLU | | 360 | −0.979 | 105.186 | −72.265 | 1.00 | 53.36 | |
| ATOM | 1540 | N | LEU | | 361 | −2.004 | 103.533 | −73.371 | 1.00 | 50.46 | |
| ATOM | 1541 | CA | LEU | | 361 | −2.941 | 104.430 | −74.024 | 1.00 | 49.32 | |
| ATOM | 1542 | CB | LEU | | 361 | −3.773 | 103.655 | −75.044 | 1.00 | 49.29 | |
| ATOM | 1543 | CG | LEU | | 361 | −3.043 | 103.239 | −76.317 | 1.00 | 49.37 | |
| ATOM | 1544 | CD1 | LEU | | 361 | −4.047 | 102.750 | −77.322 | 1.00 | 49.11 | |
| ATOM | 1545 | CD2 | LEU | | 361 | −2.290 | 104.440 | −76.891 | 1.00 | 51.54 | |
| ATOM | 1546 | C | LEU | | 361 | −3.874 | 105.190 | −73.079 | 1.00 | 48.48 | |
| ATOM | 1547 | O | LEU | | 361 | −3.886 | 106.423 | −73.059 | 1.00 | 46.65 | |
| ATOM | 1548 | N | ILE | | 362 | −4.657 | 104.443 | −72.310 | 1.00 | 46.31 | |
| ATOM | 1549 | CA | ILE | | 362 | −5.590 | 105.027 | −71.368 | 1.00 | 45.23 | |
| ATOM | 1550 | CB | ILE | | 362 | −6.303 | 103.938 | −70.556 | 1.00 | 43.00 | |
| ATOM | 1551 | CG2 | ILE | | 362 | −7.225 | 104.568 | −69.518 | 1.00 | 41.69 | |
| ATOM | 1552 | CG1 | ILE | | 362 | −7.096 | 103.049 | −71.500 | 1.00 | 42.41 | |
| ATOM | 1553 | CD1 | ILE | | 362 | −7.636 | 101.818 | −70.838 | 1.00 | 42.87 | |
| ATOM | 1554 | C | ILE | | 362 | −4.899 | 105.970 | −70.406 | 1.00 | 45.61 | |
| ATOM | 1555 | O | ILE | | 362 | −5.489 | 106.937 | −69.945 | 1.00 | 46.19 | |
| ATOM | 1556 | N | LEU | | 363 | −3.644 | 105.701 | −70.108 | 1.00 | 47.28 | |
| ATOM | 1557 | CA | LEU | | 363 | −2.926 | 106.538 | −69.174 | 1.00 | 50.66 | |
| ATOM | 1558 | CB | LEU | | 363 | −1.884 | 105.721 | −68.432 | 1.00 | 50.05 | |
| ATOM | 1559 | CG | LEU | | 363 | −2.428 | 104.872 | −67.298 | 1.00 | 51.81 | |
| ATOM | 1560 | CD1 | LEU | | 363 | −1.435 | 103.757 | −66.994 | 1.00 | 49.66 | |
| ATOM | 1561 | CD2 | LEU | | 363 | −2.671 | 105.750 | −66.077 | 1.00 | 50.15 | |
| ATOM | 1562 | C | LEU | | 363 | −2.241 | 107.743 | −69.757 | 1.00 | 52.43 | |
| ATOM | 1563 | O | LEU | | 363 | −2.322 | 108.813 | −69.177 | 1.00 | 53.85 | |
| ATOM | 1564 | N | MET | | 364 | −1.581 | 107.580 | −70.899 | 1.00 | 53.89 | |
| ATOM | 1565 | CA | MET | | 364 | −0.815 | 108.671 | −71.494 | 1.00 | 56.15 | |
| ATOM | 1566 | CB | MET | | 364 | 0.668 | 108.311 | −71.453 | 1.00 | 56.17 | |
| ATOM | 1567 | CG | MET | | 364 | 1.154 | 107.902 | −70.088 | 1.00 | 58.72 | |
| ATOM | 1568 | SD | MET | | 364 | 2.845 | 107.242 | −70.080 | 1.00 | 61.25 | |
| ATOM | 1569 | CE | MET | | 364 | 2.525 | 105.577 | −70.470 | 1.00 | 59.93 | |
| ATOM | 1570 | C | MET | | 364 | −1.159 | 109.093 | −72.910 | 1.00 | 56.72 | |
| ATOM | 1571 | O | MET | | 364 | −0.388 | 109.797 | −73.542 | 1.00 | 58.49 | |
| ATOM | 1572 | N | GLU | | 365 | −2.299 | 108.682 | −73.421 | 1.00 | 57.45 | |
| ATOM | 1573 | CA | GLU | | 365 | −2.654 | 109.048 | −74.783 | 1.00 | 58.82 | |
| ATOM | 1574 | CB | GLU | | 365 | −2.927 | 107.767 | −75.574 | 1.00 | 60.59 | |
| ATOM | 1575 | CG | GLU | | 365 | −3.351 | 107.998 | −76.995 | 1.00 | 62.48 | |
| ATOM | 1576 | CD | GLU | | 365 | −2.242 | 108.579 | −77.814 | 1.00 | 64.91 | |
| ATOM | 1577 | OE1 | GLU | | 365 | −1.669 | 109.608 | −77.386 | 1.00 | 65.42 | |
| ATOM | 1578 | OE2 | GLU | | 365 | −1.940 | 108.003 | −78.882 | 1.00 | 66.95 | |
| ATOM | 1579 | C | GLU | | 365 | −3.877 | 109.980 | −74.866 | 1.00 | 59.32 | |
| ATOM | 1580 | O | GLU | | 365 | −4.943 | 109.652 | −74.336 | 1.00 | 58.16 | |
| ATOM | 1581 | N | GLU | | 366 | −3.740 | 111.128 | −75.531 | 1.00 | 60.08 | |
| ATOM | 1582 | CA | GLU | | 366 | −4.885 | 112.034 | −75.655 | 1.00 | 61.95 | |
| ATOM | 1583 | CB | GLU | | 366 | −4.514 | 113.327 | −76.389 | 1.00 | 65.27 | |
| ATOM | 1584 | CG | GLU | | 366 | −4.218 | 113.143 | −77.890 | 1.00 | 70.51 | |
| ATOM | 1585 | CD | GLU | | 366 | −4.067 | 114.469 | −78.658 | 1.00 | 72.07 | |
| ATOM | 1586 | OE1 | GLU | | 366 | −5.100 | 115.096 | −78.995 | 1.00 | 72.95 | |
| ATOM | 1587 | OE2 | GLU | | 366 | −2.912 | 114.880 | −78.918 | 1.00 | 72.96 | |
| ATOM | 1588 | C | GLU | | 366 | −5.965 | 111.308 | −76.444 | 1.00 | 60.79 | |
| ATOM | 1589 | O | GLU | | 366 | −5.682 | 110.679 | −77.466 | 1.00 | 61.20 | |
| ATOM | 1590 | N | ILE | | 367 | −7.193 | 111.382 | −75.949 | 1.00 | 58.81 | |
| ATOM | 1591 | CA | ILE | | 367 | −8.326 | 110.740 | −76.594 | 1.00 | 59.29 | |
| ATOM | 1592 | CB | ILE | | 367 | −9.436 | 110.401 | −75.557 | 1.00 | 58.55 | |
| ATOM | 1593 | CG2 | ILE | | 367 | −10.740 | 110.094 | −76.259 | 1.00 | 57.35 | |
| ATOM | 1594 | CG1 | ILE | | 367 | −8.989 | 109.216 | −74.691 | 1.00 | 58.42 | |
| ATOM | 1595 | CD1 | ILE | | 367 | −10.002 | 108.764 | −73.650 | 1.00 | 56.35 | |
| ATOM | 1596 | C | ILE | | 367 | −8.866 | 111.695 | −77.656 | 1.00 | 60.85 | |
| ATOM | 1597 | O | ILE | | 367 | −9.357 | 112.786 | −77.346 | 1.00 | 61.82 | |
| ATOM | 1598 | N | ARG | | 368 | −8.768 | 111.268 | −78.910 | 1.00 | 61.14 | |
| ATOM | 1599 | CA | ARG | | 368 | −9.204 | 112.053 | −80.060 | 1.00 | 60.62 | |
| ATOM | 1600 | CB | ARG | | 368 | −8.402 | 111.582 | −81.290 | 1.00 | 62.52 | |
| ATOM | 1601 | CG | ARG | | 368 | −8.695 | 112.294 | −82.587 | 1.00 | 68.23 | |
| ATOM | 1602 | CD | ARG | | 368 | −7.459 | 112.359 | −83.502 | 1.00 | 72.52 | |
| ATOM | 1603 | NE | ARG | | 368 | −6.505 | 113.424 | −83.147 | 1.00 | 75.13 | |
| ATOM | 1604 | CZ | ARG | | 368 | −6.734 | 114.740 | −83.254 | 1.00 | 76.98 | |
| ATOM | 1605 | NH1 | ARG | | 368 | −7.901 | 115.210 | −83.709 | 1.00 | 75.68 | |
| ATOM | 1606 | NH2 | ARG | | 368 | −5.779 | 115.598 | −82.912 | 1.00 | 77.83 | |
| ATOM | 1607 | C | ARG | | 368 | −10.716 | 111.927 | −80.280 | 1.00 | 59.50 | |
| ATOM | 1608 | O | ARG | | 368 | −11.290 | 110.876 | −80.011 | 1.00 | 57.56 | |
| ATOM | 1609 | N | PHE | | 369 | −11.349 | 113.006 | −80.761 | 1.00 | 59.17 | |
| ATOM | 1610 | CA | PHE | | 369 | −12.804 | 113.052 | −81.010 | 1.00 | 57.48 | |
| ATOM | 1611 | CB | PHE | | 369 | −13.452 | 114.164 | −80.175 | 1.00 | 55.03 | |
| ATOM | 1612 | CG | PHE | | 369 | −13.170 | 114.067 | −78.723 | 1.00 | 50.65 | |
| ATOM | 1613 | CD1 | PHE | | 369 | −13.747 | 113.070 | −77.958 | 1.00 | 49.85 | |
| ATOM | 1614 | CD2 | PHE | | 369 | −12.291 | 114.950 | −78.125 | 1.00 | 51.15 | |
| ATOM | 1615 | CE1 | PHE | | 369 | −13.444 | 112.951 | −76.620 | 1.00 | 50.03 | |
| ATOM | 1616 | CE2 | PHE | | 369 | −11.980 | 114.839 | −76.782 | 1.00 | 51.20 | |
| ATOM | 1617 | CZ | PHE | | 369 | −12.558 | 113.840 | −76.028 | 1.00 | 50.48 | |
| ATOM | 1618 | C | PHE | | 369 | −13.191 | 113.310 | −82.466 | 1.00 | 57.86 | |
| ATOM | 1619 | O | PHE | | 369 | −12.643 | 114.207 | −83.109 | 1.00 | 58.06 | |
| ATOM | 1620 | N | PRO | | 370 | −14.161 | 112.549 | −82.997 | 1.00 | 57.46 | |
| ATOM | 1621 | CD | PRO | | 370 | −14.923 | 111.456 | −82.380 | 1.00 | 57.08 | |
| ATOM | 1622 | CA | PRO | | 370 | −14.578 | 112.758 | −84.381 | 1.00 | 58.74 | |
| ATOM | 1623 | CB | PRO | | 370 | −15.851 | 111.930 | −84.479 | 1.00 | 56.87 | |
| ATOM | 1624 | CG | PRO | | 370 | −15.597 | 110.815 | −83.562 | 1.00 | 56.30 | |
| ATOM | 1625 | C | PRO | | 370 | −14.837 | 114.252 | −84.600 | 1.00 | 61.42 | |
| ATOM | 1626 | O | PRO | | 370 | −15.027 | 114.996 | −83.633 | 1.00 | 60.59 | |
| ATOM | 1627 | N | ARG | | 371 | −14.829 | 114.694 | −85.860 | 1.00 | 63.84 | |
| ATOM | 1628 | CA | ARG | | 371 | −15.067 | 116.103 | −86.166 | 1.00 | 65.67 | |
| ATOM | 1629 | CB | ARG | | 371 | −14.529 | 116.464 | −87.567 | 1.00 | 68.73 | |
| ATOM | 1630 | CG | ARG | | 371 | −13.042 | 116.127 | −87.862 | 1.00 | 70.85 | |
| ATOM | 1631 | CD | ARG | | 371 | −12.645 | 116.430 | −89.354 | 1.00 | 73.12 | |
| ATOM | 1632 | NE | ARG | | 371 | −13.489 | 115.727 | −90.341 | 1.00 | 75.75 | |
| ATOM | 1633 | CZ | ARG | | 371 | −13.244 | 114.522 | −90.870 | 1.00 | 76.26 | |
| ATOM | 1634 | NH1 | ARG | | 371 | −12.153 | 113.835 | −90.543 | 1.00 | 77.37 | |
| ATOM | 1635 | NH2 | ARG | | 371 | −14.120 | 113.978 | −91.708 | 1.00 | 75.59 | |
| ATOM | 1636 | C | ARG | | 371 | −16.585 | 116.329 | −86.113 | 1.00 | 65.13 | |
| ATOM | 1637 | O | ARG | | 371 | −17.051 | 117.381 | −85.713 | 1.00 | 64.96 | |
| ATOM | 1638 | N | THR | | 372 | −17.340 | 115.314 | −86.505 | 1.00 | 64.83 | |
| ATOM | 1639 | CA | THR | | 372 | −18.796 | 115.362 | −86.517 | 1.00 | 66.68 | |
| ATOM | 1640 | CB | THR | | 372 | −19.317 | 114.141 | −87.307 | 1.00 | 66.65 | |
| ATOM | 1641 | OG1 | THR | | 372 | −20.589 | 113.722 | −86.800 | 1.00 | 66.63 | |
| ATOM | 1642 | CG2 | THR | | 372 | −18.316 | 112.984 | −87.205 | 1.00 | 68.01 | |
| ATOM | 1643 | C | THR | | 372 | −19.458 | 115.402 | −85.115 | 1.00 | 68.74 | |
| ATOM | 1644 | O | THR | | 372 | −20.558 | 114.862 | −84.912 | 1.00 | 69.22 | |
| ATOM | 1645 | N | LEU | | 373 | −18.812 | 116.058 | −84.151 | 1.00 | 68.80 | |
| ATOM | 1646 | CA | LEU | | 373 | −19.349 | 116.103 | −82.786 | 1.00 | 67.20 | |
| ATOM | 1647 | CB | LEU | | 373 | −18.308 | 115.549 | −81.805 | 1.00 | 67.75 | |
| ATOM | 1648 | CG | LEU | | 373 | −18.834 | 114.570 | −80.754 | 1.00 | 67.05 | |
| ATOM | 1649 | CD1 | LEU | | 373 | −19.565 | 113.431 | −81.463 | 1.00 | 66.30 | |
| ATOM | 1650 | CD2 | LEU | | 373 | −17.700 | 114.043 | −79.905 | 1.00 | 66.07 | |
| ATOM | 1651 | C | LEU | | 373 | −19.754 | 117.491 | −82.339 | 1.00 | 66.05 | |
| ATOM | 1652 | O | LEU | | 373 | −18.989 | 118.434 | −82.505 | 1.00 | 64.33 | |
| ATOM | 1653 | N | SER | | 374 | −20.948 | 117.609 | −81.760 | 1.00 | 65.94 | |
| ATOM | 1654 | CA | SER | | 374 | −21.437 | 118.903 | −81.281 | 1.00 | 66.63 | |
| ATOM | 1655 | CB | SER | | 374 | −22.794 | 118.740 | −80.612 | 1.00 | 66.82 | |
| ATOM | 1656 | OG | SER | | 374 | −22.640 | 118.150 | −79.329 | 1.00 | 68.04 | |
| ATOM | 1657 | C | SER | | 374 | −20.435 | 119.443 | −80.259 | 1.00 | 66.46 | |
| ATOM | 1658 | O | SER | | 374 | −20.113 | 118.771 | −79.291 | 1.00 | 67.55 | |
| ATOM | 1659 | N | PRO | | 375 | −19.964 | 120.681 | −80.439 | 1.00 | 66.35 | |
| ATOM | 1660 | CD | PRO | | 375 | −20.566 | 121.710 | −81.304 | 1.00 | 65.88 | |
| ATOM | 1661 | CA | PRO | | 375 | −18.985 | 121.271 | −79.509 | 1.00 | 65.76 | |
| ATOM | 1662 | CB | PRO | | 375 | −19.020 | 122.761 | −79.863 | 1.00 | 65.81 | |
| ATOM | 1663 | CG | PRO | | 375 | −20.407 | 122.944 | −80.470 | 1.00 | 66.57 | |
| ATOM | 1664 | C | PRO | | 375 | −19.214 | 120.995 | −78.020 | 1.00 | 65.51 | |
| ATOM | 1665 | O | PRO | | 375 | −18.270 | 121.022 | −77.221 | 1.00 | 64.18 | |
| ATOM | 1666 | N | GLU | | 376 | −20.465 | 120.730 | −77.655 | 1.00 | 65.60 | |
| ATOM | 1667 | CA | GLU | | 376 | −20.807 | 120.427 | −76.268 | 1.00 | 65.58 | |
| ATOM | 1668 | CB | GLU | | 376 | −22.299 | 120.682 | −75.995 | 1.00 | 64.87 | |
| ATOM | 1669 | CG | GLU | | 376 | −23.240 | 120.197 | −77.079 | 1.00 | 67.54 | |
| ATOM | 1670 | CD | GLU | | 376 | −23.425 | 121.213 | −78.208 | 1.00 | 69.22 | |
| ATOM | 1671 | OE1 | GLU | | 376 | −22.465 | 121.962 | −78.522 | 1.00 | 69.87 | |
| ATOM | 1672 | OE2 | GLU | | 376 | −24.530 | 121.249 | −78.794 | 1.00 | 67.83 | |
| ATOM | 1673 | C | GLU | | 376 | −20.451 | 118.964 | −75.950 | 1.00 | 64.54 | |
| ATOM | 1674 | O | GLU | | 376 | −19.781 | 118.695 | −74.932 | 1.00 | 64.14 | |
| ATOM | 1675 | N | ALA | | 377 | −20.880 | 118.035 | −76.817 | 1.00 | 61.55 | |
| ATOM | 1676 | CA | ALA | | 377 | −20.578 | 116.611 | −76.629 | 1.00 | 59.15 | |
| ATOM | 1677 | CB | ALA | | 377 | −21.120 | 115.793 | −77.786 | 1.00 | 58.55 | |
| ATOM | 1678 | C | ALA | | 377 | −19.067 | 116.435 | −76.512 | 1.00 | 56.95 | |
| ATOM | 1679 | O | ALA | | 377 | −18.576 | 115.721 | −75.646 | 1.00 | 56.48 | |
| ATOM | 1680 | N | LYS | | 378 | −18.332 | 117.119 | −77.374 | 1.00 | 56.10 | |
| ATOM | 1681 | CA | LYS | | 378 | −16.882 | 117.063 | −77.347 | 1.00 | 57.07 | |
| ATOM | 1682 | CB | LYS | | 378 | −16.307 | 117.800 | −78.567 | 1.00 | 59.37 | |
| ATOM | 1683 | CG | LYS | | 378 | −14.766 | 117.884 | −78.630 | 1.00 | 62.85 | |
| ATOM | 1684 | CD | LYS | | 378 | −14.298 | 118.366 | −80.020 | 1.00 | 64.41 | |
| ATOM | 1685 | CE | LYS | | 378 | −12.782 | 118.471 | −80.126 | 1.00 | 66.49 | |
| ATOM | 1686 | NZ | LYS | | 378 | −12.305 | 118.762 | −81.519 | 1.00 | 66.73 | |
| ATOM | 1687 | C | LYS | | 378 | −16.347 | 117.663 | −76.051 | 1.00 | 55.38 | |
| ATOM | 1688 | O | LYS | | 378 | −15.250 | 117.308 | −75.592 | 1.00 | 55.74 | |
| ATOM | 1689 | N | SER | | 379 | −17.125 | 118.566 | −75.457 | 1.00 | 54.04 | |
| ATOM | 1690 | CA | SER | | 379 | −16.728 | 119.212 | −74.198 | 1.00 | 51.41 | |
| ATOM | 1691 | CB | SER | | 379 | −17.551 | 120.496 | −73.934 | 1.00 | 52.13 | |
| ATOM | 1692 | OG | SER | | 379 | −16.845 | 121.401 | −73.069 | 1.00 | 50.56 | |
| ATOM | 1693 | C | SER | | 379 | −16.964 | 118.221 | −73.067 | 1.00 | 49.05 | |
| ATOM | 1694 | O | SER | | 379 | −16.132 | 118.073 | −72.169 | 1.00 | 47.48 | |
| ATOM | 1695 | N | LEU | | 380 | −18.111 | 117.551 | −73.143 | 1.00 | 45.28 | |
| ATOM | 1696 | CA | LEU | | 380 | −18.497 | 116.564 | −72.167 | 1.00 | 43.11 | |
| ATOM | 1697 | CB | LEU | | 380 | −19.878 | 116.017 | −72.526 | 1.00 | 38.76 | |
| ATOM | 1698 | CG | LEU | | 380 | −20.533 | 115.044 | −71.528 | 1.00 | 39.57 | |
| ATOM | 1699 | CD1 | LEU | | 380 | −20.838 | 115.719 | −70.185 | 1.00 | 37.26 | |
| ATOM | 1700 | CD2 | LEU | | 380 | −21.806 | 114.513 | −72.114 | 1.00 | 34.86 | |
| ATOM | 1701 | C | LEU | | 380 | −17.436 | 115.429 | −72.144 | 1.00 | 44.07 | |
| ATOM | 1702 | O | LEU | | 380 | −16.745 | 115.192 | −71.128 | 1.00 | 40.04 | |
| ATOM | 1703 | N | LEU | | 381 | −17.309 | 114.736 | −73.266 | 1.00 | 44.99 | |
| ATOM | 1704 | CA | LEU | | 381 | −16.356 | 113.647 | −73.349 | 1.00 | 46.41 | |
| ATOM | 1705 | CB | LEU | | 381 | −16.247 | 113.161 | −74.779 | 1.00 | 48.06 | |
| ATOM | 1706 | CG | LEU | | 381 | −17.641 | 112.708 | −75.191 | 1.00 | 49.42 | |
| ATOM | 1707 | CD1 | LEU | | 381 | −17.711 | 112.436 | −76.691 | 1.00 | 48.99 | |
| ATOM | 1708 | CD2 | LEU | | 381 | −17.980 | 111.495 | −74.381 | 1.00 | 48.20 | |
| ATOM | 1709 | C | LEU | | 381 | −15.011 | 114.079 | −72.834 | 1.00 | 45.89 | |
| ATOM | 1710 | O | LEU | | 381 | −14.420 | 113.404 | −72.008 | 1.00 | 47.29 | |
| ATOM | 1711 | N | ALA | | 382 | −14.519 | 115.217 | −73.285 | 1.00 | 45.76 | |
| ATOM | 1712 | CA | ALA | | 382 | −13.220 | 115.632 | −72.793 | 1.00 | 46.22 | |
| ATOM | 1713 | CB | ALA | | 382 | −12.815 | 116.967 | −73.425 | 1.00 | 45.57 | |
| ATOM | 1714 | C | ALA | | 382 | −13.241 | 115.731 | −71.263 | 1.00 | 46.30 | |
| ATOM | 1715 | O | ALA | | 382 | −12.266 | 115.396 | −70.594 | 1.00 | 46.70 | |
| ATOM | 1716 | N | GLY | | 383 | −14.363 | 116.183 | −70.710 | 1.00 | 47.25 | |
| ATOM | 1717 | CA | GLY | | 383 | −14.465 | 116.312 | −69.263 | 1.00 | 46.46 | |
| ATOM | 1718 | C | GLY | | 383 | −14.568 | 114.980 | −68.527 | 1.00 | 45.14 | |
| ATOM | 1719 | O | GLY | | 383 | −13.926 | 114.776 | −67.501 | 1.00 | 43.56 | |
| ATOM | 1720 | N | LEU | | 384 | −15.384 | 114.075 | −69.050 | 1.00 | 43.14 | |
| ATOM | 1721 | CA | LEU | | 384 | −15.541 | 112.788 | −68.432 | 1.00 | 44.46 | |
| ATOM | 1722 | CB | LEU | | 384 | −16.736 | 112.078 | −69.038 | 1.00 | 41.26 | |
| ATOM | 1723 | CG | LEU | | 384 | −18.061 | 112.778 | −68.723 | 1.00 | 40.91 | |
| ATOM | 1724 | CD1 | LEU | | 384 | −19.241 | 111.945 | −69.258 | 1.00 | 36.77 | |
| ATOM | 1725 | CD2 | LEU | | 384 | −18.168 | 112.974 | −67.188 | 1.00 | 39.38 | |
| ATOM | 1726 | C | LEU | | 384 | −14.294 | 111.928 | −68.593 | 1.00 | 47.06 | |
| ATOM | 1727 | O | LEU | | 384 | −14.144 | 110.902 | −67.924 | 1.00 | 50.25 | |
| ATOM | 1728 | N | LEU | | 385 | −13.381 | 112.351 | −69.459 | 1.00 | 46.08 | |
| ATOM | 1729 | CA | LEU | | 385 | −12.193 | 111.565 | −69.708 | 1.00 | 43.78 | |
| ATOM | 1730 | CB | LEU | | 385 | −12.124 | 111.220 | −71.171 | 1.00 | 40.79 | |
| ATOM | 1731 | CG | LEU | | 385 | −13.379 | 110.487 | −71.586 | 1.00 | 41.49 | |
| ATOM | 1732 | CD1 | LEU | | 385 | −13.433 | 110.335 | −73.108 | 1.00 | 42.27 | |
| ATOM | 1733 | CD2 | LEU | | 385 | −13.395 | 109.163 | −70.886 | 1.00 | 42.15 | |
| ATOM | 1734 | C | LEU | | 385 | −10.885 | 112.173 | −69.281 | 1.00 | 45.19 | |
| ATOM | 1735 | O | LEU | | 385 | −9.840 | 111.689 | −69.699 | 1.00 | 44.34 | |
| ATOM | 1736 | N | LYS | | 386 | −10.914 | 113.224 | −68.468 | 1.00 | 44.93 | |
| ATOM | 1737 | CA | LYS | | 386 | −9.661 | 113.789 | −68.005 | 1.00 | 48.68 | |
| ATOM | 1738 | CB | LYS | | 386 | −9.910 | 114.978 | −67.070 | 1.00 | 50.26 | |
| ATOM | 1739 | CG | LYS | | 386 | −10.478 | 116.202 | −67.757 | 1.00 | 54.40 | |
| ATOM | 1740 | CD | LYS | | 386 | −9.449 | 116.873 | −68.663 | 1.00 | 55.80 | |
| ATOM | 1741 | CE | LYS | | 386 | −8.889 | 118.178 | −68.061 | 1.00 | 59.30 | |
| ATOM | 1742 | NZ | LYS | | 386 | −8.176 | 118.012 | −66.747 | 1.00 | 59.24 | |
| ATOM | 1743 | C | LYS | | 386 | −8.954 | 112.644 | −67.258 | 1.00 | 50.22 | |
| ATOM | 1744 | O | LYS | | 386 | −9.555 | 111.985 | −66.418 | 1.00 | 49.08 | |
| ATOM | 1745 | N | LYS | | 387 | −7.686 | 112.405 | −67.575 | 1.00 | 51.70 | |
| ATOM | 1746 | CA | LYS | | 387 | −6.939 | 111.329 | −66.940 | 1.00 | 54.32 | |
| ATOM | 1747 | CB | LYS | | 387 | −5.638 | 111.096 | −67.715 | 1.00 | 53.52 | |
| ATOM | 1748 | CG | LYS | | 387 | −5.908 | 110.577 | −69.121 | 1.00 | 53.08 | |
| ATOM | 1749 | CD | LYS | | 387 | −4.667 | 110.602 | −69.989 | 1.00 | 52.12 | |
| ATOM | 1750 | CE | LYS | | 387 | −4.637 | 109.426 | −70.975 | 1.00 | 51.71 | |
| ATOM | 1751 | NZ | LYS | | 387 | −5.836 | 109.281 | −71.860 | 1.00 | 47.59 | |
| ATOM | 1752 | C | LYS | | 387 | −6.670 | 111.596 | −65.461 | 1.00 | 55.74 | |
| ATOM | 1753 | O | LYS | | 387 | −6.152 | 110.748 | −64.742 | 1.00 | 55.94 | |
| ATOM | 1754 | N | ASP | | 388 | −7.041 | 112.783 | −65.006 | 1.00 | 57.24 | |
| ATOM | 1755 | CA | ASP | | 388 | −6.857 | 113.155 | −63.608 | 1.00 | 59.65 | |
| ATOM | 1756 | CB | ASP | | 388 | −6.310 | 114.576 | −63.523 | 1.00 | 59.36 | |
| ATOM | 1757 | CG | ASP | | 388 | −5.664 | 114.873 | −62.198 | 1.00 | 60.90 | |
| ATOM | 1758 | OD1 | ASP | | 388 | −6.186 | 114.405 | −61.164 | 1.00 | 62.09 | |
| ATOM | 1759 | OD2 | ASP | | 388 | −4.634 | 115.588 | −62.189 | 1.00 | 61.76 | |
| ATOM | 1760 | C | ASP | | 388 | −8.238 | 113.098 | −62.946 | 1.00 | 61.18 | |
| ATOM | 1761 | O | ASP | | 388 | −9.153 | 113.822 | −63.353 | 1.00 | 61.70 | |
| ATOM | 1762 | N | PRO | | 389 | −8.426 | 112.229 | −61.932 | 1.00 | 61.74 | |
| ATOM | 1763 | CD | PRO | | 389 | −7.562 | 111.229 | −61.278 | 1.00 | 61.36 | |
| ATOM | 1764 | CA | PRO | | 389 | −9.776 | 112.235 | −61.348 | 1.00 | 61.60 | |
| ATOM | 1765 | CB | PRO | | 389 | −9.720 | 111.105 | −60.314 | 1.00 | 61.15 | |
| ATOM | 1766 | CG | PRO | | 389 | −8.258 | 111.024 | −59.954 | 1.00 | 60.71 | |
| ATOM | 1767 | C | PRO | | 389 | −10.104 | 113.602 | −60.746 | 1.00 | 61.45 | |
| ATOM | 1768 | O | PRO | | 389 | −11.205 | 114.116 | −60.914 | 1.00 | 61.30 | |
| ATOM | 1769 | N | LYS | | 390 | −9.122 | 114.201 | −60.083 | 1.00 | 61.86 | |
| ATOM | 1770 | CA | LYS | | 390 | −9.290 | 115.501 | −59.462 | 1.00 | 61.85 | |
| ATOM | 1771 | CB | LYS | | 390 | −7.967 | 115.944 | −58.824 | 1.00 | 62.78 | |
| ATOM | 1772 | CG | LYS | | 390 | −7.835 | 115.572 | −57.343 | 1.00 | 65.33 | |
| ATOM | 1773 | CD | LYS | | 390 | −6.398 | 115.721 | −56.844 | 1.00 | 69.08 | |
| ATOM | 1774 | CE | LYS | | 390 | −6.316 | 116.072 | −55.348 | 1.00 | 73.04 | |
| ATOM | 1775 | NZ | LYS | | 390 | −6.920 | 115.057 | −54.412 | 1.00 | 75.04 | |
| ATOM | 1776 | C | LYS | | 390 | −9.783 | 116.547 | −60.453 | 1.00 | 61.37 | |
| ATOM | 1777 | O | LYS | | 390 | −10.512 | 117.472 | −60.082 | 1.00 | 61.57 | |
| ATOM | 1778 | N | GLN | | 391 | −9.404 | 116.400 | −61.715 | 1.00 | 60.55 | |
| ATOM | 1779 | CA | GLN | | 391 | −9.830 | 117.358 | −62.729 | 1.00 | 59.17 | |
| ATOM | 1780 | CB | GLN | | 391 | −8.670 | 117.734 | −63.641 | 1.00 | 61.43 | |
| ATOM | 1781 | CG | GLN | | 391 | −7.491 | 118.394 | −62.966 | 1.00 | 66.61 | |
| ATOM | 1782 | CD | GLN | | 391 | −6.344 | 118.587 | −63.946 | 1.00 | 71.01 | |
| ATOM | 1783 | OE1 | GLN | | 391 | −6.492 | 119.260 | −64.980 | 1.00 | 72.45 | |
| ATOM | 1784 | NE2 | GLN | | 391 | −5.193 | 117.982 | −63.639 | 1.00 | 73.41 | |
| ATOM | 1785 | C | GLN | | 391 | −10.961 | 116.851 | −63.600 | 1.00 | 57.21 | |
| ATOM | 1786 | O | GLN | | 391 | −11.419 | 117.562 | −64.484 | 1.00 | 59.18 | |
| ATOM | 1787 | N | ARG | | 392 | −11.426 | 115.633 | −63.367 | 1.00 | 54.41 | |
| ATOM | 1788 | CA | ARG | | 392 | −12.491 | 115.096 | −64.203 | 1.00 | 52.12 | |
| ATOM | 1789 | CB | ARG | | 392 | −12.660 | 113.599 | −63.937 | 1.00 | 51.91 | |
| ATOM | 1790 | CG | ARG | | 392 | −13.469 | 112.810 | −64.973 | 1.00 | 50.23 | |
| ATOM | 1791 | CD | ARG | | 392 | −13.170 | 111.289 | −64.814 | 1.00 | 52.15 | |
| ATOM | 1792 | NE | ARG | | 392 | −11.727 | 111.045 | −64.788 | 1.00 | 51.60 | |
| ATOM | 1793 | CZ | ARG | | 392 | −11.127 | 110.060 | −64.127 | 1.00 | 51.35 | |
| ATOM | 1794 | NH1 | ARG | | 392 | −11.850 | 109.194 | −63.431 | 1.00 | 51.78 | |
| ATOM | 1795 | NH2 | ARG | | 392 | −9.794 | 109.980 | −64.121 | 1.00 | 49.46 | |
| ATOM | 1796 | C | ARG | | 392 | −13.823 | 115.798 | −64.008 | 1.00 | 51.09 | |
| ATOM | 1797 | O | ARG | | 392 | −14.121 | 116.328 | −62.947 | 1.00 | 50.99 | |
| ATOM | 1798 | N | LEU | | 393 | −14.614 | 115.811 | −65.060 | 1.00 | 49.90 | |
| ATOM | 1799 | CA | LEU | | 393 | −15.924 | 116.386 | −64.989 | 1.00 | 50.20 | |
| ATOM | 1800 | CB | LEU | | 393 | −16.583 | 116.304 | −66.362 | 1.00 | 47.21 | |
| ATOM | 1801 | CG | LEU | | 393 | −17.757 | 117.184 | −66.789 | 1.00 | 46.02 | |
| ATOM | 1802 | CD1 | LEU | | 393 | −18.598 | 116.420 | −67.792 | 1.00 | 45.59 | |
| ATOM | 1803 | CD2 | LEU | | 393 | −18.593 | 117.562 | −65.617 | 1.00 | 48.67 | |
| ATOM | 1804 | C | LEU | | 393 | −16.630 | 115.436 | −64.012 | 1.00 | 52.76 | |
| ATOM | 1805 | O | LEU | | 393 | −16.912 | 114.285 | −64.354 | 1.00 | 53.97 | |
| ATOM | 1806 | N | GLY | | 394 | −16.903 | 115.902 | −62.796 | 1.00 | 53.46 | |
| ATOM | 1807 | CA | GLY | | 394 | −17.576 | 115.055 | −61.826 | 1.00 | 52.91 | |
| ATOM | 1808 | C | GLY | | 394 | −16.643 | 114.595 | −60.728 | 1.00 | 52.32 | |
| ATOM | 1809 | O | GLY | | 394 | −17.047 | 113.925 | −59.780 | 1.00 | 50.61 | |
| ATOM | 1810 | N | GLY | | 395 | −15.381 | 114.969 | −60.872 | 1.00 | 53.78 | |
| ATOM | 1811 | CA | GLY | | 395 | −14.374 | 114.592 | −59.906 | 1.00 | 55.17 | |
| ATOM | 1812 | C | GLY | | 395 | −14.268 | 115.729 | −58.935 | 1.00 | 56.78 | |
| ATOM | 1813 | O | GLY | | 395 | −13.474 | 115.705 | −57.987 | 1.00 | 57.28 | |
| ATOM | 1814 | N | GLY | | 396 | −15.085 | 116.741 | −59.188 | 1.00 | 56.35 | |
| ATOM | 1815 | CA | GLY | | 396 | −15.101 | 117.898 | −58.320 | 1.00 | 56.73 | |
| ATOM | 1816 | C | GLY | | 396 | −15.861 | 117.602 | −57.042 | 1.00 | 56.84 | |
| ATOM | 1817 | O | GLY | | 396 | −16.788 | 116.787 | −57.038 | 1.00 | 53.97 | |
| ATOM | 1818 | N | PRO | | 397 | −15.495 | 118.272 | −55.940 | 1.00 | 57.58 | |
| ATOM | 1819 | CD | PRO | | 397 | −14.603 | 119.442 | −55.934 | 1.00 | 57.97 | |
| ATOM | 1820 | CA | PRO | | 397 | −16.123 | 118.103 | −54.628 | 1.00 | 58.12 | |
| ATOM | 1821 | CB | PRO | | 397 | −15.501 | 119.228 | −53.801 | 1.00 | 59.18 | |
| ATOM | 1822 | CG | PRO | | 397 | −15.187 | 120.261 | −54.817 | 1.00 | 59.66 | |
| ATOM | 1823 | C | PRO | | 397 | −17.641 | 118.166 | −54.655 | 1.00 | 57.97 | |
| ATOM | 1824 | O | PRO | | 397 | −18.292 | 117.753 | −53.703 | 1.00 | 58.47 | |
| ATOM | 1825 | N | SER | | 398 | −18.188 | 118.677 | −55.755 | 1.00 | 58.12 | |
| ATOM | 1826 | CA | SER | | 398 | −19.627 | 118.805 | −55.955 | 1.00 | 57.49 | |
| ATOM | 1827 | CB | SER | | 398 | −19.910 | 120.019 | −56.841 | 1.00 | 59.12 | |
| ATOM | 1828 | OG | SER | | 398 | −21.222 | 119.962 | −57.381 | 1.00 | 60.35 | |
| ATOM | 1829 | C | SER | | 398 | −20.202 | 117.557 | −56.613 | 1.00 | 57.12 | |
| ATOM | 1830 | O | SER | | 398 | −21.421 | 117.424 | −56.756 | 1.00 | 57.38 | |
| ATOM | 1831 | N | ASP | | 399 | −19.304 | 116.666 | −57.031 | 1.00 | 55.79 | |
| ATOM | 1832 | CA | ASP | | 399 | −19.642 | 115.409 | −57.688 | 1.00 | 53.64 | |
| ATOM | 1833 | CB | ASP | | 399 | −20.239 | 114.408 | −56.681 | 1.00 | 53.34 | |
| ATOM | 1834 | CG | ASP | | 399 | −20.167 | 112.961 | −57.175 | 1.00 | 54.18 | |
| ATOM | 1835 | OD1 | ASP | | 399 | −21.224 | 112.359 | −57.451 | 1.00 | 53.28 | |
| ATOM | 1836 | OD2 | ASP | | 399 | −19.041 | 112.421 | −57.295 | 1.00 | 54.72 | |
| ATOM | 1837 | C | ASP | | 399 | −20.590 | 115.605 | −58.868 | 1.00 | 52.33 | |
| ATOM | 1838 | O | ASP | | 399 | −20.341 | 116.429 | −59.720 | 1.00 | 52.52 | |
| ATOM | 1839 | N | ALA | | 400 | −21.681 | 114.852 | −58.906 | 1.00 | 50.77 | |
| ATOM | 1840 | CA | ALA | | 400 | −22.631 | 114.931 | −60.001 | 1.00 | 50.76 | |
| ATOM | 1841 | CB | ALA | | 400 | −23.868 | 114.070 | −59.687 | 1.00 | 45.81 | |
| ATOM | 1842 | C | ALA | | 400 | −23.067 | 116.335 | −60.443 | 1.00 | 52.56 | |
| ATOM | 1843 | O | ALA | | 400 | −23.289 | 116.553 | −61.625 | 1.00 | 53.16 | |
| ATOM | 1844 | N | LYS | | 401 | −23.206 | 117.290 | −59.532 | 1.00 | 54.96 | |
| ATOM | 1845 | CA | LYS | | 401 | −23.634 | 118.619 | −59.974 | 1.00 | 57.33 | |
| ATOM | 1846 | CB | LYS | | 401 | −23.497 | 119.636 | −58.838 | 1.00 | 58.45 | |
| ATOM | 1847 | CG | LYS | | 401 | −24.688 | 119.577 | −57.889 | 1.00 | 60.29 | |
| ATOM | 1848 | CD | LYS | | 401 | −24.318 | 119.955 | −56.464 | 1.00 | 62.86 | |
| ATOM | 1849 | CE | LYS | | 401 | −25.286 | 119.355 | −55.445 | 1.00 | 60.93 | |
| ATOM | 1850 | NZ | LYS | | 401 | −24.950 | 119.774 | −54.058 | 1.00 | 61.71 | |
| ATOM | 1851 | C | LYS | | 401 | −22.851 | 119.057 | −61.211 | 1.00 | 57.18 | |
| ATOM | 1852 | O | LYS | | 401 | −23.438 | 119.391 | −62.233 | 1.00 | 58.02 | |
| ATOM | 1853 | N | GLU | | 402 | −21.531 | 119.018 | −61.125 | 1.00 | 56.57 | |
| ATOM | 1854 | CA | GLU | | 402 | −20.693 | 119.375 | −62.250 | 1.00 | 57.62 | |
| ATOM | 1855 | CB | GLU | | 402 | −19.246 | 118.959 | −61.968 | 1.00 | 59.68 | |
| ATOM | 1856 | CG | GLU | | 402 | −18.392 | 120.067 | −61.410 | 1.00 | 61.99 | |
| ATOM | 1857 | CD | GLU | | 402 | −17.300 | 119.560 | −60.502 | 1.00 | 65.15 | |
| ATOM | 1858 | OE1 | GLU | | 402 | −16.447 | 118.777 | −60.971 | 1.00 | 66.65 | |
| ATOM | 1859 | OE2 | GLU | | 402 | −17.297 | 119.951 | −59.312 | 1.00 | 69.23 | |
| ATOM | 1860 | C | GLU | | 402 | −21.155 | 118.733 | −63.556 | 1.00 | 57.40 | |
| ATOM | 1861 | O | GLU | | 402 | −21.109 | 119.363 | −64.603 | 1.00 | 59.82 | |
| ATOM | 1862 | N | VAL | | 403 | −21.594 | 117.482 | −63.499 | 1.00 | 56.49 | |
| ATOM | 1863 | CA | VAL | | 403 | −22.032 | 116.770 | −64.700 | 1.00 | 56.82 | |
| ATOM | 1864 | CB | VAL | | 403 | −21.892 | 115.217 | −64.530 | 1.00 | 54.96 | |
| ATOM | 1865 | CG1 | VAL | | 403 | −22.520 | 114.482 | −65.703 | 1.00 | 50.91 | |
| ATOM | 1866 | CG2 | VAL | | 403 | −20.420 | 114.858 | −64.398 | 1.00 | 52.81 | |
| ATOM | 1867 | C | VAL | | 403 | −23.455 | 117.115 | −65.063 | 1.00 | 57.58 | |
| ATOM | 1868 | O | VAL | | 403 | −23.810 | 117.172 | −66.232 | 1.00 | 59.33 | |
| ATOM | 1869 | N | MET | | 404 | −24.283 | 117.336 | −64.061 | 1.00 | 58.50 | |
| ATOM | 1870 | CA | MET | | 404 | −25.662 | 117.710 | −64.322 | 1.00 | 59.50 | |
| ATOM | 1871 | CB | MET | | 404 | −26.456 | 117.677 | −63.028 | 1.00 | 59.06 | |
| ATOM | 1872 | CG | MET | | 404 | −27.071 | 116.338 | −62.746 | 1.00 | 60.52 | |
| ATOM | 1873 | SD | MET | | 404 | −27.833 | 116.352 | −61.173 | 1.00 | 62.79 | |
| ATOM | 1874 | CE | MET | | 404 | −26.339 | 116.511 | −60.161 | 1.00 | 60.54 | |
| ATOM | 1875 | C | MET | | 404 | −25.696 | 119.117 | −64.921 | 1.00 | 60.09 | |
| ATOM | 1876 | O | MET | | 404 | −26.549 | 119.436 | −65.745 | 1.00 | 58.33 | |
| ATOM | 1877 | N | GLU | | 405 | −24.730 | 119.935 | −64.518 | 1.00 | 61.13 | |
| ATOM | 1878 | CA | GLU | | 405 | −24.634 | 121.308 | −64.977 | 1.00 | 63.25 | |
| ATOM | 1879 | CB | GLU | | 405 | −24.262 | 122.205 | −63.807 | 1.00 | 64.98 | |
| ATOM | 1880 | CG | GLU | | 405 | −25.212 | 122.114 | −62.663 | 1.00 | 68.67 | |
| ATOM | 1881 | CD | GLU | | 405 | −24.829 | 123.062 | −61.576 | 1.00 | 71.27 | |
| ATOM | 1882 | OE1 | GLU | | 405 | −23.642 | 123.033 | −61.169 | 1.00 | 72.33 | |
| ATOM | 1883 | OE2 | GLU | | 405 | −25.708 | 123.836 | −61.134 | 1.00 | 73.78 | |
| ATOM | 1884 | C | GLU | | 405 | −23.659 | 121.578 | −66.126 | 1.00 | 63.54 | |
| ATOM | 1885 | O | GLU | | 405 | −23.259 | 122.734 | −66.339 | 1.00 | 64.29 | |
| ATOM | 1886 | N | HIS | | 406 | −23.262 | 120.537 | −66.858 | 1.00 | 61.74 | |
| ATOM | 1887 | CA | HIS | | 406 | −22.364 | 120.746 | −67.988 | 1.00 | 58.19 | |
| ATOM | 1888 | CB | HIS | | 406 | −21.586 | 119.475 | −68.369 | 1.00 | 53.04 | |
| ATOM | 1889 | CG | HIS | | 406 | −20.810 | 119.609 | −69.645 | 1.00 | 47.77 | |
| ATOM | 1890 | CD2 | HIS | | 406 | −19.573 | 120.110 | −69.886 | 1.00 | 45.07 | |
| ATOM | 1891 | ND1 | HIS | | 406 | −21.345 | 119.294 | −70.879 | 1.00 | 44.27 | |
| ATOM | 1892 | CE1 | HIS | | 406 | −20.472 | 119.601 | −71.824 | 1.00 | 44.31 | |
| ATOM | 1893 | NE2 | HIS | | 406 | −19.390 | 120.099 | −71.250 | 1.00 | 43.15 | |
| ATOM | 1894 | C | HIS | | 406 | −23.187 | 121.185 | −69.181 | 1.00 | 58.97 | |
| ATOM | 1895 | O | HIS | | 406 | −24.339 | 120.764 | −69.355 | 1.00 | 57.85 | |
| ATOM | 1896 | N | ARG | | 407 | −22.564 | 122.028 | −69.998 | 1.00 | 59.59 | |
| ATOM | 1897 | CA | ARG | | 407 | −23.167 | 122.565 | −71.205 | 1.00 | 60.40 | |
| ATOM | 1898 | CB | ARG | | 407 | −22.063 | 123.126 | −72.112 | 1.00 | 62.98 | |
| ATOM | 1899 | CG | ARG | | 407 | −22.553 | 123.584 | −73.474 | 1.00 | 67.27 | |
| ATOM | 1900 | CD | ARG | | 407 | −21.392 | 124.001 | −74.366 | 1.00 | 72.05 | |
| ATOM | 1901 | NE | ARG | | 407 | −21.840 | 124.293 | −75.730 | 1.00 | 77.01 | |
| ATOM | 1902 | CZ | ARG | | 407 | −21.165 | 125.042 | −76.602 | 1.00 | 79.85 | |
| ATOM | 1903 | NH1 | ARG | | 407 | −19.997 | 125.589 | −76.257 | 1.00 | 80.74 | |
| ATOM | 1904 | NH2 | ARG | | 407 | −21.661 | 125.245 | −77.822 | 1.00 | 80.37 | |
| ATOM | 1905 | C | ARG | | 407 | −24.010 | 121.542 | −71.976 | 1.00 | 58.41 | |
| ATOM | 1906 | O | ARG | | 407 | −25.114 | 121.874 | −72.432 | 1.00 | 58.61 | |
| ATOM | 1907 | N | PHE | | 408 | −23.492 | 120.315 | −72.116 | 1.00 | 54.72 | |
| ATOM | 1908 | CA | PHE | | 408 | −24.175 | 119.246 | −72.852 | 1.00 | 51.67 | |
| ATOM | 1909 | CB | PHE | | 408 | −23.315 | 117.953 | −72.912 | 1.00 | 50.96 | |
| ATOM | 1910 | CG | PHE | | 408 | −24.011 | 116.792 | −73.614 | 1.00 | 45.30 | |
| ATOM | 1911 | CD1 | PHE | | 408 | −24.103 | 116.753 | −74.996 | 1.00 | 44.86 | |
| ATOM | 1912 | CD2 | PHE | | 408 | −24.700 | 115.834 | −72.880 | 1.00 | 41.97 | |
| ATOM | 1913 | CE1 | PHE | | 408 | −24.881 | 115.780 | −75.643 | 1.00 | 44.63 | |
| ATOM | 1914 | CE2 | PHE | | 408 | −25.483 | 114.860 | −73.509 | 1.00 | 40.86 | |
| ATOM | 1915 | CZ | PHE | | 408 | −25.574 | 114.834 | −74.896 | 1.00 | 42.82 | |
| ATOM | 1916 | C | PHE | | 408 | −25.531 | 118.877 | −72.290 | 1.00 | 52.59 | |
| ATOM | 1917 | O | PHE | | 408 | −26.377 | 118.361 | −73.012 | 1.00 | 50.75 | |
| ATOM | 1918 | N | PHE | | 409 | −25.736 | 119.142 | −71.002 | 1.00 | 53.94 | |
| ATOM | 1919 | CA | PHE | | 409 | −26.978 | 118.776 | −70.338 | 1.00 | 56.44 | |
| ATOM | 1920 | CB | PHE | | 409 | −26.654 | 118.021 | −69.038 | 1.00 | 56.94 | |
| ATOM | 1921 | CG | PHE | | 409 | −26.034 | 116.651 | −69.239 | 1.00 | 55.15 | |
| ATOM | 1922 | CD1 | PHE | | 409 | −26.776 | 115.598 | −69.778 | 1.00 | 53.68 | |
| ATOM | 1923 | CD2 | PHE | | 409 | −24.736 | 116.398 | −68.810 | 1.00 | 54.15 | |
| ATOM | 1924 | CE1 | PHE | | 409 | −26.237 | 114.318 | −69.873 | 1.00 | 51.49 | |
| ATOM | 1925 | CE2 | PHE | | 409 | −24.195 | 115.116 | −68.905 | 1.00 | 53.05 | |
| ATOM | 1926 | CZ | PHE | | 409 | −24.955 | 114.081 | −69.437 | 1.00 | 49.81 | |
| ATOM | 1927 | C | PHE | | 409 | −27.939 | 119.926 | −70.009 | 1.00 | 58.91 | |
| ATOM | 1928 | O | PHE | | 409 | −28.336 | 120.095 | −68.846 | 1.00 | 58.32 | |
| ATOM | 1929 | N | LEU | | 410 | −28.327 | 120.705 | −71.018 | 1.00 | 60.17 | |
| ATOM | 1930 | CA | LEU | | 410 | −29.252 | 121.819 | −70.797 | 1.00 | 61.03 | |
| ATOM | 1931 | CB | LEU | | 410 | −29.066 | 122.887 | −71.868 | 1.00 | 62.18 | |
| ATOM | 1932 | CG | LEU | | 410 | −30.276 | 123.781 | −72.119 | 1.00 | 62.82 | |
| ATOM | 1933 | CD1 | LEU | | 410 | −29.781 | 125.191 | −72.337 | 1.00 | 63.59 | |
| ATOM | 1934 | CD2 | LEU | | 410 | −31.095 | 123.263 | −73.314 | 1.00 | 61.19 | |
| ATOM | 1935 | C | LEU | | 410 | −30.710 | 121.381 | −70.788 | 1.00 | 60.51 | |
| ATOM | 1936 | O | LEU | | 410 | −31.413 | 121.574 | −69.799 | 1.00 | 61.04 | |
| ATOM | 1937 | N | SER | | 411 | −31.149 | 120.802 | −71.898 | 1.00 | 59.14 | |
| ATOM | 1938 | CA | SER | | 411 | −32.512 | 120.323 | −72.058 | 1.00 | 59.17 | |
| ATOM | 1939 | CB | SER | | 411 | −32.659 | 119.679 | −73.426 | 1.00 | 61.25 | |
| ATOM | 1940 | OG | SER | | 411 | −31.790 | 118.557 | −73.550 | 1.00 | 62.98 | |
| ATOM | 1941 | C | SER | | 411 | −32.864 | 119.277 | −71.020 | 1.00 | 59.35 | |
| ATOM | 1942 | O | SER | | 411 | −33.792 | 118.484 | −71.213 | 1.00 | 59.64 | |
| ATOM | 1943 | N | ILE | | 412 | −32.137 | 119.275 | −69.914 | 1.00 | 58.52 | |
| ATOM | 1944 | CA | ILE | | 412 | −32.347 | 118.256 | −68.909 | 1.00 | 57.09 | |
| ATOM | 1945 | CB | ILE | | 412 | −30.965 | 117.681 | −68.469 | 1.00 | 55.80 | |
| ATOM | 1946 | CG2 | ILE | | 412 | −31.129 | 116.665 | −67.341 | 1.00 | 54.27 | |
| ATOM | 1947 | CG1 | ILE | | 412 | −30.279 | 117.039 | −69.687 | 1.00 | 53.36 | |
| ATOM | 1948 | CD1 | ILE | | 412 | −31.207 | 116.117 | −70.531 | 1.00 | 47.19 | |
| ATOM | 1949 | C | ILE | | 412 | −33.180 | 118.611 | −67.697 | 1.00 | 56.60 | |
| ATOM | 1950 | O | ILE | | 412 | −32.866 | 119.527 | −66.946 | 1.00 | 56.94 | |
| ATOM | 1951 | N | ASN | | 413 | −34.256 | 117.859 | −67.516 | 1.00 | 56.83 | |
| ATOM | 1952 | CA | ASN | | 413 | −35.139 | 118.054 | −66.374 | 1.00 | 56.44 | |
| ATOM | 1953 | CB | ASN | | 413 | −36.609 | 118.029 | −66.807 | 1.00 | 56.52 | |
| ATOM | 1954 | CG | ASN | | 413 | −37.497 | 118.734 | −65.807 | 1.00 | 57.09 | |
| ATOM | 1955 | OD1 | ASN | | 413 | −38.330 | 118.116 | −65.126 | 1.00 | 54.58 | |
| ATOM | 1956 | ND2 | ASN | | 413 | −37.297 | 120.044 | −65.687 | 1.00 | 58.16 | |
| ATOM | 1957 | C | ASN | | 413 | −34.870 | 116.926 | −65.375 | 1.00 | 54.26 | |
| ATOM | 1958 | O | ASN | | 413 | −35.589 | 115.909 | −65.336 | 1.00 | 50.66 | |
| ATOM | 1959 | N | TRP | | 414 | −33.838 | 117.132 | −64.564 | 1.00 | 54.29 | |
| ATOM | 1960 | CA | TRP | | 414 | −33.406 | 116.132 | −63.596 | 1.00 | 56.88 | |
| ATOM | 1961 | CB | TRP | | 414 | −32.261 | 116.699 | −62.759 | 1.00 | 59.59 | |
| ATOM | 1962 | CG | TRP | | 414 | −31.001 | 116.991 | −63.578 | 1.00 | 65.04 | |
| ATOM | 1963 | CD2 | TRP | | 414 | −30.231 | 116.061 | −64.370 | 1.00 | 66.58 | |
| ATOM | 1964 | CE2 | TRP | | 414 | −29.129 | 116.778 | −64.904 | 1.00 | 67.20 | |
| ATOM | 1965 | CE3 | TRP | | 414 | −30.363 | 114.705 | −64.677 | 1.00 | 67.03 | |
| ATOM | 1966 | CD1 | TRP | | 414 | −30.348 | 118.190 | −63.671 | 1.00 | 67.27 | |
| ATOM | 1967 | NE1 | TRP | | 414 | −29.226 | 118.067 | −64.465 | 1.00 | 68.59 | |
| ATOM | 1968 | CZ2 | TRP | | 414 | −28.161 | 116.172 | −65.725 | 1.00 | 65.83 | |
| ATOM | 1969 | CZ3 | TRP | | 414 | −29.391 | 114.105 | −65.502 | 1.00 | 67.18 | |
| ATOM | 1970 | CH2 | TRP | | 414 | −28.309 | 114.844 | −66.011 | 1.00 | 65.33 | |
| ATOM | 1971 | C | TRP | | 414 | −34.481 | 115.516 | −62.699 | 1.00 | 56.82 | |
| ATOM | 1972 | O | TRP | | 414 | −34.258 | 114.480 | −62.116 | 1.00 | 58.02 | |
| ATOM | 1973 | N | GLN | | 415 | −35.653 | 116.136 | −62.605 | 1.00 | 57.42 | |
| ATOM | 1974 | CA | GLN | | 415 | −36.733 | 115.608 | −61.782 | 1.00 | 54.54 | |
| ATOM | 1975 | CB | GLN | | 415 | −37.658 | 116.724 | −61.304 | 1.00 | 54.51 | |
| ATOM | 1976 | CG | GLN | | 415 | −37.491 | 117.064 | −59.868 | 1.00 | 53.45 | |
| ATOM | 1977 | CD | GLN | | 415 | −38.620 | 117.895 | −59.319 | 1.00 | 52.95 | |
| ATOM | 1978 | OE1 | GLN | | 415 | −39.775 | 117.483 | −59.319 | 1.00 | 55.09 | |
| ATOM | 1979 | NE2 | GLN | | 415 | −38.286 | 119.064 | −58.821 | 1.00 | 52.40 | |
| ATOM | 1980 | C | GLN | | 415 | −37.532 | 114.650 | −62.619 | 1.00 | 55.66 | |
| ATOM | 1981 | O | GLN | | 415 | −38.101 | 113.698 | −62.108 | 1.00 | 54.58 | |
| ATOM | 1982 | N | ASP | | 416 | −37.585 | 114.917 | −63.916 | 1.00 | 56.96 | |
| ATOM | 1983 | CA | ASP | | 416 | −38.328 | 114.061 | −64.827 | 1.00 | 59.08 | |
| ATOM | 1984 | CB | ASP | | 416 | −38.469 | 114.714 | −66.189 | 1.00 | 63.17 | |
| ATOM | 1985 | CG | ASP | | 416 | −39.587 | 115.700 | −66.232 | 1.00 | 65.07 | |
| ATOM | 1986 | OD1 | ASP | | 416 | −40.718 | 115.322 | −65.832 | 1.00 | 66.66 | |
| ATOM | 1987 | OD2 | ASP | | 416 | −39.326 | 116.841 | −66.673 | 1.00 | 65.92 | |
| ATOM | 1988 | C | ASP | | 416 | −37.632 | 112.747 | −65.012 | 1.00 | 58.15 | |
| ATOM | 1989 | O | ASP | | 416 | −38.271 | 111.713 | −65.194 | 1.00 | 57.45 | |
| ATOM | 1990 | N | VAL | | 417 | −36.308 | 112.807 | −64.992 | 1.00 | 58.07 | |
| ATOM | 1991 | CA | VAL | | 417 | −35.494 | 111.611 | −65.141 | 1.00 | 57.45 | |
| ATOM | 1992 | CB | VAL | | 417 | −34.027 | 111.919 | −65.001 | 1.00 | 56.19 | |
| ATOM | 1993 | CG1 | VAL | | 417 | −33.252 | 110.664 | −65.241 | 1.00 | 57.02 | |
| ATOM | 1994 | CG2 | VAL | | 417 | −33.627 | 113.023 | −65.970 | 1.00 | 54.44 | |
| ATOM | 1995 | C | VAL | | 417 | −35.845 | 110.580 | −64.084 | 1.00 | 57.37 | |
| ATOM | 1996 | O | VAL | | 417 | −36.406 | 109.539 | −64.411 | 1.00 | 57.05 | |
| ATOM | 1997 | N | VAL | | 418 | −35.535 | 110.876 | −62.820 | 1.00 | 57.88 | |
| ATOM | 1998 | CA | VAL | | 418 | −35.821 | 109.929 | −61.739 | 1.00 | 60.11 | |
| ATOM | 1999 | CB | VAL | | 418 | −35.553 | 110.522 | −60.343 | 1.00 | 58.28 | |
| ATOM | 2000 | CG1 | VAL | | 418 | −34.146 | 110.989 | −60.244 | 1.00 | 58.15 | |
| ATOM | 2001 | CG2 | VAL | | 418 | −36.505 | 111.658 | −60.077 | 1.00 | 59.19 | |
| ATOM | 2002 | C | VAL | | 418 | −37.276 | 109.530 | −61.780 | 1.00 | 61.81 | |
| ATOM | 2003 | O | VAL | | 418 | −37.676 | 108.486 | −61.247 | 1.00 | 63.52 | |
| ATOM | 2004 | N | GLN | | 419 | −38.068 | 110.368 | −62.425 | 1.00 | 61.85 | |
| ATOM | 2005 | CA | GLN | | 419 | −39.484 | 110.117 | −62.501 | 1.00 | 65.20 | |
| ATOM | 2006 | CB | GLN | | 419 | −40.211 | 111.459 | −62.562 | 1.00 | 66.41 | |
| ATOM | 2007 | CG | GLN | | 419 | −40.141 | 112.262 | −61.265 | 1.00 | 67.19 | |
| ATOM | 2008 | CD | GLN | | 419 | −40.918 | 111.616 | −60.145 | 1.00 | 69.64 | |
| ATOM | 2009 | OE1 | GLN | | 419 | −42.108 | 111.329 | −60.284 | 1.00 | 72.38 | |
| ATOM | 2010 | NE2 | GLN | | 419 | −40.254 | 111.386 | −59.021 | 1.00 | 70.84 | |
| ATOM | 2011 | C | GLN | | 419 | −39.925 | 109.210 | −63.651 | 1.00 | 66.54 | |
| ATOM | 2012 | O | GLN | | 419 | −41.101 | 108.862 | −63.743 | 1.00 | 67.18 | |
| ATOM | 2013 | N | LYS | | 420 | −38.986 | 108.798 | −64.502 | 1.00 | 68.03 | |
| ATOM | 2014 | CA | LYS | | 420 | −39.314 | 107.959 | −65.651 | 1.00 | 67.91 | |
| ATOM | 2015 | CB | LYS | | 420 | −39.885 | 106.610 | −65.199 | 1.00 | 67.82 | |
| ATOM | 2016 | CG | LYS | | 420 | −38.854 | 105.707 | −64.557 | 1.00 | 67.76 | |
| ATOM | 2017 | CD | LYS | | 420 | −39.145 | 104.232 | −64.797 | 1.00 | 66.82 | |
| ATOM | 2018 | CE | LYS | | 420 | −37.855 | 103.439 | −64.661 | 1.00 | 67.85 | |
| ATOM | 2019 | NZ | LYS | | 420 | −38.036 | 101.982 | −64.890 | 1.00 | 66.83 | |
| ATOM | 2020 | C | LYS | | 420 | −40.347 | 108.738 | −66.454 | 1.00 | 68.30 | |
| ATOM | 2021 | O | LYS | | 420 | −41.243 | 108.172 | −67.099 | 1.00 | 67.93 | |
| ATOM | 2022 | N | LYS | | 421 | −40.200 | 110.055 | −66.382 | 1.00 | 68.73 | |
| ATOM | 2023 | CA | LYS | | 421 | −41.084 | 110.993 | −67.052 | 1.00 | 70.80 | |
| ATOM | 2024 | CB | LYS | | 421 | −41.093 | 112.333 | −66.302 | 1.00 | 72.18 | |
| ATOM | 2025 | CG | LYS | | 421 | −42.469 | 112.725 | −65.754 | 1.00 | 75.21 | |
| ATOM | 2026 | CD | LYS | | 421 | −43.105 | 111.599 | −64.918 | 1.00 | 77.43 | |
| ATOM | 2027 | CE | LYS | | 421 | −44.591 | 111.858 | −64.618 | 1.00 | 78.67 | |
| ATOM | 2028 | NZ | LYS | | 421 | −44.864 | 113.113 | −63.853 | 1.00 | 79.81 | |
| ATOM | 2029 | C | LYS | | 421 | −40.679 | 111.199 | −68.505 | 1.00 | 70.49 | |
| ATOM | 2030 | O | LYS | | 421 | −41.530 | 111.117 | −69.395 | 1.00 | 70.30 | |
| ATOM | 2031 | N | LEU | | 422 | −39.394 | 111.465 | −68.750 | 1.00 | 69.36 | |
| ATOM | 2032 | CA | LEU | | 422 | −38.923 | 111.652 | −70.122 | 1.00 | 67.65 | |
| ATOM | 2033 | CB | LEU | | 422 | −37.397 | 111.583 | −70.210 | 1.00 | 66.12 | |
| ATOM | 2034 | CG | LEU | | 422 | −36.538 | 112.611 | −69.467 | 1.00 | 66.13 | |
| ATOM | 2035 | CD1 | LEU | | 422 | −35.097 | 112.377 | −69.864 | 1.00 | 65.76 | |
| ATOM | 2036 | CD2 | LEU | | 422 | −36.942 | 114.039 | −69.801 | 1.00 | 65.12 | |
| ATOM | 2037 | C | LEU | | 422 | −39.505 | 110.525 | −70.959 | 1.00 | 66.67 | |
| ATOM | 2038 | O | LEU | | 422 | −39.916 | 109.488 | −70.428 | 1.00 | 65.65 | |
| ATOM | 2039 | N | LEU | | 423 | −39.579 | 110.733 | −72.263 | 1.00 | 66.27 | |
| ATOM | 2040 | CA | LEU | | 423 | −40.096 | 109.688 | −73.121 | 1.00 | 66.60 | |
| ATOM | 2041 | CB | LEU | | 423 | −40.965 | 110.266 | −74.254 | 1.00 | 65.11 | |
| ATOM | 2042 | CG | LEU | | 423 | −41.521 | 109.187 | −75.209 | 1.00 | 65.13 | |
| ATOM | 2043 | CD1 | LEU | | 423 | −42.684 | 108.433 | −74.548 | 1.00 | 62.44 | |
| ATOM | 2044 | CD2 | LEU | | 423 | −41.961 | 109.815 | −76.513 | 1.00 | 64.03 | |
| ATOM | 2045 | C | LEU | | 423 | −38.883 | 108.956 | −73.698 | 1.00 | 66.75 | |
| ATOM | 2046 | O | LEU | | 423 | −37.938 | 109.577 | −74.183 | 1.00 | 67.08 | |
| ATOM | 2047 | N | PRO | | 424 | −38.879 | 107.626 | −73.627 | 1.00 | 67.08 | |
| ATOM | 2048 | CD | PRO | | 424 | −39.933 | 106.682 | −73.206 | 1.00 | 67.32 | |
| ATOM | 2049 | CA | PRO | | 424 | −37.723 | 106.922 | −74.181 | 1.00 | 67.83 | |
| ATOM | 2050 | CB | PRO | | 424 | −38.013 | 105.464 | −73.824 | 1.00 | 67.11 | |
| ATOM | 2051 | CG | PRO | | 424 | −39.525 | 105.402 | −73.908 | 1.00 | 67.45 | |
| ATOM | 2052 | C | PRO | | 424 | −37.682 | 107.168 | −75.694 | 1.00 | 68.28 | |
| ATOM | 2053 | O | PRO | | 424 | −38.681 | 106.942 | −76.391 | 1.00 | 68.67 | |
| ATOM | 2054 | N | PRO | | 425 | −36.543 | 107.668 | −76.212 | 1.00 | 67.74 | |
| ATOM | 2055 | CD | PRO | | 425 | −35.442 | 108.255 | −75.431 | 1.00 | 67.36 | |
| ATOM | 2056 | CA | PRO | | 425 | −36.367 | 107.948 | −77.643 | 1.00 | 67.05 | |
| ATOM | 2057 | CB | PRO | | 425 | −34.996 | 108.641 | −77.706 | 1.00 | 66.39 | |
| ATOM | 2058 | CG | PRO | | 425 | −34.327 | 108.260 | −76.431 | 1.00 | 66.57 | |
| ATOM | 2059 | C | PRO | | 425 | −36.461 | 106.728 | −78.556 | 1.00 | 66.83 | |
| ATOM | 2060 | O | PRO | | 425 | −36.675 | 106.851 | −79.760 | 1.00 | 67.38 | |
| ATOM | 2061 | N | PHE | | 426 | −36.294 | 105.549 | −77.984 | 1.00 | 66.92 | |
| ATOM | 2062 | CA | PHE | | 426 | −36.392 | 104.322 | −78.752 | 1.00 | 67.44 | |
| ATOM | 2063 | CB | PHE | | 426 | −35.000 | 103.789 | −79.085 | 1.00 | 68.71 | |
| ATOM | 2064 | CG | PHE | | 426 | −34.976 | 102.330 | −79.402 | 1.00 | 68.61 | |
| ATOM | 2065 | CD1 | PHE | | 426 | −35.783 | 101.811 | −80.402 | 1.00 | 68.75 | |
| ATOM | 2066 | CD2 | PHE | | 426 | −34.213 | 101.462 | −78.641 | 1.00 | 68.76 | |
| ATOM | 2067 | CE1 | PHE | | 426 | −35.840 | 100.443 | −80.632 | 1.00 | 69.27 | |
| ATOM | 2068 | CE2 | PHE | | 426 | −34.263 | 100.096 | −78.862 | 1.00 | 69.47 | |
| ATOM | 2069 | CZ | PHE | | 426 | −35.081 | 99.583 | −79.860 | 1.00 | 69.98 | |
| ATOM | 2070 | C | PHE | | 426 | −37.143 | 103.355 | −77.864 | 1.00 | 68.25 | |
| ATOM | 2071 | O | PHE | | 426 | −36.813 | 103.201 | −76.692 | 1.00 | 69.44 | |
| ATOM | 2072 | N | LYS | | 427 | −38.158 | 102.706 | −78.403 | 1.00 | 68.40 | |
| ATOM | 2073 | CA | LYS | | 427 | −38.933 | 101.802 | −77.588 | 1.00 | 70.12 | |
| ATOM | 2074 | CB | LYS | | 427 | −40.401 | 102.215 | −77.609 | 1.00 | 71.43 | |
| ATOM | 2075 | CG | LYS | | 427 | −41.350 | 101.193 | −77.006 | 1.00 | 74.17 | |
| ATOM | 2076 | CD | LYS | | 427 | −42.120 | 101.795 | −75.838 | 1.00 | 76.63 | |
| ATOM | 2077 | CE | LYS | | 427 | −43.040 | 100.776 | −75.170 | 1.00 | 76.97 | |
| ATOM | 2078 | NZ | LYS | | 427 | −43.801 | 101.410 | −74.046 | 1.00 | 77.57 | |
| ATOM | 2079 | C | LYS | | 427 | −38.824 | 100.355 | −77.997 | 1.00 | 71.38 | |
| ATOM | 2080 | O | LYS | | 427 | −39.300 | 99.971 | −79.064 | 1.00 | 71.46 | |
| ATOM | 2081 | N | PRO | | 428 | −38.187 | 99.526 | −77.148 | 1.00 | 72.96 | |
| ATOM | 2082 | CD | PRO | | 428 | −37.468 | 99.936 | −75.928 | 1.00 | 72.02 | |
| ATOM | 2083 | CA | PRO | | 428 | −38.008 | 98.091 | −77.395 | 1.00 | 73.63 | |
| ATOM | 2084 | CB | PRO | | 428 | −37.523 | 97.583 | −76.053 | 1.00 | 73.04 | |
| ATOM | 2085 | CG | PRO | | 428 | −36.640 | 98.716 | −75.610 | 1.00 | 72.25 | |
| ATOM | 2086 | C | PRO | | 428 | −39.351 | 97.507 | −77.798 | 1.00 | 75.14 | |
| ATOM | 2087 | O | PRO | | 428 | −40.396 | 98.005 | −77.383 | 1.00 | 75.21 | |
| ATOM | 2088 | N | GLN | | 429 | −39.322 | 96.449 | −78.593 | 1.00 | 76.75 | |
| ATOM | 2089 | CA | GLN | | 429 | −40.551 | 95.865 | −79.100 | 1.00 | 79.71 | |
| ATOM | 2090 | CB | GLN | | 429 | −40.317 | 95.497 | −80.571 | 1.00 | 80.94 | |
| ATOM | 2091 | CG | GLN | | 429 | −39.747 | 96.692 | −81.355 | 1.00 | 82.23 | |
| ATOM | 2092 | CD | GLN | | 429 | −38.917 | 96.322 | −82.589 | 1.00 | 84.38 | |
| ATOM | 2093 | OE1 | GLN | | 429 | −38.118 | 95.374 | −82.572 | 1.00 | 84.60 | |
| ATOM | 2094 | NE2 | GLN | | 429 | −39.084 | 97.098 | −83.662 | 1.00 | 84.18 | |
| ATOM | 2095 | C | GLN | | 429 | −41.156 | 94.703 | −78.317 | 1.00 | 81.21 | |
| ATOM | 2096 | O | GLN | | 429 | −41.342 | 93.611 | −78.850 | 1.00 | 81.60 | |
| ATOM | 2097 | N | VAL | | 430 | −41.498 | 94.955 | −77.055 | 1.00 | 83.92 | |
| ATOM | 2098 | CA | VAL | | 430 | −42.097 | 93.922 | −76.206 | 1.00 | 87.13 | |
| ATOM | 2099 | CB | VAL | | 430 | −41.211 | 93.626 | −74.961 | 1.00 | 86.96 | |
| ATOM | 2100 | CG1 | VAL | | 430 | −39.913 | 92.962 | −75.395 | 1.00 | 87.00 | |
| ATOM | 2101 | CG2 | VAL | | 430 | −40.923 | 94.908 | −74.201 | 1.00 | 86.29 | |
| ATOM | 2102 | C | VAL | | 430 | −43.519 | 94.251 | −75.722 | 1.00 | 89.08 | |
| ATOM | 2103 | O | VAL | | 430 | −43.827 | 95.411 | −75.408 | 1.00 | 89.39 | |
| ATOM | 2104 | N | THR | | 431 | −44.372 | 93.221 | −75.671 | 1.00 | 90.54 | |
| ATOM | 2105 | CA | THR | | 431 | −45.769 | 93.353 | −75.223 | 1.00 | 91.77 | |
| ATOM | 2106 | CB | THR | | 431 | −46.566 | 92.037 | −75.446 | 1.00 | 91.58 | |
| ATOM | 2107 | OG1 | THR | | 431 | −46.459 | 91.626 | −76.814 | 1.00 | 90.98 | |
| ATOM | 2108 | CG2 | THR | | 431 | −48.038 | 92.237 | −75.094 | 1.00 | 92.07 | |
| ATOM | 2109 | C | THR | | 431 | −45.789 | 93.658 | −73.726 | 1.00 | 92.67 | |
| ATOM | 2110 | O | THR | | 431 | −46.132 | 94.764 | −73.295 | 1.00 | 91.94 | |
| ATOM | 2111 | N | SER | | 432 | −45.419 | 92.645 | −72.946 | 1.00 | 94.41 | |
| ATOM | 2112 | CA | SER | | 432 | −45.351 | 92.739 | −71.491 | 1.00 | 95.94 | |
| ATOM | 2113 | CB | SER | | 432 | −46.421 | 91.850 | −70.844 | 1.00 | 95.92 | |
| ATOM | 2114 | OG | SER | | 432 | −46.179 | 90.474 | −71.095 | 1.00 | 96.12 | |
| ATOM | 2115 | C | SER | | 432 | −43.965 | 92.270 | −71.055 | 1.00 | 96.83 | |
| ATOM | 2116 | O | SER | | 432 | −43.225 | 91.683 | −71.851 | 1.00 | 97.29 | |
| ATOM | 2117 | N | GLU | | 433 | −43.615 | 92.524 | −69.797 | 1.00 | 97.40 | |
| ATOM | 2118 | CA | GLU | | 433 | −42.314 | 92.119 | −69.278 | 1.00 | 98.03 | |
| ATOM | 2119 | CB | GLU | | 433 | −42.152 | 92.608 | −67.836 | 1.00 | 98.40 | |
| ATOM | 2120 | CG | GLU | | 433 | −43.433 | 92.581 | −67.033 | 1.00 | 100.35 | |
| ATOM | 2121 | CD | GLU | | 433 | −43.393 | 93.526 | −65.847 | 1.00 | 101.56 | |
| ATOM | 2122 | OE1 | GLU | | 433 | −42.595 | 93.289 | −64.910 | 1.00 | 102.03 | |
| ATOM | 2123 | OE2 | GLU | | 433 | −44.164 | 94.513 | −65.859 | 1.00 | 102.14 | |
| ATOM | 2124 | C | GLU | | 433 | −42.084 | 90.610 | −69.369 | 1.00 | 98.07 | |
| ATOM | 2125 | O | GLU | | 433 | −40.996 | 90.119 | −69.064 | 1.00 | 97.73 | |
| ATOM | 2126 | N | VAL | | 434 | −43.108 | 89.882 | −69.805 | 1.00 | 98.00 | |
| ATOM | 2127 | CA | VAL | | 434 | −43.014 | 88.436 | −69.958 | 1.00 | 98.10 | |
| ATOM | 2128 | CB | VAL | | 434 | −44.341 | 87.748 | −69.603 | 1.00 | 98.68 | |
| ATOM | 2129 | CG1 | VAL | | 434 | −44.198 | 86.239 | −69.712 | 1.00 | 98.59 | |
| ATOM | 2130 | CG2 | VAL | | 434 | −44.761 | 88.148 | −68.203 | 1.00 | 99.00 | |
| ATOM | 2131 | C | VAL | | 434 | −42.673 | 88.111 | −71.405 | 1.00 | 97.97 | |
| ATOM | 2132 | O | VAL | | 434 | −42.399 | 86.960 | −71.742 | 1.00 | 98.01 | |
| ATOM | 2133 | N | ASP | | 435 | −42.703 | 89.132 | −72.258 | 0.00 | 98.37 | |
| ATOM | 2134 | CA | ASP | | 435 | −42.385 | 88.963 | −73.671 | 1.00 | 98.70 | |
| ATOM | 2135 | CB | ASP | | 435 | −42.677 | 90.264 | −74.434 | 1.00 | 99.53 | |
| ATOM | 2136 | CG | ASP | | 435 | −42.591 | 90.090 | −75.940 | 1.00 | 100.16 | |
| ATOM | 2137 | OD1 | ASP | | 435 | −43.270 | 89.187 | −76.471 | 1.00 | 100.60 | |
| ATOM | 2138 | OD2 | ASP | | 435 | −41.849 | 90.858 | −76.594 | 1.00 | 100.18 | |
| ATOM | 2139 | C | ASP | | 435 | −40.901 | 88.600 | −73.749 | 1.00 | 98.21 | |
| ATOM | 2140 | O | ASP | | 435 | −40.027 | 89.463 | −73.678 | 1.00 | 96.94 | |
| ATOM | 2141 | N | THR | | 436 | −40.628 | 87.310 | −73.898 | 1.00 | 98.29 | |
| ATOM | 2142 | CA | THR | | 436 | −39.259 | 86.817 | −73.935 | 1.00 | 98.83 | |
| ATOM | 2143 | CB | THR | | 436 | −39.177 | 85.398 | −73.327 | 1.00 | 98.41 | |
| ATOM | 2144 | OG1 | THR | | 436 | −39.806 | 84.458 | −74.208 | 1.00 | 98.06 | |
| ATOM | 2145 | CG2 | THR | | 436 | −39.869 | 85.360 | −71.979 | 1.00 | 97.72 | |
| ATOM | 2146 | C | THR | | 436 | −38.584 | 86.764 | −75.299 | 1.00 | 99.29 | |
| ATOM | 2147 | O | THR | | 436 | −37.995 | 85.739 | −75.650 | 1.00 | 99.89 | |
| ATOM | 2148 | N | ARG | | 437 | −38.637 | 87.844 | −76.072 | 1.00 | 99.63 | |
| ATOM | 2149 | CA | ARG | | 437 | −37.990 | 87.788 | −77.376 | 1.00 | 100.28 | |
| ATOM | 2150 | CB | ARG | | 437 | −38.763 | 88.587 | −78.429 | 1.00 | 101.13 | |
| ATOM | 2151 | CG | ARG | | 437 | −38.677 | 90.098 | −78.355 | 1.00 | 101.69 | |
| ATOM | 2152 | CD | ARG | | 437 | −39.574 | 90.642 | −79.444 | 1.00 | 102.76 | |
| ATOM | 2153 | NE | ARG | | 437 | −40.849 | 89.921 | −79.422 | 1.00 | 104.17 | |
| ATOM | 2154 | CZ | ARG | | 437 | −41.742 | 89.912 | −80.409 | 1.00 | 104.28 | |
| ATOM | 2155 | NH1 | ARG | | 437 | −41.514 | 90.593 | −81.525 | 1.00 | 104.34 | |
| ATOM | 2156 | NH2 | ARG | | 437 | −42.861 | 89.207 | −80.283 | 1.00 | 103.71 | |
| ATOM | 2157 | C | ARG | | 437 | −36.546 | 88.239 | −77.343 | 1.00 | 100.10 | |
| ATOM | 2158 | O | ARG | | 437 | −35.704 | 87.652 | −78.019 | 1.00 | 100.44 | |
| ATOM | 2159 | N | TYR | | 438 | −36.246 | 89.265 | −76.557 | 1.00 | 99.91 | |
| ATOM | 2160 | CA | TYR | | 438 | −34.871 | 89.738 | −76.477 | 1.00 | 100.03 | |
| ATOM | 2161 | CB | TYR | | 438 | −34.802 | 91.122 | −75.831 | 1.00 | 99.89 | |
| ATOM | 2162 | CG | TYR | | 438 | −35.278 | 92.236 | −76.727 | 1.00 | 99.94 | |
| ATOM | 2163 | CD1 | TYR | | 438 | −36.610 | 92.645 | −76.719 | 1.00 | 99.48 | |
| ATOM | 2164 | CE1 | TYR | | 438 | −37.055 | 93.650 | −77.567 | 1.00 | 99.75 | |
| ATOM | 2165 | CD2 | TYR | | 438 | −34.398 | 92.865 | −77.611 | 1.00 | 100.01 | |
| ATOM | 2166 | CE2 | TYR | | 438 | −34.831 | 93.869 | −78.466 | 1.00 | 99.53 | |
| ATOM | 2167 | CZ | TYR | | 438 | −36.160 | 94.255 | −78.440 | 1.00 | 99.78 | |
| ATOM | 2168 | OH | TYR | | 438 | −36.599 | 95.234 | −79.300 | 1.00 | 101.08 | |
| ATOM | 2169 | C | TYR | | 438 | −33.987 | 88.776 | −75.695 | 1.00 | 100.06 | |
| ATOM | 2170 | O | TYR | | 438 | −32.919 | 89.164 | −75.221 | 1.00 | 100.26 | |
| ATOM | 2171 | N | PHE | | 439 | −34.426 | 87.524 | −75.569 | 1.00 | 99.97 | |
| ATOM | 2172 | CA | PHE | | 439 | −33.662 | 86.522 | −74.830 | 1.00 | 100.18 | |
| ATOM | 2173 | CB | PHE | | 439 | −34.596 | 85.725 | −73.910 | 1.00 | 99.09 | |
| ATOM | 2174 | CG | PHE | | 439 | −35.216 | 86.547 | −72.796 | 1.00 | 98.42 | |
| ATOM | 2175 | CD1 | PHE | | 439 | −36.189 | 85.994 | −71.967 | 1.00 | 98.25 | |
| ATOM | 2176 | CD2 | PHE | | 439 | −34.833 | 87.868 | −72.577 | 1.00 | 98.50 | |
| ATOM | 2177 | CE1 | PHE | | 439 | −36.773 | 86.739 | −70.943 | 1.00 | 97.85 | |
| ATOM | 2178 | CE2 | PHE | | 439 | −35.411 | 88.620 | −71.557 | 1.00 | 97.63 | |
| ATOM | 2179 | CZ | PHE | | 439 | −36.382 | 88.052 | −70.739 | 1.00 | 97.96 | |
| ATOM | 2180 | C | PHE | | 439 | −32.857 | 85.571 | −75.717 | 1.00 | 100.83 | |
| ATOM | 2181 | O | PHE | | 439 | −32.461 | 84.495 | −75.279 | 1.00 | 100.96 | |
| ATOM | 2182 | N | ASP | | 440 | −32.615 | 85.975 | −76.961 | 1.00 | 102.02 | |
| ATOM | 2183 | CA | ASP | | 440 | −31.832 | 85.177 | −77.902 | 1.00 | 103.27 | |
| ATOM | 2184 | CB | ASP | | 440 | −32.709 | 84.713 | −79.066 | 1.00 | 103.42 | |
| ATOM | 2185 | CG | ASP | | 440 | −33.772 | 83.719 | −78.633 | 1.00 | 103.54 | |
| ATOM | 2186 | OD1 | ASP | | 440 | −34.618 | 84.083 | −77.791 | 1.00 | 103.91 | |
| ATOM | 2187 | OD2 | ASP | | 440 | −33.761 | 82.574 | −79.133 | 1.00 | 103.74 | |
| ATOM | 2188 | C | ASP | | 440 | −30.650 | 86.010 | −78.415 | 1.00 | 104.64 | |
| ATOM | 2189 | O | ASP | | 440 | −29.738 | 86.315 | −77.644 | 1.00 | 105.47 | |
| ATOM | 2190 | N | ASP | | 441 | −30.654 | 86.381 | −79.698 | 1.00 | 105.56 | |
| ATOM | 2191 | CA | ASP | | 441 | −29.563 | 87.193 | −80.259 | 1.00 | 106.36 | |
| ATOM | 2192 | CB | ASP | | 441 | −28.606 | 86.330 | −81.085 | 1.00 | 107.59 | |
| ATOM | 2193 | CG | ASP | | 441 | −27.486 | 85.745 | −80.257 | 1.00 | 108.98 | |
| ATOM | 2194 | OD1 | ASP | | 441 | −27.749 | 84.815 | −79.459 | 1.00 | 109.39 | |
| ATOM | 2195 | OD2 | ASP | | 441 | −26.339 | 86.227 | −80.405 | 1.00 | 109.38 | |
| ATOM | 2196 | C | ASP | | 441 | −30.034 | 88.355 | −81.129 | 1.00 | 106.32 | |
| ATOM | 2197 | O | ASP | | 441 | −29.458 | 88.538 | −82.226 | 1.00 | 105.62 | |
| ATOM | 2198 | OXT | ASP | | 441 | −30.956 | 89.080 | −80.694 | 1.00 | 106.44 | |
| ATOM | 2199 | OH2 | TIP | S | 1 | −27.623 | 117.071 | −74.436 | 1.00 | 50.08 | S |
| ATOM | 2200 | OH2 | TIP | S | 2 | −6.363 | 114.551 | −69.692 | 1.00 | 52.44 | S |
| ATOM | 2201 | OH2 | TIP | S | 3 | −30.995 | 107.519 | −76.234 | 1.00 | 49.63 | S |
| ATOM | 2202 | OH2 | TIP | S | 4 | −14.629 | 98.513 | −69.834 | 1.00 | 49.61 | S |
| ATOM | 2203 | OH2 | TIP | S | 5 | −20.407 | 90.237 | −73.881 | 1.00 | 46.15 | S |
| ATOM | 2204 | OH2 | TIP | S | 6 | −42.857 | 114.573 | −63.472 | 1.00 | 98.81 | S |
| ATOM | 2205 | OH2 | TIP | S | 7 | −32.915 | 115.097 | −69.111 | 1.00 | 49.65 | S |
| ATOM | 2206 | OH2 | TIP | S | 8 | −24.764 | 110.454 | −73.650 | 1.00 | 61.01 | S |
| ATOM | 2207 | OH2 | TIP | S | 9 | −23.310 | 107.488 | −74.515 | 1.00 | 109.59 | S |
| ATOM | 2208 | OH2 | TIP | S | 10 | −10.718 | 83.937 | −50.781 | 1.00 | 57.03 | S |
| ATOM | 2209 | OH2 | TIP | S | 11 | −20.355 | 123.203 | −69.628 | 1.00 | 57.71 | S |
| ATOM | 2210 | OH2 | TIP | S | 12 | −29.683 | 79.225 | −62.379 | 1.00 | 60.08 | S |
| ATOM | 2211 | OH2 | TIP | S | 13 | −15.022 | 108.281 | −72.715 | 1.00 | 101.82 | S |
| ATOM | 2212 | OH2 | TIP | S | 14 | −30.449 | 120.422 | −66.954 | 1.00 | 51.43 | S |
| ATOM | 2213 | OH2 | TIP | S | 15 | −20.564 | 93.070 | −63.487 | 1.00 | 56.25 | S |
| ATOM | 2214 | OH2 | TIP | S | 16 | −15.157 | 102.558 | −64.601 | 1.00 | 29.10 | S |
| ATOM | 2215 | OH2 | TIP | S | 17 | −17.616 | 103.074 | −63.099 | 1.00 | 38.17 | S |
| ATOM | 2216 | OH2 | TIP | S | 18 | −2.634 | 106.999 | −59.689 | 1.00 | 45.82 | S |
| ATOM | 2217 | OH2 | TIP | S | 19 | −39.218 | 100.823 | −60.421 | 1.00 | 67.49 | S |
| ATOM | 2218 | OH2 | TIP | S | 20 | −23.554 | 97.041 | −63.102 | 1.00 | 56.11 | S |
| ATOM | 2219 | OH2 | TIP | S | 21 | −24.036 | 113.009 | −71.653 | 1.00 | 115.47 | S |
| ATOM | 2220 | OH2 | TIP | S | 22 | −17.643 | 98.286 | −74.324 | 1.00 | 46.61 | S |
| ATOM | 2221 | OH2 | TIP | S | 23 | −4.263 | 95.763 | −72.959 | 1.00 | 63.95 | S |
| ATOM | 2222 | OH2 | TIP | S | 24 | −25.175 | 95.708 | −73.068 | 1.00 | 52.58 | S |
| ATOM | 2223 | OH2 | TIP | S | 25 | −31.683 | 109.565 | −81.647 | 1.00 | 71.71 | S |
| ATOM | 2224 | OH2 | TIP | S | 26 | −31.483 | 113.973 | −74.194 | 1.00 | 38.69 | S |
| ATOM | 2225 | OH2 | TIP | S | 27 | −17.619 | 102.102 | −52.493 | 1.00 | 44.50 | S |
| ATOM | 2226 | OH2 | TIP | S | 28 | −25.520 | 91.421 | −58.993 | 1.00 | 84.60 | S |
| ATOM | 2227 | OH2 | TIP | S | 29 | −25.401 | 74.677 | −52.567 | 1.00 | 99.27 | S |
| ATOM | 2228 | OH2 | TIP | S | 30 | −19.973 | 111.319 | −52.542 | 1.00 | 49.17 | S |
| ATOM | 2229 | OH2 | TIP | S | 31 | −29.000 | 120.084 | −73.348 | 1.00 | 55.97 | S |
| ATOM | 2230 | OH2 | TIP | S | 32 | −17.714 | 100.955 | −71.187 | 1.00 | 62.36 | S |
| ATOM | 2231 | OH2 | TIP | S | 33 | −3.482 | 98.885 | −78.668 | 1.00 | 109.07 | S |
| ATOM | 2232 | OH2 | TIP | S | 34 | −50.390 | 95.737 | −54.746 | 1.00 | 55.63 | S |
| ATOM | 2233 | OH2 | TIP | S | 35 | −22.101 | 73.825 | −55.182 | 1.00 | 108.71 | S |
| ATOM | 2234 | OH2 | TIP | S | 36 | −18.664 | 123.812 | −72.193 | 1.00 | 49.19 | S |
| ATOM | 2235 | OH2 | TIP | S | 37 | −28.743 | 117.565 | −72.160 | 1.00 | 136.28 | S |
| ATOM | 2236 | OH2 | TIP | S | 38 | −22.769 | 112.806 | −73.840 | 1.00 | 62.32 | S |
| ATOM | 2237 | OH2 | TIP | S | 39 | −36.606 | 111.229 | −74.968 | 1.00 | 64.70 | S |
| ATOM | 2238 | OH2 | TIP | S | 40 | −31.320 | 98.646 | −45.930 | 1.00 | 103.40 | S |
| ATOM | 2239 | OH2 | TIP | S | 41 | −13.865 | 127.291 | −55.664 | 1.00 | 68.04 | S |
| ATOM | 2240 | OH2 | TIP | S | 42 | −25.892 | 121.805 | −67.521 | 1.00 | 63.93 | S |
| ATOM | 2241 | OH2 | TIP | S | 43 | −27.862 | 122.177 | −61.186 | 1.00 | 53.75 | S |
| ATOM | 2242 | OH2 | TIP | S | 44 | −21.137 | 80.560 | −57.614 | 1.00 | 97.15 | S |
| ATOM | 2243 | OH2 | TIP | S | 45 | −28.633 | 118.831 | −66.826 | 1.00 | 127.75 | S |
| ATOM | 2244 | OH2 | TIP | S | 46 | −15.616 | 96.600 | −54.071 | 1.00 | 71.96 | S |
| ATOM | 2245 | OH2 | TIP | S | 47 | −15.464 | 112.410 | −79.938 | 1.00 | 103.12 | S |
| ATOM | 2246 | OH2 | TIP | S | 48 | −27.144 | 117.541 | −79.370 | 1.00 | 38.56 | S |
| ATOM | 2247 | OH2 | TIP | S | 49 | −18.430 | 94.208 | −65.102 | 1.00 | 94.29 | S |
| ATOM | 2248 | OH2 | TIP | S | 50 | −41.024 | 98.946 | −60.461 | 1.00 | 56.05 | S |
| ATOM | 2249 | OH2 | TIP | S | 51 | −22.575 | 92.336 | −82.496 | 1.00 | 62.33 | S |
| ATOM | 2250 | OH2 | TIP | S | 52 | −27.415 | 89.929 | −85.543 | 1.00 | 54.98 | S |
| ATOM | 2251 | OH2 | TIP | S | 53 | −18.120 | 123.217 | −57.412 | 1.00 | 70.50 | S |
| ATOM | 2252 | OH2 | TIP | S | 54 | −18.647 | 93.038 | −70.008 | 1.00 | 62.14 | S |
| ATOM | 2253 | OH2 | TIP | S | 55 | −34.891 | 116.991 | −69.553 | 1.00 | 71.14 | S |
| ATOM | 2254 | OH2 | TIP | S | 56 | −24.452 | 109.908 | −62.339 | 1.00 | 82.11 | S |
| ATOM | 2255 | OH2 | TIP | S | 57 | −9.140 | 95.619 | −78.673 | 1.00 | 59.11 | S |
| ATOM | 2256 | OH2 | TIP | S | 58 | −22.630 | 77.355 | −64.914 | 1.00 | 54.06 | S |
| ATOM | 2257 | OH2 | TIP | S | 59 | −21.707 | 107.688 | −76.681 | 1.00 | 80.24 | S |
| ATOM | 2258 | OH2 | TIP | S | 60 | −25.218 | 92.131 | −45.479 | 1.00 | 65.39 | S |
| ATOM | 2259 | OH2 | TIP | S | 61 | −33.478 | 91.675 | −72.540 | 1.00 | 45.17 | S |
| ATOM | 2260 | OH2 | TIP | S | 62 | −6.638 | 100.525 | −89.582 | 1.00 | 57.51 | S |
| ATOM | 2261 | OH2 | TIP | S | 63 | −12.751 | 101.404 | −63.852 | 1.00 | 57.39 | S |
| ATOM | 2262 | OH2 | TIP | S | 64 | −16.620 | 90.320 | −50.785 | 1.00 | 65.96 | S |
| ATOM | 2263 | OH2 | TIP | S | 65 | −25.697 | 92.664 | −68.885 | 1.00 | 68.28 | S |
| ATOM | 2264 | OH2 | TIP | S | 66 | −15.025 | 118.312 | −89.771 | 1.00 | 58.18 | S |
| ATOM | 2265 | OH2 | TIP | S | 67 | −26.931 | 126.107 | −71.665 | 1.00 | 63.11 | S |
| ATOM | 2266 | OH2 | TIP | S | 68 | −34.795 | 103.669 | −75.122 | 1.00 | 72.18 | S |
| ATOM | 2267 | OH2 | TIP | S | 69 | −6.831 | 109.056 | −79.337 | 1.00 | 56.79 | S |
| ATOM | 2268 | OH2 | TIP | S | 70 | −40.479 | 91.734 | −58.440 | 1.00 | 70.62 | S |
| ATOM | 2269 | OH2 | TIP | S | 71 | −20.314 | 83.170 | −45.556 | 1.00 | 56.27 | S |
| ATOM | 2270 | OH2 | TIP | S | 72 | −26.070 | 77.761 | −66.849 | 1.00 | 72.74 | S |
| ATOM | 2271 | OH2 | TIP | S | 73 | −0.282 | 120.436 | −52.404 | 1.00 | 59.59 | S |
| ATOM | 2272 | OH2 | TIP | S | 74 | −32.286 | 112.812 | −61.349 | 1.00 | 43.83 | S |
| ATOM | 2273 | OH2 | TIP | S | 75 | −21.294 | 97.870 | −79.101 | 1.00 | 58.86 | S |
| ATOM | 2274 | OH2 | TIP | S | 76 | −27.673 | 75.363 | −70.379 | 1.00 | 76.66 | S |
| ATOM | 2275 | OH2 | TIP | S | 77 | −39.412 | 105.973 | −60.762 | 1.00 | 56.79 | S |
| ATOM | 2276 | OH2 | TIP | S | 78 | −20.303 | 89.918 | −51.002 | 1.00 | 64.33 | S |
| ATOM | 2277 | OH2 | TIP | S | 79 | −17.189 | 106.753 | −51.920 | 1.00 | 46.41 | S |
| ATOM | 2278 | OH2 | TIP | S | 80 | −47.843 | 80.379 | −64.135 | 1.00 | 60.37 | S |
| ATOM | 2279 | OH2 | TIP | S | 81 | −17.573 | 113.712 | −84.502 | 1.00 | 110.19 | S |
| ATOM | 2280 | OH2 | TIP | S | 82 | −37.065 | 102.989 | −56.757 | 1.00 | 54.22 | S |
| ATOM | 2281 | OH2 | TIP | S | 83 | −14.498 | 111.272 | −91.417 | 1.00 | 101.41 | S |
| ATOM | 2282 | OH2 | TIP | S | 84 | −30.372 | 106.961 | −82.277 | 1.00 | 70.02 | S |
| ATOM | 2283 | OH2 | TIP | S | 85 | −9.664 | 113.440 | −56.086 | 1.00 | 59.88 | S |
| ATOM | 2284 | OH2 | TIP | S | 86 | −24.964 | 98.917 | −55.078 | 1.00 | 55.96 | S |
| ATOM | 2285 | OH2 | TIP | S | 87 | −37.843 | 98.707 | −65.463 | 1.00 | 78.73 | S |
| ATOM | 2286 | OH2 | TIP | S | 88 | −31.274 | 103.379 | −51.826 | 1.00 | 127.66 | S |
| ATOM | 2287 | OH2 | TIP | S | 89 | −10.475 | 113.309 | −84.373 | 1.00 | 75.64 | S |
| ATOM | 2288 | OH2 | TIP | S | 90 | −27.341 | 123.774 | −70.092 | 1.00 | 71.65 | S |
| ATOM | 2289 | OH2 | TIP | S | 91 | −18.711 | 90.537 | −62.683 | 1.00 | 55.83 | S |
| ATOM | 2290 | OH2 | TIP | S | 92 | −33.403 | 106.130 | −77.985 | 1.00 | 78.72 | S |
| ATOM | 2291 | OH2 | TIP | S | 93 | −24.005 | 90.870 | −56.466 | 1.00 | 65.52 | S |
| ATOM | 2292 | OH2 | TIP | S | 94 | −27.617 | 104.616 | −54.961 | 1.00 | 111.64 | S |
| ATOM | 2293 | OH2 | TIP | S | 95 | −13.742 | 93.007 | −62.576 | 1.00 | 55.12 | S |
| ATOM | 2294 | OH2 | TIP | S | 96 | −26.852 | 118.188 | −57.257 | 1.00 | 50.05 | S |
| ATOM | 2295 | OH2 | TIP | S | 97 | −22.007 | 123.757 | −63.602 | 1.00 | 81.09 | S |
| ATOM | 2296 | OH2 | TIP | S | 98 | −23.230 | 88.728 | −66.431 | 1.00 | 110.10 | S |
| ATOM | 2297 | OH2 | TIP | S | 99 | −13.360 | 112.796 | −87.736 | 1.00 | 51.81 | S |
| ATOM | 2298 | OH2 | TIP | S | 100 | −16.221 | 123.986 | −53.826 | 1.00 | 64.19 | S |
| ATOM | 2299 | OH2 | TIP | S | 101 | −21.410 | 129.947 | −61.869 | 1.00 | 51.98 | S |
| ATOM | 2300 | OH2 | TIP | S | 102 | −10.815 | 115.537 | −81.525 | 1.00 | 79.49 | S |
| ATOM | 2301 | OH2 | TIP | S | 103 | −10.813 | 93.308 | −60.006 | 1.00 | 73.70 | S |
| ATOM | 2302 | OH2 | TIP | S | 104 | −40.267 | 90.357 | −71.550 | 1.00 | 102.57 | S |
| ATOM | 2303 | OH2 | TIP | S | 105 | −33.508 | 104.329 | −71.475 | 1.00 | 130.44 | S |
| ATOM | 2304 | OH2 | TIP | S | 106 | −18.744 | 92.097 | −73.138 | 1.00 | 57.33 | S |
| ATOM | 2305 | OH2 | TIP | S | 107 | −36.651 | 100.017 | −59.561 | 1.00 | 80.34 | S |
| ATOM | 2306 | OH2 | TIP | S | 108 | −35.655 | 83.715 | −66.690 | 1.00 | 92.34 | S |
| ATOM | 2307 | OH2 | TIP | S | 109 | −15.004 | 123.171 | −72.873 | 1.00 | 64.24 | S |
| ATOM | 2308 | OH2 | TIP | S | 110 | −19.809 | 86.420 | −75.382 | 1.00 | 60.00 | S |
| ATOM | 2309 | OH2 | TIP | S | 111 | −22.061 | 106.390 | −65.510 | 1.00 | 141.40 | S |
| ATOM | 2310 | OH2 | TIP | S | 112 | −30.065 | 119.754 | −61.428 | 1.00 | 54.41 | S |
| ATOM | 2311 | OH2 | TIP | S | 113 | −21.117 | 101.845 | −76.666 | 1.00 | 133.00 | S |
| ATOM | 2312 | OH2 | TIP | S | 114 | −36.169 | 94.852 | −53.914 | 1.00 | 52.74 | S |
| ATOM | 2313 | OH2 | TIP | S | 115 | −20.513 | 90.392 | −76.531 | 1.00 | 62.03 | S |
| ATOM | 2314 | OH2 | TIP | S | 116 | −7.874 | 116.642 | −87.594 | 1.00 | 65.17 | S |
| ATOM | 2315 | OH2 | TIP | S | 117 | −12.611 | 91.681 | −79.605 | 1.00 | 54.76 | S |
| ATOM | 2316 | OH2 | TIP | S | 118 | −39.602 | 93.998 | −67.457 | 1.00 | 74.47 | S |
| ATOM | 2317 | OH2 | TIP | S | 119 | −31.966 | 115.257 | −81.634 | 1.00 | 96.68 | S |
| ATOM | 2318 | OH2 | TIP | S | 120 | −20.353 | 112.035 | −73.437 | 1.00 | 110.82 | S |
| ATOM | 2319 | OH2 | TIP | S | 121 | −8.471 | 110.279 | −71.660 | 1.00 | 75.05 | S |
| ATOM | 2320 | OH2 | TIP | S | 122 | −19.609 | 72.745 | −59.575 | 1.00 | 57.01 | S |
| ATOM | 2321 | OH2 | TIP | S | 123 | −3.225 | 126.719 | −50.300 | 1.00 | 46.98 | S |
| ATOM | 2322 | OH2 | TIP | S | 124 | −13.531 | 110.225 | −57.101 | 1.00 | 47.35 | S |
| ATOM | 2323 | OH2 | TIP | S | 125 | −15.950 | 117.562 | −82.262 | 1.00 | 82.90 | S |
| ATOM | 2324 | OH2 | TIP | S | 126 | −9.300 | 127.283 | −56.135 | 1.00 | 64.58 | S |
| ATOM | 2325 | OH2 | TIP | S | 127 | −40.326 | 100.805 | −86.362 | 1.00 | 67.40 | S |
| ATOM | 2326 | OH2 | TIP | S | 128 | −26.743 | 109.839 | −47.135 | 1.00 | 56.64 | S |
| ATOM | 2327 | OH2 | TIP | S | 129 | −21.152 | 78.015 | −44.545 | 1.00 | 63.33 | S |
| ATOM | 2328 | OH2 | TIP | S | 130 | −15.502 | 111.092 | −65.948 | 1.00 | 93.70 | S |
| ATOM | 2329 | OH2 | TIP | S | 131 | −61.010 | 80.720 | −82.560 | 1.00 | 59.97 | S |
| ATOM | 2330 | OH2 | TIP | S | 132 | −5.754 | 114.545 | −88.180 | 1.00 | 46.35 | S |
| ATOM | 2331 | OH2 | TIP | S | 133 | −17.343 | 102.484 | −73.188 | 1.00 | 80.48 | S |
| ATOM | 2332 | OH2 | TIP | S | 134 | −22.668 | 100.024 | −66.101 | 1.00 | 160.50 | S |
| ATOM | 2333 | OH2 | TIP | S | 135 | −13.492 | 97.626 | −62.513 | 1.00 | 74.96 | S |
| ATOM | 2334 | OH2 | TIP | S | 136 | −34.975 | 118.383 | −60.826 | 1.00 | 54.24 | S |
| ATOM | 2335 | OH2 | TIP | S | 137 | −39.901 | 98.826 | −74.968 | 1.00 | 107.12 | S |
| ATOM | 2336 | OH2 | TIP | S | 138 | −13.081 | 90.301 | −74.716 | 1.00 | 80.68 | S |
| ATOM | 2337 | OH2 | TIP | S | 139 | −6.679 | 129.585 | −62.277 | 1.00 | 58.26 | S |
| ATOM | 2338 | OH2 | TIP | S | 140 | −24.229 | 112.256 | −55.630 | 1.00 | 42.51 | S |
| ATOM | 2339 | OH2 | TIP | S | 141 | −29.294 | 92.995 | −46.924 | 1.00 | 62.81 | S |
| ATOM | 2340 | OH2 | TIP | S | 142 | −33.181 | 120.270 | −61.770 | 1.00 | 58.91 | S |
| ATOM | 2341 | OH2 | TIP | S | 143 | −15.987 | 93.183 | −52.324 | 1.00 | 56.26 | S |
| ATOM | 2342 | OH2 | TIP | S | 144 | −33.793 | 120.291 | −64.846 | 1.00 | 128.61 | S |
| ATOM | 2343 | OH2 | TIP | S | 145 | −7.502 | 112.874 | −86.551 | 1.00 | 89.97 | S |
| ATOM | 2344 | OH2 | TIP | S | 146 | −28.737 | 113.816 | −70.544 | 1.00 | 90.60 | S |
| ATOM | 2345 | OH2 | TIP | S | 147 | −25.751 | 91.368 | −78.478 | 1.00 | 56.02 | S |
| ATOM | 2346 | OH2 | TIP | S | 148 | −5.538 | 108.115 | −57.303 | 1.00 | 61.91 | S |
| ATOM | 2347 | OH2 | TIP | S | 149 | −6.750 | 117.327 | −84.625 | 1.00 | 55.56 | S |
| ATOM | 2348 | OH2 | TIP | S | 150 | −6.064 | 113.190 | −80.641 | 1.00 | 84.73 | S |
| ATOM | 2349 | OH2 | TIP | S | 151 | −14.898 | 122.897 | −70.515 | 1.00 | 72.28 | S |
| ATOM | 2350 | OH2 | TIP | S | 152 | −17.385 | 119.055 | −80.821 | 1.00 | 117.81 | S |
| ATOM | 2351 | OH2 | TIP | S | 153 | −17.722 | 92.098 | −76.941 | 1.00 | 77.30 | S |
| ATOM | 2352 | OH2 | TIP | S | 154 | −41.186 | 101.886 | −63.398 | 1.00 | 70.46 | S |
| ATOM | 2353 | OH2 | TIP | S | 155 | −36.523 | 109.555 | −67.648 | 1.00 | 63.22 | S |
| ATOM | 2354 | OH2 | TIP | S | 156 | −46.348 | 79.889 | −42.332 | 1.00 | 70.93 | S |
| ATOM | 2355 | OH2 | TIP | S | 157 | −31.159 | 112.205 | −64.750 | 1.00 | 96.21 | S |
| ATOM | 2356 | OH2 | TIP | S | 158 | −16.496 | 98.729 | −51.917 | 1.00 | 68.11 | S |
| ATOM | 2357 | OH2 | TIP | S | 159 | −27.445 | 92.326 | −55.432 | 1.00 | 105.80 | S |
| ATOM | 2358 | OH2 | TIP | S | 160 | −12.883 | 122.829 | −51.350 | 1.00 | 52.37 | S |
| ATOM | 2359 | OH2 | TIP | S | 161 | −37.558 | 90.883 | −73.852 | 1.00 | 56.64 | S |
| ATOM | 2360 | OH2 | TIP | S | 162 | −32.439 | 111.015 | −77.093 | 1.00 | 95.14 | S |
| ATOM | 2361 | OH2 | TIP | S | 163 | −29.848 | 91.790 | −64.074 | 1.00 | 53.48 | S |
| ATOM | 2362 | OH2 | TIP | S | 164 | −47.202 | 94.576 | −65.895 | 1.00 | 62.74 | S |
| ATOM | 2363 | OH2 | TIP | S | 165 | −10.553 | 86.337 | −79.886 | 1.00 | 62.46 | S |
| ATOM | 2364 | OH2 | TIP | S | 166 | −28.158 | 117.033 | −76.907 | 1.00 | 50.72 | S |
| ATOM | 2365 | OH2 | TIP | S | 167 | −20.768 | 90.813 | −71.552 | 1.00 | 63.30 | S |
| ATOM | 2366 | OH2 | TIP | S | 168 | −42.018 | 80.510 | −57.537 | 1.00 | 72.58 | S |
| ATOM | 2367 | OH2 | TIP | S | 169 | −30.391 | 84.848 | −82.737 | 1.00 | 69.68 | S |
| ATOM | 2368 | OH2 | TIP | S | 170 | −4.077 | 114.307 | −69.426 | 1.00 | 63.76 | S |
| ATOM | 2369 | OH2 | TIP | S | 171 | −16.148 | 98.312 | −71.887 | 1.00 | 84.16 | S |
| ATOM | 2370 | OH2 | TIP | S | 172 | −19.294 | 101.248 | −73.895 | 1.00 | 115.91 | S |
| ATOM | 2371 | OH2 | TIP | S | 173 | −29.677 | 116.091 | −74.250 | 1.00 | 112.47 | S |
| ATOM | 2372 | OH2 | TIP | S | 174 | −16.280 | 101.239 | −66.115 | 1.00 | 51.98 | S |
| ATOM | 2373 | OH2 | TIP | S | 175 | −31.445 | 90.794 | −73.675 | 1.00 | 76.22 | S |
| ATOM | 2374 | OH2 | TIP | S | 176 | −37.325 | 101.693 | −52.478 | 1.00 | 115.74 | S |
| ATOM | 2375 | OH2 | TIP | S | 177 | −33.007 | 116.295 | −72.875 | 1.00 | 132.16 | S |
| ATOM | 2376 | OH2 | TIP | S | 178 | −33.602 | 108.930 | −56.008 | 1.00 | 78.57 | S |
| ATOM | 2377 | OH2 | TIP | S | 179 | −27.755 | 103.470 | −66.606 | 1.00 | 111.83 | S |
| ATOM | 2378 | OH2 | TIP | S | 180 | −23.711 | 110.440 | −82.309 | 1.00 | 52.25 | S |
| ATOM | 2379 | OH2 | TIP | S | 181 | −19.445 | 77.703 | −62.696 | 1.00 | 94.18 | S |
| ATOM | 2380 | OH2 | TIP | S | 182 | −40.075 | 101.931 | −67.249 | 1.00 | 53.34 | S |
| ATOM | 2381 | OH2 | TIP | S | 183 | −9.169 | 88.933 | −70.344 | 1.00 | 48.03 | S |
| ATOM | 2382 | OH2 | TIP | S | 184 | −7.205 | 111.603 | −56.621 | 1.00 | 53.47 | S |
| ATOM | 2383 | OH2 | TIP | S | 185 | −26.172 | 121.826 | −70.446 | 1.00 | 101.08 | S |
| ATOM | 2384 | OH2 | TIP | S | 186 | −17.802 | 111.367 | −89.651 | 1.00 | 66.17 | S |
| ATOM | 2385 | OH2 | TIP | S | 187 | −4.045 | 107.148 | −89.001 | 1.00 | 58.76 | S |
| ATOM | 2386 | OH2 | TIP | S | 188 | −28.598 | 115.945 | −80.483 | 1.00 | 91.65 | S |
| ATOM | 2387 | OH2 | TIP | S | 189 | −19.368 | 110.747 | −54.934 | 1.00 | 128.20 | S |
| ATOM | 2388 | OH2 | TIP | S | 190 | −20.905 | 75.083 | −60.333 | 1.00 | 91.67 | S |
| ATOM | 2389 | OH2 | TIP | S | 191 | −25.133 | 114.645 | −61.905 | 1.00 | 118.77 | S |
| ATOM | 2390 | OH2 | TIP | S | 192 | −10.847 | 108.163 | −58.161 | 1.00 | 55.70 | S |
| ATOM | 2391 | OH2 | TIP | S | 193 | −31.040 | 90.727 | −62.161 | 1.00 | 127.17 | S |
| ATOM | 2392 | OH2 | TIP | S | 194 | −24.361 | 88.949 | −61.408 | 1.00 | 96.03 | S |
| ATOM | 2393 | OH2 | TIP | S | 195 | −25.845 | 118.902 | −77.997 | 1.00 | 101.37 | S |
| ATOM | 2394 | OH2 | TIP | S | 196 | −45.257 | 100.264 | −64.437 | 1.00 | 67.73 | S |
| ATOM | 2395 | OH2 | TIP | S | 197 | −19.534 | 122.816 | −60.513 | 1.00 | 57.10 | S |
| ATOM | 2396 | OH2 | TIP | S | 198 | −17.729 | 89.087 | −73.164 | 1.00 | 62.32 | S |
| ATOM | 2397 | OH2 | TIP | S | 199 | −25.598 | 115.479 | −56.345 | 1.00 | 58.00 | S |
| ATOM | 2398 | OH2 | TIP | S | 200 | −11.966 | 110.289 | −86.531 | 1.00 | 58.38 | S |
| ATOM | 2399 | OH2 | TIP | S | 201 | −11.862 | 111.179 | −90.005 | 1.00 | 62.26 | S |
| ATOM | 2400 | OH2 | TIP | S | 202 | −23.146 | 103.334 | −74.388 | 1.00 | 86.36 | S |
| ATOM | 2401 | OH2 | TIP | S | 203 | −25.348 | 100.591 | −88.904 | 1.00 | 83.58 | S |
| ATOM | 2402 | OH2 | TIP | S | 204 | −33.579 | 99.455 | −57.461 | 1.00 | 123.22 | S |
| ATOM | 2403 | OH2 | TIP | S | 205 | −19.262 | 110.559 | −81.294 | 1.00 | 94.92 | S |
| ATOM | 2404 | OH2 | TIP | S | 206 | −3.814 | 105.699 | −53.606 | 1.00 | 59.43 | S |
| ATOM | 2405 | OH2 | TIP | S | 207 | −42.986 | 86.709 | −66.627 | 1.00 | 101.31 | S |
| ATOM | 2406 | OH2 | TIP | S | 208 | −1.519 | 111.809 | −84.036 | 1.00 | 50.96 | S |
| ATOM | 2407 | OH2 | TIP | S | 209 | −17.903 | 106.223 | −57.233 | 1.00 | 112.85 | S |
| ATOM | 2408 | OH2 | TIP | S | 210 | −39.670 | 95.363 | −88.124 | 1.00 | 59.37 | S |
| ATOM | 2409 | OH2 | TIP | S | 211 | −17.795 | 75.194 | −56.614 | 1.00 | 104.92 | S |
| ATOM | 2410 | OH2 | TIP | S | 212 | −20.005 | 112.200 | −78.821 | 1.00 | 110.72 | S |
| ATOM | 2411 | OH2 | TIP | S | 213 | −0.589 | 117.171 | −54.358 | 1.00 | 56.45 | S |
| ATOM | 2412 | OH2 | TIP | S | 214 | −30.338 | 92.585 | −76.714 | 1.00 | 110.56 | S |
| ATOM | 2413 | OH2 | TIP | S | 215 | −0.557 | 94.041 | −72.839 | 1.00 | 72.72 | S |
| ATOM | 2414 | OH2 | TIP | S | 216 | −44.272 | 80.569 | −64.137 | 1.00 | 88.29 | S |
| ATOM | 2415 | OH2 | TIP | S | 217 | −8.330 | 108.845 | −85.172 | 1.00 | 61.74 | S |
| ATOM | 2416 | OH2 | TIP | S | 218 | −38.922 | 101.691 | −58.270 | 1.00 | 100.23 | S |
| ATOM | 2417 | OH2 | TIP | S | 219 | −14.469 | 88.506 | −51.433 | 1.00 | 69.37 | S |
| ATOM | 2418 | OH2 | TIP | S | 220 | −15.575 | 95.883 | −51.472 | 1.00 | 92.96 | S |
| ATOM | 2419 | OH2 | TIP | S | 221 | −23.554 | 92.371 | −47.328 | 1.00 | 103.71 | S |
| ATOM | 2420 | OH2 | TIP | S | 222 | −3.867 | 118.046 | −85.348 | 1.00 | 77.94 | S |
| ATOM | 2421 | OH2 | TIP | S | 223 | −35.589 | 120.474 | −63.099 | 1.00 | 87.19 | S |
| ATOM | 2422 | OH2 | TIP | S | 224 | −44.239 | 110.368 | −58.934 | 1.00 | 77.76 | S |
| ATOM | 2423 | OH2 | TIP | S | 225 | −32.794 | 111.024 | −68.824 | 1.00 | 108.60 | S |
| ATOM | 2424 | OH2 | TIP | S | 226 | −22.720 | 80.213 | −59.846 | 1.00 | 130.32 | S |
| ATOM | 2425 | OH2 | TIP | S | 227 | −42.005 | 107.878 | −70.190 | 1.00 | 96.92 | S |
| ATOM | 2426 | OH2 | TIP | S | 228 | −15.709 | 78.715 | −43.542 | 1.00 | 58.73 | S |
| ATOM | 2427 | OH2 | TIP | S | 229 | −39.901 | 97.291 | −52.193 | 1.00 | 60.44 | S |
| ATOM | 2428 | OH2 | TIP | S | 230 | −33.181 | 83.277 | −46.966 | 1.00 | 74.04 | S |
| ATOM | 2429 | OH2 | TIP | S | 231 | −36.075 | 105.384 | −71.283 | 1.00 | 111.20 | S |
| ATOM | 2430 | OH2 | TIP | S | 232 | −23.807 | 124.303 | −80.685 | 1.00 | 57.30 | S |
| ATOM | 2431 | OH2 | TIP | S | 233 | −32.691 | 113.570 | −83.528 | 1.00 | 93.84 | S |
| ATOM | 2432 | OH2 | TIP | S | 234 | −35.384 | 101.526 | −65.535 | 1.00 | 74.54 | S |
| ATOM | 2433 | OH2 | TIP | S | 235 | −52.003 | 97.430 | −68.647 | 1.00 | 73.21 | S |
| ATOM | 2434 | OH2 | TIP | S | 236 | −53.636 | 92.100 | −47.728 | 1.00 | 83.96 | S |
| ATOM | 2435 | OH2 | TIP | S | 237 | −32.998 | 79.952 | −48.805 | 1.00 | 77.92 | S |
| ATOM | 2436 | OH2 | TIP | S | 238 | −19.609 | 88.765 | −71.863 | 1.00 | 63.44 | S |
| ATOM | 2437 | OH2 | TIP | S | 239 | −37.978 | 101.579 | −62.221 | 1.00 | 114.24 | S |
| ATOM | 2438 | OH2 | TIP | S | 240 | −40.568 | 92.296 | −86.674 | 1.00 | 65.76 | S |
| ATOM | 2439 | OH2 | TIP | S | 241 | −23.296 | 93.673 | −73.044 | 1.00 | 87.42 | S |
| ATOM | 2440 | OH2 | TIP | S | 242 | −18.241 | 102.306 | −50.261 | 1.00 | 99.29 | S |
| ATOM | 2441 | OH2 | TIP | S | 243 | −45.686 | 89.248 | −74.060 | 1.00 | 80.50 | S |
| ATOM | 2442 | OH2 | TIP | S | 244 | −48.990 | 77.329 | −65.285 | 1.00 | 62.79 | S |
| ATOM | 2443 | OH2 | TIP | S | 245 | −39.670 | 103.275 | −60.596 | 1.00 | 78.31 | S |
| ATOM | 2444 | OH2 | TIP | S | 246 | −49.281 | 84.180 | −38.445 | 1.00 | 62.88 | S |
| ATOM | 2445 | OH2 | TIP | S | 247 | −35.923 | 76.665 | −73.082 | 1.00 | 90.47 | S |
| ATOM | 2446 | OH2 | TIP | S | 248 | −22.650 | 98.270 | −55.329 | 1.00 | 66.55 | S |
| ATOM | 2447 | OH2 | TIP | S | 249 | −1.136 | 105.962 | −80.208 | 1.00 | 95.63 | S |
| ATOM | 2448 | OH2 | TIP | S | 250 | −30.948 | 81.763 | −46.481 | 1.00 | 64.39 | S |
| ATOM | 2449 | OH2 | TIP | S | 251 | −31.424 | 106.525 | −70.271 | 1.00 | 146.73 | S |
| ATOM | 2450 | OH2 | TIP | S | 252 | −47.586 | 98.613 | −55.205 | 1.00 | 71.91 | S |
| ATOM | 2451 | OH2 | TIP | S | 253 | −2.582 | 111.252 | −61.347 | 1.00 | 64.80 | S |
| ATOM | 2452 | OH2 | TIP | S | 254 | −32.001 | 99.414 | −81.548 | 1.00 | 75.73 | S |
| ATOM | 2453 | OH2 | TIP | S | 255 | −14.287 | 99.143 | −64.847 | 1.00 | 86.21 | S |
| ATOM | 2454 | OH2 | TIP | S | 256 | −54.384 | 78.808 | −65.841 | 1.00 | 53.97 | S |
| ATOM | 2455 | OH2 | TIP | S | 257 | −26.112 | 120.574 | −60.165 | 1.00 | 104.99 | S |
| ATOM | 2456 | OH2 | TIP | S | 258 | −45.064 | 97.484 | −65.973 | 1.00 | 70.27 | S |
| ATOM | 2457 | OH2 | TIP | S | 259 | −27.816 | 89.385 | −78.676 | 1.00 | 86.86 | S |
| ATOM | 2458 | OH2 | TIP | S | 260 | −46.644 | 107.061 | −62.630 | 1.00 | 70.71 | S |
| ATOM | 2459 | OH2 | TIP | S | 261 | −18.353 | 98.010 | −49.930 | 1.00 | 105.72 | S |
| ATOM | 2460 | OH2 | TIP | S | 262 | −8.406 | 116.226 | −81.494 | 1.00 | 104.02 | S |
| ATOM | 2461 | OH2 | TIP | S | 263 | −33.763 | 81.288 | −44.581 | 1.00 | 67.25 | S |
| ATOM | 2462 | OH2 | TIP | S | 264 | −14.153 | 87.119 | −63.758 | 1.00 | 70.68 | S |
| ATOM | 2463 | OH2 | TIP | S | 265 | −16.810 | 120.032 | −69.896 | 1.00 | 88.49 | S |
| ATOM | 2464 | OH2 | TIP | S | 266 | 0.697 | 78.048 | −53.011 | 1.00 | 79.10 | S |
| ATOM | 2465 | OH2 | TIP | S | 267 | −13.291 | 117.712 | −84.014 | 1.00 | 86.90 | S |
| ATOM | 2466 | OH2 | TIP | S | 268 | −41.914 | 95.291 | −85.766 | 1.00 | 66.46 | S |
| END | | | | | | | | | | | |

1. A crystal of PKBβ having a tetragonal space group P4₁2₁2, and unit cell dimensions of a=149.33±0.5 Å, b=149.33±0.5 Å, c=39.77±0.5 Å; a=148.40±0.5 Å, b=148.40±0.5 Å, c=38.55±0.5 Å; a=149.70±0.5 Å, b=149.70±0.5 Å, c=39.19±0.5 Å; or a=149.52±0.5 Å, b=149.52±0.5 Å, c=39.06±0.5 Å.

2. A crystal according to claim 1, having unit cell dimensions of a=149.33±0.2 Å, b=149.33±0.2 Å, c=39.77±0.2 Å; a=148.40±0.2 Å, b=148.40±0.2 Å, c=38.55±0.2 Å; a=149.70±0.2 Å, b=149.70±0.2 Å, c=39.19±0.2 Å; or a=149.52±0.2 Å, b=149.52±0.2 Å, c=39.06±0.2 Å.

3. A crystal according to claim 1 or claim 2, having unit cell dimensions of a=149.33 Å, b=149.33 Å, c=39.77 Å; a=148.40 Å, b=148.40 Å, c=38.55 Å; a=149.70 Å, b=149.70 Å, c=39.19 Å; or a=149.52 Å, b=149.52 Å, c=39.06 Å.

4. A crystal of PKBβ having the three dimensional atomic coordinates of any one of Tables 2 to 5.

5. A method for crystallizing a PKB derivative which comprises producing PKB by recombinant production in a host cell, recovering a PKB derivative from the host and growing crystals therefrom, wherein the PKB derivative is a stable protease-resistant form of PKB.

6. A method according to claim 5, wherein the PKB derivative lacks all or substantially all of the PH domain.

7. A method according to claim 6, wherein the PKB derivative has an N-terminus corresponding to Lys-146 of human PKBβ.

8. A method according to any one of claims 5 to 7, wherein the host cell is an insect cell.

9. A method according to any one of claims 5 to 8, further comprising the step of phosphorylating the PKB derivative in vitro.

10. A method according to claim 9, wherein the PKB derivative is phosphorylated at a residue corresponding to Thr-309 of human PKBβ.

11. A method according to claim 10, wherein said phosphorylation is performed with PDK1.

12. A method according to any one of claims 5 to 11, wherein the crystal is grown by the under oil batch method.

13. A method of determining the structure of a PKB derivative comprising the step of X-ray diffraction analysis of a crystal as produced by the method of any one of claims 5 to 12.

14. A PKB polypeptide having an N-terminus corresponding to Lys-146 of human PKBβ.

15. A PKB polypeptide according to claim 14, comprising a catalytic domain corresponding to residues 146 to 440 of human PKBβ.

16. A nucleic acid encoding a polypeptide according to claim 14 or claim 15.

17. A vector comprising a nucleic acid according to claim 16.

18. A host cell comprising a nucleic acid according to claim 16 or a vector according to claim 17.

19. A method of preparing a polypeptide according to claim 14 or claim 15, comprising the step of expressing said polypeptide from a nucleic acid according to claim 16.

20. A method of analysing a PKBβ-ligand complex comprising the step of employing (i) X-ray crystallographic diffraction data from the PKBβ-ligand complex and (ii) a three-dimensional structure of PKBβ to generate a difference Fourier electron density map of the complex, the three-dimensional structure being defined by atomic coordinate data according to any one of Tables 2 to 5.

21. A method of determining a three dimensional structure for a target kinase comprising the steps of:
(a) aligning a representation of an amino acid sequence of a target kinase of unknown structure with the amino acid sequence of PKBβ to match homologous regions of the amino acid sequences;
(b) modelling the structure of the matched homologous regions of the target kinase on the structure of the corresponding regions of PKBβ as defined by any one of Tables 2 to 5; and
(c) determining a conformation for the target kinase which substantially preserves the structure of said matched homologous regions.

22. A method for determining a three-dimensional structure for a target kinase, comprising the steps of; providing the co-ordinates of Tables 2 to 5, and positioning the co-ordinates in the crystal unit cell of said target kinase so as to provide a structure for said target kinase.

23. A method according to claim 21 or claim 22, wherein the target kinase is an AGC kinase, or a co-complex, derivative or mutant thereof.

24. A method according to claim 23, wherein the AGC kinase is PKBα or PKBγ, or a co-complex, derivative or mutant thereof.

25. A method for determining three-dimensional atomic coordinate data for a target conformation of a PKB isoform, comprising the steps of:
(a) employing three-dimensional atomic coordinate data of any one of Tables 2 to 5;
(b) employing three-dimensional atomic coordinate data of a template kinase structure, and:
(c) determining three-dimensional atomic coordinate data for said target conformation.

26. A method according to claim 25 wherein the template kinase structure is a structure of an AGC kinase.

27. A method according to claim 26 wherein the template kinase structure is a structure of murine PKA.

28. A computer system or computer-readable media containing either (a) atomic coordinate data according to Tables 2 to 5, said data defining the three-dimensional structure of PKB, or at least selected coordinates thereof; (b) structure factor data for PKB, said structure factor data being derivable from the atomic coordinate data of any one of Tables 2 to 5; (c) a Fourier transform of atomic coordinate data according to Tables 2 to 5, or at least selected coordinates thereof; (d) atomic coordinate data of a target kinase generated by homology modelling of the target based on the data of any one of Tables 2 to 5; (e) atomic coordinate data of a target kinase generated by interpreting X-ray crystallographic data or NMR data by reference to the data of any one of Tables 2 to 5; or (f) structure factor data derivable from the atomic coordinate data of (c), (d) or (e).

29. A method for modelling the interaction between PKB and an agent compound which modulates PKB activity, comprising the steps of:
(a) employing three-dimensional atomic coordinate data according to any one of Tables 2 to 5 to characterise at least one PKBβ binding site;
(b) providing the structure of said agent compound; and
(c) fitting said agent compound to the binding site.

30. A method according to claim 29, wherein the agent compound is a peptide.

31. A method according to claim 30, wherein the peptide comprises the sequence FXXF, YXXF, YXXY, FXXFX(Y/F), YXXFX(Y/F), YXXYX(Y/F), FXXFX′, FXXFX′ (F/Y), FXX′FX′, or FXX′FX′ (F/Y); YXXFX′, YXXFX′ (F/Y), YXX′FX′, or YXX′FX′ (F/Y); FXXYX′, FXXYX′ (F/Y), FXX′YX′, or FXX′YX′ (F/Y); YXXYX′, YXXYX′ (F/Y), YXX′YX′, or YXX′YX′ (F/Y), where X′ represents an amino acid residue which carries a negative charge at physiological pH..

32. A method according to claim 30 or 31, wherein the peptide comprises the sequence FPQFpSY (where pS is phosphoserine), FPQFDY, FRDFDY, GLLELDQRTHFPQFpSYSASIRE, GLLELDQRTHFPQFDYSASIRE or REPRILSEEEQEMFRDFDYIADWC.

33. A method for identifying an agent compound which modulates PKB activity, comprising the steps of:
(a) employing three-dimensional atomic coordinate data according to any one of Tables 2 to 5 to characterise at least one PKBβ binding site;
(b) providing the structure of a candidate agent compound;
(c) fitting the candidate agent compound to the binding site; and
(d) selecting the candidate agent compound.

34. A method according to claim 33 wherein:
a plurality of binding sites are characterised and a plurality of agent compounds are fitted to said sites; and
said agent compounds are linked to form a potential modulator compound.

35. A method according to claim 33 or 34 wherein step (b) comprises selecting said candidate agent compound by computationally screening a database of compounds for interaction with said binding site.

36. A method according to any one of claims 33 to 35 which comprises the further steps of:
(e) obtaining or synthesising the candidate agent compound; and
(f) contacting the candidate agent compound with PKB to determine the ability of the candidate agent compound to interact with PKB.

37. A method according to any one of claims 33 to 35 which comprises the further steps of:
(e) obtaining or synthesising the candidate agent compound;
(f) forming a complex of PKB and the candidate agent compound; and
(g) analysing said complex by X-ray crystallography or NMR spectroscopy to determine the ability of the candidate agent compound to interact with PKB.

38. A method according to any one of claims 33 to 37, wherein the binding site has previously been determined to bind a known agent compound by the method of any one of claims 29 to 32.

39. A method according to claim 38, wherein said known agent compound is a peptide comprising an activation motif, said activation motif comprising a hydrophobic motif.

40. A method according to claim 39, wherein said activation motif comprises the sequence FXXF, YXXF, YXXY, FXXFX(Y/F), YXXFX(Y/F), or YXXYX(Y/F).

41. A method according to claim 39 or claim 40, wherein said activation motif further comprises an amino acid residue which carries a negative charge at physiological pH.

42. A method according to any one of claims 39 to 41 wherein the activation motif comprises the sequence FXXFX′, FXXFX′ (F/Y), FXX′FX′, or FXX′FX′ (F/Y); YXXFX′, YXXFX′ (F/Y), YXX′FX′, or YXX′FX′ (F/Y); FXXYX′, FXXYX′ (F/Y), FXX′YX′, or FXX′YX′ (F/Y); YXXYX′, YXXYX′ (F/Y), YXX′YX′, or YXX′YX′ (F/Y); where X′ represents an amino acid residue which carries a negative charge at physiological pH.

43. A method according to any one of claims 39 to 42, wherein the activation motif comprises the sequence FPQFpSY, FPQFDY or FRDFDY, where pS is phosphoserine.

44. A method according to claim 43, wherein the peptide comprises the sequence GLLELDQRTHFPQFpSYSASIRE, GLLELDQRTHFPQFDYSASIRE and REPRILSEEEQEMFRDFDYIADWC, where pS is phosphoserine.

45. A compound which is identified as a modulator of PKB activity by the method of any one of claims 33 to 44.

46. A method of inducing a catalytic domain of an AGC kinase to adopt an active conformation, wherein the AGC kinase in its native form is regulated by phosphorylation of a regulatory phosphorylation site residue in a C-terminal regulatory segment distinct from said catalytic domain, said method comprising the steps of:
(a) providing a polypeptide comprising said catalytic domain, and
(b) forming a non-covalent complex between said polypeptide and an activating agent, wherein contact between said activating agent and said catalytic domain induces said catalytic domain to adopt an active conformation.

47. A method according to claim 46 wherein the catalytic domain comprises residues corresponding to residues 146 to 440 of human PKBβ.

48. A method according to claim 46 or claim 47, wherein said polypeptide is provided in a stable protease resistant form.

49. A method according to any one of claims 46 to 48, wherein said AGC kinase is PKB.

50. A method according to any one of claims 46 to 49, further comprising the step of phosphorylating said catalytic domain at a position corresponding to residue 309 of human PKBβ.

51. A method according to claim 50, wherein said phosphorylation is performed with PDK1.

52. A method according to any one of claims 46 to 51, wherein said activating agent is a peptide comprising an activation motif, said activation motif comprising a hydrophobic motif.

53. A method according to claim 52, wherein said activation motif comprises the sequence FXXF, YXXF, YXXY, FXXFX(Y/F), YXXFX(Y/F), or YXXYX(Y/F).

54. A method according to claim 52 or claim 53, wherein said activation motif further comprises an amino acid residue which carries a negative charge at physiological pH.

55. A method according to any one of claims 52 to 54 wherein the activation motif comprises the sequence FXXFX′, FXXFX′ (F/Y), FXX′FX′, or FXX′FX′ (F/Y); YXXFX′, YXXFX′ (F/Y), YXX′FX′, or YXX′FX′ (F/Y); FXXYX′, FXXYX′ (F/Y), FXX′YX′, or FXX′YX′ (F/Y); YXXYX′, YXXYX′ (F/Y), YXX′YX′, or YXX′YX′ (F/Y); where X′ represents an amino acid residue which carries a negative charge at physiological pH.

56. A method according to claim 55 wherein the activation motif comprises the sequence FPQFpSY, FPQFDY or FRDFDY, where pS is phosphoserine.

57. A method according to claim 56, wherein the peptide comprises the sequence GLLELDQRTHFPQFpSYSASIRE, GLLELDQRTHFPQFDYSASIRE and REPRILSEEEQEMFRDFDYIADWC, where pS is phosphoserine.

58. A non-covalent complex between a catalytic domain of an AGC kinase, wherein the AGC kinase in its native form is regulated by phosphorylation of a regulatory phosphorylation site residue in a C-terminal regulatory segment distinct from said catalytic domain, and an activating agent as described in any one of claims 52 to 57.

59. A method for determining the structure of an active conformation of a catalytic domain of an AGC kinase, wherein the AGC kinase in its native form is regulated by phosphorylation of a regulatory phosphorylation site residue in a C-terminal regulatory segment distinct from said catalytic domain, said method comprising the steps of inducing said catalytic domain to adopt an active conformation by the method of any one of claims 46 to 57, and obtaining a data set for said conformation, from which a structure may be calculated.

60. A method according to claim 59, wherein said AGC kinase is provided in a stable protease resistant form.

61. A method according to claim 59 or claim 60 wherein said AGC kinase is PKB lacking substantially all of the PH domain.

62. A method according to claim 61, wherein said PKB has an N-terminus corresponding to Lys-146 of human PKBβ.

63. A method according to any one of claims 59 to 62, further comprising the step of crystallising said catalytic domain in said active conformation.

64. A method according to claim 63, further comprising the step of X-ray crystallographic analysis of said crystal.

65. A method according to any one of claims 59 to 62, wherein said data set is acquired by NMR.

66. A method for assessing the ability of a candidate compound to modulate the catalytic activity of an AGC kinase, which in its native form is regulated by phosphorylation of a hydrophobic motif residue in a C-terminal regulatory segment, comprising the steps of
(a) providing a polypeptide comprising a catalytic domain of said kinase,
(b) forming a non-covalent complex between said polypeptide and an activating agent, wherein contact between said activating agent and said catalytic domain induces said catalytic domain to adopt an active conformation, and
(c) contacting said non-covalent complex with said candidate agent.

67. A method according to claim 66, further comprising the step of measuring the effect of the candidate agent on the AGC kinase activity.

68. A method according to claim 66 or claim 67, further comprising the step of phosphorylating said catalytic domain at a position corresponding to residue 309 of human PKBβ.

69. A method according to claim 68, wherein said phosphorylation is performed with PDK1.

70. A method of inducing a catalytic domain of an AGC kinase to adopt an active conformation, wherein the AGC kinase in its native form is regulated by phosphorylation of a regulatory phosphorylation site residue in a C-terminal regulatory segment distinct from said catalytic domain, said method comprising the steps of:
(a) providing a polypeptide comprising said catalytic domain, and
(b) covalently joining said polypeptide to an activating agent, wherein contact between said activating agent and said catalytic domain induces said catalytic domain to adopt an active conformation.

71. A method according to claim 70, wherein said activating agent is a peptide.

72. A method according to claim 71, wherein said peptide is a phosphopeptide.

73. A method according to claim 72, wherein said phosphopeptide comprises an activation motif sequence derived from the same AGC kinase as the catalytic domain.

74. A method according to claim 73, wherein said AGC kinase is PKB, and said phosphopeptide comprises the sequence FPGFpSY, where pS is phosphoserine.

75. A method according to claim 74, wherein the phosphopeptide comprises the sequence GLLELDQRTHFPQFpSYSASIRE.

76. A method for determining a structure for an active conformation of a catalytic domain of an AGC kinase, wherein the AGC kinase in its native form is regulated by phosphorylation of a regulatory phosphorylation site residue in a C-terminal regulatory segment distinct from said catalytic domain, said method comprising the steps of:
(a) providing a mutant AGC kinase protein comprising a catalytic domain and a C-terminal regulatory segment distinct from said catalytic domain, the protein further comprising a mutation which enhances the interaction between said regulatory segment and said catalytic domain relative to the wild type enzyme, such that an active conformation is induced in said catalytic domain, and
(b) obtaining a data set for said mutant protein from which a structure can be calculated.

77. A method according to claim 76, wherein a regulatory phosphorylation site is substituted with a residue which carries an electrostatic charge at physiological pH.

78. A method according to claim 76 or 77, wherein the a plurality of contiguous residues of the C-terminal regulatory segment are substituted by the corresponding residues from a second AGC kinase.

79. A method according to claim 78, wherein the second AGC kinase is PRK2.

80. A method according to any one of claims 76 to 79, wherein the sequence FRDFDY is introduced into the C-terminal regulatory segment.

81. A method according to claim 80, wherein the sequence REPRILSEEEQEMFRDFDYIADWC is introduced into the C-terminal regulatory segment.

82. A method according to any one of claims 76 to 81, wherein a mutation is made in the catalytic domain.

83. A method according to claim 82, wherein the mutation comprises the substitution of at least one of the residues corresponding to V194 and V198 of human PKBβ.

84. A method according to claim 83, wherein the mutation comprises at least one of V194I and V198L.

85. A method according to any one of claims 76 to 84, wherein said mutant AGC kinase is provided in a stable protease resistant form.

86. A method according to claim 85 wherein said mutant AGC kinase is PKB lacking substantially all of the PH domain.

87. A method according to claim 86, wherein said PKB has an N-terminus corresponding to Lys-146 of human PKBβ.

88. A method according to any one of claims 76 to 87, further comprising the step of crystallising said catalytic domain in said active conformation.

89. A method according to claim 88, further comprising the step of X-ray crystallographic analysis of said crystal.

90. A method according to any one of claims 76 to 87, wherein said data set is acquired by NMR.

91. A mutant AGC kinase protein, wherein the AGC kinase in its native form is regulated by phosphorylation of a regulatory phosphorylation site residue in a C-terminal regulatory segment, said mutant AGC kinase protein comprising a catalytic domain and a C-terminal regulatory segment distinct from said catalytic domain, and having an N-terminus corresponding to residue 139, 140, 141, 142, 143, 144, 145, 146, 147, 148, 149 or 150 of human PKBβ, or their corresponding residues in other isoforms, the mutant AGC kinase protein comprising a mutation which enhances the interaction between said regulatory segment and said catalytic domain relative to the wild type enzyme, such that an active conformation is induced in said catalytic domain.

92. A mutant AGC kinase according to claim 91, having an N-terminus corresponding to residue 146 of human PKBβ.

93. A mutant AGC kinase according to claim 91 or claim 92, wherein the mutation introduces a residue which carries an electrostatic charge at physiological pH into the C-terminal regulatory segment.

94. A mutant AGC kinase according to claim 93, wherein a regulatory phosphorylation site is substituted with a residue which carries an electrostatic charge at physiological pH.

95. A mutant AGC kinase according to any one of claims 91 to 94, wherein the a plurality of contiguous residues of the C-terminal regulatory segment are substituted by the corresponding residues from a second AGC kinase.

96. A mutant AGC kinase according to claim 95, wherein the second AGC kinase is PRK2.

97. A mutant AGC kinase according to claim 96, wherein the sequence FRDFDY is introduced into the C-terminal regulatory segment.

98. A mutant AGC kinase according to claim 97, wherein the sequence REPRILSEEEQEMFRDFDYIADWC is introduced into the C-terminal regulatory segment.

99. A mutant AGC kinase according to any one of claims 91 to 98, wherein a mutation is made in the catalytic domain.

100. A mutant AGC kinase according to claim 99, wherein the mutation comprises the substitution of at least one of the residues corresponding to V194 and V198 of human PKBβ.

101. A mutant AGC kinase according to claim 100, wherein the mutation comprises at least one of V194I and V198L.
